(19) United States
(12) Patent Application Publication
(10) Pub. No.: US 2025/0144140 A1
(43) Pub. Date: May 8, 2025

(54) METHODS FOR ALLOGENEIC HEMATOPOIETIC STEM CELL TRANSPLANTATION

(71) Applicant: Orca Biosystems, Inc., Menlo Park, CA (US)

(72) Inventors: Scott Killian, Menlo Park, CA (US); Scott McClellan, Menlo Park, CA (US); Amy Putnam, Menlo Park, CA (US); Nathaniel Fernhoff, Menlo Park, CA (US)

(21) Appl. No.: 18/916,371

(22) Filed: Oct. 15, 2024

Related U.S. Application Data
(63) Continuation of application No. PCT/US2023/018731, filed on Apr. 14, 2023.
(60) Provisional application No. 63/445,979, filed on Feb. 15, 2023.
(60) Provisional application No. 63/331,709, filed on Apr. 15, 2022.

(51) Int. Cl. A61K 35/17; A61K 31/436; A61K 35/28; A61K 40/11; A61K 40/42; A61P 35/02; C07K 14/705; C07K 14/715; C07K 14/725; C07K 14/73

(52) U.S. Cl. CPC A61K 35/17; A61K 31/436; A61K 35/28; A61K 40/11; A61K 40/421; A61K 40/4214; A61P 35/02; C07K 14/7051; C07K 14/70514; C07K 14/70589; C07K 14/715; A61K 2239/38; A61K 2239/48

(57) ABSTRACT

Various embodiments of the present disclosure provide therapeutic compositions and associated methods for improved hematopoietic stem cell transplantations, including methods to enhance protection from graft versus host disease while maintaining effective immune responses such as graft versus tumor immune responses.

### CROSS-REFERENCE TO RELATED APPLICATIONS

This application is a continuation of International Application No. PCT/US23/18731 filed Apr. 14, 2023, which claims the benefit of priority of U.S. Provisional Application Nos. 63/331,709 filed Apr. 15, 2022, and 63/445,979 filed Feb. 15, 2023, each of which is hereby incorporated by reference in their entirety for all purposes.

### BACKGROUND

Patients with hematologic malignancies such as leukemia and lymphoma beyond first remission or with refractory relapse are rarely cured with standard chemotherapy. Myeloablative allogeneic hematopoietic cell transplantation (alloHCT) is associated with improved survival in these patients. Myeloablative alloHCT is a procedure in which the patient undergoes chemotherapy or radiation to ablate or destroy tissue in the bone causing the malignancy. They then receive hematopoietic cells, including hematopoietic stem and progenitor cells (HSPC) from a donor's blood. However, alloHCT has a major drawback in that it often results in graft versus host disease (GVHD). GVHD is a condition in which the transplanted donor peripheral blood stem cells view the patient's body as foreign, and the donated cells attack the patient's tissue (e.g., skin, GI tissue, liver tissue, and lung tissue) resulting in a number of complications, many of which can be serious and result in morbidity and mortality. Thus, there is a need for improved methods and therapies for hematopoietic cell transplantation which have a reduced incidence and severity of GVHD including, reduced morbidity and mortality.

### BRIEF SUMMARY

Certain aspects of the present disclosure relate to a multi-component pharmaceutical treatment to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent.

In some embodiments that may be combined with any of the preceding embodiments, the GVHD prophylactic agent comprises tacrolimus and/or its analogues and derivatives which according to various embodiments can be formulated for oral administration or intravenous administration to a human subject or other administration or delivery method known in the pharmaceutical arts including for example, intramuscular, transdermal, nasal, buccal, and vaginal administration. In some embodiments that may be combined with any of the preceding embodiments, the tacrolimus can be administered in an amount to maintain a target blood level in a human subject of at least about 3 ng/ml blood for at least about 20 days after administering the third population of CD45⁺ cells, in an amount to maintain a target blood level of approximately 4 ng/ml or more for at least about 40 days after administering the third population of CD45⁺ cells, and/or in an amount that maintains a target blood level of approximately 4 ng/ml or more for at least about 40 days after administering the third population of CD45⁺ cells. In some embodiments that may be combined with any of the preceding embodiments, the tacrolimus is administered in an amount that maintains a target blood level of at most about 10 ng/ml for at least 30 days after administering the third population of CD45⁺ cells. In some embodiments that may be combined with any of the preceding embodiments, the tacrolimus is administered for at least about 60 days after administering the third population of CD45⁺ cells, for at least about 90 days after administering the third population of CD45⁺ cells, for at most about 150 days after administering the third population of CD45⁺ cells, for at most about 120 days after administering the third population of CD45⁺ cells. In some embodiments that may be combined with any of the preceding embodiments, the first population of CD45⁺ cells comprises at least about 0.5% granulocytes, at least about 1% granulocytes, at most about 5% granulocytes, at most about 3% granulocytes, at most about 3% monocytes, at most about 2% monocytes, at most about 0.5% lymphocytes, at most about 2% lymphocytes, at least about 15% granulocytes, at least about 20% granulocytes, at most about 35% granulocytes, at most about 30% granulocytes, at most about 25% granulocytes, at least about 15% monocytes, at least about 20% monocytes, at most about 35% monocytes, at most about 30% monocytes, at most about 25% monocytes, at least about 0.5% NK cells, and or at least about 2% NK cells. In some embodiments that may be combined with any of the preceding embodiments, the third population of CD45⁺ cells comprises at least about 0.1% CD34⁺ cells or from approximately 0.2% to approximately 20% CD34⁺ cells and/or at least about 0.1% Tregs. In some embodiments that may be combined with any of the preceding embodiments, the multi-component pharmaceutical treatment further comprises a conditioning regimen, wherein the conditioning regimen is administered before any of components (a) to (d) listed above, e.g., the conditioning regimen is administered from approximately two days to approximately ten days before any of (a) to (d). In some embodiments that may be combined with any of the preceding embodiments, the conditioning regimen is a myeloablative conditioning regimen. In some embodiments that may be combined with any of the preceding embodiments, the conditioning regimen comprises at least three conditioning reagents, wherein at least one conditioning reagent is thiotepa. In some embodiments that may be combined with any of the preceding embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In some embodiments that may be combined with any of the preceding embodiments, the conditioning regimen comprises one or more doses of busulfan, fludarabine and thiotepa. In some embodiments that may be combined with any of the preceding embodiments, the one or more doses comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively. In some embodiments that may be combined with any of the preceding embodiments, the first population of CD45⁺ cells comprises less than about 5 EU of endotoxins per ml of the solution, less than about 1 EU of endotoxins per ml of the solution, and/or less than about 0.5 EU of endotoxins per ml of the solution. In some embodiments that may be combined with any of the preceding embodiments, the population of cells enriched for Tregs comprises less than about 5 EU of endotoxins per ml of the solution, less than about 1 EU of endotoxins per ml of the solution, and/or less than about 0.5 EU of endotoxins per ml of the solution. In some embodiments that may be combined with any of the preceding embodiments, the third population of CD45⁺ cells comprises less than about 5 EU of endotoxins per ml of the solution, less than about 1 EU of endotoxins per ml of the solution, and/or less than about 0.5 EU of endotoxins per ml of the solution. In some embodiments that may be combined with any of the preceding embodiments, the HSPCs are CD34⁺. In some embodiments that may be combined with any of the preceding embodiments, the Tregs are CD4⁺ CD25⁺ CD127dim or CD4⁺ FOXP3⁺. In some embodiments that may be combined with any of the preceding embodiments, the population of cells enriched for Tregs comprises CD45⁺ cells, e.g., more than about 90% of the CD45⁺ cells are Tregs. In some embodiments that may be combined with any of the preceding embodiments, the population of cells enriched for Tregs comprises from approximately 1×10⁵ to approximately 1×10⁷ Tregs per kilogram of actual or ideal body weight of the human subject or from approximately 5×10⁵ to approximately 4×10⁶ Tregs per kilogram of actual or ideal body weight of the human subject. In some embodiments that may be combined with any of the preceding embodiments, the first population of CD45⁺ cells comprising HSPCs comprises from approximately 5×10⁵ to approximately 2×10⁷ HSPCs per kilogram of ideal body of the human subject. In some embodiments that may be combined with any of the preceding embodiments, the third population of CD45⁺ cells comprises from approximately 1×10⁵ to approximately 1×10⁷ Tcons per kilogram of actual or ideal body weight of the human subject or the third population of CD45⁺ cells comprises from approximately 5×10⁵ to approximately 5×10⁶ Tcons per kilogram of actual or ideal body weight of the human subject. In some embodiments that may be combined with any of the preceding embodiments, the first population of CD45⁺ cells and the population of cells enriched for Tregs are administered before the third population of CD45⁺ cells. In some embodiments that may be combined with any of the preceding embodiments, a first dose of the one or more doses of the GVHD prophylactic agent are administered after the administration of the third population of CD45⁺ cells.

Other aspects of the present disclosure relate to a method of treating a human subject diagnosed with a hematologic malignancy. In some embodiments, the method comprises administering to the human subject a solution comprising the first population of CD45⁺ cells, a solution comprising the population of cells enriched for regulatory Tregs, a solution comprising the third population of CD45⁺ cells, and a solution comprising one or more doses of the GVHD prophylactic agent. In some embodiments, the solution comprising the first population of CD45⁺ cells, the solution comprising the population of cells enriched for regulatory Tregs, the solution comprising the third population of CD45⁺ cells, and the solution comprising one or more doses of the GVHD prophylactic agent are as defined according to any herein disclosed multi-component pharmaceutical treatment.

In some embodiments that may be combined with any of the preceding embodiments, the hematologic malignancy may correspond to one or more of acute lymphocytic leukemia, acute myelogenous leukemia, chronic myelogenous leukemia, multiple myeloma, lymphoma, Hodgkin's lymphoma, non-Hodgkin lymphoma, myelodysplastic syndrome, myelofibrosis, and blastic plasmacytoid dendritic cell neoplasm (BPDCN). In some embodiments that may be combined with any of the preceding embodiments, administering comprises infusing into a human subject the first population of CD45⁺ cells, the population of cells enriched for Tregs, and the third population of CD45⁺ cells. In some embodiments that may be combined with any of the preceding embodiments, the third population of CD45⁺ cells can be administered at least about 12 hours after the first population of CD45⁺ cells, the third population of CD45⁺ cells is administered from approximately 24 to approximately 96 hours after the first population of CD45⁺ cells, the third population of CD45⁺ cells is administered from approximately 36 to approximately 60 hours after the first population of CD45⁺ cells, the third population of CD45⁺ cells is administered at least about 12 hours after the population of cells enriched for Tregs, the third population of CD45⁺ cells is administered from approximately 24 to approximately 96 hours after the population of cells enriched for Tregs, and/or the third population of CD45⁺ cells is administered from approximately 36 to approximately 60 hours after the population of cells enriched for Tregs. In some embodiments that may be combined with any of the preceding embodiments, the human subject does not develop higher than stage 2 GVHD within about 100 days of the administering of the third population of CD45⁺ cells, the human subject does not develop higher than stage 2 GVHD) within about 180 days or within about 200 days of the administering of the third population of CD45⁺ cells, the human subject does not develop higher than stage 2 GVHD within about 1 year of the administering of the third population of CD45⁺ cells. In some embodiments that may be combined with any of the preceding embodiments, the human subject has previously been or is concurrently treated for the hematologic malignancy. In some embodiments that may be combined with any of the preceding embodiments, the GVHD prophylactic agent is tacrolimus (and/or its analogues and derivatives) and is initially administered to the human subject at approximately 0.03 mg/kg of the human subject's actual or ideal body weight/day or the tacrolimus is initially administered from approximately 12 hours to approximately 24 hours after the administering of the third population of CD45⁺ cells. In some embodiments that may be combined with any of the preceding embodiments, a dose of the tacrolimus can be tapered starting at approximately 90 days after the first dose is administered to the human subject or a dose of the tacrolimus is tapered starting at approximately 45 days after the first dose is administered to the human subject. In some embodiments that may be combined with any of the preceding embodiments, the first population of CD45⁺ cells, the population of cells enriched for Tregs, and the third population of CD45⁺ cells are obtained from a single donor either on single day or over multiple days. In some embodiments that may be combined with any of the preceding embodiments, at least one mobilized peripheral blood donation is collected from a donor or at most two mobilized peripheral blood donations are collected from the donor. In some embodiments that may be combined with any of the preceding embodiments, at least one of the mobilized peripheral blood donations is processed and sorted to enrich CD34⁺ cells and Tregs. In some embodiments that may be combined with any of the preceding embodiments, the processing and sorting time of the one or more of the mobilized peripheral blood donations is less than about 35 hours, the processing and sorting time of the one or more of the mobilized peripheral blood donations is less than about 30 hours, the processing and sorting time of the one or more of the mobilized peripheral blood donations is less than about 25 hours, the processing and sorting time of the one or more of the mobilized peripheral blood donations is at most about 35 hours, and/or the processing and sorting time of the one or more of the mobilized peripheral blood donations is at most about 25 hours. In some embodiments that may be combined with any of the preceding embodiments, the one or more of the mobilized peripheral blood donations is processed and sorted using one or more immune-separation particles (ISPs), e.g., ISPs comprise affinity reagents such as immuno-magnetic separation particles which may be antibodies each conjugated to an iron-containing particle. In some embodiments, the affinity reagents comprise a plurality of CD34-reagents (e.g., an anti-CD34 antibody) that binds to one or more CD34 receptors on a HSPC. In some embodiments that may be combined with any of the preceding embodiments, an average number of ISPs per HSPC in the HSPC cell population is less than about 20,000, an average number of ISPs per HSPC in the HSPC cell population is equal to or less than about 10,000, and/or an average number of ISPs per HSPC in the HSPC cell population is from approximately 1500 to approximately 20,000. In some embodiments that may be combined with any of the preceding embodiments, the affinity reagents the affinity reagents comprise a plurality of CD25-reagents (e.g., an anti-CD25 antibody) that binds to one or more CD25 receptors on a Treg. In some embodiments that may be combined with any of the preceding embodiments, an average number of ISPs per T-reg cell in the Treg population is equal or less than about 4000 or an average number of ISPs per T-reg cell in the Treg population is from approximately 1500 to approximately 2500. In some embodiments that may be combined with any of the preceding embodiments, cells of the mobilized peripheral blood donation are sorted such that the first population of CD45⁺ cells comprises at most about 10% granulocytes. In some embodiments that may be combined with any of the preceding embodiments, cells of the mobilized peripheral blood donation are sorted such that the first population of CD45⁺ cells comprises at most about 7% granulocytes. In some embodiments that may be combined with any of the preceding embodiments, cells of the mobilized donor peripheral blood donation are sorted such that the first population of CD45⁺ cells comprises at most about 4% monocytes. In some embodiments that may be combined with any of the preceding embodiments, cells of the mobilized donor peripheral blood donation are sorted such that the first population of CD45⁺ cells comprises at least about 0.1% monocytes. In some embodiments that may be combined with any of the preceding embodiments, cells of the mobilized donor peripheral blood donation are sorted such that the population enriched for Tregs comprises at most about 10% CD25-cells. In some embodiments that may be combined with any of the preceding embodiments, the first population of CD45⁺ cells, the population of cells enriched for Tregs, and/or the third population of CD45⁺ cells is allogeneic relative to the human subject. In some embodiments that may be combined with any of the preceding embodiments, the first population of CD45⁺ cells, the population of cells enriched for Tregs, and/or the third population of CD45⁺ cells is obtained from a donor that is HLA-matched relative to the human subject. In some embodiments that may be combined with any of the preceding embodiments, the first population of CD45⁺ cells, the population of cells enriched for Tregs, and/or the third population of CD45⁺ cells is obtained from a donor that is HLA-mismatched relative to the human subject. In some embodiments that may be combined with any of the preceding embodiments, the first population of CD45⁺ cells, the population of cells enriched for Tregs, and/or the third population of CD45⁺ cells is obtained from a donor that is haploidentical relative to the human subject. In some embodiments that may be combined with any of the preceding embodiments, the third population of CD45⁺ cells comprises a population of invariant natural killer T cells (iNKTs), e.g., iNKTs that are CD3⁺Vα24Jα18⁺. In some embodiments that may be combined with any of the preceding embodiments, the population of iNKTs comprises more than about 5×10² iNKTs per kilogram of ideal body actual or ideal body weight of the human subject. In some embodiments that may be combined with any of the preceding embodiments, the population of iNKTs comprises from approximately 5×10² to approximately 1×10⁷ iNKTs per kilogram of ideal body actual or ideal body weight of the human subject. In some embodiments that may be combined with any of the preceding embodiments, the third population of CD45⁺ cells comprises a population of memory T cells (Tmems), e.g., Tmems that are CD3⁺ CD45RA⁻ CD45RO⁺. In some embodiments that may be combined with any of the preceding embodiments, the population of Tmems comprises more than about 3×10⁵ Tmems per kilogram of ideal body actual or ideal body weight of the human subject. In some embodiments that may be combined with any of the preceding embodiments, the population of Tmems comprises from approximately 3×10⁵ to approximately 1×10⁹ Tmems per kilogram of ideal body actual or ideal body weight of the human subject.

Other aspects of the present disclosure relate to a multi-component pharmaceutical treatment in which a risk and/or severity of an adverse event associated with the multi-component pharmaceutical treatment is reduced as compared to a similar pharmaceutical treatment in which a human subject receives Tcons but does not receive Tregs or is any herein-disclosed method in which a risk and/or severity of an adverse event associated with the method is reduced as compared to a similar method in which a human subject receives Tcons but does not receive Tregs.

In some embodiments that may be combined with any of the preceding embodiments, the adverse event is acute GVHD (aGVHD), which may include stage two or greater aGVHD. In some embodiments that may be combined with any of the preceding embodiments, the adverse event is chronic GVHD (cGVHD) which may be moderate to severe cGVHD. In some embodiments that may be combined with any of the preceding embodiments, the human subject has no cGVHD about one year after being administered the cell populations. In some embodiments that may be combined with any of the preceding embodiments, the adverse event is relapse of the human subject's malignancy. In some embodiments that may be combined with any of the preceding embodiments, the human subject has no relapse of their malignancy about one year after being administered the pharmaceutical dosing regimen. In some embodiments that may be combined with any of the preceding embodiments, the human subject has undergone myeloablative conditioning regimen before administration of any cell populations and the adverse event is associated with the myeloablative conditioning. In some embodiments that may be combined with any of the preceding embodiments, the method further comprises providing instructions for use (IFU), the IFU including instructions for administering the cell populations to the patient. In some embodiments that may be combined with any of the preceding embodiments, the IFU also include instructions for administering one or more pharmaceutical agents or compositions to the patient.

Other aspects of the present disclosure relate to a method of transplanting a conventional T cell (Tcons) population as a part of a treatment regimen for a hematologic malignancy in which the method reduces a risk and/or severity of an adverse event associated with the treatment regimen. In some embodiments, the method comprises administering to the patient a population of regulatory T cells (Tregs) comprising Tregs and a liquid suspending the Tregs; administering to the patient a heterogenous cell population comprising lymphocytes, granulocytes, monocytes and a liquid suspending the cells. In some embodiments, at least about 30% of the lymphocytes comprise Tcons. and after administration of the cell populations, the patient has a reduced risk and/or severity of the adverse event as compared to hematologic malignancy patients who received Tcons but did not receive Tregs.

Other aspects of the present disclosure relate to a method of transplanting cell populations into a human patient as a part of a treatment regimen for a hematologic malignancy in which the method reduces a risk and/or severity of an adverse event associated with the treatment regimen. In some embodiments, the method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending the cells. In some embodiments, at least about 30% of the lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

In some embodiments that may be combined with any of the preceding embodiments, the cell populations are administered to the patient by intravenous infusion. In some embodiments that may be combined with any of the preceding embodiments, the respective cell populations are provided as separate cell populations and are derived from a single human blood donor. In some embodiments that may be combined with any of the preceding embodiments, the adverse event is acute graft vs host disease (aGVHD), e.g., stage two or greater aGVHD. In some embodiments that may be combined with any of the preceding embodiments, the patient has no stage two or higher aGVHD about 180 days after being administered the cell populations. In some embodiments that may be combined with any of the preceding embodiments, the adverse event is chronic graft vs host disease (cGVHD). In some embodiments that may be combined with any of the preceding embodiments, the patient has no cGVHD about one year after being administered the cell populations. In some embodiments that may be combined with any of the preceding embodiments, the adverse event is moderate to severe cGVHD. In some embodiments that may be combined with any of the preceding embodiments, the patient does not have moderate to severe cGVHD about one year after being administered the cell populations. In some embodiments that may be combined with any of the preceding embodiments, the adverse event is relapse of the patient's malignancy. In some embodiments that may be combined with any of the preceding embodiments, the patient has no relapse of their malignancy about one year after being administered the cell populations. In some embodiments that may be combined with any of the preceding embodiments, the adverse event includes graft versus host disease (GVHD) and relapse of the patient's malignancy. In some embodiments that may be combined with any of the preceding embodiments, the patient has no GHVD or relapse of their malignancy one year after being administered the cell populations. In some embodiments that may be combined with any of the preceding embodiments, at least one of the cell populations comprise less than about 5 EU of endotoxins/ml of respective suspension liquid. In some embodiments that may be combined with any of the preceding embodiments, the patient has undergone myeloablative conditioning regimen before administration of the cell populations and the adverse event is associated with the myeloablative conditioning. In some embodiments that may be combined with any of the preceding embodiments, the adverse event includes relapse of the patient's malignancy or infection. In some embodiments that may be combined with any of the preceding embodiments, the heterogenous cell population comprises from approximately 0.2 to approximately 2.0 percent hematopoietic stem and progenitor cells. In some embodiments that may be combined with any of the preceding embodiments, the hematologic malignancy is leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, or blastic plasmacytoid dendritic cell neoplasm (BPDCN). In some embodiments that may be combined with any of the preceding embodiments, a genetic expression level of the T-reg cells correlates to cells that were harvested from the donor within about 60 hours prior to administration to the patient. In some embodiments that may be combined with any of the preceding embodiments, the number of T-reg cells in the T-reg population is about equal to the number of T-con cells in the heterogenous cell population. In some embodiments that may be combined with any of the preceding embodiments, the T-reg cells in the T-reg population inhibit activation of conventional T cells in the heterogenous cell population by the patient's healthy tissue by an amount up to approximately 20 percent In some embodiments that may be combined with any of the preceding embodiments, the peripheral blood of the patient exhibits an elevated ratio of Tregs to CD4⁺ T cells up to approximately 100 days after administration of the cell populations as compared to a healthy human subject that was not administered the cells populations. In some embodiments that may be combined with any of the preceding embodiments, at least about 50% of the cells in the HSPC's cell population are colony forming units. In some embodiments that may be combined with any of the preceding embodiments, at least one of the cell populations has an elevated amount of granulocyte colony-stimulating factor as compared to non-mobilized blood. In some embodiments that may be combined with any of the preceding embodiments, the at least one of the cell populations is the heterogenous cell population. In some embodiments that may be combined with any of the preceding embodiments, at least one of the cell populations have a plurality of immuno-separation particles (ISPs) attached to receptors on the cells of the cell population. In some embodiments that may be combined with any of the preceding embodiments, the plurality of ISPs are immuno-magnetic separation particles. In various embodiments, the plurality of ISPs comprise an antibody conjugated to an iron containing particle. In some embodiments that may be combined with any of the preceding embodiments, the population of Tcons is administered at least about 12 hours after the population of HSPCs, e.g., the population of Tcons is administered from approximately 24 to approximately 96 hours after the population of HSPCs or the population of Tcons is administered from approximately 36 to approximately 60 hours after the population of HSPCs. In some embodiments that may be combined with any of the preceding embodiments, the population of Tcons is administered at least about 12 hours after the population of cells comprising Tregs, e.g., the population of Tcons is administered from approximately 24 to approximately 96 hours after the population of cells comprising Tregs or the population of Tcons is administered from approximately 36 to approximately 60 hours after the population of cells comprising Tregs. In some embodiments that may be combined with any of the preceding embodiments, a herein-disclosed method further comprises administering to the patient over a period of time up to approximately 180 days a single graft versus host disease (GVHD) prophylactic agent (GVHDPA) comprising tacrolimus (tacrolimus GHVDPA); wherein the tacrolimus GHVDPA is administered to maintain a concentration of tacrolimus in the patient's blood above a threshold level during the period of time; and wherein a risk and/or severity of GHVD is significantly reduced. In some embodiments that may be combined with any of the preceding embodiments, the threshold level is above about 4 ng of tacrolimus per ml of patient blood or the threshold level is above about 5 ng of tacrolimus per ml of patient blood. In some embodiments that may be combined with any of the preceding embodiments, the tacrolimus GHVDPA is administered to maintain a concentration of tacrolimus in the patients' blood below an upper threshold level during the period of time. In some embodiments that may be combined with any of the preceding embodiments, the upper threshold level is below about 10 ng of tacrolimus per ml of patient blood. In some embodiments that may be combined with any of the preceding embodiments, the patient has a reduced risk of at least one of malignancy relapse, infection, or renal failure. In some embodiments that may be combined with any of the preceding embodiments, the patient does not develop GVHD within about 30 days of administration of the Tcons, does not develop GVHD within about 100 days of administration of the Tcons, does not develop GVHD within about 180 days of administration of the Tcons, and/or does not develop GVHD within about one year of administration of the Tcons. In some embodiments that may be combined with any of the preceding embodiments, the tacrolimus graft versus host disease (GVHD) prophylactic agent (GVHDPA) may be intravenously administered or orally administered to the patient. In some embodiments that may be combined with any of the preceding embodiments, administration of the tacrolimus graft versus host disease (GVHD) prophylactic agent (GVHDPA) is started from approximately 12 to approximately 24 hours after administration of the T-cons. In some embodiments that may be combined with any of the preceding embodiments, the tacrolimus GHVDPA is administered for a period of time up to approximately 90 days, is administered for a period of time up to approximately 60 days. In some embodiments that may be combined with any of the preceding embodiments, the tacrolimus GHVDPA is initially administered to the patient at approximately 0.03 mg/kg patient's actual or ideal body weight/day. In some embodiments that may be combined with any of the preceding embodiments, a dose of the tacrolimus GVHDPA administered to the patient is tapered starting at approximately 90 days after a first dose is administered to the patient or is tapered starting at approximately 45 days after a first dose is administered to the patient. In some embodiments that may be combined with any of the preceding embodiments, the method further comprises administering a myeloablative conditioning regimen to the patient prior to the administration of any cell population, the conditioning regimen comprising administration of at least one conditioning agent to the patient. In some embodiments that may be combined with any of the preceding embodiments, the patient does not receive any irradiation as part of the myeloablative conditioning regimen. In some embodiments that may be combined with any of the preceding embodiments, the at least one conditioning agent is administered from approximately two to approximately ten days prior to the administration of any of the cell populations. In some embodiments that may be combined with any of the preceding embodiments, the at least one conditioning agent is administered about five days prior to the administration of any of the cell populations. In some embodiments that may be combined with any of the preceding embodiments, the at least one conditioning agent comprises thiotepa. In some embodiments that may be combined with any of the preceding embodiments, a dose of thiotepa administered to the patient is in a range of from approximately 5 to approximately 10 mg per kilogram of actual or ideal body weight. In some embodiments that may be combined with any of the preceding embodiments, the at least one conditioning agent comprises busulfan and fludarabine. In some embodiments that may be combined with any of the preceding embodiments, doses of thiotepa, busulfan, and fludarabine administered to the patient comprise about 10 mg per kilogram of the patient's actual or ideal body weight, about 9.6 mg per kilogram of the patient's actual or ideal body weight, and approximately 150 mg per meter² body surface area respectively. In some embodiments that may be combined with any of the preceding embodiments, the method further comprises providing instructions for use (IFU), the IFU including instructions for administering the cell populations to the patient. In some embodiments that may be combined with any of the preceding embodiments, the IFU also include instructions for administering one or more pharmaceutical agents or compositions to the patient.

Other aspects of the present disclosure relate to a method of transplanting cell populations into a human patient as a part of a treatment regimen for a hematologic malignancy. In some embodiments, the method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

Other aspects of the present disclosure relate to a kit that comprises a solution comprising a first container comprising a first population of CD45⁺ cells, a second container comprising a solution comprising a third population of CD45⁺ cells, and a third container comprising a solution comprising a population of cells enriched for regulatory T cells (Tregs). In some embodiments, the solution comprising the first population of CD45⁺ cells, the solution comprising the third population of CD45⁺ cells, and the solution comprising the population of cells enriched for Tregs are as defined according to any herein disclosed multi-component pharmaceutical treatment or method. In some embodiments, the kit further comprises a fourth container comprising the GVHD prophylactic agent. In some embodiments that may be combined with any of the preceding embodiments, the further comprising instructions for performing any herein-disclosed method.

Other aspects of the present disclosure relate to a kit comprising: (a) one or more reagents to sort CD34⁺ cells from a mobilized peripheral blood composition; (b) one or more reagents to sort regulatory T cells (Tregs) from the mobilized peripheral blood composition; (c) one or more reagents to detect a number of CD3⁺ conventional T cells in the mobilized peripheral blood; and (d) a solution comprising one or more doses of a graft vs host disease (GVHD) prophylactic agent. In some embodiments, the kit further comprises instructions for performing any herein-disclosed method.

Other aspects of the present disclosure relate to a method of transplanting a conventional T cell (Tcon) population into a human subject without eliciting a stage 2 or higher graft versus host disease (GVHD) response up to approximately 100 days after transplanting. In some embodiments, the method comprises (i) administering a heterogenous cell population comprising lymphocytes, granulocytes, and monocytes, wherein at least about 30% of the lymphocytes comprises conventional T cells (Tcons); and (ii). administering a population of regulatory T cells (Tregs). In some embodiments, the heterogenous cell component and/or the population of Tregs comprise less than about 5 EU/ml endotoxins.

Other aspects of the present disclosure relate to a method of treating a human subject comprising a step (a) of administering a plurality of populations of cells, in which the plurality of populations of cells comprises: (i). a population of hematopoietic stem and progenitor cells (HSPCs); (ii) a population of cells comprising regulatory T cells (Tregs); and (iii) a population of conventional T cells (Tcons); and a step (b) of administering no more than one graft versus host disease (GVHD) prophylactic agent for less than about 120 days. In some embodiments, the population of HSPCs comprises less than about 2% CD3⁺ cells.

Other aspects of the present disclosure relate to a method treating a human subject in need thereof comprising administering to the human subject at least two pharmaceutical compositions, wherein the pharmaceutical compositions are selected from (a) a pharmaceutical composition comprising a population of hematopoietic stem and progenitor cells (HSPCs); (b) a pharmaceutical composition comprising a population of regulatory T cells (Tregs); and (c) a pharmaceutical composition comprising a population of conventional T cells (Tcons). In some embodiments, the pharmaceutical compositions (a), (b) and (c) comprise less than about 5 EU/ml endotoxins each; and less than 15 human subjects in a group of 100 human subjects administered the two or more pharmaceutical compositions develops a stage 2 or higher graft versus host disease (GVHD) response within about 30 days after being administered the pharmaceutical composition comprising the population of Tcons.

Other aspects of the present disclosure relate to a method of transplanting a conventional T cell (Tcon) population into a human subject without eliciting a stage 2 or higher graft versus host disease (GVHD) response up to approximately 100 days after transplanting. In some embodiments, the method comprises: (i). administering a solution comprising a population of conventional T cells (Tcons); and (ii). administering a solution comprising a population of regulatory T cells (Tregs). In some embodiments, the population of Tcons is cryopreserved for at least about 4 hours; and the solution comprising the population of Tcons and the solution comprising the population of Tregs comprise less than about 5 EU of endotoxins per ml of the solution.

Other aspects of the present disclosure relate to a method of treating a hematologic malignancy in a human subject in need thereof, the method comprising administering to the human subject: (a) a population of hematopoietic stem and progenitor cells (HSPCs); (b) a population of regulatory T cells (Tregs); and (c) a population of conventional T cells (Tcons). In some embodiments, the population of HSPCs and the population of Tregs are administered prior to the population of Tcons; and peripheral blood of the human subject exhibits an elevated Treg count until about 100 days after the administering the three populations of cells as compared to a healthy human subject that was not administered the three populations of cells.

Other aspects of the present disclosure relate to a method of transplanting a conventional T cell (Tcon) population into a human subject without eliciting a stage 2 or higher graft versus host disease (GVHD) response up to approximately 100 days after transplanting. In some embodiments, the method comprises (i). administering a population of conventional T cells (Tcons); and (ii). administering a population of regulatory T cells (Tregs). In some embodiments, the population of Tcons is administered at least about 12 hours after the population of Tregs is administered; and the population of Tcons and the population of Tregs comprise less than about 5 EU/ml endotoxins.

Certain aspects of the present disclosure relate to a method of treating a human subject diagnosed with a hematologic malignancy, the method comprising administering to the human subject a multi-component pharmaceutical treatment comprising: (a) a solution comprising a first population of CD45⁺ cells comprising hematopoietic stem and progenitor cells (HSPCs); (b) a solution comprising a population of cells enriched for regulatory T cells (Tregs); (c) a solution comprising a third population of CD45⁺ cells wherein the third population of CD45⁺ cells comprise CD3⁺ conventional T cells (Tcons); and (d) a solution comprising one or more doses of a graft vs host disease (GVHD) prophylactic agent, wherein the first population of CD45⁺ cells, the population of cells enriched for Tregs, and the third population of CD45⁺ cells are obtained from a single allogeneic donor that has at least one HLA mismatch relative to the human subject, and wherein incidence of non-relapse mortality of the human subject is decreased after administration and/or overall survival of the human subject is increased after administration, relative to a corresponding human subject that has been administered a standard myeloablative allogeneic hematopoietic stem cell transplant (alloHSCT) from a donor that has at least one HLA mismatch relative to the corresponding human subject.

In some embodiments, the hematologic malignancy is selected from: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN). In some embodiments that may be combined with any of the preceding embodiments, the administering comprises infusing into the human subject the first population of CD45⁺ cells, the population of cells enriched for Tregs, and the third population of CD45⁺ cells. In some embodiments that may be combined with any of the preceding embodiments, the third population of CD45⁺ cells is administered at least about 12 hours after the first population of CD45⁺ cells. In some embodiments that may be combined with any of the preceding embodiments, the third population of CD45⁺ cells is administered from approximately 24 to approximately 120 hours after the first population of CD45⁺ cells. In some embodiments that may be combined with any of the preceding embodiments, the third population of CD45⁺ cells is administered from approximately 36 to approximately 72 hours after the first population of CD45⁺ cells. In some embodiments that may be combined with any of the preceding embodiments, the third population of CD45⁺ cells is administered at least about 12 hours after the population of cells enriched for Tregs. In some embodiments that may be combined with any of the preceding embodiments, the third population of CD45⁺ cells is administered from approximately 24 to approximately 120 hours after the population of cells enriched for Tregs. In some embodiments that may be combined with any of the preceding embodiments, the third population of CD45⁺ cells is administered from approximately 36 to approximately 72 hours after the population of cells enriched for Tregs. In some embodiments that may be combined with any of the preceding embodiments, the HSPCs are CD34⁺. In some embodiments that may be combined with any of the preceding embodiments, the first population of CD45⁺ cells comprises from approximately 5×10⁵ to approximately 2×10⁷ HSPCs per kilogram of actual or ideal body weight of the human subject. In some embodiments that may be combined with any of the preceding embodiments, the Tregs are CD4⁺ CD25⁺ CD127dim. In some embodiments that may be combined with any of the preceding embodiments, the population of cells enriched for Tregs comprises CD45⁺ cells, wherein more than about 90% of said CD45⁺ cells are Tregs. In some embodiments that may be combined with any of the preceding embodiments, the Tregs are CD4⁺ CD25⁺ CD127dim or CD4⁺ FOXP3⁺. In some embodiments that may be combined with any of the preceding embodiments, the population of cells enriched for Tregs comprises from approximately 1×10⁵ to approximately 1×10⁷ Tregs per kilogram of actual or ideal body weight of the human subject. In some embodiments that may be combined with any of the preceding embodiments, the third population of CD45⁺ cells comprises from approximately 1×10⁵ to approximately 1×10⁷ Tcons per kilogram of actual or ideal body weight of said human subject. In some embodiments that may be combined with any of the preceding embodiments, the human subject does not develop higher than stage 2 GVHD within about 100 days of the administering of the third population of CD45⁺ cells. In some embodiments that may be combined with any of the preceding embodiments, the human subject does not develop higher than stage 2 GVHD within about 180 days or within about 200 days of the administering of the third population of CD45⁺ cells. In some embodiments that may be combined with any of the preceding embodiments, the human subject does not develop higher than stage 2 GVHD within about 1 year of the administering of the third population of CD45⁺ cells. In some embodiments that may be combined with any of the preceding embodiments, the human subject has previously been or is concurrently being treated for the hematologic malignancy. In some embodiments that may be combined with any of the preceding embodiments, the GVHD prophylactic agent is tacrolimus and is initially administered to the human subject at approximately 0.03 mg/kg human subject's actual or ideal body weight/day. In some embodiments that may be combined with any of the preceding embodiments, the GVHD prophylactic agent is initially administered from approximately 12 hours to approximately 24 hours after the administering of the third population of CD45⁺ cells. In some embodiments that may be combined with any of the preceding embodiments, the tacrolimus is administered in an amount to maintain a target blood level of at least about 3 ng/ml for at least about 20 days after administering the third population of CD45⁺ cells. In some embodiments that may be combined with any of the preceding embodiments, the tacrolimus is administered in an amount that maintains a target blood level of approximately 4 ng/ml or more for at least about 40 days after administering the third population of CD45⁺ cells. In some embodiments that may be combined with any of the preceding embodiments, the tacrolimus is administered for at least about 60 days, at least about 90 days, at least about 120 days, or at most about 160 days after administering the third population of CD45⁺ cells. In some embodiments that may be combined with any of the preceding embodiments, administration of the GVHD prophylactic agent is tapered starting at approximately 90 days after initial administration of the GVHD prophylactic agent. In some embodiments that may be combined with any of the preceding embodiments, the method further comprises administering about 1000 mg of mycophenolate mofetil (MMF). In some embodiments that may be combined with any of the preceding embodiments, the MMF is initially administered from approximately 12 hours to approximately 24 hours after the administering of the third population of CD45⁺ cells. In some embodiments that may be combined with any of the preceding embodiments, administration of the MMF is tapered starting at approximately 30 days, at approximately 35 days, at approximately 40 days, at approximately 41 days, at approximately 42 days, at approximately 43 days, at approximately 44 days, at approximately 45 days, at approximately 46 days, at approximately 47 days, at approximately 48 days, at approximately 49 days, or at approximately 50 days after initial administration of the MMF. In some embodiments that may be combined with any of the preceding embodiments, the single allogeneic donor that has at least one HLA mismatch is unrelated to the human subject. In some embodiments that may be combined with any of the preceding embodiments, the single allogeneic donor that has at least one HLA mismatch is related to the human subject. In some embodiments that may be combined with any of the preceding embodiments, the method further comprises collecting one or more, or two or more mobilized peripheral blood donations from the donor. In some embodiments that may be combined with any of the preceding embodiments, the method further comprises collecting at most two mobilized peripheral blood donations from the donor. In some embodiments that may be combined with any of the preceding embodiments, the peripheral blood donations is mobilized by granulocyte colony-stimulating factor (G-CSF), granulocyte macrophage colony-stimulating factor (GM-CSF), stem cell factor (SCF), a SDF-1 antagonist, a CXCR4 antagonist (e.g., POL6326, BKT-140, TG-0054, NOX-A12), MOZOBIL® (plerixafor), a CXCR2 ligand (e.g., GROβ), a sphingosine-1-phosphatase (S1P) agonist. (e.g., SEW2871), a VCAM/VLA-4 inhibitor (e.g., BIO5192), a proteosome inhibitor (e.g., Bortezomib), parathyroid hormone, a hypoxia inducible factor (HIF) stabilizer (e.g., FG-4497), and combinations thereof. In some embodiments that may be combined with any of the preceding embodiments, at least one of the mobilized peripheral blood donations is processed and sorted using one or more immune-separation particles (ISPs) to enrich CD34⁺ cells and Tregs. In some embodiments that may be combined with any of the preceding embodiments, the one or more ISPs comprise affinity reagents, optionally wherein the affinity reagents are immuno-magnetic separation particles, optionally wherein the immuno-magnetic separation particles are antibodies each conjugated to an iron-containing particle. In some embodiments that may be combined with any of the preceding embodiments, an average number of ISP's per HSPC in the HSPC cell population is equal to or less than about 20,000. In some embodiments that may be combined with any of the preceding embodiments, an average number of ISP's per HSPC in the HSPC cell population is from approximately 1000 to approximately 20,000. In some embodiments that may be combined with any of the preceding embodiments, the affinity reagents comprise a plurality of CD25-reagents that binds to one or more CD25 receptors on a Treg. In some embodiments that may be combined with any of the preceding embodiments, an average number of ISPs per T-reg cell in the Treg population is equal or less than about 4000. In some embodiments that may be combined with any of the preceding embodiments, an average number of ISPs per T-reg cell in the Treg population is from approximately 1500 to approximately 2500. In some embodiments that may be combined with any of the preceding embodiments, the at least one HLA mismatch is at an allele selected from: HLA-A, HLA-B, HLA-C, DRB-1, and any combination thereof. In some embodiments that may be combined with any of the preceding embodiments, the allogeneic donor that has at least one HLA mismatch is 6/8 HLA-mismatched relative to the human subject or is 7/8 HLA-mismatched relative to the human subject. In some embodiments that may be combined with any of the preceding embodiments, the allogeneic donor is 7/8 HLA-mismatched relative to the human subject. In some embodiments that may be combined with any of the preceding embodiments, the allogeneic donor that is 7/8 HLA-mismatched relative to the human subject has a mismatch in HLA-A. In some embodiments that may be combined with any of the preceding embodiments, the allogeneic donor that is 7/8 HLA-mismatched relative to the human subject has a mismatch in HLA-B. In some embodiments that may be combined with any of the preceding embodiments, the allogeneic donor that is 7/8 HLA-mismatched relative to the human subject has a mismatch in HLA-C. In some embodiments that may be combined with any of the preceding embodiments, the allogeneic donor that is 7/8 HLA-mismatched relative to the human subject has a mismatch in HLA-DRB1. In some embodiments that may be combined with any of the preceding embodiments, the allogeneic donor that has at least one HLA mismatch relative to the human subject has a mismatched HLA allele as a result of the allogeneic donor being homozygous for the HLA allele while the human subject is heterogeneous for the HLA allele. In some embodiments that may be combined with any of the preceding embodiments, the allogeneic donor that has at least one HLA mismatch relative to the human subject has a mismatched HLA allele as a result of the allogeneic donor being heterozygous for the HLA allele while the human subject is homozygous for the HLA allele. In some embodiments that may be combined with any of the preceding embodiments, the allogeneic donor that has at least one HLA mismatch relative to the human subject has a mismatched HLA allele as a result of both the allogeneic donor and the human subject being heterozygous for the HLA allele. In some embodiments that may be combined with any of the preceding embodiments, the method further comprises a conditioning regimen, wherein the conditioning regimen is administered before any of (a) to (d). In some embodiments that may be combined with any of the preceding embodiments, the conditioning regimen is administered from approximately two days to approximately ten days before any of (a) to (d). In some embodiments that may be combined with any of the preceding embodiments, the conditioning regimen is a myeloablative conditioning regimen. In some embodiments that may be combined with any of the preceding embodiments, the myeloablative conditioning regimen comprises at least three conditioning reagents, wherein at least one conditioning reagent comprises thiotepa. In some embodiments that may be combined with any of the preceding embodiments, the myeloablative conditioning regimen comprises one or more doses of busulfan, fludarabine and thiotepa. In some embodiments that may be combined with any of the preceding embodiments, the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.

Other aspects of the present disclosure relate to a multi-component pharmaceutical treatment to be administered to a human subject in need thereof, the multi-component treatment comprising: (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent, wherein the first population of CD45⁺ cells, the population of cells enriched for Tregs, and the third population of CD45⁺ cells have at least one HLA mismatch relative to the human subject, and wherein upon administration, the first population of CD45⁺ cells, the population of cells enriched for Tregs, and the third population of CD45⁺ cells decrease incidence of non-relapse mortality in the human subject and/or increase overall survival in human subject compared to incidence of non-relapse mortality and/or over survival in a corresponding human subject that has been administered a standard myeloablative allogeneic hematopoietic stem cell transplant (alloHSCT) that has at least one HLA mismatch relative to the corresponding human subject.

In some embodiments that may be combined with any of the preceding embodiments, the GVHD prophylactic agent is tacrolimus. In some embodiments that may be combined with any of the preceding embodiments, the one or more doses of the GVHD prophylactic agent comprises tacrolimus in an amount that maintains a target blood level of at least about 3 ng/ml for at least about 20 days after administration of the third population of CD45⁺ cells. In some embodiments that may be combined with any of the preceding embodiments, the treatment further comprises a conditioning regimen. In some embodiments that may be combined with any of the preceding embodiments, the conditioning regimen is a myeloablative conditioning regimen. In some embodiments that may be combined with any of the preceding embodiments, the myeloablative conditioning regimen comprises at least three conditioning reagents, wherein at least one conditioning reagent comprises thiotepa. In some embodiments that may be combined with any of the preceding embodiments, the myeloablative conditioning regimen comprises one or more doses of busulfan, fludarabine and thiotepa comprising from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively. In some embodiments that may be combined with any of the preceding embodiments, the HSPCs are CD34⁺. In some embodiments that may be combined with any of the preceding embodiments, the Tregs are CD4⁺ CD25⁺ CD127dim. In some embodiments that may be combined with any of the preceding embodiments, the population of cells enriched for Tregs comprises CD45⁺ cells, wherein more than about 90% of the CD45⁺ cells are Tregs. In some embodiments that may be combined with any of the preceding embodiments, the Tregs are CD4⁺ CD25⁺ CD127dimor CD4⁺ FOXP3⁺. In some embodiments that may be combined with any of the preceding embodiments, the first population of CD45⁺ cells comprising HSPCs comprises from approximately 5×10⁵ to approximately 2×10⁷ HSPCs per kilogram of actual or ideal body weight of said human subject. In some embodiments that may be combined with any of the preceding embodiments, the population of cells enriched for Tregs comprises from approximately 1×10⁵ to approximately 1×10⁷ Tregs per kilogram of actual or ideal body weight of said human subject. In some embodiments that may be combined with any of the preceding embodiments, the third population of CD45⁺ cells comprises from approximately 1×10⁵ to approximately 1×10⁷ Tcons per kilogram of actual or ideal body weight of said human subject. In some embodiments that may be combined with any of the preceding embodiments, the first population of CD45⁺ cells and the population of cells enriched for Tregs are formulated for administration prior to administration of the third population of CD45⁺ cells. In some embodiments that may be combined with any of the preceding embodiments, a first dose of the one or more doses of the GVHD prophylactic agent is formulated for administration subsequent to administration of the third population of CD45⁺ cells. In some embodiments that may be combined with any of the preceding embodiments, the treatment further comprises about 1000 mg of mycophenolate mofetil (MMF). In some embodiments that the MMF is formulated for may be combined with any of the preceding embodiments, administration from approximately 12 hours to approximately 24 hours subsequent to administering the third population of CD45⁺ cells. In some embodiments that may be combined with any of the preceding embodiments, the single allogeneic donor that has at least one HLA mismatch is unrelated to the human subject. In some embodiments that may be combined with any of the preceding embodiments, the single allogeneic donor that has at least one HLA mismatch is related to the human subject. In some embodiments that may be combined with any of the preceding embodiments, the at least one HLA mismatch is at an allele selected from: HLA-A, HLA-B, HLA-C, DRB-1, and any combination thereof. In some embodiments that may be combined with any of the preceding embodiments, the allogeneic donor that has at least one HLA mismatch is 6/8 HLA-mismatched relative to the human subject or is 7/8 HLA-mismatched relative to the human subject. In some embodiments that may be combined with any of the preceding embodiments, the allogeneic donor is 7/8 HLA-mismatched relative to the human subject. In some embodiments that may be combined with any of the preceding embodiments, the allogeneic donor that is 7/8 HLA-mismatched relative to the human subject has a mismatch in HLA-A. In some embodiments that may be combined with any of the preceding embodiments, the allogeneic donor that is 7/8 HLA-mismatched relative to the human subject has a mismatch in HLA-B. In some embodiments that may be combined with any of the preceding embodiments, the allogeneic donor that is 7/8 HLA-mismatched relative to the human subject has a mismatch in HLA-C. In some embodiments that may be combined with any of the preceding embodiments, the allogeneic donor that is 7/8 HLA-mismatched relative to the human subject has a mismatch in HLA-DRB1. In some embodiments that may be combined with any of the preceding embodiments, the allogeneic donor that has at least one HLA mismatch relative to the human subject has a mismatched HLA allele as a result of the allogeneic donor being homozygous for the HLA allele while the human subject is heterogeneous for the HLA allele. In some embodiments that may be combined with any of the preceding embodiments, the allogeneic donor that has at least one HLA mismatch relative to the human subject has a mismatched HLA allele as a result of the allogeneic donor being heterozygous for the HLA allele while the human subject is homozygous for the HLA allele. In some embodiments that may be combined with any of the preceding embodiments, the allogeneic donor that has at least one HLA mismatch relative to the human subject has a mismatched HLA allele as a result of both the allogeneic donor and the human subject being heterozygous for the HLA allele.

Other aspects of the present disclosure relate to a method of treating a human subject diagnosed with a hematologic malignancy, the method comprising administering to the human subject a multi-component pharmaceutical treatment comprising: (a) a myeloablative conditioning regimen comprising one or more doses of busulfan, fludarabine and thiotepa; (b) a solution comprising a first population of CD45⁺ cells comprising hematopoietic stem and progenitor cells (HSPCs); (c) a solution comprising a population of cells enriched for regulatory T cells (Tregs); (d) a solution comprising a third population of CD45⁺ cells wherein the third population of CD45⁺ cells comprise CD3⁺ conventional T cells (Tcons); and (e) a solution comprising one or more doses of a graft vs host disease (GVHD) prophylactic agent, wherein the conditioning regimen is administered from approximately two days to approximately ten days before any of (b) to (e). In some embodiments that may be combined with any of the preceding embodiments, the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively. In some embodiments that may be combined with any of the preceding embodiments, GVHD and relapse-free survival (GFRS) of the human subject increases by at least about 3.5-fold after administration, overall survival increased by at least about 1.25-fold after administration, and/or incidence of non-relapse mortality decreases by at least about 60% after administration, relative to a corresponding human subject that has been administered a standard myeloablative allogeneic hematopoietic stem cell transplant (alloHSCT).

Other aspects of the present disclosure relate to a method of treating a human subject diagnosed with a hematologic malignancy, the method comprising administering to the human subject a multi-component pharmaceutical treatment comprising: (a) a solution comprising a first population of CD45⁺ cells comprising hematopoietic stem and progenitor cells (HSPCs); (b) a solution comprising a population of cells enriched for regulatory T cells (Tregs); (c) a solution comprising a third population of CD45⁺ cells wherein the third population of CD45⁺ cells comprise CD3⁺ conventional T cells (Tcons); and (d) a solution comprising one or more doses of a graft vs host disease (GVHD) prophylactic agent, wherein GVHD and relapse-free survival (GFRS) of the human subject increases by at least about 3.5-fold after administration, overall survival increased by at least about 1.25-fold after administration, and/or incidence of non-relapse mortality decreases by at least about 60% after administration, relative to a corresponding human subject that has been administered a standard myeloablative allogeneic hematopoietic stem cell transplant (alloHSCT). In some embodiments that may be combined with any of the preceding embodiments, the multi-component pharmaceutical treatment further comprises a myeloablative conditioning regimen comprising one or more doses of busulfan, fludarabine and thiotepa. In some embodiments that may be combined with any of the preceding embodiments, the conditioning regimen is administered from approximately two days to approximately ten days before any of (a) to (d). In some embodiments that may be combined with any of the preceding embodiments, the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.

In some embodiments that may be combined with any of the preceding embodiments, the hematologic malignancy is selected from: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN). In some embodiments that may be combined with any of the preceding embodiments, the administering comprises infusing into the human subject the first population of CD45⁺ cells, the population of cells enriched for Tregs, and the third population of CD45⁺ cells. In some embodiments that may be combined with any of the preceding embodiments, the third population of CD45⁺ cells is administered at least about 12 hours after the first population of CD45⁺ cells. In some embodiments that may be combined with any of the preceding embodiments, the third population of CD45⁺ cells is administered from approximately 24 to approximately 120 hours after the first population of CD45⁺ cells. In some embodiments that may be combined with any of the preceding embodiments, the third population of CD45⁺ cells is administered from approximately 36 to approximately 72 hours after the first population of CD45⁺ cells. In some embodiments that may be combined with any of the preceding embodiments, the third population of CD45⁺ cells is administered at least about 12 hours after the population of cells enriched for Tregs. In some embodiments that may be combined with any of the preceding embodiments, the third population of CD45⁺ cells is administered from approximately 24 to approximately 120 hours after the population of cells enriched for Tregs. In some embodiments that may be combined with any of the preceding embodiments, the third population of CD45⁺ cells is administered from approximately 36 to approximately 72 hours after the population of cells enriched for Tregs. In some embodiments that may be combined with any of the preceding embodiments, the HSPCs are CD34⁺. In some embodiments that may be combined with any of the preceding embodiments, the first population of CD45⁺ cells comprises from approximately 5×10⁵ to approximately 2×10⁷ HSPCs per kilogram of actual or ideal body weight of the human subject. In some embodiments that may be combined with any of the preceding embodiments, the Tregs are CD4⁺ CD25⁺ CD127dim. In some embodiments that may be combined with any of the preceding embodiments, the population of cells enriched for Tregs comprises CD45⁺ cells, wherein more than about 90% of said CD45⁺ cells are Tregs. In some embodiments that may be combined with any of the preceding embodiments, the Tregs are CD4⁺ CD25⁺ CD127dimor CD4⁺ FOXP3⁺. In some embodiments that may be combined with any of the preceding embodiments, the population of cells enriched for Tregs comprises from approximately 1×10⁵ to approximately 1×10⁷ Tregs per kilogram of actual or ideal body weight of the human subject. In some embodiments that may be combined with any of the preceding embodiments, the third population of CD45⁺ cells comprises from approximately 1×10⁵ to approximately 1×10⁷ Tcons per kilogram of actual or ideal body weight of said human subject. In some embodiments that may be combined with any of the preceding embodiments, the human subject does not develop higher than stage 2 GVHD within about 100 days of the administering of the third population of CD45⁺ cells. In some embodiments that may be combined with any of the preceding embodiments, the human subject does not develop higher than stage 2 GVHD within about 180 days or within about 200 days of the administering of the third population of CD45⁺ cells. In some embodiments that may be combined with any of the preceding embodiments, the human subject does not develop higher than stage 2 GVHD within about 1 year of the administering of the third population of CD45⁺ cells. In some embodiments that may be combined with any of the preceding embodiments, the human subject has previously been or is concurrently being treated for the hematologic malignancy. In some embodiments that may be combined with any of the preceding embodiments, the GVHD prophylactic agent is tacrolimus and is initially administered to the human subject at approximately 0.03 mg/kg human subject's actual or ideal body weight/day. In some embodiments that may be combined with any of the preceding embodiments, the GVHD prophylactic agent is initially administered from approximately 12 hours to approximately 24 hours after the administering of the third population of CD45⁺ cells. In some embodiments that may be combined with any of the preceding embodiments, the tacrolimus is administered in an amount to maintain a target blood level of at least about 3 ng/ml for at least about 20 days after administering the third population of CD45⁺ cells. In some embodiments that may be combined with any of the preceding embodiments, the tacrolimus is administered in an amount that maintains a target blood level of approximately 4 ng/ml or more for at least about 40 days after administering the third population of CD45⁺ cells. In some embodiments that may be combined with any of the preceding embodiments, the tacrolimus is administered for at least about 60 days, at least about 90 days, at least about 120 days, or at most about 160 days after administering the third population of CD45⁺ cells. In some embodiments that may be combined with any of the preceding embodiments, administration of the GVHD prophylactic agent is tapered starting at approximately 90 days after initial administration of the GVHD prophylactic agent. In some embodiments that may be combined with any of the preceding embodiments, the method further comprises collecting one or more, or two or more mobilized peripheral blood donations from the donor. In some embodiments that may be combined with any of the preceding embodiments, the method further comprises collecting at most two mobilized peripheral blood donations from the donor. In some embodiments that may be combined with any of the preceding embodiments, the peripheral blood donations is mobilized by granulocyte colony-stimulating factor (G-CSF), granulocyte macrophage colony-stimulating factor (GM-CSF), stem cell factor (SCF), a SDF-1 antagonist, a CXCR4 antagonist (e.g., POL6326, BKT-140, TG-0054, NOX-A12), MOZOBIL® (plerixafor), a CXCR2 ligand (e.g., GROβ), a sphingosine-1-phosphatase (S1P) agonist. (e.g., SEW2871), a VCAM/VLA-4 inhibitor (e.g., BIO5192), a proteosome inhibitor (e.g., Bortezomib), parathyroid hormone, a hypoxia inducible factor (HIF) stabilizer (e.g., FG-4497), and combinations thereof. In some embodiments that may be combined with any of the preceding embodiments, at least one of the mobilized peripheral blood donations is processed and sorted using one or more immune-separation particles (ISPs) to enrich CD34⁺ cells and Tregs. In some embodiments that may be combined with any of the preceding embodiments, the one or more ISPs comprise affinity reagents, optionally wherein the affinity reagents are immuno-magnetic separation particles, optionally wherein the immuno-magnetic separation particles are antibodies each conjugated to an iron-containing particle. In some embodiments that may be combined with any of the preceding embodiments, an average number of ISP's per HSPC in the HSPC cell population is equal to or less than about 20,000. In some embodiments that may be combined with any of the preceding embodiments, an average number of ISP's per HSPC in the HSPC cell population is from approximately 1000 to approximately 20,000. In some embodiments that may be combined with any of the preceding embodiments, the affinity reagents comprise a plurality of CD25-reagents that binds to one or more CD25 receptors on a Treg. In some embodiments that may be combined with any of the preceding embodiments, an average number of ISPs per T-reg cell in the Treg population is equal or less than about 4000. In some embodiments that may be combined with any of the preceding embodiments, an average number of ISPs per T-reg cell in the Treg population is from approximately 1500 to approximately 2500.

Other aspects of the present disclosure relate to a multi-component pharmaceutical treatment to be administered to a human subject in need thereof, the multi-component treatment comprising: (a) a myeloablative conditioning regimen comprising one or more doses of busulfan, fludarabine and thiotepa; (b) a solution comprising a first population of CD45⁺ cells comprising hematopoietic stem and progenitor cells (HSPCs); (c) a solution comprising a population of cells enriched for regulatory T cells (Tregs); (d) a solution comprising a third population of CD45⁺ cells wherein the third population of CD45⁺ cells comprise CD3⁺ conventional T cells (Tcons); and (e) a solution comprising one or more doses of a graft vs host disease (GVHD) prophylactic agent, wherein the conditioning regimen is formulated for administration from approximately two days to approximately ten days before any of (b) to (e).

In some embodiments that may be combined with any of the preceding embodiments, the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively. In some embodiments that may be combined with any of the preceding embodiments, the GVHD prophylactic agent is tacrolimus. In some embodiments that may be combined with any of the preceding embodiments, the one or more doses of the GVHD prophylactic agent comprise tacrolimus in an amount that maintains a target blood level of at least about 3 ng/ml for at least about 20 days after administration of the third population of CD45⁺ cells. In some embodiments that may be combined with any of the preceding embodiments, the HSPCs are CD34⁺. In some embodiments that may be combined with any of the preceding embodiments, the Tregs are CD4⁺ CD25⁺ CD127dim. In some embodiments that may be combined with any of the preceding embodiments, the population of cells enriched for Tregs comprises CD45⁺ cells, wherein more than about 90% of the CD45⁺ cells are Tregs. In some embodiments that may be combined with any of the preceding embodiments, the Tregs are CD4⁺ CD25⁺ CD127dimor CD4⁺ FOXP3⁺. In some embodiments that may be combined with any of the preceding embodiments, the first population of CD45⁺ cells comprising HSPCs comprises from approximately 5×10⁵ to approximately 2×10⁷ HSPCs per kilogram of actual or ideal body weight of said human subject. In some embodiments that may be combined with any of the preceding embodiments, the population of cells enriched for Tregs comprises from approximately 1×10⁵ to approximately 1×10⁷ Tregs per kilogram of actual or ideal body weight of said human subject. In some embodiments that may be combined with any of the preceding embodiments, the third population of CD45⁺ cells comprises from approximately 1×10⁵ to approximately 1×10⁷ Tcons per kilogram of actual or ideal body weight of said human subject. In some embodiments that may be combined with any of the preceding embodiments, the first population of CD45⁺ cells and the population of cells enriched for Tregs are formulated for administration prior to administration of the third population of CD45⁺ cells. In some embodiments that may be combined with any of the preceding embodiments, a first dose of the one or more doses of the GVHD prophylactic agent is formulated for administration subsequent to administration of the third population of CD45⁺ cells.

Other aspects of the present disclosure relate to a multi-component cellular therapy product comprising: a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, from approximately 1.5×10⁷ to approximately 1.5×10¹⁰ HSPCs, or from approximately 5.0×10⁵ to approximately 5.0×10⁸ HSPCs, wherein the first population of CD45⁺ cells is formulated with an excipient at a neutral pH; b) a second single dose transfer bag comprising a second population of isolated CD45⁺ cells comprising a dose of approximately 1.0×10⁵ to approximately 2.0×10⁷ fresh CD4⁺ CD25⁺ CD127^{dim} regulatory T cells (Tregs) per kilogram of body weight of the human subject receiving the product, from approximately 1.5×10⁷ to approximately 3.0×10⁹ fresh Tregs, or from approximately 5.0×10⁵ to approximately 1.0×10⁸ fresh Tregs, wherein the second population of isolated CD45⁺ cells is formulated with an excipient at a neutral pH; and c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons, wherein the third population of isolated CD45⁺ cells is formulated with an excipient at a neutral pH, wherein the excipient comprises one more cryoprotectants.

Other aspects of the present disclosure relate to a method of treating a human subject diagnosed with a hematologic malignancy, the method comprising administering to the human subject a multi-component pharmaceutical treatment comprising: a) a solution comprising a first population of isolated CD45⁺ cells comprising hematopoietic stem and progenitor cells (HSPCs); b) a solution comprising a second population of isolated CD45⁺ cells comprising regulatory T cells (Tregs); c) a solution comprising a third population of isolated CD45⁺ cells wherein the third population of isolated CD45⁺ cells comprise CD3⁺ conventional T cells (Tcons); and d) a solution comprising one or more doses of a graft vs host disease (GVHD) prophylactic agent, wherein the first population of isolated CD45⁺ cells, the population of cells enriched for Tregs, and the third population of isolated CD45⁺ cells are obtained from a single allogeneic donor that has at least one HLA mismatch relative to the human subject, and wherein incidence of non-relapse mortality of the human subject is decreased after administration and/or overall survival of the human subject is increased after administration, relative to a corresponding human subject that has been administered a standard myeloablative allogeneic hematopoietic stem cell transplant (alloHSCT) from a donor that has at least one HLA mismatch relative to the corresponding human subject. Other aspects of the present disclosure related to a multi-component pharmaceutical treatment of the present disclosure for use in treating a human subject diagnosed with a hematologic malignancy. Other aspects of the present disclosure relate to use of a multi-component pharmaceutical treatment of the present disclosure in the manufacture of a medicament for treating a human subject diagnosed with a hematologic malignancy.

Other aspects of the present disclosure relate to a multi-component pharmaceutical treatment to be administered to a human subject in need thereof, the multi-component treatment comprising: a) a solution comprising a first population of isolated CD45⁺ cells comprising hematopoietic stem and progenitor cells (HSPCs) and granulocytes wherein at most approximately 10% of the first population of isolated CD45⁺ cells comprise granulocytes; b) a solution comprising a second population of isolated CD45⁺ cells comprising regulatory T cells (Tregs); c) a solution comprising a third population of isolated CD45⁺ cells wherein the third population of isolated CD45⁺ cells comprise at least approximately 20% CD3⁺ conventional T cells (Tcons), at least approximately 10% monocytes, and at least approximately 10% granulocytes; and d) a solution comprising one or more doses of a graft vs host disease (GVHD) prophylactic agent, wherein the first population of isolated CD45⁺ cells, the population of cells enriched for Tregs, and the third population of isolated CD45⁺ cells have at least one HLA mismatch relative to the human subject, and wherein upon administration, the first population of isolated CD45⁺ cells, the population of cells enriched for Tregs, and the third population of isolated CD45⁺ cells decrease incidence of non-relapse mortality in the human subject and/or increase overall survival in human subject compared to incidence of non-relapse mortality and/or over survival in a corresponding human subject that has been administered a standard myeloablative allogeneic hematopoietic stem cell transplant (alloHSCT) that has at least one HLA mismatch relative to the corresponding human subject.

Other aspects of the present disclosure relate to a method of treating a human subject diagnosed with a hematologic malignancy, the method comprising administering to the human subject a multi-component pharmaceutical treatment comprising: a) a myeloablative conditioning regimen comprising one or more doses of busulfan, fludarabine and thiotepa; b) a solution comprising a first population of isolated CD45⁺ cells comprising hematopoietic stem and progenitor cells (HSPCs); c) a solution comprising a second population of isolated CD45⁺ cells comprising regulatory T cells (Tregs); d) a solution comprising a third population of isolated CD45⁺ cells wherein the third population of isolated CD45⁺ cells comprise CD3⁺ conventional T cells (Tcons); and e) a solution comprising one or more doses of a graft vs host disease (GVHD) prophylactic agent, wherein the conditioning regimen is administered from approximately two days to approximately ten days before any of (b) to (e). Other aspects of the present disclosure related to a multi-component pharmaceutical treatment of any of the preceding embodiments for use in treating a human subject diagnosed with a hematologic malignancy. Other aspects of the present disclosure relate to use of a multi-component pharmaceutical treatment of any of the preceding embodiments in the manufacture of a medicament for treating a human subject diagnosed with a hematologic malignancy.

Other aspects of the present disclosure relate to a method of treating a human subject diagnosed with a hematologic malignancy, the method comprising administering to the human subject a multi-component pharmaceutical treatment comprising: a) a solution comprising a first population of isolated CD45⁺ cells comprising hematopoietic stem and progenitor cells (HSPCs); b) a solution comprising a second population of isolated CD45⁺ cells comprising regulatory T cells (Tregs); c) a solution comprising a third population of isolated CD45⁺ cells wherein the third population of isolated CD45⁺ cells comprise CD3⁺ conventional T cells (Tcons); and d) a solution comprising one or more doses of a graft vs host disease (GVHD) prophylactic agent, wherein GVHD and relapse-free survival (GFRS) of the human subject increases by at least approximately 3.5-fold after administration, overall survival increased by at least approximately 1.25-fold after administration, and/or incidence of non-relapse mortality decreases by at least approximately 60% after administration, relative to a corresponding human subject that has been administered a standard myeloablative allogeneic hematopoietic stem cell transplant (alloHSCT). Other aspects of the present disclosure related to a multi-component pharmaceutical treatment of any of the preceding embodiments for use in treating a human subject diagnosed with a hematologic malignancy. Other aspects of the present disclosure relate to use of a multi-component pharmaceutical treatment of any of the preceding embodiments in the manufacture of a medicament for treating a human subject diagnosed with a hematologic malignancy.

Other aspects of the present disclosure relate to a multi-component pharmaceutical treatment to be administered to a human subject in need thereof, the multi-component treatment comprising: (a) a myeloablative conditioning regimen comprising one or more doses of busulfan, fludarabine and thiotepa; (b) a solution comprising a first population of isolated CD45⁺ cells comprising hematopoietic stem and progenitor cells (HSPCs); (c) a solution comprising a second population of isolated CD45⁺ cells comprising regulatory T cells (Tregs); (d) a solution comprising a third population of isolated CD45⁺ cells wherein the third population of isolated CD45⁺ cells comprise CD3⁺ conventional T cells (Tcons); and (e) a solution comprising one or more doses of a graft vs host disease (GVHD) prophylactic agent, wherein the conditioning regimen is formulated for administration from approximately two days to approximately ten days before any of (b) to (e).

Other aspects of the present disclosure relate to a method of preparing a multi-component cellular therapy product comprising: a) obtaining HSPC-mobilized peripheral blood apheresis from a donor that is allogeneic with respect to a human subject receiving the product; b) combining a first apheresis sample with one or more cryoprotectants and cryopreserving the first apheresis sample, wherein the first apheresis sample comprises from approximately 1.0×105 to approximately 4.0×107 conventional CD3⁺ T cells (Tcons) per kilogram of body weight of the human subject receiving the product, from approximately 5.0×105 to approximately 2.0×108 Tcons, or from approximately 1.5×107 to approximately 6.0×109 Tcons; c) sorting a second apheresis sample with one or more immune-separation particles (ISPs) specific for CD34 to obtain a CD34-enriched cell population and a CD34-depleted cell population, wherein the CD34-enriched cell population comprises from approximately 1.0×105 to approximately 1.0×108 CD34⁺ hematopoietic stem and progenitor cells (HSPCs) per kilogram of body weight of the human subject receiving the product, from approximately 5.0×105 to approximately 5.0×108 HSPCs, or from approximately 1.5×107 to approximately 1.5×1010 HSPCs; d) sorting the CD34-depleted cell population with one or more ISPs specific for CD25 to obtain a CD25-enriched cell population and a CD25-depleted cell population; and e) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×105 to approximately 2.0×107 CD4⁺ CD25⁺ CD127^{dim} regulatory T cells (Tregs) per kilogram of body weight of the human subject receiving the product, of from approximately 5.0×105 to approximately 1.0×108 isolated fresh Tregs, or from approximately 1.5×107 to approximately 3.0×109 isolated fresh Tregs, wherein the multi-component cellular therapy product comprises the Tcons, the HSPCs, and the Tregs.

Other aspects of the present disclosure relate to a method of preparing a multi-component cellular therapy product comprising: a) obtaining HSPC-mobilized peripheral blood apheresis from a donor that is allogeneic with respect to a human subject receiving the product; b) sorting a first apheresis sample with one or more immune-separation particles (ISPs) specific for CD3 to obtain a CD3-enriched cell population and a CD3-depleted cell population, wherein the CD3-enriched cell population comprises from approximately 1.0×105 to approximately 4.0×107 conventional CD3⁺ T cells (Tcons) per kilogram of body weight of the human subject receiving the product, from approximately 1.5×107 to approximately 6.0×109 Tcons, or from approximately 5.0×105 to approximately 2.0×108 Tcons; c) sorting a second apheresis sample with one or more ISPs specific for CD34 to obtain a CD34-enriched cell population and a CD34-depleted cell population, wherein the CD34-enriched cell population comprises from approximately 1.0×105 to approximately 1.0×108 CD34⁺ hematopoietic stem and progenitor cells (HSPCs) per kilogram of body weight of the human subject receiving the product, from approximately 1.5×107 to approximately 1.5×1010 HSPCs, or from approximately 5.0×105 to approximately 5.0×108 HSPCs; d) sorting the CD34-depleted cell population with one or more ISPs specific for CD25 to obtain a CD25-enriched cell population and a CD25-depleted cell population; and e) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×105 to approximately 2.0×107 CD4⁺ CD25⁺ CD127^{dim} regulatory T cells (Tregs) per kilogram of body weight of the human subject receiving the product, from approximately 1.5×107 to approximately 3.0×109 Tregs, or from approximately 5.0×105 to approximately 1.0×108 Tregs, wherein the multi-component cellular therapy product comprises the Tcons, the HSPCs, and the Tregs.

Other aspects of the present disclosure relate to a method of preparing a multi-component cellular therapy product comprising: a) obtaining HSPC-mobilized peripheral blood apheresis from a donor that is allogeneic with respect to a human subject receiving the product; b) cryopreserving from a first apheresis sample a cell population comprising from approximately 1.0×105 to approximately 4.0×107 conventional CD3⁺ T cells (Tcons) per kilogram of body weight of the human subject receiving the product, from approximately 1.5×107 to approximately 6.0×109 Tcons, or from approximately 5.0×105 to approximately 2.0×108 Tcons; c) sorting a second apheresis sample with one or more immune-separation particles (ISPs) specific for CD25 to obtain a CD25-enriched cell population and a CD25-depleted cell population, and sorting the CD25-depleted population with one or more ISPs specific for CD34 to obtain a CD34-enriched cell population and a CD34-depleted cell population, wherein the CD34-enriched cell population comprises from approximately 1.0×105 to approximately 1.0×108 CD34⁺ hematopoietic stem and progenitor cells (HSPCs) per kilogram of body weight of the human subject receiving the product, from approximately 1.5×107 to approximately 1.5×1010 HSPCs, or from approximately 5.0×105 to approximately 5.0×108 HSPCs; d) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×105 to approximately 2.0×107 CD4⁺ CD25⁺ CD127^{dim} regulatory T cells (Tregs) per kilogram of body weight of the human subject receiving the product, from approximately 1.5×107 to approximately 3.0×109 Tregs, or from approximately 5.0×105 to approximately 1.0×108 Tregs, wherein the multi-component cellular therapy product comprises the Tcons, the HSPCs, and the Tregs.

Other aspects of the present disclosure relate to a method of preparing a multi-component cellular therapy product comprising: a) obtaining HSPC-mobilized peripheral blood apheresis from a donor that is allogeneic with respect to a human subject receiving the product; b) combining a first apheresis sample with one or more cryoprotectants and cryopreserving the first apheresis sample, wherein the first apheresis sample comprises from approximately 1.0×105 to approximately 4.0×107 conventional CD3⁺ T cells (Tcons) per kilogram of body weight of the human subject receiving the product, from approximately 1.5×107 to approximately 6.0×109 Tcons, or from approximately 5.0×105 to approximately 2.0×108 Tcons; c) sorting a second apheresis sample with one or more immune-separation particles (ISPs) specific for CD25 to obtain a CD25-enriched cell population and a CD25-depleted cell population, sorting the CD25-depleted population with one or more ISPs specific for CD34 to approximately obtain a CD34-enriched cell population and a CD34-depleted cell population, wherein the CD34-enriched cell population comprises from approximately 1.0×105 to approximately 1.0×108 CD34⁺ hematopoietic stem and progenitor cells (HSPCs) per kilogram of body weight of the human subject receiving the product, from approximately 1.5×107 to approximately 1.5×1010 HSPCs, or from approximately 5.0×105 to approximately 5.0×108 HSPCs; d) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×105 to approximately 2.0×107 CD4⁺ CD25⁺ CD127^{dim} regulatory T cells (Tregs) per kilogram of body weight of the human subject receiving the product, from approximately 1.5×107 to approximately 3.0×109 Tregs, or from approximately 5.0×105 to approximately 1.0×108 Tregs, wherein the multi-component cellular therapy product comprises the Tcons, the HSPCs, and the Tregs.

Other aspects of the present disclosure relate to a method of preparing a multi-component cellular therapy product comprising: a) obtaining HSPC-mobilized peripheral blood apheresis from a donor that is allogeneic with respect to a human subject receiving the product; b) sorting a first apheresis sample with one or more immune-separation particles (ISPs) specific for CD3 to obtain a CD3-enriched cell population and a CD3-depleted cell population, wherein the CD3-enriched cell population comprises from approximately 1.0×105 to approximately 4.0×107 conventional CD3⁺ T cells (Tcons) per kilogram of body weight of the human subject receiving the product, from approximately 1.5×107 to approximately 6.0×109 Tcons, from approximately 5.0×105 to approximately 2.0×108 Tcons; c) sorting a second apheresis sample with one or more ISPs specific for CD25 to obtain a CD25-enriched cell population and a CD25-depleted cell population, sorting the CD25-depleted population with one or more ISPs specific for CD34 to obtain a CD34-enriched cell population and a CD34-depleted cell population, wherein the CD34-enriched cell population comprises from approximately 1.0×105 to approximately 1.0×108 CD34⁺ hematopoietic stem and progenitor cells (HSPCs) per kilogram of body weight of the human subject receiving the product, from approximately 1.5×107 to approximately 1.5×1010 HSPCs, or from approximately 5.0×105 to approximately 5.0×108 HSPCs; d) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×105 to approximately 2.0×107 CD4⁺ CD25⁺ CD127^{dim} regulatory T cells (Tregs) per kilogram of body weight of the human subject receiving the product, from approximately 1.5×107 to approximately 3.0×109 Tregs, or from approximately 5.0×105 to approximately 1.0×108 Tregs, wherein the multi-component cellular therapy product comprises the Tcons, the HSPCs, and the Tregs.

Other aspects of the present disclosure relate to a method of preparing a multi-component cellular therapy product comprising: a) obtaining HSPC-mobilized peripheral blood apheresis from a donor that is allogeneic with respect to a human subject receiving the product; b) cryopreserving a first apheresis sample, wherein the first apheresis sample comprises from approximately 1.0×105 to approximately 4.0×107 conventional CD3⁺ T cells (Tcons) per kilogram of body weight of the human subject receiving the product, from approximately 1.5×107 to approximately 6.0×109 Tcons, or from approximately 5.0×105 to approximately 2.0×108 Tcons; c) selecting from a second apheresis sample a CD34-enriched cell population comprising from approximately 1.0×105 to approximately 1.0×108 CD34⁺ hematopoietic stem and progenitor cells (HSPCs) per kilogram of body weight of the human subject receiving the product, from approximately 1.5×107 to approximately 1.5×1010 HSPCs, from approximately 5.0×105 to approximately 5.0×108 HSPCs; and d) selecting from the second apheresis sample a CD 25-enriched cell population that is CD4⁺ and CD127^{dim}, wherein the CD4⁺ and CD127dim cell population comprises from approximately 1.0×105 to approximately 2.0×107 CD4⁺ CD25+CD127^{dim} regulatory T cells (Tregs) per kilogram of body weight of the human subject receiving the product, from approximately 1.5×107 to approximately 3.0×109 Tregs, or from approximately 5.0×105 to approximately 1.0×108 Tregs, wherein the multi-component cellular therapy product comprises the Tcons, the HSPCs, and the Tregs.

Other aspects of the present disclosure relate to a method of preparing a multi-component cellular therapy product comprising: a) obtaining HSPC-mobilized peripheral blood apheresis sample from a donor that is allogeneic with respect to a human subject receiving the product; and b) selecting from the apheresis sample a first sample comprising from approximately 1.0×105 to approximately 1.0×108 CD34⁺ hematopoietic stem and progenitor cells (HSPCs) per kilogram of body weight of the human subject receiving the product, from approximately 1.5×107 to approximately 1.5×1010 HSPCs, from approximately 5.0×105 to approximately 5.0×108 HSPCs, a second sample comprising from approximately 1.0×105 to approximately 2.0×107 CD4⁺ CD25⁺ CD127^{dim} regulatory T cells (Tregs) per kilogram of body weight of the human subject receiving the product, from approximately 1.5×107 to approximately 3.0×109 Tregs, or from approximately 5.0×105 to approximately 1.0×108 Tregs, and a third sample comprising from approximately 1.0×105 to approximately 4.0×107 conventional CD3⁺ T cells (Tcons) per kilogram of body weight of the human subject receiving the product, from approximately 1.5×107 to approximately 6.0×109 Tcons, or from approximately 5.0×105 to approximately 2.0×108 Tcons, wherein the multi-component cellular therapy product comprises the Tcons, the HSPCs, and the Tregs.

Other aspects of the present disclosure relate to a multi-component cellular therapy product or a multi-component pharmaceutical treatment produced or prepared by a method of the any of the preceding embodiments.

Other aspects of the present disclosure relate to a method of treating a human subject having or suspected of having acute leukemia in complete remission, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having acute leukemia in complete remission. Other aspects of the present disclosure relate to use of a multi-component cellular therapy of the present disclosure in the manufacture of a medicament for treating a human subject having or suspected of having acute leukemia in complete remission.

Other aspects of the present disclosure relate to a method of treating a human subject having or suspected of having active leukemia, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25+CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having active leukemia. Other aspects of the present disclosure relate to use of a multi-component cellular therapy of the present disclosure in the manufacture of a medicament for treating a human subject having or suspected of having active leukemia.

Other aspects of the present disclosure relate to a method of treating a human subject having or suspected of having primary refractory acute leukemia or acute leukemia with minimal residual disease, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having primary refractory acute leukemia or acute leukemia with minimal residual disease. Other aspects of the present disclosure relate to use of a multi-component cellular therapy of the present disclosure in the manufacture of a medicament for treating a buman subject having or suspected of having primary refractory acute leukemia or acute leukemia with minimal residual disease.

Other aspects of the present disclosure relate to a method of treating a human subject having or suspected of having low-risk acute myeloid leukemia (AML), the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having low-risk acute myeloid leukemia (AML). Other aspects of the present disclosure relate to use of a multi-component cellular therapy of the present disclosure in the manufacture of a medicament for treating a human subject having or suspected of having low-risk acute myeloid leukemia (AML)

Other aspects of the present disclosure relate to a method of treating a human subject having or suspected of having high-risk acute myeloid leukemia (AML) in complete remission, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having high-risk acute myeloid leukemia (AML) in complete remission. Other aspects of the present disclosure relate to use of a multi-component cellular therapy of the present disclosure in the manufacture of a medicament for treating a human subject having or suspected of having high-risk acute myeloid leukemia (AML) in complete remission.

Other aspects of the present disclosure relate to a method of treating a human subject having or suspected of having chronic myelogenous leukemia (CML), the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having chronic myelogenous leukemia (CML). Other aspects of the present disclosure relate to use of a multi-component cellular therapy of the present disclosure in the manufacture of a medicament for treating a human subject having or suspected of having chronic myelogenous leukemia (CML).

Other aspects of the present disclosure relate to a method of treating a human subject having or suspected of having high-risk myelodysplastic syndrome, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having high-risk myelodysplastic syndrome. Other aspects of the present disclosure relate to use of a multi-component cellular therapy of the present disclosure in the manufacture of a medicament for treating a human subject having or suspected of having high-risk myelodysplastic syndrome.

Other aspects of the present disclosure relate to a method of treating a human subject having or suspected of having therapy-related myelodysplastic syndrome and/or secondary myelodysplastic syndrome, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127dim regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having therapy-related myelodysplastic syndrome and/or secondary myelodysplastic syndrome. Other aspects of the present disclosure relate to use of a multi-component cellular therapy of the present disclosure in the manufacture of a medicament for treating a human subject having or suspected of having therapy-related myelodysplastic syndrome and/or secondary myelodysplastic syndrome.

Other aspects of the present disclosure relate to a method of treating a human subject having or suspected of having a myeloproliferative syndrome, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having a myeloproliferative syndrome. Other aspects of the present disclosure relate to use of a multi-component cellular therapy of the present disclosure in the manufacture of a medicament for treating a human subject having or suspected of having a myeloproliferative syndrome.

Other aspects of the present disclosure relate to a method of treating a human subject having or suspected of having non-Hodgkin lymphoma with poor risk features not suitable for autologous hematopoietic cell transplant (HCT), the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having non-Hodgkin lymphoma with poor risk features not suitable for autologous hematopoietic cell transplant (HCT). Other aspects of the present disclosure relate to use of a multi-component cellular therapy of the present disclosure in the manufacture of a medicament for treating a human subject having or suspected of having non-Hodgkin lymphoma with poor risk features not suitable for autologous hematopoietic cell transplant (HCT).

Other aspects of the present disclosure relate to a method of treating a human subject having or suspected of having multiple sclerosis, the method comprising administering to the human subject a multi-component cellular therapy comprising: a) a solution comprising a first population of isolated CD45⁺ cells comprising CD34⁺ hematopoietic stem and progenitor cells (HSPCs); b) a solution comprising a second population of isolated CD45⁺ cells comprising fresh CD4⁺ CD25⁺ CD127^{dim} regulatory T cells (Tregs); and c) a solution comprising a third population of isolated CD45⁺ cells comprising conventional CD3⁺ T cells (Tcons). In some embodiments, the multi-component cellular therapy is a multi-component cellular therapy product of the present disclosure, or a multi-component pharmaceutical treatment of the present disclosure. Other aspects of the present disclosure related to a multi-component cellular therapy of the present disclosure for use in treating a human subject having or suspected of having multiple sclerosis. Other aspects of the present disclosure relate to use of a multi-component cellular therapy of the present disclosure in the manufacture of a medicament for treating a human subject having or suspected of having multiple sclerosis.

Other aspects of the present disclosure relate to a method of treating a human subject having or suspected of having a hematologic malignancy with a multi-component cellular therapy, the method comprising: a) administering a first population of isolated CD45⁺ cells comprising CD34⁺ hematopoietic stem and progenitor cells (HSPCs); b) following administration of the first population of isolated CD45⁺ cells, administering a second population of isolated CD45⁺ cells comprising isolated fresh CD4⁺ CD25⁺ CD127^{dim} regulatory T cells (Tregs); and c) administering a third population of isolated CD45⁺ cells comprising conventional CD3⁺ T cells (Tcons) from approximately 12 hours to approximately 120 hours after administration of the second population of isolated CD45⁺ cells, wherein the multi-component cellular therapy comprises the first population of isolated CD45⁺ cells, the second population of isolated CD45⁺ cells, and the third population of isolated CD45⁺ cells. Other aspects of the present disclosure related to a multi-component cellular therapy of the present disclosure for use in treating a human subject having or suspected of having a hematologic malignancy. Other aspects of the present disclosure relate to use of a multi-component cellular therapy of the present disclosure in the manufacture of a medicament for treating a human subject having or suspected of having a hematologic malignancy.

Other aspects of the present disclosure relate to a multi-component cellular therapy product comprising: a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, approximately 1.5×10⁷ to approximately 1.5×10¹⁰ HSPCs, or approximately 5.0×10⁵ to approximately 5.0×10⁸ HSPCs, wherein the first population of CD45⁺ cells is formulated with an excipient at a neutral pH; b) a second single dose transfer bag comprising a second population of isolated CD45⁺ cells comprising a dose of approximately 1.0×10⁵ to approximately 2.0×10⁷ fresh CD4⁺ CD25 CD127^{dim} regulatory T cells (Tregs) per kilogram of body weight of the human subject receiving the product, approximately 1.5×10⁷ to approximately 3.0×10⁹ isolated fresh Tregs, or approximately 5.0×10⁵ to approximately 1.0×10⁸ isolated fresh Tregs, wherein the second population of isolated CD45⁺ cells is formulated with an excipient at a neutral pH optionally wherein the first single dose transfer bag and the second single dose transfer bag are the same transfer bag; and c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, approximately 1.5×10⁷ to approximately 6.0×10⁹ Tcons, or approximately 5.0×10⁵ to approximately 2.0×10⁸ Tcons, wherein the third population of isolated CD45⁺ cells is formulated with an excipient at a neutral pH, wherein the excipient comprises one more cryoprotectants.

In some embodiments that may be combined with any of the preceding embodiments, a) the first population of isolated CD45⁺ cells comprises a dose of approximately 1.0×10⁵ or more HSPCs per kilogram of body weight of the human subject receiving the product, approximately 2.0×10⁵ or more HSPCs per kilogram of body weight of the human subject receiving the product, approximately 3.0×10⁵ or more HSPCs per kilogram of body weight of the human subject receiving the product, approximately 4.0×10⁵ or more HSPCs per kilogram of body weight of the human subject receiving the product, approximately 5.0×10⁵ or more HSPCs per kilogram of body weight of the human subject receiving the product, approximately 6.0×10⁵ or more HSPCs per kilogram of body weight of the human subject receiving the product, approximately 7.0×10⁵ or more HSPCs per kilogram of body weight of the human subject receiving the product, approximately 8.0×10⁵ or more HSPCs per kilogram of body weight of the human subject receiving the product, approximately 9.0×10⁵ or more HSPCs per kilogram of body weight of the human subject receiving the product, approximately 1.0×10⁶ or more HSPCs per kilogram of body weight of the human subject receiving the product, approximately 1.5×10⁶ or more HSPCs per kilogram of body weight of the human subject receiving the product, approximately 2.0×10⁶ or more HSPCs per kilogram of body weight of the human subject receiving the product, approximately 2.5×10⁶ or more HSPCs per kilogram of body weight of the human subject receiving the product, approximately 3.0×10⁶ or more HSPCs per kilogram of body weight of the human subject receiving the product, approximately 3.5×10⁶ or more HSPCs per kilogram of body weight of the human subject receiving the product, approximately 4.0×10⁶ or more HSPCs per kilogram of body weight of the human subject receiving the product, approximately 4.5×10⁶ or more HSPCs per kilogram of body weight of the human subject receiving the product, approximately 5.0×10⁶ or more HSPCs per kilogram of body weight of the human subject receiving the product, approximately 5.5×10⁶ or more HSPCs per kilogram of body weight of the human subject receiving the product, approximately 6.0×10⁶ or more HSPCs per kilogram of body weight of the human subject receiving the product, approximately 6.5×10⁶ or more HSPCs per kilogram of body weight of the human subject receiving the product, approximately 7.0×10⁶ or more HSPCs per kilogram of body weight of the human subject receiving the product, approximately 7.5×10⁶ or more HSPCs per kilogram of body weight of the human subject receiving the product, approximately 8.0×10⁶ or more HSPCs per kilogram of body weight of the human subject receiving the product, approximately 8.5×10⁶ or more HSPCs per kilogram of body weight of the human subject receiving the product, approximately 9.0×10⁶ or more HSPCs per kilogram of body weight of the human subject receiving the product, approximately 9.5×10⁶ or more HSPCs per kilogram of body weight of the human subject receiving the product, approximately 1.0×10⁷ or more HSPCs per kilogram of body weight of the human subject receiving the product, approximately 1.5×10⁷ or more HSPCs per kilogram of body weight of the human subject receiving the product, approximately 2.0×10⁷ or more HSPCs per kilogram of body weight of the human subject receiving the product, approximately 2.5×10⁷ or more HSPCs per kilogram of body weight of the human subject receiving the product, approximately 3.0×10⁷ or more HSPCs per kilogram of body weight of the human subject receiving the product, approximately 3.5×10⁷ or more HSPCs per kilogram of body weight of the human subject receiving the product, approximately 4.0×10⁷ or more HSPCs per kilogram of body weight of the human subject receiving the product, approximately 4.5×10⁷ or more HSPCs per kilogram of body weight of the human subject receiving the product, approximately 5.0×10⁷ or more HSPCs per kilogram of body weight of the human subject receiving the product, approximately 5.5×10⁷ or more HSPCs per kilogram of body weight of the human subject receiving the product, approximately 6.0×10⁷ or more HSPCs per kilogram of body weight of the human subject receiving the product, approximately 6.5×10⁷ or more HSPCs per kilogram of body weight of the human subject receiving the product, approximately 7.0×10⁷ or more HSPCs per kilogram of body weight of the human subject receiving the product, approximately 7.5×10⁷ or more HSPCs per kilogram of body weight of the human subject receiving the product, approximately 8.0×10⁷ or more HSPCs per kilogram of body weight of the human subject receiving the product, approximately 8.5×10⁷ or more HSPCs per kilogram of body weight of the human subject receiving the product, approximately 9.0×10⁷ or more HSPCs per kilogram of body weight of the human subject receiving the product, approximately 9.5×10⁷ or more HSPCs per kilogram of body weight of the human subject receiving the product, or approximately 1.0×10⁸ or more HSPCs per kilogram of body weight of the human subject receiving the product; and/or b) the first population of isolated CD45⁺ cells comprises a dose of approximately 5.0×10⁵ or more HSPCs, approximately 6.0×10⁵ or more HSPCs, approximately 7.0×10⁵ or more HSPCs, approximately 8.0×10⁵ or more HSPCs, approximately 9.0×10⁵ or more HSPCs, approximately 1.0×10⁶ or more HSPCs, approximately 1.5×10⁶ or more HSPCs, approximately 2.0×10⁶ or more HSPCs, approximately 2.5×10⁶ or more HSPCs, approximately 3.0×10⁶ or more HSPCs, approximately 3.5×10⁶ or more HSPCs, approximately 4.0×10⁶ or more HSPCs, approximately 4.5×10⁶ or more HSPCs, approximately 5.0×10⁶ or more HSPCs, approximately 5.5×10⁶ or more HSPCs, approximately 6.0×10⁶ or more HSPCs, approximately 6.5×10⁶ or more HSPCs, approximately 7.0×10⁶ or more HSPCs, approximately 7.5×10⁶ or more HSPCs, approximately 8.0×10⁶ or more HSPCs, approximately 8.5×10⁶ or more HSPCs, approximately 9.0×10⁶ or more HSPCs, approximately 9.5×10⁶ or more HSPCs, approximately 1.0×10⁷ or more HSPCs, approximately 1.5×10⁷ or more HSPCs, approximately 2.0×10⁷ or more HSPCs, approximately 2.5×10⁷ or more HSPCs, approximately 3.0×10⁷ or more HSPCs, approximately 3.5×10⁷ or more HSPCs, approximately 4.0×10⁷ or more HSPCs, approximately 4.5×10⁷ or more HSPCs, approximately 5.0×10⁷ or more HSPCs, approximately 5.5×10⁷ or more HSPCs, approximately 6.0×10⁷ or more HSPCs, approximately 6.5×10⁷ or more HSPCs, approximately 7.0×10⁷ or more HSPCs, approximately 7.5×10⁷ or more HSPCs, approximately 8.0×10⁷ or more HSPCs, approximately 8.5×10⁷ or more HSPCs, approximately 9.0×10⁷ or more HSPCs, approximately 9.5×10⁷ or more HSPCs, approximately 1.0×10⁸ or more HSPCs, approximately 1.5×10⁸ or more HSPCs, approximately 2.0×10⁸ or more HSPCs, approximately 2.5×10⁸ or more HSPCs, approximately 3.0×10⁸ or more HSPCs, approximately 3.5×10⁸ or more HSPCs, approximately 4.0×10⁸ or more HSPCs, approximately 4.5×10⁸ or more HSPCs, approximately 5.0×10⁸ or more HSPCs, approximately 5.5×10⁸ or more HSPCs, approximately 6.0×10⁸ or more HSPCs, approximately 6.5×10⁸ or more HSPCs, approximately 7.0×10⁸ or more HSPCs, approximately 7.5×10⁸ or more HSPCs, approximately 8.0×10⁸ or more HSPCs, approximately 8.5×10⁸ or more HSPCs, approximately 9.0×10⁸ or more HSPCs, approximately 9.5×10⁸ or more HSPCs, approximately 1.0×10⁹ or more HSPCs, approximately 1.5×10⁹ or more HSPCs, approximately 2.0×10⁹ or more HSPCs, approximately 2.5×10⁹ or more HSPCs, approximately 3.0×10⁹ or more HSPCs, approximately 3.5×10⁹ or more HSPCs, approximately 4.0×10⁹ or more HSPCs, approximately 4.5×10⁹ or more HSPCs, approximately 5.0×10⁹ or more HSPCs, approximately 5.5×10⁹ or more HSPCs, approximately 6.0×10⁹ or more HSPCs, approximately 6.5×10⁹ or more HSPCs, approximately 7.0×10⁹ or more HSPCs, approximately 7.5×10⁹ or more HSPCs, approximately 8.0×10⁹ or more HSPCs, approximately 8.5×10⁹ or more HSPCs, approximately 9.0×10⁹ or more HSPCs, approximately 9.5×10⁹ or more HSPCs, approximately 1.0×10¹⁰ or more HSPCs, or approximately 1.5×10¹⁰ or more HSPCs; and/or c) the second population of isolated CD45⁺ cells comprises a dose of approximately 1.0×10⁵ or more isolated fresh Tregs per kilogram of body weight of the human subject receiving the product, approximately 2.0×10⁵ or more isolated fresh Tregs per kilogram of body weight of the human subject receiving the product, approximately 3.0×10⁵ or more isolated fresh Tregs per kilogram of body weight of the human subject receiving the product, approximately 4.0×10⁵ or more isolated fresh Tregs per kilogram of body weight of the human subject receiving the product, approximately 5.0×10⁵ or more isolated fresh Tregs per kilogram of body weight of the human subject receiving the product, approximately 6.0×10⁵ or more isolated fresh Tregs per kilogram of body weight of the human subject receiving the product, approximately 7.0×10⁵ or more isolated fresh Tregs per kilogram of body weight of the human subject receiving the product, approximately 8.0×10⁵ or more isolated fresh Tregs per kilogram of body weight of the human subject receiving the product, approximately 9.0×10⁵ or more isolated fresh Tregs per kilogram of body weight of the human subject receiving the product, approximately 1.0×10⁶ or more isolated fresh Tregs per kilogram of body weight of the human subject receiving the product, approximately 1.5×10⁶ or more isolated fresh Tregs per kilogram of body weight of the human subject receiving the product, approximately 2.0×10⁶ or more isolated fresh Tregs per kilogram of body weight of the human subject receiving the product, approximately 2.5×10⁶ or more isolated fresh Tregs per kilogram of body weight of the human subject receiving the product, approximately 3.0×10⁶ or more isolated fresh Tregs per kilogram of body weight of the human subject receiving the product, approximately 3.5×10⁶ or more isolated fresh Tregs per kilogram of body weight of the human subject receiving the product, approximately 4.0×10⁶ or more isolated fresh Tregs per kilogram of body weight of the human subject receiving the product, approximately 4.5×10⁶ or more isolated fresh Tregs per kilogram of body weight of the human subject receiving the product, approximately 5.0×10⁶ or more isolated fresh Tregs per kilogram of body weight of the human subject receiving the product, approximately 5.5×10⁶ or more isolated fresh Tregs per kilogram of body weight of the human subject receiving the product, approximately 6.0×10⁶ or more isolated fresh Tregs per kilogram of body weight of the human subject receiving the product, approximately 6.5×10⁶ or more isolated fresh Tregs per kilogram of body weight of the human subject receiving the product, approximately 7.0×10⁶ or more isolated fresh Tregs per kilogram of body weight of the human subject receiving the product, approximately 7.5×10⁶ or more isolated fresh Tregs per kilogram of body weight of the human subject receiving the product, approximately 8.0×10⁶ or more isolated fresh Tregs per kilogram of body weight of the human subject receiving the product, approximately 8.5×10⁶ or more isolated fresh Tregs per kilogram of body weight of the human subject receiving the product, approximately 9.0×10⁶ or more isolated fresh Tregs per kilogram of body weight of the human subject receiving the product, approximately 9.5×10⁶ or more isolated fresh CD4⁺ CD25⁺ CD127^{dim} Tregs per kilogram of body weight of the human subject receiving the product, approximately 1.0×10⁷ or more isolated fresh Tregs per kilogram of body weight of the human subject receiving the product, approximately 1.5×10⁷ or more isolated fresh Tregs per kilogram of body weight of the human subject receiving the product, or approximately 2.0×10⁷ or more isolated fresh Tregs per kilogram of body weight of the human subject receiving the product, optionally wherein the Tregs are FOXP3⁺; and/or d) the second population of isolated CD45⁺ cells comprises a dose of approximately 5.0×10⁵ or more isolated fresh Tregs, approximately 6.0×10⁵ or more isolated fresh Tregs, approximately 7.0×10⁵ or more isolated fresh Tregs, approximately 8.0×10⁵ or more isolated fresh Tregs, approximately 9.0×10⁵ or more isolated fresh Tregs, approximately 1.0×10⁶ or more isolated fresh Tregs, approximately 1.5×106 or more isolated fresh Tregs, approximately 2.0×10⁶ or more isolated fresh Tregs, approximately 2.5×10⁶ or more isolated fresh Tregs, approximately 3.0×10⁶ or more isolated fresh Tregs, approximately 3.5×10⁶ or more isolated fresh Tregs, approximately 4.0×10⁶ or more isolated fresh Tregs, approximately 4.5×10⁶ or more isolated fresh Tregs, approximately 5.0×10⁶ or more isolated fresh Tregs, approximately 5.5×10⁶ or more isolated fresh Tregs, approximately 6.0×10⁶ or more isolated fresh Tregs, approximately 6.5×10⁶ or more isolated fresh Tregs, approximately 7.0×10⁶ or more isolated fresh Tregs, approximately 7.5×10⁶ or more isolated fresh Tregs, approximately 8.0×10⁶ or more isolated fresh Tregs, approximately 8.5×106 or more isolated fresh Tregs, approximately 9.0×10⁶ or more isolated fresh Tregs, approximately 9.5×10⁶ or more isolated fresh Tregs, approximately 1.0×10⁷ or more isolated fresh Tregs, approximately 1.5×10⁷ or more isolated fresh Tregs, approximately 2.0×10⁷ or more isolated fresh Tregs, approximately 2.5×10⁷ or more isolated fresh Tregs, approximately 3.0×107 or more isolated fresh Tregs, approximately 3.5×10⁷ or more isolated fresh Tregs, approximately 4.0×10⁷ or more isolated fresh Tregs, approximately 4.5×10⁷ or more isolated fresh Tregs, approximately 5.0×10⁷ or more isolated fresh Tregs, approximately 5.5×10⁷ or more isolated fresh Tregs, approximately 6.0×10⁷ or more isolated fresh Tregs, approximately 6.5×107 or more isolated fresh Tregs, approximately 7.0×10⁷ or more isolated fresh Tregs, approximately 7.5×10⁷ or more isolated fresh Tregs, approximately 8.0×10⁷ or more isolated fresh Tregs, approximately 8.5×10⁷ or more isolated fresh Tregs, approximately 9.0×10⁷ or more isolated fresh Tregs, approximately 9.5×10⁷ or more isolated fresh Tregs, approximately 1.0×10⁸ or more isolated fresh Tregs, approximately 1.5×10⁸ or more isolated fresh Tregs, approximately 2.0×10⁸ or more isolated fresh Tregs, approximately 2.5×10⁸ or more isolated fresh Tregs, approximately 3.0×10⁸ or more isolated fresh Tregs, approximately 3.5×10⁸ or more isolated fresh Tregs, approximately 4.0×10⁸ or more isolated fresh Tregs, approximately 4.5×10⁸ or more isolated fresh Tregs, approximately 5.0×10⁸ or more isolated fresh Tregs, approximately 5.5×10⁸ or more isolated fresh Tregs, approximately 6.0×10⁸ or more isolated fresh Tregs, approximately 6.5×10⁸ or more isolated fresh Tregs, approximately 7.0×10⁸ or more isolated fresh Tregs, approximately 7.5×10⁸ or more isolated fresh Tregs, approximately 8.0×10⁸ or more isolated fresh Tregs, approximately 8.5×10⁸ or more isolated fresh Tregs, approximately 9.0×10⁸ or more isolated fresh Tregs, approximately 9.5×10⁸ or more isolated fresh Tregs, approximately 1.0×10⁹ or more isolated fresh Tregs, approximately 1.5×10⁹ or more isolated fresh Tregs, approximately 2.0×10⁹ or more isolated fresh Tregs, approximately 2.5×109 or more isolated fresh Tregs, or approximately 3.0×10⁹ or more isolated fresh Tregs, optionally wherein the Tregs are FOXP3⁺; and/or e) the third population of isolated CD45⁺ cells comprises a dose of approximately 1.0×10⁵ or more Tcons per kilogram of body weight of the human subject receiving the product, approximately 2.0×10⁵ or more Tcons per kilogram of body weight of the human subject receiving the product, approximately 3.0×10⁵ or more Tcons per kilogram of body weight of the human subject receiving the product, approximately 4.0×10⁵ or more Tcons per kilogram of body weight of the human subject receiving the product, approximately 5.0×10⁵ or more Tcons per kilogram of body weight of the human subject receiving the product, approximately 6.0×10⁵ or more Tcons per kilogram of body weight of the human subject receiving the product, approximately 7.0×10⁵ or more Tcons per kilogram of body weight of the human subject receiving the product, approximately 8.0×10⁵ or more Tcons per kilogram of body weight of the human subject receiving the product, approximately 9.0×10⁵ or more Tcons per kilogram of body weight of the human subject receiving the product, approximately 1.0×10⁶ or more Tcons per kilogram of body weight of the human subject receiving the product, approximately 1.5×10⁶ or more Tcons per kilogram of body weight of the human subject receiving the product, approximately 2.0×10⁶ or more Tcons per kilogram of body weight of the human subject receiving the product, approximately 2.5×10⁶ or more Tcons per kilogram of body weight of the human subject receiving the product, approximately 3.0×10⁶ or more Tcons per kilogram of body weight of the human subject receiving the product, approximately 3.5×10⁶ or more Tcons per kilogram of body weight of the human subject receiving the product, approximately 4.0×10⁶ or more Tcons per kilogram of body weight of the human subject receiving the product, approximately 4.5×10⁶ or more Tcons per kilogram of body weight of the human subject receiving the product, approximately 5.0×10⁶ or more Tcons per kilogram of body weight of the human subject receiving the product, approximately 5.5×10⁶ or more Tcons per kilogram of body weight of the human subject receiving the product, approximately 6.0×10⁶ or more Tcons per kilogram of body weight of the human subject receiving the product, approximately 6.5×10⁶ or more Tcons per kilogram of body weight of the human subject receiving the product, approximately 7.0×10⁶ or more Tcons per kilogram of body weight of the human subject receiving the product, approximately 7.5×10⁶ or more Tcons per kilogram of body weight of the human subject receiving the product, approximately 8.0×10⁶ or more Tcons per kilogram of body weight of the human subject receiving the product, approximately 8.5×10⁶ or more Tcons per kilogram of body weight of the human subject receiving the product, approximately 9.0×10⁶ or Tcons per kilogram of body weight of the human subject receiving the product, approximately 9.5×10⁶ or more Tcons per kilogram of body weight of the human subject receiving the product, approximately 1.0×10⁷ or more Tcons per kilogram of body weight of the human subject receiving the product, approximately 1.5×10⁷ or more Tcons per kilogram of body weight of the human subject receiving the product, approximately 2.0×10⁷ or more Tcons per kilogram of body weight of the human subject receiving the product, approximately 2.5×10⁷ or more Tcons per kilogram of body weight of the human subject receiving the product, approximately 3.0×10⁷ or more Tcons per kilogram of body weight of the human subject receiving the product, approximately 3.5×10⁷ or more Tcons per kilogram of body weight of the human subject receiving the product, or approximately 4.0×10⁷ or more Tcons per kilogram of body weight of the human subject receiving the product; and/or f) the third population of isolated CD45⁺ cells comprises a dose of approximately 5.0×10⁵ or more Tcons, approximately 6.0×10⁵ or more Tcons, approximately 7.0×10⁵ or more Tcons, approximately 8.0×10⁵ or more Tcons, approximately 9.0×10⁵ or more Tcons, approximately 1.0×10⁶ or more Tcons, approximately 1.5×10⁶ or more Tcons, approximately 2.0×10⁶ or more Tcons, approximately 2.5×10⁶ or more Tcons, approximately 3.0×10⁶ or more Tcons, approximately 3.5×10⁶ or more Tcons, approximately 4.0×10⁶ or more Tcons, approximately 4.5×10⁶ or more Tcons, approximately 5.0×10⁶ or more Tcons, approximately 5.5×10⁶ or more Tcons, approximately 6.0×10⁶ or more Tcons, approximately 6.5×10⁶ or more Tcons, approximately 7.0×10⁶ or more Tcons, approximately 7.5×10⁶ or more Tcons, approximately 8.0×10⁶ or more Tcons, approximately 8.5×10⁶ or more Tcons, approximately 9.0×10⁶ or Tcons, approximately 9.5×10⁶ or more Tcons, approximately 1.0×10⁷ or more Tcons, approximately 1.5×10⁷ or more Tcons, approximately 2.0×10⁷ or more Tcons, approximately 2.5×10⁷ or more Tcons, approximately 3.0×10⁷ or more Tcons, approximately 3.5×10⁷ or more Tcons, approximately 4.0×10⁷ or more Tcons, approximately 4.5×10⁷ or more Tcons, approximately 5.0×10⁷ or more Tcons, approximately 5.5×10⁷ or more Tcons, approximately 6.0×10⁷ or more Tcons, approximately 6.5×10⁷ or more Tcons, approximately 7.0×10⁷ or more Tcons, approximately 7.5×10⁷ or more Tcons, approximately 8.0×10⁷ or more Tcons, approximately 8.5×10⁷ or more Tcons, approximately 9.0×10⁷ or more Tcons, approximately 9.5×10⁷ or more Tcons, approximately 1.0×10⁸ or more Tcons, approximately 1.5×10⁸ or more Tcons, approximately 2.0×10⁸ or more Tcons, approximately 2.5×10⁸ or more Tcons, approximately 3.0×10⁸ or more Tcons, approximately 3.5×10⁸ or more Tcons, approximately 4.0×10⁸ or more Tcons, approximately 4.5×10⁸ or more Tcons, approximately 5.0×10⁸ or more Tcons, approximately 5.5×10⁸ or more Tcons, approximately 6.0×10⁸ or more Tcons, approximately 6.5×10⁸ or more Tcons, approximately 7.0×10⁸ or more Tcons, approximately 7.5×10⁸ or more Tcons, approximately 8.0×10⁸ or more Tcons, approximately 8.5×10⁸ or more Tcons, approximately 9.0×10⁸ or more Tcons, approximately 9.5×10⁸ or more Tcons, approximately 1.0×10⁹ or more Tcons, approximately 1.5×10⁹ or more Tcons, approximately 2.0×10⁹ or more Tcons, approximately 2.5×10⁹ or more Tcons, approximately 3.0×10⁹ or more Tcons, approximately 3.5×10⁹ or more Tcons, approximately 4.0×10⁹ or more Tcons, approximately 4.5×10⁹ or more Tcons, approximately 5.0×10⁹ or more Tcons, approximately 5.5×10⁹ or more Tcons, or approximately 6.0×10⁹ or more Tcons.

In some embodiments that may be combined with any of the preceding embodiments, the product further comprises a pharmaceutical composition comprising a dose of graft vs host disease (GVHD) prophylactic agent tacrolimus sufficient to maintain a trough blood level of approximately 5 ng/mL to approximately 10 ng/ml in the human subject receiving the product, optionally wherein the pharmaceutical composition comprises tacrolimus at a dose that ranges from approximately 0.01 mg per kilogram of body weight of the human subject receiving the product to approximately 0.50 mg per kilogram of body weight of the human subject receiving the product twice per day, optionally wherein the tacrolimus is dosed in an amount to maintain or that maintains a target blood level of approximately 1 ng/mL to approximately 10 ng/ml in the human subject receiving the product for approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the third population of CD45⁺ cells.

In some embodiments that may be combined with any of the preceding embodiments, a) the first population of isolated CD45⁺ cells, the second population of isolated CD45⁺ cells, and/or the third population of isolated CD45⁺ cells is formulated at a volume that ranges from approximately 5 mL to approximately 1 L; and/or b) the neutral pH ranges from approximately 6.8 to approximately 7.6; and/or c) the excipient comprises a transport buffer, optionally wherein the transport buffer comprises approximately 120 to approximately 160 mEq sodium and/or the transport buffer comprises approximately 270 to approximately 320 mOsmol/L total, optionally wherein the transport buffer is selected from: phosphate-buffered saline (PBS), human serum, PlasmaLyte, and any combination thereof, optionally wherein the transport buffer further comprises approximately 0.1% weight by volume to approximately 10% weight by volume of a human carrier protein, optionally wherein the human carrier protein is selected from: human serum albumin (HSA), intravenous immune globulin (IVIG), AB serum, and any combination thereof; and/or d) any of the first single dose bag, the second single dose bad, and the third single dose bag is a polyvinyl chloride (PVC) transfer bag or an ethylene vinyl acetate (EVA) transfer bag; and/or e) the one or more cryoprotectants are selected from: sorbitol, dimethyl sulfoxide (DMSO), propylene glycol, glycerol, polyvinylpyrrolidone (PVP), and polyethylene glycol (PEG), serum, HSA, hetastarch, CRYOSTOR CS2, CRYOSTOR CS5, and CRYOSTOR CS10.

In some embodiments that may be combined with any of the preceding embodiments, the first population of isolated CD45⁺ cells, the second population of isolated CD45⁺ cells, and the third population of isolated CD45⁺ cells are from an allogeneic donor having at least one HLA mismatch relative to the human subject receiving the product, optionally wherein the HLA-mismatched donor is unrelated to the human subject receiving the product or the HLA-mismatched donor is related to the human subject receiving the product, optionally wherein the at least one HLA mismatch is at an allele selected from: HLA-A, HLA-B, HLA-C, HLA-DRB1, and any combination thereof, optionally wherein the cells having at least one HLA mismatch are from a donor that is 6/8 HLA-mismatched relative to the human subject receiving the product or is 7/8 HLA-mismatched relative to the human subject receiving the product, optionally wherein the donor that has the at least one HLA mismatch relative to the human subject receiving the product has a mismatched HLA allele as a result of the donor being homozygous for the HLA allele while the human subject receiving the product is heterogeneous for the HLA allele, or the donor that has at least one HLA mismatch relative to the human subject receiving the product has a mismatched HLA allele as a result of the donor being heterozygous for the HLA allele while the human subject receiving the product is homozygous for the HLA allele, or the donor that has at least one HLA mismatch relative to the human subject receiving the product has a mismatched HLA allele as a result of both the donor and the human subject receiving the product being heterozygous for the HLA allele, optionally wherein upon administration the first population of isolated CD45⁺ cells, the second population of isolated CD45⁺ cells, and the third population of isolated CD45+ cells decrease incidence of non-relapse mortality in the human subject receiving the product and/or increase overall survival in the human subject receiving the product compared to incidence of non-relapse mortality and/or over survival in a corresponding human subject that has been administered a standard myeloablative allogeneic hematopoietic stem cell transplant (alloHSCT) that is 7/8 HLA-mismatched relative to the corresponding human subject.

Other aspects of the present disclosure relate to a method of treating a human subject having or suspected of having a hematologic malignancy, the method comprising administering to the human subject the multi-component cellular therapy product of any of the preceding embodiments, or a multi-component pharmaceutical treatment comprising: a) a solution comprising a first population of isolated CD45⁺ cells comprising hematopoietic stem and progenitor cells (HSPCs); b) a solution comprising a second population of isolated CD45⁺ cells comprising regulatory T cells (Tregs); c) a solution comprising a third population of isolated CD45⁺ cells wherein the third population of isolated CD45⁺ cells comprise CD3⁺ conventional T cells (Tcons); and d) a solution comprising one or more doses of a graft vs host disease (GVHD) prophylactic agent, wherein the first population of isolated CD45⁺ cells, the second population of isolated CD45⁺ cells, and the third population of isolated CD45⁺ cells are obtained from a single allogeneic donor that has at least one HLA mismatch relative to the human subject, and wherein incidence of non-relapse mortality of the human subject is decreased after administration and/or overall survival of the human subject is increased after administration, relative to a corresponding human subject that has been administered a standard myeloablative allogeneic hematopoietic stem cell transplant (alloHSCT) from a donor that has at least one HLA mismatch relative to the corresponding human subject, optionally wherein the hematologic malignancy is selected from: acute lymphocytic leukemia, acute myelogenous leukemia, chronic myelogenous leukemia, multiple myeloma, lymphoma, Hodgkin's lymphoma, non-Hodgkin lymphoma, myelodysplastic syndrome, myelofibrosis, and blastic plasmacytoid dendritic cell neoplasm (BPDCN), optionally wherein the administering comprises infusing into the human subject the first population of isolated CD45⁺ cells, the second population of isolated CD45⁺ cells, and the third population of isolated CD45⁺ cells, optionally wherein the third population of isolated CD45⁺ cells is administered from approximately 24 to approximately 120 hours after the first population of isolated CD45⁺ cells, optionally the third population of isolated CD45⁺ cells is administered from approximately 24 to approximately 120 hours after the second population of isolated CD45⁺ cells, optionally wherein the human subject does not develop higher than stage 2 GVHD within approximately 100 days, within approximately 180 days, within approximately 200 days, or within approximately 1 year of the administering of the third population of isolated CD45⁺ cells, optionally wherein the human subject has previously been or is concurrently being treated for the hematologic malignancy, optionally wherein the GVHD prophylactic agent is tacrolimus and is initially administered to the human subject at approximately 0.03 mg/kg human subject's actual or ideal body weight/day, optionally wherein the GVHD prophylactic agent is initially administered from approximately 12 hours to approximately 24 hours after the administering of the third population of isolated CD45⁺ cells, optionally wherein the tacrolimus is administered in an amount to maintain a target blood level of at least approximately 3 ng/ml for at least approximately 20 days or approximately 40 days after administering the third population of isolated CD45⁺ cells, optionally wherein the tacrolimus is administered for at least approximately 60 days, at least approximately 90 days, at least approximately 120 days, or at most approximately 160 days after administering the third population of isolated CD45⁺ cells, optionally wherein administration of the GVHD prophylactic agent is tapered starting at approximately 90 days after initial administration of the GVHD prophylactic agent, optionally wherein the method further comprises administering approximately 1000 mg of mycophenolate mofetil (MMF), optionally wherein the MMF is initially administered from approximately 12 hours to approximately 24 hours after the administering of the third population of isolated CD45⁺ cells, optionally wherein administration of the MMF is tapered starting at approximately 30 days, at approximately 35 days, at approximately 40 days, at approximately 41 days, at approximately 42 days, at approximately 43 days, at approximately 44 days, at approximately 45 days, at approximately 46 days, at approximately 47 days, at approximately 48 days, at approximately 49 days, or at approximately 50 days after initial administration of the MMF, optionally wherein the at least one HLA mismatch is at an allele selected from: HLA-A, HLA-B, HLA-C, HLA-DRB1, and any combination thereof, optionally wherein the allogeneic donor that has at least one HLA mismatch is 6/8 HLA-mismatched relative to the human subject or is 7/8 HLA-mismatched relative to the human subject, optionally wherein the allogeneic donor that has at least one HLA mismatch relative to the human subject has a mismatched HLA allele as a result of the allogeneic donor being homozygous for the HLA allele while the human subject is heterogeneous for the HLA allele, or the allogeneic donor that has at least one HLA mismatch relative to the human subject has a mismatched HLA allele as a result of the allogeneic donor being heterozygous for the HLA allele while the human subject is homozygous for the HLA allele, or the allogeneic donor that has at least one HLA mismatch relative to the human subject has a mismatched HLA allele as a result of both the allogeneic donor and the human subject being heterozygous for the HLA allele, optionally wherein the method further comprises a conditioning regimen, wherein the conditioning regimen is administered before any of (a) to (d), optionally wherein the conditioning regimen is a myeloablative conditioning regimen, optionally wherein the myeloablative conditioning regimen comprises at least three conditioning reagents, wherein at least one conditioning reagent comprises thiotepa, optionally wherein the myeloablative conditioning regimen comprises one or more doses of busulfan, fludarabine and thiotepa, optionally wherein the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively, optionally wherein GVHD and relapse-free survival (GFRS) of the human subject increases by at least approximately 3.5-fold after administration, overall survival increases by at least approximately 1.25-fold after administration, and/or incidence of non-relapse mortality decreases by at least approximately 60% after administration, relative to a corresponding human subject that has been administered a standard myeloablative allogeneic hematopoietic stem cell transplant (alloHSCT). Other aspects of the present disclosure related to a multi-component cellular therapy product of any of the preceding embodiments, or a multi-component pharmaceutical treatment of the present disclosure, for use in treating a human subject having or suspected of having a hematologic malignancy. Other aspects of the present disclosure relate to use of a multi-component cellular therapy product of any of the preceding embodiments, or a multi-component pharmaceutical treatment of the present disclosure, in the manufacture of a medicament for treating a human subject having or suspected of having a hematologic malignancy.

Other aspects of the present disclosure relates to a method of preparing a multi-component cellular therapy product comprising: a) obtaining an HSPC-mobilized peripheral blood apheresis sample from a donor that is allogeneic with respect to a human subject receiving the product; and b) selecting from the apheresis sample a first sample comprising from approximately 1.0×10⁵ to approximately 1.0×10⁸ CD34⁺ hematopoietic stem and progenitor cells (HSPCs) per kilogram of body weight of the human subject receiving the product, from approximately 1.5×10⁷ to approximately 1.5×10¹⁰ HSPCs, from approximately 5.0×10⁵ to approximately 5.0×10⁸ HSPCs, a second sample comprising from approximately 1.0×10⁵ to approximately 2.0×10⁷ CD4⁺ CD25⁺ CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, and a third sample comprising from approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons, wherein the multi-component cellular therapy product comprises the Tcons, the HSPCs, and the Tregs, optionally wherein the HSPCs are selected by sorting the first sample with one or more ISPs specific for CD34 and selecting a CD34-enriched population, optionally wherein the Tregs are selected by soring the second sample with one or more ISPs specific for CD25, one or more ISPs specific for CD4, and/or one or more ISPs specific for CD127, and selecting the CD4⁺ and CD127^{dim} cell population, optionally wherein the Tcons are selected by sorting the third sample with one or more ISPs specific for CD3, optionally wherein the third sample is cryopreserved. Other aspects of the present disclosure related to a multi-component cellular therapy product produced by the method of any of the preceding embodiments, optionally wherein the multi-component cellular therapy product is the multi-component cellular therapy product of any of the preceding embodiments.

Other aspects of the present disclosure relate to a method of treating a human subject having or suspected of having acute leukemia in complete remission, active leukemia, primary refractory acute leukemia, or acute leukemia with minimal residual disease, wherein the method comprises administering to the human subject the multi-component cellular therapy product of any of the preceding embodiments, optionally wherein the acute leukemia is in complete remission with incomplete hematologic recovery, optionally wherein minimal residual disease is present in the human subject or minimal residual disease is absent in the human subject, optionally wherein the acute leukemia is categorized as intermediate-risk to very high-risk acute leukemia, optionally wherein the acute leukemia is acute myeloid leukemia (AML), acute lymphoid leukemia (ALL), and/or mixed phenotype acute leukemia (MPAL).

Other aspects of the present disclosure relate to a method of treating a human subject having or suspected of having low-risk acute myeloid leukemia (AML), high-risk AML in complete remission, or chronic myelogenous leukemia (CML), wherein the method comprises administering to the human subject the multi-component cellular therapy product of any of the preceding embodiments, optionally wherein the high risk AML comprises a complex karyotype with 3 or more clonal chromosomal abnormalities, wherein the 3 or more clonal chromosomal abnormalities are each selected from: monosomal karyotype-5, 5q-, -7 or 7q-, t(11q23, t(9;11), inv(3), t(3,3)t(6;9)t(9;22), normal karyotype with a fms-like tyrosine kinase 3 (FLT3)-ITD mutation, and any combination thereof, optionally wherein the CML is in blast phase, is in second chronic phase, is in chronic phase, is accelerated, has a history of blast crisis, and/or is intolerant of one or more first-generation or second-generation tyrosine kinase inhibitors (TKIs). Other aspects of the present disclosure related to a multi-component cellular therapy product of any of the preceding embodiments for use in treating a human subject having or suspected of having low-risk acute myeloid leukemia (AML), high-risk AML in complete remission, or chronic myelogenous leukemia (CML). Other aspects of the present disclosure relate to use of a multi-component cellular therapy product of any of the preceding embodiments in the manufacture of a medicament for treating a human subject having or suspected of having low-risk acute myeloid leukemia (AML), high-risk AML in complete remission, or chronic myelogenous leukemia (CML).

Other aspects of the present disclosure relate to a method of treating a human subject having or suspected of having high-risk myelodysplastic syndrome, therapy-related myelodysplastic syndrome, and/or secondary myelodysplastic syndrome, wherein the method comprises administering to the human subject the multi-component cellular therapy product of any of the preceding embodiments, optionally wherein the human subject has active disease at time of treatment with the multi-component cellular therapy, optionally wherein the human subject has approximately 10% or less blast burden in their bone marrow, optionally, wherein the therapy-related myelodysplastic syndrome and/or secondary myelodysplastic syndrome is in complete remission and is categorized as intermediate-risk to high-risk. Other aspects of the present disclosure related to a multi-component cellular therapy product of any of the preceding embodiments for use in treating a human subject having or suspected of having high-risk myelodysplastic syndrome, therapy-related myelodysplastic syndrome, and/or secondary myelodysplastic syndrome. Other aspects of the present disclosure relate to use of a multi-component cellular therapy product of any of the preceding embodiments in the manufacture of a medicament for treating a human subject having or suspected of having high-risk myelodysplastic syndrome, therapy-related myelodysplastic syndrome, and/or secondary myelodysplastic syndrome.

Other aspects of the present disclosure relate to a method of treating a human subject having or suspected of having a myeloproliferative syndrome, the method comprising administering to the human subject the multi-component cellular therapy product of any of the preceding embodiments. Other aspects of the present disclosure related to a multi-component cellular therapy product of any of the preceding embodiments for use in treating a human subject having or suspected of having a myeloproliferative syndrome. Other aspects of the present disclosure relate to use of a multi-component cellular therapy product of any of the preceding embodiments in the manufacture of a medicament for treating a human subject having or suspected of having a myeloproliferative syndrome.

Other aspects of the present disclosure relate to a method of treating a human subject having or suspected of having non-Hodgkin lymphoma with poor risk features not suitable for autologous hematopoietic cell transplant (HCT), the method comprising administering to the human subject the multi-component cellular therapy product of any of the preceding embodiments. Other aspects of the present disclosure related to a multi-component cellular therapy product of any of the preceding embodiments for use in treating a human subject having or suspected of having non-Hodgkin lymphoma with poor risk features not suitable for autologous hematopoietic cell transplant (HCT). Other aspects of the present disclosure relate to use of a multi-component cellular therapy product of any of the preceding embodiments in the manufacture of a medicament for treating a human subject having or suspected of having non-Hodgkin lymphoma with poor risk features not suitable for autologous hematopoietic cell transplant (HCT).

In some embodiments that may be combined with any of the preceding embodiments, a) the third population of isolated CD45⁺ cells is administered from approximately 24 to approximately 120 hours after the first population of isolated CD45⁺ cells; and/or b) the third population of isolated CD45⁺ cells is administered from approximately 24 to approximately 120 hours after the second population of isolated CD45⁺ cells; and/or c) the multi-component cellular therapy further comprises a solution comprising one or more doses of a graft vs host disease (GVHD) prophylactic agent, optionally wherein the GVHS prophylactic agent is tacrolimus, optionally wherein the tacrolimus is provided in an amount sufficient to maintain a trough blood level of approximately 5 ng/ml to approximately 10 ng/ml in the human subject, optionally wherein the tacrolimus is provided at a dose that ranges from approximately 0.01 mg per kilogram of body weight of the human subject to 0.50 mg per kilogram of body weight of the human subject twice per day, optionally wherein the tacrolimus is initially administered from approximately 12 hours to approximately 24 hours after the third population of isolated CD45⁺ cells, optionally wherein administration of the tacrolimus is tapered starting at approximately 90 days after initial administration of the tacrolimus; and/or d) the first population of isolated CD45⁺ cells comprises from approximately 1.0×10⁵ to approximately 1.0×10⁸ HSPCs per kilogram of body weight of the human subject, from approximately 5.0×10⁵ to approximately 5.0×10⁸ HSPCs, or from approximately 1.5×10⁷ to approximately 1.5×10¹⁰ HSPCs; and/or e) the second population of isolated CD45⁺ cells comprises from approximately 1.0×10⁵ to approximately 2.0×10⁷ Tregs per kilogram of body weight of the human subject, from approximately 1.5×10⁷ to approximately 3.0×10⁹ Tregs, or from approximately 5.0×10⁵ to approximately 1.0×10⁸ Tregs, optionally wherein the Tregs are FOXP3⁺; and/or f) the third population of isolated CD45⁺ cells comprises from approximately 1.0×10⁵ to approximately 4.0×10⁷ Tcons per kilogram of body weight of the human subject, from approximately 5.0×10⁵ to approximately 2.0×10⁸ Tcons, or from approximately 1.5×10⁷ to approximately 6.0×10⁹ Tcons; and/or g) the human subject does not develop higher than stage 2 GVHD within approximately 100 days, within approximately 180 days, within approximately 200 days, or within 1 year of the administering of the third population of isolated CD45⁺ cell; and/or h) the first population of isolated CD45⁺ cells, the second population of isolated CD45⁺ cells, and the third population of isolated CD45⁺ cells are from an allogeneic donor having at least one HLA mismatch relative to the human subject, optionally wherein the HLA-mismatched donor is unrelated to the human subject or the HLA-mismatched donor is related to the human subject, optionally wherein the at least one HLA mismatch is at an allele selected from: HLA-A, HLA-B, HLA-C, HLA-DRB1, and any combination thereof, optionally wherein the donor that has at least one HLA mismatch is 6/8 HLA-mismatched relative to the human subject or is 7/8 HLA-mismatched relative to the human subject, optionally wherein the donor that has at least one HLA mismatch relative to the human subject has a mismatched HLA allele as a result of the donor being homozygous for the HLA allele while the human subject is heterogeneous for the HLA allele, or the donor that has at least one HLA mismatch relative to the human subject has a mismatched HLA allele as a result of the donor being heterozygous for the HLA allele while the human subject is homozygous for the HLA allele, or the donor that has at least one HLA mismatch relative to the human subject has a mismatched HLA allele as a result of both the donor and the human subject being heterozygous for the HLA allele; and/or i) wherein incidence of non-relapse mortality of the human subject is decreased after administration of the multi-component cellular therapy, relative to a corresponding human subject that has been administered a standard myeloablative allogeneic hematopoietic stem cell transplant (alloHSCT) from a donor that has at least one HLA mismatch relative to the corresponding human subject; and/or j) overall survival of the human subject is increased after administration of the multi-component cellular therapy, relative to a corresponding human subject that has been administered a standard myeloablative allogeneic hematopoietic stem cell transplant (alloHSCT) from a donor that has at least one HLA mismatch relative to the corresponding human subject; and/or k) the method further comprises collecting one or more, or two or more mobilized peripheral blood donations from the donor, optionally wherein the peripheral blood donations are mobilized by granulocyte colony-stimulating factor (G-CSF), granulocyte macrophage colony-stimulating factor (GM-CSF), plerixafor, or any combination thereof, optionally wherein at least one of the mobilized peripheral blood donations is processed and sorted using one or more immune-separation particles (ISPs) to enrich CD34⁺ cells and Tregs; and/or 1) the method further comprises a conditioning regimen, wherein the conditioning regimen is administered before administration of the multi-component cellular therapy, optionally wherein the conditioning regimen is administered from approximately two days to approximately ten days before administration of the multi-component cellular therapy, optionally wherein the conditioning regimen is a total body irradiation-based (TBI-based) regimen, optionally wherein the TBI-based regimen further comprises one or more conditioning reagents, optionally wherein the one or more conditioning reagents are selected from: cyclophosphamide, etoposide, thiotepa, and any combination thereof, optionally wherein the conditioning regimen is a myeloablative conditioning regimen, optionally wherein the myeloablative conditioning regimen comprises one or more conditioning reagents, optionally wherein the one or more conditioning reagents are selected from: thiotepa, busulfan, melphalan, fludarabine, cyclophosphamide, anti-thymocyte globulin (ATG), and any combination thereof, or wherein the myeloablative conditioning regimen comprises one or more doses of busulfan, fludarabine, and thiotepa, optionally wherein the one or more doses of busulfan, fludarabine, and thiotepa comprise from approximately 5 to approximately 12 mg of thiotepa per kilogram of body weight of the human subject, from approximately 7 to approximately 11 mg of busulfan per kilogram of body weight of the human subject, and from approximately 100 to approximately 200 mg of fludarabine per meter² body surface area respectively; and/or m) GVHD and relapse-free survival (GFRS) of the human subject increases by at least approximately 3.5-fold after administration of the multi-component cellular therapy, relative to a corresponding human subject that has been administered a standard myeloablative allogeneic hematopoietic stem cell transplant (alloHSCT); and/or n) overall survival increases by at least approximately 1.25-fold after administration of the multi-component cellular therapy, relative to a corresponding human subject that has been administered a standard myeloablative allogeneic hematopoietic stem cell transplant (alloHSCT); and/or o) incidence of non-relapse mortality decreases by at least approximately 60% after administration of the multi-component cellular therapy, relative to a corresponding human subject that has been administered a standard myeloablative allogeneic hematopoietic stem cell transplant (alloHSCT); and/or p) chronic GVHD-free survival increases after administration of the multi-component cellular therapy, relative to a corresponding human subject that has been administered a standard myeloablative allogeneic hematopoietic stem cell transplant (alloHSCT), optionally wherein the human subject experiences chronic GVHD-free survival for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or q) incidence of primary graft failure or secondary graft failure decreases after administration of the multi-component cellular therapy, relative to a corresponding human subject that has been administered a standard myeloablative allogeneic hematopoietic stem cell transplant (alloHSCT), optionally wherein the human subject is free of primary graft failure or secondary graft failure for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or r) incidence, severity, timing, or any combination thereof of Grade I to Grade IV acute GVHD decreases after administration of the multi-component cellular therapy, relative to a corresponding human subject that has been administered a standard myeloablative allogeneic hematopoietic stem cell transplant (alloHSCT), optionally wherein the human subject is free of Grade I to Grade IV acute GVHD for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or s) incidence of steroid-refractory acute GVHD decreases after administration of the multi-component cellular therapy, relative to a corresponding human subject that has been administered a standard myeloablative allogeneic hematopoietic stem cell transplant (alloHSCT), optionally wherein the human subject is free of steroid-refractory acute GVHD for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or t) GVHD and relapse-free survival (GFRS) increases after administration of the multi-component cellular therapy, relative to a corresponding human subject that has been administered a standard myeloablative allogeneic hematopoietic stem cell transplant (alloHSCT), optionally wherein the human subject experiences GFRS for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or u) relapse-free survival increases after administration of the multi-component cellular therapy, relative to a corresponding human subject that has been administered a standard myeloablative allogeneic hematopoietic stem cell transplant (alloHSCT), optionally wherein the human subject experiences relapse-free survival for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or v) incidence, severity, timing, or any combination thereof of moderate to severe chronic GVHD decreases after administration of the multi-component cellular therapy, relative to a corresponding human subject that has been administered a standard myeloablative allogeneic hematopoietic stem cell transplant (alloHSCT), optionally wherein the human subject is free of severe chronic GVHD for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or w) incidence, severity, timing, or any combination thereof of non-relapse mortality decreases after administration of the multi-component cellular therapy, relative to a corresponding human subject that has been administered a standard myeloablative allogeneic hematopoietic stem cell transplant (alloHSCT), optionally wherein the human subject does not experience non-relapse mortality for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or x) Grade 3 or higher infections decreases after administration of the multi-component cellular therapy, relative to a corresponding human subject that has been administered a standard myeloablative allogeneic hematopoietic stem cell transplant (alloHSCT); optionally wherein the human subject is free of Grade 3 or higher infections for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or y) incidence, timing, or both incidence and timing of neutrophil engraftment increases after administration of the multi-component cellular therapy, relative to a corresponding human subject that has been administered a standard myeloablative allogeneic hematopoietic stem cell transplant (alloHSCT), optionally wherein neutrophil engraftment occurs approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 22 days or more, approximately 22 days or more, approximately 23 days or more, approximately 24 days or more, approximately 25 days or more, approximately 26 days or more, approximately 27 days or more, approximately 28 days or more, approximately 29 days or more, approximately 30 days or more, approximately 33 days or more, approximately 33 days or more, approximately 33 days or more, approximately 34 days or more, approximately 35 days or more, approximately 36 days or more, approximately 37 days or more, approximately 38 days or more, approximately 39 days or more, approximately 40 days or more, approximately 44 days or more, approximately 44 days or more, approximately 44 days or more, approximately 44 days or more, approximately 45 days or more, approximately 46 days or more, approximately 47 days or more, approximately 48 days or more, approximately 49 days or more, approximately 50 days or more, approximately 55 days or more, approximately 55 days or more, approximately 55 days or more, approximately 55 days or more, approximately 55 days or more, approximately 56 days or more, approximately 57 days or more, approximately 58 days or more, approximately 59 days or more, approximately 60 days or more, approximately 66 days or more, approximately 66 days or more, approximately 66 days or more, approximately 66 days or more, approximately 66 days or more, approximately 66 days or more, approximately 67 days or more, approximately 68 days or more, approximately 69 days or more, approximately 70 days or more, approximately 77 days or more, approximately 77 days or more, approximately 77 days or more, approximately 77 days or more, approximately 77 days or more, approximately 77 days or more, approximately 77 days or more, approximately 78 days or more, approximately 79 days or more, approximately 80 days or more, approximately 88 days or more, approximately 88 days or more, approximately 88 days or more, approximately 88 days or more, approximately 88 days or more, approximately 88 days or more, approximately 88 days or more, approximately 88 days or more, approximately 89 days or more, or approximately 90 days or more after administration of the multi-component cellular therapy; and/or z) incidence, timing, or both incidence and timing of platelet engraftment increases after administration of the multi-component cellular therapy, relative to a corresponding human subject that has been administered a standard myeloablative allogeneic hematopoietic stem cell transplant (alloHSCT), optionally wherein platelet engraftment occurs approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 22 days or more, approximately 22 days or more, approximately 23 days or more, approximately 24 days or more, approximately 25 days or more, approximately 26 days or more, approximately 27 days or more, approximately 28 days or more, approximately 29 days or more, approximately 30 days or more, approximately 33 days or more, approximately 33 days or more, approximately 33 days or more, approximately 34 days or more, approximately 35 days or more, approximately 36 days or more, approximately 37 days or more, approximately 38 days or more, approximately 39 days or more, approximately 40 days or more, approximately 44 days or more, approximately 44 days or more, approximately 44 days or more, approximately 44 days or more, approximately 45 days or more, approximately 46 days or more, approximately 47 days or more, approximately 48 days or more, approximately 49 days or more, approximately 50 days or more, approximately 55 days or more, approximately 55 days or more, approximately 55 days or more, approximately 55 days or more, approximately 55 days or more, approximately 56 days or more, approximately 57 days or more, approximately 58 days or more, approximately 59 days or more, approximately 60 days or more, approximately 66 days or more, approximately 66 days or more, approximately 66 days or more, approximately 66 days or more, approximately 66 days or more, approximately 66 days or more, approximately 67 days or more, approximately 68 days or more, approximately 69 days or more, approximately 70 days or more, approximately 77 days or more, approximately 77 days or more, approximately 77 days or more, approximately 77 days or more, approximately 77 days or more, approximately 77 days or more, approximately 77 days or more, approximately 78 days or more, approximately 79 days or more, approximately 80 days or more, approximately 88 days or more, approximately 88 days or more, approximately 88 days or more, approximately 88 days or more, approximately 88 days or more, approximately 88 days or more, approximately 88 days or more, approximately 88 days or more, approximately 89 days or more, or approximately 90 days or more after administration of the multi-component cellular therapy; and/or aa) overall survival increases after administration of the multi-component cellular therapy, relative to a corresponding human subject that has been administered a standard myeloablative allogeneic hematopoietic stem cell transplant (alloHSCT), optionally wherein the human subject experiences overall survival for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or bb) incidence of re-hospitalization decreases after administration of the multi-component cellular therapy, relative to a corresponding human subject that has been administered a standard myeloablative allogeneic hematopoietic stem cell transplant (alloHSCT), optionally wherein the human subject does not require re-hospitalization for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy; and/or cc) the human subject is approximately 3 months of age or older, or the human subject is from between approximately 3 months to approximately 18 years of age, or the human subject is approximately 18 years of age or older, or the human subject is between approximately 18 years to approximately 65 years of age, or the human subject is between approximately 18 years to approximately 75 years of age, or the human subject is from approximately 66 years of age to approximately 75 years of age, or the human subject is from between approximately 3 months to approximately 75 years of age; and/or dd) the human subject has received from one to five previous lines of therapy.

Other aspects of the present disclosure relate to a cellular therapy kit comprising the multi-component cellular therapy product of any of the preceding embodiments, optionally wherein the kit further comprises written instructions for using the cellular therapy for treating a hematologic malignancy in a human subject, optionally wherein the hematologic malignancy is selected from: leukemia, acute leukemia, chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN), optionally wherein the leukemia is active leukemia, optionally wherein the acute leukemia is in complete remission or in complete remission with incomplete hematologic recovery, optionally wherein minimal residual disease is present in the human subject or minimal residual disease is absent in the human subject, optionally wherein the acute leukemia is categorized as intermediate-risk to very high-risk acute leukemia, optionally wherein the acute leukemia is having primary refractory acute leukemia or acute leukemia with minimal residual disease, optionally wherein the acute leukemia is acute myeloid leukemia (AML), acute lymphoid leukemia (ALL), and/or mixed phenotype acute leukemia (MPAL), optionally wherein the AML is low-risk AML to high-risk AML, optionally wherein the high-risk AML comprises a complex karyotype with 3 or more clonal chromosomal abnormalities selected from: monosomal karyotype-5, 5q-, -7 or 7q-, t(11q23, t(9;11), inv(3), t(3,3)t(6;9)t(9;22), normal karyotype with a fms-like tyrosine kinase 3 (FLT3)-ITD mutation, and any combination thereof, optionally wherein the CML is in blast phase, is in second chronic phase, is in chronic phase, is accelerated, has a history of blast crisis, and/or is intolerant of one or more first-generation or second-generation tyrosine kinase inhibitors (TKIs), optionally wherein the myelodysplastic syndrome is categorized as high-risk, optionally wherein the myelodysplastic syndrome is therapy-related myelodysplastic syndrome and/or secondary myelodysplastic syndrome, optionally wherein the non-Hodgkin lymphoma is non-Hodgkin lymphoma with poor risk features not suitable for autologous hematopoietic cell transplant (HCT).

It shall be understood that different aspects and/or embodiments of the present disclosure can be appreciated individually, collectively, or in combination with each other. Any description herein concerning a specific composition, multi-component pharmaceutical treatment, multi-component cellular therapy product, cell population, solution, formulation, kit, and/or method apply to and may be used for any other specific composition, multi-component pharmaceutical treatment, multi-component cellular therapy product, cell population, solution, formulation, kit, and/or method. Additionally, any composition disclosed herein is applicable to any herein-disclosed method. In other words, any aspect or embodiment described herein can be combined with any other aspect or embodiment as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIGS. 1A-B illustrate the schematics of the transplant according to the methods described herein (identified as High-Precision Orca-T or Orca-T) and the differences compared to a standard of care (SOC) cohort (identified as Conventional Transplant or SOC).

FIG. 1C illustrates a schematic of graft production and administration.

FIG. 2A illustrates the weight of patients enrolled in the study disclosed in the Examples.

FIGS. 2B-2C illustrate the HSPC and Treg cell dose administered to the patients enrolled in the study disclosed in the Examples.

FIG. 2D illustrates the purity of Treg cells administered to the patients enrolled in the study disclosed in the Examples.

FIG. 3A shows the time to platelet engraftment in the study group (identified as Orca-T) and the standard of care (SOC) cohort.

FIGS. 3B-3L illustrate engraftment of various cell populations in the patients in the study group disclosed in the Examples. The figures also illustrate the levels of each cell type in the donors before sample collection. Boxplots where shown: boxes show the 75th, 50th, and 25th percentiles; whiskers show the 90th and 10th percentiles. X-axes nomenclature: the leading number (e.g., 01, 02, 025, . . . ) are mentioned for ordering; following the underscore, Dscrn=healthy donor pre-G-CSF mobilization, Rscrn=recipient within 1 month prior to conditioning, apher=healthy donor blood draw at the time of apheresis, d028=recipient day 28 post-transplant, d056-d365=recipient days post-transplant. N's shown indicate the sample sizes for each timepoint. Symbols indicate values for individual measurements. Cell numbers ×10⁻³ per uL of blood are equivalent to ×1,000 cells per uL of blood.

FIG. 3M-3N show the timeline of lymphocyte and monocyte engraftment in a subset of the study group (Orca-T) and the standard of care cohort.

FIG. 3O shows representative flow cytometry data for the frequency of CD3⁺CD4⁺ T cells that were Tregs in two subjects compared to a healthy control. In the healthy control, 3.72% of circulating CD3⁺CD4⁺ T cells were Tregs (CD25⁺ CD127^{dim}). In the two graft recipients, 28.1% and 23.7% of CD3⁺CD4⁺ T cells were Tregs on day +28, 32.3% and 17.8% on day +56, and 19.2% and 20.7% on day +100 post-transplant.

FIG. 3P shows flow cytometry data for B cell markers from a sample from a recipient of a composition of the disclosure compared to a healthy control. In all cases, the Y axis is for CD19⁺ staining. The left panels show gating of lymphocytes to identify B cells (CD19⁺) and T cells (CD3⁺). 13.4% of lymphocytes in the graft recipient were B cells, compared to 9.84% in the healthy control. The second from left panels show that 98.3-100% of cells gates as CD19⁺ were also CD20⁺. The panels second from the right show the fraction of B cells that are IgD⁺, which can be used to identify mature B cells. 92.1% of B cells in the graft recipient were IgD⁺, and 89.5% in the healthy control. The right-most panels show staining for CD27, which can be used to identify memory B cells, late plasmablasts, and plasma cells, for example. 43.6% of B cells in the graft recipient were CD27⁺, and 67.1% in the healthy control.

FIG. 4A shows the onset of grade ≥2 aGVHD in the study group (Orca-T) and the standard of care cohort through day +120 post-transplant. At nearly all timepoints, Orca-T data is below the standard of care data.

FIG. 4B shows the onset of grade ≥3 aGVHD in the study group (Orca T) and the standard of care cohort through day +120 post-transplant. At nearly all timepoints, Orca-T data is below the standard of care data.

FIG. 4C shows the onset of moderate to severe cGVHD in the study group (Orca-T) and the standard of care (SOC) cohort through day +365 post-transplant. At nearly all timepoints, Orca-T data is below the standard of care data.

FIG. 4D shows the non-relapse related mortality in the study group (Orca-T) and the standard of care (SOC) cohort through day +365 post-transplant. At nearly all timepoints, Orca-T data is below the standard of care data.

FIG. 4E shows relapse rates in the study group (Orca-T) and the standard of care cohort through day +365 post-transplant. At the final timepoint, Orca-T relapse rate is 16% and the standard of care relapse rate is 19%.

FIG. 4F shows GVHD and relapse-free survival rates in the study group (Orca-T) and the standard of care cohort through day +365 post-transplant. At nearly all timepoints, Orca-T data is above the standard of care data.

FIG. 4G shows cGVHD-free survival rates in the study group and the standard of care cohort through day +365 post-transplant. At nearly all timepoints, Orca-T data is above the standard of care data.

FIG. 4H shows overall survival rates in the study group and the standard of care cohort through day +365 post-transplant. At the final timepoint, Orca-T overall survival rate is 90% and the standard of care overall survival rate is 78%. FIG. 4I shows hospitalization days in a subset of the study group and the standard of care (SOC) cohort through day +365 post-transplant.

FIG. 5 summarizes the disease status of a small subset of subjects in the study group before transplant and at day +90, +180, and +356 post-transplant. CR signifies complete remission, MRD signifies minimal residual disease.

FIGS. 6A-6F compare the aGVHD, cGVHD, relapse, relapse-free survival, GVHD and relapse free survival (GRFS) and overall survival rates in a subset of the patients in the study group that received different conditioning regimens.

FIGS. 7A-7H compare the aGVHD, cGVHD, non-relapse related mortality, relapse, relapse-free survival, GVHD and relapse free survival (GRFS) and overall survival rates in a subset of the patients in the study group that received different GVHD prophylactic agents.

FIGS. 8A-8C illustrate aGVHD and cGVHD rates in patients with different serum tacrolimus trough levels.

FIGS. 9A-9B compare the aGVHD and cGVHD levels in patients that had different serum tacrolimus levels.

FIGS. 9C-9D compare the aGVHD and cGVHD levels in patients that had different serum tacrolimus levels but were given the same conditioning regimen of busulfan and cyclophosphamide (Bu/Cy).

FIGS. 9E-9G compare the aGVHD and cGVHD levels in patients that had different serum tacrolimus levels but were given the same conditioning regimen of Total Body Irradiation (TBI)/Busulfan, Fludarabine, Thiotepa (TBI/BFT).

FIG. 9H shows the average trough tacrolimus level through day +30 post-transplant, plotted against the proportion of CD3⁺ cells of donor origin at day +30 (except that chimerism data is from day 90 where indicated by “D90”).

FIG. 10A illustrates neutrophil and platelet engraftment has been rapid following Orca-T plus single-agent GVHD prophylaxis. Neutrophil and platelet engraftment occurred at median of 13 and 15 days, respectively. Two graft failure events (1.4%) have been reported out of 137 patients treated with Orca-T. FIG. 10B illustrates further neutrophil and platelet engraftment data, showing that Neutrophil engraftment occurred at median of 13 days and platelet engraftment occurred at median of 19 days.

FIG. 11 illustrates Acute GVHD results which is uncommon with Orca-T despite a limited GVHD prophylaxis regimen. The rates of Grade ≥2 and Grade ≥3 aGVHD were 17% and 4% (graded per MAGIC criteria), respectively, through Day +180 with Orca-T plus single-agent GVHD prophylaxis. Only 4 patients (3%) experienced steroid-refractory or steroid-dependent aGVHD.

FIG. 12 illustrates moderate/severe chronic GVHD is rare with Orca-T. The rate of NIH moderate to severe cGVHD was 5% through 1-year post-transplant.

FIG. 13 describes that despite reduced GVHD rates, relapse rates following Orca-T are not elevated compared to historic controls, suggesting that graft-vs-leukemia effects remain intact with Orca-T. Of note, patients with active leukemia at time of transplant were allowed to enroll on Orca-T studies provided that bone marrow blast burden was ≤10%. 14% of patients enrolled had active leukemia at transplant; similarly, 16% of the comparator cohort had “advanced” disease.

FIG. 14 illustrates that the addition of thiotepa to busulfan-based conditioning regimens decreases relapse without increasing non-relapse mortality with Orca-T. Relapse-free survival through 1 year is shown for all Orca-T patients (n=137), Bu/Flu or Bu/Cy conditioning prior to Orca-T (n=50), and Bu/Flu/Thiotepa conditioning prior to Orca-T (n=49).

FIG. 15 illustrates lymphocyte subset reconstitution. Shown are the absolute cell counts per microliter of whole blood of T cells, CD4⁺ T cells, CD8⁺ T cells, regulatory T cells, B cells, and NK cells in recipients pre-transplant and during the first-year post-transplant. The number of patients included at each time point is indicated in each graph. Blue circles and bars show the median and quartile values.

FIG. 16 illustrates severe infection which was found to be uncommon in patients treated with Orca-T. Shown is the incidence of Grade 3 infections using the BMT-CTN MOP V4.0 grading scale. COVID-19/SARS-COV2 infections were reported in 13 patients and resulted in one death.

FIG. 17 illustrates Orca-T plus single-agent GVHD prophylaxis results in GVHD and relapse-free survival (GRFS) at 1 year which was found to be superior to historic comparators. Modified GRFS is shown: survival free of Grade 3-4 aGVHD, moderate to severe cGVHD, and relapse.

FIG. 18 illustrates preliminary overall survival. Data suggests that Orca-T may lead to improved survival in patients undergoing allogenic hematopoietic stem cell transplant (alloHSCT) for high-risk hematologic malignancies.

FIG. 19A illustrates neutrophil and platelet engraftment using Orca-T derived from donors that are 7/8 HLA mismatched. Neutrophil and platelet engraftment occurred at median of 12.5 and 15.5 days, respectively. FIG. 19B illustrates percent chimerism of whole blood and T-cell using Orca-T derived from donors that are 7/8 HLA mismatched. Whole blood exhibited full chimerism and T-cells exhibited over 90% chimerism 90 days post-transplant.

FIG. 20A illustrates IL-2 serum plasma levels 14 days post-transplant using Orca-T derived from donors that are 7/8 HLA mismatched or 8/8 matched. FIG. 20B illustrates IL-10 serum plasma levels 14 days post-transplant using Orca-T derived from donors that are 7/8 HLA mismatched or 8/8 matched.

FIG. 21 illustrates clinical outcomes of six patients that received Orca-T derived from donors that are 7/8 HLA mismatched at 90 days, 180 days, and 365 days post-transplant.

FIG. 22A illustrates percent relapse-free survival in patients receiving BFT conditioning regimens prior to Orca-T. FIG. 22B illustrates percent non-relapse mortality in patients receiving BFT conditioning regimens prior to Orca-T. Median follow-up time was 413 days.

FIG. 23 illustrates percent relapse-free survival by MRD in patients receiving BFT conditioning regimens prior to Orca-T. Median follow-up time was 413 days.

FIG. 24 illustrates incidence of Grade 3+ infections (using the BMT-CTN MOP V4.0 grading scale) in patients receiving BFT conditioning regimens prior to Orca-T.

FIG. 25A illustrates the incidence of grade 3 or higher acute GVHD (aGVHD) in patients receiving BFT conditioning regimens prior to Orca-T. FIG. 25B illustrates the incidence of moderate to severe chronic GVHD (cGVHD) in patients receiving BFT conditioning regimens prior to Orca-T.

FIG. 26A illustrates the percentage of acute grade 3-4, chronic moderate to severe GVHD relapse free survival (GRFS) at 1 year in patients receiving BFT conditioning regimens prior to Orca-T. FIG. 26B illustrates the overall survival at 1 year in patients receiving BFT conditioning regimens prior to Orca-T.

### DETAILED DESCRIPTION

### Introduction

Various embodiments of the present disclosure provide compositions, multi-component pharmaceutical treatments, multi-component cellular therapy products, cell populations, solutions, formulations, kits, and/or methods relating to improved allogeneic hematopoietic stem cell transplantation (alloHSCT) that includes transplantation of distinct cell populations that are enriched for hematopoietic stem and progenitor cells (HSPCs), regulatory T-cells (Tregs), and conventional T-cells (Tcons).

Standard alloHSCT is the transplantation of multipotent hematopoietic stem and progenitor cells (HSPCs), usually derived from donor bone marrow, peripheral blood, or umbilical cord blood, into a recipient. The recipient can be subjected to myeloablative conditioning, which kills hematopoietic cells including tumor cells and host immune cells. The HSPCs transplanted into the recipient then reconstitutes the hematopoietic compartment. HSCT can be useful as a treatment for cancer due to the ability of donor T cells to exert anti-tumor effects, referred to as graft versus tumor (GVT). In patients with hematologic malignancies that are refractory to chemotherapy, HSCT is associated with improved survival.

Surprisingly, recipients of the improved alloHSCT of the present disclosure who received a single agent graft versus host disease (GVHD) prophylactic agent, such as tacrolimus, had significantly better clinical outcomes than existing standard alloHSCT regimens and standards of care. These recipients experience improved clinical outcomes including, for example, increased overall survival, increased relapse-free survival, increased GVHD- and relapse-free survival (GRFS), more rapid and/or complete engraftment of various hematopoietic components (e.g., neutrophils, platelets, T cells, B cells), improved donor chimerism (e.g., T cell chimerism), decreased relapse, decreased primary graft failure, decreased secondary graft failure, decreased treatment-associated mortality, reduced acute and/or chronic GVHD, and shorter time to discharge from hospital following the single agent GVHD prophylactic agent, such as tacrolimus

Although alloHSCT is associated with improved survival in patients with hematologic malignancies that are refractory to chemotherapy, some subjects treated with existing standard alloHSCT regimens exhibit cancer relapse, and a number of complications can limit the efficacy of standard alloHSCT. The effectiveness of standard alloHSCT can be limited by, for example, primary graft failure, secondary graft failure, limited or slow engraftment of various hematopoietic components (e.g., neutrophils, platelets, T cells, or B cells), and limited donor chimerism (e.g., T cell chimerism). Additionally, standard alloHSCT can cause treatment-associated morality or toxicity, for example, However, donor T cells can also attack non-tumor host cells, resulting in graft versus host disease (GVHD). GVHD is a major source of post-HCT complications and can be fatal. Management of GVHD can require immunosuppressive therapy or cytotoxic mediations, which can cause toxicity, increase susceptibility to infection, and/or blunt anti-tumor immunity. The early morbidity and mortality associated with acute graft versus host disease (aGVHD; which occurs within the first 100 days post-transplant) is a major factor limiting the success of HCT, as is the long-term morbidity associated with chronic GVHD (cGVHD). GVHD is a risk for both HLA-matched and HLA-mismatched transplantations. GVHD can occur even if the donor and recipient are HLA-matched, because the immune system can still recognize other differences between in the donor tissues.

Both GVT and GVHD are largely mediated by conventional T cells (Tcons), which mount immune responses upon recognition of cognate antigen by T cell receptors. Depleting T cells from hematopoietic stem cell transplantation (HCT) grafts can reduce GVHD, but can also result in reduced GVT and increased likelihood of cancer relapse. Besides Tcons, Tregs are an additional subset of T cells that negatively regulate inflammation and that promote immune tolerance. Tregs can prevent or reduce GVHD through their negative regulation of inflammation, including, for example, inflammation elicited by donor Tcons when they recognize recipient antigens.

Provided herein are compositions and methods for improved alloHISCT, comprising administering to a subject certain cell populations that comprise populations of cells, including a first population of CD45⁺ cells that comprise, at least, HSPCs, a second population of CD45⁺ cells that comprise, at least Tregs, and a third population of CD45⁺ cells that comprises, at least, Tcons. In some embodiments, the second population of CD45⁺ cells is also referred to a cell population enriched for Tregs. Without wishing to be bound by theory, administering the second population of CD45⁺ cells reduces the incidence and/or severity of GVHD, while administering the third population of CD45⁺ cells, which comprises Tcons, enhances GVT. Thus, embodiments of the present disclosure provide a provides compositions, multi-component pharmaceutical treatments, multi-component cellular therapy products, cell populations, solutions, formulations, kits, and/or methods for administering, at least, both populations of T cells, to enhance GVT while minimizing GVHD. Accordingly, the compositions, multi-component pharmaceutical treatments, multi-component cellular therapy products, cell populations, solutions, formulations, kits, and/or methods disclosed herein can retain the graft-versus-tumor (GVT) effects of alloHSCT administered to a subject having a cancer (e.g., a hematologic cancer), while preventing or reducing graft versus host disease (GVHD) in the subject. In some embodiments, two or more populations of cells are administered at different times, for example, first population of CD45⁺ cells that comprises, at least, HSPCs and the cell population enriched for Tregs can be administered prior to the third population of CD45⁺ cells that comprises, at least, Tcons.

### Cell Populations

Embodiments of the present disclosure provide a multi-component pharmaceutical treatment or multi-component cellular therapy product to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent, e.g., tacrolimus. In various embodiments, the HSPCs are CD34⁺. In some embodiments, the multi-component cellular therapy product comprises a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, from approximately 1.5×10⁷ to approximately 1.5×10¹⁰ HSPCs, or from approximately 5.0×10⁵ to approximately 5.0×10⁸ HSPCs, wherein the first population of CD45⁺ cells is formulated with an excipient at a neutral pH; b) a second single dose transfer bag comprising a second population of isolated CD45⁺ cells comprising a dose of approximately 1.0×10⁵ to approximately 2.0×10⁷ fresh CD4⁺ CD25⁺ CD127^{dim} regulatory T cells (Tregs) per kilogram of body weight of the human subject receiving the product, from approximately 1.5×10⁷ to approximately 3.0×10⁹ fresh Tregs, or from approximately 5.0×10⁵ to approximately 1.0×10⁸ fresh Tregs, wherein the second population of isolated CD45⁺ cells is formulated with an excipient at a neutral pH; and c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons, wherein the third population of isolated CD45⁺ cells is formulated with an excipient at a neutral pH, wherein the excipient comprises one more cryoprotectants.

In some embodiments, the first population of CD45⁺ cells comprises HSPCs. In other embodiments, the first population of CD45⁺ cells comprises at least one dose of HSPCs. In some embodiments, the first population of CD45⁺ cells comprising HSPCs, or comprising at least one dose of HSPCs, comprises from approximately 1.0×10⁵ to approximately 5.0×10⁸ HSPCs per kilogram of body weight of the human subject, from approximately 1.0×10⁵ to approximately 1.0×10⁸ HSPCs per kilogram of body weight of the human subject, or from approximately 5.0×10⁵ to approximately 2.0×10⁷ HSPCs per kilogram of body weight of the human subject. In some embodiments, the first population of CD45⁺ cells comprising HSPCs, or comprising at least one dose of HSPCs, comprises approximately 1.0×10⁵ or more HSPCs per kilogram of body weight of the human subject, approximately 1.5×10⁵ or more HSPCs per kilogram of body weight of the human subject, approximately 2.0×10⁵ or more HSPCs per kilogram of body weight of the human subject, approximately 2.5×10⁵ or more HSPCs per kilogram of body weight of the human subject, approximately 3.0×10⁵ or more HSPCs per kilogram of body weight of the human subject, approximately 3.5×10⁵ or more HSPCs per kilogram of body weight of the human subject, approximately 4.0×10⁵ or more HSPCs per kilogram of body weight of the human subject, approximately 4.5×10⁵ or more HSPCs per kilogram of body weight of the human subject, approximately 5.0×10⁵ or more HSPCs per kilogram of body weight of the human subject, approximately 5.5×10⁵ or more HSPCs per kilogram of body weight of the human subject, approximately 6.0×10⁵ or more HSPCs per kilogram of body weight of the human subject, approximately 6.5×10⁵ or more HSPCs per kilogram of body weight of the human subject, approximately 7.0×10⁵ or more HSPCs per kilogram of body weight of the human subject, approximately 7.5×10⁵ or more HSPCs per kilogram of body weight of the human subject, approximately 8.0×10⁵ or more HSPCs per kilogram of body weight of the human subject, approximately 8.5×10⁵ or more HSPCs per kilogram of body weight of the human subject, approximately 9.0×10⁵ or more HSPCs per kilogram of body weight of the human subject, approximately 9.5×10⁵ or more HSPCs per kilogram of body weight of the human subject, approximately 1.0×10⁶ or more HSPCs per kilogram of body weight of the human subject, approximately 1.25×10⁶ or more HSPCs per kilogram of body weight of the human subject, approximately 1.5×10⁶ or more HSPCs per kilogram of body weight of the human subject, approximately 1.75×10⁶ or more HSPCs per kilogram of body weight of the human subject, approximately 2.0×10⁶ or more HSPCs per kilogram of body weight of the human subject, approximately 2.25×10⁶ or more HSPCs per kilogram of body weight of the human subject, approximately 2.5×10⁶ or more HSPCs per kilogram of body weight of the human subject, approximately 2.75×10⁶ or more HSPCs per kilogram of body weight of the human subject, approximately 3.0×10⁶ or more HSPCs per kilogram of body weight of the human subject, approximately 3.25×10⁶ or more HSPCs per kilogram of body weight of the human subject, approximately 3.5×10⁶ or more HSPCs per kilogram of body weight of the human subject, approximately 3.75×10⁶ or more HSPCs per kilogram of body weight of the human subject, approximately 4.0×10⁶ or more HSPCs per kilogram of body weight of the human subject, approximately 4.25×10⁶ or more HSPCs per kilogram of body weight of the human subject, approximately 4.5×10⁶ or more HSPCs per kilogram of body weight of the human subject, approximately 4.75×10⁶ or more HSPCs per kilogram of body weight of the human subject, approximately 5.0×10⁶ or more HSPCs per kilogram of body weight of the human subject, approximately 5.25×10⁶ or more HSPCs per kilogram of body weight of the human subject, approximately 5.5×10⁶ or more HSPCs per kilogram of body weight of the human subject, approximately 5.75×10⁶ or more HSPCs per kilogram of body weight of the human subject, approximately 6.0×10⁶ or more HSPCs per kilogram of body weight of the human subject, approximately 6.25×10⁶ or more HSPCs per kilogram of body weight of the human subject, approximately 6.5×10⁶ or more HSPCs per kilogram of body weight of the human subject, approximately 6.75×10⁶ or more HSPCs per kilogram of body weight of the human subject, approximately 7.0×10⁶ or more HSPCs per kilogram of body weight of the human subject, approximately 7.25×10⁶ or more HSPCs per kilogram of body weight of the human subject, approximately 7.5×10⁶ or more HSPCs per kilogram of body weight of the human subject, approximately 7.75×10⁶ or more HSPCs per kilogram of body weight of the human subject, approximately 8.0×10⁶ or more HSPCs per kilogram of body weight of the human subject, approximately 8.25×10⁶ or more HSPCs per kilogram of body weight of the human subject, approximately 8.5×10⁶ or more HSPCs per kilogram of body weight of the human subject, approximately 8.75×10⁶ or more HSPCs per kilogram of body weight of the human subject, approximately 9.0×10⁶ or more HSPCs per kilogram of body weight of the human subject, approximately 9.25×10⁶ or more HSPCs per kilogram of body weight of the human subject, approximately 9.5×10⁶ or more HSPCs per kilogram of body weight of the human subject, approximately 9.75×10⁶ or more HSPCs per kilogram of body weight of the human subject, approximately 1.0×10⁷ or more HSPCs per kilogram of body weight of the human subject, approximately 1.25×10⁷ or more HSPCs per kilogram of body weight of the human subject, approximately 1.5×10⁷ or more HSPCs per kilogram of body weight of the human subject, approximately 1.75×10⁷ or more HSPCs per kilogram of body weight of the human subject, approximately 2.0×10⁷ or more HSPCs per kilogram of body weight of the human subject, approximately 2.25×10⁷ or more HSPCs per kilogram of body weight of the human subject, approximately 2.5×10⁷ or more HSPCs per kilogram of body weight of the human subject, approximately 2.75×10⁷ or more HSPCs per kilogram of body weight of the human subject, approximately 3.0×10⁷ or more HSPCs per kilogram of body weight of the human subject, approximately 3.25×10⁷ or more HSPCs per kilogram of body weight of the human subject, approximately 3.5×10⁷ or more HSPCs per kilogram of body weight of the human subject, approximately 3.75×10⁷ or more HSPCs per kilogram of body weight of the human subject, approximately 4.0×10⁷ or more HSPCs per kilogram of body weight of the human subject, approximately 4.25×10⁷ or more HSPCs per kilogram of body weight of the human subject, approximately 4.5×10⁷ or more HSPCs per kilogram of body weight of the human subject, approximately 4.75×10⁷ or more HSPCs per kilogram of body weight of the human subject, approximately 5.0×10⁷ or more HSPCs per kilogram of body weight of the human subject, approximately 5.25×10⁷ or more HSPCs per kilogram of body weight of the human subject, approximately 5.5×10⁷ or more HSPCs per kilogram of body weight of the human subject, approximately 5.75×10⁷ or more HSPCs per kilogram of body weight of the human subject, approximately 6.0×10⁷ or more HSPCs per kilogram of body weight of the human subject, approximately 6.25×10⁷ or more HSPCs per kilogram of body weight of the human subject, approximately 6.5×10⁷ or more HSPCs per kilogram of body weight of the human subject, approximately 6.75×10⁷ or more HSPCs per kilogram of body weight of the human subject, approximately 7.0×10⁷ or more HSPCs per kilogram of body weight of the human subject, approximately 7.25×10⁷ or more HSPCs per kilogram of body weight of the human subject, approximately 7.5×10⁷ or more HSPCs per kilogram of body weight of the human subject, approximately 7.75×10⁷ or more HSPCs per kilogram of body weight of the human subject, approximately 8.0×10⁷ or more HSPCs per kilogram of body weight of the human subject, approximately 8.25×10⁷ or more HSPCs per kilogram of body weight of the human subject, approximately 8.5×10⁷ or more HSPCs per kilogram of body weight of the human subject, approximately 8.75×10⁷ or more HSPCs per kilogram of body weight of the human subject, approximately 9.0×10⁷ or more HSPCs per kilogram of body weight of the human subject, approximately 9.25×10⁷ or more HSPCs per kilogram of body weight of the human subject, approximately 9.5×10⁷ or more HSPCs per kilogram of body weight of the human subject, approximately 9.75×10⁷ or more HSPCs per kilogram of body weight of the human subject, approximately 1.0×10⁸ or more HSPCs per kilogram of body weight of the human subject, approximately 1.25×10⁸ or more HSPCs per kilogram of body weight of the human subject, approximately 1.5×10⁸ or more HSPCs per kilogram of body weight of the human subject, approximately 1.75×10⁸ or more HSPCs per kilogram of body weight of the human subject, approximately 2.0×10⁸ or more HSPCs per kilogram of body weight of the human subject, approximately 2.25×10⁸ or more HSPCs per kilogram of body weight of the human subject, approximately 2.5×10⁸ or more HSPCs per kilogram of body weight of the human subject, approximately 2.75×10⁸ or more HSPCs per kilogram of body weight of the human subject, approximately 3.0×10⁸ or more HSPCs per kilogram of body weight of the human subject, approximately 3.25×10⁸ or more HSPCs per kilogram of body weight of the human subject, approximately 3.5×10⁸ or more HSPCs per kilogram of body weight of the human subject, approximately 3.75×10⁸ or more HSPCs per kilogram of body weight of the human subject, approximately 4.0×10⁸ or more HSPCs per kilogram of body weight of the human subject, approximately 4.25×10⁸ or more HSPCs per kilogram of body weight of the human subject, approximately 4.5×10⁸ or more HSPCs per kilogram of body weight of the human subject, approximately 4.75×10⁸ or more HSPCs per kilogram of body weight of the human subject, or approximately 5.0×10⁸ or more HSPCs per kilogram of body weight of the human subject. In some embodiments, the body weight of the human subject is actual body weight of the human subject. In other embodiments, the body weight of the human subject is ideal actual body weight of the human subject.

In embodiments, the first population of CD45⁺ cells comprises at least about 0.5% granulocytes, at least about 1% granulocytes, at most about 5% granulocytes, at most about 3% granulocytes, at most about 3% monocytes, at most about 2% monocytes, at most about 0.5% lymphocytes, at most about 2% lymphocytes, at least about 15% granulocytes, at least about 20% granulocytes, at most about 35% granulocytes, at most about 30% granulocytes, at most about 25% granulocytes, at least about 15% monocytes, at least about 20% monocytes, at most about 35% monocytes, at most about 30% monocytes, at most about 25% monocytes, at least about 0.5% NK cells, and or at least about 2% NK cells. In various embodiments, the first population of CD45⁺ cells, the population of cells enriched for Tregs, and the third population of CD45⁺ cells are obtained from a single donor. In some embodiments, the first population of CD45⁺ cells, the population of cells enriched for Tregs, and/or the third population of CD45⁺ cells is allogeneic relative to the human subject. In embodiments, the first population of CD45⁺ cells, the population of cells enriched for Tregs, and/or the third population of CD45⁺ cells is obtained from a donor that is HLA-matched relative to the human subject. In various embodiments, the first population of CD45⁺ cells, the population of cells enriched for Tregs, and/or the third population of CD45⁺ cells is obtained from a donor that is HLA-mismatched relative to the human subject. In some embodiments, the first population of CD45⁺ cells, the population of cells enriched for Tregs, and/or the third population of CD45⁺ cells is obtained from a donor that is haploidentical relative to the human subject.

In some embodiments, the Tregs are CD4⁺ CD25⁺ CD127^{dim} or CD4⁺ FOXP3⁺. In some embodiments, the Tregs are CD4⁺CD25⁺CD127^{dim} and are also FOXP3⁺. In some cases, the population of cells enriched for Tregs comprises CD45⁺ cells, e.g., more than about 90% of the CD45⁺ cells are Tregs. In other embodiments, the second population of CD45⁺ cells comprises one or more doses of Tregs. In various embodiments, the population of cells enriched for Tregs, or the second population of CD45⁺ cells comprising at least one dose of Tregs, comprises from approximately 1.0×10⁵ to approximately 1.0×10⁸ Tregs per kilogram of body weight of the human subject, from approximately 1.0×10⁵ to approximately 2.0×10⁷ Tregs per kilogram of body weight of the human subject, from approximately 1.0×10⁵ to approximately 1.0×10⁷ Tregs per kilogram of body weight of the human subject, or from approximately 5.0×10⁵ to approximately 4.0×10⁶ Tregs per kilogram of body weight of the human subject. In some embodiments, the population of cells enriched for Tregs, or the second population of CD45⁺ cells comprising at least one dose of Tregs, comprises approximately 1.0×10⁵ or more Tregs per kilogram of body weight of the human subject, approximately 1.5×10⁵ or more Tregs per kilogram of body weight of the human subject, approximately 2.0×10⁵ or more Tregs per kilogram of body weight of the human subject, approximately 2.5×10⁵ or more Tregs per kilogram of body weight of the human subject, approximately 3.0×10⁵ or more Tregs per kilogram of body weight of the human subject, approximately 3.5×10⁵ or more Tregs per kilogram of body weight of the human subject, approximately 4.0×10⁵ or more Tregs per kilogram of body weight of the human subject, approximately 4.5×10⁵ or more Tregs per kilogram of body weight of the human subject, approximately 5.0×10⁵ or more Tregs per kilogram of body weight of the human subject, approximately 5.5×10⁵ or more Tregs per kilogram of body weight of the human subject, approximately 6.0×10⁵ or more Tregs per kilogram of body weight of the human subject, approximately 6.5×10⁵ or more Tregs per kilogram of body weight of the human subject, approximately 7.0×10⁵ or more Tregs per kilogram of body weight of the human subject, approximately 7.5×10⁵ or more Tregs per kilogram of body weight of the human subject, approximately 8.0×10⁵ or more Tregs per kilogram of body weight of the human subject, approximately 8.5×10⁵ or more Tregs per kilogram of body weight of the human subject, approximately 9.0×10⁵ or more Tregs per kilogram of body weight of the human subject, approximately 9.5×10⁵ or more Tregs per kilogram of body weight of the human subject, approximately 1.0×10⁶ or more Tregs per kilogram of body weight of the human subject, approximately 1.25×10⁶ or more Tregs per kilogram of body weight of the human subject, approximately 1.5×10⁶ or more Tregs per kilogram of body weight of the human subject, approximately 1.75×10⁶ or more Tregs per kilogram of body weight of the human subject, approximately 2.0×10⁶ or more Tregs per kilogram of body weight of the human subject, approximately 2.25×10⁶ or more Tregs per kilogram of body weight of the human subject, approximately 2.5×10⁶ or more Tregs per kilogram of body weight of the human subject, approximately 2.75×10⁶ or more Tregs per kilogram of body weight of the human subject, approximately 3.0×10⁶ or more Tregs per kilogram of body weight of the human subject, approximately 3.25×10⁶ or more Tregs per kilogram of body weight of the human subject, approximately 3.5×10⁶ or more Tregs per kilogram of body weight of the human subject, approximately 3.75×10⁶ or more Tregs per kilogram of body weight of the human subject, approximately 4.0×10⁶ or more Tregs per kilogram of body weight of the human subject, approximately 4.25×10⁶ or more Tregs per kilogram of body weight of the human subject, approximately 4.5×10⁶ or more Tregs per kilogram of body weight of the human subject, approximately 4.75×10⁶ or more Tregs per kilogram of body weight of the human subject, approximately 5.0×10⁶ or more Tregs per kilogram of body weight of the human subject, approximately 5.25×10⁶ or more Tregs per kilogram of body weight of the human subject, approximately 5.5×10⁶ or more Tregs per kilogram of body weight of the human subject, approximately 5.75×10⁶ or more Tregs per kilogram of body weight of the human subject, approximately 6.0×10⁶ or more Tregs per kilogram of body weight of the human subject, approximately 6.25×10⁶ or more Tregs per kilogram of body weight of the human subject, approximately 6.5×10⁶ or more Tregs per kilogram of body weight of the human subject, approximately 6.75×10⁶ or more Tregs per kilogram of body weight of the human subject, approximately 7.0×10⁶ or more Tregs per kilogram of body weight of the human subject, approximately 7.25×10⁶ or more Tregs per kilogram of body weight of the human subject, approximately 7.5×10⁶ or more Tregs per kilogram of body weight of the human subject, approximately 7.75×10⁶ or more Tregs per kilogram of body weight of the human subject, approximately 8.0×10⁶ or more Tregs per kilogram of body weight of the human subject, approximately 8.25×10⁶ or more Tregs per kilogram of body weight of the human subject, approximately 8.5×10⁶ or more Tregs per kilogram of body weight of the human subject, approximately 8.75×10⁶ or more Tregs per kilogram of body weight of the human subject, approximately 9.0×10⁶ or more Tregs per kilogram of body weight of the human subject, approximately 9.25×10⁶ or more Tregs per kilogram of body weight of the human subject, approximately 9.5×10⁶ or more Tregs per kilogram of body weight of the human subject, approximately 9.75×10⁶ or more Tregs per kilogram of body weight of the human subject, approximately 1.0×10⁷ or more Tregs per kilogram of body weight of the human subject, approximately 1.25×10⁷ or more Tregs per kilogram of body weight of the human subject, approximately 1.5×10⁷ or more Tregs per kilogram of body weight of the human subject, approximately 1.75×10⁷ or more Tregs per kilogram of body weight of the human subject, approximately 2.0×10⁷ or more Tregs per kilogram of body weight of the human subject, approximately 2.25×10⁷ or more Tregs per kilogram of body weight of the human subject, approximately 2.5×10⁷ or more Tregs per kilogram of body weight of the human subject, approximately 2.75×10⁷ or more Tregs per kilogram of body weight of the human subject, approximately 3.0×10⁷ or more Tregs per kilogram of body weight of the human subject, approximately 3.25×10⁷ or more Tregs per kilogram of body weight of the human subject, approximately 3.5×10⁷ or more Tregs per kilogram of body weight of the human subject, approximately 3.75×10⁷ or more Tregs per kilogram of body weight of the human subject, approximately 4.0×10⁷ or more Tregs per kilogram of body weight of the human subject, approximately 4.25×10⁷ or more Tregs per kilogram of body weight of the human subject, approximately 4.5×10⁷ or more Tregs per kilogram of body weight of the human subject, approximately 4.75×10⁷ or more Tregs per kilogram of body weight of the human subject, approximately 5.0×10⁷ or more Tregs per kilogram of body weight of the human subject, approximately 5.25×10⁷ or more Tregs per kilogram of body weight of the human subject, approximately 5.5×10⁷ or more Tregs per kilogram of body weight of the human subject, approximately 5.75×10⁷ or more Tregs per kilogram of body weight of the human subject, approximately 6.0×10⁷ or more Tregs per kilogram of body weight of the human subject, approximately 6.25×10⁷ or more Tregs per kilogram of body weight of the human subject, approximately 6.5×10⁷ or more Tregs per kilogram of body weight of the human subject, approximately 6.75×10⁷ or more Tregs per kilogram of body weight of the human subject, approximately 7.0×10⁷ or more Tregs per kilogram of body weight of the human subject, approximately 7.25×10⁷ or more Tregs per kilogram of body weight of the human subject, approximately 7.5×10⁷ or more Tregs per kilogram of body weight of the human subject, approximately 7.75×10⁷ or more Tregs per kilogram of body weight of the human subject, approximately 8.0×10⁷ or more Tregs per kilogram of body weight of the human subject, approximately 8.25×10⁷ or more Tregs per kilogram of body weight of the human subject, approximately 8.5×10⁷ or more Tregs per kilogram of body weight of the human subject, approximately 8.75×10⁷ or more Tregs per kilogram of body weight of the human subject, approximately 9.0×10⁷ or more Tregs per kilogram of body weight of the human subject, approximately 9.25×10⁷ or more Tregs per kilogram of body weight of the human subject, approximately 9.5×10⁷ or more Tregs per kilogram of body weight of the human subject, approximately 9.75×10⁷ or more Tregs per kilogram of body weight of the human subject, approximately 1.0×10⁸ or more Tregs per kilogram of body weight of the human subject. In some embodiments, the body weight of the human subject is actual body weight of the human subject. In other embodiments, the body weight of the human subject is ideal actual body weight of the human subject.

In some embodiments, the third population of CD45⁺ cells comprises Tcons. In other embodiments, the third population of CD45⁺ comprises at least one dose of Tcons. In some embodiments, the third population of CD45⁺ cells comprising Tcons, or comprising at least one dose of Tcons, comprises from approximately 1.0×10⁵ to approximately 1.0×10⁸ Tcons per kilogram of body weight of the human subject, from approximately 1.0×10⁵ to approximately 4.0×10⁷ Tcons per kilogram of body weight of the human subject, from approximately 1.0×10⁵ to approximately 1.0×10⁷ Tcons per kilogram of body weight of the human subject, or from approximately 5.0×10⁵ to approximately 5×10⁶ Tcons per kilogram of body weight of the human subject. In some embodiments, the third population of CD45⁺ cells comprising Tcons, or comprising at least one dose of Tcons, comprises approximately 1.0×10⁵ or more Tcons per kilogram of body weight of the human subject, approximately 1.5×10⁵ or more Tcons per kilogram of body weight of the human subject, approximately 2.0×10⁵ or more Tcons per kilogram of body weight of the human subject, approximately 2.5×10⁵ or more Tcons per kilogram of body weight of the human subject, approximately 3.0×10⁵ or more Tcons per kilogram of body weight of the human subject, approximately 3.5×10⁵ or more Tcons per kilogram of body weight of the human subject, approximately 4.0×10⁵ or more Tcons per kilogram of body weight of the human subject, approximately 4.5×10⁵ or more Tcons per kilogram of body weight of the human subject, approximately 5.0×10⁵ or more Tcons per kilogram of body weight of the human subject, approximately 5.5×10⁵ or more Tcons per kilogram of body weight of the human subject, approximately 6.0×10⁵ or more Tcons per kilogram of body weight of the human subject, approximately 6.5×10⁵ or more Tcons per kilogram of body weight of the human subject, approximately 7.0×10⁵ or more Tcons per kilogram of body weight of the human subject, approximately 7.5×10⁵ or more Tcons per kilogram of body weight of the human subject, approximately 8.0×10⁵ or more Tcons per kilogram of body weight of the human subject, approximately 8.5×10⁵ or more Tcons per kilogram of body weight of the human subject, approximately 9.0×10⁵ or more Tcons per kilogram of body weight of the human subject, approximately 9.5×10⁵ or more Tcons per kilogram of body weight of the human subject, approximately 1.0×10⁶ or more Tcons per kilogram of body weight of the human subject, approximately 1.25×10⁶ or more Tcons per kilogram of body weight of the human subject, approximately 1.5×10⁶ or more Tcons per kilogram of body weight of the human subject, approximately 1.75×10⁶ or more Tcons per kilogram of body weight of the human subject, approximately 2.0×10⁶ or more Tcons per kilogram of body weight of the human subject, approximately 2.25×10⁶ or more Tcons per kilogram of body weight of the human subject, approximately 2.5×10⁶ or more Tcons per kilogram of body weight of the human subject, approximately 2.75×10⁶ or more Tcons per kilogram of body weight of the human subject, approximately 3.0×10⁶ or more Tcons per kilogram of body weight of the human subject, approximately 3.25×10⁶ or more Tcons per kilogram of body weight of the human subject, approximately 3.5×10⁶ or more Tcons per kilogram of body weight of the human subject, approximately 3.75×10⁶ or more Tcons per kilogram of body weight of the human subject, approximately 4.0×10⁶ or more Tcons per kilogram of body weight of the human subject, approximately 4.25×10⁶ or more Tcons per kilogram of body weight of the human subject, approximately 4.5×10⁶ or more Tcons per kilogram of body weight of the human subject, approximately 4.75×10⁶ or more Tcons per kilogram of body weight of the human subject, approximately 5.0×10⁶ or more Tcons per kilogram of body weight of the human subject, approximately 5.25×10⁶ or more Tcons per kilogram of body weight of the human subject, approximately 5.5×10⁶ or more Tcons per kilogram of body weight of the human subject, approximately 5.75×10⁶ or more Tcons per kilogram of body weight of the human subject, approximately 6.0×10⁶ or more Tcons per kilogram of body weight of the human subject, approximately 6.25×10⁶ or more Tcons per kilogram of body weight of the human subject, approximately 6.5×10⁶ or more Tcons per kilogram of body weight of the human subject, approximately 6.75×10⁶ or more Tcons per kilogram of body weight of the human subject, approximately 7.0×10⁶ or more Tcons per kilogram of body weight of the human subject, approximately 7.25×10⁶ or more Tcons per kilogram of body weight of the human subject, approximately 7.5×10⁶ or more Tcons per kilogram of body weight of the human subject, approximately 7.75×10⁶ or more Tcons per kilogram of body weight of the human subject, approximately 8.0×10⁶ or more Tcons per kilogram of body weight of the human subject, approximately 8.25×10⁶ or more Tcons per kilogram of body weight of the human subject, approximately 8.5×10⁶ or more Tcons per kilogram of body weight of the human subject, approximately 8.75×10⁶ or more Tcons per kilogram of body weight of the human subject, approximately 9.0×10⁶ or more Tcons per kilogram of body weight of the human subject, approximately 9.25×10⁶ or more Tcons per kilogram of body weight of the human subject, approximately 9.5×10⁶ or more Tcons per kilogram of body weight of the human subject, approximately 9.75×10⁶ or more Tcons per kilogram of body weight of the human subject, approximately 1.0×10⁷ or more Tcons per kilogram of body weight of the human subject, approximately 1.25×10⁷ or more Tcons per kilogram of body weight of the human subject, approximately 1.5×10⁷ or more Tcons per kilogram of body weight of the human subject, approximately 1.75×10⁷ or more Tcons per kilogram of body weight of the human subject, approximately 2.0×10⁷ or more Tcons per kilogram of body weight of the human subject, approximately 2.25×10⁷ or more Tcons per kilogram of body weight of the human subject, approximately 2.5×10⁷ or more Tcons per kilogram of body weight of the human subject, approximately 2.75×10⁷ or more Tcons per kilogram of body weight of the human subject, approximately 3.0×10⁷ or more Tcons per kilogram of body weight of the human subject, approximately 3.25×10⁷ or more Tcons per kilogram of body weight of the human subject, approximately 3.5×10⁷ or more Tcons per kilogram of body weight of the human subject, approximately 3.75×10⁷ or more Tcons per kilogram of body weight of the human subject, approximately 4.0×10⁷ or more Tcons per kilogram of body weight of the human subject, approximately 4.25×10⁷ or more Tcons per kilogram of body weight of the human subject, approximately 4.5×10⁷ or more Tcons per kilogram of body weight of the human subject, approximately 4.75×10⁷ or more Tcons per kilogram of body weight of the human subject, approximately 5.0×10⁷ or more Tcons per kilogram of body weight of the human subject, approximately 5.25×10⁷ or more Tcons per kilogram of body weight of the human subject, approximately 5.5×10⁷ or more Tcons per kilogram of body weight of the human subject, approximately 5.75×10⁷ or more Tcons per kilogram of body weight of the human subject, approximately 6.0×10⁷ or more Tcons per kilogram of body weight of the human subject, approximately 6.25×10⁷ or more Tcons per kilogram of body weight of the human subject, approximately 6.5×10⁷ or more Tcons per kilogram of body weight of the human subject, approximately 6.75×10⁷ or more Tcons per kilogram of body weight of the human subject, approximately 7.0×10⁷ or more Tcons per kilogram of body weight of the human subject, approximately 7.25×10⁷ or more Tcons per kilogram of body weight of the human subject, approximately 7.5×10⁷ or more Tcons per kilogram of body weight of the human subject, approximately 7.75×10⁷ or more Tcons per kilogram of body weight of the human subject, approximately 8.0×10⁷ or more Tcons per kilogram of body weight of the human subject, approximately 8.25×10⁷ or more Tcons per kilogram of body weight of the human subject, approximately 8.5×10⁷ or more Tcons per kilogram of body weight of the human subject, approximately 8.75×10⁷ or more Tcons per kilogram of body weight of the human subject, approximately 9.0×10⁷ or more Tcons per kilogram of body weight of the human subject, approximately 9.25×10⁷ or more Tcons per kilogram of body weight of the human subject, approximately 9.5×10⁷ or more Tcons per kilogram of body weight of the human subject, approximately 9.75×10⁷ or more Tcons per kilogram of body weight of the human subject, approximately 1.0×10⁸ or more Tcons per kilogram of body weight of the human subject. In some embodiments, the body weight of the human subject is actual body weight of the human subject. In other embodiments, the body weight of the human subject is ideal actual body weight of the human subject.

In embodiments, the third population of CD45⁺ cells comprises at least about 0.1% CD34⁺ cells or from approximately 0.2% to approximately 20% CD34⁺ cells and/or at least about 0.1% Tregs. In various embodiments, the third population of CD45⁺ cells comprises a population of memory T cells (Tmems), e.g., Tmems that are CD3⁺ CD45RA⁻ CD45RO⁺. In some embodiments, the population of Tmems comprises more than about 3×10⁵ Tmems per kilogram of ideal body actual or ideal body weight of the human subject. In embodiments, the population of Tmems comprises from approximately 3×10⁵ to approximately 1×10⁹ Tmems per kilogram of ideal body actual or ideal body weight of the human subject. In various embodiments, the third population of CD45⁺ cells comprises a population of invariant natural killer T cells (iNKTs), e.g., iNKTs that are CD3⁺Vα24Jα18⁺. In some embodiments, the population of iNKTs comprises more than about 5×10² iNKTs per kilogram of ideal body actual or ideal body weight of the human subject. In embodiments, the population of iNKTs comprises from approximately 5×10² to approximately 1×10⁷ iNKTs per kilogram of ideal body actual or ideal body weight of the human subject. In some embodiments, the third population of CD45⁺ cells is co-cultured with donor cancer antigens/peptides and/or antigen-presenting cells.

In some embodiments, a cell population of the present disclosure, for example the first population of CD45⁺ cells, the second population of CD45⁺ cells, and/or the third population of CD45⁺ cells, may comprise one or more cells comprising one or more chimeric receptors. In some embodiments, the one or more cells may be T cells, NK cells, CTLs, Tregs, and/or NKT cells. In some embodiments, the one or more chimeric receptors comprise one or more extracellular antigen-binding domains that bind to one or more cancer-associated antigens. Any suitable cancer-associated antigen known in the art may be targeted. In some embodiments, the chimeric receptor is a chimeric antigen receptor or a T cell receptor.

Provided herein are compositions, multi-component pharmaceutical treatments, multi-component cellular therapy products, cell populations, solutions, formulations, kits, and/or methods for improved hematopoietic stem cell transplantation (HCT), for example, allogeneic hematopoietic stem cell transplantation (alloHSCT). Compositions, multi-component pharmaceutical treatments, multi-component cellular therapy products, cell populations, solutions, formulations, kits, and/or methods disclosed herein can comprise one or more cell populations that can be administered in combination with a GVHD prophylactic agent to achieve positive clinical outcomes. A cell population can comprise one or more types of cells, for example, hematopoietic stem and progenitor cells (HSPCs), conventional T cells (Tcons), regulatory T cells (Tregs), invariant natural killer T cells (iNKTs), memory T cells (Tmems), and combinations thereof.

The disclosure provides parameters for cell populations and methods of administering cell populations that can contribute to successful clinical outcomes in HCT recipient subjects. Without wishing to be bound by theory, parameters that can contribute to successful clinical outcomes in HCT recipient subjects include, for example, co-administration of a GVHD prophylactic agent as described herein (e.g., tacrolimus), populations administered, order and timing for the administration of different populations, purity standards for populations, methods for obtaining populations, methods of handling or storing populations, dosages of populations administered, methods for obtaining populations, and combinations thereof.

In various embodiments, administering comprises infusing into the human subject the first population of CD45⁺ cells, the population of cells enriched for Tregs (e.g., the second population of CD45⁺ cells), and the third population of CD45⁺ cells.

In some embodiments, the population of cells enriched for Tregs or the second population of CD45⁺ cells (as disclosed herein) is administered from approximately 5 minutes to approximately 5 hours after administration of the first population of CD45⁺ cells (as disclosed herein). In some embodiments, the population of cells enriched for Tregs or the second population of CD45⁺ cells is administered approximately 5 minutes, approximately 6 minutes, approximately 7 minutes, approximately 8 minutes, approximately 9 minutes, approximately 10 minutes, approximately 11 minutes, approximately 12 minutes, approximately 13 minutes, approximately 14 minutes, approximately 15 minutes, approximately 16 minutes, approximately 17 minutes, approximately 18 minutes, approximately 19 minutes, approximately 20 minutes, approximately 21 minutes, approximately 22 minutes, approximately 23 minutes, approximately 24 minutes, approximately 25 minutes, approximately 26 minutes, approximately 27 minutes, approximately 28 minutes, approximately 29 minutes, approximately 30 minutes, approximately 31 minutes, approximately 32 minutes, approximately 33 minutes, approximately 34 minutes, approximately 35 minutes, approximately 36 minutes, approximately 37 minutes, approximately 38 minutes, approximately 39 minutes, approximately 40 minutes, approximately 41 minutes, approximately 42 minutes, approximately 43 minutes, approximately 44 minutes, approximately 45 minutes, approximately 46 minutes, approximately 47 minutes, approximately 48 minutes, approximately 49 minutes, approximately 50 minutes, approximately 51 minutes, approximately 52 minutes, approximately 53 minutes, approximately 54 minutes, approximately 55 minutes, approximately 56 minutes, approximately 57 minutes, approximately 58 minutes, approximately 59 minutes, approximately 1 hour, approximately 1.10 hours, approximately 1.20 hours, approximately 1.30 hours, approximately 1.40 hours, approximately 1.50 hours, approximately 1.60 hours, approximately 1.70 hours, approximately 1.80 hours, approximately 1.90 hours, approximately 2 hours, approximately 2.10 hours, approximately 2.20 hours, approximately 2.30 hours, approximately 2.40 hours, approximately 2.50 hours, approximately 2.60 hours, approximately 2.70 hours, approximately 2.80 hours, approximately 2.90 hours, approximately 3 hours, approximately 3.10 hours, approximately 3.20 hours, approximately 3.30 hours, approximately 3.40 hours, approximately 3.50 hours, approximately 3.60 hours, approximately 3.70 hours, approximately 3.80 hours, approximately 3.90 hours, approximately 4 hours, approximately 4.10 hours, approximately 4.20 hours, approximately 4.30 hours, approximately 4.40 hours, approximately 4.50 hours, approximately 4.60 hours, approximately 4.70 hours, approximately 4.80 hours, approximately 4.90 hours, or approximately 5 hours after administration of the first population of CD45⁺ cells.

In some embodiments, the third population of CD45⁺ cells (as disclosed herein) is administered at least about 12 hours after the first population of CD45⁺ cells (as disclosed herein), the third population of CD45⁺ cells is administered from approximately 24 to approximately 96 hours after the first population of CD45⁺ cells, the third population of CD45⁺ cells is administered from approximately 36 to approximately 60 hours after the first population of CD45⁺ cells, the third population of CD45⁺ cells is administered at least about 12 hours after the population of cells enriched for Tregs (as disclosed herein), the third population of CD45⁺ cells is administered from approximately 24 to approximately 96 hours after the population of cells enriched for Tregs, and/or the third population of CD45⁺ cells is administered from approximately 36 to approximately 60 hours after the population of cells enriched for Tregs or the second population of CD45⁺ cells.

In some embodiments, the third population of CD45⁺ cells (as disclosed herein) is administered from approximately 12 hours to approximately 120 hours after administration of the first population of isolated CD45⁺ cells (as disclosed herein). In some embodiments, the third population of CD45⁺ cells is administered approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately, 24 hours, approximately 25 hours, approximately 26 hours, approximately 27 hours, approximately 28 hours, approximately 29 hours, approximately 30 hours, approximately 31 hours, approximately 32 hours, approximately 33 hours, approximately, 34 hours, approximately 35 hours, approximately 36 hours, approximately 37 hours, approximately 38 hours, approximately 39 hours, approximately 40 hours, approximately 41 hours, approximately 42 hours, approximately 43 hours, approximately, 44 hours, approximately 45 hours, approximately 46 hours, approximately 47 hours, approximately 48 hours, approximately 49 hours, approximately 50 hours, approximately 51 hours, approximately 52 hours, approximately 53 hours, approximately, 54 hours, approximately 55 hours, approximately 56 hours, approximately 57 hours, approximately 58 hours, approximately 59 hours, approximately 60 hours, approximately 61 hours, approximately 62 hours, approximately 63 hours, approximately, 64 hours, approximately 65 hours, approximately 66 hours, approximately 67 hours, approximately 68 hours, approximately 69 hours, approximately 70 hours, approximately 71 hours, approximately 72 hours, approximately 73 hours, approximately, 74 hours, approximately 75 hours, approximately 76 hours, approximately 77 hours, approximately 78 hours, approximately 79 hours, approximately 80 hours, approximately 81 hours, approximately 82 hours, approximately 83 hours, approximately, 84 hours, approximately 85 hours, approximately 86 hours, approximately 87 hours, approximately 88 hours, approximately 89 hours, approximately 90 hours, approximately 91 hours, approximately 92 hours, approximately 93 hours, approximately, 94 hours, approximately 95 hours, approximately 96 hours, approximately 97 hours, approximately 98 hours, approximately 99 hours, approximately 100 hours, approximately 101 hours, approximately 102 hours, approximately 103 hours, approximately, 104 hours, approximately 105 hours, approximately 106 hours, approximately 107 hours, approximately 108 hours, approximately 109 hours, approximately 110 hours, approximately 111 hours, approximately 112 hours, approximately 113 hours, approximately, 114 hours, approximately 115 hours, approximately 116 hours, approximately 117 hours, approximately 118 hours, approximately 119 hours, or approximately 120 hours after administration of the first population of CD45⁺ cells.

In some embodiments, the third population of CD45⁺ cells (as disclosed herein) is administered from approximately 12 hours to approximately 120 hours after administration of the population of cells enriched for Tregs or the second population of CD45⁺ cells (as disclosed herein). In some embodiments, the third population of CD45⁺ cells is administered approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately, 24 hours, approximately 25 hours, approximately 26 hours, approximately 27 hours, approximately 28 hours, approximately 29 hours, approximately 30 hours, approximately 31 hours, approximately 32 hours, approximately 33 hours, approximately, 34 hours, approximately 35 hours, approximately 36 hours, approximately 37 hours, approximately 38 hours, approximately 39 hours, approximately 40 hours, approximately 41 hours, approximately 42 hours, approximately 43 hours, approximately, 44 hours, approximately 45 hours, approximately 46 hours, approximately 47 hours, approximately 48 hours, approximately 49 hours, approximately 50 hours, approximately 51 hours, approximately 52 hours, approximately 53 hours, approximately, 54 hours, approximately 55 hours, approximately 56 hours, approximately 57 hours, approximately 58 hours, approximately 59 hours, approximately 60 hours, approximately 61 hours, approximately 62 hours, approximately 63 hours, approximately, 64 hours, approximately 65 hours, approximately 66 hours, approximately 67 hours, approximately 68 hours, approximately 69 hours, approximately 70 hours, approximately 71 hours, approximately 72 hours, approximately 73 hours, approximately, 74 hours, approximately 75 hours, approximately 76 hours, approximately 77 hours, approximately 78 hours, approximately 79 hours, approximately 80 hours, approximately 81 hours, approximately 82 hours, approximately 83 hours, approximately, 84 hours, approximately 85 hours, approximately 86 hours, approximately 87 hours, approximately 88 hours, approximately 89 hours, approximately 90 hours, approximately 91 hours, approximately 92 hours, approximately 93 hours, approximately, 94 hours, approximately 95 hours, approximately 96 hours, approximately 97 hours, approximately 98 hours, approximately 99 hours, approximately 100 hours, approximately 101 hours, approximately 102 hours, approximately 103 hours, approximately, 104 hours, approximately 105 hours, approximately 106 hours, approximately 107 hours, approximately 108 hours, approximately 109 hours, approximately 110 hours, approximately 111 hours, approximately 112 hours, approximately 113 hours, approximately, 114 hours, approximately 115 hours, approximately 116 hours, approximately 117 hours, approximately 118 hours, approximately 119 hours, or approximately 120 hours after administration of the population of cells enriched for Tregs or the second population of CD45⁺ cells.

HSPCs can have extensive self-renewal capacity, and an ability to differentiate into specialized cell types, for example, an ability to reconstitute all hematopoietic cell lineages. HSPCs can undergo asynchronous replication, where two daughter cells are produced with different phenotypes. HSPCs cells can exist in a mitotically quiescent form. HSPCs can be derived from bone marrow, peripheral blood, and/or umbilical cord blood.

Subsets of immune cells, such as conventional T cells (Tcons), regulatory T cells (Tregs), invariant natural killer T cells (iNKTs), and memory T cells (Tmems) can contribute to aspects of GVHD following HCT, and can also contribute to, for example, GVT immune responses, immune reconstitution, infection susceptibility, and patient survival.

GVHD can be mediated in large part by donor T cells, which can elicit inflammatory responses upon recognition of recipient antigens. T cell depletion (TCD) of cell populations for transplantation to a subject can be undertaken to decrease the likelihood of acute and/or chronic GVHD. T cells can be depleted using methods including, but not limited to, physical adsorption of T cells to protein ligands such as lectins, immunodepletion with T cell specific antibodies, and immunoaffinity techniques (for example, use of T cell or lymphocyte-specific antibodies in immunoadsorption columns, magnetic activated cell sorting (MACS), or fluorescent activated cell sorting (FACS)). Applying TCD techniques to donor grafts can result in, for example, 10-fold to 105-fold depletion of T cells, and reduced incidence of GVHD. However, TCD can also result in increased incidence of cancer relapse, as the lack of T cells can reduce a graft-versus-tumor (GVT) immune response. Additionally, TCD can result in impaired immune recovery, and increased susceptibility to infections.

Both GVT and GVHD can be largely mediated by conventional T cells (Tcons), which mount immune responses upon recognition of cognate antigen by T cell receptors (tumor antigens for GVT, non-tumor recipient antigens for GVHD). Tcons can, for example, contribute to GVT, GVHD, or a combination thereof. In some embodiments, administration of Tcons after administration of Tregs can enhance GVT immunity, and/or reduce susceptibility to infection.

Tcons can broadly refer to all CD3⁺ T cells, cells expressing CD3 and CD4 or cells expressing CD3 and CD8, cells expressing medium to high levels of CD127, cells expressing CD3 and medium to high levels of CD127, cells expressing CD3, cells expressing medium to high levels of CD127, and cells expressing CD4 or CD8. In some embodiments, Tcons do not express Vα24Jα18 TCR. Tcons and Regulatory T cells (“Tregs”) can be non-mutually-exclusive cell populations. In some embodiments, Tcons and Tregs are mutually exclusive cell populations.

Regulatory T cells (“Tregs”) are a specialized subpopulation of T cells that negatively regulate (e.g., suppress) activation of the immune system and thereby promote immune tolerance. Without wishing to be bound by theory, cell populations of the disclosure enriched for Tregs contribute to positive clinical outcomes by, for example, reducing the incidence and/or severity of GVHD in a transplant recipient subject, and/or improving immune reconstitution in a transplant recipient. Administering cell population enriched for Tregs with a population of CD45⁺ cells that comprises, at least, HSPCs can, for example, facilitate retention of graft versus tumor (GVT) and reduced incidence and/or severity of GVHD. Without wishing to be bound by theory, administering population of cells enriched for Tregs can prevent GVHD, and administering third population of CD45⁺ cells that comprises, at least, Tcons can promote GVT effects, for example, relative to alternate hematopoietic stem cell transplantation (HCT) methods, i.e., methods that are distinct from the compositions, multi-component pharmaceutical treatments, multi-component cellular therapy products, cell populations, solutions, formulations, kits, and/or methods disclosed herein. In some embodiments, administering a population of cells enriched for Tregs reduces the risk of developing GVHD, and administering third population of CD45⁺ cells that comprises, at least, Tcons promotes GVT effects relative to alternate HCT methods, i.e., methods that are distinct from the compositions, multi-component pharmaceutical treatments, multi-component cellular therapy products, cell populations, solutions, formulations, kits, and/or methods disclosed herein.

As used herein, an alternate composition lacks one or more cell populations and/or prophylactic agent that are disclosed herein and/or recited in the claims. As examples, an alternate composition lacks one or more of a cell population comprising HSPCs, a cell population comprising Tregs, a cell population comprising Tcons, and a prophylactic agent. In some embodiments, an alternate composition or treatment regimen comprises an additional cell population or agent compared to a composition or treatment regimen of the disclosure, e.g., a an additional or different GVHD prophylactic agent.

There are a number of subsets of Tregs, for example, TCRαβ⁺CD4⁺ regulatory T cells, which include natural regulatory T cells (nTregs) and induced regulatory T cells (iTregs). nTregs can be T cells produced in the thymus and delivered to the periphery as a long-lived lineage of self-antigen-specific lymphocytes. iTregs can be recruited from circulating lymphocytes and acquire regulatory properties under particular conditions of stimulation in the periphery. nTregs and iTregs are CD4⁺CD25⁺; both can inhibit proliferation of CD4⁺CD25− T cells in a dose-dependent manner. In some embodiments, Tregs are anergic and do not proliferate upon TCR stimulation. In addition to being positive for CD4 and CD25, Tregs can be positive for the transcription factor FOXP3, an intracellular marker. Tregs can be identified or selected based on various marker expression profiles. Non-limiting examples of marker expression profiles that can be used to select Tregs include (1) CD4⁺ CD25⁺CD127^{dim}, (2) CD4⁺FOXP3⁺, (3) CD3⁺CD4⁺CD25⁺, (4) CD3⁺CD4⁺CD25⁺CD127^{dim}, (5) CD3⁺CD4⁺CD25⁺CD127^{dim}FOXP3⁺, (6) CD3⁺FOXP3⁺, (7) CD3⁺CD4⁺FOXP3⁺, (8) CD3⁺ CD4⁺CD25⁺FOXP3⁺, (9) CD3⁺CD25⁺FOXP3⁺, (10) CD3⁺CD25⁺CD127^{dim}, (11) CD4⁺CD25⁺, (12) CD4⁺CD25⁺CD127^{dim}FOXP3⁺, (13) FOXP3⁺, (14) CD4⁺FOXP3⁺, (15) CD4⁺CD25⁺FOXP3⁺, (16) CD25⁺FOXP3⁺, and (17) CD25⁺ CD127^{dim}.

Selection based on certain expression profiles can be achieved based on extracellular markers and without requiring cell permeabilization, for example, selection based on CD4⁺CD25⁺CD127^{dim}.

A cell population that comprises Tregs can, for example, reduce the incidence of graft rejection, reduce the incidence and/or severity of GVHD, promote hematopoietic reconstitution, promote immune reconstitution, promote mixed chimerism, or a combination thereof.

A cell population of the disclosure can comprise invariant natural killer T cells (iNKTs). iNKTs are subclass of CD1d-restricted Natural Killer T (NKT) cells that express a highly conserved αβ-T cell receptor that comprises of Vα24Jα18 TCRα chain in humans (referred to herein as “Vα24Jα18⁺”). iNKT cells can be identified by binding with CD1d-multimers like that are loaded with a-galactosylceramide (GalCer), PBS-57, PBS-44 or other natural or synthetic glycolipids. Another method of identification is an antibody or combination of antibodies that specifically recognize the Vα24Jα18 region. Examples include a Vα24 antibody, a Jα18 antibody, or the monoclonal antibody clone 6B11 which binds specifically to a unique region of the Vα24Jα18 TCR and can be used to identify iNKT cells. iNKTs can be CD3⁺Vα24Jα18⁺.

In some embodiments, iNKTs can promote engraftment, promote GVT, reduce incidence and/or severity of GVHD, decrease susceptibility to cancer relapse, decrease susceptibility to infection, or a combination thereof. In some embodiments, iNKTs promote the activity of Tregs. In some embodiments, iNKTs promote the activity of HSPCs.

A cell population of the disclosure can comprise memory T cells (Tmems). Tmems can refer to antigen-experienced T cells that express, for example, the phenotypic markers CD45RO, TCRα, TCRβ, CD3, CD4, CD95, and IL-2Rβ or the phenotypic markers CD45RO, TCRα, TCRβ, CD3, CD8, CD95, and IL-2Rβ. Tmems provide immunity and are capable of persisting for a long period of time in an inactive state. Tmems are able to rapidly acquire effector functions upon re-challenge with antigen. A population of Tmems can include any combination of the subclasses T central memory cells and T effector memory cells. In some embodiments, Tmems are CD3⁺ CD45RA CD45RO⁺. In various methods, Tmems administered to a subject receiving HCT can, for example, promote GVT, reduce GVHD, decrease susceptibility to cancer relapse, decrease susceptibility to infection, or a combination thereof.

### Acquisition, Processing, and Preparation of Cells

Certain aspects of the present disclosure relate to methods for preparing the compositions, multi-component pharmaceutical treatments, multi-component cellular therapy products, cell populations, solutions, formulations, and/or kits of the present disclosure.

In some embodiments, at least one mobilized peripheral blood donation is collected from a donor or at most two mobilized peripheral blood donations are collected from the donor. In some embodiments, the mobilized peripheral blood donation is an HSPC-mobilized peripheral blood apheresis donation. In some embodiments, prior to peripheral blood donation, the donor may be vaccinated with a tumor antigen and/or a pathogen to enhance graft versus infection.

In embodiments, at least one of the mobilized peripheral blood donations is processed and sorted to enrich CD34⁺ cells and Tregs. In some embodiments, the peripheral blood donation is further processed and sorted or alternatively processed and sorted to enrich CD3⁺ cells (e.g., Tcons). In embodiments, at least one of the mobilized peripheral blood donations is processed and sorted to enrich CD34⁺ cells, Tregs, and/or Tcons. The processing and sorting for the CD34⁺ cells, Tregs, and Tcons maybe done in any odder. For example, at least one of the mobilized peripheral blood donations may be processed and sorted to first enrich CD34⁺ cells, then Tcons, and then Tregs. Alternatively, at least one of the mobilized peripheral blood donations may be processed and sorted to first enrich CD34⁺ cells, then Tregs, and then Tcons. Alternatively, at least one of the mobilized peripheral blood donations may be processed and sorted to first enrich Tregs, then CD34⁺ cells, and then Tcons. Alternatively, at least one of the mobilized peripheral blood donations may be processed and sorted to first enrich Tregs, then Tcons, and then CD34⁺ cells. Alternatively, at least one of the mobilized peripheral blood donations may be processed and sorted to first enrich Tcons, then CD34⁺ cells, and then Tregs. Alternatively, at least one of the mobilized peripheral blood donations may be processed and sorted to first enrich Tcons, then Tregs, and then CD34⁺ cells. In some embodiments, at least one of the mobilized peripheral blood donations may be processed and sorted to first enrich Tregs and then CD34⁺ cells, without enrichment for Tcons. In other embodiments, at least one of the mobilized peripheral blood donations may be processed and sorted to first enrich CD34⁺ cells and then Tregs without enrichment for Tcons.

In some embodiments, the processing and sorting time of the one or more of the mobilized peripheral blood donations is less than about 40 hours, the processing and sorting time of the one or more of the mobilized peripheral blood donations is less than about 35 hours, the processing and sorting time of the one or more of the mobilized peripheral blood donations is less than about 30 hours, the processing and sorting time of the one or more of the mobilized peripheral blood donations is less than about 25 hours, the processing and sorting time of the one or more of the mobilized peripheral blood donations is less than about 20 hours, the processing and sorting time of the one or more of the mobilized peripheral blood donations is less than about 15 hours, and/or the processing and sorting time of the one or more of the mobilized peripheral blood donations is at most about 35 hours, the processing and sorting time of the one or more of the mobilized peripheral blood donations is at most about 30 hours, the processing and sorting time of the one or more of the mobilized peripheral blood donations is at most about 25 hours, the processing and sorting time of the one or more of the mobilized peripheral blood donations is at most about 20 hours, or the processing and sorting time of the one or more of the mobilized peripheral blood donations is at most about 15 hours.

In various embodiments, the one or more of the mobilized peripheral blood donations is processed and sorted using one or more immune-separation particles (ISPs), e.g., ISPs comprise affinity reagents such as immuno-magnetic separation particles which may be antibodies each conjugated to an iron-containing particle, and/or immuno-fluorescent separation particles which may be antibodies each conjugated to a fluorophore or other fluorescent particle.

In some embodiments, the affinity reagents comprise a plurality of CD34-reagents (e.g., an anti-CD34 antibody) that binds to one or more CD34 receptors on a HSPC. In some cases, at least a portion of the plurality of ISPs are attached to CD34⁺receptors on the HPSC's of the HSPC cell population; optionally, an average number of ISPs per HSPC in the HSPC cell population is less than about 20,000, an average number of ISPs per HSPC in the HSPC cell population is equal to or less than about 10,000, and/or an average number of ISPs per HSPC in the HSPC cell population is from approximately 1000 to approximately 20,000.

In some embodiments, an average number of ISPs per HSPC in the HSPC cell population may be about 1,500 to approximately 20,000. In some embodiments, an average number of ISPs per HSPC in the HSPC cell population may be at least about 1,500. In some embodiments, an average number of ISPs per HSPC in the HSPC cell population may be at most about 20,000. In some embodiments, an average number of ISPs per HSPC in the HSPC cell population may be about 1,500 to approximately 2,000, about 1,500 to approximately 5,000, about 1,500 to approximately 6,000, about 1,500 to approximately 10,000, about 1,500 to approximately 12,000, about 1,500 to approximately 15,000, about 1,500 to approximately 20,000, about 2,000 to approximately 5,000, about 2,000 to approximately 6,000, about 2,000 to approximately 10,000, about 2,000 to approximately 12,000, about 2,000 to approximately 15,000, about 2,000 to approximately 20,000, about 5,000 to approximately 6,000, about 5,000 to approximately 10,000, about 5,000 to approximately 12,000, about 5,000 to approximately 15,000, about 5,000 to approximately 20,000, about 6,000 to approximately 10,000, about 6,000 to approximately 12,000, about 6,000 to approximately 15,000, about 6,000 to approximately 20,000, about 10,000 to approximately 12,000, about 10,000 to approximately 15,000, about 10,000 to approximately 20,000, about 12,000 to approximately 15,000, about 12,000 to approximately 20,000, or about 15,000 to approximately 20,000. In some embodiments, an average number of ISPs per HSPC in the HSPC cell population may be about 1,500, about 2,000, about 5,000, about 6,000, about 10,000, about 12,000, about 15,000, or about 20,000. In some embodiments, an average number of ISPs per HSPC in the HSPC cell population may be at least 1,500, 2,000, 5,000, 6,000, 10,000, 12,000, 15,000, or 20,000. In some embodiments, an average number of ISPs per HSPC in the HSPC cell population may be at most 1,500, 2,000, 5,000, 6,000, 10,000, 12,000, 15,000, or 20,000.

In some embodiments, the affinity reagents comprise a plurality of CD25-reagents (e.g., an anti-CD25 antibody) that binds to one or more CD25 receptors on a cell. In some cases, at least a portion of the plurality of ISPs are attached to CD25⁺receptors on the cells of the Treg cell population; optionally, an average number of ISPs per T-reg cell in the Treg population is equal or less than about 4000 or an average number of ISPs per T-reg cell in the Treg population is from approximately 1500 to approximately 2500. In some cases, at least a portion of the plurality of ISPs are attached to CD3⁺receptors on the cells of the heterogenous cell population; optionally, an average number of ISPs per cell in population of T heterogenous is less than about 4,000. In some embodiments, an average number of ISPs per Tcon cell in the Tcon cell population may be about 100 to approximately 1,000. In some embodiments, an average number of ISPs per Tcon cell in the Tcon cell population may be at least about 100. In some embodiments, an average number of ISPs per Tcon cell in the Tcon cell population may be at most about 1,000. In some embodiments, an average number of ISPs per Tcon cell in the Tcon cell population may be about 100 to approximately 200, about 100 to approximately 500, about 100 to approximately 1,000, about 200 to approximately 500, about 200 to approximately 1,000, or about 500 to approximately 1,000. In some embodiments, an average number of ISPs per Tcon cell in the Tcon cell population may be about 100, about 200, about 500, or about 1,000. In some embodiments, an average number of ISPs per Tcon cell in the Tcon cell population may be at least 100, 200, 500, or 1,000. In some embodiments, an average number of ISPs per Tcon cell in the Tcon cell population may be at most 100, 200, 500, or 1,000.

In some embodiments, an average number of ISPs per Treg cells in the Treg cell population may be about 500 to approximately 4,000. In some embodiments, an average number of ISPs per Treg cells in the Treg cell population may be at least about 500. In some embodiments, an average number of ISPs per Treg cells in the Treg cell population may be at most about 4,000. In some embodiments, an average number of ISPs per Treg cells in the Treg cell population may be about 500 to approximately 1,000, about 500 to approximately 1,500, about 500 to approximately 2,000, about 500 to approximately 2,500, about 500 to approximately 3,000, about 500 to approximately 4,000, about 1,000 to approximately 1,500, about 1,000 to approximately 2,000, about 1,000 to approximately 2,500, about 1,000 to approximately 3,000, about 1,000 to approximately 4,000, about 1,500 to approximately 2,000, about 1,500 to approximately 2,500, about 1,500 to approximately 3,000, about 1,500 to approximately 4,000, about 2,000 to approximately 2,500, about 2,000 to approximately 3,000, about 2,000 to approximately 4,000, about 2,500 to approximately 3,000, about 2,500 to approximately 4,000, or about 3,000 to approximately 4,000. In some embodiments, an average number of ISPs per Treg cells in the Treg cell population may be about 500, about 1,000, about 1,500, about 2,000, about 2,500, about 3,000, or about 4,000. In some embodiments, an average number of ISPs per Treg cells in the Treg cell population may be at least 500, 1,000, 1,500, 2,000, 2,500, 3,000, or 4,000. In some embodiments, an average number of ISPs per Treg cells in the Treg cell population may be at most 500, 1,000, 1,500, 2,000, 2,500, 3,000, or 4,000.

In some embodiments, CD25-enriched cell populations are further processed by sorting with one or more ISPs comprising immuno-fluorescent separation particles (e.g., antibodies conjugated to fluorophores) specific for CD127 and CD4. In some embodiments, a CD25-enriched Treg population of the present disclosure is sorted using the CD127-specific ISPs and CD4− specific ISPs to obtain a CD25⁺CD4⁺CD127^{dim} Treg population. In some embodiments, the Treg population is also FOXP3⁺. In some embodiments, a CD127^{dim} cell population may include a CD127⁻ population and/or a CD127^{low} population.

In some embodiments, the affinity reagents comprise a plurality of CD3-reagents (e.g., an anti-CD3 antibody) that binds to one or more CD3 receptors on a cell (e.g., a Tcon). In some cases, at least a portion of the plurality of ISPs are attached to CD3⁺ receptors on the cells of the Tcon cell population; optionally, an average number of ISPs per T cell in the Tcon population is equal or less than about 4000 or an average number of ISPs per T cell in the Tcon population is from approximately 1500 to approximately 2500. In some cases, at least a portion of the plurality of ISPs are attached to CD3⁺ receptors on the cells of the heterogenous cell population; optionally, an average number of ISPs per cell in population of T heterogenous is less than about 4,000.

In various embodiments, cells of the mobilized peripheral blood donation are sorted such that the first population of CD45⁺ cells comprises at most about 10% granulocytes. In some cases, cells of the mobilized peripheral blood donation are sorted such that the first population of CD45⁺ cells comprises at most about 7% granulocytes.

In some embodiments, cells of the mobilized donor peripheral blood donation are sorted such that the first population of CD45⁺ cells comprises at most about 4% monocytes. In some cases, cells of the mobilized donor peripheral blood donation are sorted such that the first population of CD45⁺ cells comprises at least about 0.1% monocytes.

In embodiments, cells of the mobilized donor peripheral blood donation are sorted such that the population enriched for Tregs comprises at most about 10% CD25− cells.

In some embodiments, a cell population of the disclosure is obtained from whole blood. A cell population of the disclosure can be obtained from a peripheral blood apheresis product, for example, a mobilized peripheral blood apheresis product, e.g., mobilized by administration of GCSF, GM-CSF, MOZOBIL® (plerixafor), and combinations thereof, to a donor. A cell population of the disclosure can be obtained from at least one apheresis product, two apheresis products, three apheresis products, four apheresis products, five apheresis products, six apheresis products, or more. In some embodiments, a cell population of the disclosure is obtained from one apheresis product. In some embodiments, a cell population of the disclosure is obtained from two apheresis products. In some embodiments, a cell population of the disclosure is obtained from an apheresis product from one donor and an apheresis product from an at least second donor.

In some embodiments, a cell population of the disclosure is obtained from bone marrow sample. In some embodiments, a cell population of the disclosure can be obtained from a bone marrow ample, for example, a mobilized bone marrow sample, e.g., mobilized by administration of GCSF, GM-CSF, MOZOBIL® (plerixafor), and combinations thereof, to a donor.

In some embodiments, a cell population of the disclosure is obtained from umbilical cord blood.

A cell population of the disclosure can be refined by selection from a population of cells, for example, peripheral blood or a peripheral blood apheresis product. Selection methods for cell populations can comprise methods involving positive or negative selection of a cell population of interest. Selection methods for cell populations can comprise affinity reagents, including but not limited to an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, or a polyaminoacid. In some embodiments, the affinity reagent is directly conjugated to a detection reagent and/or purification reagent. In some cases, the detection reagent and purification reagent are the same. In some cases, the detection reagent and purification reagent are different. For example, the detection reagent and/or purification reagent is fluorescent, magnetic, or the like. In some cases, the detection reagent and/or purification reagent is a magnetic particle for column purification. For example, magnetic column purification may be performed using the Miltenyi system (CliniMACs) of columns, antibodies, buffers, preparation materials and reagents.

In various embodiments, at least one of the cell populations have a plurality of immuno-separation particles (ISPs) attached to receptors on the cells of the cell population. In some cases, the plurality of ISPs are immuno-magnetic separation particles. In some embodiments, the plurality of ISPs comprise an antibody conjugated to an iron containing particle. In some cases, at least a portion of the plurality of ISPs are attached to CD34⁺ receptors on the HPSC's of the HSPC cell population; optionally, an average number of ISPs per HSPC in the HSPC cell population is less than about 6,000, an average number of ISPs per HSPC in the HSPC cell population is equal to or less than about 3,000, and/or an average number of ISPs per HSPC in the HSPC cell population is from approximately 1700 to approximately 3,000. In some cases, at least a portion of the plurality of ISPs are attached to CD25⁺ receptors on the cells of the Treg cell population; optionally, an average number of ISPs per T-reg cell in the Treg population is equal or less than about 1700 or an average number of ISPs per T-reg cell in the Treg population is from approximately 1400 to approximately 1700. In some cases, at least a portion of the plurality of ISPs are attached to CD3⁺ receptors on the cells of the heterogenous cell population; optionally, an average number of ISPs per cell in population of T heterogenous is less than about 1,000.

Affinity reagents can comprise immunoaffinity reagents, utilizing the binding specificity of antibodies or fragments or derivatives thereof to positively or negatively select for a cell population of interest. Selection methods for cell populations can comprise an affinity agent and a column, such as magnetic activated cell sorting (MACS) with specific antibodies and microbeads. Selection methods for cell populations can comprise fluorescent activated cell sorting (FACS), with cell populations sorted based on staining profiles with one or more fluorescently-conjugated antibodies. Selection methods for cell populations can comprise physical adsorption, for example, physical adsorption of T cells to protein ligands such as lectins.

HSPCs can be obtained by harvesting from bone marrow or from peripheral blood. Bone marrow can be aspirated from the posterior iliac crest or the anterior iliac crest while the donor is under either local or general anesthesia. HSPCs can be obtained by harvesting from peripheral blood, for example, by peripheral blood apheresis. The number of stem cells harvested can be increased by treating the donor with a mobilization agent, i.e., an agent that mobilizes stem cells from the bone marrow into peripheral blood. Non-limiting examples of mobilization agents include granulocyte colony-stimulating factor (G-CSF), granulocyte macrophage colony-stimulating factor (GM-CSF), stem cell factor (SCF), a SDF-1 antagonist, a CXCR4 antagonist (e.g., POL6326, BKT-140, TG-0054, NOX-A12), MOZOBIL® (plerixafor), a CXCR2 ligand (e.g., GROβ), a sphingosine-1-phosphatase (S1P) agonist. (e.g., SEW2871), a VCAM/VLA-4 inhibitor (e.g., BIO5192), a proteosome inhibitor (e.g., Bortezomib), parathyroid hormone, a hypoxia inducible factor (HIF) stabilizer (e.g., FG-4497), and combinations thereof. Techniques to mobilize stem cells into peripheral blood can comprise administering to a donor, for example, 10 to 40 u/kg/day of a mobilization agent. A mobilization agent can be administered to the donor in, for example, 1, 2, 3, 4, 5, 6, 7, 8, 9, or 10 doses. An apheresis product can be isolated from a donor about, for example, 1, 2, 3, 4, 5, 6, 7, 8, 9, 10, 11, 12, 13, 14, 15, 16, 17, 18, 19, 20, 21, 22, 23, 24, 26, 28, or 30 hour(s) after a dose of mobilization agent.

A population of CD45⁺ cells of the disclosure can comprise a HSPCs. The HSPCs can be selected based on expression of CD34. For example, the HSPCs of the disclosure can be selected using anti-CD34 antibodies as part of a magnetic activated cell sorting (MACS) or fluorescent activated cell sorting (FACS) system.

The number of HPSCs in a population of CD45⁺ cells can be determined, for example, by quantifying CD34⁺ cells via flow cytometry. In some embodiments, dose calculations are adjusted based on measures of cell viability measurements, e.g., viability determined via flow cytometry with propidium iodide or 7-AAD, or via trypan blue exclusion.

A cell population of the disclosure can be enriched for Tregs (e.g., the second population of CD45⁺ cells). Tregs can be selected based on expression of markers including CD3, CD4, CD25, CD127, FOXP3, and combinations thereof.

Tregs can be selected using magnetic activated cell sorting (MACS). Tregs can be selected using fluorescent activated cell sorting (FACS). Tregs can be selected using multiple procedures, for example, multiple MACS selections, multiple FACS selections, or a combination of MACS and FACS selections. For example, a first selection may be performed for expression of CD25, isolating CD25⁺ cells from a hematopoietic cell sample, for example with MACS. A second selection may be performed by contacting the CD25⁺ cells with antibodies specific for CD4 and for CD127, where FACS is used to isolate cells that are CD4⁺CD127^{dim}.

Tregs can be isolated from whole blood. Tregs can be isolated from a peripheral blood apheresis product. Tregs can be isolated from a population of cells previously enriched and/or depleted for one or more other cell types, e.g., isolated from a population of cells depleted of CD34⁺ cells. In some embodiments, Tregs are isolated from the flow-through fraction of a CD34⁺ MACS selection.

The number of Tregs in a population of cells can be determined, for example, by flow cytometry, where Tregs can be identified as, for example, CD4⁺CD25⁺CD127^{dim} or CD4⁺ FOXP3⁺. Dose calculations can be adjusted based on measures of cell viability measurements, e.g., viability determined via flow cytometry with propidium iodide or 7-AAD, or via trypan blue exclusion.

In some embodiments, the third population of CD45⁺ cells can comprise a population of Tcons. The third population of CD45⁺ cells that comprises, at least, Tcons can be sourced from peripheral blood. The third population of CD45⁺ cells can be sourced from a peripheral blood apheresis product.

In some embodiments, no selection steps are carried out, and a population of CD45⁺ cells that comprises, at least, Tcons is sourced directly from an aliquot of peripheral blood or apheresis product. In some embodiments, a population of cells can be enriched for Tcons, for example, by sorting based on the expression of various markers using MACS, FACS, or a combination thereof. In some embodiments, a third population of CD45⁺ cells can be enriched by sorting for CD3⁺ cells. In some embodiments, a third population of CD45⁺ cells can be enriched by sorting for CD4⁺ and CD8⁺ cells. In some embodiments, a third population of CD45⁺ cells can be enriched by negative selection, where non-Tcon cells are removed, for example, by MACS depletion of cells expressing CD34, CD19, CD25, or a combination thereof.

The number of Tcons present in a third population of CD45⁺ cells can be quantified, for example, by quantifying CD3⁺ cells via flow cytometry. The number of CD3⁺ cells in an aliquot can be determined and a volume comprising an appropriate dose of CD3 cells administered to the recipient. Dose calculations can be adjusted based on measures of cell viability, e.g., viability determined via flow cytometry with propidium iodide or 7-AAD, or via trypan blue exclusion.

An apheresis product of the disclosure can be split into two portions, one portion used to provide the third population of CD45⁺ cells that comprises, at least, Tcons and the other portion to isolate and purify the population of CD45⁺ cells that comprises, at least, HSPCs, and the cell population enriched for Tregs. In alternate embodiments, CD34⁺ cells are isolated and purified from the apheresis product, creating a CD34-negative cell fraction from which the cell Treg are then isolated to help provide the cell population enriched for Tregs.

A cell population of the disclosure can comprise a population of iNKTs. A population of iNKTs can be sourced from peripheral blood. A population of iNKTs can be sourced from a peripheral blood apheresis product.

A population of cells can be enriched for iNKTs, for example, by sorting based on the expression of various markers using MACS, FACS, or a combination thereof. A population of iNKTs can be enriched, for example, by sorting for CD3 Vα24Jα18⁺ cells.

The number of iNKTs present in a population can be quantified, for example, by quantifying CD3 Vα24Jα18⁺ cells via flow cytometry. The number of CD3⁺Vα24Jα18⁺ cells in an aliquot can be determined and a volume comprising an appropriate dose of iNKTs administered to the recipient. In some embodiments, dose calculations are adjusted based on measures of cell viability measurements, e.g., viability determined via flow cytometry with propidium iodide or 7-AAD, or via trypan blue exclusion.

A cell population of the disclosure can comprise a population of Tmems. A population of Tmems can be sourced from peripheral blood. A population of Tmems can be sourced from a peripheral blood apheresis product.

A population of cells can be enriched for Tmems, for example, by sorting based on the expression of various markers using MACS, FACS, or a combination thereof. A population of Tmems can be enriched, for example, by sorting for CD3⁺ CD45RA−CD45RO⁺ cells.

The number of Tmems present in a population can be quantified, for example, by quantifying CD3⁺ CD45RA−CD45RO⁺ cells via flow cytometry. The number of CD3⁺CD45RA−CD45RO⁺ cells in an aliquot can be determined and a volume comprising an appropriate dose of Tmems administered to the recipient. Dose calculations can be adjusted based on measures of cell viability measurements, e.g., viability determined via flow cytometry with propidium iodide or 7-AAD, or via trypan blue exclusion.

A cell population of the disclosure or a cell population of the disclosure can be administered freshly after isolation, or after cryopreservation and subsequent thawing.

Cells freshly isolated from a donor (“fresh cells”) can be administered to a recipient subject. Fresh cells can be stored in a buffer, for example, CliniMACs PBS-EDTA Buffer with 0.5% human serum albumin, or Plasma-Lyte-A, pH 7.4 supplemented with 2% human serum albumin. Fresh cells can be stored at a reduced temperature (e.g., 2-8° C.), and without being cryopreserved/frozen.

After acquiring a fresh population of cells from a donor, the fresh cells can be stored for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 44 hours, at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to administration to a subject.

After acquiring a fresh population of cells from a donor, the fresh cells can be stored for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 16 hours, at most about 18 hours, at most about 20, at most 22 hours, at most about 24 hours, at most about 30 hours, at most about 36 hours, at most about 40 hours, at most about 48 hours, at most about 60 hours, at most about 70 hours, at most about 72 hours, at most about 80 hours, at most about 90 hours, at most about 96 hours, at most about 120 hours, at most about 150 hours, at most about 200 hours, or at most about 300 hours prior to administration to a subject.

In some embodiments, after processing, one or more cell populations of the present disclosure, e.g., the first population of CD45⁺ cells, the second population of CD45⁺ cells, and/or the third population of CD45⁺ cells, may be formulated with one or more excipients and/or cryoprotectants. In some embodiments, the one or more excipients comprise buffers, such as transport or infusion buffers. In some embodiments, the cell populations are formulated at a neutral pH. In some embodiments, the HSPCs of the present disclosure are formulated with one or more excipients at a neutral pH. In some embodiments, the Tregs of the present disclosure are formulated with one or more excipients at a neutral pH. In some embodiments, the Tcons of the present disclosure are formulated with one or more excipients at a neutral pH. In some embodiments, the HSPCs and Tregs of the present disclosure are formulated together with one or more excipients at a neutral pH. In some embodiments, the HSPCs and Tcons of the present disclosure are formulated together with one or more excipients at a neutral pH. In some embodiments, the Tregs and Tcons of the present disclosure are formulated together with one or more excipients at a neutral pH. In some embodiments, the HSPCs, Tregs, and Tcons of the present disclosure are formulated together with one or more excipients at a neutral pH. In some embodiments, the one or more excipients comprise one or more transport buffers.

In some embodiments, a neutral pH ranges from approximately 6.8 to approximately 7.6. In some embodiments, a neutral pH is approximately 6.8, approximately 6.85, approximately 6.9, approximately 6.95, approximately 7, approximately 7.05, approximately 7.1, approximately 7.15, approximately 7.2, approximately 7.25, approximately 7.3, approximately 7.35, approximately 7.4, approximately 7.45, approximately 7.5, approximately 7.55, or approximately 7.6.

In some embodiments, one or more cell populations of the present disclosure, e.g., the first population of CD45⁺ cells, the second population of CD45⁺ cells, and/or the third population of CD45⁺ cells, may be formulated in one or more excipients. In some embodiments, the one or more excipients may comprise one or more buffers. The buffers may be transport buffers or infusion buffers. Transport buffers suitable for use with the cell populations of the present disclosure include buffers that have a milliequivalent (mEq) of sodium that ranges from approximately 120 mEq sodium to approximately 160 mEq sodium. In some embodiments, a transport buffer of the present disclosure comprises approximately 120 mEq sodium, approximately 125 mEq sodium, approximately 130 mEq sodium, approximately 135 mEq sodium, approximately 140 mEq sodium, approximately 145 mEq sodium, approximately 120 mEq sodium, approximately 150 mEq sodium, approximately 155 mEq sodium, or approximately 160 mEq sodium.

Transport buffers suitable for use with the cell populations of the present disclosure include buffers that have an osmotic concentration that ranges from approximately 270 mOsmol/L to approximately 320 mOsmol/L. In some embodiments, a transport buffer of the present disclosure have an osmotic concentration that is approximately 270 mOsmol/L, approximately 275 mOsmol/L, approximately 280 mOsmol/L, approximately 285 mOsmol/L, approximately 290 mOsmol/L, approximately 295 mOsmol/L, approximately 300 mOsmol/L, approximately 305 mOsmol/L, approximately 310 mOsmol/L, approximately 315 mOsmol/L, or approximately 320 mOsmol/L.

In some embodiments, transport buffers suitable for use with one or more cell populations of the present disclosure e.g., the first population of CD45⁺ cells, the second population of CD45⁺ cells, and/or the third population of CD45⁺ cells, include, without limitation, phosphate-buffered saline (PBS), human serum, PlasmaLyte, and any combination thereof. In some embodiments, one or more cell populations of the present disclosure, e.g., the first population of CD45⁺ cells, the second population of CD45⁺ cells, and/or the third population of CD45⁺ cells are formulated with a transport buffer. In some embodiments, one or more cell populations of the present disclosure, e.g., the first population of CD45⁺ cells, the second population of CD45⁺ cells, and/or the third population of CD45⁺ cells are formulated with a sufficient amount of PBS. In some embodiments, one or more cell populations of the present disclosure, e.g., the first population of CD45⁺ cells, the second population of CD45⁺ cells, and/or the third population of CD45⁺ cells are formulated with a sufficient amount of human serum. In some embodiments, one or more cell populations of the present disclosure, e.g., the first population of CD45⁺ cells, the second population of CD45⁺ cells, and/or the third population of CD45⁺ cells are formulated with a sufficient amount of PlasmaLyte (e.g., PlasmaLyte A).

In some embodiments, a transport buffer of the present disclosure may further include a human carrier protein, including without limitation, human serum albumin (HSA), intravenous immune globulin (IVIG), AB serum, or any combination thereof. In some embodiments, the human carrier protein has a concentration that ranges from approximately 0.1% weight by volume to approximately 10% weight by volume of the human carrier protein. In some embodiments, the human carrier protein has a concentration that is approximately 0.1% weight by volume, approximately 0.2% weight by volume, approximately 0.3% weight by volume, approximately 0.4% weight by volume, approximately 0.5% weight by volume, approximately 0.6% weight by volume, approximately 0.7% weight by volume, approximately 0.8% weight by volume, approximately 0.9 weight by volume, approximately 1.0% weight by volume, approximately 1.1% weight by volume, approximately 1.2% weight by volume, approximately 1.3% weight by volume, approximately 1.4% weight by volume, approximately 1.5% weight by volume, approximately 1.6% weight by volume, approximately 1.7% weight by volume, approximately 1.8% weight by volume, approximately 1.9% weight by volume, approximately 2.0% weight by volume, approximately 2.1% weight by volume, approximately 2.2% weight by volume, approximately 2.3% weight by volume, approximately 2.4% weight by volume, approximately 2.5% weight by volume, approximately 2.6% weight by volume, approximately 2.7% weight by volume, approximately 2.8% weight by volume, approximately 2.9% weight by volume, approximately 3.0% weight by volume, approximately 3.1% weight by volume, approximately 3.2% weight by volume, approximately 3.3% weight by volume, approximately 3.4% weight by volume, approximately 3.5% weight by volume, approximately 3.6% weight by volume, approximately 3.7% weight by volume, approximately 3.8% weight by volume, approximately 3.9% weight by volume, approximately 4.0% weight by volume, approximately 4.1% weight by volume, approximately 4.2% weight by volume, approximately 4.3% weight by volume, approximately 4.4% weight by volume, approximately 4.5% weight by volume, approximately 4.6% weight by volume, approximately 4.7% weight by volume, approximately 4.8% weight by volume, approximately 4.9% weight by volume, approximately 5.0% weight by volume, approximately 5.1% weight by volume, approximately 5.2% weight by volume, approximately 5.3% weight by volume, approximately 5.4% weight by volume, approximately 5.5% weight by volume, approximately 5.6% weight by volume, approximately 5.7% weight by volume, approximately 5.8% weight by volume, approximately 5.9% weight by volume, approximately 6.0% weight by volume, approximately 6.1% weight by volume, approximately 6.2% weight by volume, approximately 6.3% weight by volume, approximately 6.4% weight by volume, approximately 6.5% weight by volume, approximately 6.6% weight by volume, approximately 6.7% weight by volume, approximately 6.8% weight by volume, approximately 6.9% weight by volume, approximately 7.0% weight by volume, approximately 7.1% weight by volume, approximately 7.2% weight by volume, approximately 7.3% weight by volume, approximately 7.4% weight by volume, approximately 7.5% weight by volume, approximately 7.6% weight by volume, approximately 7.7% weight by volume, approximately 7.8% weight by volume, approximately 7.9% weight by volume, approximately 8.0% weight by volume, approximately 8.1% weight by volume, approximately 8.2% weight by volume, approximately 8.3% weight by volume, approximately 8.4% weight by volume, approximately 8.5% weight by volume, approximately 8.6% weight by volume, approximately 8.7% weight by volume, approximately 8.8% weight by volume, approximately 8.9% weight by volume, approximately 9.0% weight by volume, approximately 9.1% weight by volume, approximately 9.2% weight by volume, approximately 9.3% weight by volume, approximately 9.4% weight by volume, approximately 9.5% weight by volume, approximately 9.6% weight by volume, approximately 9.7% weight by volume, approximately 9.8% weight by volume, approximately 9.9% weight by volume, or approximately 10% weight by volume of the human carrier protein.

In some embodiments, one or more cell populations of the present disclosure, e.g., the first population of CD45⁺ cells, the second population of CD45⁺ cells, and/or the third population of CD45⁺ cells are formulated with an excipient, such as a transport buffer of the present disclosure, in an amount sufficient to yield a volume that ranges from approximately 5 mL to 1 L. In some embodiments, the first population of CD45⁺ cells is formulated with an excipient, such as a transport buffer of the present disclosure, in an amount sufficient to yield a volume that ranges from approximately 5 mL to 1 L. In some embodiments, the second population of CD45⁺ cells is formulated with an excipient, such as a transport buffer of the present disclosure, in an amount sufficient to yield a volume that ranges from approximately 5 mL to 1 L. In some embodiments, the third population of CD45⁺ cells is formulated with an excipient, such as a transport buffer of the present disclosure, in an amount sufficient to yield a volume that ranges from approximately 5 mL to 1 L. In some embodiments, the first population of CD45⁺ cells, the second population of CD45⁺ cells, and/or the third population of CD45⁺ cells is formulated (e.g., with one or more excipients, such as a transport buffer) at volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 105 mL, approximately 110 mL, approximately 115 mL, approximately 120 mL, approximately 125 mL, approximately 130 mL, approximately 135 mL, approximately 140 mL, approximately 145 mL, approximately 150 mL, approximately 155 mL, approximately 160 mL, approximately 165 mL, approximately 170 mL, approximately 175 mL, approximately 180 mL, approximately 185 mL, approximately 190 mL, approximately 195 mL, approximately 200 mL, approximately 205 mL, approximately 210 mL, approximately 215 mL, approximately 220 mL, approximately 225 mL, approximately 230 mL, approximately 235 mL, approximately 240 mL, approximately 245 mL, approximately 250 mL, approximately 255 mL, approximately 260 mL, approximately 265 mL, approximately 270 mL, approximately 275 mL, approximately 280 mL, approximately 285 mL, approximately 290 mL, approximately 295 mL, approximately 300 mL, approximately 305 mL, approximately 310 mL, approximately 315 mL, approximately 320 mL, approximately 325 mL, approximately 330 mL, approximately 335 mL, approximately 340 mL, approximately 345 mL, approximately 350 mL, approximately 355 mL, approximately 360 mL, approximately 365 mL, approximately 370 mL, approximately 375 mL, approximately 380 mL, approximately 385 mL, approximately 390 mL, approximately 395 mL, approximately 400 mL, approximately 405 mL, approximately 410 mL, approximately 415 mL, approximately 420 mL, approximately 425 mL, approximately 430 mL, approximately 435 mL, approximately 440 mL, approximately 445 mL, approximately 450 mL, approximately 455 mL, approximately 460 mL, approximately 465 mL, approximately 470 mL, approximately 475 mL, approximately 480 mL, approximately 485 mL, approximately 490 mL, approximately 495 mL, approximately 500 mL, approximately 505 mL, approximately 510 mL, approximately 515 mL, approximately 520 mL, approximately 525 mL, approximately 530 mL, approximately 535 mL, approximately 540 mL, approximately 545 mL, approximately 550 mL, approximately 555 mL, approximately 560 mL, approximately 565 mL, approximately 570 mL, approximately 575 mL, approximately 580 mL, approximately 585 mL, approximately 590 mL, approximately 595 mL, approximately 600 mL, approximately 605 mL, approximately 610 mL, approximately 615 mL, approximately 620 mL, approximately 625 mL, approximately 630 mL, approximately 635 mL, approximately 640 mL, approximately 645 mL, approximately 650 mL, approximately 655 mL, approximately 660 mL, approximately 665 mL, approximately 670 mL, approximately 675 mL, approximately 680 mL, approximately 685 mL, approximately 690 mL, approximately 695 mL, approximately 700 mL, approximately 705 mL, approximately 710 mL, approximately 715 mL, approximately 720 mL, approximately 725 mL, approximately 730 mL, approximately 735 mL, approximately 740 mL, approximately 745 mL, approximately 750 mL, approximately 755 mL, approximately 760 mL, approximately 765 mL, approximately 770 mL, approximately 775 mL, approximately 780 mL, approximately 785 mL, approximately 790 mL, approximately 795 mL, approximately 800 mL, approximately 805 mL, approximately 810 mL, approximately 815 mL, approximately 820 mL, approximately 825 mL, approximately 830 mL, approximately 835 mL, approximately 840 mL, approximately 845 mL, approximately 850 mL, approximately 855 mL, approximately 860 mL, approximately 865 mL, approximately 870 mL, approximately 875 mL, approximately 880 mL, approximately 885 mL, approximately 890 mL, approximately 895 mL, approximately 900 mL, approximately 905 mL, approximately 910 mL, approximately 915 mL, approximately 920 mL, approximately 925 mL, approximately 930 mL, approximately 935 mL, approximately 940 mL, approximately 945 mL, approximately 950 mL, approximately 955 mL, approximately 960 mL, approximately 965 mL, approximately 970 mL, approximately 975 mL, approximately 980 mL, approximately 985 mL, approximately 990 mL, approximately 995 mL, or approximately 1 L. In some embodiments, a formulation of the present disclosure further comprises a human carrier protein of the present disclosure. In some embodiments, a formulation of the present disclosure further comprises one or more cryoprotectants.

Cells populations of the present disclosure, e.g., the first population of CD45⁺ cells, the second population of CD45⁺ cells, and/or the third population of CD45⁺ cells, can be cryopreserved. In some embodiments, cryopreservation can be beneficial to the methods disclosed herein. For example, cryopreservation of the third population of CD45⁺ cells that comprises, at least, Tcons prior to subsequent thawing and administering to a subject may reduce GVHD.

An additional aspect provides a method of transplanting a conventional T cell (Tcon) population into a human subject without eliciting a stage 2 or higher graft versus host disease (GVHD) response up to approximately 100 days after transplanting. The method comprising: (i). administering a solution comprising a population of conventional T cells (Tcons); and (ii). administering a solution comprising a population of regulatory T cells (Tregs). In this method, the population of Tcons is cryopreserved for at least about 4 hours; and the solution comprising the population of Tcons and the solution comprising the population of Tregs comprise less than about 5 EU of endotoxins per ml of the solution.

Cryopreservation can comprise addition of a preservative agent (e.g., DMSO) or one or more cryoprotectants, and gradual cooling of cells in a controlled-rate freezer to prevent osmotic cellular injury resulting from ice crystal formation. Cryopreservation can comprise commercial cryopreservation reagents and materials (e.g., cryoprotectants), for example, Cryobags sorbitol, dimethyl sulfoxide (DMSO), propylene glycol, glycerol, polyvinylpyrrolidone (PVP), and polyethylene glycol (PEG), serum, HSA, hetastarch, CRYOSTOR CS2, CRYOSTOR CS5, and CRYOSTOR CS10, or any combination thereof.

In certain embodiments, a cryoprotectant of the present disclosure comprises DMSO. For example, in some embodiments the third population of CD45⁺ cells may be formulated for cryopreservation with a cryoprotectant that comprises DMSO in an amount that ranges from approximately 1% volume by volume to approximately 15% volume by volume. In some embodiments, the cryoprotectant comprises DMSO in an amount that is approximately 1% volume by volume, approximately 1.2% volume by volume, approximately 1.4% volume by volume, approximately 1.6% volume by volume, approximately 1.8% volume by volume, approximately 2% volume by volume, approximately 2.2% volume by volume, approximately 2.4% volume by volume, approximately 2.6% volume by volume, approximately 2.8% volume by volume, approximately 3% volume by volume, approximately 3.2% volume by volume, approximately 3.4% volume by volume, approximately 3.6% volume by volume, approximately 3.8% volume by volume, approximately 4% volume by volume, approximately 4.2% volume by volume, approximately 4.4% volume by volume, approximately 4.6% volume by volume, approximately 4.8% volume by volume, approximately 5% volume by volume, approximately 5.2% volume by volume, approximately 5.4% volume by volume, approximately 5.6% volume by volume, approximately 5.8% volume by volume, approximately 6% volume by volume, approximately 6.2% volume by volume, approximately 6.4% volume by volume, approximately 6.6% volume by volume, approximately 6.8% volume by volume, approximately 7% volume by volume, approximately 7.2% volume by volume, approximately 7.4% volume by volume, approximately 7.6% volume by volume, approximately 7.8% volume by volume, approximately 8% volume by volume, approximately 8.2% volume by volume, approximately 8.4% volume by volume, approximately 8.6% volume by volume, approximately 8.8% volume by volume, approximately 9% volume by volume, approximately 9.2% volume by volume, approximately 9.4% volume by volume, approximately 9.6% volume by volume, approximately 9.8% volume by volume, approximately 10% volume by volume, approximately 10.2% volume by volume, approximately 10.4% volume by volume, approximately 10.6% volume by volume, approximately 10.8% volume by volume, approximately 11% volume by volume, approximately 11.2% volume by volume, approximately 11.4% volume by volume, approximately 11.6% volume by volume, approximately 11.8% volume by volume, approximately 12% volume by volume, approximately 12.2% volume by volume, approximately 12.4% volume by volume, approximately 12.6% volume by volume, approximately 12.8% volume by volume, approximately 13% volume by volume, approximately 13.2% volume by volume, approximately 13.4% volume by volume, approximately 13.6% volume by volume, approximately 13.8% volume by volume, approximately 14% volume by volume, approximately 14.2% volume by volume, approximately 14.4% volume by volume, approximately 14.6% volume by volume, approximately 14.8% volume by volume, or approximately 15% volume by volume.

In some embodiments, the third population of CD45⁺ cells may be formulated for cryopreservation with a cryoprotectant that comprises DMSO, and that further comprises hetastarch. In some embodiments, the cryoprotectant comprises hetastarch in an amount that ranges from approximately 0.1% weight by volume to approximately 5% weight by volume. In some embodiments, the cryoprotectant comprises hetastarch in an amount that is approximately 0.1% weight by volume, approximately 0.2% weight by volume, approximately 0.3% weight by volume, approximately 0.4% weight by volume, approximately 0.5% weight by volume, approximately 0.6% weight by volume, approximately 0.7% weight by volume, approximately 0.8% weight by volume, approximately 0.9 weight by volume, approximately 1.0% weight by volume, approximately 1.1% weight by volume, approximately 1.2% weight by volume, approximately 1.3% weight by volume, approximately 1.4% weight by volume, approximately 1.5% weight by volume, approximately 1.6% weight by volume, approximately 1.7% weight by volume, approximately 1.8% weight by volume, approximately 1.9% weight by volume, approximately 2.0% weight by volume, approximately 2.1% weight by volume, approximately 2.2% weight by volume, approximately 2.3% weight by volume, approximately 2.4% weight by volume, approximately 2.5% weight by volume, approximately 2.6% weight by volume, approximately 2.7% weight by volume, approximately 2.8% weight by volume, approximately 2.9% weight by volume, approximately 3.0% weight by volume, approximately 3.1% weight by volume, approximately 3.2% weight by volume, approximately 3.3% weight by volume, approximately 3.4% weight by volume, approximately 3.5% weight by volume, approximately 3.6% weight by volume, approximately 3.7% weight by volume, approximately 3.8% weight by volume, approximately 3.9% weight by volume, approximately 4.0% weight by volume, approximately 4.1% weight by volume, approximately 4.2% weight by volume, approximately 4.3% weight by volume, approximately 4.4% weight by volume, approximately 4.5% weight by volume, approximately 4.6% weight by volume, approximately 4.7% weight by volume, approximately 4.8% weight by volume, approximately 4.9% weight by volume, or approximately 5.0% weight by volume.

Cryopreserved cells can be stored for periods of time ranging from hours to years at low temperatures. Cryopreserved cells can be stored at ultralow temperatures, for example, −50° C., −60° C., −70° C., −80° C., −90° C., −100° C., −110° C., −120° C., −130° C., −140° C., −150° C., −160° C., −170° C., −180° C., −190° C., −196° C., or less. Cryopreserved cells can be stored in storage devices comprising liquid nitrogen.

Cells can be cryopreserved before or after certain steps in the methods of the disclosure, for example, before or after sorting steps, before or after characterization steps, such as determining cell viability or the concentration of cells of a particular type.

In some embodiments, whole blood can be cryopreserved. Whole blood can be cryopreserved without sorting or characterization. Whole blood can be cryopreserved after sorting but without characterization. Whole blood can be cryopreserved after characterization but without sorting. Whole blood can be cryopreserved after characterization and sorting. Whole blood can be cryopreserved after quantifying a cell type of the disclosure Whole blood can be cryopreserved after quantifying conventional T cells (Tcons, e.g., CD3⁺ cells). Whole blood can be cryopreserved after quantifying viability of all cells or a population of cells of the disclosure (e.g., conventional T cells).

A peripheral blood apheresis product of the disclosure can be cryopreserved. A peripheral blood apheresis product can be cryopreserved without sorting or characterization. A peripheral blood apheresis product can be cryopreserved after sorting but without characterization. A peripheral blood apheresis product can be cryopreserved after characterization but without sorting. A peripheral blood apheresis product can be cryopreserved after characterization and sorting. A peripheral blood apheresis product can be cryopreserved after quantifying a cell type of the disclosure. A peripheral blood apheresis product can be cryopreserved after quantifying conventional T cells (Tcons, e.g., CD3⁺ cells). A peripheral blood apheresis product can be cryopreserved after quantifying viability of all cells or a population of cells of the disclosure (e.g., conventional T cells).

A population of cells sorted or selected from another population of cells can be cryopreserved, for example, a population of CD45⁺ cells, HSPCs, Tregs, Tcons, iNKTs, or Tmems can be cryopreserved.

A cell population of the disclosure can be cryopreserved for any amount of time. Cells of the disclosure may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, a cell population of the disclosure is cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, a cell population of the disclosure is cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, a cell population of the disclosure is cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

In some embodiments, once formulated, one or more cell populations of the present disclosure, e.g., the first population of CD45⁺ cells, the second population of CD45⁺ cells, and/or the third population of CD45⁺ cells, may be placed into one or more containers. In certain aspects, the present disclosure related to a system comprising one or more containers containing one or more cell populations of the present disclosure, e.g., the first population of CD45⁺ cells, the second population of CD45⁺ cells, and/or the third population of CD45⁺ cells. In some embodiments, the one or more containers are filled to an appropriate volume (e.g., 5 mL to 1 L) with one or more excipients of the present disclosure, such as one or more transport buffers, one or more human carrier proteins, and/or one or more cryoprotectants, at a suitable pH, e.g., a neutral pH. In some embodiments, a single container contains the first population of CD45⁺ cells, the second population of CD45⁺ cells, and the third population of CD45⁺ cells. In some embodiments, a first container contains the first population of CD45⁺ cells and the second population of CD45⁺ cells, and a second container contains the third population of CD45⁺ cells. In some embodiments, a first container contains the first population of CD45⁺ cells and the third population of CD45⁺ cells, and a second container contains the second population of CD45⁺ cells. In some embodiments, a first container contains the second population of CD45⁺ cells and the third population of CD45⁺ cells, and a second container contains the first population of CD45⁺ cells. In some embodiments, a first container contains the first population of CD45⁺ cells, a second container contains the second population of CD45⁺ cells, and a third container contains the third population of CD45⁺ cells. In some embodiments, a container of the present disclosure includes, without limitation, a vial, a syringe, a bag (e.g., transfer bag), a cryoprotectant container, and any combinations thereof.

In some embodiments, the container is a bag, such as a transfer bag, that can be used, for example, for infusion. In some embodiments, a transfer bag of the present disclosure is a single dose transfer bag. In some embodiments, a transfer bag of the present disclosure is a polyvinyl chloride (PVC) transfer bag or an ethylene vinyl acetate (EVA) transfer bag.

Certain aspects of the present disclosure relate to systems, e.g., a cellular therapy or treatment systems that comprises one or more of the containers of the present disclosure. Other aspects of the present disclosure relate to articles of manufacture comprising one or more of the containers of the present disclosure. Other aspects of the present disclosure relate to kits comprising one or more of the containers of the present disclosure.

### Donors

In some embodiments, one or more cell populations of the present disclosure, e.g., a first population of CD45⁺ cells of the present disclosure, a second population of CD45⁺ cells of the present disclosure, and/or a third population of CD45⁺ cells of the present disclosure, are derived from a single human blood donor. In embodiments, the respective cell populations are provided as separate cell populations and are derived from a single human blood donor.

A cell population can comprise cells that are from one or more donors that have each been HLA typed, for example, to determine a degree of HLA matching to a subject that will receive the cell population. In some embodiments, the donor is unrelated to the recipient subject. In some embodiments, the donor is related to a recipient subject, for example, the donor is a parent, child, sibling, grandparent, grandchild, aunt, uncle, or cousin. In some embodiments, the donor is a first-degree blood relative of the recipient subject. In some embodiments, the donor is a second-degree blood relative of the recipient subject. In some embodiments, the related or unrelated donor is HLA matched to a recipient subject. In some embodiments, the related or unrelated donor is HLA mismatched to a recipient subject. In some embodiments, the related or unrelated donor is haploidentical to a recipient subject. In some embodiments, the related or unrelated donor is at least 16 years old. In some embodiments, the related or unrelated donor is at least 18 years old.

Human leukocyte antigens (HLA), also broadly referred to as Major histocompatibility complex (MHC) antigens, can be protein molecules expressed on the surface of a cell that can confer an antigenic identity to that cell. HLA/MHC antigens are target molecules that can be recognized by T cells and natural killer (NK) cells as being derived from the same source of hematopoietic stem cells as the immune effector cells (“self”), or as being derived from another source of hematopoietic cells (“non-self”). HLA class I antigens (A, B, and C in humans) can be expressed by the vast majority of cells, while HLA class II antigens (DR, DP, and DQ in humans) can be expressed primarily on professional antigen presenting cells. Both HLA classes can be implicated in GVHD.

HLA antigens are encoded by highly polymorphic genes; a range of alleles exist for each HLA class I and II gene. Allelic gene products can differ in one or more amino acids in the α and/or β domain(s). Panels of specific antibodies or nucleic acid reagents can be used to determine HLA haplotypes of individuals, for example, using leukocytes that express class I and class II molecules. HLA alleles can be described at various levels of detail. Most designations begin with HLA- and the locus name, then * and some (even) number of digits specifying the allele. The first two digits can specify a group of alleles. The third through fourth digits, when present, can specify a synonymous allele. Digits five through six, when present, can denote any synonymous mutations within the coding frame of the gene. The seventh and eighth digits, when present, can distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

The set of HLA alleles inherited from one parent forms a haplotype. HLA haploidentical can refer to a donor-recipient pair where one chromosome is matched at least at HLA-A; HLA-B, and HLA-DR between the donor and recipient. The haploidentical pair may or may not be matched at other alleles, e.g., other HLA genes on the other chromosome, or additional histocompatibility loci on either chromosome. Such donors can frequently occur in families, e.g., a parent can be haploidentical to a child; and siblings may be haploidentical.

A cell population can be from a related or unrelated donor that has been HLA-typed at any number of HLA alleles. A donor and a subject can be HLA matched, e.g., matched at all typed HLA alleles. A donor and a subject can be HLA mismatched, e.g., at least one HLA antigen can be mismatched between the donor and recipient.

In some embodiments, a related or unrelated donor and a subject can be HLA-typed at six alleles, for example, HLA-A, HLA-B, and HLA-DR alleles. The donor and subject can be matched at, for example 3/6 4/6, 5/6, or 6/6 of the alleles. In some embodiments, the donor and subject are matched at least at 5/6 alleles. In some embodiments, the donor and subject are matched at 6/6 alleles.

In some embodiments, a related or unrelated donor and a subject can be HLA-typed at eight alleles, for example, HLA-A, HLA-B, HLA-C, and HLA-DR alleles (e.g., HLA-DRB1 alleles). The donor and subject can be matched at, for example 4/8, 5/8, 6/8, 7/8, or 8/8 of the alleles. In some embodiments, the donor and subject are matched at least at 6/8 alleles. In some embodiments, the donor and subject are matched at least at 7/8 alleles. In some embodiments, the donor and subject are matched at 8/8 alleles.

In some embodiments, a related or unrelated donor and a subject can be HLA-typed at ten alleles, for example, HLA-A, HLA-B, HLA-C, and HLA-DR alleles (e.g., HLA-DRB1 alleles). The donor and subject can be matched at, for example 5/10, 6/10, 7/10, 8/10, 9/10, or 10/10 of the alleles. In some embodiments, the donor and subject are matched at least at 7/10 alleles. In some embodiments, the donor and subject are matched at least at 8/10 alleles. In some embodiments, the donor and subject are matched at least at 9/10 alleles. In some embodiments, the donor and subject are matched at 10/10 alleles.

In some embodiments, a related or unrelated donor and a subject can be HLA-typed at twelve alleles, for example, HLA-A, HLA-B, HLA-C, HLA-DR alleles (e.g., HLA-DRB1 alleles), and HLA-DP alleles (e.g., HLA-DPB1 alleles). The donor and subject can be matched at, for example 6/12, 7/12, 8/12, 9/12, 10/12, 11/12, or 12/12 of the alleles. In some embodiments, the donor and subject are matched at least at 9/12 alleles. In some embodiments, the donor and subject are matched at least at 10/12 alleles. In some embodiments, the donor and subject are matched at least at 11/12 alleles. In some embodiments, the donor and subject are matched at 12/12 alleles.

A cell population can be generated from a matched unrelated donor that is an 8/8 match for HLA-A, -B, -C, and -DRB1, all typed using DNA-based high-resolution methods. A cell population can be generated from a matched unrelated donor that is an 7/8 match for HLA-A, -B, -C, and -DRB1, all typed using DNA-based high-resolution methods. A cell population can be generated from a matched unrelated donor that is an 6/8 match for HLA-A, -B, -C, and -DRB1, all typed using DNA-based high-resolution methods. A cell population can be generated from a matched unrelated donor that is an 10/10 match for HLA-A, -B, -C, -DQB1 and -DRB1, all typed using DNA-based high-resolution methods. A cell population can be generated from a matched unrelated donor that is an 9/10 match for HLA-A, -B, -C, -DQB1 and -DRB1, all typed using DNA-based high-resolution methods. A cell population can be generated from a matched unrelated donor that is an 8/10 match for HLA-A, -B, -C, -DQB1 and -DRB1, all typed using DNA-based high-resolution methods. A cell population can be generated from a matched unrelated donor that is an 7/10 match for HLA-A, -B, -C, -DQB1 and -DRB1, all typed using DNA-based high-resolution methods. A cell population can be generated from a matched unrelated donor that is an 12/12 match for HLA-A, -B, -C, -DQB1, -DRB1, and -DPB1, all typed using DNA-based high-resolution methods. A cell population can be generated from a matched unrelated donor that is an 11/12 match for HLA-A, -B, -C, -DQB1, -DRB1, and -DPB1, all typed using DNA-based high-resolution methods. A cell population can be generated from a matched unrelated donor that is an 10/12 match for HLA-A, -B, -C, -DQB1, -DRB1, and -DPB1, all typed using DNA-based high-resolution methods. A cell population can be generated from a matched unrelated donor that is an 9/12 match for HLA-A, -B, -C, -DQB1, -DRB1, and -DPB1, all typed using DNA-based high-resolution methods.

A cell population can be generated from a matched related donor that is an 8/8 match for HLA-A, -B, -C, and -DRB1, all typed using DNA-based high-resolution methods. A cell population can be generated from a matched related donor that is an 7/8 match for HLA-A, -B, -C, and -DRB1, all typed using DNA-based high-resolution methods. A cell population can be generated from a matched related donor that is an 6/8 match for HLA-A, -B, -C, and -DRB1, all typed using DNA-based high-resolution methods. A cell population can be generated from a matched related donor that is an 10/10 match for HLA-A, -B, -C, -DQB1, and -DRB1, all typed using DNA-based high-resolution methods. A cell population can be generated from a matched related donor that is an 9/10 match for HLA-A, -B, -C, -DQB1, and -DRB1, all typed using DNA-based high-resolution methods. A cell population can be generated from a matched related donor that is an 8/10 match for HLA-A, -B, -C, -DQB1, and -DRB1, all typed using DNA-based high-resolution methods. A cell population can be generated from a matched related donor that is an 7/10 match for HLA-A, -B, -C, -DQB1, and -DRB1, all typed using DNA-based high-resolution methods. A cell population can be generated from a matched related donor that is an 12/12 match for HLA-A, -B, -C, -DQB1, -DRB1, and -DPB1, all typed using DNA-based high-resolution methods. A cell population can be generated from a matched related donor that is an 11/12 match for HLA-A, -B, -C, -DQB1, -DRB1, and -DPB1, all typed using DNA-based high-resolution methods. A cell population can be generated from a matched related donor that is an 10/12 match for HLA-A, -B, -C, -DQB1, -DRB1, and -DPB1, all typed using DNA-based high-resolution methods. A cell population can be generated from a matched related donor that is an 9/12 match for HLA-A, -B, -C, -DQB1, -DRB1, and -DPB1, all typed using DNA-based high-resolution methods.

A cell population can be generated from a matched parent donor that is an 8/8 match for HLA-A, -B, -C, and -DRB1, all typed using DNA-based high-resolution methods. A cell population can be generated from a matched parent donor that is an 7/8 match for HLA-A, -B, -C, and -DRB1, all typed using DNA-based high-resolution methods. A cell population can be generated from a matched parent donor that is an 6/8 match for HLA-A, -B, -C, and -DRB1, all typed using DNA-based high-resolution methods. A cell population can be generated from a matched parent donor that is an 10/10 match for HLA-A, -B, -C, -DQB1, and -DRB1, all typed using DNA-based high-resolution methods. A cell population can be generated from a matched parent donor that is an 9/10 match for HLA-A, -B, -C, -DQB1, and -DRB1, all typed using DNA-based high-resolution methods. A cell population can be generated from a matched parent donor that is an 8/10 match for HLA-A, -B, -C, -DQB1, and -DRB1, all typed using DNA-based high-resolution methods. A cell population can be generated from a matched parent donor that is an 7/10 match for HLA-A, -B, -C, -DQB1, and -DRB1, all typed using DNA-based high-resolution methods. A cell population can be generated from a matched parent donor that is an 12/12 match for HLA-A, -B, -C, -DQB1, -DRB1, and -DPB1, all typed using DNA-based high-resolution methods. A cell population can be generated from a matched parent donor that is an 11/12 match for HLA-A, -B, -C, -DQB1, -DRB1, and -DPB1, all typed using DNA-based high-resolution methods. A cell population can be generated from a matched parent donor that is an 10/12 match for HLA-A, -B, -C, -DQB1, -DRB1, and -DPB1, all typed using DNA-based high-resolution methods. A cell population can be generated from a matched parent donor that is an 9/12 match for HLA-A, -B, -C, -DQB1, -DRB1, and -DPB1, all typed using DNA-based high-resolution methods.

A cell population can be generated from a matched child donor that is an 8/8 match for HLA-A, -B, -C, and -DRB1, all typed using DNA-based high-resolution methods. A cell population can be generated from a matched child donor that is an 7/8 match for HLA-A, -B, -C, and -DRB1, all typed using DNA-based high-resolution methods. A cell population can be generated from a matched child donor that is an 6/8 match for HLA-A, -B, -C, and -DRB1, all typed using DNA-based high-resolution methods. A cell population can be generated from a matched child donor that is an 10/10 match for HLA-A, -B, -C, -DQB1, and -DRB1, all typed using DNA-based high-resolution methods. A cell population can be generated from a matched child donor that is an 9/10 match for HLA-A, -B, -C, -DQB1, and -DRB1, all typed using DNA-based high-resolution methods. A cell population can be generated from a matched child donor that is an 8/10 match for HLA-A, -B, -C, -DQB1, and -DRB1, all typed using DNA-based high-resolution methods. A cell population can be generated from a matched child donor that is an 7/10 match for HLA-A, -B, -C, -DQB1, and -DRB1, all typed using DNA-based high-resolution methods. A cell population can be generated from a matched child donor that is an 12/12 match for HLA-A, -B, -C, -DQB1, -DRB1, and -DPB1, all typed using DNA-based high-resolution methods. A cell population can be generated from a matched child donor that is an 11/12 match for HLA-A, -B, -C, -DQB1, -DRB1, and -DPB1, all typed using DNA-based high-resolution methods. A cell population can be generated from a matched child donor that is an 10/12 match for HLA-A, -B, -C, -DQB1, -DRB1, and -DPB1, all typed using DNA-based high-resolution methods. A cell population can be generated from a matched child donor that is an 9/12 match for HLA-A, -B, -C, -DQB1, -DRB1, and -DPB1, all typed using DNA-based high-resolution methods.

A cell population can be generated from a matched sibling donor that is an 8/8 match for HLA-A, -B, -C, and -DRB1, all typed using DNA-based high-resolution methods. A cell population can be generated from a matched sibling donor that is an 7/8 match for HLA-A, -B, -C, and -DRB1, all typed using DNA-based high-resolution methods. A cell population can be generated from a matched sibling donor that is an 6/8 match for HLA-A, -B, -C, and -DRB1, all typed using DNA-based high-resolution methods. A cell population can be generated from a matched sibling donor that is an 10/10 match for HLA-A, -B, -C, -DQB1 and -DRB1, all typed using DNA-based high-resolution methods. A cell population can be generated from a matched sibling donor that is an 9/10 match for HLA-A, -B, -C, -DQB1 and -DRB1, all typed using DNA-based high-resolution methods. A cell population can be generated from a matched sibling donor that is an 8/10 match for HLA-A, -B, -C, -DQB1 and -DRB1, all typed using DNA-based high-resolution methods. A cell population can be generated from a matched sibling donor that is an 7/10 match for HLA-A, -B, -C, -DQB1 and -DRB1, all typed using DNA-based high-resolution methods. A cell population can be generated from a matched child donor that is an 12/12 match for HLA-A, -B, -C, -DQB1, -DRB1, and -DPB1, all typed using DNA-based high-resolution methods. A cell population can be generated from a matched child donor that is an 11/12 match for HLA-A, -B, -C, -DQB1, -DRB1, and -DPB1, all typed using DNA-based high-resolution methods. A cell population can be generated from a matched child donor that is an 10/12 match for HLA-A, -B, -C, -DQB1, -DRB1, and -DPB1, all typed using DNA-based high-resolution methods. A cell population can be generated from a matched child donor that is an 9/12 match for HLA-A, -B, -C, -DQB1, -DRB1, and -DPB1, all typed using DNA-based high-resolution methods.

A cell population can be generated from a matched grandparent donor that is an 8/8 match for HLA-A, -B, -C, and -DRB1, all typed using DNA-based high-resolution methods. A cell population can be generated from a matched grandparent donor that is an 7/8 match for HLA-A, -B, -C, and -DRB1, all typed using DNA-based high-resolution methods. A cell population can be generated from a matched grandparent donor that is an 6/8 match for HLA-A, -B, -C, and -DRB1, all typed using DNA-based high-resolution methods. A cell population can be generated from a matched grandparent donor that is an 10/10 match for HLA-A, -B, -C, -DQB1, and -DRB1, all typed using DNA-based high-resolution methods. A cell population can be generated from a matched grandparent donor that is an 9/10 match for HLA-A, -B, -C, -DQB1, and -DRB1, all typed using DNA-based high-resolution methods. A cell population can be generated from a matched grandparent donor that is an 8/10 match for HLA-A, -B, -C, -DQB1, and -DRB1, all typed using DNA-based high-resolution methods. A cell population can be generated from a matched grandparent donor that is an 7/10 match for HLA-A, -B, -C, -DQB1, and -DRB1, all typed using DNA-based high-resolution methods. A cell population can be generated from a matched grandparent donor that is an 12/12 match for HLA-A, -B, -C, -DQB1, -DRB1, and -DPB1, all typed using DNA-based high-resolution methods. A cell population can be generated from a matched grandparent donor that is an 11/12 match for HLA-A, -B, -C, -DQB1, -DRB1, and -DPB1, all typed using DNA-based high-resolution methods. A cell population can be generated from a matched grandparent donor that is an 10/12 match for HLA-A, -B, -C, -DQB1, -DRB1, and -DPB1, all typed using DNA-based high-resolution methods. A cell population can be generated from a matched grandparent donor that is an 9/12 match for HLA-A, -B, -C, -DQB1, -DRB1, and -DPB1, all typed using DNA-based high-resolution methods.

A cell population can be generated from a matched grandchild donor that is an 8/8 match for HLA-A, -B, -C, and -DRB1, all typed using DNA-based high-resolution methods. A cell population can be generated from a matched grandchild donor that is an 7/8 match for HLA-A, -B, -C, and -DRB1, all typed using DNA-based high-resolution methods. A cell population can be generated from a matched grandchild donor that is an 6/8 match for HLA-A, -B, -C, and -DRB1, all typed using DNA-based high-resolution methods. A cell population can be generated from a matched grandchild donor that is an 10/10 match for HLA-A, -B, -C, -DQB1, and -DRB1, all typed using DNA-based high-resolution methods. A cell population can be generated from a matched grandchild donor that is an 9/10 match for HLA-A, -B, -C, -DQB1, and -DRB1, all typed using DNA-based high-resolution methods. A cell population can be generated from a matched grandchild donor that is an 8/10 match for HLA-A, -B, -C, -DQB1, and -DRB1, all typed using DNA-based high-resolution methods. A cell population can be generated from a matched grandchild donor that is an 7/10 match for HLA-A, -B, -C, -DQB1, and -DRB1, all typed using DNA-based high-resolution methods. A cell population can be generated from a matched grandchild donor that is an 12/12 match for HLA-A, -B, -C, -DQB1, -DRB1, and -DPB1, all typed using DNA-based high-resolution methods. A cell population can be generated from a matched grandchild donor that is an 11/12 match for HLA-A, -B, -C, -DQB1, -DRB1, and -DPB1, all typed using DNA-based high-resolution methods. A cell population can be generated from a matched grandchild donor that is an 10/12 match for HLA-A, -B, -C, -DQB1, -DRB1, and -DPB1, all typed using DNA-based high-resolution methods. A cell population can be generated from a matched grandchild donor that is an 9/12 match for HLA-A, -B, -C, -DQB1, -DRB1, and -DPB1, all typed using DNA-based high-resolution methods.

A cell population can be generated from a matched aunt donor that is an 8/8 match for HLA-A, -B, -C, and -DRB1, all typed using DNA-based high-resolution methods. A cell population can be generated from a matched aunt donor that is an 7/8 match for HLA-A, -B, -C, and -DRB1, all typed using DNA-based high-resolution methods. A cell population can be generated from a matched aunt donor that is an 6/8 match for HLA-A, -B, -C, and -DRB1, all typed using DNA-based high-resolution methods. A cell population can be generated from a matched aunt donor that is an 10/10 match for HLA-A, -B, -C, -DQB1, and -DRB1, all typed using DNA-based high-resolution methods. A cell population can be generated from a matched aunt donor that is an 9/10 match for HLA-A, -B, -C, -DQB1, and -DRB1, all typed using DNA-based high-resolution methods. A cell population can be generated from a matched aunt donor that is an 8/10 match for HLA-A, -B, -C, -DQB1, and -DRB1, all typed using DNA-based high-resolution methods. A cell population can be generated from a matched aunt donor that is an 7/10 match for HLA-A, -B, -C, -DQB1, and -DRB1, all typed using DNA-based high-resolution methods. A cell population can be generated from a matched aunt donor that is an 12/12 match for HLA-A, -B, -C, -DQB1, -DRB1, and -DPB1, all typed using DNA-based high-resolution methods. A cell population can be generated from a matched aunt donor that is an 11/12 match for HLA-A, -B, -C, -DQB1, -DRB1, and -DPB1, all typed using DNA-based high-resolution methods. A cell population can be generated from a matched aunt donor that is an 10/12 match for HLA-A, -B, -C, -DQB1, -DRB1, and -DPB1, all typed using DNA-based high-resolution methods. A cell population can be generated from a matched aunt donor that is an 9/12 match for HLA-A, -B, -C, -DQB1, -DRB1, and -DPB1, all typed using DNA-based high-resolution methods.

A cell population can be generated from a matched uncle donor that is an 8/8 match for HLA-A, -B, -C, and -DRB1, all typed using DNA-based high-resolution methods. A cell population can be generated from a matched uncle donor that is an 7/8 match for HLA-A, -B, -C, and -DRB1, all typed using DNA-based high-resolution methods. A cell population can be generated from a matched uncle donor that is an 6/8 match for HLA-A, -B, -C, and -DRB1, all typed using DNA-based high-resolution methods. A cell population can be generated from a matched uncle donor that is an 10/10 match for HLA-A, -B, -C, -DQB1, and -DRB1, all typed using DNA-based high-resolution methods. A cell population can be generated from a matched uncle donor that is an 9/10 match for HLA-A, -B, -C, -DQB1, and -DRB1, all typed using DNA-based high-resolution methods. A cell population can be generated from a matched uncle donor that is an 8/10 match for HLA-A, -B, -C, -DQB1, and -DRB1, all typed using DNA-based high-resolution methods. A cell population can be generated from a matched uncle donor that is an 7/10 match for HLA-A, -B, -C, -DQB1, and -DRB1, all typed using DNA-based high-resolution methods. A cell population can be generated from a matched uncle donor that is an 12/12 match for HLA-A, -B, -C, -DQB1, -DRB1, and -DPB1, all typed using DNA-based high-resolution methods. A cell population can be generated from a matched uncle donor that is an 11/12 match for HLA-A, -B, -C, -DQB1, -DRB1, and -DPB1, all typed using DNA-based high-resolution methods. A cell population can be generated from a matched uncle donor that is an 10/12 match for HLA-A, -B, -C, -DQB1, -DRB1, and -DPB1, all typed using DNA-based high-resolution methods. A cell population can be generated from a matched uncle donor that is an 9/12 match for HLA-A, -B, -C, -DQB1, -DRB1, and -DPB1, all typed using DNA-based high-resolution methods.

A cell population can be generated from a matched cousin donor that is an 8/8 match for HLA-A, -B, -C, and -DRB1, all typed using DNA-based high-resolution methods. A cell population can be generated from a matched cousin donor that is an 7/8 match for HLA-A, -B, -C, and -DRB1, all typed using DNA-based high-resolution methods. A cell population can be generated from a matched cousin donor that is an 6/8 match for HLA-A, -B, -C, and -DRB1, all typed using DNA-based high-resolution methods. A cell population can be generated from a matched cousin donor that is an 10/10 match for HLA-A, -B, -C, -DQB1, and -DRB1, all typed using DNA-based high-resolution methods. A cell population can be generated from a matched cousin donor that is an 9/10 match for HLA-A, -B, -C, -DQB1, and -DRB1, all typed using DNA-based high-resolution methods. A cell population can be generated from a matched cousin donor that is an 8/10 match for HLA-A, -B, -C, -DQB1, and -DRB1, all typed using DNA-based high-resolution methods. A cell population can be generated from a matched cousin donor that is an 7/10 match for HLA-A, -B, -C, -DQB1, and -DRB1, all typed using DNA-based high-resolution methods. A cell population can be generated from a matched cousin donor that is an 12/12 match for HLA-A, -B, -C, -DQB1, -DRB1, and -DPB1, all typed using DNA-based high-resolution methods. A cell population can be generated from a matched cousin donor that is an 11/12 match for HLA-A, -B, -C, -DQB1, -DRB1, and -DPB1, all typed using DNA-based high-resolution methods. A cell population can be generated from a matched cousin donor that is an 10/12 match for HLA-A, -B, -C, -DQB1, -DRB1, and -DPB1, all typed using DNA-based high-resolution methods. A cell population can be generated from a matched cousin donor that is an 9/12 match for HLA-A, -B, -C, -DQB1, -DRB1, and -DPB1, all typed using DNA-based high-resolution methods.

In some embodiments, the HLA mismatch occurs as the result of the allogeneic related or unrelated donor being homozygous for the HLA allele while the recipient subject is heterogeneous for the HLA allele. In some embodiments, the HLA mismatch occurs as the result of the allogeneic related or unrelated donor being heterogeneous for the HLA allele while the recipient subject is homozygous for the HLA allele. In some embodiments, the HLA mismatch occurs as the result of both the allogeneic related or unrelated donor and the recipient subject being heterozygous for the HLA allele.

In various embodiments, the first population of CD45⁺ cells, the population of cells enriched for Tregs (e.g., the second population of CD45⁺ cells), and/or the third population of CD45⁺ cells is allogeneic relative to the human subject.

In some embodiments, the first population of CD45⁺ cells, the population of cells enriched for Tregs (e.g., the second population of CD45⁺ cells), and/or the third population of CD45⁺ cells is obtained from a donor that is HLA-matched relative to the human subject.

In embodiments, the first population of CD45⁺ cells, the population of cells enriched for Tregs (e.g., the second population of CD45⁺ cells), and/or the third population of CD45⁺ cells is obtained from a donor that is HLA-mismatched relative to the human subject.

In various embodiments, the first population of CD45⁺ cells, the population of cells enriched for Tregs (e.g., the second population of CD45⁺ cells), and/or the third population of CD45⁺ cells is obtained from a donor that is haploidentical relative to the human subject.

A cell population can be derived from an allogeneic donor. A cell population can be generated from a donor that is a first-degree blood relative of the subject. A cell population can be generated from a donor that is a second-degree blood relative of the subject. A cell population can be generated from a donor that is not related to the subject. A cell population can be generated from a donor that is HLA matched to a recipient subject. A cell population can be generated from a donor that is HLA mismatched to a recipient subject. A cell population can be generated from a donor that is haploidentical to a recipient subject. A cell population can be generated from a donor that is related to a recipient subject, for example, a parent, child, sibling, grandparent, grandchild, aunt, uncle, or cousin. A cell population can be generated from a donor that is at least 16 years old. A cell population can be generated from a donor that is at least 18 years old.

A cell population can be generated from a donor that meets eligibility criteria for donors of viable, leukocyte-rich cells or tissues as defined by 21 CFR § 1271 2018 and relevant FDA Guidance for Industry. For example, a cell population can be generated from a donor that meets eligibility criteria outlined in any one or more of the following: Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; and Donor Screening Recommendations to Reduce the Risk of Transmission of Zika Virus by Human Cells, Tissues, and Cellular and Tissue-Based Products, 2018). A cell population can be generated from a donor that meets any criteria for donation as specified by standard NMDP guidelines (NMDP donors).

A cell population can be generated from a donor that does not exhibit evidence of active infection. A cell population can be generated from a donor that is not seropositive for HIV-1 or -2, HTLV-1 or -2. A cell population can be generated from a donor that is not positive for anti-hepatitis C (HCV) antibody or HCV NAT. A cell population can be generated from a donor that tests negative for chronic HBV infection. A cell population can be generated from a donor that does not have high potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). A cell population can be generated from a donor that does not have signs or symptoms consistent with active Zika virus infection.

One or more cell populations of the disclosure can be obtained from a single donor, for example, obtained from mobilized peripheral blood apheresis of a single donor. HSPCs, Tregs, Tcons, iNKTs, Tmems, or any combination thereof can be obtained from a single donor.

One or more cell populations of the disclosure can be obtained from one donor, and one or more additional cell populations of the disclosure can be obtained from a second donor. One cell population of the disclosure can be obtained from a single donor, and a second cell population of the disclosure can be obtained from multiple donors. Populations of the disclosure can be obtained from multiple donors, for example, obtained from mobilized peripheral blood apheresis of multiple donors. HSPCs can be obtained from multiple donors. Tregs can be obtained from multiple donors. Tcons can be obtained from multiple donors. iNKTs can be obtained from multiple donors. Tmems can be obtained from multiple donors.

### Doses of Cell Populations

Doses of cell populations administered to a subject (e.g., human subject) may be based on the subject's body weight. In some cases, a subject's body weight may be used to determine a dose of one or more cell populations to be administered to the subject. In some cases, a cell dose may be based on the ideal body weight of the subject instead of their actual weight, e.g., actual body weight. Ideal body weight may be a preferable method of dose calculation to avoid erroneous cell doses due to excess body fat and/or muscle mass. A subject's ideal body weight may be calculated using their height and sex. Other methods that calculate a subject's ideal body weight may be used. For instance, other methods which determine a subject's body fat percentage. A dose of one or more cell populations of the present disclosure, e.g., the first population of CD45⁺ cells, the second population of CD45⁺ cells, and/or the third population of CD45⁺ cells, may be based on the subject's adjusted body weight (ABW), if the subject's actual body weight is greater than 120% of his/her ideal body weight (IBW).

In some embodiments the human subject has a body weight that ranges from approximately 4 kilograms (kg) to approximately 200 kilograms (kg). In some embodiments, the human subject has a body weight that is approximately 4 kilograms, approximately 5 kilograms, approximately 6 kilograms, approximately 7 kilograms, approximately 8 kilograms, approximately 9 kilograms, approximately 10 kilograms, approximately 11 kilograms, approximately 12 kilograms, approximately 13 kilograms, approximately 14 kilograms, approximately 15 kilograms, approximately 16 kilograms, approximately 17 kilograms, approximately 18 kilograms, approximately 19 kilograms, approximately 20 kilograms, approximately 21 kilograms, approximately 22 kilograms, approximately 23 kilograms, approximately 24 kilograms, approximately 25 kilograms, approximately 26 kilograms, approximately 27 kilograms, approximately 28 kilograms, approximately 29 kilograms, approximately 30 kilograms, approximately 31 kilograms, approximately 32 kilograms, approximately 33 kilograms, approximately 34 kilograms, approximately 35 kilograms, approximately 36 kilograms, approximately 37 kilograms, approximately 38 kilograms, approximately 39 kilograms, approximately 40 kilograms, approximately 41 kilograms, approximately 42 kilograms, approximately 43 kilograms, approximately 44 kilograms, approximately 45 kilograms, approximately 46 kilograms, approximately 47 kilograms, approximately 48 kilograms, approximately 49 kilograms, approximately 50 kilograms, approximately 51 kilograms, approximately 52 kilograms, approximately 53 kilograms, approximately 54 kilograms, approximately 55 kilograms, approximately 56 kilograms, approximately 57 kilograms, approximately 58 kilograms, approximately 59 kilograms, approximately 60 kilograms, approximately 61 kilograms, approximately 62 kilograms, approximately 63 kilograms, approximately 64 kilograms, approximately 65 kilograms, approximately 66 kilograms, approximately 67 kilograms, approximately 68 kilograms, approximately 69 kilograms, approximately 70 kilograms, approximately 71 kilograms, approximately 72 kilograms, approximately 73 kilograms, approximately 74 kilograms, approximately 75 kilograms, approximately 76 kilograms, approximately 77 kilograms, approximately 78 kilograms, approximately 79 kilograms, approximately 80 kilograms, approximately 81 kilograms, approximately 82 kilograms, approximately 83 kilograms, approximately 84 kilograms, approximately 85 kilograms, approximately 86 kilograms, approximately 87 kilograms, approximately 88 kilograms, approximately 89 kilograms, approximately 90 kilograms, approximately 91 kilograms, approximately 92 kilograms, approximately 93 kilograms, approximately 94 kilograms, approximately 95 kilograms, approximately 96 kilograms, approximately 97 kilograms, approximately 98 kilograms, approximately 99 kilograms, approximately 100 kilograms, approximately 105 kilograms, approximately 110 kilograms, approximately 115 kilograms, approximately 120 kilograms, approximately 125 kilograms, approximately 130 kilograms, approximately 135 kilograms, approximately 140 kilograms, approximately 145 kilograms, approximately 150 kilograms, approximately 155 kilograms, approximately 160 kilograms, approximately 165 kilograms, approximately 170 kilograms, approximately 175 kilograms, approximately 180 kilograms, approximately 185 kilograms, approximately 190 kilograms, approximately 195 kilograms, or approximately 200 kilograms.

### HSPCs

A first population of CD45⁺ cells which comprises, at least, HSPCs or at least one dose of HSPCs, can comprise at least about 1×10⁴, at least about 2×10⁴, at least about 3×10⁴, at least about 4×10⁴, at least about 5×10⁴, at least about 6×10⁴, at least about 7×10⁴, at least about 8×104, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the first population of CD45⁺ cells and/or HSPCs or doses of HSPCs (e.g., CD34⁺ cells) per kilogram (kg) of recipient subject's (e.g., human subject's) actual body weight or ideal body weight.

A first population of CD45⁺ cells can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the first population of CD45⁺ cells and/or HSPCs or doses of HSPCs (e.g., CD34⁺ cells) per kg of recipient subject's actual body weight or ideal body weight.

For example, a first population of CD45⁺ cells can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the first population of CD45⁺ cells and/or HSPCs or doses of HSPCs (e.g., CD34⁺ cells) per kg of recipient subject's actual body weight or ideal body weight.

In some embodiments, the first population of CD45⁺ cells comprising HSPCs, or comprising at least one dose of HSPCs, comprises from approximately 1.0×10⁵ to approximately 5.0×10¹⁰, from approximately 5.0×10⁵ to approximately 1.5×10¹⁰, from approximately 5.0×10⁵ to approximately 5.0×10⁸, or from approximately 1.5×10⁷ to approximately 1.5×10¹⁰ HSPCs, e.g., in the first population of CD45⁺ cells. In some embodiments, the first population of CD45⁺ cells comprising HSPCs, or comprising at least one dose of HSPCs, comprises approximately 1.0×10⁵ or more HSPCs, approximately 2.0×10⁵ or more HSPCs, approximately 3.0×10⁵ or more HSPCs, approximately 4.0×10⁵ or more HSPCs, approximately 5.0×10⁵ or more HSPCs, approximately 6.0×10⁵ or more HSPCs, approximately 7.0×10⁵ or more HSPCs, approximately 8.0×10⁵ or more HSPCs, approximately 9.0×10⁵ or more HSPCs, approximately 1.0×10⁶ or more HSPCs, approximately 1.0×10⁶ or more HSPCs, approximately 1.1×10⁶ or more HSPCs, approximately 1.2×10⁶ or more HSPCs, approximately 1.3×10⁶ or more HSPCs, approximately 1.4×10⁶ or more HSPCs, approximately 1.5×10⁶ or more HSPCs, approximately 1.6×10⁶ or more HSPCs, approximately 1.7×10⁶ or more HSPCs, approximately 1.8×10⁶ or more HSPCs, approximately 1.9×10⁶ or more HSPCs, approximately 2.0×10⁶ or more HSPCs, approximately 2.1×10⁶ or more HSPCs, approximately 2.2×10⁶ or more HSPCs, approximately 2.3×10⁶ or more HSPCs, approximately 2.4×10⁶ or more HSPCs, approximately 2.5×10⁶ or more HSPCs, approximately 2.6×10⁶ or more HSPCs, approximately 2.7×10⁶ or more HSPCs, approximately 2.8×10⁶ or more HSPCs, approximately 2.9×10⁶ or more HSPCs, approximately 3.0×10⁶ or more HSPCs, approximately 3.1×10⁶ or more HSPCs, approximately 3.2×10⁶ or more HSPCs, approximately 3.3×10⁶ or more HSPCs, approximately 3.4×10⁶ or more HSPCs, approximately 3.5×10⁶ or more HSPCs, approximately 3.6×10⁶ or more HSPCs, approximately 3.7×10⁶ or more HSPCs, approximately 3.8×10⁶ or more HSPCs, approximately 3.9×10⁶ or more HSPCs, approximately 4.0×10⁶ or more HSPCs, approximately 4.1×10⁶ or more HSPCs, approximately 4.2×10⁶ or more HSPCs, approximately 4.3×10⁶ or more HSPCs, approximately 4.4×10⁶ or more HSPCs, approximately 4.5×10⁶ or more HSPCs, approximately 4.6×10⁶ or more HSPCs, approximately 4.7×10⁶ or more HSPCs, approximately 4.8×10⁶ or more HSPCs, approximately 4.9×10⁶ or more HSPCs, approximately 5.0×10⁶ or more HSPCs, approximately 5.1×10⁶ or more HSPCs, approximately 5.2×10⁶ or more HSPCs, approximately 5.3×10⁶ or more HSPCs, approximately 5.4×10⁶ or more HSPCs, approximately 5.5×10⁶ or more HSPCs, approximately 5.6×10⁶ or more HSPCs, approximately 5.7×10⁶ or more HSPCs, approximately 5.8×10⁶ or more HSPCs, approximately 5.9×10⁶ or more HSPCs, approximately 6.0×10⁶ or more HSPCs, approximately 6.1×10⁶ or more HSPCs, approximately 6.2×10⁶ or more HSPCs, approximately 6.3×10⁶ or more HSPCs, approximately 6.4×10⁶ or more HSPCs, approximately 6.5×10⁶ or more HSPCs, approximately 6.6×10⁶ or more HSPCs, approximately 6.7×10⁶ or more HSPCs, approximately 6.8×10⁶ or more HSPCs, approximately 6.9×10⁶ or more HSPCs, approximately 7.0×10⁶ or more HSPCs, approximately 7.1×10⁶ or more HSPCs, approximately 7.2×10⁶ or more HSPCs, approximately 7.3×10⁶ or more HSPCs, approximately 7.4×10⁶ or more HSPCs, approximately 7.5×10⁶ or more HSPCs, approximately 7.6×10⁶ or more HSPCs, approximately 7.7×10⁶ or more HSPCs, approximately 7.8×10⁶ or more HSPCs, approximately 7.9×10⁶ or more HSPCs, approximately 8.0×10⁶ or more HSPCs, approximately 8.1×10⁶ or more HSPCs, approximately 8.2×10⁶ or more HSPCs, approximately 8.3×10⁶ or more HSPCs, approximately 8.4×10⁶ or more HSPCs, approximately 8.5×10⁶ or more HSPCs, approximately 8.6×10⁶ or more HSPCs, approximately 8.7×10⁶ or more HSPCs, approximately 8.8×10⁶ or more HSPCs, approximately 8.9×10⁶ or more HSPCs, approximately 9.0×10⁶ or more HSPCs, approximately 9.1×10⁶ or more HSPCs, approximately 9.2×10⁶ or more HSPCs, approximately 9.3×10⁶ or more HSPCs, approximately 9.4×10⁶ or more HSPCs, approximately 9.5×10⁶ or more HSPCs, approximately 9.6×10⁶ or more HSPCs, approximately 9.7×10⁶ or more HSPCs, approximately 9.8×10⁶ or more HSPCs, approximately 9.9×10⁶ or more HSPCs, approximately 1.0×10⁷ or more HSPCs, approximately 1.1×10⁷ or more HSPCs, approximately 1.2×10⁷ or more HSPCs, approximately 1.3×10⁷ or more HSPCs, approximately 1.4×10⁷ or more HSPCs, approximately 1.5×10⁷ or more HSPCs, approximately 1.6×10⁷ or more HSPCs, approximately 1.7×10⁷ or more HSPCs, approximately 1.8×10⁷ or more HSPCs, approximately 1.9×10⁷ or more HSPCs, approximately 2.0×10⁷ or more HSPCs, approximately 2.1×10⁷ or more HSPCs, approximately 2.2×10⁷ or more HSPCs, approximately 2.3×10⁷ or more HSPCs, approximately 2.4×10⁷ or more HSPCs, approximately 2.5×10⁷ or more HSPCs, approximately 2.6×10⁷ or more HSPCs, approximately 2.7×10⁷ or more HSPCs, approximately 2.8×10⁷ or more HSPCs, approximately 2.9×10⁷ or more HSPCs, approximately 3.0×10⁷ or more HSPCs, approximately 3.1×10⁷ or more HSPCs, approximately 3.2×10⁷ or more HSPCs, approximately 3.3×10⁷ or more HSPCs, approximately 3.4×10⁷ or more HSPCs, approximately 3.5×10⁷ or more HSPCs, approximately 3.6×10⁷ or more HSPCs, approximately 3.7×10⁷ or more HSPCs, approximately 3.8×10⁷ or more HSPCs, approximately 3.9×10⁷ or more HSPCs, approximately 4.0×10⁷ or more HSPCs, approximately 4.1×10⁷ or more HSPCs, approximately 4.2×10⁷ or more HSPCs, approximately 4.3×10⁷ or more HSPCs, approximately 4.4×10⁷ or more HSPCs, approximately 4.5×10⁷ or more HSPCs, approximately 4.6×10⁷ or more HSPCs, approximately 4.7×10⁷ or more HSPCs, approximately 4.8×10⁷ or more HSPCs, approximately 4.9×10⁷ or more HSPCs, approximately 5.0×10⁷ or more HSPCs, approximately 5.1×10⁷ or more HSPCs, approximately 5.2×10⁷ or more HSPCs, approximately 5.3×10⁷ or more HSPCs, approximately 5.4×10⁷ or more HSPCs, approximately 5.5×10⁷ or more HSPCs, approximately 5.6×10⁷ or more HSPCs, approximately 5.7×10⁷ or more HSPCs, approximately 5.8×10⁷ or more HSPCs, approximately 5.9×10⁷ or more HSPCs, approximately 6.0×10⁷ or more HSPCs, approximately 6.1×10⁷ or more HSPCs, approximately 6.2×10⁷ or more HSPCs, approximately 6.3×10⁷ or more HSPCs, approximately 6.4×10⁷ or more HSPCs, approximately 6.5×10⁷ or more HSPCs, approximately 6.6×10⁷ or more HSPCs, approximately 6.7×10⁷ or more HSPCs, approximately 6.8×10⁷ or more HSPCs, approximately 6.9×10⁷ or more HSPCs, approximately 7.0×10⁷ or more HSPCs, approximately 7.1×10⁷ or more HSPCs, approximately 7.2×10⁷ or more HSPCs, approximately 7.3×10⁷ or more HSPCs, approximately 7.4×10⁷ or more HSPCs, approximately 7.5×10⁷ or more HSPCs, approximately 7.6×10⁷ or more HSPCs, approximately 7.7×10⁷ or more HSPCs, approximately 7.8×10⁷ or more HSPCs, approximately 7.9×10⁷ or more HSPCs, approximately 8.0×10⁷ or more HSPCs, approximately 8.1×10⁷ or more HSPCs, approximately 8.2×10⁷ or more HSPCs, approximately 8.3×10⁷ or more HSPCs, approximately 8.4×10⁷ or more HSPCs, approximately 8.5×10⁷ or more HSPCs, approximately 8.6×10⁷ or more HSPCs, approximately 8.7×10⁷ or more HSPCs, approximately 8.8×10⁷ or more HSPCs, approximately 8.9×10⁷ or more HSPCs, approximately 9.0×10⁷ or more HSPCs, approximately 9.1×10⁷ or more HSPCs, approximately 9.2×10⁷ or more HSPCs, approximately 9.3×10⁷ or more HSPCs, approximately 9.4×10⁷ or more HSPCs, approximately 9.5×10⁷ or more HSPCs, approximately 9.6×10⁷ or more HSPCs, approximately 9.7×10⁷ or more HSPCs, approximately 9.8×10⁷ or more HSPCs, approximately 9.9×10⁷ or more HSPCs, approximately 1.0×10⁸ or more HSPCs, approximately 1.1×10⁸ or more HSPCs, approximately 1.2×10⁸ or more HSPCs, approximately 1.3×10⁸ or more HSPCs, approximately 1.4×10⁸ or more HSPCs, approximately 1.5×10⁸ or more HSPCs, approximately 1.6×10⁸ or more HSPCs, approximately 1.7×10⁸ or more HSPCs, approximately 1.8×10⁸ or more HSPCs, approximately 1.9×10⁸ or more HSPCs, approximately 2.0×10⁸ or more HSPCs, approximately 2.1×10⁸ or more HSPCs, approximately 2.2×10⁸ or more HSPCs, approximately 2.3×10⁸ or more HSPCs, approximately 2.4×10⁸ or more HSPCs, approximately 2.5×10⁸ or more HSPCs, approximately 2.6×10⁸ or more HSPCs, approximately 2.7×10⁸ or more HSPCs, approximately 2.8×10⁸ or more HSPCs, approximately 2.9×10⁸ or more HSPCs, approximately 3.0×10⁸ or more HSPCs, approximately 3.1×10⁸ or more HSPCs, approximately 3.2×10⁸ or more HSPCs, approximately 3.3×10⁸ or more HSPCs, approximately 3.4×10⁸ or more HSPCs, approximately 3.5×10⁸ or more HSPCs, approximately 3.6×10⁸ or more HSPCs, approximately 3.7×10⁸ or more HSPCs, approximately 3.8×10⁸ or more HSPCs, approximately 3.9×10⁸ or more HSPCs, approximately 4.0×10⁸ or more HSPCs, approximately 4.1×10⁸ or more HSPCs, approximately 4.2×10⁸ or more HSPCs, approximately 4.3×10⁸ or more HSPCs, approximately 4.4×10⁸ or more HSPCs, approximately 4.5×10⁸ or more HSPCs, approximately 4.6×10⁸ or more HSPCs, approximately 4.7×10⁸ or more HSPCs, approximately 4.8×10⁸ or more HSPCs, approximately 4.9×10⁸ or more HSPCs, approximately 5.0×10⁸ or more HSPCs, approximately 5.1×10⁸ or more HSPCs, approximately 5.2×10⁸ or more HSPCs, approximately 5.3×10⁸ or more HSPCs, approximately 5.4×10⁸ or more HSPCs, approximately 5.5×10⁸ or more HSPCs, approximately 5.6×10⁸ or more HSPCs, approximately 5.7×10⁸ or more HSPCs, approximately 5.8×10⁸ or more HSPCs, approximately 5.9×10⁸ or more HSPCs, approximately 6.0×10⁸ or more HSPCs, approximately 6.1×10⁸ or more HSPCs, approximately 6.2×10⁸ or more HSPCs, approximately 6.3×10⁸ or more HSPCs, approximately 6.4×10⁸ or more HSPCs, approximately 6.5×10⁸ or more HSPCs, approximately 6.6×10⁸ or more HSPCs, approximately 6.7×10⁸ or more HSPCs, approximately 6.8×10⁸ or more HSPCs, approximately 6.9×10⁸ or more HSPCs, approximately 7.0×10⁸ or more HSPCs, approximately 7.1×10⁸ or more HSPCs, approximately 7.2×10⁸ or more HSPCs, approximately 7.3×10⁸ or more HSPCs, approximately 7.4×10⁸ or more HSPCs, approximately 7.5×10⁸ or more HSPCs, approximately 7.6×10⁸ or more HSPCs, approximately 7.7×10⁸ or more HSPCs, approximately 7.8×10⁸ or more HSPCs, approximately 7.9×10⁸ or more HSPCs, approximately 8.0×10⁸ or more HSPCs, approximately 8.1×10⁸ or more HSPCs, approximately 8.2×10⁸ or more HSPCs, approximately 8.3×10⁸ or more HSPCs, approximately 8.4×10⁸ or more HSPCs, approximately 8.5×10⁸ or more HSPCs, approximately 8.6×10⁸ or more HSPCs, approximately 8.7×10⁸ or more HSPCs, approximately 8.8×10⁸ or more HSPCs, approximately 8.9×10⁸ or more HSPCs, approximately 9.0×10⁸ or more HSPCs, approximately 9.1×10⁸ or more HSPCs, approximately 9.2×10⁸ or more HSPCs, approximately 9.3×10⁸ or more HSPCs, approximately 9.4×10⁸ or more HSPCs, approximately 9.5×10⁸ or more HSPCs, approximately 9.6×10⁸ or more HSPCs, approximately 9.7×10⁸ or more HSPCs, approximately 9.8×10⁸ or more HSPCs, approximately 9.9×10⁸ or more HSPCs, approximately 1.0×10⁹ or more HSPCs, approximately 1.1×10⁹ or more HSPCs, approximately 1.2×10⁹ or more HSPCs, approximately 1.3×10⁹ or more HSPCs, approximately 1.4×10⁹ or more HSPCs, approximately 1.5×10⁹ or more HSPCs, approximately 1.6×10⁹ or more HSPCs, approximately 1.7×10⁹ or more HSPCs, approximately 1.8×10⁹ or more HSPCs, approximately 1.9×10⁹ or more HSPCs, approximately 2.0×10⁹ or more HSPCs, approximately 2.1×10⁹ or more HSPCs, approximately 2.2×10⁹ or more HSPCs, approximately 2.3×10⁹ or more HSPCs, approximately 2.4×10⁹ or more HSPCs, approximately 2.5×10⁹ or more HSPCs, approximately 2.6×10⁹ or more HSPCs, approximately 2.7×10⁹ or more HSPCs, approximately 2.8×10⁹ or more HSPCs, approximately 2.9×10⁹ or more HSPCs, approximately 3.0×10⁹ or more HSPCs, approximately 3.1×10⁹ or more HSPCs, approximately 3.2×10⁹ or more HSPCs, approximately 3.3×10⁹ or more HSPCs, approximately 3.4×10⁹ or more HSPCs, approximately 3.5×10⁹ or more HSPCs, approximately 3.6×10⁹ or more HSPCs, approximately 3.7×10⁹ or more HSPCs, approximately 3.8×10⁹ or more HSPCs, approximately 3.9×10⁹ or more HSPCs, approximately 4.0×10⁹ or more HSPCs, approximately 4.1×10⁹ or more HSPCs, approximately 4.2×10⁹ or more HSPCs, approximately 4.3×10⁹ or more HSPCs, approximately 4.4×10⁹ or more HSPCs, approximately 4.5×10⁹ or more HSPCs, approximately 4.6×10⁹ or more HSPCs, approximately 4.7×10⁹ or more HSPCs, approximately 4.8×10⁹ or more HSPCs, approximately 4.9×10⁹ or more HSPCs, approximately 5.0×10⁹ or more HSPCs, approximately 5.1×10⁹ or more HSPCs, approximately 5.2×10⁹ or more HSPCs, approximately 5.3×10⁹ or more HSPCs, approximately 5.4×10⁹ or more HSPCs, approximately 5.5×10⁹ or more HSPCs, approximately 5.6×10⁹ or more HSPCs, approximately 5.7×10⁹ or more HSPCs, approximately 5.8×10⁹ or more HSPCs, approximately 5.9×10⁹ or more HSPCs, approximately 6.0×10⁹ or more HSPCs, approximately 6.1×10⁹ or more HSPCs, approximately 6.2×10⁹ or more HSPCs, approximately 6.3×10⁹ or more HSPCs, approximately 6.4×10⁹ or more HSPCs, approximately 6.5×10⁹ or more HSPCs, approximately 6.6×10⁹ or more HSPCs, approximately 6.7×10⁹ or more HSPCs, approximately 6.8×10⁹ or more HSPCs, approximately 6.9×10⁹ or more HSPCs, approximately 7.0×10⁹ or more HSPCs, approximately 7.1×10⁹ or more HSPCs, approximately 7.2×10⁹ or more HSPCs, approximately 7.3×10⁹ or more HSPCs, approximately 7.4×10⁹ or more HSPCs, approximately 7.5×10⁹ or more HSPCs, approximately 7.6×10⁹ or more HSPCs, approximately 7.7×10⁹ or more HSPCs, approximately 7.8×10⁹ or more HSPCs, approximately 7.9×10⁹ or more HSPCs, approximately 8.0×10⁹ or more HSPCs, approximately 8.1×10⁹ or more HSPCs, approximately 8.2×10⁹ or more HSPCs, approximately 8.3×10⁹ or more HSPCs, approximately 8.4×10⁹ or more HSPCs, approximately 8.5×10⁹ or more HSPCs, approximately 8.6×10⁹ or more HSPCs, approximately 8.7×10⁹ or more HSPCs, approximately 8.8×10⁹ or more HSPCs, approximately 8.9×10⁹ or more HSPCs, approximately 9.0×10⁹ or more HSPCs, approximately 9.1×10⁹ or more HSPCs, approximately 9.2×10⁹ or more HSPCs, approximately 9.3×10⁹ or more HSPCs, approximately 9.4×10⁹ or more HSPCs, approximately 9.5×10⁹ or more HSPCs, approximately 9.6×10⁹ or more HSPCs, approximately 9.7×10⁹ or more HSPCs, approximately 9.8×10⁹ or more HSPCs, approximately 9.9×10⁹ or more HSPCs, approximately 1.0×10¹⁰ or more HSPCs, approximately 1.1×10¹⁰ or more HSPCs, approximately 1.2×10¹⁰ or more HSPCs, approximately 1.3×10¹⁰ or more HSPCs, approximately 1.4×10¹⁰ or more HSPCs, approximately 1.5×10¹⁰ or more HSPCs, approximately 1.6×10¹⁰ or more HSPCs, approximately 1.7×10¹⁰ or more HSPCs, approximately 1.8×10¹⁰ or more HSPCs, approximately 1.9×10¹⁰ or more HSPCs, approximately 2.0×10¹⁰ or more HSPCs, approximately 2.1×10¹⁰ or more HSPCs, approximately 2.2×10¹⁰ or more HSPCs, approximately 2.3×10¹⁰ or more HSPCs, approximately 2.4×10¹⁰ or more HSPCs, approximately 2.5×10¹⁰ or more HSPCs, approximately 2.6×10¹⁰ or more HSPCs, approximately 2.7×10¹⁰ or more HSPCs, approximately 2.8×10¹⁰ or more HSPCs, approximately 2.9×10¹⁰ or more HSPCs, approximately 3.0×10¹⁰ or more HSPCs, approximately 3.1×10¹⁰ or more HSPCs, approximately 3.2×10¹⁰ or more HSPCs, approximately 3.3×10¹⁰ or more HSPCs, approximately 3.4×10¹⁰ or more HSPCs, approximately 3.5×10¹⁰ or more HSPCs, approximately 3.6×10¹⁰ or more HSPCs, approximately 3.7×10¹⁰ or more HSPCs, approximately 3.8×10¹⁰ or more HSPCs, approximately 3.9×10¹⁰ or more HSPCs, approximately 4.0×10¹⁰ or more HSPCs, approximately 4.1×10¹⁰ or more HSPCs, approximately 4.2×10¹⁰ or more HSPCs, approximately 4.3×10¹⁰ or more HSPCs, approximately 4.4×10¹⁰ or more HSPCs, approximately 4.5×10¹⁰ or more HSPCs, approximately 4.6×10¹⁰ or more HSPCs, approximately 4.7×10¹⁰ or more HSPCs, approximately 4.8×110¹⁰ or more HSPCs, approximately 4.9×10¹⁰ or more HSPCs, or approximately 5.0×10¹⁰ or more HSPCs.

A first population of CD45⁺ cells can have a defined level of purity for CD34⁺ cells. For example, a first population of CD45⁺ cells can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more CD34⁺ cells as a percentage of total cells, as a percentage of nucleated cells, or as a percentage of CD45⁺ cells.

A first population of CD45⁺ cells can have a defined level of contaminating CD3⁺ cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ CD3⁺ cells per kg of a recipient subject's body weight are present in a first population of CD45⁺ cells or ideal body weight.

In some embodiments, a first population of CD45⁺ cells comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% CD3⁺ cells as a percentage of total cells, as a percentage of nucleated cells, or as a percentage of CD45⁺ cells.

In some embodiments, the first population of CD45⁺ cells comprises less than about 5 EU of endotoxins per ml of the solution, less than about 1 EU of endotoxins per ml of the solution, and/or less than about 0.5 EU of endotoxins per ml of the solution.

In embodiments, at least one of the cell populations comprise less than about 5 EU of endotoxins/ml of respective suspension liquid.

A first population of CD45⁺ cells can comprise 0.5 EU/ml endotoxins to 10 EU/ml endotoxins. A first population of CD45⁺ cells can comprise at least 0.5 EU/ml endotoxins. A first population of CD45⁺ cells can comprise at most 10 EU/ml endotoxins. A first population of CD45⁺ cells can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A first population of CD45⁺ cells can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A first population of CD45⁺ cells can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A first population of CD45⁺ cells can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A first population of CD45⁺ cells can comprise 0.5% w/w to 10% w/w unbound reagents. These unbound reagents may include any affinity reagents used for the sorting of HSPCs, for instance, antibodies, or purification particles or magnetic particles. A first population of CD45⁺ cells can comprise at least 0.5% w/w unbound reagents. A first population of CD45⁺ cells can comprise at most 10% w/w unbound reagents. A first population of CD45⁺ cells can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A first population of CD45⁺ cells can comprise 10% w/w, 8% w/w, 6% w/w, 5% w/w, 4% w/w, 2% w/w, 1% w/w, or 0.5% w/w unbound reagents. A first population of CD45⁺ cells can comprise at least 8% w/w, 6% w/w, 5% w/w, 4% w/w, 2% w/w, 1% w/w, or 0.5% w/w unbound reagents. A first population of CD45⁺ cells can comprise at most 10% w/w, 8% w/w, 6% w/w, 5% w/w, 4% w/w, 2% w/w or 1% w/w unbound reagents.

A first population of CD45⁺ cells can comprise 5×10³ to 90×10³ microbeads per cell. These microbeads may comprise microbeads used to purify the HSPC population, for instance, an anti-CD34 antibody comprising microbead used to sort the HSPC population. A first population of CD45⁺ cells can comprise at least 5×10³ microbeads per cell. A first population of CD45⁺ cells can comprise at most 90×10³ microbeads per cell. A first population of CD45⁺ cells can comprise 90×10³ to 70×10³, 90×10³ to 50×10³, 90×10³ to 40×10³, 90×10³ to 30×10³, 90×10³ to 20×10³, 90×10³ to 10×10³, 90×10³ to 5×10³, 70×10³ to 50×10³, 70×10³ to 40×10³, 70×10³ to 30×10³, 70×10³ to 20×10³, 70×10³ to 10×10³, 70×10³ to 5×10³, 50×10³ to 40×10³, 50×10³ to 30×10³, 50×10³ to 20×10³, 50×10³ to 10×10³, 50×10³ to 5×10³, 40×10³ to 30×10³, 40×10³ to 20×10³, 40×10³ to 10×10³, 40×10³ to 5×10³, 30×10³ to 20×10³, 30×10³ to 10×10³, 30×10³ to 5×10³, 20×10³ to 10×10³, 20×10³ to 5×10³, or 10×10³ to 5×10³ microbeads per cell. A first population of CD45⁺ cells can comprise 90×10³, 70×10³, 50×10³, 40×10³, 30×10³, 20×10³, 10×10³, or 5×10³ microbeads per cell. A first population of CD45⁺ cells can comprise at least 70×10³, 50×10³, 40×10³, 30×10³, 20×10³, 10×10³, or 5×10³ microbeads per cell. A first population of CD45⁺ cells can comprise at most 90×10³, 70×10³, 50×10³, 40×10³, 30×10³, 20×10³, or 10×10³ microbeads per cell.

### Tregs

A population of cells enriched for Tregs or comprising at least one dose of Tregs, e.g., a second population of CD45⁺ cells comprising at least one dose of Tregs, can comprise at least about 1×10⁴, at least about 1×10⁵, at least about 2×10⁴, at least about 3×10⁴, at least about 4×10⁴, at least about 5×10⁴, at least about 6×10⁴, at least about 7×10⁴, at least about 8×10⁴, least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×107, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×108, at least about 7.5×10⁸, at least about 8×10⁸, at least about 8.5×10⁸, at least about 9×10⁸, at least about 9.5×10⁸, at least about 1×10⁹, or more cells of the cell population enriched for Tregs (e.g., the second population of CD45⁺ cells) and/or Tregs or doses of Tregs per kg of recipient subject's actual body weight or ideal body weight (e.g., where Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim}FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺ CD127^{dim}).

A population of cells enriched for Tregs or comprising at least one dose of Tregs, e.g., a second population of CD45⁺ cells comprising at least one dose of Tregs, can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the cell population enriched for Tregs (e.g., the second population of CD45⁺ cells) and/or Tregs or doses of Tregs per kg of recipient subject's actual body weight or ideal body weight (e.g., where Tregs are CD4⁺ CD25⁺ CD127^{dim}, CD3⁺CD4⁺CD25⁺, CD3⁺CD4⁺CD25⁺CD127^{dim}, CD3⁺CD4⁺CD25⁺CD127^{dim}FOXP3⁺, CD3⁺FOXP3⁺, CD3⁺CD4⁺FOXP3⁺, CD3⁺CD4⁺CD25⁺FOXP3⁺, CD3⁺CD25⁺FOXP3+, CD3⁺CD25⁺CD127^{dim}, CD4⁺ CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺ CD127^{dim}).

For example, a population of cells enriched for Tregs or comprising at least one dose of Tregs, e.g., a second population of CD45⁺ cells comprising at least one dose of Tregs, can comprise 1×10⁴ to 1×10⁹, 1×10⁵ to 1×10⁸, 1×10⁵ to 2×10⁷, 5×10⁵ to 2×10⁷, 5×10⁵ to 1.5×10₇, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the cell population enriched for Tregs (e.g., the second population of CD45⁺ cells) and/or Tregs or doses of Tregs per kg of recipient subject's actual body weight or ideal body weight (e.g., where Tregs are CD4⁺CD25⁺CD127^{dim}, CD3⁺CD4⁺CD25⁺, CD3⁺CD4⁺CD25⁺ CD127^{dim}, CD3⁺CD4⁺CD25⁺CD127^{dim}FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺ CD127^{dim}).

In some embodiments, the population of cells enriched for Tregs or comprising at least one dose of Tregs, e.g., a second population of CD45⁺ cells comprising at least one dose of Tregs, comprises from approximately 1.0×10⁵ to approximately 5.0×10⁹, from approximately 5.0×10⁵ to approximately 3.0×10⁹, from approximately 1.5×10⁷ to approximately 3.0×10⁹, or from approximately 5.0×10⁵ to approximately 1.0×10⁸ Tregs, e.g., fresh isolated Tregs in the population of cells enriched for Tregs. In some embodiments, the population of cells enriched for Tregs or comprising at least one dose of Tregs, e.g., a second population of CD45⁺ cells comprising at least one dose of Tregs, comprises approximately 1.0×10⁵ or more Tregs, approximately 2.0×10⁵ or more Tregs, approximately 3.0×10⁵ or more Tregs, approximately 4.0×10⁵ or more Tregs, approximately 5.0×10⁵ or more Tregs, approximately 6.0×10⁵ or more Tregs, approximately 7.0×10⁵ or more Tregs, approximately 8.0×10⁵ or more Tregs, approximately 9.0×10⁵ or more Tregs, approximately 1.0×10⁶ or more Tregs, approximately 1.0×10⁶ or more Tregs, approximately 1.1×10⁶ or more Tregs, approximately 1.2×10⁶ or more Tregs, approximately 1.3×10⁶ or more Tregs, approximately 1.4×10⁶ or more Tregs, approximately 1.5×10⁶ or more Tregs, approximately 1.6×10⁶ or more Tregs, approximately 1.7×10⁶ or more Tregs, approximately 1.8×10⁶ or more Tregs, approximately 1.9×10⁶ or more Tregs, approximately 2.0×10⁶ or more Tregs, approximately 2.1×10⁶ or more Tregs, approximately 2.2×10⁶ or more Tregs, approximately 2.3×10⁶ or more Tregs, approximately 2.4×10⁶ or more Tregs, approximately 2.5×10⁶ or more Tregs, approximately 2.6×10⁶ or more Tregs, approximately 2.7×10⁶ or more Tregs, approximately 2.8×10⁶ or more Tregs, approximately 2.9×10⁶ or more Tregs, approximately 3.0×10⁶ or more Tregs, approximately 3.1×10⁶ or more Tregs, approximately 3.2×10⁶ or more Tregs, approximately 3.3×10⁶ or more Tregs, approximately 3.4×10⁶ or more Tregs, approximately 3.5×10⁶ or more Tregs, approximately 3.6×10⁶ or more Tregs, approximately 3.7×10⁶ or more Tregs, approximately 3.8×10⁶ or more Tregs, approximately 3.9×10⁶ or more Tregs, approximately 4.0×10⁶ or more Tregs, approximately 4.1×10⁶ or more Tregs, approximately 4.2×10⁶ or more Tregs, approximately 4.3×10⁶ or more Tregs, approximately 4.4×10⁶ or more Tregs, approximately 4.5×10⁶ or more Tregs, approximately 4.6×10⁶ or more Tregs, approximately 4.7×10⁶ or more Tregs, approximately 4.8×10⁶ or more Tregs, approximately 4.9×10⁶ or more Tregs, approximately 5.0×10⁶ or more Tregs, approximately 5.1×10⁶ or more Tregs, approximately 5.2×10⁶ or more Tregs, approximately 5.3×10⁶ or more Tregs, approximately 5.4×10⁶ or more Tregs, approximately 5.5×10⁶ or more Tregs, approximately 5.6×10⁶ or more Tregs, approximately 5.7×10⁶ or more Tregs, approximately 5.8×10⁶ or more Tregs, approximately 5.9×10⁶ or more Tregs, approximately 6.0×10⁶ or more Tregs, approximately 6.1×10⁶ or more Tregs, approximately 6.2×10⁶ or more Tregs, approximately 6.3×10⁶ or more Tregs, approximately 6.4×10⁶ or more Tregs, approximately 6.5×10⁶ or more Tregs, approximately 6.6×10⁶ or more Tregs, approximately 6.7×10⁶ or more Tregs, approximately 6.8×10⁶ or more Tregs, approximately 6.9×10⁶ or more Tregs, approximately 7.0×10⁶ or more Tregs, approximately 7.1×10⁶ or more Tregs, approximately 7.2×10⁶ or more Tregs, approximately 7.3×10⁶ or more Tregs, approximately 7.4×10⁶ or more Tregs, approximately 7.5×10⁶ or more Tregs, approximately 7.6×10⁶ or more Tregs, approximately 7.7×10⁶ or more Tregs, approximately 7.8×10⁶ or more Tregs, approximately 7.9×10⁶ or more Tregs, approximately 8.0×10⁶ or more Tregs, approximately 8.1×10⁶ or more Tregs, approximately 8.2×10⁶ or more Tregs, approximately 8.3×10⁶ or more Tregs, approximately 8.4×10⁶ or more Tregs, approximately 8.5×10⁶ or more Tregs, approximately 8.6×10⁶ or more Tregs, approximately 8.7×10⁶ or more Tregs, approximately 8.8×10⁶ or more Tregs, approximately 8.9×10⁶ or more Tregs, approximately 9.0×10⁶ or more Tregs, approximately 9.1×10⁶ or more Tregs, approximately 9.2×10⁶ or more Tregs, approximately 9.3×10⁶ or more Tregs, approximately 9.4×10⁶ or more Tregs, approximately 9.5×10⁶ or more Tregs, approximately 9.6×10⁶ or more Tregs, approximately 9.7×10⁶ or more Tregs, approximately 9.8×10⁶ or more Tregs, approximately 9.9×10⁶ or more Tregs, approximately 1.0×10⁷ or more Tregs, approximately 1.1×10⁷ or more Tregs, approximately 1.2×10⁷ or more Tregs, approximately 1.3×10⁷ or more Tregs, approximately 1.4×10⁷ or more Tregs, approximately 1.5×10⁷ or more Tregs, approximately 1.6×10⁷ or more Tregs, approximately 1.7×10⁷ or more Tregs, approximately 1.8×10⁷ or more Tregs, approximately 1.9×10⁷ or more Tregs, approximately 2.0×10⁷ or more Tregs, approximately 2.1×10⁷ or more Tregs, approximately 2.2×10⁷ or more Tregs, approximately 2.3×10⁷ or more Tregs, approximately 2.4×10⁷ or more Tregs, approximately 2.5×10⁷ or more Tregs, approximately 2.6×10⁷ or more Tregs, approximately 2.7×10⁷ or more Tregs, approximately 2.8×10⁷ or more Tregs, approximately 2.9×10⁷ or more Tregs, approximately 3.0×10⁷ or more Tregs, approximately 3.1×10⁷ or more Tregs, approximately 3.2×10⁷ or more Tregs, approximately 3.3×10⁷ or more Tregs, approximately 3.4×10⁷ or more Tregs, approximately 3.5×10⁷ or more Tregs, approximately 3.6×10⁷ or more Tregs, approximately 3.7×10⁷ or more Tregs, approximately 3.8×10⁷ or more Tregs, approximately 3.9×10⁷ or more Tregs, approximately 4.0×10⁷ or more Tregs, approximately 4.1×10⁷ or more Tregs, approximately 4.2×10⁷ or more Tregs, approximately 4.3×10⁷ or more Tregs, approximately 4.4×10⁷ or more Tregs, approximately 4.5×10⁷ or more Tregs, approximately 4.6×10⁷ or more Tregs, approximately 4.7×10⁷ or more Tregs, approximately 4.8×10⁷ or more Tregs, approximately 4.9×10⁷ or more Tregs, approximately 5.0×10⁷ or more Tregs, approximately 5.1×10⁷ or more Tregs, approximately 5.2×10⁷ or more Tregs, approximately 5.3×10⁷ or more Tregs, approximately 5.4×10⁷ or more Tregs, approximately 5.5×10⁷ or more Tregs, approximately 5.6×10⁷ or more Tregs, approximately 5.7×10⁷ or more Tregs, approximately 5.8×10⁷ or more Tregs, approximately 5.9×10⁷ or more Tregs, approximately 6.0×10⁷ or more Tregs, approximately 6.1×10⁷ or more Tregs, approximately 6.2×10⁷ or more Tregs, approximately 6.3×10⁷ or more Tregs, approximately 6.4×10⁷ or more Tregs, approximately 6.5×10⁷ or more Tregs, approximately 6.6×10⁷ or more Tregs, approximately 6.7×10⁷ or more Tregs, approximately 6.8×10⁷ or more Tregs, approximately 6.9×10⁷ or more Tregs, approximately 7.0×10⁷ or more Tregs, approximately 7.1×10⁷ or more Tregs, approximately 7.2×10⁷ or more Tregs, approximately 7.3×10⁷ or more Tregs, approximately 7.4×10⁷ or more Tregs, approximately 7.5×10⁷ or more Tregs, approximately 7.6×10⁷ or more Tregs, approximately 7.7×10⁷ or more Tregs, approximately 7.8×10⁷ or more Tregs, approximately 7.9×10⁷ or more Tregs, approximately 8.0×10⁷ or more Tregs, approximately 8.1×10⁷ or more Tregs, approximately 8.2×10⁷ or more Tregs, approximately 8.3×10⁷ or more Tregs, approximately 8.4×10⁷ or more Tregs, approximately 8.5×10⁷ or more Tregs, approximately 8.6×10⁷ or more Tregs, approximately 8.7×10⁷ or more Tregs, approximately 8.8×10⁷ or more Tregs, approximately 8.9×10⁷ or more Tregs, approximately 9.0×10⁷ or more Tregs, approximately 9.1×10⁷ or more Tregs, approximately 9.2×10⁷ or more Tregs, approximately 9.3×10⁷ or more Tregs, approximately 9.4×10⁷ or more Tregs, approximately 9.5×10⁷ or more Tregs, approximately 9.6×10⁷ or more Tregs, approximately 9.7×10⁷ or more Tregs, approximately 9.8×10⁷ or more Tregs, approximately 9.9×10⁷ or more Tregs, approximately 1.0×10⁸ or more Tregs, approximately 1.1×10⁸ or more Tregs, approximately 1.2×10⁸ or more Tregs, approximately 1.3×10⁸ or more Tregs, approximately 1.4×10⁸ or more Tregs, approximately 1.5×10⁸ or more Tregs, approximately 1.6×10⁸ or more Tregs, approximately 1.7×10⁸ or more Tregs, approximately 1.8×10⁸ or more Tregs, approximately 1.9×10⁸ or more Tregs, approximately 2.0×10⁸ or more Tregs, approximately 2.1×10⁸ or more Tregs, approximately 2.2×10⁸ or more Tregs, approximately 2.3×10⁸ or more Tregs, approximately 2.4×10⁸ or more Tregs, approximately 2.5×10⁸ or more Tregs, approximately 2.6×10⁸ or more Tregs, approximately 2.7×10⁸ or more Tregs, approximately 2.8×10⁸ or more Tregs, approximately 2.9×10⁸ or more Tregs, approximately 3.0×10⁸ or more Tregs, approximately 3.1×10⁸ or more Tregs, approximately 3.2×10⁸ or more Tregs, approximately 3.3×10⁸ or more Tregs, approximately 3.4×10⁸ or more Tregs, approximately 3.5×10⁸ or more Tregs, approximately 3.6×10⁸ or more Tregs, approximately 3.7×10⁸ or more Tregs, approximately 3.8×10⁸ or more Tregs, approximately 3.9×10⁸ or more Tregs, approximately 4.0×10⁸ or more Tregs, approximately 4.1×10⁸ or more Tregs, approximately 4.2×10⁸ or more Tregs, approximately 4.3×10⁸ or more Tregs, approximately 4.4×10⁸ or more Tregs, approximately 4.5×10⁸ or more Tregs, approximately 4.6×10⁸ or more Tregs, approximately 4.7×10⁸ or more Tregs, approximately 4.8×10⁸ or more Tregs, approximately 4.9×10⁸ or more Tregs, approximately 5.0×10⁸ or more Tregs, approximately 5.1×10⁸ or more Tregs, approximately 5.2×10⁸ or more Tregs, approximately 5.3×10⁸ or more Tregs, approximately 5.4×10⁸ or more Tregs, approximately 5.5×10⁸ or more Tregs, approximately 5.6×10⁸ or more Tregs, approximately 5.7×10⁸ or more Tregs, approximately 5.8×10⁸ or more Tregs, approximately 5.9×10⁸ or more Tregs, approximately 6.0×10⁸ or more Tregs, approximately 6.1×10⁸ or more Tregs, approximately 6.2×10⁸ or more Tregs, approximately 6.3×10⁸ or more Tregs, approximately 6.4×10⁸ or more Tregs, approximately 6.5×10⁸ or more Tregs, approximately 6.6×10⁸ or more Tregs, approximately 6.7×10⁸ or more Tregs, approximately 6.8×10⁸ or more Tregs, approximately 6.9×10⁸ or more Tregs, approximately 7.0×10⁸ or more Tregs, approximately 7.1×10⁸ or more Tregs, approximately 7.2×10⁸ or more Tregs, approximately 7.3×10⁸ or more Tregs, approximately 7.4×10⁸ or more Tregs, approximately 7.5×10⁸ or more Tregs, approximately 7.6×10⁸ or more Tregs, approximately 7.7×10⁸ or more Tregs, approximately 7.8×10⁸ or more Tregs, approximately 7.9×10⁸ or more Tregs, approximately 8.0×10⁸ or more Tregs, approximately 8.1×10⁸ or more Tregs, approximately 8.2×10⁸ or more Tregs, approximately 8.3×10⁸ or more Tregs, approximately 8.4×10⁸ or more Tregs, approximately 8.5×10⁸ or more Tregs, approximately 8.6×10⁸ or more Tregs, approximately 8.7×10⁸ or more Tregs, approximately 8.8×10⁸ or more Tregs, approximately 8.9×10⁸ or more Tregs, approximately 9.0×10⁸ or more Tregs, approximately 9.1×10⁸ or more Tregs, approximately 9.2×10⁸ or more Tregs, approximately 9.3×10⁸ or more Tregs, approximately 9.4×10⁸ or more Tregs, approximately 9.5×10⁸ or more Tregs, approximately 9.6×10⁸ or more Tregs, approximately 9.7×10⁸ or more Tregs, approximately 9.8×10⁸ or more Tregs, approximately 9.9×10⁸ or more Tregs, approximately 1.0×10⁹ or more Tregs, approximately 1.1×10⁹ or more Tregs, approximately 1.2×10⁹ or more Tregs, approximately 1.3×10⁹ or more Tregs, approximately 1.4×10⁹ or more Tregs, approximately 1.5×10⁹ or more Tregs, approximately 1.6×10⁹ or more Tregs, approximately 1.7×10⁹ or more Tregs, approximately 1.8×10⁹ or more Tregs, approximately 1.9×10⁹ or more Tregs, approximately 2.0×10⁹ or more Tregs, approximately 2.1×10⁹ or more Tregs, approximately 2.2×10⁹ or more Tregs, approximately 2.3×10⁹ or more Tregs, approximately 2.4×10⁹ or more Tregs, approximately 2.5×10⁹ or more Tregs, approximately 2.6×10⁹ or more Tregs, approximately 2.7×10⁹ or more Tregs, approximately 2.8×10⁹ or more Tregs, approximately 2.9×10⁹ or more Tregs, approximately 3.0×10⁹ or more Tregs, approximately 3.1×10⁹ or more Tregs, approximately 3.2×10⁹ or more Tregs, approximately 3.3×10⁹ or more Tregs, approximately 3.4×10⁹ or more Tregs, approximately 3.5×10⁹ or more Tregs, approximately 3.6×10⁹ or more Tregs, approximately 3.7×10⁹ or more Tregs, approximately 3.8×10⁹ or more Tregs, approximately 3.9×10⁹ or more Tregs, approximately 4.0×10⁹ or more Tregs, approximately 4.1×10⁹ or more Tregs, approximately 4.2×10⁹ or more Tregs, approximately 4.3×10⁹ or more Tregs, approximately 4.4×10⁹ or more Tregs, approximately 4.5×10⁹ or more Tregs, approximately 4.6×10⁹ or more Tregs, approximately 4.7×10⁹ or more Tregs, approximately 4.8×10⁹ or more Tregs, approximately 4.9×10⁹ or more Tregs, or approximately 5.0×10⁹ or more Tregs. In some embodiments, the Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim}FOXP3⁺, CD3⁺FOXP3⁺, CD3⁺CD4⁺FOXP3⁺, CD3⁺CD4⁺CD25 FOXP3⁺, CD3⁺CD25⁺FOXP3⁺, CD3⁺CD25⁺CD127^{dim}, CD4⁺CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺ CD127^{dim}.

A population of cells enriched for Tregs (e.g., the second population of CD45⁺ cells) can have a defined level of purity for Treg cells. For example, a population of cells enriched for Tregs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Treg cells as a percentage of total cells, as a percentage of nucleated cells, or as a percentage of CD45⁺ cells (e.g., where Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim}FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺ CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺ CD127^{dim}).

A population of cells enriched for Tregs (e.g., the second population of CD45⁺ cells) of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95% Tregs as a percentage of total cells, nucleated cells, or CD45⁺ cells (e.g., where the Tregs are CD4⁺ CD25 CD127^{dim}, CD3 CD4⁺ CD25⁺, CD3⁺CD4⁺CD25⁺CD127^{dim} CD3⁺CD4⁺CD25⁺CD127^{dim}FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺ CD127^{dim}).

A population of cells enriched for Tregs of the disclosure (e.g., the second population of CD45⁺ cells) can have a defined level of contaminating non-Treg cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Treg cells per kg of body weight or ideal body weight are present in a population of cells enriched for Tregs of the disclosure, where non-Treg cells are FOXP3⁻ or CD127^{+/bright}.

In some embodiments, a population of cells enriched for Tregs of the disclosure (e.g., the second population of CD45⁺ cells) comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Treg cells, where non-Treg cells are FOXP3⁻ or CD127^{+/bright}.

In various embodiments, the population of cells enriched for Tregs (e.g., the second population of CD45⁺ cells) comprises less than about 5 EU of endotoxins per ml of the solution, less than about 1 EU of endotoxins per ml of the solution, and/or less than about 0.5 EU of endotoxins per ml of the solution.

In some embodiments, at least one of the cell populations comprise less than about 5 EU of endotoxins/ml of respective suspension liquid.

A population of cells enriched for Tregs (e.g., the second population of CD45⁺ cells) can comprise 0.5 EU/ml endotoxins to 10 EU/ml endotoxins. A population of cells enriched for Tregs can comprise at least 0.5 EU/ml endotoxins. A population of cells enriched for Tregs can comprise at most 10 EU/ml endotoxins. A population of cells enriched for Tregs can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A population of cells enriched for Tregs can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of cells enriched for Tregs can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of cells enriched for Tregs can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A population of cells enriched for Tregs (e.g., the second population of CD45⁺ cells) can comprise 0.5% w/w to 10% w/w unbound reagents. These unbound reagents may include any affinity reagents used for the sorting of Tregs, for instance, antibodies, or purification particles or magnetic particles. A population of cells enriched for Tregs can comprise at least 0.5% w/w unbound reagents. A population of cells enriched for Tregs can comprise at most 10% w/w unbound reagents. A population of cells enriched for Tregs can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A population of cells enriched for Tregs can comprise 10% w/w, 8% w/w, 6% w/w, 5% w/w, 4% w/w, 2% w/w, 1% w/w, or 0.5% w/w unbound reagents. A population of cells enriched for Tregs can comprise at least 8% w/w, 6% w/w, 5% w/w, 4% w/w, 2% w/w, 1% w/w, or 0.5% w/w unbound reagents. A population of cells enriched for Tregs can comprise at most 10% w/w, 8% w/w, 6% w/w, 5% w/w, 4% w/w, 2% w/w or 1% w/w unbound reagents.

A population of cells enriched for Tregs (e.g., the second population of CD45⁺ cells) can comprise 1×10³ to 50×10³ microbeads per cell. These microbeads may comprise microbeads used to purify the Treg population, for instance, an anti-CD25 antibody comprising microbead used to sort the Treg population, or an anti-CD4 antibody comprising microbead used to sort the Treg population, and/or an anti-CD127 antibody comprising microbead used to sort the Treg population. A population of cells enriched for Tregs can comprise at least 1×10³ microbeads per cell. A population of cells enriched for Tregs can comprise at most 50×10³ microbeads per cell. A population of cells enriched for Tregs can comprise 50×10³ to 40×10³, 50×10³ to 30×10³, 50×10³ to 20×10³, 50×10³ to 10×10³, 50×10³ to 5×10³, 50×10³ to 4×10³, 50×10³ to 2×10³, 50×10³ to 1×10³, 40×10³ to 30×10³, 40×10³ to 20×10³, 40×10³ to 10×10³, 40×10³ to 5×10³, 40×10³ to 4×10³, 40×10³ to 2×10³, 40×10³ to 1×10³, 30×10³ to 20×10³, 30×10³ to 10×10³, 30×10³ to 5×10³, 30×10³ to 4×10³, 30×10³ to 2×10³, 30×10³ to 1×10³, 20×10³ to 10×10³, 20×10³ to 5×10³, 20×10³ to 4×10³, 20×10³ to 2×10³, 20×10³ to 1×10³, 10×10³ to 5×10³, 10×10³ to 4×10³, 10×10³ to 2×10³, 10×10³ to 1×10³, 5×10³ to 4×10³, 5×10³ to 2×10³, 5×10³ to 1×10³, 4×10³ to 2×10³, 4×10³ to 1×10³, or 2×10³ to 1×10³ microbeads per cell. A population of cells enriched for Tregs can comprise 50×10³, 40×10³, 30×10³, 20×10³, 10×10³, 5×10³, 4×10³, 2×10³, or 1×10³ microbeads per cell.

### Tcons

A third population of CD45⁺ cells that comprises, at least, Tcons or at least one dose of Tcons, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×106, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the third population of CD45⁺ cells and/or Tcons or doses of Tcons per kg of recipient subject's actual body weight or ideal body weight (e.g., where Tcons are CD3⁺ or CD3⁺ CD127⁺/bright).

A third population of CD45⁺ cells that comprises, at least, Tcons can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×107, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ of the third population of CD45⁺ cells and/or Tcons or doses of Tcons per kg of recipient subject's actual body weight or ideal body weight (e.g., where Tcons are CD3⁺ or CD3⁺ CD127^{+/bright})

For example, a third population of CD45⁺ cells that comprises, at least, Tcons or at least one dose of Tcons, can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the third population of CD45⁺ cells and/or Tcons or doses of Tcons per kg of recipient subject's actual body weight or ideal body weight (e.g., where Tcons are CD3⁺ or CD3⁺ CD127⁺/bright).

In some embodiments, the third population of CD45⁺ cells comprising Tcons, or comprising at least one does of Tcons, comprises from approximately 1.0×10⁵ to approximately 1.0×10¹⁰, from approximately 5.0×10⁵ to approximately 6.0×10⁹, from approximately 5.0×10⁵ to approximately 1.0×10⁸, or from approximately 1.5×10⁷ to approximately 6.0×10⁹ Tcons, e.g., in the third population of CD45⁺ cells. In some embodiments, the third population of CD45⁺ cells comprising Tcons, or comprising at least one dose of Tcons, comprises approximately 1.0×10⁵ or more Tcons, approximately 2.0×10⁵ or more Tcons, approximately 3.0×10⁵ or more Tcons, approximately 4.0×10⁵ or more Tcons, approximately 5.0×10⁵ or more Tcons, approximately 6.0×10⁵ or more Tcons, approximately 7.0×10⁵ or more Tcons, approximately 8.0×10⁵ or more Tcons, approximately 9.0×10⁵ or more Tcons, approximately 1.0×10⁶ or more Tcons, approximately 1.0×10⁶ or more Tcons, approximately 1.1×10⁶ or more Tcons, approximately 1.2×10⁶ or more Tcons, approximately 1.3×10⁶ or more Tcons, approximately 1.4×10⁶ or more Tcons, approximately 1.5×10⁶ or more Tcons, approximately 1.6×10⁶ or more Tcons, approximately 1.7×10⁶ or more Tcons, approximately 1.8×10⁶ or more Tcons, approximately 1.9×10⁶ or more Tcons, approximately 2.0×10⁶ or more Tcons, approximately 2.1×10⁶ or more Tcons, approximately 2.2×10⁶ or more Tcons, approximately 2.3×10⁶ or more Tcons, approximately 2.4×10⁶ or more Tcons, approximately 2.5×10⁶ or more Tcons, approximately 2.6×10⁶ or more Tcons, approximately 2.7×10⁶ or more Tcons, approximately 2.8×10⁶ or more Tcons, approximately 2.9×10⁶ or more Tcons, approximately 3.0×10⁶ or more Tcons, approximately 3.1×10⁶ or more Tcons, approximately 3.2×10⁶ or more Tcons, approximately 3.3×10⁶ or more Tcons, approximately 3.4×10⁶ or more Tcons, approximately 3.5×10⁶ or more Tcons, approximately 3.6×10⁶ or more Tcons, approximately 3.7×10⁶ or more Tcons, approximately 3.8×10⁶ or more Tcons, approximately 3.9×10⁶ or more Tcons, approximately 4.0×10⁶ or more Tcons, approximately 4.1×10⁶ or more Tcons, approximately 4.2×10⁶ or more Tcons, approximately 4.3×10⁶ or more Tcons, approximately 4.4×10⁶ or more Tcons, approximately 4.5×10⁶ or more Tcons, approximately 4.6×10⁶ or more Tcons, approximately 4.7×10⁶ or more Tcons, approximately 4.8×10⁶ or more Tcons, approximately 4.9×10⁶ or more Tcons, approximately 5.0×10⁶ or more Tcons, approximately 5.1×10⁶ or more Tcons, approximately 5.2×10⁶ or more Tcons, approximately 5.3×10⁶ or more Tcons, approximately 5.4×10⁶ or more Tcons, approximately 5.5×10⁶ or more Tcons, approximately 5.6×10⁶ or more Tcons, approximately 5.7×10⁶ or more Tcons, approximately 5.8×10⁶ or more Tcons, approximately 5.9×10⁶ or more Tcons, approximately 6.0×10⁶ or more Tcons, approximately 6.1×10⁶ or more Tcons, approximately 6.2×10⁶ or more Tcons, approximately 6.3×10⁶ or more Tcons, approximately 6.4×10⁶ or more Tcons, approximately 6.5×10⁶ or more Tcons, approximately 6.6×10⁶ or more Tcons, approximately 6.7×10⁶ or more Tcons, approximately 6.8×10⁶ or more Tcons, approximately 6.9×10⁶ or more Tcons, approximately 7.0×10⁶ or more Tcons, approximately 7.1×10⁶ or more Tcons, approximately 7.2×10⁶ or more Tcons, approximately 7.3×10⁶ or more Tcons, approximately 7.4×10⁶ or more Tcons, approximately 7.5×10⁶ or more Tcons, approximately 7.6×10⁶ or more Tcons, approximately 7.7×10⁶ or more Tcons, approximately 7.8×10⁶ or more Tcons, approximately 7.9×10⁶ or more Tcons, approximately 8.0×10⁶ or more Tcons, approximately 8.1×10⁶ or more Tcons, approximately 8.2×10⁶ or more Tcons, approximately 8.3×10⁶ or more Tcons, approximately 8.4×10⁶ or more Tcons, approximately 8.5×10⁶ or more Tcons, approximately 8.6×10⁶ or more Tcons, approximately 8.7×10⁶ or more Tcons, approximately 8.8×10⁶ or more Tcons, approximately 8.9×10⁶ or more Tcons, approximately 9.0×10⁶ or more Tcons, approximately 9.1×10⁶ or more Tcons, approximately 9.2×10⁶ or more Tcons, approximately 9.3×10⁶ or more Tcons, approximately 9.4×10⁶ or more Tcons, approximately 9.5×10⁶ or more Tcons, approximately 9.6×10⁶ or more Tcons, approximately 9.7×10⁶ or more Tcons, approximately 9.8×10⁶ or more Tcons, approximately 9.9×10⁶ or more Tcons, approximately 1.0×10⁷ or more Tcons, approximately 1.1×10⁷ or more Tcons, approximately 1.2×10⁷ or more Tcons, approximately 1.3×10⁷ or more Tcons, approximately 1.4×10⁷ or more Tcons, approximately 1.5×10⁷ or more Tcons, approximately 1.6×10⁷ or more Tcons, approximately 1.7×10⁷ or more Tcons, approximately 1.8×10⁷ or more Tcons, approximately 1.9×10⁷ or more Tcons, approximately 2.0×10⁷ or more Tcons, approximately 2.1×10⁷ or more Tcons, approximately 2.2×10⁷ or more Tcons, approximately 2.3×10⁷ or more Tcons, approximately 2.4×10⁷ or more Tcons, approximately 2.5×10⁷ or more Tcons, approximately 2.6×10⁷ or more Tcons, approximately 2.7×10⁷ or more Tcons, approximately 2.8×10⁷ or more Tcons, approximately 2.9×10⁷ or more Tcons, approximately 3.0×10⁷ or more Tcons, approximately 3.1×10⁷ or more Tcons, approximately 3.2×10⁷ or more Tcons, approximately 3.3×10⁷ or more Tcons, approximately 3.4×10⁷ or more Tcons, approximately 3.5×10⁷ or more Tcons, approximately 3.6×10⁷ or more Tcons, approximately 3.7×10⁷ or more Tcons, approximately 3.8×10⁷ or more Tcons, approximately 3.9×10⁷ or more Tcons, approximately 4.0×10⁷ or more Tcons, approximately 4.1×10⁷ or more Tcons, approximately 4.2×10⁷ or more Tcons, approximately 4.3×10⁷ or more Tcons, approximately 4.4×10⁷ or more Tcons, approximately 4.5×10⁷ or more Tcons, approximately 4.6×10⁷ or more Tcons, approximately 4.7×10⁷ or more Tcons, approximately 4.8×10⁷ or more Tcons, approximately 4.9×10⁷ or more Tcons, approximately 5.0×10⁷ or more Tcons, approximately 5.1×10⁷ or more Tcons, approximately 5.2×10⁷ or more Tcons, approximately 5.3×10⁷ or more Tcons, approximately 5.4×10⁷ or more Tcons, approximately 5.5×10⁷ or more Tcons, approximately 5.6×10⁷ or more Tcons, approximately 5.7×10⁷ or more Tcons, approximately 5.8×10⁷ or more Tcons, approximately 5.9×10⁷ or more Tcons, approximately 6.0×10⁷ or more Tcons, approximately 6.1×10⁷ or more Tcons, approximately 6.2×10⁷ or more Tcons, approximately 6.3×10⁷ or more Tcons, approximately 6.4×10⁷ or more Tcons, approximately 6.5×10⁷ or more Tcons, approximately 6.6×10⁷ or more Tcons, approximately 6.7×10⁷ or more Tcons, approximately 6.8×10⁷ or more Tcons, approximately 6.9×10⁷ or more Tcons, approximately 7.0×10⁷ or more Tcons, approximately 7.1×10⁷ or more Tcons, approximately 7.2×10⁷ or more Tcons, approximately 7.3×10⁷ or more Tcons, approximately 7.4×10⁷ or more Tcons, approximately 7.5×10⁷ or more Tcons, approximately 7.6×10⁷ or more Tcons, approximately 7.7×10⁷ or more Tcons, approximately 7.8×10⁷ or more Tcons, approximately 7.9×10⁷ or more Tcons, approximately 8.0×10⁷ or more Tcons, approximately 8.1×10⁷ or more Tcons, approximately 8.2×10⁷ or more Tcons, approximately 8.3×10⁷ or more Tcons, approximately 8.4×10⁷ or more Tcons, approximately 8.5×10⁷ or more Tcons, approximately 8.6×10⁷ or more Tcons, approximately 8.7×10⁷ or more Tcons, approximately 8.8×10⁷ or more Tcons, approximately 8.9×10⁷ or more Tcons, approximately 9.0×10⁷ or more Tcons, approximately 9.1×10⁷ or more Tcons, approximately 9.2×10⁷ or more Tcons, approximately 9.3×10⁷ or more Tcons, approximately 9.4×10⁷ or more Tcons, approximately 9.5×10⁷ or more Tcons, approximately 9.6×10⁷ or more Tcons, approximately 9.7×10⁷ or more Tcons, approximately 9.8×10⁷ or more Tcons, approximately 9.9×10⁷ or more Tcons, approximately 1.0×10⁸ or more Tcons, approximately 1.1×10⁸ or more Tcons, approximately 1.2×10⁸ or more Tcons, approximately 1.3×10⁸ or more Tcons, approximately 1.4×10⁸ or more Tcons, approximately 1.5×10⁸ or more Tcons, approximately 1.6×10⁸ or more Tcons, approximately 1.7×10⁸ or more Tcons, approximately 1.8×10⁸ or more Tcons, approximately 1.9×10⁸ or more Tcons, approximately 2.0×10⁸ or more Tcons, approximately 2.1×10⁸ or more Tcons, approximately 2.2×10⁸ or more Tcons, approximately 2.3×10⁸ or more Tcons, approximately 2.4×10⁸ or more Tcons, approximately 2.5×10⁸ or more Tcons, approximately 2.6×10⁸ or more Tcons, approximately 2.7×10⁸ or more Tcons, approximately 2.8×10⁸ or more Tcons, approximately 2.9×10⁸ or more Tcons, approximately 3.0×10⁸ or more Tcons, approximately 3.1×10⁸ or more Tcons, approximately 3.2×10⁸ or more Tcons, approximately 3.3×10⁸ or more Tcons, approximately 3.4×10⁸ or more Tcons, approximately 3.5×10⁸ or more Tcons, approximately 3.6×10⁸ or more Tcons, approximately 3.7×10⁸ or more Tcons, approximately 3.8×10⁸ or more Tcons, approximately 3.9×10⁸ or more Tcons, approximately 4.0×10⁸ or more Tcons, approximately 4.1×10⁸ or more Tcons, approximately 4.2×10⁸ or more Tcons, approximately 4.3×10⁸ or more Tcons, approximately 4.4×10⁸ or more Tcons, approximately 4.5×10⁸ or more Tcons, approximately 4.6×10⁸ or more Tcons, approximately 4.7×10⁸ or more Tcons, approximately 4.8×10⁸ or more Tcons, approximately 4.9×10⁸ or more Tcons, approximately 5.0×10⁸ or more Tcons, approximately 5.1×10⁸ or more Tcons, approximately 5.2×10⁸ or more Tcons, approximately 5.3×10⁸ or more Tcons, approximately 5.4×10⁸ or more Tcons, approximately 5.5×10⁸ or more Tcons, approximately 5.6×10⁸ or more Tcons, approximately 5.7×10⁸ or more Tcons, approximately 5.8×10⁸ or more Tcons, approximately 5.9×10⁸ or more Tcons, approximately 6.0×10⁸ or more Tcons, approximately 6.1×10⁸ or more Tcons, approximately 6.2×10⁸ or more Tcons, approximately 6.3×10⁸ or more Tcons, approximately 6.4×10⁸ or more Tcons, approximately 6.5×10⁸ or more Tcons, approximately 6.6×10⁸ or more Tcons, approximately 6.7×10⁸ or more Tcons, approximately 6.8×10⁸ or more Tcons, approximately 6.9×10⁸ or more Tcons, approximately 7.0×10⁸ or more Tcons, approximately 7.1×10⁸ or more Tcons, approximately 7.2×10⁸ or more Tcons, approximately 7.3×10⁸ or more Tcons, approximately 7.4×10⁸ or more Tcons, approximately 7.5×10⁸ or more Tcons, approximately 7.6×10⁸ or more Tcons, approximately 7.7×10⁸ or more Tcons, approximately 7.8×10⁸ or more Tcons, approximately 7.9×10⁸ or more Tcons, approximately 8.0×10⁸ or more Tcons, approximately 8.1×10⁸ or more Tcons, approximately 8.2×10⁸ or more Tcons, approximately 8.3×10⁸ or more Tcons, approximately 8.4×10⁸ or more Tcons, approximately 8.5×10⁸ or more Tcons, approximately 8.6×10⁸ or more Tcons, approximately 8.7×10⁸ or more Tcons, approximately 8.8×10⁸ or more Tcons, approximately 8.9×10⁸ or more Tcons, approximately 9.0×10⁸ or more Tcons, approximately 9.1×10⁸ or more Tcons, approximately 9.2×10⁸ or more Tcons, approximately 9.3×10⁸ or more Tcons, approximately 9.4×10⁸ or more Tcons, approximately 9.5×10⁸ or more Tcons, approximately 9.6×10⁸ or more Tcons, approximately 9.7×10⁸ or more Tcons, approximately 9.8×10⁸ or more Tcons, approximately 9.9×10⁸ or more Tcons, approximately 1.0×10⁹ or more Tcons, approximately 1.1×10⁹ or more Tcons, approximately 1.2×10⁹ or more Tcons, approximately 1.3×10⁹ or more Tcons, approximately 1.4×10⁹ or more Tcons, approximately 1.5×10⁹ or more Tcons, approximately 1.6×10⁹ or more Tcons, approximately 1.7×10⁹ or more Tcons, approximately 1.8×10⁹ or more Tcons, approximately 1.9×10⁹ or more Tcons, approximately 2.0×10⁹ or more Tcons, approximately 2.1×10⁹ or more Tcons, approximately 2.2×10⁹ or more Tcons, approximately 2.3×10⁹ or more Tcons, approximately 2.4×10⁹ or more Tcons, approximately 2.5×10⁹ or more Tcons, approximately 2.6×10⁹ or more Tcons, approximately 2.7×10⁹ or more Tcons, approximately 2.8×10⁹ or more Tcons, approximately 2.9×10⁹ or more Tcons, approximately 3.0×10⁹ or more Tcons, approximately 3.1×10⁹ or more Tcons, approximately 3.2×10⁹ or more Tcons, approximately 3.3×10⁹ or more Tcons, approximately 3.4×10⁹ or more Tcons, approximately 3.5×10⁹ or more Tcons, approximately 3.6×10⁹ or more Tcons, approximately 3.7×10⁹ or more Tcons, approximately 3.8×10⁹ or more Tcons, approximately 3.9×10⁹ or more Tcons, approximately 4.0×10⁹ or more Tcons, approximately 4.1×10⁹ or more Tcons, approximately 4.2×10⁹ or more Tcons, approximately 4.3×10⁹ or more Tcons, approximately 4.4×10⁹ or more Tcons, approximately 4.5×10⁹ or more Tcons, approximately 4.6×10⁹ or more Tcons, approximately 4.7×10⁹ or more Tcons, approximately 4.8×10⁹ or more Tcons, approximately 4.9×10⁹ or more Tcons, approximately 5.0×10⁹ or more Tcons, approximately 5.1×10⁹ or more Tcons, approximately 5.2×10⁹ or more Tcons, approximately 5.3×10⁹ or more Tcons, approximately 5.4×10⁹ or more Tcons, approximately 5.5×10⁹ or more Tcons, approximately 5.6×10⁹ or more Tcons, approximately 5.7×10⁹ or more Tcons, approximately 5.8×10⁹ or more Tcons, approximately 5.9×10⁹ or more Tcons, approximately 6.0×10⁹ or more Tcons, approximately 6.1×10⁹ or more Tcons, approximately 6.2×10⁹ or more Tcons, approximately 6.3×10⁹ or more Tcons, approximately 6.4×10⁹ or more Tcons, approximately 6.5×10⁹ or more Tcons, approximately 6.6×10⁹ or more Tcons, approximately 6.7×10⁹ or more Tcons, approximately 6.8×10⁹ or more Tcons, approximately 6.9×10⁹ or more Tcons, approximately 7.0×10⁹ or more Tcons, approximately 7.1×10⁹ or more Tcons, approximately 7.2×10⁹ or more Tcons, approximately 7.3×10⁹ or more Tcons, approximately 7.4×10⁹ or more Tcons, approximately 7.5×10⁹ or more Tcons, approximately 7.6×10⁹ or more Tcons, approximately 7.7×10⁹ or more Tcons, approximately 7.8×10⁹ or more Tcons, approximately 7.9×10⁹ or more Tcons, approximately 8.0×10⁹ or more Tcons, approximately 8.1×10⁹ or more Tcons, approximately 8.2×10⁹ or more Tcons, approximately 8.3×10⁹ or more Tcons, approximately 8.4×10⁹ or more Tcons, approximately 8.5×10⁹ or more Tcons, approximately 8.6×10⁹ or more Tcons, approximately 8.7×10⁹ or more Tcons, approximately 8.8×10⁹ or more Tcons, approximately 8.9×10⁹ or more Tcons, approximately 9.0×10⁹ or more Tcons, approximately 9.1×10⁹ or more Tcons, approximately 9.2×10⁹ or more Tcons, approximately 9.3×10⁹ or more Tcons, approximately 9.4×10⁹ or more Tcons, approximately 9.5×10⁹ or more Tcons, approximately 9.6×10⁹ or more Tcons, approximately 9.7×10⁹ or more Tcons, approximately 9.8×10⁹ or more Tcons, approximately 9.9×10⁹ or more Tcons, or approximately 1.0×10¹⁰ or more Tcons. In some embodiments, the Tcons are CD3⁺ or CD3⁺ CD127^{+/bright}.

A third population of CD45⁺ cells of the disclosure can have a defined level of purity for Tcon cells. For example, a third population of CD45⁺ cells of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tcon cells as a percentage of total cells, as a percentage of nucleated cells, or as a percentage of CD45⁺ cells (e.g., where Tcons are CD3⁺ or CD3⁺ CD127^{+/bright}).

A third population of CD45⁺ cells of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tcons as a percentage of total cells, nucleated cells, or CD45⁺ cells (e.g., where Tcons are CD3⁺ or CD3⁺ CD127^{+/bright})

A third population of CD45⁺ cells of the disclosure can have a defined level of contaminating non-Tcon cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tcon cells per kg of recipient subject's actual body weight or ideal body weight are present in a third population of CD45⁺ cells of the disclosure, (e.g., where non-Tcon cells are CD3⁻ or CD3⁺ CD127^{dim}).

In some embodiments, a third population of CD45⁺ cells of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tcon cells, (e.g., where non-Tcon cells are CD3⁻ or CD3⁺ CD127^{dim}).

In some cases, the population of cells enriched for Tregs comprises less than about 5 EU of endotoxins per ml of the solution, less than about 1 EU of endotoxins per ml of the solution, and/or less than about 0.5 EU of endotoxins per ml of the solution.

In embodiments, at least one of the cell populations comprise less than about 5 EU of endotoxins/ml of respective suspension liquid.

A third population of CD45⁺ cells that comprises, at least, Tcons can comprise 0.5 EU/ml endotoxins to 10 EU/ml endotoxins. A third population of CD45⁺ cells can comprise at least 0.5 EU/ml endotoxins. A third population of CD45⁺ cells can comprise at most 10 EU/ml endotoxins. A third population of CD45⁺ cells can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A third population of CD45⁺ cells can comprise about 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A third population of CD45⁺ cells can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A third population of CD45⁺ cells can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A third population of CD45⁺ cells can comprise less than 0.1% w/w to 3% w/w unbound reagents. These unbound reagents may include any affinity reagents used for the sorting of Tcons or other cell populations, for instance, antibodies, or purification particles or magnetic particles. A third population of CD45⁺ cells can comprise less than about 0.1% w/w unbound reagents. A third population of CD45⁺ cells can comprise less than 3% w/w to 2% w/w, 3% w/w to 1% w/w, 3% w/w to 0.5% w/w, 3% w/w to 0.25% w/w, 3% w/w to 0.1% w/w, 2% w/w to 1% w/w, 2% w/w to 0.5% w/w, 2% w/w to 0.25% w/w, 2% w/w to 0.1% w/w, 1% w/w to 0.5% w/w, 1% w/w to 0.25% w/w, 1% w/w to 0.1% w/w, 0.5% w/w to 0.25% w/w, 0.5% w/w to 0.1% w/w, or 0.25% w/w to 0.1% w/w unbound reagents. A third population of CD45⁺ cells can comprise less than about 3% w/w, 2% w/w, 1% w/w, 0.5% w/w, 0.25% w/w, or 0.1% w/w unbound reagents.

A third population of CD45⁺ cells can comprise less than 50 to 2,000 microbeads per cell. These microbeads may comprise microbeads used to purify the Tcon population or other cell populations, for instance, a CD25 microbead, or a CD4 microbead, or a CD127 microbead, or a CD34 microbead used to sort a cell population. A third population of CD45⁺ cells can comprise less than 2,000 microbeads per cell. A third population of CD45⁺ cells can comprise less than 2,000 to 1,000, 2,000 to 700, 2,000 to 500, 2,000 to 300, 2,000 to 100, 2,000 to 50, 1,000 to 700, 1,000 to 500, 1,000 to 300, 1,000 to 100, 1,000 to 50, 700 to 500, 700 to 300, 700 to 100, 700 to 50, 500 to 300, 500 to 100, 500 to 50, 300 to 100, 300 to 50, or 100 to 50 microbeads per cell. A third population of CD45⁺ cells can comprise about 2,000, 1,000, 700, 500, 300, 100, or 50 microbeads per cell. A third population of CD45⁺ cells can comprise no microbeads per cell.

In a third population of CD45⁺ cells, the ratio of Tcons:Tregs administered to a subject can be, for example, about 1:100, 1:50, 1:25, 1:20, 1:15, 1:10, 1:9, 1:8, 1:7, 1:6, 1:5, 1:4, 1:3, 1:2.5, 1:2, 1.5:2, 1:1.5, 1:1, 1.5:1, 2:1, 2:1.5, 2.5:1, 3:1, 4:1, 5:1, 6:1, 7:1, 8:1, 9:1, 10:1, 15:1, 20:1, 25:1, 50:1, or 100:1.

Cells of the third population of CD45⁺ cells that comprises, at least, Tcons can be cryopreserved for any amount of time. Cells of the third population of CD45⁺ cells may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, cells of the third population of CD45⁺ cells that comprises, at least, Tcons are cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, cells of the third population of CD45⁺ cells are cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, cells of the third population of CD45⁺ cells are cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

iNKTs

A cell population that comprises a population of iNKTs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more iNKTs per kg of recipient subject's actual body weight or ideal body weight (e.g., where iNKTs are CD3⁺Vα24Jα18⁺).

A cell population that comprises a population of iNKTs can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×107, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×107, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ iNKTs per kg of recipient subject's actual body weight or ideal body weight (e.g., where iNKTs are CD3⁺Vα24Jα18⁺).

For example, a cell population that comprises a population of iNKTs can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷ 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ iNKTs per kg of recipient subject's actual body weight or ideal body weight (e.g., where iNKTs are CD3⁺Vα24Jα18⁺).

A population of iNKTs of the disclosure can have a defined level of purity for iNKT cells. For example, a population of iNKTs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more iNKT cells as a percentage of total cells, as a percentage of nucleated cells, or as a percentage of CD45⁺ cells (e.g., where iNKTs are CD3⁺Vα24Jα18⁺).

A population of iNKTs of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, iNKTs as a percentage of total cells, nucleated cells, or CD45⁺ cells (e.g., where iNKTs are CD3⁺Vα24Jα18⁺).

A population of iNKTs of the disclosure can have a defined level of contaminating non-iNKT cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-iNKT cells per kg of recipient subject's actual body weight or ideal body weight are present in a population of iNKTs of the disclosure, (e.g., where non-iNKT cells are Vα24Jα18⁻).

In some embodiments, a population of iNKTs of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-iNKT cells, (e.g., where non-iNKT cells are Vα24Jα18″).

### Tmems

A cell population that comprises a population of Tmems can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tmems per kg of recipient subject's actual body weight or ideal body weight (e.g., where Tmems are CD3⁺CD45RA⁻CD45RO⁺).

A cell population that comprises a population of Tmems can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ Tmems per kg of recipient subject's actual body weight or ideal body weight (e.g., where Tmems are CD3⁺ CD45RA⁻CD45RO⁺).

For example, a cell population that comprises a population of Tmems can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tmems per kg of recipient subject's actual body weight or ideal body weight (e.g., where Tmems are CD3⁺ CD45RA⁻CD45RO⁺).

A population of Tmems of the disclosure can have a defined level of purity for Tmem cells. For example, a population of Tmems of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tmem cells as a percentage of total cells, as a percentage of nucleated cells, or as a percentage of CD45⁺ cells (e.g., where Tmems are CD3⁺CD45RA⁻CD45RO⁺).

A population of Tmems of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tmems as a percentage of total cells, nucleated cells, or CD45⁺ cells (e.g., where Tmems are CD3⁺ CD45RA⁻CD45RO⁺).

A population of Tmems of the disclosure can have a defined level of contaminating non-Tmem cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tmem cells per kg of recipient subject's actual body weight or ideal body weight are present in a population of Tmems of the disclosure, (e.g., where the non-Tmem cells are CD45RO⁻).

In some embodiments, a population of Tmems of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tmem cells, (e.g., where the non-Tmem cells are CD45RO⁻).

### Sequence and Timing of Administration of Cell Populations

An aspect provides a multi-component pharmaceutical treatment or multi-component cellular therapy product to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs), e.g., a second population of CD45⁺ cells; (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent. In some embodiments, the multi-component cellular therapy product comprises (a) a first single dose transfer bag comprising a first population of isolated CD45⁺ cells formulated with an excipient at a neutral pH, and comprising at least one dose of CD34⁺ hematopoietic stem and progenitor cells (HSPCs); (b) a second single dose transfer bag comprising a second population of isolated CD45⁺ cells formulated with an excipient at a neutral pH, and comprising at least one dose of fresh CD4⁺CD25⁺CD127^{dim} regulatory T cells (Tregs); and (c) a third single dose transfer bag comprising a third population of isolated CD45⁺ cells formulated with an excipient at a neutral pH, and comprising at least one dose of conventional CD3⁺ T cells (Tcons).

Another aspect provides a method of treating a human subject having or suspected of having a hematologic malignancy. In some embodiments, the human subject is or has been diagnosed with the hematologic malignancy. In some embodiments, the method comprises administering to the human subject a solution comprising the first population of CD45⁺ cells, a solution comprising the population of cells enriched for regulatory T cells (Tregs), e.g., a second population of CD45⁺ cells, a solution comprising the third population of CD45⁺ cells, and a solution comprising one or more doses of the GVHD prophylactic agent. In this embodiment, the solution comprising the first population of CD45⁺ cells, the solution comprising the population of cells enriched for regulatory Tregs, the solution comprising the third population of CD45⁺ cells, and the solution comprising one or more doses of the GVHD prophylactic agent are as defined according to any herein disclosed multi-component pharmaceutical treatment or multi-component cellular therapy product. In some embodiments, the method comprises administering to the human subject a solution comprising a first population of isolated CD45⁺ cells comprising CD34⁺ hematopoietic stem and progenitor cells (HSPCs), a solution comprising a second population of isolated CD45⁺ cells comprising fresh CD4⁺CD25⁺CD127^{dim} regulatory T cells (Tregs), and a solution comprising a third population of isolated CD45⁺ cells comprising conventional CD3⁺ T cells (Tcons). This embodiment may further comprise administering a solution comprising one or more doses of a graft vs host disease (GVHD) prophylactic agent. In this embodiment, the solution comprising the first population of CD45⁺ cells, the solution comprising the second population of CD45⁺ cells, the solution comprising the third population of CD45⁺ cells, and the solution comprising one or more doses of the GVHD prophylactic agent are as defined according to any herein disclosed multi-component pharmaceutical treatment or multi-component cellular therapy product.

Disclosed herein are methods for enhanced allogeneic hematopoietic stem cell transplantation, comprising administering to a subject solutions that comprise populations of cells.

In some embodiments, cell populations that comprise a first population of CD45⁺ cells which comprises at least HSPCs, a population of cells enriched for regulatory T cells (Tregs), and a third population of CD45⁺ cells which comprises at least Tcons are administered to a subject.

The first population of CD45⁺ cells and the population of cells enriched for Tregs can be administered at the same or similar times, or at different times. In some embodiments, first population of CD45⁺ cells and the population of cells enriched for Tregs are administered on the same day.

In various embodiments, the first population of CD45⁺ cells and the population of cells enriched for Tregs are administered before the third population of CD45⁺ cells.

The first population of CD45⁺ cells and the population of cells enriched for Tregs can administered at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours apart.

The third population of CD45⁺ cells can be administered to the subject after the first population of CD45⁺ cells.

The third population of CD45⁺ cells can be administered to the subject at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

In some embodiments, the third population of CD45⁺ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 120 hours after the first population of CD45⁺ cells.

The third population of CD45⁺ cells can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells.

The third population of CD45⁺ cells can be administered to the subject after the population of cells enriched for Tregs.

The population Tcons can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the third population of CD45⁺ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The third population of CD45⁺ cells can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

The third population of CD45⁺ cells can be administered to the subject after the first population of CD45⁺ cells and the population of cells enriched for Tregs.

The third population of CD45⁺ cells can be administered to the subject, for example, greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells and the population of cells enriched for Tregs.

In some embodiments, the third population of CD45⁺ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells and the population of cells enriched for Tregs.

The third population of CD45⁺ cells can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells and the population of cells enriched for Tregs.

In some embodiments, a population of hematopoietic stem and progenitor cells (HSPCs), a population of cells enriched for regulatory T cells (Tregs), a population of conventional T cells (Tcons), and a population of invariant natural killer T cells (iNKTs) are administered to a subject.

The population of iNKTs can be administered to the subject at the same time or at a similar time as the first population of CD45⁺ cells. In some embodiments, the population of iNKTs is administered to the subject after the first population of CD45⁺ cells.

The population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

In some embodiments, the population of iNKTs is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

The population of iNKTs can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells.

The population of iNKTs can be administered to the subject at the same time or at a similar time as the population of cells enriched for Tregs. In some embodiments, the population of iNKTs is administered to the subject after the population of cells enriched for Tregs.

A population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the population of iNKTs is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The population of iNKTs can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

In some embodiments, a population of hematopoietic stem and progenitor cells (HSPCs), a population of cells enriched for regulatory T cells (Tregs), a population of conventional T cells (Tcons), and a population of memory T cells (Tmems) are administered to a subject.

A population of Tmems can be administered to the subject at the same time or at a similar time as the first population of CD45⁺ cells. In some embodiments, the population of Tmems is administered to the subject after the first population of CD45⁺ cells.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

The population of Tmems can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells.

The population of Tmems can be administered to the subject at the same time or at a similar time as the population of cells enriched for Tregs. In some embodiments, the population of Tmems is administered to the subject after the population of cells enriched for Tregs.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The population of Tmems can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

In some embodiments, the population of Tcons is administered at least about 12 hours after the population of HSPCs, e.g., the population of Tcons is administered from approximately 24 to approximately 96 hours after the population of HSPCs or the population of Tcons is administered from approximately 36 to approximately 60 hours after the population of HSPCs.

In embodiments, the population of Tcons is administered at least about 12 hours after the population of cells comprising Tregs, e.g., the population of Tcons is administered from approximately 24 to approximately 96 hours after the population of cells comprising Tregs or the population of Tcons is administered from approximately 36 to approximately 60 hours after the population of cells comprising Tregs.

### Heterogenous Cell Populations

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes, and a liquid suspending the cells. In this aspect, at least about 30% of the lymphocytes comprise Tcons. and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending the cells. In this aspect, at least about 30% of the lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

In various embodiments, the heterogenous cell population comprises from approximately 0.2 to approximately 2.0 percent hematopoietic stem and progenitor cells.

In some embodiments, the hematologic malignancy is leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN), or any combinations thereof.

In embodiments, a genetic expression level of the T-reg cells correlates to cells that were harvested from the donor within about 60 hours prior to administration to the patient.

In various embodiments, the number of T-reg cells in the T-reg population is about equal to the number of T-con cells in the heterogenous cell population. In some cases, the T-reg cells in the T-reg population inhibit activation of conventional T cells in the heterogenous cell population by the patient's healthy tissue by an amount up to approximately 20 percent.

In some embodiments, the peripheral blood of the patient exhibits an elevated ratio of Tregs to CD4⁺ T cells up to approximately 100 days after administration of the cell populations as compared to a healthy human subject that was not administered the cells populations.

In embodiments, at least about 50% of the cells in the HSPC's cell population are colony forming units.

In various embodiments, at least one of the cell populations has an elevated amount of granulocyte colony-stimulating factor as compared to non-mobilized blood. In some cases, the at least one cell populations is the heterogenous cell population.

An aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In some embodiments, a heterogenous cell population may be administered to a subject. A heterogenous cell population may comprise many different cell types found in the peripheral blood of a human donor. A heterogenous cell population may comprise granulocytes, monocytes, and lymphocytes. A heterogenous cell population may comprise T cells (such as Tcons, Tregs, Tmems, naïve T cells, CD4⁺ T cells, NK-T cells), B cells, NK cells, HSPCs, dendritic cells (such as plasmacytoid dendritic cells and myeloid dendritic cells) and other cell populations found in peripheral blood. A heterogenous cell population may be administered to a subject in addition to the other populations described herein. For instance, a heterogenous cell population may be administered with HSPCs as described herein. In some cases, a heterogenous cell population may be administered with HSPCs and Tregs as described herein. In some cases, a heterogenous cell population may be administered with Tregs as described herein. In some cases, a heterogenous cell population may be administered with Tcons as described herein. In some cases, a heterogenous cell population may be administered instead of the Tcon population as described herein.

In some embodiments, a heterogenous cell population administered to a subject may comprise a combination of granulocytes and monocytes. A combination of granulocytes and monocytes may comprise from 30% to 80% of the heterogenous cell population. At least 30% of the heterogenous cell population may comprise a combination of granulocytes and monocytes. At most 80% of the heterogenous cell population may comprise a combination of granulocytes and monocytes. In some cases, 30% to 40%, 30% to 50%, 30% to 60%, 30% to 70%, 30% to 80%, 40% to 50%, 40% to 60%, 40% to 70%, 40% to 80%, 50% to 60%, 50% to 70%, 50% to 80%, 60% to 70%, 60% to 80%, or 70% to 80% of the heterogenous cell population may comprise a combination of granulocytes and monocytes. In some cases, 30%, 40%, 50%, 60%, 70%, or 80% of the heterogenous cell population may comprise a combination of granulocytes and monocytes. In some cases, at least 30%, 40%, 50%, 60% or 70% of the heterogenous cell population may comprise a combination of granulocytes and monocytes. In some cases, at most 40%, 50%, 60%, 70%, or 80% of the heterogenous cell population may comprise a combination of granulocytes and monocytes.

In some embodiments, a heterogenous cell population administered to the subject may comprise lymphocytes. Lymphocytes comprise CD45⁺ cells. Lymphocytes may comprise from 8% to 50% of the heterogenous cell population. In some cases, at least 8% of the heterogenous cell population may comprise lymphocytes. In some cases, at most 50% of the heterogenous cell population may comprise lymphocytes. In some cases, 8% to 10%, 8% to 20%, 8% to 25%, 8% to 30%, 8% to 40%, 8% to 45%, 8% to 50%, 10% to 20%, 10% to 25%, 10% to 30%, 10% to 40%, 10% to 45%, 10% to 50%, 20% to 25%, 20% to 30%, 20% to 40%, 20% to 45%, 20% to 50%, 25% to 30%, 25% to 40%, 25% to 45%, 25% to 50%, 30% to 40%, 30% to 45%, 30% to 50%, 40% to 45%, 40% to 50%, or 45% to 50% of the heterogenous cell population may comprise lymphocytes. In some cases, 8%, 10%, 20%, 25%, 30%, 40%, 45%, or 50% of the heterogenous cell population may comprise lymphocytes. In some cases, at least 8%, 10%, 20%, 25%, 30%, 40% or 45% of the heterogenous cell population may comprise lymphocytes. In some cases, at most 10%, 20%, 25%, 30%, 40%, 45%, or 50% of the heterogenous cell population may comprise lymphocytes.

In some embodiments, lymphocytes in the heterogenous cell population may comprise Tcons. Tcons may comprise from 40% to 85% of the lymphocyte subset of the heterogenous cell population. In some cases, at least 40% of the lymphocyte subset may comprise Tcons. In some cases, at most 85% of the lymphocyte subset may comprise Tcons. In some cases, 40% to 50%, 40% to 60%, 40% to 65%, 40% to 70%, 40% to 75%, 40% to 80%, 40% to 85%, 50% to 60%, 50% to 65%, 50% to 70%, 50% to 75%, 50% to 80%, 50% to 85%, 60% to 65%, 60% to 70%, 60% to 75%, 60% to 80%, 60% to 85%, 65% to 70%, 65% to 75%, 65% to 80%, 65% to 85%, 70% to 75%, 70% to 80%, 70% to 85%, 75% to 80%, 75% to 85%, or 80% to 85% of the lymphocyte subset may comprise Tcons. In some cases, 40%, 50%, 60%, 65%, 70%, 75%, 80%, or 85% of the lymphocyte subset may comprise Tcons. In some cases, at least 40%, 50%, 60%, 65%, 70%, 75% or 80% of the lymphocyte subset may comprise Tcons. In some cases, at most 50%, 60%, 65%, 70%, 75%, 80%, or 85% of the lymphocyte subset may comprise Tcons.

In some embodiments, CD3⁺ lymphocytes in the heterogenous cell population may comprise CD4⁺ T cells. In some cases, 30% to 70% of the CD3⁺ lymphocyte subset may comprise CD4⁺ T cells. In some cases, at least 30% of the CD3⁺ lymphocyte subset may comprise CD4⁺ T cells. In some cases, at most 70% of the CD3⁺ lymphocyte subset may comprise CD4⁺ T cells. In some cases, 30% to 40%, 30% to 50%, 30% to 60%, 30% to 70%, 40% to 50%, 40% to 60%, 40% to 70%, 50% to 60%, 50% to 70%, or 60% to 70% of the CD3⁺ lymphocyte subset may comprise CD4⁺ T cells. In some cases, 30%, 40%, 50%, 60%, or 70% of the CD3⁺ lymphocyte subset may comprise CD4⁺ T cells. In some cases, at least 30%, 40%, 50% or 60% of the CD3⁺ lymphocyte subset may comprise CD4⁺ T cells. In some cases, at most 40%, 50%, 60%, or 70% of the CD3⁺ lymphocyte subset may comprise CD4⁺ T cells.

In some embodiments, CD3⁺ lymphocytes in the heterogenous cell population may comprise CD8⁺ T cells. In some cases, 20% to 65% of the CD3⁺ lymphocyte subset may comprise CD8⁺ T cells. In some cases, at least 20% of the CD3⁺ lymphocyte subset may comprise CD8⁺ T cells. In some cases, at most 65% of the CD3⁺ lymphocyte subset may comprise CD8⁺ T cells. In some cases, 20% to 30%, 20% to 40%, 20% to 50%, 20% to 60%, 20% to 65%, 30% to 40%, 30% to 50%, 30% to 60%, 30% to 65%, 40% to 50%, 40% to 60%, 40% to 65%, 50% to 60%, 50% to 65%, or 60% to 65% of the CD3⁺ lymphocyte subset may comprise CD8⁺ T cells. In some cases, 20%, 30%, 40%, 50%, 60%, or 65% of the CD3⁺ lymphocyte subset may comprise CD8⁺ T cells. In some cases, at least 20%, 30%, 40%, 50% or 60% of the CD3⁺ lymphocyte subset may comprise CD8⁺ T cells. In some cases, at most 30%, 40%, 50%, 60%, or 65% of the CD3⁺ lymphocyte subset may comprise CD8⁺ T cells.

In some embodiments, lymphocytes in the heterogenous cell population may comprise B cells. In some cases, 4% to 35% of the lymphocyte subset may comprise B cells. In some cases, at least 4% of the lymphocyte subset may comprise B cells. In some cases, at most 35% of the lymphocyte subset may comprise B cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise B cells. In some cases, 4%, 5%, 10%, 20%, 30%, or 35% of the lymphocyte subset may comprise B cells. In some cases, at least 4%, 5%, 10%, 20% or 30% of the lymphocyte subset may comprise B cells. In some cases, at most 5%, 10%, 20%, 30%, or 35% of the lymphocyte subset may comprise B cells. B cells may be CD45⁺ CD19⁺ or CD45⁺CD19⁺CD3− cells.

In some embodiments, lymphocytes in the heterogenous cell population may comprise NK cells. In some cases, 4% to 35% of the lymphocyte subset may comprise NK cells. In some cases, at least 4% of the lymphocyte subset may comprise NK cells. In some cases, at most 35% of the lymphocyte subset may comprise NK cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise NK cells. In some cases, 4%, 5%, 10%, 20%, 30%, or 35% of the lymphocyte subset may comprise NK cells. In some cases, at least 4%, 5%, 10%, 20% or 30% of the lymphocyte subset may comprise NK cells. In some cases, at most 5%, 10%, 20%, 30%, or 35% of the lymphocyte subset may comprise NK cells. NK cells may be CD45⁺ CD56⁺ or CD45⁺CD56⁺CD3− cells.

In some embodiments, CD3⁺ lymphocytes in the heterogenous cell population may comprise NK-T cells. In some cases, 3% to 30% of the CD3⁺ lymphocyte subset may comprise NK-T cells. In some cases, at least 4% of the CD3⁺ lymphocyte subset may comprise NK-T cells. In some cases, at most 35% of the CD3⁺ lymphocyte subset may comprise NK-T cells. In some cases, 3% to 5%, 3% to 10%, 3% to 20%, 3% to 30%, 5% to 10%, 5% to 20%, 5% to 30%, 10% to 20%, 10% to 30%, 20% to 30%, of the CD3⁺ lymphocyte subset may comprise NK-T cells. In some cases, 3%, 5%, 10%, 20% or 30% of the CD3⁺ lymphocyte subset may comprise NK-T cells. In some cases, at least 3%, 5%, 10% or 20% of the CD3⁺ lymphocyte subset may comprise NK-T cells. In some cases, at most 10%, 20% or 30% of the CD3⁺ lymphocyte subset may comprise NK-T cells. NK-T cells may be CD45⁺ CD56⁺ or CD45⁺CD56⁺CD3⁺ cells.

In some embodiments, lymphocytes in the heterogenous cell population may comprise CD34⁺ cells. In some cases, 0.1% to 2% of the lymphocyte subset may comprise CD34⁺ cells. In some cases, at least 0.1% of the lymphocyte subset may comprise CD34⁺ cells. In some cases, at most 2% of the lymphocyte subset may comprise CD34⁺ cells. In some cases, 0.1% to 0.5%, 0.1% to 1%, 0.1% to 1.5%, 0.1% to 2%, 0.5% to 1%, 0.5% to 1.5%, 0.5% to 2%, 1% to 1.5%, 1% to 2%, or 1.5% to 2% of the lymphocyte subset may comprise CD34⁺ cells. In some cases, 0.1%, 0.5%, 1%, 1.5%, or 2% of the lymphocyte subset may comprise CD34⁺ cells. In some cases, at least 0.1%, 0.5%, 1% or 1.5% of the lymphocyte subset may comprise CD34⁺ cells. In some cases, at most 0.5%, 1%, 1.5%, or 2% of the lymphocyte subset may comprise CD34⁺ cells.

### GVHD Prophylactic Agents

Subjects administered a composition of the disclosure (e.g., a cell component comprising a populations of cells described herein) and a GVHD prophylactic agent (for example, a single GVHD prophylactic agent) exhibit a low incidence of ≥grade 1 aGVHD, for example, a lower incidence of ≥grade 1 aGVHD than subjects that are administered an alternate composition. A single GVHD prophylactic agent can be a calcineurin inhibitor such as tacrolimus or another agent which acts on the same targets as tacrolimus or comprises an active fragment of tacrolimus. In some embodiments, a low dose of a GVHD prophylactic agent is tacrolimus with a target trough level (e.g., trough blood level) of approximately 5 ng/ml to approximately 10 ng/ml. In some embodiments, a low dose of a GVHD prophylactic agent is tacrolimus with a target trough level of approximately 4 ng/ml to approximately 6 ng/mL. In some embodiments, a low dose of a GVHD prophylactic agent is tacrolimus with a target trough level (e.g., target trough blood level) of approximately 1 ng/ml or more, approximately 2 ng/ml or more, approximately 3 ng/ml or more, approximately 4 ng/ml or more, approximately 5 ng/ml or more, approximately 6 ng/ml or more, approximately 7 ng/mL or more, approximately 8 ng/mL or more, approximately 9 ng/ml or more, approximately 10 ng/ml or more, approximately 11 ng/mL or more, approximately 12 ng/ml or more, approximately 13 ng/ml or more, approximately 14 ng/ml or more, approximately 15 ng/ml or more, approximately 16 ng/ml or more, approximately 17 ng/ml or more, approximately 18 ng/ml or more, approximately 19 ng/ml or more, or approximately 20 ng/mL or more.

In some embodiments, the single GVHD prophylactic agent is tacrolimus, and the tacrolimus is provided at a dose that ranges from approximately 0.01 mg per kilogram of body weight of the subject to approximately 0.50 mg per kilogram of body weight of the subject twice per day. In some embodiments, the tacrolimus is provided at a dose of approximately 0.01 mg per kilogram of body weight of the subject twice per day, approximately 0.02 mg per kilogram of body weight of the subject twice per day, approximately 0.03 mg per kilogram of body weight of the subject twice per day, approximately 0.04 mg per kilogram of body weight of the subject twice per day, approximately 0.05 mg per kilogram of body weight of the subject twice per day, approximately 0.06 mg per kilogram of body weight of the subject twice per day, approximately 0.07 mg per kilogram of body weight of the subject twice per day, approximately 0.08 mg per kilogram of body weight of the subject twice per day, approximately 0.09 mg per kilogram of body weight of the subject twice per day, approximately 0.10 mg per kilogram of body weight of the subject twice per day, approximately 0.11 mg per kilogram of body weight of the subject twice per day, approximately 0.12 mg per kilogram of body weight of the subject twice per day, approximately 0.13 mg per kilogram of body weight of the subject twice per day, approximately 0.14 mg per kilogram of body weight of the subject twice per day, approximately 0.15 mg per kilogram of body weight of the subject twice per day, approximately 0.16 mg per kilogram of body weight of the subject twice per day, approximately 0.17 mg per kilogram of body weight of the subject twice per day, approximately 0.18 mg per kilogram of body weight of the subject twice per day, approximately 0.19 mg per kilogram of body weight of the subject twice per day, approximately 0.20 mg per kilogram of body weight of the subject twice per day, approximately 0.21 mg per kilogram of body weight of the subject twice per day, approximately 0.22 mg per kilogram of body weight of the subject twice per day, approximately 0.23 mg per kilogram of body weight of the subject twice per day, approximately 0.24 mg per kilogram of body weight of the subject twice per day, approximately 0.25 mg per kilogram of body weight of the subject twice per day, approximately 0.26 mg per kilogram of body weight of the subject twice per day, approximately 0.27 mg per kilogram of body weight of the subject twice per day, approximately 0.28 mg per kilogram of body weight of the subject twice per day, approximately 0.29 mg per kilogram of body weight of the subject twice per day, approximately 0.30 mg per kilogram of body weight of the subject twice per day, approximately 0.31 mg per kilogram of body weight of the subject twice per day, approximately 0.32 mg per kilogram of body weight of the subject twice per day, approximately 0.33 mg per kilogram of body weight of the subject twice per day, approximately 0.34 mg per kilogram of body weight of the subject twice per day, approximately 0.35 mg per kilogram of body weight of the subject twice per day, approximately 0.36 mg per kilogram of body weight of the subject twice per day, approximately 0.37 mg per kilogram of body weight of the subject twice per day, approximately 0.38 mg per kilogram of body weight of the subject twice per day, approximately 0.39 mg per kilogram of body weight of the subject twice per day, approximately 0.40 mg per kilogram of body weight of the subject twice per day, approximately 0.41 mg per kilogram of body weight of the subject twice per day, approximately 0.42 mg per kilogram of body weight of the subject twice per day, approximately 0.43 mg per kilogram of body weight of the subject twice per day, approximately 0.44 mg per kilogram of body weight of the subject twice per day, approximately 0.45 mg per kilogram of body weight of the subject twice per day, approximately 0.46 mg per kilogram of body weight of the subject twice per day, approximately 0.47 mg per kilogram of body weight of the subject twice per day, approximately 0.48 mg per kilogram of body weight of the subject twice per day, approximately 0.49 mg per kilogram of body weight of the subject twice per day, or approximately 0.50 mg per kilogram of body weight of the subject twice per day.

A single GVHD prophylactic agent can be sirolimus. In some embodiments, a low dose of a GVHD prophylactic agent is sirolimus with a target trough level (e.g., trough blood level) of approximately 3 ng/ml to approximately 8 ng/mL. In some embodiments, a low dose of a GVHD prophylactic agent is sirolimus with a target trough level of approximately 4 ng/ml to approximately 8 ng/mL. In some embodiments, a low dose of a GVHD prophylactic agent is sirolimus with a target trough level (e.g., target trough blood level) of approximately 1 ng/ml or more, approximately 2 ng/ml or more, approximately 3 ng/mL or more, approximately 4 ng/ml or more, approximately 5 ng/ml or more, approximately 6 ng/ml or more, approximately 7 ng/ml or more, approximately 8 ng/ml or more, approximately 9 ng/ml or more, approximately 10 ng/mL or more, approximately 11 ng/mL or more, approximately 12 ng/ml or more, approximately 13 ng/mL or more, approximately 14 ng/ml or more, approximately 15 ng/ml or more, approximately 16 ng/mL or more, approximately 17 ng/ml or more, approximately 18 ng/ml or more, approximately 19 ng/ml or more, or approximately 20 ng/ml or more.

In some embodiments, the single GVHD prophylactic agent is sirolimus, and the sirolimus is provided at a dose that ranges from approximately 0.01 mg per kilogram of body weight of the subject to approximately 0.50 mg per kilogram of body weight of the subject twice per day. In some embodiments, the sirolimus is provided at a dose of approximately 0.01 mg per kilogram of body weight of the subject twice per day, approximately 0.02 mg per kilogram of body weight of the subject twice per day, approximately 0.03 mg per kilogram of body weight of the subject twice per day, approximately 0.04 mg per kilogram of body weight of the subject twice per day, approximately 0.05 mg per kilogram of body weight of the subject twice per day, approximately 0.06 mg per kilogram of body weight of the subject twice per day, approximately 0.07 mg per kilogram of body weight of the subject twice per day, approximately 0.08 mg per kilogram of body weight of the subject twice per day, approximately 0.09 mg per kilogram of body weight of the subject twice per day, approximately 0.10 mg per kilogram of body weight of the subject twice per day, approximately 0.11 mg per kilogram of body weight of the subject twice per day, approximately 0.12 mg per kilogram of body weight of the subject twice per day, approximately 0.13 mg per kilogram of body weight of the subject twice per day, approximately 0.14 mg per kilogram of body weight of the subject twice per day, approximately 0.15 mg per kilogram of body weight of the subject twice per day, approximately 0.16 mg per kilogram of body weight of the subject twice per day, approximately 0.17 mg per kilogram of body weight of the subject twice per day, approximately 0.18 mg per kilogram of body weight of the subject twice per day, approximately 0.19 mg per kilogram of body weight of the subject twice per day, approximately 0.20 mg per kilogram of body weight of the subject twice per day, approximately 0.21 mg per kilogram of body weight of the subject twice per day, approximately 0.22 mg per kilogram of body weight of the subject twice per day, approximately 0.23 mg per kilogram of body weight of the subject twice per day, approximately 0.24 mg per kilogram of body weight of the subject twice per day, approximately 0.25 mg per kilogram of body weight of the subject twice per day, approximately 0.26 mg per kilogram of body weight of the subject twice per day, approximately 0.27 mg per kilogram of body weight of the subject twice per day, approximately 0.28 mg per kilogram of body weight of the subject twice per day, approximately 0.29 mg per kilogram of body weight of the subject twice per day, approximately 0.30 mg per kilogram of body weight of the subject twice per day, approximately 0.31 mg per kilogram of body weight of the subject twice per day, approximately 0.32 mg per kilogram of body weight of the subject twice per day, approximately 0.33 mg per kilogram of body weight of the subject twice per day, approximately 0.34 mg per kilogram of body weight of the subject twice per day, approximately 0.35 mg per kilogram of body weight of the subject twice per day, approximately 0.36 mg per kilogram of body weight of the subject twice per day, approximately 0.37 mg per kilogram of body weight of the subject twice per day, approximately 0.38 mg per kilogram of body weight of the subject twice per day, approximately 0.39 mg per kilogram of body weight of the subject twice per day, approximately 0.40 mg per kilogram of body weight of the subject twice per day, approximately 0.41 mg per kilogram of body weight of the subject twice per day, approximately 0.42 mg per kilogram of body weight of the subject twice per day, approximately 0.43 mg per kilogram of body weight of the subject twice per day, approximately 0.44 mg per kilogram of body weight of the subject twice per day, approximately 0.45 mg per kilogram of body weight of the subject twice per day, approximately 0.46 mg per kilogram of body weight of the subject twice per day, approximately 0.47 mg per kilogram of body weight of the subject twice per day, approximately 0.48 mg per kilogram of body weight of the subject twice per day, approximately 0.49 mg per kilogram of body weight of the subject twice per day, or approximately 0.50 mg per kilogram of body weight of the subject twice per day.′

Methods for alloHSCT of the disclosure utilize tacrolimus. Combining tacrolimus with one or more cell populations in an alloHSCT regimen as disclosed herein is shown to result in surprising improvements in clinical outcomes.

An aspect provides a multi-component pharmaceutical treatment or multi-component cellular therapy product to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent, wherein the GVHD prophylactic agent is tacrolimus. In some embodiments, the multi-component cellular therapy product comprises a) a first single dose transfer bag comprising a first population of isolated CD45⁺ cells comprising a dose of CD34⁺ hematopoietic stem and progenitor cells (HSPCs) formulated with an excipient at a neutral pH; b) a second single dose transfer bag comprising a second population of isolated CD45⁺ cells comprising a dose of fresh CD4⁺ CD25 CD127^{dim} regulatory T cells (Tregs) formulated with an excipient at a neutral pH; and c) a third single dose transfer bag comprising a third population of isolated CD45⁺ cells comprising a dose of conventional CD3⁺ T cells (Tcons) formulated with an excipient at a neutral pH, wherein the excipient comprises one more cryoprotectants.

In various embodiments, the tacrolimus is administered or dosed in an amount to maintain or that maintains a target blood level of at least about 3 ng/ml for at least about 20 days after administering the third population of CD45⁺ cells, in an amount to maintain a target blood level of approximately 4 ng/ml or more for at least about 40 days after administering the third population of CD45⁺ cells, and/or in an amount that maintains a target blood level of approximately 4 ng/ml or more for at least about 40 days after administering the third population of CD45⁺ cells. In some cases, the tacrolimus is administered in an amount that maintains a target blood level of at most about 10 ng/ml for at least 30 days after administering the third population of CD45⁺ cells.

In some embodiments, the tacrolimus is administered or dosed in amount to maintain or that maintains a target blood level of approximately 1 ng/mL to approximately 10 ng/mL in the human subject receiving the product for approximately 5 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 105 days or more, approximately 110 days or more, approximately 115 days or more, approximately 120 days or more, approximately 125 days or more, approximately 130 days or more, approximately 135 days or more, approximately 140 days or more, approximately 145 days or more, or approximately 150 days or more after administration of the third population of CD45⁺ cells.

In some embodiments, the tacrolimus is administered for at least about 60 days after administering the third population of CD45⁺ cells, for at least about 90 days after administering the third population of CD45⁺ cells, for at most about 150 days after administering the third population of CD45⁺ cells, for at most about 120 days after administering the third population of CD45⁺ cells.

In some embodiments, the tacrolimus is formulated for oral administration or for intravenous administration.

In embodiments, a herein-disclosed method further comprises administering to the patient over a period of time up to approximately 180 days a single graft versus host disease (GVHD) prophylactic agent (GVHDPA) comprising tacrolimus (tacrolimus GHVDPA); wherein the tacrolimus GHVDPA is administered to maintain a concentration of tacrolimus in the patient's blood above a threshold level during the period of time; and wherein a risk and/or severity of GHVD is significantly reduced. In various embodiments, the threshold level is above about 4 ng of tacrolimus per ml of patient blood or the threshold level is above about 5 ng of tacrolimus per ml of patient blood. In some embodiments, the tacrolimus GHVDPA is administered to maintain a concentration of tacrolimus in the patients' blood below an upper threshold level during the period of time. In some cases, the upper threshold level is below about 10 ng of tacrolimus per ml of patient blood.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from approximately 12 to approximately 24 hours after administration of the T-cons. In some cases, the tacrolimus GHVDPA is administered for a period of time up to approximately 90 days, is administered for a period of time up to approximately 60 days. In some embodiments, the tacrolimus GHVDPA is initially administered to the patient at approximately 0.03 mg/kg patient's actual or ideal body weight/day. In some cases, a dose of the tacrolimus GVHDPA administered to the patient is tapered starting at approximately 90 days after a first dose is administered to the patient or is tapered starting at approximately 45 days after a first dose is administered to the patient.

Tacrolimus is a macrolide that can exhibit immunosuppressive activity in vivo, and can prevent or reduce the activation of T-lymphocytes in response to antigenic or mitogenic stimulation. Tacrolimus can therefore be used to reduce the risk of GVHD in alloHSCT recipient subjects. However, methods disclosed herein—that utilize tacrolimus as a single-agent prophylactic—achieve superior clinical outcomes to those observed in other alloHSCT methods that utilize tacrolimus GVHD prophylactic. For example, in some embodiments alloHSCT methods of the disclosure that utilize tacrolimus achieve superior relapse-free survival compared to a standard of care regimen that comprises more potent GVHD prophylaxis with methotrexate plus tacrolimus, and even compared to an alloHSCT method that utilizes a drug with a similar target and mechanism of action (sirolimus). Thus, although tacrolimus can be referred to as a GVHD prophylactic herein, it can also contribute to additional therapeutic effects beyond or not directly related to GVHD prophylaxis. Therefore, as used herein, the term “tacrolimus as a single-agent GVHD prophylactic” also “includes tacrolimus as a single-agent prophylactic for additional therapeutics effects.” In other words, the term “tacrolimus as a single-agent prophylactic” and “tacrolimus as a single-agent GVHD prophylactic” are synonyms.

Treatment with tacrolimus as a single-agent prophylactic can result in, for example, decreased cytokine production and decreased T cell signal transduction. Tacrolimus can bind to the FKBP-12 protein and form a complex with calcium-dependent proteins, thereby inhibiting calcineurin phosphatase activity. This prevents or reduces the dephosphorylation and translocation of nuclear factor of activated T-cells (NFAT), a nuclear component thought to initiate gene transcription for the expression of lymphokines. Tacrolimus also inhibits the transcription for genes which encode IL-3, IL-4, IL-5, GM-CSF, and TNF, all of which are involved in the early stages of T-cell activation.

In some embodiments, tacrolimus as a single-agent prophylactic is administered to a subject orally. Absorption of tacrolimus from the gastrointestinal tract after oral administration can be incomplete and variable. The absolute bioavailability of tacrolimus in healthy subjects after oral administration can be 18±5%. The rate and extent of absorption can vary based on whether tacrolimus is given with food. In some embodiments, tacrolimus is administered parenterally, for example, intravenously or subcutaneously. In some embodiments, tacrolimus is administered by a topical, intramuscular, intradermal, intraperitoneal, intraspinal, or epidural route. Tacrolimus can be administered as an extended-release formulation. In some embodiments, tacrolimus is used as a free base. In some embodiments, tacrolimus is used as a pharmaceutically acceptable salt.

In some embodiments, methods of the disclosure can allow low doses of tacrolimus to be used. In some embodiments, a low dose of tacrolimus can improve donor T cell chimerism in a subject. In some embodiments, a low dose of tacrolimus can improve alloHSCT engraftment as disclosed herein. In some embodiments, a low dose of tacrolimus can reduce the incidence or relative risk of adverse effects that can be associated with high doses of tacrolimus, such as blurred vision, liver and kidney toxicity, seizures, tremors, hypertension, hypomagnesemia, diabetes mellitus, hyperkalemia, itching, insomnia, and confusion.

In some embodiments, the tacrolimus is initially administered to the human subject at approximately 0.03 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight/day to approximately 0.50 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight twice per day to approximately 0.50 mg/kg human subject's actual or ideal body weight twice per day, or the tacrolimus is initially administered from approximately 12 hours to approximately 24 hours after the administering of the third population of CD45⁺ cells, as disclosed herein.

A circulating level of tacrolimus can be monitored, and doses adjusted accordingly to achieve a target concentration. For example, a whole blood concentration of tacrolimus can be monitored, and doses adjusted and administered to achieve a target trough level. A target trough level of tacrolimus can be, for example, less than about 25 ng/mL, less than about 24 ng/ml, less than about 23 ng/mL, less than about 22 ng/ml, less than about 21 ng/ml, less than about 20 ng/mL, less than about 19 ng/ml, less than about 18 ng/ml, less than about 17 ng/mL, less than about 16 ng/ml, less than about 15 ng/ml, less than about 14 ng/mL, less than about 13 ng/mL, less than about 12 ng/ml, less than about 11 ng/ml, less than about 10 ng/ml, less than about 9 ng/mL, less than about 8 ng/mL, less than about 7 ng/ml, less than about 6 ng/ml, less than about 5 ng/ml, less than about 4 ng/mL, less than about 3 ng/mL, less than about 2 ng/ml, or less than about 1 ng/mL.

In some embodiments, a target trough level (e.g., trough blood level) of tacrolimus is about 1-25 ng/mL, about 1-20 ng/mL, about 1-15 ng/ml, about 1-12 ng/ml, about 1-11 ng/mL, about 1-10 ng/ml, about 1-9 ng/mL, about 1-8 ng/mL, about 1-7 ng/ml, about 1-6 ng/ml, about 1-5 ng/ml, about 1-4 ng/ml, about 1-3 ng/ml, about 1-2 ng/mL, about 2-25 ng/ml, about 2-20 ng/mL, about 2-15 ng/ml, about 2-12 ng/ml, about 2-11 ng/ml, about 2-10 ng/ml, about 2-9 ng/ml, about 2-8 ng/mL, about 2-7 ng/ml, about 2-6 ng/mL, about 2-5 ng/mL, about 2-4 ng/ml, about 2-3 ng/mL, about 3-25 ng/ml, about 3-20 ng/ml, about 3-15 ng/ml, about 3-12 ng/ml, about 3-11 ng/mL, about 3-10 ng/mL, about 3-9 ng/mL, about 3-8 ng/ml, about 3-7 ng/mL, about 3-6 ng/ml, about 3-5 ng/ml, about 3-4 ng/ml, about 4-25 ng/mL, about 4-20 ng/mL, about 4-15 ng/mL, about 4-12 ng/ml, about 4-11 ng/ml, about 4-10 ng/ml, about 4-9 ng/ml, about 4-8 ng/mL, about 4-7 ng/ml, about 4-6 ng/ml, about 4-5 ng/ml, about 5-25 ng/ml, about 5-20 ng/mL, about 5-15 ng/mL, about 5-12 ng/mL, about 5-11 ng/mL, about 5-10 ng/mL, about 5-9 ng/ml, about 5-8 ng/mL, about 5-7 ng/ml, about 5-6 ng/ml, about 6-25 g/mL, about 6-20 ng/mL, about 6-15 ng/ml, about 6-12 ng/ml, about 6-11 ng/ml, about 6-10 ng/ml, about 6-9 ng/ml, about 6-8 ng/mL, about 6-7 ng/ml, about 8-25 ng/ml, about 8-20 ng/mL, about 8-15 ng/ml, about 8-12 ng/ml, about 8-11 ng/ml, about 8-10 ng/mL, about 8-9 ng/ml, about 10-25 ng/ml, about 10-20 ng/ml, about 10-15 ng/ml, about 10-12 ng/ml, or about 10-11 ng/ml.

In some embodiments, a target trough level (e.g., trough blood level) of tacrolimus is about 6 ng/mL to approximately 10 ng/mL. In some embodiments, a target trough level of tacrolimus is about 6 ng/ml to approximately 9 ng/mL. In some embodiments, a target trough level of tacrolimus is about 6 ng/ml to approximately 8 ng/mL. In some embodiments, a target trough level of tacrolimus is about 5 ng/mL to approximately 10 ng/mL. In some embodiments, a target trough level of tacrolimus is about 5 ng/ml to approximately 9 ng/ml. In some embodiments, a target trough level of tacrolimus is about 5 ng/ml to approximately 8 ng/ml. In some embodiments, a target trough level of tacrolimus is about 4 ng/ml to approximately 10 ng/mL. In some embodiments, a target trough level of tacrolimus is about 4 ng/ml to approximately 9 ng/mL. In some embodiments, a target trough level of tacrolimus is about 4 ng/ml to approximately 8 ng/mL.

In some embodiments, a dose of tacrolimus as a single-agent prophylactic or a target trough level (e.g., trough blood level) of tacrolimus can be adjusted based on a clinical parameter disclosed herein. For example, in some cases, a dose or a target trough level of tacrolimus can be reduced if a subject exhibits lower donor T cell chimerism than desired, e.g., a percent of peripheral blood donor-derived CD3⁺ cells that is less than 95%, less than 90%, less than 85%, less than 80%, less than 75%, less than 70%, less than 65%, less than 60%, less than 55%, less than 50%, or less than 45% when evaluated after a suitable amount of time after administration of a cell population of the disclosure, for example, at approximately 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, 180 days, or 1 year after administration of a cell population of the disclosure (e.g., a first population of CD45⁺ cells). In some embodiments, a target trough level of tacrolimus can be increased if a subject exhibits signs of GVHD as disclosed herein.

In some embodiments, a subject achieves at least about 80% chimerism at approximately day 30. In some embodiments, a subject achieves at least about 80% chimerism at approximately day 30 and has a target trough level of tacrolimus of between approximately 6.5 ng/ml and approximately 9 ng/mL.

Administration of tacrolimus to a subject can commence before or after administration of a cell population disclosed herein (e.g., a first population of CD45⁺ cells). In some embodiments, administration of tacrolimus to a subject commences before administration of a cell population disclosed herein (e.g., a first population of CD45⁺ cells). In some embodiments, administration of tacrolimus to a subject commences after administration of a cell population disclosed herein (e.g., a first population of CD45⁺ cells). In some embodiments, administration of tacrolimus to a subject commences at approximately the same time as administration of a cell population disclosed herein (e.g., a first population of CD45⁺ cells).

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days before administration of a cell population disclosed herein (e.g., a population of CD45⁺ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days before administration of a cell population disclosed herein (e.g., a population of CD45⁺ cells). In some embodiments, administration of tacrolimus to a subject commences 1 day, 2 days, 3 days, 4 days, 5 days, 6 days, 7 days, 10 days, 14 days, or 20 days before administration of a cell population disclosed herein (e.g., a population of CD45⁺ cells). In some embodiments, administration of tacrolimus to a subject commences 1 day before administration of a first population of CD45⁺ cells. In some embodiments, administration of tacrolimus to a subject commences 1 day before administration of a third population of CD45⁺ cells.

In some embodiments, the tacrolimus is initially administered to the human subject at approximately 0.03 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight twice per day to approximately 0.50 mg/kg human subject's actual or ideal body weight twice per day, or the tacrolimus is initially administered from approximately 12 hours to approximately 24 hours after the administering of the third population of CD45⁺ cells, as disclosed herein.

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days after administration of a cell population disclosed herein (e.g., a population of CD45⁺ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days after administration of a cell population disclosed herein (e.g., a population of CD45⁺ cells). In some embodiments, administration of tacrolimus to a subject commences 1 day, 2 days, 3 days, 4 days, 5 days, 6 days, 7 days, 10 days, 14 days, or 20 days after administration of a cell population disclosed herein (e.g., a population of CD45⁺ cells). In some embodiments, administration of tacrolimus to a subject commences 1 day after administration of a population of CD45⁺ cells. In some embodiments, administration of tacrolimus to a subject commences 1 day after administration of a third population of CD45⁺ cells. In some embodiments, administration of tacrolimus to a subject commences the same day as a cell population of the disclosure is administered.

Tacrolimus can be administered to a subject for any amount of time after administration of a cell population of the disclosure (e.g., a population of CD45⁺ cells). In some embodiments, tacrolimus is administered to a subject for the first 7 days, 14 days, first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years after administration of a cell population of the disclosure (e.g., a population of CD45⁺ cells).

In some embodiments, tacrolimus is administered to a subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years after administration of a cell population of the disclosure (e.g., a population of CD45⁺ cells).

In various embodiments, a dose of the tacrolimus is tapered starting at approximately 90 days after the first dose is administered to the human subject or a dose of the tacrolimus is tapered starting at approximately 45 days after the first dose is administered to the human subject.

In some embodiments, a subject achieves at least about 80% chimerism at approximately day 30. In some embodiments, a subject achieves at least about 80% chimerism at approximately day 30 and has a target trough level is of approximately 6.5 ng/ml and approximately 9 ng/mL.

### Subjects

Provided herein are compositions for administration to a recipient subject having a cancer, and methods of administering the same. The compositions and methods can be useful for treating or reducing cancer in the subject. In some embodiments, a third population of CD45⁺ cells that comprises, at least, Tcons is administered to the subject in order to elicit graft-versus-tumor (GVT) immune responses and with reduced graft versus host disease (GVHD).

In some embodiments, a subject is at least 3, at least 4, at least 5, at least 6, at least 7 at least 8 at least 9 at least 10, at least 11, or at least 12 months of age. In some embodiments, a subject is at least 1, least 2, least 3, least 4, least 5, least 6, at least 7, at least 8, at least 9, at least 10, at least 11, at least 12, least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 years of age. In some embodiments, a subject is approximately 18 years of age or older. In some embodiments, a subject is approximately 16 years of age or older. In some embodiments, a subject is approximately 13 years of age or older. In some embodiments, a subject is approximately 6 years of age or older. In some embodiments, a subject is approximately 3 years of age or older. In some embodiments, a subject is approximately 1 year of age or older. In some embodiments, a subject is approximately 9 months of age or older. In some embodiments, a subject is approximately 6 months of age or older. In some embodiments, a subject is approximately 3 months of age or older.

In some embodiments, a subject is at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, or at most 80 years of age. In some embodiments, a subject is at most 65 years of age. In some embodiments, a subject is at most 70 years of age. In some embodiments, a subject is at most 75 years of age.

In some embodiments, a subject is from between approximately 3 months to approximately 18 years of age. In some embodiments, a subject is from approximately 18 years to approximately 65 years of age. In some embodiments, a subject is from approximately 18 years to approximately 75 years of age. In some embodiments, a subject is from approximately 66 years to approximately 75 years of age.

In some embodiments, the subject has received from one to five previous lines of therapy prior to administration of a composition, treatment, and/or therapy of the present disclosure. In some embodiments, the subject has received five previous lines of therapy prior to administration of a composition, treatment, and/or therapy of the present disclosure. In some embodiments, the subject has received four previous lines of therapy prior to administration of a composition, treatment, and/or therapy of the present disclosure. In some embodiments, the subject has received three previous lines of therapy prior to administration of a composition, treatment, and/or therapy of the present disclosure. In some embodiments, the subject has received two previous lines of therapy prior to administration of a composition, treatment, and/or therapy of the present disclosure. In some embodiments, the subject has received one previous line of therapy prior to administration of a composition, treatment, and/or therapy of the present disclosure. In some embodiments, the subject is or has become refractory to one or more of the previous lines of therapy prior to administration of a composition, treatment, and/or therapy of the present disclosure. In some embodiments, the subject is or has become refractory to two or more of the previous lines of therapy prior to administration of a composition, treatment, and/or therapy of the present disclosure. In some embodiments, the subject is or has become refractory to three or more of the previous lines of therapy prior to administration of a composition, treatment, and/or therapy of the present disclosure. In some embodiments, the subject is or has become refractory to four or more of the previous lines of therapy prior to administration of a composition, treatment, and/or therapy of the present disclosure. In some embodiments, the subject is or has become refractory to all previous lines of therapy prior to administration of a composition, treatment, and/or therapy of the present disclosure.

### Conditions

Another aspect provides a method of treating a human subject having (e.g., diagnosed with) or suspected of having a hematologic malignancy. The method comprises administering to the human subject a solution comprising the first population of CD45⁺ cells, a solution comprising the population of cells enriched for regulatory T cells (Tregs), a solution comprising the third population of CD45⁺ cells, and a solution comprising one or more doses of the GVHD prophylactic agent (e.g., tacrolimus). In this aspect, the solution comprising the first population of CD45⁺ cells, the solution comprising the population of cells enriched for regulatory Tregs, the solution comprising the third population of CD45⁺ cells, and the solution comprising one or more doses of the GVHD prophylactic agent are as defined according to any herein disclosed multi-component pharmaceutical treatment. Alternatively, the method comprises administering to the human subject a multi-component cellular therapy product of the present disclosure, a multi-component pharmaceutical treatment of the present disclosure, or a multi-component cellular therapy multi-component cellular therapy comprising: (a) a solution comprising a first population of isolated CD45⁺ cells comprising CD34⁺ hematopoietic stem and progenitor cells (HSPCs); (b) a solution comprising a second population of isolated CD45⁺ cells comprising fresh CD4⁺ CD25⁺ CD127^{dim} regulatory T cells (Tregs); and (c) a solution comprising a third population of isolated CD45⁺ cells comprising conventional CD3⁺ T cells (Tcons).

In some embodiments, a hematologic malignancy that may be treating by a method of the present disclosure includes, without limitation, acute leukemia in complete remission (CR), active leukemia, primary refractory acute leukemia or acute leukemia with minimal residual disease, low-risk acute myeloid leukemia (AML), high-risk acute myeloid leukemia (AML) in complete remission, chronic myelogenous leukemia (CML), high-risk myelodysplastic syndrome, therapy-related myelodysplastic syndrome and/or secondary myelodysplastic syndrome, myeloproliferative syndrome, non-Hodgkin lymphoma with poor risk features not suitable for autologous hematopoietic cell transplant (HCT), and any combinations thereof. In some embodiments, the human subject has been diagnosed with a histopathologically confirmed hematologic malignancy of the present disclosure. In some embodiments, the hematologic malignancy risk category (e.g., low risk, intermediate risk, high risk, very high risk) may be determined as per the Center for International Blood & Marrow Transplant Research (CIBMTR) Disease Risk Index (DRI).

In some embodiments, a myelodysplastic syndrome (MDS) of the present disclosure is one that is indicated for allogeneic hematopoietic cell transplant (alloHCT) per the 2017 International Expert Panel recommendations (de Witte 2017) and/or is a therapy-related/secondary MDS as defined by the World Health Organization classification of myeloid malignancies (WHO 2017).

In some embodiments, the acute leukemia is in complete remission with incomplete hematologic recovery (CRi). In any of the preceding embodiments, minimal residual disease may or may not be present in the human subject. In some embodiments, the acute leukemia is categorized as intermediate-risk to high-risk acute leukemia or very high-risk leukemia. The acute leukemia may be acute myeloid leukemia (AML), acute lymphoid leukemia (ALL), and/or mixed phenotype acute leukemia (MPAL). In some embodiments, the ALL is B cell ALL. In some embodiments, the high risk AML comprises a complex karyotype with 3 or more clonal chromosomal abnormalities. For example, 3 or more clonal chromosomal abnormalities may include monosomal karyotype-5, 5q-, -7 or 7q-, t(11q23, t(9;11), inv(3), t(3,3)t(6;9)t(9;22), normal karyotype with a fms-like tyrosine kinase 3 (FLT3)-ITD mutation, and any combination thereof. In some embodiments, the CML is in blast phase, is in second chronic phase, is in chronic phase, is accelerated, has a history of blast crisis, and/or is resistant to intolerant of one or more first-generation or second-generation tyrosine kinase inhibitors (TKIs). In some embodiments, the therapy-related myelodysplastic syndrome and/or secondary myelodysplastic syndrome is in complete remission and is categorized as intermediate-risk to high-risk.

In some embodiments, the human subject may have active disease at time of treat. In some embodiments, the human subject may have approximately 20% or less, approximately 19% or less, approximately 18% or less, approximately 17% or less, approximately 16% or less, approximately 15% or less, approximately 14% or less, approximately 13% or less, approximately 12% or less, approximately 11% or less, approximately 10% or less, approximately 9% or less, approximately 8% or less, approximately 7% or less, approximately 6% or less, or approximately 5% or less blast burden in their bone marrow.

In some embodiments, for acute myeloid leukemia and mixed phenotype acute leukemia, a complete response/complete remission (CR) may be according to FDA draft guidance for industry (“Acute Myeloid Leukemia: Developing Drugs and Biological Products for Treatment (August 2020)”. In some embodiments, a CRi may comprise meeting all CR criteria except for residual neutropenia or thrombocytopenia.

In some embodiments, for acute lymphoblastic leukemia (ALL), a CR is defined per the Center for International Blood and Marrow Transplant Research (CIBMTR 2021). In some embodiments, a CRi may comprise meeting all CR criteria except for residual neutropenia or thrombocytopenia.

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes, and a liquid suspending the cells. In this aspect, at least about 30% of the lymphocytes comprise Tcons. and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending the cells. In this aspect, at least about 30% of the lymphocytes comprise conventional T cells (Tcons) and after administration of the cell populations, the patient has a reduced risk and/or severity of the adverse event as compared to hematologic malignancy patients who received a Tcon cell population but did not receive a T-reg cell population.

Another aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

The methods of the disclosure can be used for treating a subject (e.g., a human subject) with a cancer. In some embodiments, the subject has been treated for cancer, e.g., by treatment with a chemotherapeutic drug or with radiation. The methods of the disclosure can be useful for treating a hematologic malignancy, for example, leukemia or lymphoma. Examples of hematologic malignancies that can be treated by the methods of the disclosure include, but are not limited to, acute lymphocytic leukemia (ALL), acute myelogenous leukemia (AML), chronic myelogenous leukemia (CML), multiple myeloma, lymphomas such as Hodgkin and non-Hodgkin lymphomas, myelodysplastic syndrome, myelofibrosis, and blastic plasmacytoid dendritic cell neoplasm (BPDCN). A cancer can be a solid tumor. In some embodiments, the cancer is a primary or metastatic tumor.

The types of cancer that can be treated using the methods of the present disclosure include but are not limited to leukemia, lymphoma, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and pharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, nonmelanoma skin cancers, stomach cancer, testicular cancer; thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, and Waldenstrom's macroglobulinemia.

Patients with high-risk hematologic malignancies are rarely cured with standard chemotherapy. High-risk malignancies include, for example, leukemia or lymphoma that has progressed beyond first remission, or leukemia or lymphoma with refractory relapse.

In some embodiments, the human subject or patient has previously been or is concurrently treated for the hematologic malignancy.

A subject that receives a composition of the disclosure can have, for example, acute myeloid leukemia, acute lymphoid leukemia, mixed phenotype leukemia, myelofibrosis, high-risk myelodysplastic syndrome, very high-risk myelodysplastic syndrome, myelofibrosis (MF) that is eligible for transplant per National Comprehensive Cancer Network Guidelines, intermediate-2- or high-risk MF according to the IPSS, DIPSS or DIPSS-plus scoring systems, intermediate-1-risk MF associated with high-risk features such as high symptoms burden, low platelet counts, or complex cytogenetics, primary myelofibrosis, myelofibrosis evolved from another myeloproliferative neoplasm, myelodysplastic syndrome, non-Hodgkin lymphoma, a non-malignant indication for allogeneic hematopoietic stem cell transplantation (alloHSCT),

In some embodiments, a subject has acute myeloid leukemia. In some embodiments, a subject has acute lymphoid leukemia. In some embodiments, a subject has mixed phenotype leukemia. In some embodiments, a subject has high-risk myelodysplastic syndrome. In some embodiments, a subject has very high-risk myelodysplastic syndrome. In some embodiments, a subject has myelofibrosis (MF) that is eligible for transplant per National Comprehensive Cancer Network Guidelines. In some embodiments, a subject has intermediate-2- or high-risk myelofibrosis according to the IPSS, DIPSS or DIPSS-plus scoring systems. In some embodiments, a subject has intermediate-1-risk myelofibrosis associated with high-risk features such as high symptoms burden, low platelet counts, or complex cytogenetics. In some embodiments, a subject has primary myelofibrosis. In some embodiments, a subject has myelofibrosis. In some embodiments, a subject has myelofibrosis evolved from another myeloproliferative neoplasm. In some embodiments, a subject has myelodysplastic syndrome. In some embodiments, a subject has non-Hodgkin lymphoma. In some embodiments, a subject has a non-malignant indication for alloHSCT. In some embodiments, a subject has blastic plasmacytoid dendritic cell neoplasm (BPDCN).

A subject can be in complete remission (CR). A subject can be in complete remission with incomplete hematologic recovery (CRi), e.g., without the presence of known minimal residual disease. A subject can have minimal residual disease. A subject can have no evidence of minimal residual disease. A subject can have active disease. A subject can have a leukemia (e.g., acute myeloid, acute lymphoid, or mixed phenotype) that is not in morphologic CR with bone marrow infiltration by leukemic blasts of ≤10%. A subject can have a leukemia (e.g., acute myeloid, acute lymphoid, or mixed phenotype) that is in morphologic CR with evidence of minimal residual positivity by either multiparameter flow cytometric analysis or by a nucleic acid-based technique.

Complete remission (CR) for acute myeloid, lymphoid or mixed phenotype leukemia can be indicated by meeting all of the following criteria: (i) Bone marrow blasts <5%; (ii) Absence of circulating blasts and blasts with Auer rods; (ii) Absence of extramedullary disease 4. ANC≥1.0×10⁹/L (1,000/μL); (iii) Platelet count≥100×10⁹/L (100,000/μL); and (iv) Independence of red cell transfusions. Complete Response with Incomplete Hematologic Recovery (CRi) can be indicated by meeting all the CR criteria except for residual neutropenia (<1.0×10⁹/L) or thrombocytopenia (<100×10⁹/L).

Another aspect provides methods for treating a human subject having (e.g., diagnosed) or suspected of having multiple sclerosis with any of the compositions, treatments, or cellular therapy products of the present disclosure. In some embodiments, the multiple sclerosis is primary progressive multiple sclerosis, chronic progressive multiple sclerosis, secondary progressive multiple sclerosis, relapsing-remitting multiple sclerosis, acute relapsing multiple sclerosis, or any combination thereof.

In some embodiments, the methods of the present disclosure may further include administering one or more additional therapeutic agents. Examples, of such therapeutic agents include, without limitation, an inhibitor of an immunosuppressive enzymes, e.g., narginase II and indoleamine 2,3-dioxygenase 1 (IDO1); a proinflammatory cytokine, e.g., IL-2 or IL-15; a blocker of an anti-inflammatory cytokine, e.g., an antibody that binds to and/or blocks M-CSF, IL-4, or TGF-beta; a bispecific antibody, e.g., that targets a protein or proteins relating to Natural Killer (NK) function, such as an NKG2D-based or CD16-based bispecific antibody; a check-point inhibitor, e.g., a CTLA4 inhibitor, a PD1 inhibitor, a PD-L1 inhibitor, a CD47/SIRP-alpha inhibitor, a LAG-3 inhibitor, a TIM-3 inhibitor, a B7-H3 inhibitor, a B7-H4 inhibitor, an A2aR inhibitor, a CD73 inhibitor, an NKG2A inhibitor, a PVRIG/PVRL2 inhibitor, a CEACAM1 inhibitor, a CEACAM 5/6 inhibitor, a FAK inhibitor, a CCL2/CCR2 inhibitor, a LIF inhibitor, a CSF-1 inhibitor, an IL-8 inhibitor, a SEMA4D inhibitor, an Ang-2 inhibitor, a CLEVER-1 inhibitor, an Axl inhibitor, a phsphatidylserine inhibitor, and/or a TIGIT inhibitor; a JAK/STAT inhibitor, e.g., a Jak2 inhibitor; a FLT3 inhibitor; and one or more CD45⁺ cells, e.g., one or more T cells, NK cells, CTLs, Tregs, or NKT cells, comprising one or more chimeric receptors. In some embodiments, the chimeric receptor comprises one or more extracellular antigen-binding domains that bind to one or more cancer-associated antigens. Any suitable cancer-associated antigen known in the art may be targeted. In some embodiments, the chimeric receptor is a chimeric antigen receptor or a T cell receptor.

### Sensitivities

In some embodiments, a subject does not have a known allergy or hypersensitivity to, or intolerance of, tacrolimus. In some embodiments, a subject does not have a known allergy or hypersensitivity to, or intolerance of, sirolimus.

In some embodiments, subjects are not sensitive to iron dextran (e.g., subjects with sensitivity to iron dextran are not eligible to receive a composition of the disclosure. In some cases, this may be because of the magnetic beads used in some embodiments to isolate, deplete, and/or purify cell types).

In some embodiments, subjects are not sensitive to products derived from cyanine dyes (e.g., subjects with sensitivity to products derived from cyanine dyes are not eligible to receive a composition of the disclosure).

In some embodiments, subjects are not sensitive to proteins products derived from murine sources (e.g., subjects with sensitivity to proteins products derived from murine sources are not eligible to receive a composition of the disclosure).

In some embodiments, subjects are not sensitive to proteins products derived from bovine sources (e.g., subjects with sensitivity to proteins products derived from bovine sources are not eligible to receive a composition of the disclosure).

In some embodiments, subjects are not sensitive to proteins products derived from algal sources (e.g., subjects with sensitivity to proteins products derived from algal sources are not eligible to receive a composition of the disclosure).

In some embodiments, subjects are not sensitive to proteins products derived from Streptomyces avidinii (e.g., subjects with sensitivity to proteins products derived from Streptomyces avidinii are not eligible to receive a composition of the disclosure).

### Organ Function and Biomarkers

A subject can have an estimated glomerular filtration rate (eGFR)>30 mL/minute. A subject can have an estimated glomerular filtration rate (eGFR)>40 mL/minute. A subject can have an eGCF of >50 mL/minute. A subject can have an estimated glomerular filtration rate (eGFR)>60 mL/minute.

A subject can have a cardiac ejection fraction at rest ≥45%, or shortening fraction of ≥27% by echocardiogram or radionuclide scan (MUGA).

A subject can have a diffusing capacity of the lung for carbon monoxide (DLCO) (adjusted for hemoglobin) of ≥50%.

A subject can have a negative serum or urine beta-HCG test, e.g., in females of childbearing potential within 3 weeks of registration.

A subject can have total bilirubin <2 times upper limit of normal (ULN).

A subject can have Gilbert's syndrome, wherein hemolysis has been excluded.

A subject can have an ALT reading within 3 times upper limit of normal (ULN). A subject can have an AST reading within 3 times upper limit of normal (ULN).

### Additional Therapies and Other Subject Characteristics

In some embodiments, a subject has not received a prior alloHSCT. In some embodiments, a subject is not a candidate for autologous transplant. In some embodiments, a subject is not receiving corticosteroids or other immunosuppressive therapy. In some embodiments, a subject is receiving topical corticosteroids or oral systemic corticosteroid doses less than or equal to 10 mg/day. In some embodiments, a subject does not receive donor lymphocyte infusion (DLI). In some embodiments, a subject does not receive a T cell depleting pharmaceutical, e.g., post-transplant cyclophosphamide (Cy), peri-transplant anti-thymocyte globulin (ATG), or alemtuzumab. In some embodiments, a subject that has previously been exposed to a T cell-depleting agent has a 5-half-life washout of the agent prior to planned transplant day 0 (day of infusion of the Treg and HSPC components of the graft). In some embodiments, a subject is not positive for anti-donor HLA antibodies against a mismatched allele in the selected donor as determined by either: (a) A positive crossmatch test of any titer; or (b) The presence of anti-donor HLA antibody to any HLA locus. In some embodiments, the subject has a Karnofsky performance score ≥70%. In some embodiments, a subject does not have a hematopoietic cell transplantation-specific Comorbidity Index (HCT-CI) of >4. In some embodiments, a subject does not have an uncontrolled bacterial, viral, or fungal infection. In some embodiments, a subject is not taking antimicrobial therapy and with progression or no clinical improvement in infection. In some embodiments, a subject is not seropositive for HIV-1 or -2, HTLV-1 or -2, Hepatitis B sAg, or Hepatitis C antibody. In some embodiments, a subject does not have an uncontrolled autoimmune disease that requires active immunosuppressive treatment. In some embodiments, a subject does has not had concurrent malignancies or active disease within 1 year, for example, excluding non-melanoma skin cancers that have been curatively resected. In some embodiments, a subject does not exhibit psychosocial circumstances that preclude the patient being able to go through transplant or participate responsibly in follow up care. In some embodiments, a subject is not pregnant or breastfeeding. In some embodiments, a subject does not have a serious medical condition or abnormality in clinical laboratory tests that, in the medical professional's judgment, precludes the subject's safety upon receipt of a composition of the disclosure. In some embodiments, a subject is eligible for myeloablative alloHSCT.

In some embodiments, a subject receives a prophylactic agent to reduce the risk of bacterial, fungal, and/or viral infection, e.g., during the peri-transplant period.

In some embodiments, a subject receives a supportive therapy for HCT-related toxicity. In some embodiments, a subject does not receive a supportive therapy for HCT-related toxicity. In some embodiments, a subject receives a growth factor. In some embodiments, a subject does not receive a growth factor. In some embodiments, a subject receives intravenous immunoglobulin. In some embodiments, a subject does not receive intravenous immunoglobulin. In some embodiments, a subject receives an analgesic. In some embodiments, a subject does not receive an analgesic. In some embodiments, a subject receives an anti-emetic. In some embodiments, a subject does not receive an anti-emetic. In some embodiments, a subject receives electrolyte replacement. In some embodiments, a subject does not receive electrolyte replacement. In some embodiments, a subject receives a tyrosine kinase inhibitor (e.g., a FLT3 inhibitor). In some embodiments, a subject does not receive a tyrosine kinase inhibitor (e.g., a FLT3 inhibitor). In some embodiments, a subject receives prednisone or an equivalent thereof, e.g., at a dose of ≤ 10 mg/day. In some embodiments, a subject does not receive prednisone or an equivalent thereof. In some embodiments, a subject receives corticosteroid treatment to manage GVHD. In some embodiments, a subject does not receive corticosteroid treatment to manage GVHD. In some embodiments, a subject receives high-dose corticosteroid treatment to manage GVHD. In some embodiments, a subject does not receive high-dose corticosteroid treatment to manage GVHD. In some embodiments, a subject receives corticosteroid treatment to manage, for example, adrenal insufficiency, hypersensitivity reactions, or other non-cancer-related symptoms including premedication for known hypersensitivity reactions to contrast for scans. In some embodiments, a subject does not receive corticosteroid treatment. In some embodiments, a subject receives an immunosuppressive medication. In some embodiments, a subject does not receive an immunosuppressive medication. In some embodiments, a subject receives a donor lymphocyte infusion. In some embodiments, a subject does not receive a donor lymphocyte infusion.

### Conditioning Regimens

Conditioning regimens can be used as part of an alloHSCT regimen of the disclosure. Chemotherapy and/or irradiation given soon before a transplant is called a conditioning regimen. Conditioning regimens can help eradicate a patient's disease prior to the infusion of HSPCs, suppress immune reactions, and allow a donor HSPCs to reconstitute the vacant hematopoietic compartment that results from the conditioning regimen. In some embodiments of the methods of the disclosure, a subject can be treated with myeloablative conditioning prior to infusion of cell populations described herein. In some embodiments of the methods of the disclosure, a subject can be treated with myeloreductive conditioning prior to infusion of cell populations described herein. In some embodiments of the methods of the disclosure, a subject can be treated with a reduced intensity myeloablative conditioning prior to infusion of cell populations described herein. In some embodiments of the methods of the disclosure, a subject can be treated with non-myeloablative conditioning prior to administering a cell population or cell populations described herein.

As used herein, the term conditioning regimen and the like applies to myeloablative conditioning, myeloreductive conditioning, reduced intensity myeloablative therapy/conditioning, and/or non-myeloablative conditioning. As used herein, the term myeloablative therapy/conditioning also includes myeloreductive conditioning and reduced intensity myeloablative conditioning.

In aspects and embodiments, a treatment and/or method further comprises a conditioning regimen, wherein the conditioning regimen is administered before administration of one of (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); and (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent. In aspects and embodiments, a treatment and/or method further comprises a conditioning regimen, wherein the conditioning regimen is administered before administration of (a) a solution comprising a first population of isolated CD45⁺ cells comprising CD34⁺ hematopoietic stem and progenitor cells (HSPCs); (b) a solution comprising a second population of isolated CD45⁺ cells comprising fresh CD4⁺CD25⁺CD127^{dim} regulatory T cells (Tregs); and (c) a solution comprising a third population of isolated CD45⁺ cells comprising conventional CD3⁺ T cells (Tcons).

In embodiments, the conditioning regimen is a myeloablative conditioning regimen. In some cases, the conditioning regimen comprises at least three conditioning reagents, wherein at least one conditioning reagent is thiotepa. In various embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In some embodiments, the conditioning regimen comprises one or more doses of busulfan, fludarabine and thiotepa. In embodiments, the one or more doses comprises from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.

In various embodiments, the method further comprises administering a myeloablative conditioning regimen to the patient prior to the administration of any cell population, the conditioning regimen comprising administration of at least one conditioning agent to the patient.

In some embodiments, the patient does not receive any irradiation as part of the myeloablative conditioning regimen.

In embodiments, the at least one conditioning agent is administered from approximately two to approximately ten days prior to the administration of any of the cell populations. In some cases, the at least one conditioning agent is administered about five days prior to the administration of any of the cell populations.

In various embodiments, the human subject has undergone myeloablative conditioning regimen before administration of any cell populations and the adverse event is associated with the myeloablative conditioning.

In some embodiments, the at least one conditioning agent comprises thiotepa. In some cases, a dose of thiotepa administered to the patient is in a range of from approximately 5 to approximately 10 mg per kilogram of actual or ideal body weight.

In embodiments, the at least one conditioning agent comprises busulfan and fludarabine. In some cases, doses of thiotepa, busulfan, and fludarabine administered to the patient comprise about 10 mg per kilogram of the patient's actual or ideal body weight, about 9.6 mg per kilogram of the patient's actual or ideal body weight, and approximately 150 mg per meter² body surface area respectively.

In some embodiments, the subject has been conditioned with radiation, chemotherapy, recombinant proteins, antibodies, or toxin-conjugated antibodies, or any combination thereof prior to administering a cell population or cell populations described herein. In some embodiments, the subject is conditioned for cellular graft therapy by first treating the subject with myeloablative therapy. Exemplary myeloablative therapies include chemotherapy or radiotherapy. Myeloablative therapies are thought to provide therapeutic benefit by debulking a tumor and/or reducing the number of cancer cells. Myeloablative regimens eradicate a sufficient number of HSCs that the patient would otherwise increase the chances of a patient developing GVHD. When HSPCs are subsequently administered to the myeloablated subject, the donor cells can further attack the cancer and/or and reconstitute the blood and the immune system of the subject.

In some embodiments, the myeloablative therapy comprises administration of thiotepa (TTP), busulfan, cyclophosphamide, Total Body Irradiation (TBI), fludarabine, etoposide, melphalan, anti-thymocyte globulin (ATG), or any combination thereof. In some embodiments, the myeloablative therapy comprises administration an anti-cKIT antibody. In some embodiments, the myeloablative therapy comprises administration an antibody drug conjugate. The antibody drug conjugate can be, for example, anti-CD45-saporin or anti-cKit-saporin therapeutic antibodies. In some embodiments, the myeloablative therapy is a reduced intensity conditioning therapy. Exemplary conditioning regimens are described in Table 15.

A conditioning regimen of this disclosure may comprise one or more doses of busulfan. A conditioning regimen of this disclosure may comprise fludarabine. A conditioning regimen of this disclosure may comprise one or more doses of Cyclophosphamide. A conditioning regimen of this disclosure may comprise one or more doses of Melphalan. A conditioning regimen of this disclosure may comprise one or more doses of Etoposide.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

The conditioning regimen described herein may comprise administration of an alkylating agent such as thiotepa (TTP). A conditioning regimen of this disclosure comprising TTP may comprise at least one more conditioning reagent. The conditioning reagents administered to a subject in addition to TTP may comprise one or more reagents selected from busulfan, dimethyl myleran, prednisone, methyl prednisolone, azathioprine, cyclophosphamide, cyclosparine, monoclonal antibodies against T cells, antilymphocyte globulin and anti-thymocyte globulin, fludarabine, etoposide, radiation, total body irradiation (TBI). Aspects and embodiments include any combination of TTP with the one or more conditioning reagents. In some embodiments, a subject is administered a conditioning regimen comprising thiotepa, busulfan, and fludarabine. In some embodiments, a subject is administered a conditioning regimen comprising thiotepa, fludarabine, and TBI (e.g., HFTBI).

The conditioning regimen described herein may comprise administration of an alkylating agent such as TTP. In some cases, a conditioning regimen of the disclosure may comprise TTP administration on more than one day. A conditioning regimen of the disclosure may comprise administering 2 mg/kg to 14 mg/kg TTP to a subject. A conditioning regimen of this disclosure may comprise administering at least 3 mg/kg TTP to a subject. A conditioning regimen of this disclosure may comprise administering at most 14 mg/kg TTP to a subject. A conditioning regimen of this disclosure may comprise administering 2 mg/kg to 5 mg/kg, 2 mg/kg to 6 mg/kg, 2 mg/kg to 8 mg/kg, 2 mg/kg to 10 mg/kg, 2 mg/kg to 12 mg/kg, 2 mg/kg to 14 mg/kg, 5 mg/kg to 6 mg/kg, 5 mg/kg to 8 mg/kg, 5 mg/kg to 10 mg/kg, 5 mg/kg to 12 mg/kg, 5 mg/kg to 14 mg/kg, 6 mg/kg to 8 mg/kg, 6 mg/kg to 10 mg/kg, 6 mg/kg to 12 mg/kg, 6 mg/kg to 14 mg/kg, 8 mg/kg to 10 mg/kg, 8 mg/kg to 12 mg/kg, 8 mg/kg to 14 mg/kg, 10 mg/kg to 12 mg/kg, 10 mg/kg to 14 mg/kg, or 12 mg/kg to 14 mg/kg TTP to a subject. A conditioning regimen of this disclosure may comprise administering 2 mg/kg, 3 mg/kg, 4 mg/kg, 5 mg/kg, 6 mg/kg, 8 mg/kg, 10 mg/kg, 12 mg/kg, or 14 mg/kg TTP to a subject. A conditioning regimen of this disclosure may comprise administering at most 2 mg/kg, 3 mg/kg, 4 mg/kg, 5 mg/kg, 6 mg/kg, 8 mg/kg, 10 mg/kg, or 12 mg/kg TTP to a subject. A conditioning regimen of this disclosure may comprise administering at least 2 mg/kg, 3 mg/kg, 4 mg/kg, 5 mg/kg, 6 mg/kg, 8 mg/kg, 10 mg/kg, 12 mg/kg, or 14 mg/kg TTP to a subject.

As used herein, a recited dose, e.g., #mg/kg, may be relative to a subject's actual body weight (in kg) or relative to the subject's ideal body weight (in kg). Or the recited dose may be relative to a subject's adjusted body weight (ABW) if the subject's actual body weight is greater than 120% of the ideal body weight (IBW).

A subject administered one or more cell populations described herein may be administered one or more doses of TTP prior to the cell transplant. A subject receiving one or more cell populations described herein may be administered 1, 2, 3, 4, 5, 6, 7, 8, 9 or 10 doses of TTP prior to the cell transplant. In some cases, each dose of TTP has the same concentration. In some cases, one or more doses of TTP have different concentrations. A subject may be administered 1 mg/kg to 10 mg/kg TTP in a single dose. A subject may be administered at least 1 mg/kg TTP in a single dose. A subject may be administered at most 10 mg/kg TTP in a single dose. A subject may be administered 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 1 mg/kg to 10 mg/kg, 2 mg/kg to 3 mg/kg, 2 mg/kg to 4 mg/kg, 2 mg/kg to 5 mg/kg, 2 mg/kg to 6 mg/kg, 2 mg/kg to 7 mg/kg, 2 mg/kg to 8 mg/kg, 2 mg/kg to 9 mg/kg, 2 mg/kg to 10 mg/kg, 3 mg/kg to 4 mg/kg, 3 mg/kg to 5 mg/kg, 3 mg/kg to 6 mg/kg, 3 mg/kg to 7 mg/kg, 3 mg/kg to 8 mg/kg, 3 mg/kg to 9 mg/kg, 3 mg/kg to 10 mg/kg, 4 mg/kg to 5 mg/kg, 4 mg/kg to 6 mg/kg, 4 mg/kg to 7 mg/kg, 4 mg/kg to 8 mg/kg, 4 mg/kg to 9 mg/kg, 4 mg/kg to 10 mg/kg, 5 mg/kg to 6 mg/kg, 5 mg/kg to 7 mg/kg, 5 mg/kg to 8 mg/kg, 5 mg/kg to 9 mg/kg, 5 mg/kg to 10 mg/kg, 6 mg/kg to 7 mg/kg, 6 mg/kg to 8 mg/kg, 6 mg/kg to 9 mg/kg, 6 mg/kg to 10 mg/kg, 7 mg/kg to 8 mg/kg, 7 mg/kg to 9 mg/kg, 7 mg/kg to 10 mg/kg, 8 mg/kg to 9 mg/kg, 8 mg/kg to 10 mg/kg, or 9 mg/kg to 10 mg/kg TTP in a single dose. A subject may be administered 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at most 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at least 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

A conditioning regimen of this disclosure may comprise one or more doses of busulfan. One or more doses of busulfan may be administered to a subject before the administration of one or more doses of another conditioning reagent such as TTP. One or more doses of busulfan may be administered to a subject after the administration of one or more doses of another conditioning reagent such as TTP. One or more doses of busulfan may be administered to a subject along with the administration of one or more doses of another conditioning reagent such as TTP.

A conditioning regimen of this disclosure may comprise administering about 6 mg/kg to approximately 12 mg/kg busulfan to a subject. A conditioning regimen of this disclosure may comprise administering at least about 6 mg/kg busulfan to a subject. A conditioning regimen of this disclosure may comprise administering at most about 12 mg/kg busulfan to a subject. A conditioning regimen of this disclosure may comprise administering about 6 mg/kg to approximately 7 mg/kg, about 6 mg/kg to approximately 8 mg/kg, about 6 mg/kg to approximately 9 mg/kg, about 6 mg/kg to approximately 10 mg/kg, about 6 mg/kg to approximately 11 mg/kg, about 6 mg/kg to approximately 12 mg/kg, about 7 mg/kg to approximately 8 mg/kg, about 7 mg/kg to approximately 9 mg/kg, about 7 mg/kg to approximately 10 mg/kg, about 7 mg/kg to approximately 11 mg/kg, about 7 mg/kg to approximately 12 mg/kg, about 8 mg/kg to approximately 9 mg/kg, about 8 mg/kg to approximately 10 mg/kg, about 8 mg/kg to approximately 11 mg/kg, about 8 mg/kg to approximately 12 mg/kg, about 9 mg/kg to approximately 10 mg/kg, about 9 mg/kg to approximately 11 mg/kg, about 9 mg/kg to approximately 12 mg/kg, about 10 mg/kg to approximately 11 mg/kg, about 10 mg/kg to approximately 12 mg/kg, or about 11 mg/kg to approximately 12 mg/kg busulfan to a subject. A conditioning regimen of this disclosure may comprise administering 6 mg/kg, 7 mg/kg, 8 mg/kg, 9 mg/kg, 10 mg/kg, 11 mg/kg, or 12 mg/kg busulfan to a subject. A conditioning regimen of this disclosure may comprise administering at least 6 mg/kg, 7 mg/kg, 8 mg/kg, 9 mg/kg, 10 mg/kg, or 11 mg/kg busulfan to a subject. A conditioning regimen of this disclosure may comprise administering at most 7 mg/kg, 8 mg/kg, 9 mg/kg, 10 mg/kg, 11 mg/kg, or 12 mg/kg busulfan to a subject.

A subject receiving one or more cell populations described herein may be administered one or more doses of busulfan prior to the cell transplant. A subject receiving one or more cell components described herein may be administered 1, 2, 3, 4, 5, 6, 7, 8, 9 or 10 doses of busulfan prior to the cell transplant. In some cases, each dose of busulfan has the same concentration. In some cases, one or more doses of busulfan have different concentrations. A subject may be administered 1 mg/kg to 10 mg/kg busulfan in a single dose. A subject may be administered at least 1 mg/kg busulfan in a single dose. A subject may be administered at least 2 mg/kg busulfan in a single dose. A subject may be administered at least 3 mg/kg busulfan in a single dose.

A conditioning regimen of this disclosure may comprise one or more doses of fludarabine. One or more doses of fludarabine may be administered to a subject before the administration of one or more doses of another conditioning reagent such as TTP. One or more doses of fludarabine may be administered to a subject after the administration of one or more doses of another conditioning reagent such as TTP. One or more doses of fludarabine may be administered to a subject along with the administration of one or more doses of another conditioning reagent such as TTP.

A conditioning regimen of this disclosure may comprise administering 20 mg/m² to 180 mg/m² fludarabine to a subject based on the surface area of the subject. A conditioning regimen of this disclosure may comprise administering at least 20 mg/m² fludarabine to a subject. A conditioning regimen of this disclosure may comprise administering at most 180 mg/m² fludarabine to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² fludarabine to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² fludarabine to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² fludarabine to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² fludarabine to a subject.

A subject receiving one or more cell components described herein may be administered one or more doses of fludarabine prior to the cell transplant. A subject receiving one or more cell components described herein may be administered 1, 2, 3, 4, 5, 6, 7, 8, 9 or 10 doses of fludarabine prior to the cell transplant. In some cases, each dose of fludarabine has the same concentration. In some cases, one or more doses of fludarabine have different concentrations. A subject may be administered 20 to 60 mg/m² dose of fludarabine in a single dose. A subject may be administered at least 30 mg/m² fludarabine in a single dose. A subject may be administered at least 40 mg/m² fludarabine in a single dose. A subject may be administered at least 50 mg/m² fludarabine in a single dose.

A conditioning regimen of this disclosure may comprise administering 20 mg/m² to 180 mg/m² melphalan to a subject based on the surface area of the subject. A conditioning regimen of this disclosure may comprise administering at least 20 mg/m² melphalan to a subject. A conditioning regimen of this disclosure may comprise administering at most 180 mg/m² melphalan to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² melphalan to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² melphalan to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² melphalan to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² melphalan to a subject.

A subject receiving one or more cell components described herein may be administered one or more doses of melphalan prior to the cell transplant. A subject receiving one or more cell components described herein may be administered 1, 2, 3, 4, 5, 6, 7, 8, 9 or 10 doses of melphalan prior to the cell transplant. In some cases, each dose of melphalan has the same concentration. In some cases, one or more doses of melphalan have different concentrations.

A conditioning regimen of this disclosure may comprise administering 100 mg/kg to 140 mg/kg cyclophosphamide. A conditioning regimen of this disclosure may comprise administering at least 100 mg/kg cyclophosphamide. A conditioning regimen of this disclosure may comprise administering at most 140 mg/kg cyclophosphamide. A conditioning regimen of this disclosure may comprise administering 100 mg/kg to 110 mg/kg, 100 mg/kg to 120 mg/kg, 100 mg/kg to 130 mg/kg, 100 mg/kg to 140 mg/kg, 110 mg/kg to 120 mg/kg, 110 mg/kg to 130 mg/kg, 110 mg/kg to 140 mg/kg, 120 mg/kg to 130 mg/kg, 120 mg/kg to 140 mg/kg, or 130 mg/kg to 140 mg/kg cyclophosphamide. A conditioning regimen of this disclosure may comprise administering about 100 mg/kg, 110 mg/kg, 120 mg/kg, 130 mg/kg, or 140 mg/kg cyclophosphamide. A conditioning regimen of this disclosure may comprise administering at least 100 mg/kg, 110 mg/kg, 120 mg/kg, or 130 mg/kg cyclophosphamide. A conditioning regimen of this disclosure may comprise administering at most 110 mg/kg, 120 mg/kg, 130 mg/kg, or 140 mg/kg cyclophosphamide.

A subject receiving one or more cell components described herein may be administered one or more doses of cyclophosphamide prior to the cell transplant. A subject receiving one or more cell components described herein may be administered 1, 2, 3, 4, 5, 6, 7, 8, 9 or 10 doses of cyclophosphamide prior to the cell transplant. In some cases, each dose of cyclophosphamide has the same concentration. In some cases, one or more doses of cyclophosphamide have different concentrations.

A conditioning regimen of this disclosure may comprise administering 40 mg/kg to 80 mg/kg etoposide. A conditioning regimen of this disclosure may comprise administering at least 40 mg/kg etoposide. A conditioning regimen of this disclosure may comprise administering at most 80 mg/kg etoposide. A conditioning regimen of this disclosure may comprise administering 40 mg/kg to 50 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, 50 mg/kg to 80 mg/kg, 60 mg/kg to 70 mg/kg, 60 mg/kg to 80 mg/kg, or 70 mg/kg to 80 mg/kg etoposide. A conditioning regimen of this disclosure may comprise administering about 40 mg/kg, 50 mg/kg, 60 mg/kg, 70 mg/kg, or 80 mg/kg etoposide. A conditioning regimen of this disclosure may comprise administering at least 40 mg/kg, 50 mg/kg, 60 mg/kg, or 70 mg/kg etoposide. A conditioning regimen of this disclosure may comprise administering at most 50 mg/kg, 60 mg/kg, 70 mg/kg, or 80 mg/kg etoposide.

A subject receiving one or more cell components described herein may be administered one or more doses of etoposide prior to the cell transplant. A subject receiving one or more cell components described herein may be administered 1, 2, 3, 4, 5, 6, 7, 8, 9 or 10 doses of etoposide prior to the cell transplant. In some cases, each dose of etoposide has the same concentration. In some cases, one or more doses of etoposide have different concentrations.

A conditioning regimen of this disclosure comprising TTP may comprise one or more doses of total body irradiation (TBI) such as hyperfractionated TBI (HFTBI). One or more doses of HFTBI may be administered to a subject before the administration of one or more doses of another conditioning reagent such as TTP. One or more doses of HFTBI may be administered to a subject after the administration of one or more doses of another conditioning reagent such as TTP. One or more doses of HFTBI may be administered to a subject along with the administration of one or more doses of another conditioning reagent such as TTP.

A conditioning regimen of this disclosure may comprise administering 800 cGy to 1,500 cGy HFTBI to a subject. A conditioning regimen of this disclosure may comprise administering at least 800 cGy HFTBI to a subject. A conditioning regimen of this disclosure may comprise administering at most 1,500 cGy HFTBI to a subject. A conditioning regimen of this disclosure may comprise administering 800 cGy to 900 cGy, 800 cGy to 1,000 cGy, 800 cGy to 1,100 cGy, 800 cGy to 1,200 cGy, 800 cGy to 1,300 cGy, 800 cGy to 1,375 cGy, 800 cGy to 1,400 cGy, 800 cGy to 1,500 cGy, 900 cGy to 1,000 cGy, 900 cGy to 1,100 cGy, 900 cGy to 1,200 cGy, 900 cGy to 1,300 cGy, 900 cGy to 1,375 cGy, 900 cGy to 1,400 cGy, 900 cGy to 1,500 cGy, 1,000 cGy to 1,100 cGy, 1,000 cGy to 1,200 cGy, 1,000 cGy to 1,300 cGy, 1,000 cGy to 1,375 cGy, 1,000 cGy to 1,400 cGy, 1,000 cGy to 1,500 cGy, 1,100 cGy to 1,200 cGy, 1,100 cGy to 1,300 cGy, 1,100 cGy to 1,375 cGy, 1,100 cGy to 1,400 cGy, 1,100 cGy to 1,500 cGy, 1,200 cGy to 1,300 cGy, 1,200 cGy to 1,375 cGy, 1,200 cGy to 1,400 cGy, 1,200 cGy to 1,500 cGy, 1,300 cGy to 1,375 cGy, 1,300 cGy to 1,400 cGy, 1,300 cGy to 1,500 cGy, 1,375 cGy to 1,400 cGy, 1,375 cGy to 1,500 cGy, or 1,400 cGy to 1,500 cGy HFTBI to a subject. A conditioning regimen of this disclosure may comprise administering about 800 cGy, 900 cGy, 1,000 cGy, 1,100 cGy, 1,200 cGy, 1,300 cGy, 1,375 cGy, 1,400 cGy, or 1,500 cGy HFTBI to a subject. A conditioning regimen of this disclosure may comprise administering at least 800 cGy, 900 cGy, 1,000 cGy, 1,100 cGy, 1,200 cGy, 1,300 cGy, 1,375 cGy or 1,400 cGy HFTBI to a subject. A conditioning regimen of this disclosure may comprise administering at most 900 cGy, 1,000 cGy, 1,100 cGy, 1,200 cGy, 1,300 cGy, 1,375 cGy, 1,400 cGy, or 1,500 cGy HFTBI to a subject.

A subject receiving one or more cell components described herein may be administered one or more doses of HFTBI prior to the cell transplant. A subject receiving one or more cell components described herein may be administered 1, 2, 3, 4, 5, 6, 7, 8, 9 or 10 doses of HFTBI prior to the cell transplant. In some cases, each dose of HFTBI has the same concentration. In some cases, one or more doses of HFTBI have different concentrations. A subject may be administered a 75 to 150 cGys of HFTBI in a single dose. A subject may be administered at least 75 cGys HFTBI in a single dose. A subject may be administered at least 100 cGys HFTBI in a single dose. A subject may be administered at least 125 cGys HFTBI in a single dose.
Exemplary Conditioning Regimen 1: In some embodiments, a subject is administered a conditioning regimen comprising thiotepa, busulfan, and fludarabine. In some embodiments, a subject is administered thiotepa at 5 mg/kg actual body weight or ideal body weight. In some embodiments, a subject is administered thiotepa at approximately 5 mg/kg for two days (e.g., consecutive days). In some embodiments, a subject is administered busulfan at approximately 3.2 mg/kg actual body weight or ideal body weight. In some embodiments, a subject is administered busulfan at approximately 3.2 mg/kg daily for three days (e.g., consecutive days). In some embodiments, a subject is administered fludarabine at approximately 50 mg/m². (meters squared−body surface area). In some embodiments, a subject is administered fludarabine at approximately 50 mg/m² for three days (e.g., consecutive days). In some embodiments, a subject is administered thiotepa at 5 mg/kg actual body weight or ideal body weight, is administered busulfan at approximately 3.2 mg/kg actual body weight or ideal body weight, and is administered fludarabine at approximately 50 mg/m². (meters squared-body surface area). In some embodiments, a subject is administered thiotepa at approximately 5 mg/kg for two days (e.g., consecutive days), is administered busulfan at approximately 3.2 mg/kg daily for three days (e.g., consecutive days), and is administered fludarabine at approximately 50 mg/m² for three days (e.g., consecutive days). In some embodiments, a subject is administered thiotepa at approximately 5 mg/kg on days −7 and −6, is administered busulfan at approximately 3.2 mg/kg daily on days −5 to −3, and is administered fludarabine at approximately 50 mg/m² on days −5 to −3.
Exemplary Conditioning Regimen 2: In some embodiments, a subject is administered a conditioning regimen comprising thiotepa, fludarabine, and TBI (e.g., HFTBI). In some embodiments, a subject is administered thiotepa at 5 mg/kg actual body weight or ideal body weight. In some embodiments, a subject is administered thiotepa at approximately 5 mg/kg for two days (e.g., consecutive days). In some embodiments, a subject is administered fludarabine at approximately 25 mg/m². (meters squared−body surface area). In some embodiments, a subject is administered fludarabine at approximately 25 mg/m² for three days (e.g., consecutive days). In some embodiments, a subject is administered HFTBI of 125 cGy (centigray). In some embodiments, a subject is administered HFTBI in 11 fractions of 125 cGy. In some embodiments, a subject is administered HFTBI in 11 fractions of 125 cGy over 4 days. In some embodiments, a subject is administered thiotepa at 5 mg/kg actual body weight or ideal body weight, is administered fludarabine at approximately 25 mg/m². (meters squared-body surface area), and is administered HFTBI of 125 cGy. In some embodiments, a subject is administered thiotepa at approximately 5 mg/kg for two days (e.g., consecutive days), is administered fludarabine at approximately 50 mg/m² for three days (e.g., consecutive days), and is administered HFTBI in 11 fractions of 125 cGy. In some embodiments, a subject is administered thiotepa at approximately 5 mg/kg on days −7 and −6, is administered fludarabine at approximately 50 mg/m² on days −5 to −3, and is administered HFTBI in 11 fractions of 125 cGy over 4 days.

A subject may receive the one or more cell populations described herein 1 day after completing a conditioning regimen. A subject may receive the one or more cell components described herein 2, 3, 4, 5, 6, 7, 8, 9, 10 days after completing a conditioning regimen. A subject may receive the one or more cell components described herein 1 day after receiving a final dose of a conditioning reagent such as TTP. A subject may receive the one or more cell components described herein 2, 3, 4, 5, 6, 7, 8, 9 or 10 days after receiving a final dose of a conditioning reagent such as TTP. A subject may receive the one or more cell components described herein 1 day after receiving a first dose of a conditioning reagent such as TTP. A subject may receive the one or more cell components described herein 2, 3, 4, 5, 6, 7, 8, 9 or 10 days after receiving a first dose of a conditioning reagent such as TTP.

### Graft Versus Host Disease (GVHD)

Graft versus host disease (GVHD) is a significant cause of morbidity and mortality in hematopoietic stem cell transplantation (HCT) recipients. Aspects and embodiments herein provide compositions and methods that reduce the incidence of GVHD, reduce the severity of GVHD, reduce the relative risk of GVHD, prevent GVHD, or a combination thereof in HCT recipients.

Graft-versus-host disease (GVHD) is an inflammatory disease that can occur in the allogeneic transplant setting. GVHD involves donor cells (graft) attacking recipient cells (host). GVHD can be life-threatening and can involve, for example, the skin, the intestines, and/or the liver. The morbidity and mortality associated GVHD can be a major factor limiting the success of HCT. GVHD can occur despite use of an HLA-matched sibling donor, and the use of various GVHD prophylactic/immunosuppressive agents, for example, use of two or more of tacrolimus, sirolimus, cyclosporine, methotrexate, mycophenolate, mycophenolate mofetil (MMF), anti-thymocyte globulin and corticosteroids.

In some embodiments, a multi-component pharmaceutical treatment or a multi-component cellular therapy product of the present disclosure further comprises from approximately 100 mg to approximately 5000 mg of mycophenolate mofetil (MMF). In some embodiments, a method of the present disclosure further comprises administering or otherwise providing from approximately 100 mg to approximately 5000 mg of mycophenolate mofetil (MMF). In some embodiments, the MMF is provided an amount or dose that is approximately 100 mg, approximately 150 mg, approximately 200 mg, approximately 250 mg, approximately 300 mg, approximately 350 mg, approximately 400 mg, approximately 450 mg, approximately 500 mg, approximately 550 mg, approximately 600 mg, approximately 650 mg, approximately 700 mg, approximately 750 mg, approximately 800 mg, approximately 850 mg, approximately 900 mg, approximately 950 mg, approximately 1000 mg, approximately 1050 mg, approximately 1100 mg, approximately 1150 mg, approximately 1200 mg, approximately 1250 mg, approximately 1300 mg, approximately 1350 mg, approximately 1400 mg, approximately 1450 mg, approximately 1500 mg, approximately 1550 mg, approximately 1600 mg, approximately 1650 mg, approximately 1700 mg, approximately 1750 mg, approximately 1800 mg, approximately 1850 mg, approximately 1900 mg, approximately 1950 mg, approximately 2000 mg, approximately 2050 mg, approximately 2100, approximately 2150 mg, approximately 2200 mg, approximately 2250 mg, approximately 2300 mg, approximately 2350 mg, approximately 2400 mg, approximately 2450 mg, approximately 2500 mg, approximately 2550 mg, approximately 2600 mg, approximately 2650 mg, approximately 2700 mg, approximately 2750 mg, approximately 2800 mg, approximately 2850 mg, approximately 2900 mg, approximately 2950 mg, approximately 3000 mg, approximately 3050 mg, approximately 3100, approximately 3150 mg, approximately 3200 mg, approximately 3250 mg, approximately 3300 mg, approximately 3350 mg, approximately 3400 mg, approximately 3450 mg, approximately 3500 mg, approximately 3550 mg, approximately 3600 mg, approximately 3650 mg, approximately 3700 mg, approximately 3750 mg, approximately 3800 mg, approximately 3850 mg, approximately 3900 mg, approximately 3950 mg, approximately 4000 mg, approximately 4050 mg, approximately 4100, approximately 4150 mg, approximately 4200 mg, approximately 4250 mg, approximately 4300 mg, approximately 4350 mg, approximately 4400 mg, approximately 4450 mg, approximately 4500 mg, approximately 4550 mg, approximately 4600 mg, approximately 4650 mg, approximately 4700 mg, approximately 4750 mg, approximately 4800 mg, approximately 4850 mg, approximately 4900 mg, approximately 4950 mg, or approximately 5000 mg.

### GVHD Classification and Grading

GVHD can be classified into acute GVHD (aGVHD) and chronic GVHD (cGVHD). In some embodiments, GVHD that occurs within the first 100 days post-transplant can be referred to as aGVHD, and GVHD after the first 100 days can be referred to as chronic GVHD (cGVHD). cGVHD is a major source of late treatment-related complications, and can be life-threatening. In addition to inflammation, cGVHD can lead to the development of fibrosis, which can result in functional disability.

Yet another aspect provides any herein-disclosed multi-component pharmaceutical treatment in which a risk and/or severity of an adverse event associated with the multi-component pharmaceutical treatment is reduced as compared to a similar pharmaceutical treatment in which a human subject receives Tcons but does not receive Tregs or is any herein-disclosed method in which a risk and/or severity of an adverse event associated with the method is reduced as compared to a similar method in which a human subject receives Tcons but does not receive Tregs.

In various embodiments, the adverse event is acute GVHD (aGVHD),

In some embodiments, the adverse event is stage two or greater aGVHD.

In embodiments, the adverse event is chronic GVHD (cGVHD).

In various embodiments, the human subject has no cGVHD about one year after being administered the cell populations.

In some embodiments, the adverse event is moderate to severe cGVHD.

In embodiments, the adverse event is acute graft vs host disease (aGVHD), e.g., stage two or greater aGVHD. In some cases, the patient has no stage two or higher aGVHD about 180 days after being administered the cell populations.

In various embodiments, the adverse event is chronic graft vs host disease (cGVHD). In some cases, the patient has no cGVHD about one year after being administered the cell populations.

In some embodiments, the adverse event is moderate to severe cGVHD. In some cases, the patient does not have moderate to severe cGVHD about one year after being administered the cell populations.

In embodiments, a patient does not develop GVHD within about 30 days of administration of the Tcons, does not develop GVHD within about 100 days of administration of the Tcons, does not develop GVHD within about 180 days of administration of the Tcons, and/or does not develop GVHD within about one year of administration of the Tcons.

In various embodiments, a human subject does not develop higher than stage 2 GVHD within about 100 days of the administering of the third population of CD45⁺ cells, the human subject does not develop higher than stage 2 GVHD) within about 180 days or within about 200 days of the administering of the third population of CD45⁺ cells, the human subject does not develop higher than stage 2 GVHD within about 1 year of the administering of the third population of CD45⁺ cells.

aGVHD and cGVHD can be graded using a system that first evaluates GVHD stages for the skin, liver, and gut, and then combines scoring from the organ staging to determine an overall GVHD grade. An example of a GVHD staging criteria that can be used for individual organs are provided in TABLE 1:

TABLE 1

| Organ | Stage | Description |
|---|---|---|
| Skin | 0 | No active (erythematous) GVHD rash |
| | 1 | Maculo-papular eruption over <25% of body area |
| | 2 | Maculo-papular eruption over >25 to 50% of body area |
| | 3 | Generalized erythroderma/maculo-papular eruption |
| | | over >50% of body area |
| | 4 | Generalized erythroderma/maculo-papular eruption |
| | | over >50% of body area plus bullous formation and |
| | | desquamation over >5% of body area |
| Liver | 0 | Bilirubin <2 mg/dL |
| | 1 | Bilirubin 2.0 to 3.0 mg/dL; |
| | | serum glutamic oxaloacetic transaminase (SGOT) SGOT |
| | | 150 to 750 IU |
| | 2 | Bilirubin 3.1 to 6.0 mg/dL |
| | 3 | Bilirubin 6.1 to 15.0 mg/dL |
| | 4 | Bilirubin >15.0 mg/dL |
| Gut | 0 | <500 mL/day or <3 episodes/day |
| | 1 | Diarrhea >30 mL/kg or >500 mL/day |
| | 2 | Diarrhea >60 mL/kg or >1000 mL/day |
| | 3 | Diarrhea >90 mL/kg or >1500 mL/day |
| | 4 | Diarrhea >90 mL/kg or >2000 mL/day; or severe |
| | | abdominal pain with or without ileus |

TABLE 2

| | | | | | ECOG |
|---|---|---|---|---|---|
| Grade | Skin | Liver | | Gut | performance |
| 1 | 1 to 2 | 0 | | 0 | 0 |
| 2 | 1 to 3 | 1 | and/or | 1 | 0-1 |
| 3 | 2 to 3 | 2 to 4 | and/or | 2 to 3 | 2-3 |
| 4 | 2 to 4 | 2 to 4 | and/or | 2 to 3 | 3-4 |

aGVHD grade can also be determined based on most severe target organ involvement as defined in the MAGIC standardization criteria described by Harris et al., “International, multicenter standardization of acute graft-versus-host disease clinical data collection: a report from the Mount Sinai Acute GVHD International Consortium.” Biology of Blood and Marrow Transplantation 22.1 (2016): 4-10. For example, aGVHD organ staging can be evaluated according to TABLE 3, and overall aGVHD grade can be assessed as follows:
Grade 0: No Stage 1-4 of any organ.
Grade I: Stage 1-2 skin without liver, upper GI, or lower GI involvement.
Grade II: Stage 3 rash and/or Stage 1 liver and/or Stage 1 upper GI and/or Stage 1 lower GI.
Grade III: Stage 2-3 liver and/or Stage 2-3 lower GI, with Stage 0-3 skin and/or Stage 0-1 upper GI. Grade IV: Stage 4 skin, liver, or lower GI involvement, with Stage 0-1 upper GI.

TABLE 3

| | Skin (Active | Liver | | | Lower GI |
|---|---|---|---|---|---|
| Stage | Erythema Only) | (Bilirubin) | | Upper GI | (stool output/day) |
| 0 | No active | <2 | mg/dL | No or intermittent | Adult: <500 mL/day |
| | (erythematous) | | | nausea, vomiting | or <3 episodes/day |
| | GvHD rash | | | or anorexia | Child: <10 mL/kg/day |
| | | | | | or <4 episodes/day |
| 1 | Maculopapular | 2-3 | mg/dL | Persistent nausea, | Adult: 500-999 mL/day |
| | rash <25% BSA | | | vomiting or | or 3-4 episodes/day |
| | | | | anorexia | Child: 10-19.9 mL/kg/day |
| | | | | | or 5-7 episodes/day |
| 2 | Maculopapular | 3.1-6 | mg/dL | | Adult: 1000-1500 mL/day |
| | rash 25-50% BSA | | | | or 57 episodes/day |
| | | | | | Child: 20-30 mL/kg/day |
| | | | | | or 7-10 episodes/day |
| 3 | Maculopapular | 6.1-15 | mg/dL | | Adult: >1500 mL/day |
| | rash >50% BSA | | | | or >7 episodes/day |
| | | | | | Child: >30 mL/kg/day |
| | | | | | or >10 episodes/day |
| 4 | Generalized | >15 | mg/dL | | Severe abdominal pain |
| | erythroderma | | | | with or without ileus |
| | (>50% BSA) | | | | or grossly bloody stool |
| | plus bullous | | | | (regardless of stool |
| | formation and | | | | volume) |
| | desquamation | | | | |
| | >5% BSA | | | | |

In some embodiments, GVHD stage and GVHD grade are synonyms.

cGVHD can also be assessed by the method described by Jagasia et al., “National Institutes of Health consensus development project on criteria for clinical trials in chronic graft-versus-host disease: I. The 2014 diagnosis and staging working group report.” Biology of Blood and Marrow Transplantation 21.3 (2015): 389-401. To establish a diagnosis of cGVHD, these criteria can require, for example, at least one diagnostic manifestation of chronic GVHD or at least one distinctive manifestation plus a pertinent biopsy, laboratory, or other test (e.g., PFTs, Schirmer's test), evaluation by a specialist (ophthalmologist, gynecologist) or radiographic imaging showing chronic GVHD in the same or another organ.

Organ systems can be scored as described in Jagasia et al., and Mild cGVHD can be present when one or two organs are involved with no more than score 1, plus a lung score of zero; moderate cGVHD can be present when three or more organs are involved with no more than score 1, or when at least one non-lung organ has a score of 2, or when the lungs have a score of 1; and severe cGVHD can be present when at least one organ has a score of 4, or the lungs have a score of 2 or 3.

TABLE 4 provides diagnostic and distinctive manifestations of cGVHD. infection, drug effect, malignancy, or other causes are excluded for distinctive manifestations. Bronchiolitis obliterans syndrome can be diagnostic for lung chronic GVHD only if distinctive sign or symptom present in another organ. Diagnosis of chronic GVHD based on myositis or polymyositis can require a biopsy.

TABLE 4

| | Diagnostic (Sufficient to | Distinctive¹ (Seen in chronic GVHD, |
|---|---|---|
| Organ or | Establish the Diagnosis | but Insufficient Alone |
| Site | of chronic GVHD) | to Establish a Diagnosis) |
| Skin | Poikiloderma Lichen | Depigmentation Papulosquamous |
| | planus-like features | lesions |
| | Sclerotic features | |
| | Morphea-like features | |
| | Lichen | |
| | sclerosus-like features | |
| Nails | | Dystrophy |
| | | Longitudinal ridging, splitting or brittle features |
| | | Onycholysis |
| | | Pterygium unguis |
| | | Nail loss (usually symmetric, affects most nails) |
| Scalp and | | New onset of scarring or nonscarring scalp |
| body hair | | alopecia (after recovery from |
| | | chemoradiotherapy) |
| | | Loss of body hair |
| Mouth | Lichen planus-like changes | Xerostomia Mucoceles Mucosal |
| | | atrophy Ulcers |
| | | Pseudomembranes |
| Eyes | | New onset dry, gritty, or painful eyes |
| | | Cicatricial conjunctivitis |
| | | Keratoconjunctivitis sicca |
| | | Confluent areas of punctate keratopathy |
| Genitalia | Lichen planus-like features | Erosions Fissures |
| Females | Lichen sclerosus-like features | |
| Males | Vaginal scarring or clitoral/ | Ulcers |
| | labial agglutination | |
| | Phimosis or urethral/meatus | |
| | scarring or stenosis | |
| GI Tract | Esophageal web Strictures or | |
| | stenosis in the upper to | |
| | mid third of the esophagus | |
| Lung | Bronchiolitis obliterans | Air trapping and bronchiectasis |
| | diagnosed with lung biopsy | on chest CT |
| | Bronchiolitis obliterans | |
| | syndrome² | |
| Muscles, | Joint stiffness or contractures | Myositis or polymyositis³ |
| fascia, joints | secondary to fasciitis or | |
| | sclerosis | |

In some embodiments, GVHD severity can be graded using the Glucksberg grade (I-IV) or the International Bone Marrow Transplant Registry (IBMTR) grading system (A-D). The severity of acute GVHD can be determined by an assessment of the degree of involvement of the skin, liver, and gastrointestinal tract. The stages of individual organ involvement are combined with (Glucksberg) or without (IBMTR) the patient's performance status to produce an overall grade.

### GVHD Prophylaxis and Treatment

Immunosuppressive agents can be used to reduce the likelihood of GVHD (GVHD prophylactic agents), or to treat GVHD once it occurs (GVHD therapeutic agents). However, in some cases, the use of GVHD prophylactic agents, GVHD therapeutic agents, or both can be insufficient to effectively prevent or treat GVHD. For example, the incidence of GVHD in graft recipients can be high despite use of use of tacrolimus, sirolimus, cyclosporine, methotrexate, mycophenolate, anti-thymocyte globulin, corticosteroids, or a combination thereof (e.g., two or more of the agents).

Administering multiple GVHD prophylactic and/or therapeutic agents, high doses of GVHD prophylactic and/or therapeutic agents, or both can fail to effectively treat GVHD in many alloHSCT settings or can result in increased susceptibility to infection and decreased graft versus tumor therapeutic effects.

Non-limiting examples of GVHD prophylactic and/or GVHD therapeutic agents that can be used include calcineurin inhibitors (e.g., tacrolimus, cyclosporine A), sirolimus, monoclonal antibodies, methotrexate, mycophenolate, anti-thymocyte globulin, corticosteroids, azathioprine, and mycophenolate mofetil. Monoclonal antibodies useful as immunosuppressive agents include, for example, antagonist antibodies, (e.g., antibodies that antagonize IL-2R such as basiliximab and daclizumab), and antibodies that deplete an immune cell population by antibody dependent cellular cytotoxicity (e.g., anti-CD3 antibodies for T cell depletion such as muromonab-CD3).

Compositions and methods described herein may comprise administering one or more GVHD prophylactic agents to an HCT recipient. GVHD prophylaxis in such cases should be considered different from GVHD treatment such that the GVHD prophylactic agent will be administered to the HCT recipient before an incidence of GVHD is assessed. In some cases, an HCT recipient may be administered one or more GVHD prophylactic agents but not a GVHD therapeutic agent. In some cases, a HCT recipient does not require treatment for GVHD and/or does not receive treatment for GVHD.

Compositions and methods disclosed herein can reduce the incidence of GVHD, reduce the severity of GVHD, reduce the relative risk of GVHD, prevent GVHD, or a combination thereof in HCT recipients. In some embodiments, such benefits are achieved despite administering no GVHD prophylactic agents as disclosed herein. In some embodiments, such benefits are achieved despite administering a reduced number of GVHD prophylactic agents (e.g., a single GVHD prophylactic agents), a low dose of GVHD prophylactic agent(s), or a combination thereof as disclosed herein. In some embodiments, 1 GVHD prophylactic agent is administered to a subject. In some embodiments, no more than GVHD prophylactic agent is administered to a subject. In some embodiments, 2 GVHD prophylactic agents are administered to a subject. In some embodiments, no more than 2 GVHD prophylactic agents are administered to a subject.

In some embodiments, one or more GVHD prophylactic agents may be administered to a HCT recipient for a duration of 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 18 months, 24 months, 36 months post-transplant of one or more cell populations. One or more GVHD prophylactic agents may be administered to an HCT recipient starting the day of transplant of one or more cell populations. For instance, a GVHD prophylactic regimen may begin the day an HSPC cell population and/or a Treg cell population is administered to the recipient. Alternatively, a GVHD prophylactic regimen may begin the day a Tcon cell population is administered to the patient.

In some embodiments, tacrolimus is not administered to a subject. In some embodiments, sirolimus is not administered to a subject. In some embodiments, cyclosporine is not administered to a subject. In some embodiments, methotrexate is not administered to a subject. In some embodiments, mycophenolate is not administered to a subject. In some embodiments, anti-thymocyte globulin is not administered to a subject. In some embodiments, corticosteroids are not administered to a subject.

In some embodiments, the GVHD prophylactic agent is tacrolimus.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from approximately 12 to approximately 24 hours after administration of the T-cons. In some cases, the tacrolimus GHVDPA is administered for a period of time up to approximately 90 days, is administered for a period of time up to approximately 60 days. In some embodiments, the tacrolimus GHVDPA is initially administered to the patient at approximately 0.03 mg/kg patient's actual or ideal body weight/day. In some cases, a dose of the tacrolimus GVHDPA administered to the patient is tapered starting at approximately 90 days after a first dose is administered to the patient or is tapered starting at approximately 45 days after a first dose is administered to the patient.

Aspects and embodiments herein provide a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In cases where aGVHD occurs, responsiveness of aGVHD to GVHD therapeutic agents (e.g., corticosteroids) can be assessed by the criteria of TABLE 5.

TABLE 5

| | |
|---|---|
| Complete | Complete resolution of acute GvHD symptoms |
| response (CR) | in all organs, without secondary GvHD therapy |
| Partial | Improvement in GvHD stage in all initial GvHD |
| response (PR) | target organs without complete resolution and |
| | without worsening in any other GvHD target |
| | organs, without secondary GvHD therapy |
| Very good partial | Improvement in GvHD in all initial GvHD target |
| response (VGPR) | organs, with maximum Stage I involvement in |
| | one or more organs (except upper gastrointestinal |
| | tract), without secondary GvHD therapy |
| No response | Same grade of GvHD or progression of GvHD in |
| (NR) | any organ or death, or the addition of secondary |
| | GvHD therapy |
| Progression | Worsening GvHD in at least 1 organ with or |
| | without amelioration in any organ |

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a very good partial response to GVHD therapeutic agents.

In cases where cGVHD occurs, responsiveness of cGVHD to GVHD therapeutic agents (e.g., corticosteroids) can be assessed by the criteria of TABLE 6. Abbreviations used: ALT, alanine transaminase; FEV1, forced expiratory volume in the first second; OMRS, Oral Mucosa Rating Scale; PFTs, pulmonary function tests; P-ROM, photographic range of motion; ULN, upper limit of normal.

TABLE 6

| Organ | Complete Response | Partial Response | Progression |
|---|---|---|---|
| Skin | NIH Skin Score 0 after | Decrease in NIH Skin | Increase in NIH Skin Score |
| | previous involvement | Score by 1 or more points | by 1 or more points, except |
| | | | 0 to 1 |
| Eyes | NIH Eye Score 0 after | Decrease in NIH Eye Score | Increase in NIH Eye Score |
| | previous involvement | by 1 or more points | by 1 or more points, except |
| | | | 0 to 1 |
| Mouth | NIH Modified OMRS 0 | Decrease in NIH Modified | Increase in NIH Modified |
| | after previous involvement | OMRS of 2 or more points | OMRS of 2 or more points |
| Esophagus | NIH Esophagus Score 0 after | Decrease in NIH | Increase in NIH Esophagus |
| | previous involvement | Esophagus Score by 1 or | Score by 1 or more points, |
| | | more points | except 0 to 1 |
| Upper GI | NIH Upper GI Score 0 after | Decrease in NIH Upper GI | Increase in NIH Upper GI |
| | previous involvement | Score by 1 or more points | Score by 1 or more points, |
| | | | except 0 to 1 |
| Lower GI | NIH Lower GI Score 0 after | Decrease in NIH Lower GI | Increase in NIH Lower GI |
| | previous involvement | Score by 1 or more points | Score by 1 or more points, |
| | | | except from 0 to 1 |
| Liver | Normal ALT, alkaline | Decrease by 50% | Increase by 2 × ULN |
| | phosphatase, and Total | | |
| | bilirubin after previous | | |
| | elevation of 1 or more | | |
| Lungs | Normal % FEV1 after | Increase by 10% predicted | Decrease by 10% predicted |
| | previous involvement | absolute value of % FEV1 | absolute value of % FEV1 |
| | If PFTs not available, | If PFTs not available, | If PFTs not available, |
| | NIH Lung Symptom Score 0 | decrease in NIH Lung | increase in NIH Lung - |
| | after previous involvement | Symptom Score by 1 or | Symptom Score by 1 or |
| | | more points | more points, except 0 to |
| | | | 1 |
| Joints and | Both NIH Joint and Fascia | Decrease in NIH Joint and | Increase in NIH Joint and |
| fascia | Score 0 and P-ROM score 25 | Fascia Score by 1 or more | Fascia Score by 1 or more |
| | after previous involvement | points or increase in P-ROM | points or decrease in P-ROM |
| | by at least 1 measure | score by 1 point for any site | score by 1 point for any site |
| Global | Clinician overall severity | Clinician overall severity | Clinician overall severity |
| | score 0 | score decreases by 2 or more | score increases by 2 or more |
| | | points on a 0-10 scale | points on a 0-10 scale |

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

### Acute GVHD Incidence

Subjects administered a composition of the disclosure (e.g., a cell population as described herein) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 aGVHD, for example, within 30 days post-transplant, 100 days post-transplant, or within another suitable amount of time post-transplant as disclosed herein.

As used herein, an alternate composition lacks one or more cell populations and/or prophylactic agent that are disclosed herein and/or recited in the claims. As examples, an alternate composition lacks one or more of a first population of CD45⁺ cells that comprises, at least, HSPCs, a cell population enriched for Tregs, a third population of CD45⁺ cells that comprises, at least, Tcons, and a prophylactic agent.

Subjects administered a composition of the disclosure (e.g., a cell population as described herein) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 2 aGVHD. In some embodiments, less than about 15% of subjects that are administered a composition of the disclosure develop ≥grade 2 aGVHD. In some embodiments, less than about 10% of subjects that are administered a composition of the disclosure develop ≥grade 2 aGVHD. In some embodiments, less than about 8% of subjects that are administered a composition of the disclosure develop ≥grade 2 aGVHD. In some embodiments, less than about 7% of subjects that are administered a composition of the disclosure develop ≥grade 2 aGVHD.

Subjects administered a composition of the disclosure (e.g., a cell population as described herein) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 3 aGVHD. In some embodiments, less than about 15% of subjects that are administered a composition of the disclosure develop ≥grade 3 aGVHD. In some embodiments, less than about 10% of subjects that are administered a composition of the disclosure develop ≥grade 3 aGVHD. In some embodiments, less than about 5% of subjects that are administered a composition of the disclosure develop ≥grade 3 aGVHD. In some embodiments, less than about 3% of subjects that are administered a composition of the disclosure develop ≥grade 3 aGVHD. In some embodiments, less than about 2% of subjects that are administered a composition of the disclosure develop ≥grade 3 aGVHD. In some embodiments, less than about 1% of subjects that are administered a composition of the disclosure develop ≥grade 3 aGVHD.

Subjects administered a composition of the disclosure (e.g., a cell population as described herein) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure develop ≥grade 4 aGVHD. In some embodiments, less than about 5% of subjects that are administered a composition of the disclosure develop ≥grade 4 aGVHD. In some embodiments, less than about 3% of subjects that are administered a composition of the disclosure develop ≥grade 4 aGVHD. In some embodiments, less than about 2% of subjects that are administered a composition of the disclosure develop ≥grade 4 aGVHD. In some embodiments, less than about 1% of subjects that are administered a composition of the disclosure develop ≥grade 4 aGVHD.

The incidence of aGVHD can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days post-transplant.

The incidence of aGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, subjects administered a composition of the disclosure (e.g., a cell population as described herein) have a decreased wherein incidence, severity, timing, or any combination thereof of Grade I to Grade IV acute GVHD (aGVHD), relative to a corresponding human subject that has been administered a standard myeloablative allogeneic hematopoietic stem cell transplant (alloHSCT). In some embodiments, the subject is free of Grade I to Grade IV aGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have increased aGVHD survival, relative to a corresponding human subject that has been administered a standard myeloablative allogeneic hematopoietic stem cell transplant (alloHSCT). In some embodiments, the subject experiences aGHVD-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have decreased incidence of steroid-refractory aGVHD, relative to a corresponding human subject that has been administered a standard myeloablative allogeneic hematopoietic stem cell transplant (alloHSCT). In some embodiments, the subject is free of steroid-refractory aGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

### No GVHD Prophylaxis

Subjects administered a composition of the disclosure (e.g., a cell population as described herein) in the absence of GVHD prophylactic agents exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) in the absence of GVHD prophylactic agents exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 aGVHD, for example, within 30 days post-transplant, 100 days post-transplant, or within another suitable amount of time post-transplant as disclosed herein.

In some embodiments, less than about 50% of subjects that are administered a composition of the disclosure in the absence of GVHD prophylactic agents develop ≥grade 2 aGVHD. In some embodiments, less than about 40% of subjects that are administered a composition of the disclosure in the absence of GVHD prophylactic agents develop ≥grade 2 aGVHD. In some embodiments, less than about 30% of subjects that are administered a composition of the disclosure in the absence of GVHD prophylactic agents develop ≥grade 2 aGVHD. In some embodiments, less than about 25% of subjects that are administered a composition of the disclosure in the absence of GVHD prophylactic agents develop ≥grade 2 aGVHD. In some embodiments, less than about 20% of subjects that are administered a composition of the disclosure in the absence of GVHD prophylactic agents develop ≥grade 2 aGVHD. In some embodiments, less than about 10% of subjects that are administered a composition of the disclosure in the absence of GVHD prophylactic agents develop ≥grade 2 aGVHD.

Subjects administered a composition of the disclosure (e.g., a cell population as described herein) in the absence of GVHD prophylactic agents exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 aGVHD, for example, within 30 days post-transplant, 100 days post-transplant, or within another suitable amount of time post-transplant as disclosed herein.

In some embodiments, less than about 40% of subjects that are administered a composition of the disclosure in the absence of GVHD prophylactic agents develop ≥grade 3 aGVHD. In some embodiments, less than about 30% of subjects that are administered a composition of the disclosure in the absence of GVHD prophylactic agents develop ≥grade 3 aGVHD. In some embodiments, less than about 20% of subjects that are administered a composition of the disclosure in the absence of GVHD prophylactic agents develop ≥grade 3 aGVHD. In some embodiments, less than about 10% of subjects that are administered a composition of the disclosure in the absence of GVHD prophylactic agents develop ≥grade 3 aGVHD. In some embodiments, less than about 5% of subjects that are administered a composition of the disclosure in the absence of GVHD prophylactic agents develop ≥grade 3 aGVHD. In some embodiments, less than about 3% of subjects that are administered a composition of the disclosure in the absence of GVHD prophylactic agents develop ≥grade 3 aGVHD. In some embodiments, less than about 2% of subjects that are administered a composition of the disclosure in the absence of GVHD prophylactic agents develop ≥grade 3 aGVHD. In some embodiments, less than about 1% of subjects that are administered a composition of the disclosure in the absence of GVHD prophylactic agents develop ≥grade 3 aGVHD.

Subjects administered a composition of the disclosure (e.g., a cell population as described herein) in the absence of GVHD prophylactic agents exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure in the absence of GVHD prophylactic agents develop ≥grade 4 aGVHD, for example, within 30 days post-transplant, 100 days post-transplant, or within another suitable amount of time post-transplant as disclosed herein.

In some embodiments, less than about 20% of subjects that are administered a composition of the disclosure in the absence of GVHD prophylactic agents develop ≥grade 4 aGVHD. In some embodiments, less than about 15% of subjects that are administered a composition of the disclosure in the absence of GVHD prophylactic agents develop ≥grade 4 aGVHD. In some embodiments, less than about 10% of subjects that are administered a composition of the disclosure in the absence of GVHD prophylactic agents develop ≥grade 4 aGVHD. In some embodiments, less than about 5% of subjects that are administered a composition of the disclosure in the absence of GVHD prophylactic agents develop ≥grade 4 aGVHD. In some embodiments, less than about 3% of subjects that are administered a composition of the disclosure in the absence of GVHD prophylactic agents develop ≥grade 4 aGVHD. In some embodiments, less than about 2% of subjects that are administered a composition of the disclosure in the absence of GVHD prophylactic agents develop ≥grade 4 aGVHD. In some embodiments, less than about 1% of subjects that are administered a composition of the disclosure in the absence of GVHD prophylactic agents develop ≥grade 4 aGVHD.

The absence of GVHD prophylactic agents can refer to cases where no GVHD prophylactic agents are administered to the subject for the first 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 105 days, 110 days, 112 days, 115 days, 119 days, or 120 days post-transplant.

### Single Agent GVHD Prophylaxis

Subjects administered a composition of the disclosure (e.g., a cell population as described herein) and no more than one GVHD prophylactic agent (for example, a single GVHD prophylactic agent) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 2 aGVHD. In some embodiments, less than about 15% of subjects that are administered a composition of the disclosure and no more than one GVHD prophylactic agent (for example, a single GVHD prophylactic agent) develop ≥grade 2 aGVHD. In some embodiments, less than about 10% of subjects that are administered a composition of the disclosure and no more than one GVHD prophylactic agent (for example, a single GVHD prophylactic agent) develop ≥grade 2 aGVHD. In some embodiments, less than about 8% of subjects that are administered a composition of the disclosure and no more than one GVHD prophylactic agent (for example, a single GVHD prophylactic agent) develop ≥grade 2 aGVHD. In some embodiments, less than about 7% of subjects that are administered a composition of the disclosure and no more than one GVHD prophylactic agent (for example, a single GVHD prophylactic agent) develop ≥grade 2 aGVHD.

Subjects administered a composition of the disclosure (e.g., a cell population as described herein) and no more than one GVHD prophylactic agent (for example, a single GVHD prophylactic agent) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 3 aGVHD. In some embodiments, less than about 15% of subjects that are administered a composition of the disclosure and no more than one GVHD prophylactic agent (for example, a single GVHD prophylactic agent) develop ≥grade 3 aGVHD. In some embodiments, less than about 10% of subjects that are administered a composition of the disclosure and no more than one GVHD prophylactic agent (for example, a single GVHD prophylactic agent) develop ≥grade 3 aGVHD. In some embodiments, less than about 5% of subjects that are administered a composition of the disclosure and no more than one GVHD prophylactic agent (for example, a single GVHD prophylactic agent) develop ≥grade 3 aGVHD. In some embodiments, less than about 3% of subjects that are administered a composition of the disclosure and no more than one GVHD prophylactic agent (for example, a single GVHD prophylactic agent) develop ≥grade 3 aGVHD. In some embodiments, less than about 2% of subjects that are administered a composition of the disclosure and no more than one GVHD prophylactic agent (for example, a single GVHD prophylactic agent) develop ≥grade 3 aGVHD. In some embodiments, less than about 1% of subjects that are administered a composition of the disclosure and no more than one GVHD prophylactic agent (for example, a single GVHD prophylactic agent) develop ≥grade 3 aGVHD.

Subjects administered a composition of the disclosure (e.g., a cell population as described herein) and no more than one GVHD prophylactic agent (for example, a single GVHD prophylactic agent) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and no more than one GVHD prophylactic agent (for example, a single GVHD prophylactic agent) develop ≥grade 4 aGVHD. In some embodiments, less than about 5% of subjects that are administered a composition of the disclosure and no more than one GVHD prophylactic agent (for example, a single GVHD prophylactic agent) develop ≥grade 4 aGVHD. In some embodiments, less than about 3% of subjects that are administered a composition of the disclosure and no more than one GVHD prophylactic agent (for example, a single GVHD prophylactic agent) develop ≥grade 4 aGVHD. In some embodiments, less than about 2% of subjects that are administered a composition of the disclosure and no more than one GVHD prophylactic agent (for example, a single GVHD prophylactic agent) develop ≥grade 4 aGVHD. In some embodiments, less than about 1% of subjects that are administered a composition of the disclosure and no more than one GVHD prophylactic agent (for example, a single GVHD prophylactic agent) develop ≥grade 4 aGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 2 aGVHD. In some embodiments, less than about 15% of subjects that are administered a composition of the disclosure and a low dose of a GVHD prophylactic agent (for example, a single GVHD prophylactic agent at a low dose) develop ≥grade 2 aGVHD. In some embodiments, less than about 10% of subjects that are administered a composition of the disclosure and a low dose of a GVHD prophylactic agent (for example, a single GVHD prophylactic agent at a low dose) develop ≥grade 2 aGVHD. In some embodiments, less than about 8% of subjects that are administered a composition of the disclosure and a low dose of a GVHD prophylactic agent (for example, a single GVHD prophylactic agent at a low dose) develop ≥grade 2 aGVHD. In some embodiments, less than about 7% of subjects that are administered a composition of the disclosure and a low dose of a GVHD prophylactic agent (for example, a single GVHD prophylactic agent at a low dose) develop ≥grade 2 aGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 3 aGVHD. In some embodiments, less than about 15% of subjects that are administered a composition of the disclosure and a low dose of a GVHD prophylactic agent (for example, a single GVHD prophylactic agent at a low dose) develop ≥grade 3 aGVHD. In some embodiments, less than about 10% of subjects that are administered a composition of the disclosure and a low dose of a GVHD prophylactic agent (for example, a single GVHD prophylactic agent at a low dose) develop ≥grade 3 aGVHD. In some embodiments, less than about 5% of subjects that are administered a composition of the disclosure and a low dose of a GVHD prophylactic agent (for example, a single GVHD prophylactic agent at a low dose) develop ≥grade 3 aGVHD. In some embodiments, less than about 3% of subjects that are administered a composition of the disclosure and a low dose of a GVHD prophylactic agent (for example, a single GVHD prophylactic agent at a low dose) develop ≥grade 3 aGVHD. In some embodiments, less than about 2% of subjects that are administered a composition of the disclosure and a low dose of a GVHD prophylactic agent (for example, a single GVHD prophylactic agent at a low dose) develop ≥grade 3 aGVHD. In some embodiments, less than about 1% of subjects that are administered a composition of the disclosure and a low dose of a GVHD prophylactic agent (for example, a single GVHD prophylactic agent at a low dose) develop ≥grade 3 aGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and a low dose of a GVHD prophylactic agent (for example, a single GVHD prophylactic agent at a low dose) develop ≥grade 4 aGVHD. In some embodiments, less than about 5% of subjects that are administered a composition of the disclosure and a low dose of a GVHD prophylactic agent (for example, a single GVHD prophylactic agent at a low dose) develop ≥grade 4 aGVHD. In some embodiments, less than about 3% of subjects that are administered a composition of the disclosure and a low dose of a GVHD prophylactic agent (for example, a single GVHD prophylactic agent at a low dose) develop ≥grade 4 aGVHD. In some embodiments, less than about 2% of subjects that are administered a composition of the disclosure and a low dose of a GVHD prophylactic agent (for example, a single GVHD prophylactic agent at a low dose) develop ≥grade 4 aGVHD. In some embodiments, less than about 1% of subjects that are administered a composition of the disclosure and a low dose of a GVHD prophylactic agent (for example, a single GVHD prophylactic agent at a low dose) develop ≥grade 4 aGVHD.

A low dose of a GVHD prophylactic agent can be, for example a target trough level of less than about 25 ng/mL, less than about 20 ng/ml, less than about 15 ng/mL, less than about 12 ng/mL, less than about 11 ng/ml, less than about 10 ng/ml, less than about 9 ng/ml, less than about 8 ng/mL, less than about 7 ng/ml, less than about 6 ng/mL, less than about 5 ng/ml, less than about 4 ng/mL, less than about 3 ng/mL, less than about 2 ng/ml, or less than about 1 ng/mL.

In some embodiments, a low dose of a GVHD prophylactic is a target trough level of approximately 1-25 ng/mL, about 1-20 ng/ml, about 1-15 ng/ml, about 1-12 ng/ml, about 1-11 ng/mL, about 1-10 ng/ml, about 1-9 ng/mL, about 1-8 ng/ml, about 1-7 ng/ml, about 1-6 ng/mL, about 1-5 ng/mL, about 1-4 ng/ml, about 1-3 ng/ml, about 1-2 ng/ml, about 2-25 ng/ml, about 2-20 ng/mL, about 2-15 ng/mL, about 2-12 ng/ml, about 2-11 ng/ml, about 2-10 ng/ml, about 2-9 ng/mL, about 2-8 ng/mL, about 2-7 ng/mL, about 2-6 ng/mL, about 2-5 ng/ml, about 2-4 ng/mL, about 2-3 ng/mL, about 3-25 ng/ml, about 3-20 ng/ml, about 3-15 ng/ml, about 3-12 ng/mL, about 3-11 ng/ml, about 3-10 ng/mL, about 3-9 ng/ml, about 3-8 ng/ml, about 3-7 ng/mL, about 3-6 ng/ml, about 3-5 ng/ml, about 3-4 ng/ml, about 4-25 ng/ml, about 4-20 ng/mL, about 4-15 ng/ml, about 4-12 ng/ml, about 4-11 ng/ml, about 4-10 ng/ml, about 4-9 ng/ml, about 4-8 ng/mL, about 4-7 ng/ml, about 4-6 ng/mL, about 4-5 ng/mL, about 5-25 ng/ml, about 5-20 ng/mL, about 5-15 ng/ml, about 5-12 ng/ml, about 5-11 ng/ml, about 5-10 ng/ml, about 5-9 ng/mL, about 5-8 ng/mL, about 5-7 ng/mL, about 5-6 ng/ml, about 6-25 g/mL, about 6-20 ng/mL, about 6-15 ng/ml, about 6-12 ng/mL, about 6-11 ng/ml, about 6-10 ng/ml, about 6-9 ng/mL, about 6-8 ng/mL, about 6-7 ng/ml, about 8-25 ng/ml, about 8-20 ng/mL, about 8-15 ng/mL, about 8-12 ng/mL, about 8-11 ng/mL, about 8-10 ng/ml, about 8-9 ng/ml, about 10-25 ng/mL, about 10-20 ng/ml, about 10-15 ng/ml, about 10-12 ng/mL, or about 10-11 ng/ml.

In some embodiments, a low dose of a GVHD prophylactic agent is tacrolimus with a target trough level of approximately 5 ng/mL to approximately 10 ng/mL. In some embodiments, a low dose of a GVHD prophylactic agent is tacrolimus with a target trough level of approximately 4 ng/ml to approximately 6 ng/mL.

In some embodiments, a low dose of a GVHD prophylactic agent is sirolimus with a target trough level of approximately 3 ng/mL to approximately 8 ng/mL. In some embodiments, a low dose of a GVHD prophylactic agent is sirolimus with a target trough level of approximately 4 ng/ml to approximately 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Chronic GVHD Incidence

Subjects administered a composition of the disclosure (e.g., a cell population as described herein) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 2 cGVHD. In some embodiments, less than about 40% of subjects that are administered a composition of the disclosure develop ≥grade 2 cGVHD. In some embodiments, less than about 30% of subjects that are administered a composition of the disclosure develop ≥grade 2 cGVHD. In some embodiments, less than about 20% of subjects that are administered a composition of the disclosure develop ≥grade 2 cGVHD. In some embodiments, less than about 10% of subjects that are administered a composition of the disclosure develop ≥grade 2 cGVHD. In some embodiments, less than about 5% of subjects that are administered a composition of the disclosure develop ≥grade 2 cGVHD.

Subjects administered a composition of the disclosure (e.g., a cell population as described herein) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 3 cGVHD. In some embodiments, less than about 40% of subjects that are administered a composition of the disclosure develop ≥grade 3 cGVHD. In some embodiments, less than about 30% of subjects that are administered a composition of the disclosure develop ≥grade 3 cGVHD. In some embodiments, less than about 20% of subjects that are administered a composition of the disclosure develop ≥grade 3 cGVHD. In some embodiments, less than about 15% of subjects that are administered a composition of the disclosure develop ≥grade 3 cGVHD. In some embodiments, less than about 10% of subjects that are administered a composition of the disclosure develop ≥grade 3 cGVHD. In some embodiments, less than about 5% of subjects that are administered a composition of the disclosure develop ≥grade 3 cGVHD. In some embodiments, less than about 3% of subjects that are administered a composition of the disclosure develop ≥grade 3 cGVHD. In some embodiments, less than about 2% of subjects that are administered a composition of the disclosure develop ≥grade 3 cGVHD. In some embodiments, less than about 1% of subjects that are administered a composition of the disclosure develop ≥grade 3 cGVHD.

Subjects administered a composition of the disclosure (e.g., a cell population as described herein) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure develop ≥grade 4 cGVHD. In some embodiments, less than about 30% of subjects that are administered a composition of the disclosure develop ≥grade 4 cGVHD. In some embodiments, less than about 20% of subjects that are administered a composition of the disclosure develop ≥grade 4 cGVHD. In some embodiments, less than about 10% of subjects that are administered a composition of the disclosure develop ≥grade 4 cGVHD. In some embodiments, less than about 5% of subjects that are administered a composition of the disclosure develop ≥grade 4 cGVHD. In some embodiments, less than about 3% of subjects that are administered a composition of the disclosure develop ≥grade 4 cGVHD. In some embodiments, less than about 2% of subjects that are administered a composition of the disclosure develop grade 4 cGVHD. In some embodiments, less than about 1% of subjects that are administered a composition of the disclosure develop ≥grade 4 cGVHD.

Subjects administered a composition of the disclosure (e.g., a cell population as described herein) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop mild to severe cGVHD, for example, within 365 days post-transplant, two years post-transplant, or within another suitable amount of time post-transplant as disclosed herein.

Subjects administered a composition of the disclosure (e.g., a cell population as described herein) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure develop moderate to severe cGVHD. In some embodiments, less than about 40% of subjects that are administered a composition of the disclosure develop moderate to severe cGVHD. In some embodiments, less than about 30% of subjects that are administered a composition of the disclosure develop moderate to severe cGVHD. In some embodiments, less than about 20% of subjects that are administered a composition of the disclosure develop moderate to severe cGVHD. In some embodiments, less than about 10% of subjects that are administered a composition of the disclosure develop moderate to severe cGVHD. In some embodiments, less than about 5% of subjects that are administered a composition of the disclosure develop moderate to severe cGVHD.

Subjects administered a composition of the disclosure (e.g., a cell population as described herein) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop severe cGVHD, for example, within 365 days post-transplant, two years post-transplant, or within another suitable amount of time post-transplant as disclosed herein.

In some embodiments, less than about 50% of subjects that are administered a composition of the disclosure develop severe cGVHD. In some embodiments, less than about 40% of subjects that are administered a composition of the disclosure develop severe cGVHD. In some embodiments, less than about 30% of subjects that are administered a composition of the disclosure develop severe cGVHD. In some embodiments, less than about 20% of subjects that are administered a composition of the disclosure develop severe cGVHD. In some embodiments, less than about 15% of subjects that are administered a composition of the disclosure develop severe cGVHD. In some embodiments, less than about 10% of subjects that are administered a composition of the disclosure develop severe cGVHD. In some embodiments, less than about 5% of subjects that are administered a composition of the disclosure develop severe cGVHD. In some embodiments, less than about 3% of subjects that are administered a composition of the disclosure develop severe cGVHD. In some embodiments, less than about 2% of subjects that are administered a composition of the disclosure develop severe cGVHD. In some embodiments, less than about 1% of subjects that are administered a composition of the disclosure develop severe cGVHD.

The incidence of cGVHD can be assessed after a suitable amount of time elapses post-transplant, for example, about 150 days, about 200 days, about 365 days, about 1.5 years, about 2 years, about 2.5 years, about 3 years, about 3.5 years, about 4 years, about 4.5 years, or about 5 years post-transplant.

The incidence of cGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, subjects administered a composition of the disclosure (e.g., a cell population as described herein) have a decreased wherein incidence, severity, timing, or any combination thereof of Grade I to Grade IV chronic GVHD (cGVHD) or severe cGVHD, relative to a corresponding human subject that has been administered a standard myeloablative allogeneic hematopoietic stem cell transplant (alloHSCT). In some embodiments, the subject is free of Grade I to Grade IV cGVHD or severe cGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have increased cGVHD survival, relative to a corresponding human subject that has been administered a standard myeloablative allogeneic hematopoietic stem cell transplant (alloHSCT). In some embodiments, the subject experiences cGHVD-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

### No GVHID Prophylaxis

Subjects administered a composition of the disclosure (e.g., a cell population as described herein) in the absence of GVHD prophylactic agents exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 2 cGVHD. In some embodiments, less than about 40% of subjects that are administered a composition of the disclosure in the absence of GVHD prophylactic agents develop ≥grade 2 cGVHD. In some embodiments, less than about 30% of subjects that are administered a composition of the disclosure in the absence of GVHD prophylactic agents develop ≥grade 2 cGVHD. In some embodiments, less than about 25% of subjects that are administered a composition of the disclosure in the absence of GVHD prophylactic agents develop ≥grade 2 cGVHD. In some embodiments, less than about 20% of subjects that are administered a composition of the disclosure in the absence of GVHD prophylactic agents develop ≥grade 2 cGVHD. In some embodiments, less than about 10% of subjects that are administered a composition of the disclosure in the absence of GVHD prophylactic agents develop ≥grade 2 cGVHD. In some embodiments, less than about 5% of subjects that are administered a composition of the disclosure in the absence of GVHD prophylactic agents develop ≥grade 2 cGVHD.

Subjects administered a composition of the disclosure (e.g., a cell population as described herein) in the absence of GVHD prophylactic agents exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 3 cGVHD. In some embodiments, less than about 40% of subjects that are administered a composition of the disclosure in the absence of GVHD prophylactic agents develop ≥grade 3 cGVHD. In some embodiments, less than about 30% of subjects that are administered a composition of the disclosure in the absence of GVHD prophylactic agents develop ≥grade 3 cGVHD. In some embodiments, less than about 20% of subjects that are administered a composition of the disclosure in the absence of GVHD prophylactic agents develop ≥grade 3 cGVHD. In some embodiments, less than about 10% of subjects that are administered a composition of the disclosure in the absence of GVHD prophylactic agents develop ≥grade 3 cGVHD. In some embodiments, less than about 5% of subjects that are administered a composition of the disclosure in the absence of GVHD prophylactic agents develop ≥grade 3 cGVHD. In some embodiments, less than about 3% of subjects that are administered a composition of the disclosure in the absence of GVHD prophylactic agents develop ≥grade 3 cGVHD. In some embodiments, less than about 2% of subjects that are administered a composition of the disclosure in the absence of GVHD prophylactic agents develop ≥grade 3 cGVHD. In some embodiments, less than about 1% of subjects that are administered a composition of the disclosure in the absence of GVHD prophylactic agents develop ≥grade 3 cGVHD.

Subjects administered a composition of the disclosure (e.g., a cell population as described herein) in the absence of GVHD prophylactic agents exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 40%, or less than about 50% of subjects that are administered a composition of the disclosure in the absence of GVHD prophylactic agents develop ≥grade 4 cGVHD, for example, within 365 days post-transplant, two years post-transplant, or within another suitable amount of time post-transplant as disclosed herein.

In some embodiments, less than about 30% of subjects that are administered a composition of the disclosure in the absence of GVHD prophylactic agents develop ≥grade 4 cGVHD. In some embodiments, less than about 20% of subjects that are administered a composition of the disclosure in the absence of GVHD prophylactic agents develop ≥grade 4 cGVHD. In some embodiments, less than about 15% of subjects that are administered a composition of the disclosure in the absence of GVHD prophylactic agents develop ≥grade 4 cGVHD. In some embodiments, less than about 10% of subjects that are administered a composition of the disclosure in the absence of GVHD prophylactic agents develop ≥grade 4 cGVHD. In some embodiments, less than about 5% of subjects that are administered a composition of the disclosure in the absence of GVHD prophylactic agents develop ≥grade 4 cGVHD. In some embodiments, less than about 3% of subjects that are administered a composition of the disclosure in the absence of GVHD prophylactic agents develop ≥grade 4 cGVHD. In some embodiments, less than about 2% of subjects that are administered a composition of the disclosure in the absence of GVHD prophylactic agents develop ≥grade 4 cGVHD. In some embodiments, less than about 1% of subjects that are administered a composition of the disclosure in the absence of GVHD prophylactic agents develop ≥grade 4 cGVHD.

Subjects administered a composition of the disclosure (e.g., a cell population as described herein) in the absence of GVHD prophylactic agents exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop mild to severe cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop moderate to severe cGVHD. In some embodiments, less than about 40% of subjects that are administered a composition of the disclosure in the absence of GVHD prophylactic agents develop moderate to severe cGVHD. In some embodiments, less than about 30% of subjects that are administered a composition of the disclosure in the absence of GVHD prophylactic agents develop moderate to severe cGVHD. In some embodiments, less than about 25% of subjects that are administered a composition of the disclosure in the absence of GVHD prophylactic agents develop moderate to severe cGVHD. In some embodiments, less than about 20% of subjects that are administered a composition of the disclosure in the absence of GVHD prophylactic agents develop moderate to severe cGVHD. In some embodiments, less than about 10% of subjects that are administered a composition of the disclosure in the absence of GVHD prophylactic agents develop moderate to severe cGVHD. In some embodiments, less than about 5% of subjects that are administered a composition of the disclosure in the absence of GVHD prophylactic agents develop moderate to severe cGVHD.

Subjects administered a composition of the disclosure (e.g., a cell population as described herein) in the absence of GVHD prophylactic agents exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop severe cGVHD. In some embodiments, less than about 40% of subjects that are administered a composition of the disclosure in the absence of GVHD prophylactic agents develop severe cGVHD. In some embodiments, less than about 30% of subjects that are administered a composition of the disclosure in the absence of GVHD prophylactic agents develop severe cGVHD. In some embodiments, less than about 20% of subjects that are administered a composition of the disclosure in the absence of GVHD prophylactic agents develop severe cGVHD. In some embodiments, less than about 10% of subjects that are administered a composition of the disclosure in the absence of GVHD prophylactic agents develop severe cGVHD. In some embodiments, less than about 5% of subjects that are administered a composition of the disclosure in the absence of GVHD prophylactic agents develop severe cGVHD. In some embodiments, less than about 3% of subjects that are administered a composition of the disclosure in the absence of GVHD prophylactic agents develop severe cGVHD. In some embodiments, less than about 2% of subjects that are administered a composition of the disclosure in the absence of GVHD prophylactic agents develop severe cGVHD. In some embodiments, less than about 1% of subjects that are administered a composition of the disclosure in the absence of GVHD prophylactic agents develop severe cGVHD.

The absence of GVHD prophylactic agents can refer to cases where no GVHD prophylactic agents are administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant.

### Single Agent GVHD Prophylaxis

Subjects administered a composition of the disclosure (e.g., a cell population as described herein) and no more than one GVHD prophylactic agent (for example, a single GVHD prophylactic agent) exhibit a low incidence of ≥grade 1 cGVHD, for example, a lower incidence of ≥grade 1 cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 2 cGVHD. In some embodiments, less than about 40% of subjects that are administered a composition of the disclosure and no more than one GVHD prophylactic agent (for example, a single GVHD prophylactic agent) develop ≥grade 2 cGVHD. In some embodiments, less than about 30% of subjects that are administered a composition of the disclosure and no more than one GVHD prophylactic agent (for example, a single GVHD prophylactic agent) develop ≥grade 2 cGVHD. In some embodiments, less than about 20% of subjects that are administered a composition of the disclosure and no more than one GVHD prophylactic agent (for example, a single GVHD prophylactic agent) develop ≥grade 2 cGVHD. In some embodiments, less than about 10% of subjects that are administered a composition of the disclosure and no more than one GVHD prophylactic agent (for example, a single GVHD prophylactic agent) develop ≥grade 2 cGVHD. In some embodiments, less than about 5% of subjects that are administered a composition of the disclosure and no more than one GVHD prophylactic agent (for example, a single GVHD prophylactic agent) develop ≥grade 2 cGVHD.

Subjects administered a composition of the disclosure (e.g., a cell population as described herein) and no more than one GVHD prophylactic agent (for example, a single GVHD prophylactic agent) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 3 cGVHD. In some embodiments, less than about 40% of subjects that are administered a composition of the disclosure and no more than one GVHD prophylactic agent (for example, a single GVHD prophylactic agent) develop ≥grade 3 cGVHD. In some embodiments, less than about 30% of subjects that are administered a composition of the disclosure and no more than one GVHD prophylactic agent (for example, a single GVHD prophylactic agent) develop ≥grade 3 cGVHD. In some embodiments, less than about 20% of subjects that are administered a composition of the disclosure and no more than one GVHD prophylactic agent (for example, a single GVHD prophylactic agent) develop ≥grade 3 cGVHD. In some embodiments, less than about 15% of subjects that are administered a composition of the disclosure and no more than one GVHD prophylactic agent (for example, a single GVHD prophylactic agent) develop ≥grade 3 cGVHD. In some embodiments, less than about 10% of subjects that are administered a composition of the disclosure and no more than one GVHD prophylactic agent (for example, a single GVHD prophylactic agent) develop ≥grade 3 cGVHD. In some embodiments, less than about 5% of subjects that are administered a composition of the disclosure and no more than one GVHD prophylactic agent (for example, a single GVHD prophylactic agent) develop ≥grade 3 cGVHD. In some embodiments, less than about 3% of subjects that are administered a composition of the disclosure and no more than one GVHD prophylactic agent (for example, a single GVHD prophylactic agent) develop ≥grade 3 cGVHD. In some embodiments, less than about 2% of subjects that are administered a composition of the disclosure and no more than one GVHD prophylactic agent (for example, a single GVHD prophylactic agent) develop ≥grade 3 cGVHD. In some embodiments, less than about 1% of subjects that are administered a composition of the disclosure and no more than one GVHD prophylactic agent (for example, a single GVHD prophylactic agent) develop ≥grade 3 cGVHD.

Subjects administered a composition of the disclosure (e.g., a cell population as described herein) and no more than one GVHD prophylactic agent (for example, a single GVHD prophylactic agent) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and no more than one GVHD prophylactic agent (for example, a single GVHD prophylactic agent) develop ≥grade 4 cGVHD. In some embodiments, less than about 30% of subjects that are administered a composition of the disclosure and no more than one GVHD prophylactic agent (for example, a single GVHD prophylactic agent) develop ≥grade 4 cGVHD. In some embodiments, less than about 20% of subjects that are administered a composition of the disclosure and no more than one GVHD prophylactic agent (for example, a single GVHD prophylactic agent) develop ≥grade 4 cGVHD. In some embodiments, less than about 10% of subjects that are administered a composition of the disclosure and no more than one GVHD prophylactic agent (for example, a single GVHD prophylactic agent) develop ≥grade 4 cGVHD. In some embodiments, less than about 5% of subjects that are administered a composition of the disclosure and no more than one GVHD prophylactic agent (for example, a single GVHD prophylactic agent) develop ≥grade 4 cGVHD. In some embodiments, less than about 3% of subjects that are administered a composition of the disclosure and no more than one GVHD prophylactic agent (for example, a single GVHD prophylactic agent) develop ≥grade 4 cGVHD. In some embodiments, less than about 2% of subjects that are administered a composition of the disclosure and no more than one GVHD prophylactic agent (for example, a single GVHD prophylactic agent) develop ≥grade 4 cGVHD. In some embodiments, less than about 1% of subjects that are administered a composition of the disclosure and no more than one GVHD prophylactic agent (for example, a single GVHD prophylactic agent) develop ≥grade 4 cGVHD.

Subjects administered a composition of the disclosure (e.g., a cell population as described herein) and no more than one GVHD prophylactic agent (for example, a single GVHD prophylactic agent) exhibit a low incidence of mild to severe cGVHD, for example, a lower incidence of mild to severe cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop moderate to severe cGVHD. In some embodiments, less than about 40% of subjects that are administered a composition of the disclosure and no more than one GVHD prophylactic agent (for example, a single GVHD prophylactic agent) develop moderate to severe cGVHD. In some embodiments, less than about 30% of subjects that are administered a composition of the disclosure and no more than one GVHD prophylactic agent (for example, a single GVHD prophylactic agent) develop moderate to severe cGVHD. In some embodiments, less than about 20% of subjects that are administered a composition of the disclosure and no more than one GVHD prophylactic agent (for example, a single GVHD prophylactic agent) develop moderate to severe cGVHD. In some embodiments, less than about 10% of subjects that are administered a composition of the disclosure and no more than one GVHD prophylactic agent (for example, a single GVHD prophylactic agent) develop moderate to severe cGVHD. In some embodiments, less than about 5% of subjects that are administered a composition of the disclosure and no more than one GVHD prophylactic agent (for example, a single GVHD prophylactic agent) develop moderate to severe cGVHD.

Subjects administered a composition of the disclosure (e.g., a cell population as described herein) and no more than one GVHD prophylactic agent (for example, a single GVHD prophylactic agent) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop severe cGVHD. In some embodiments, less than about 40% of subjects that are administered a composition of the disclosure and no more than one GVHD prophylactic agent (for example, a single GVHD prophylactic agent) develop severe cGVHD. In some embodiments, less than about 30% of subjects that are administered a composition of the disclosure and no more than one GVHD prophylactic agent (for example, a single GVHD prophylactic agent) develop severe cGVHD. In some embodiments, less than about 20% of subjects that are administered a composition of the disclosure and no more than one GVHD prophylactic agent (for example, a single GVHD prophylactic agent) develop severe cGVHD. In some embodiments, less than about 15% of subjects that are administered a composition of the disclosure and no more than one GVHD prophylactic agent (for example, a single GVHD prophylactic agent) develop severe cGVHD. In some embodiments, less than about 10% of subjects that are administered a composition of the disclosure and no more than one GVHD prophylactic agent (for example, a single GVHD prophylactic agent) develop severe cGVHD. In some embodiments, less than about 5% of subjects that are administered a composition of the disclosure and no more than one GVHD prophylactic agent (for example, a single GVHD prophylactic agent) develop severe cGVHD. In some embodiments, less than about 3% of subjects that are administered a composition of the disclosure and no more than one GVHD prophylactic agent (for example, a single GVHD prophylactic agent) develop severe cGVHD. In some embodiments, less than about 2% of subjects that are administered a composition of the disclosure and no more than one GVHD prophylactic agent (for example, a single GVHD prophylactic agent) develop severe cGVHD. In some embodiments, less than about 1% of subjects that are administered a composition of the disclosure and no more than one GVHD prophylactic agent (for example, a single GVHD prophylactic agent) develop severe cGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 2 cGVHD. In some embodiments, less than about 40% of subjects that are administered a composition of the disclosure and a low dose of a GVHD prophylactic agent (for example, a single GVHD prophylactic agent at a low dose) develop ≥grade 2 cGVHD. In some embodiments, less than about 30% of subjects that are administered a composition of the disclosure and a low dose of a GVHD prophylactic agent (for example, a single GVHD prophylactic agent at a low dose) develop ≥grade 2 cGVHD. In some embodiments, less than about 20% of subjects that are administered a composition of the disclosure and a low dose of a GVHD prophylactic agent (for example, a single GVHD prophylactic agent at a low dose) develop ≥grade 2 cGVHD. In some embodiments, less than about 15% of subjects that are administered a composition of the disclosure and a low dose of a GVHD prophylactic agent (for example, a single GVHD prophylactic agent at a low dose) develop ≥grade 2 cGVHD. In some embodiments, less than about 10% of subjects that are administered a composition of the disclosure and a low dose of a GVHD prophylactic agent (for example, a single GVHD prophylactic agent at a low dose) develop ≥grade 2 cGVHD. In some embodiments, less than about 5% of subjects that are administered a composition of the disclosure and a low dose of a GVHD prophylactic agent (for example, a single GVHD prophylactic agent at a low dose) develop ≥grade 2 cGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 3 cGVHD. In some embodiments, less than about 40% of subjects that are administered a composition of the disclosure and a low dose of a GVHD prophylactic agent (for example, a single GVHD prophylactic agent at a low dose) develop ≥grade 3 cGVHD. In some embodiments, less than about 30% of subjects that are administered a composition of the disclosure and a low dose of a GVHD prophylactic agent (for example, a single GVHD prophylactic agent at a low dose) develop ≥grade 3 cGVHD. In some embodiments, less than about 20% of subjects that are administered a composition of the disclosure and a low dose of a GVHD prophylactic agent (for example, a single GVHD prophylactic agent at a low dose) develop ≥grade 3 cGVHD. In some embodiments, less than about 15% of subjects that are administered a composition of the disclosure and a low dose of a GVHD prophylactic agent (for example, a single GVHD prophylactic agent at a low dose) develop ≥grade 3 cGVHD. In some embodiments, less than about 10% of subjects that are administered a composition of the disclosure and a low dose of a GVHD prophylactic agent (for example, a single GVHD prophylactic agent at a low dose) develop ≥grade 3 cGVHD. In some embodiments, less than about 5% of subjects that are administered a composition of the disclosure and a low dose of a GVHD prophylactic agent (for example, a single GVHD prophylactic agent at a low dose) develop ≥grade 3 cGVHD. In some embodiments, less than about 3% of subjects that are administered a composition of the disclosure and a low dose of a GVHD prophylactic agent (for example, a single GVHD prophylactic agent at a low dose) develop ≥grade 3 cGVHD. In some embodiments, less than about 2% of subjects that are administered a composition of the disclosure and a low dose of a GVHD prophylactic agent (for example, a single GVHD prophylactic agent at a low dose) develop ≥grade 3 cGVHD. In some embodiments, less than about 1% of subjects that are administered a composition of the disclosure and a low dose of a GVHD prophylactic agent (for example, a single GVHD prophylactic agent at a low dose) develop ≥grade 3 cGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and a low dose of a GVHD prophylactic agent (for example, a single GVHD prophylactic agent at a low dose) develop ≥grade 4 cGVHD. In some embodiments, less than about 30% of subjects that are administered a composition of the disclosure and a low dose of a GVHD prophylactic agent (for example, a single GVHD prophylactic agent at a low dose) develop ≥grade 4 cGVHD. In some embodiments, less than about 20% of subjects that are administered a composition of the disclosure and a low dose of a GVHD prophylactic agent (for example, a single GVHD prophylactic agent at a low dose) develop ≥grade 4 cGVHD. In some embodiments, less than about 10% of subjects that are administered a composition of the disclosure and a low dose of a GVHD prophylactic agent (for example, a single GVHD prophylactic agent at a low dose) develop ≥grade 4 cGVHD. In some embodiments, less than about 5% of subjects that are administered a composition of the disclosure and a low dose of a GVHD prophylactic agent (for example, a single GVHD prophylactic agent at a low dose) develop ≥grade 4 cGVHD. In some embodiments, less than about 3% of subjects that are administered a composition of the disclosure and a low dose of a GVHD prophylactic agent (for example, a single GVHD prophylactic agent at a low dose) develop ≥grade 4 cGVHD. In some embodiments, less than about 2% of subjects that are administered a composition of the disclosure and a low dose of a GVHD prophylactic agent (for example, a single GVHD prophylactic agent at a low dose) develop ≥grade 4 cGVHD. In some embodiments, less than about 1% of subjects that are administered a composition of the disclosure and a low dose of a GVHD prophylactic agent (for example, a single GVHD prophylactic agent at a low dose) develop ≥grade 4 cGVHD.

Subjects administered a composition of the disclosure (e.g., a cell population as described herein) and a low dose of a GVHD prophylactic agent (for example, a single GVHD prophylactic agent at a low dose) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop moderate to severe cGVHD. In some embodiments, less than about 40% of subjects that are administered a composition of the disclosure and a low dose of a GVHD prophylactic agent (for example, a single GVHD prophylactic agent at a low dose) develop moderate to severe cGVHD. In some embodiments, less than about 30% of subjects that are administered a composition of the disclosure and a low dose of a GVHD prophylactic agent (for example, a single GVHD prophylactic agent at a low dose) develop moderate to severe cGVHD. In some embodiments, less than about 20% of subjects that are administered a composition of the disclosure and a low dose of a GVHD prophylactic agent (for example, a single GVHD prophylactic agent at a low dose) develop moderate to severe cGVHD. In some embodiments, less than about 15% of subjects that are administered a composition of the disclosure and a low dose of a GVHD prophylactic agent (for example, a single GVHD prophylactic agent at a low dose) develop moderate to severe cGVHD. In some embodiments, less than about 10% of subjects that are administered a composition of the disclosure and a low dose of a GVHD prophylactic agent (for example, a single GVHD prophylactic agent at a low dose) develop moderate to severe cGVHD. In some embodiments, less than about 5% of subjects that are administered a composition of the disclosure and a low dose of a GVHD prophylactic agent (for example, a single GVHD prophylactic agent at a low dose) develop moderate to severe cGVHD.

Subjects administered a composition of the disclosure (e.g., a cell population as described herein) and a low dose of a GVHD prophylactic agent (for example, a single GVHD prophylactic agent at a low dose) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop severe cGVHD. In some embodiments, less than about 40% of subjects that are administered a composition of the disclosure and a low dose of a GVHD prophylactic agent (for example, a single GVHD prophylactic agent at a low dose) develop severe cGVHD. In some embodiments, less than about 30% of subjects that are administered a composition of the disclosure and a low dose of a GVHD prophylactic agent (for example, a single GVHD prophylactic agent at a low dose) develop severe cGVHD. In some embodiments, less than about 20% of subjects that are administered a composition of the disclosure and a low dose of a GVHD prophylactic agent (for example, a single GVHD prophylactic agent at a low dose) develop severe cGVHD. In some embodiments, less than about 15% of subjects that are administered a composition of the disclosure and a low dose of a GVHD prophylactic agent (for example, a single GVHD prophylactic agent at a low dose) develop severe cGVHD. In some embodiments, less than about 10% of subjects that are administered a composition of the disclosure and a low dose of a GVHD prophylactic agent (for example, a single GVHD prophylactic agent at a low dose) develop severe cGVHD. In some embodiments, less than about 5% of subjects that are administered a composition of the disclosure and a low dose of a GVHD prophylactic agent (for example, a single GVHD prophylactic agent at a low dose) develop severe cGVHD. In some embodiments, less than about 3% of subjects that are administered a composition of the disclosure and a low dose of a GVHD prophylactic agent (for example, a single GVHD prophylactic agent at a low dose) develop severe cGVHD. In some embodiments, less than about 2% of subjects that are administered a composition of the disclosure and a low dose of a GVHD prophylactic agent (for example, a single GVHD prophylactic agent at a low dose) develop severe cGVHD. In some embodiments, less than about 1% of subjects that are administered a composition of the disclosure and a low dose of a GVHD prophylactic agent (for example, a single GVHD prophylactic agent at a low dose) develop severe cGVHD.

A low dose of a GVHD prophylactic agent can be, for example a target trough level of less than about 25 ng/mL, less than about 20 ng/mL, less than about 15 ng/ml, less than about 12 ng/mL, less than about 11 ng/ml, less than about 10 ng/mL, less than about 9 ng/ml, less than about 8 ng/mL, less than about 7 ng/ml, less than about 6 ng/ml, less than about 5 ng/ml, less than about 4 ng/mL, less than about 3 ng/mL, less than about 2 ng/mL, or less than about 1 ng/mL.

In some embodiments, a low dose of a GVHD prophylactic is a target trough level of approximately 1-25 ng/ml, about 1-20 ng/mL, about 1-15 ng/ml, about 1-12 ng/ml, about 1-11 ng/mL, about 1-10 ng/ml, about 1-9 ng/mL, about 1-8 ng/ml, about 1-7 ng/mL, about 1-6 ng/ml, about 1-5 ng/mL, about 1-4 ng/ml, about 1-3 ng/ml, about 1-2 ng/mL, about 2-25 ng/ml, about 2-20 ng/mL, about 2-15 ng/mL, about 2-12 ng/ml, about 2-11 ng/ml, about 2-10 ng/ml, about 2-9 ng/ml, about 2-8 ng/mL, about 2-7 ng/ml, about 2-6 ng/ml, about 2-5 ng/ml, about 2-4 ng/mL, about 2-3 ng/mL, about 3-25 ng/ml, about 3-20 ng/ml, about 3-15 ng/ml, about 3-12 ng/ml, about 3-11 ng/ml, about 3-10 ng/mL, about 3-9 ng/ml, about 3-8 ng/ml, about 3-7 ng/ml, about 3-6 ng/ml, about 3-5 ng/mL, about 3-4 ng/mL, about 4-25 ng/ml, about 4-20 ng/mL, about 4-15 ng/ml, about 4-12 ng/mL, about 4-11 ng/mL, about 4-10 ng/ml, about 4-9 ng/ml, about 4-8 ng/mL, about 4-7 ng/mL, about 4-6 ng/ml, about 4-5 ng/ml, about 5-25 ng/ml, about 5-20 ng/ml, about 5-15 ng/mL, about 5-12 ng/ml, about 5-11 ng/mL, about 5-10 ng/ml, about 5-9 ng/mL, about 5-8 ng/ml, about 5-7 ng/ml, about 5-6 ng/ml, about 6-25 g/mL, about 6-20 ng/mL, about 6-15 ng/mL, about 6-12 ng/ml, about 6-11 ng/ml, about 6-10 ng/ml, about 6-9 ng/ml, about 6-8 ng/ml, about 6-7 ng/ml, about 8-25 ng/ml, about 8-20 ng/ml, about 8-15 ng/mL, about 8-12 ng/ml, about 8-11 ng/ml, about 8-10 ng/ml, about 8-9 ng/mL, about 10-25 ng/mL, about 10-20 ng/ml, about 10-15 ng/ml, about 10-12 ng/ml, or about 10-11 ng/mL.

In some embodiments, a low dose of a GVHD prophylactic agent is tacrolimus with a target trough level of approximately 5 ng/mL to approximately 10 ng/mL. In some embodiments, a low dose of a GVHD prophylactic agent is tacrolimus with a target trough level of approximately 4 ng/ml to approximately 6 ng/mL.

In some embodiments, a low dose of a GVHD prophylactic agent is sirolimus with a target trough level of approximately 3 ng/mL to approximately 8 ng/mL. In some embodiments, a low dose of a GVHD prophylactic agent is sirolimus with a target trough level of approximately 4 ng/ml to approximately 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Organ-Specific GVHD Stage Incidence

Subjects administered a composition of the disclosure (e.g., a cell population as described herein) exhibit a low incidence of ≥stage 1 GVHD signs for the skin, liver, gut, or a combination thereof, for example, a lower incidence compared to subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure exhibit ≥stage 1 GVHD signs for the skin, liver, gut, or a combination thereof.

Subjects administered a composition of the disclosure (e.g., a cell population as described herein) exhibit a low incidence of ≥stage 2 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 1 GVHD signs for the skin, liver, gut, or a combination thereof.

Subjects administered a composition of the disclosure (e.g., a cell population as described herein) exhibit a low incidence of ≥stage 3 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 3 GVHD signs for the skin, liver, gut, or a combination thereof.

Subjects administered a composition of the disclosure (e.g., a cell population as described herein) exhibit a low incidence of ≥stage 4 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 4 GVHD signs for the skin, liver, gut, or a combination thereof.

The incidence of the organ-specific GVHD signs can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days, about 150 days, about 200 days, about 365 days, about 1.5 years, about 2 years, about 2.5 years, about 3 years, about 3.5 years, about 4 years, about 4.5 years, or about 5 years post-transplant.

The incidence of the organ-specific GVHD signs can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### Other Clinical Outcomes

### Survival, Hospitalization, and Relapse

In some embodiments, patient treated or provided with compositions, multi-component pharmaceutical treatments, multi-component cellular therapy products, cell populations, solutions, formulations, kits, and/or methods has a reduced risk of at least one of malignancy relapse, infection or renal failure.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a high overall survival rate, for example, a higher overall survival rate compared to subjects that are administered an alternate composition. In some embodiments, at least about 40%, at least about 50%, at least about 55%, at least about 60%, at least about 65%, at least about 70%, at least about 75%, at least about 80%, at least about 85%, at least about 90%, or at least about 95% of subjects that are administered a composition of the disclosure are alive after about 1 year, about 1.5 years, about 2 years, about 2.5 years, about 3 years, about 3.5 years, about 4 years, about 4.5 years, about 5 years, about 7 years, about 10 years, about 15 years, about 20 years, about 30 years post-transplant.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a low treatment-associated mortality rate, for example, a lower treatment-associated mortality rate compared to subjects that are administered an alternate composition. In some embodiments, a treatment-associated mortality rate of a population of subjects administered a composition of the disclosure is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 25%, less than about 30%, less than about 35%, less than about 40%, or less than about 50% when evaluated after a suitable amount of time post-transplant, for example at approximately 6 months, about 1 year, about 1.5 years, about 2 years, about 2.5 years, about 3 years, about 3.5 years, about 4 years, about 4.5 years, or about 5 years post-transplant. In some embodiments a treatment-associated mortality rate of a population of subjects administered a composition of the disclosure is less than about 5% when evaluated at 1-year post-transplant. In some embodiments a treatment-associated mortality rate of a population of subjects administered a composition of the disclosure is less than about 1% when evaluated at 1-year post-transplant.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit GVHD free and relapse free survival (GRFS) rate, for example, a higher GRFS rate compared to subjects that are administered an alternate composition. In some embodiments, GRFS can refer to survival without relapse or Grade ≥3 aGVHD or extensive (e.g., severe) cGVHD. In some embodiments, GRFS can refer to survival without relapse or Grade ≥2 aGVHD or extensive (e.g., moderate to severe) cGVHD. In some embodiments, GRFS can refer to survival with no GVHD symptoms. In some embodiments, a GRFS rate of a population of subjects administered a composition of the disclosure is at least about 20%, at least about 30%, at least about 40%, at least about 50%, at least about 55%, at least about 60%, at least about 65%, at least about 70%, at least about 75%, at least about 80%, at least about 85%, at least about 90%, or at least about 95% when evaluated after a suitable amount of time post-transplant, for example at approximately 6 months, about 1 year, about 1.5 years, about 2 years, about 2.5 years, about 3 years, about 3.5 years, about 4 years, about 4.5 years, or about 5 years post-transplant. In some embodiments, a GRFS rate of a population of subjects administered a composition of the disclosure is at least about 60% when evaluated at 1-year post-transplant. In some embodiments, a GRFS rate of a population of subjects administered a composition of the disclosure is at least about 75% when evaluated at 1-year post-transplant. In some embodiments, a GRFS rate of a population of subjects administered a composition of the disclosure is more than 75% when evaluated at 3 years post-transplant. In some embodiments, a GRFS rate of a population of subjects administered a composition of the disclosure is more than 75% when evaluated at 5 years post-transplant. In some embodiments, a GRFS rate of a population of subjects administered a composition of the disclosure is more than 75% when evaluated at 7 years post-transplant. In some embodiments, a GRFS rate of a population of subjects administered a composition of the disclosure is more than 75% when evaluated at 10 years post-transplant.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a short time to discharge from hospital, for example, a shorter time to discharge from hospital compared to subjects that are administered an alternate composition. In some embodiments, the average time to discharge after day 0 of a transplantation regimen is less than about 20 days, less than about 19 days, less than about 18 days, less than about 17 days, less than about 16 days, less than about 15 days, less than about 14 days, less than about 13 days, less than about 12 days, less than about 11 days, less than about 10 days, less than about 9 days, or less than about 8 days. In some embodiments, the average time to discharge after day 0 of a transplantation regimen is less than about 17 days. In some embodiments, the average time to discharge after day 0 of a transplantation regimen is less than about 18 days. In embodiments, the human subject treated or provided with compositions, multi-component pharmaceutical treatments, multi-component cellular therapy products, cell populations, solutions, formulations, kits, and/or methods as disclosed herein have no relapse of their malignancy about one year after being administered the pharmaceutical dosing regimen.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a low relapse rate, for example, a lower relapse rate compared to subjects that are administered an alternate composition. In some embodiments, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that are administered a composition of the disclosure relapse within a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 35% of subjects that are administered a composition of the disclosure relapse within 1-year post-transplant. In some embodiments, less than about 25% of subjects that are administered a composition of the disclosure relapse within 1-year post-transplant.

A population of subjects that do not have active disease when treated or provided with compositions, multi-component pharmaceutical treatments, multi-component cellular therapy products, cell populations, solutions, formulations, kits, and/or methods of the disclosure (e.g., a cell population as described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that do not have active disease when administered a composition of the disclosure relapse within a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient treated or provided with compositions, multi-component pharmaceutical treatments, multi-component cellular therapy products, cell populations, solutions, formulations, kits, and/or methods has no relapse of their malignancy about one year after being administered the cell populations.

A population of subjects that are in complete remission when administered a composition of the disclosure (e.g., a cell population as described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, or less than about 40% of subjects that are in complete remission when administered a composition of the disclosure relapse within a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient has no GHVD or relapse of their malignancy one year after being administered the cell populations.

In some embodiments, subjects administered a composition of the disclosure (e.g., a cell population as described herein) experience increased GVHD and relapse-free survival (GFRS), relative to a corresponding human subject that has been administered a standard myeloablative allogeneic hematopoietic stem cell transplant (alloHSCT). In some embodiments, GFRS is increased by at least approximately 0.05-fold, at least approximately 0.1-fold, at least approximately 0.15-fold, at least approximately 0.2-fold, at least approximately 0.25-fold, at least approximately 0.3-fold, at least approximately 0.35-fold, at least approximately 0.4-fold, at least approximately 0.45-fold, at least approximately 0.5-fold, at least approximately 0.55-fold, at least approximately 0.6-fold, at least approximately 0.65-fold, at least approximately 0.7-fold, at least approximately 0.75-fold, at least approximately 0.8-fold, at least approximately 0.85-fold, at least approximately 0.9-fold, at least approximately 0.95-fold, at least approximately 1-fold, at least approximately 1.05-fold, at least approximately 1.1-fold, at least approximately 1.15-fold, at least approximately 1.2-fold, at least approximately 1.25-fold, at least approximately 1.3-fold, at least approximately 1.35-fold, at least approximately 1.4-fold, at least approximately 1.45-fold, at least approximately 1.5-fold, at least approximately 1.55-fold, at least approximately 1.6-fold, at least approximately 1.65-fold, at least approximately 1.7-fold, at least approximately 1.75-fold, at least approximately 1.8-fold, at least approximately 1.85-fold, at least approximately 1.9-fold, at least approximately 1.95-fold, at least approximately 2-fold, at least approximately 2.05-fold, at least approximately 2.1-fold, at least approximately 2.15-fold, at least approximately 2.2-fold, at least approximately 2.25-fold, at least approximately 2.3-fold, at least approximately 2.35-fold, at least approximately 2.4-fold, at least approximately 2.45-fold, at least approximately 2.5-fold, at least approximately 2.55-fold, at least approximately 2.6-fold, at least approximately 2.65-fold, at least approximately 2.7-fold, at least approximately 2.75-fold, at least approximately 2.8-fold, at least approximately 2.85-fold, at least approximately 2.9-fold, at least approximately 2.95-fold, at least approximately 3-fold, at least approximately 3.05-fold, at least approximately 3.1-fold, at least approximately 3.15-fold, at least approximately 3.2-fold, at least approximately 3.25-fold, at least approximately 3.3-fold, at least approximately 3.35-fold, at least approximately 3.4-fold, at least approximately 3.45-fold, at least approximately 3.5-fold, at least approximately 3.55-fold, at least approximately 3.6-fold, at least approximately 3.65-fold, at least approximately 3.7-fold, at least approximately 3.75-fold, at least approximately 3.8-fold, at least approximately 3.85-fold, at least approximately 3.9-fold, at least approximately 3.95-fold, at least approximately 4-fold, at least approximately 4.05-fold, at least approximately 4.1-fold, at least approximately 4.15-fold, at least approximately 4.2-fold, at least approximately 4.25-fold, at least approximately 4.3-fold, at least approximately 4.35-fold, at least approximately 4.4-fold, at least approximately 4.45-fold, at least approximately 4.5-fold, at least approximately 4.55-fold, at least approximately 4.6-fold, at least approximately 4.65-fold, at least approximately 4.7-fold, at least approximately 4.75-fold, at least approximately 4.8-fold, at least approximately 4.85-fold, at least approximately 4.9-fold, at least approximately 4.95-fold, or at least 5-fold.

In some embodiments, subjects administered a composition of the disclosure (e.g., a cell population as described herein) the subject experiences GFRS for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, GFRS can refer to survival free of death from any cause, relapse, Grade 3-4 aGVHD (e.g., graded per MAGIC), and moderate to severe cGVHD (e.g., graded per NIH consensus criteria). In some embodiments, relapse for acute leukemia can refer to ≥5% blasts in the bone marrow or peripheral blood, reappearance of pre-transplant cytogenetic abnormality, and/or new evidence or redevelopment of extramedullary disease. In some embodiments, relapse for MDS can refer to satisfying criteria for evolution into acute leukemia, reappearance of pre-transplant morphologic abnormalities, detected in bone marrow specimens, and/or reappearance of pre-transplant cytogenetic abnormality in at least one metaphase on each of two separate consecutive examinations at least one month apart, regardless of the number of metaphases analyzed.

In some embodiments, relapse for CML can refer to the emergence of BCR-ABL positivity and/or cytogenetic relapse that requires the institution of secondary therapy for CML. In some embodiments, this can include the use of donor lymphocyte infusion, tyrosine kinase inhibitors (TKIs), or chemotherapy in response to molecular and/or cytogenetic progression.

In some embodiments, subjects administered a composition of the disclosure (e.g., a cell population as described herein) experience increased relapse-free survival, relative to a corresponding human subject that has been administered a standard myeloablative allogeneic hematopoietic stem cell transplant (alloHSCT). In some embodiments, the subject experiences relapse-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) experience increased overall survival, relative to a corresponding human subject that has been administered a standard myeloablative allogeneic hematopoietic stem cell transplant (alloHSCT). In some embodiments, overall survival is increased by at least approximately 0.05-fold, at least approximately 0.1-fold, at least approximately 0.15-fold, at least approximately 0.2-fold, at least approximately 0.25-fold, at least approximately 0.3-fold, at least approximately 0.35-fold, at least approximately 0.4-fold, at least approximately 0.45-fold, at least approximately 0.5-fold, at least approximately 0.55-fold, at least approximately 0.6-fold, at least approximately 0.65-fold, at least approximately 0.7-fold, at least approximately 0.75-fold, at least approximately 0.8-fold, at least approximately 0.85-fold, at least approximately 0.9-fold, at least approximately 0.95-fold, at least approximately 1-fold, at least approximately 1.05-fold, at least approximately 1.1-fold, at least approximately 1.15-fold, at least approximately 1.2-fold, at least approximately 1.25-fold, at least approximately 1.3-fold, at least approximately 1.35-fold, at least approximately 1.4-fold, at least approximately 1.45-fold, at least approximately 1.5-fold, at least approximately 1.55-fold, at least approximately 1.6-fold, at least approximately 1.65-fold, at least approximately 1.7-fold, at least approximately 1.75-fold, at least approximately 1.8-fold, at least approximately 1.85-fold, at least approximately 1.9-fold, at least approximately 1.95-fold, at least approximately 2-fold, at least approximately 2.05-fold, at least approximately 2.1-fold, at least approximately 2.15-fold, at least approximately 2.2-fold, at least approximately 2.25-fold, at least approximately 2.3-fold, at least approximately 2.35-fold, at least approximately 2.4-fold, at least approximately 2.45-fold, at least approximately 2.5-fold, at least approximately 2.55-fold, at least approximately 2.6-fold, at least approximately 2.65-fold, at least approximately 2.7-fold, at least approximately 2.75-fold, at least approximately 2.8-fold, at least approximately 2.85-fold, at least approximately 2.9-fold, at least approximately 2.95-fold, at least approximately 3-fold, at least approximately 3.05-fold, at least approximately 3.1-fold, at least approximately 3.15-fold, at least approximately 3.2-fold, at least approximately 3.25-fold, at least approximately 3.3-fold, at least approximately 3.35-fold, at least approximately 3.4-fold, at least approximately 3.45-fold, at least approximately 3.5-fold, at least approximately 3.55-fold, at least approximately 3.6-fold, at least approximately 3.65-fold, at least approximately 3.7-fold, at least approximately 3.75-fold, at least approximately 3.8-fold, at least approximately 3.85-fold, at least approximately 3.9-fold, at least approximately 3.95-fold, at least approximately 4-fold, at least approximately 4.05-fold, at least approximately 4.1-fold, at least approximately 4.15-fold, at least approximately 4.2-fold, at least approximately 4.25-fold, at least approximately 4.3-fold, at least approximately 4.35-fold, at least approximately 4.4-fold, at least approximately 4.45-fold, at least approximately 4.5-fold, at least approximately 4.55-fold, at least approximately 4.6-fold, at least approximately 4.65-fold, at least approximately 4.7-fold, at least approximately 4.75-fold, at least approximately 4.8-fold, at least approximately 4.85-fold, at least approximately 4.9-fold, at least approximately 4.95-fold, or at least 5-fold.

In some embodiments, the subject experiences overall survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have a decreased incidence, severity, timing, or any combination thereof of non-relapse mortality, relative to a corresponding human subject that has been administered a standard myeloablative allogeneic hematopoietic stem cell transplant (alloHSCT). In some embodiments, incidence of non-relapse mortality is decreased by at least approximately 5%, least approximately 6%, least approximately 7%, least approximately 8%, least approximately 9%, by at least approximately 10%, by at least approximately 11%, by at least approximately 12%, by at least approximately 13%, by at least approximately 14%, by at least approximately 15%, by at least approximately 16%, by at least approximately 17%, by at least approximately 18%, by at least approximately 19%, by at least approximately 20 by at least approximately 21%, by at least approximately 22%, by at least approximately 23%, by at least approximately 24%, by at least approximately 25%, by at least approximately 26%, by at least approximately 27%, by at least approximately 28%, by at least approximately 29%, by at least approximately 30%, by at least approximately 31%, by at least approximately 32%, by at least approximately 33%, by at least approximately 34%, by at least approximately 35%, by at least approximately 36%, by at least approximately 37%, by at least approximately 38%, by at least approximately 39%, by at least approximately 40%, by at least approximately 41%, by at least approximately 42%, by at least approximately 43%, by at least approximately 44%, by at least approximately 45%, by at least approximately 46%, by at least approximately 47%, by at least approximately 48%, by at least approximately 49%, by at least approximately 50%, by at least approximately 51%, by at least approximately 52%, by at least approximately 53%, by at least approximately 54%, by at least approximately 55%, by at least approximately 56%, by at least approximately 57%, by at least approximately 58%, by at least approximately 59%, by at least approximately 60%, by at least approximately 61%, by at least approximately 62%, by at least approximately 63%, by at least approximately 64%, by at least approximately 65%, by at least approximately 66%, by at least approximately 67%, by at least approximately 68%, by at least approximately 69%, by at least approximately 70%, by at least approximately 71%, by at least approximately 72%, by at least approximately 73%, by at least approximately 74%, by at least approximately 75%, by at least approximately 76%, by at least approximately 77%, by at least approximately 78%, by at least approximately 79%, by at least approximately 80%, by at least approximately 81%, by at least approximately 82%, by at least approximately 83%, by at least approximately 84%, by at least approximately 85%, by at least approximately 86%, by at least approximately 87%, by at least approximately 88%, by at least approximately 89%, by at least approximately 90%, by at least approximately 91%, by at least approximately 92%, by at least approximately 93%, by at least approximately 94%, by at least approximately 95%, by at least approximately 96%, by at least approximately 97%, by at least approximately 98%, or by at least approximately 99%. In some embodiments, non-relapse mortality can refer to death without evidence of disease recurrence.

In some embodiments, the subject does not experience non-relapse mortality for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have decreased incidence of Grade 3 or higher infections, relative to a corresponding human subject that has been administered a standard myeloablative allogeneic hematopoietic stem cell transplant (alloHSCT). In some embodiments, the subject is free of Grade 3 or higher infections for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure. In some embodiments, the Grade 3 or higher infection is graded using the BMT-CTN MOP V4.0 grading scale.

In some embodiments, subjects administered a composition of the disclosure (e.g., a cell population as described herein) have a decreased incidence of re-hospitalization, relative to a corresponding human subject that has been administered a standard myeloablative allogeneic hematopoietic stem cell transplant (alloHSCT). In some embodiments, the subject does not require re-hospitalization for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

### Engraftment and Immune Reconstitution

### Graft Failure

A population of subjects that are administered a composition of the disclosure (e.g., a cell population as described herein) exhibit a low rate of primary graft failure, for example, a lower rate of primary graft failure compared to subjects that are administered an alternate composition. Primary graft failure can be a failure to achieve an absolute neutrophil count of >500 cells/uL after Day 30 post-transplant. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit primary graft failure. In some embodiments, less than about 1% of subjects administered a composition of the disclosure exhibit primary graft failure. In some embodiments, less than about 3% of subjects administered a composition of the disclosure exhibit primary graft failure.

A population of subjects that are administered a composition of the disclosure (e.g., a cell population as described herein) exhibit a low rate of secondary graft failure, for example, a lower rate of secondary graft failure compared to subjects that are administered an alternate composition. Secondary graft failure can be a sustained loss of hematopoiesis after engraftment. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit secondary graft failure when evaluated a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 1% of subjects administered a composition of the disclosure exhibit secondary graft failure within 1-year post-transplant. In some embodiments, less than about 5% of subjects administered a composition of the disclosure exhibit secondary graft failure within 1-year post-transplant.

In some embodiments, subjects administered a composition of the disclosure (e.g., a cell population as described herein) have a decreased wherein incidence of primary graft failure or secondary graft failure, relative to a corresponding human subject that has been administered a standard myeloablative allogeneic hematopoietic stem cell transplant (alloHSCT). In some embodiments, the subject is free of primary graft failure or secondary graft failure for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

### Neutrophil Engraftment

A population of subjects that are administered a composition of the disclosure (e.g., a cell population as described herein) exhibit fast neutrophil engraftment, for example, faster neutrophil engraftment compared to subjects that are administered an alternate composition. Neutrophil engraftment can be indicated by a sustained neutrophil count of >500 cells/uL in the peripheral blood of the recipient. In some embodiments, a population of subjects administered a composition of the disclosure achieve neutrophil engraftment by a median of approximately 7 days, about 8 days, about 9 days, about 10 days, about 11 days, about 12 days, about 12 days, about 14 days, or about 15 days post-transplant. In some embodiments, a population of subjects administered a composition of the disclosure achieve neutrophil engraftment by a median of 13 days post-transplant. In some embodiments, a population of subjects administered a composition of the disclosure achieve neutrophil engraftment by a median of 12 days post-transplant. In some embodiments, a population of subjects administered a composition of the disclosure achieve neutrophil engraftment by a median of 11 days post-transplant.

In some embodiments, subjects administered a composition of the disclosure (e.g., a cell population as described herein) have increased incidence, timing, or both incidence and timing of neutrophil engraftment, relative to a corresponding human subject that has been administered a standard myeloablative allogeneic hematopoietic stem cell transplant (alloHSCT). In some embodiments, neutrophil engraftment occurs approximately 1 day or more, approximately 2 days or more, approximately 3 days or more, approximately 4 days or more, approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 21 days or more, approximately 22 days or more, approximately 23 days or more, approximately 24 days or more, approximately 25 days or more, approximately 26 days or more, approximately 27 days or more, approximately 28 days or more, approximately 29 days or more, approximately 30 days or more, approximately 31 days or more, approximately 32 days or more, approximately 33 days or more, approximately 34 days or more, approximately 35 days or more, approximately 36 days or more, approximately 37 days or more, approximately 38 days or more, approximately 39 days or more, approximately 40 days or more, approximately 41 days or more, approximately 42 days or more, approximately 43 days or more, approximately 44 days or more, approximately 45 days or more, approximately 46 days or more, approximately 47 days or more, approximately 48 days or more, approximately 49 days or more, approximately 50 days or more, approximately 51 days or more, approximately 52 days or more, approximately 53 days or more, approximately 54 days or more, approximately 55 days or more, approximately 56 days or more, approximately 57 days or more, approximately 58 days or more, approximately 59 days or more, approximately 60 days or more, approximately 61 days or more, approximately 62 days or more, approximately 63 days or more, approximately 64 days or more, approximately 65 days or more, approximately 66 days or more, approximately 67 days or more, approximately 68 days or more, approximately 69 days or more, approximately 70 days or more, approximately 71 days or more, approximately 72 days or more, approximately 73 days or more, approximately 74 days or more, approximately 75 days or more, approximately 76 days or more, approximately 77 days or more, approximately 78 days or more, approximately 79 days or more, approximately 80 days or more, approximately 81 days or more, approximately 82 days or more, approximately 83 days or more, approximately 84 days or more, approximately 85 days or more, approximately 86 days or more, approximately 87 days or more, approximately 88 days or more, approximately 89 days or more, or 90 days or more after administration of the composition of the disclosure.

### Platelet Engraftment

A population of subjects that are administered a composition of the disclosure (e.g., a cell population comprising a population of cells described herein) exhibit fast platelet engraftment, for example, faster platelet engraftment compared to subjects that are administered an alternate composition. Platelet engraftment can be indicated by a platelet count >20,000/mm³ for 3 consecutive days without platelet transfusion in the peripheral blood of the recipient. In some embodiments, a population of subjects administered a composition of the disclosure achieve platelet engraftment by a median of approximately 7 days, about 8 days, about 9 days, about 10 days, about 11 days, about 12 days, about 12 days, about 14 days, or about 15 days post-transplant. In some embodiments, a population of subjects administered a composition of the disclosure achieve platelet engraftment by a median of 13 days post-transplant. In some embodiments, a population of subjects administered a composition of the disclosure achieve platelet engraftment by a median of 12 days post-transplant. In some embodiments, a population of subjects administered a composition of the disclosure achieve platelet engraftment by a median of 11 days post-transplant.

In some embodiments, subjects administered a composition of the disclosure (e.g., a cell population as described herein) have increased incidence, timing, or both incidence and timing of platelet engraftment, relative to a corresponding human subject that has been administered a standard myeloablative allogeneic hematopoietic stem cell transplant (alloHSCT). In some embodiments, platelet engraftment occurs approximately 1 day or more, approximately 2 days or more, approximately 3 days or more, approximately 4 days or more, approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 21 days or more, approximately 22 days or more, approximately 23 days or more, approximately 24 days or more, approximately 25 days or more, approximately 26 days or more, approximately 27 days or more, approximately 28 days or more, approximately 29 days or more, approximately 30 days or more, approximately 31 days or more, approximately 32 days or more, approximately 33 days or more, approximately 34 days or more, approximately 35 days or more, approximately 36 days or more, approximately 37 days or more, approximately 38 days or more, approximately 39 days or more, approximately 40 days or more, approximately 41 days or more, approximately 42 days or more, approximately 43 days or more, approximately 44 days or more, approximately 45 days or more, approximately 46 days or more, approximately 47 days or more, approximately 48 days or more, approximately 49 days or more, approximately 50 days or more, approximately 51 days or more, approximately 52 days or more, approximately 53 days or more, approximately 54 days or more, approximately 55 days or more, approximately 56 days or more, approximately 57 days or more, approximately 58 days or more, approximately 59 days or more, approximately 60 days or more, approximately 61 days or more, approximately 62 days or more, approximately 63 days or more, approximately 64 days or more, approximately 65 days or more, approximately 66 days or more, approximately 67 days or more, approximately 68 days or more, approximately 69 days or more, approximately 70 days or more, approximately 71 days or more, approximately 72 days or more, approximately 73 days or more, approximately 74 days or more, approximately 75 days or more, approximately 76 days or more, approximately 77 days or more, approximately 78 days or more, approximately 79 days or more, approximately 80 days or more, approximately 81 days or more, approximately 82 days or more, approximately 83 days or more, approximately 84 days or more, approximately 85 days or more, approximately 86 days or more, approximately 87 days or more, approximately 88 days or more, approximately 89 days or more, or 90 days or more after administration of the composition of the disclosure.

### T Cell Engraftment

A population of subjects that are administered a composition of the disclosure (e.g., a cell population as described herein) exhibit a high proportion of circulating Tregs, for example, a higher proportion of circulating Tregs compared to subjects that are administered an alternate composition. In some embodiments, an average of at least about 5%, at least about 7.5%, at least about 10%, at least about 12.5%, at least about 15%, at least about 16%, at least about 17%, at least about 18%, at least about 19%, at least about 20%, at least about 21%, at least about 22%, at least about 23%, at least about 24%, or at least about 25% of circulating CD4⁺ T cells are Tregs when subjects are evaluated a suitable amount of time post-transplant, for example, at approximately 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, or 180 days post-transplant. In some embodiments, an average of at least about 15% of circulating CD4⁺ T cells are Tregs when subjects are evaluated 28 days post-transplant.

A population of subjects that are administered a composition of the disclosure (e.g., a cell population as described herein) exhibit a normal CD4:CD8 T cell ratio, for example, a higher CD4:CD8 T cell ratio compared to subjects that are administered an alternate composition or normal healthy control subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥0.8 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant. In some embodiments, at least 50% of subjects have a CD4:CD8 T cell ratio of ≥1 when evaluated 28 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.2 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

A population of subjects that are administered a composition of the disclosure (e.g., a cell population as described herein) exhibit a high proportion of donor-derived circulating T cells at an early timepoint after transplant, for example, a higher proportion of donor-derived circulating T cells compared to subjects that are administered an alternate composition.

In some embodiments, more than 50% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 60% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 70% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 80% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 90% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 50% of circulating T cells are donor-derived in at least about at least about 70% of subjects evaluated 30 days post-transplant. In some embodiments, more than 70% of circulating T cells are donor-derived in at least about at least about 60% of subjects evaluated 30 days post-transplant.

### B Cell Engraftment

A population of subjects that are administered a composition of the disclosure (e.g., a cell population as described herein) exhibit a high concentration of circulating B cells at an early timepoint after transplant, for example, a higher concentration of circulating B cells compared to subjects that are administered an alternate composition. Achieving a high concentration of circulating B cells at an early timepoint after transplant can be important for immunocompetence, and can allow vaccines to elicit protective immune responses at an earlier timepoint post-transplant.

In some embodiments, more than about 50 B cells per uL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 50 B cells per μL are present in the blood of at least 75% of subjects evaluated about 60 days post-transplant.

In some embodiments, more than about 60 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 60 B cells per μL are present in the blood of at least 75% of subjects evaluated about 60 days post-transplant.

In some embodiments, more than about 70 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 70 B cells per μL are present in the blood of at least 75% of subjects evaluated about 60 days post-transplant.

In some embodiments, more than about 80 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, more than about 90 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 90 B cells per μL are present in the blood of at least 75% of subjects evaluated about 180 days post-transplant.

In some embodiments, more than about 100 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 100 B cells per μL are present in the blood of at least 75% of subjects evaluated about 180 days post-transplant.

In some embodiments, more than about 110 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 110 B cells per μL are present in the blood of at least 75% of subjects evaluated about 180 days post-transplant.

A population of subjects that are administered a composition of the disclosure (e.g., a cell population as described herein) exhibit a high proportion of mature B cells at an early timepoint after transplant, for example, a higher proportion of circulating B cells that are mature B cells (e.g., IgD⁺ and/or CD27⁺) compared to subjects that are administered an alternate composition. Achieving a high proportion of mature B cells at an early timepoint after transplant can be important for immunocompetence, and can allow vaccines to elicit protective immune responses at an earlier timepoint post-transplant.

In some embodiments, at least about 50% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 60% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 70% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 80% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 80% of circulating CD19⁺ B cells are IgD⁺ in at least about 75% of subjects evaluated about 100 days post-transplant.

In some embodiments, at least about 90% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 90% of circulating CD19⁺ B cells are IgD⁺ in at least about 75% of subjects evaluated about 100 days post-transplant.

In some embodiments, at least about 25% of circulating CD19⁺ B cells are CD27⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 30% of circulating CD19⁺ B cells are CD27⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 30% of circulating CD19⁺ B cells are CD27⁺ in at least about 75% of subjects evaluated at 100 days post-transplant.

In some embodiments, at least about 35% of circulating CD19⁺ B cells are CD27⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 35% of circulating CD19⁺ B cells are CD27⁺ in at least about 75% of subjects evaluated at 100 days post-transplant.

In some embodiments, at least about 40% of circulating CD19⁺ B cells are CD27⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 40% of circulating CD19⁺ B cells are CD27⁺ in at least about 75% of subjects evaluated at 100 days post-transplant.

Clinical outcomes disclosed herein can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

### Kits

Another aspect provides a kit that comprises a solution comprising a first container comprising a first population of CD45⁺ cells, a second container comprising a solution comprising a third population of CD45⁺ cells, and a third container comprising a solution comprising a population of cells enriched for regulatory T cells (Tregs). In this aspect, the solution comprising the first population of CD45⁺ cells, the solution comprising the third population of CD45⁺ cells, and the solution comprising the population of cells enriched for Tregs are as defined according to any herein disclosed multi-component pharmaceutical treatment or method. In various embodiments, the kit further comprises a fourth container comprising the GVHD prophylactic agent. In some cases, the further comprising instructions for performing any herein-disclosed method.

A further aspect provides a kit comprising: (a) one or more reagents to sort CD34⁺ cells from a mobilized peripheral blood composition; (b) one or more reagents to sort regulatory T cells (Tregs) from the mobilized peripheral blood composition; (c) one or more reagents to detect a number of CD3⁺ conventional T cells in the mobilized peripheral blood; and (d) a solution comprising one or more doses of a graft vs host disease (GVHD) prophylactic agent. In some embodiments, the kit further comprises instructions for performing any herein-disclosed method.

Various embodiments provide a kit comprising one or more reagents for sorting HSPCs, for instance, a kit may comprise reagents to enrich a CD34⁺ cell population from a mobilized peripheral blood donation.

Some embodiments provide a kit comprising one or more reagents for sorting Tregs, for instance, a kit may comprise reagents to enrich a Treg cell population (using markers as described elsewhere herein) from a mobilized peripheral blood donation.

Embodiments provides a kit comprising one or more conditioning reagents for a conditioning regimen, for instance, a kit may comprise reagents for myeloablation or myeloreduction of a recipient.

Some embodiments provide a kit comprising one or more GVHD prophylactic agents.

In any herein-disclosed method, the method further comprises providing instructions for use (IFU), the IFU including instructions for administering the cell populations to the patient. In some cases, the IFU also include instructions for administering one or more pharmaceutical agents or compositions to the patient.

### Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present Specification and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

By “one or more” or “at least one” is meant at least one, e.g., one, two, three, four, five, six, seven, eight, nine, ten or more.

The term “similar” is understood to be resembling up to and including identical. Therefore two (or more) items may be identical, substantially identical, comprise equivalent components, comprise substitutable components, comprise analogous components, comprise comparable components, comprise complementary components, comprise related components, comprise like components, and/or the differences between the two (or more items) are insubstantial and/or result in a composition having equivalent properties, identical properties, substantially identical properties, and the like.

The term “treatment” (and grammatical variations thereof such as “treat” or “treating”) can refer to complete or partial amelioration or reduction of a disease or condition or disorder, or a symptom, adverse effect or outcome, or phenotype associated therewith. Desirable effects of treatment may include, without limitation, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

The term “preventing,” includes providing prophylaxis with respect to the occurrence or recurrence of a disease in a subject that may be predisposed to the disease but has not yet been diagnosed with the disease. In some embodiments, the provided compositions, treatments, and therapies are used to delay development of a disease or to slow the progression of a disease. The terms “patient” and “subject” are synonyms.

Any aspect or embodiment described herein can be combined with any other aspect or embodiment as disclosed herein.

### ADDITIONAL ASPECTS

Aspects provide a method of transplanting a conventional T cell (Tcon) population into a human subject without eliciting a stage 2 or higher graft versus host disease (GVHD) response up to approximately 100 days after transplanting. The method comprises (i) administering a heterogenous cell population comprising lymphocytes, granulocytes, and monocytes, wherein at least about 30% of the lymphocytes comprise conventional T cells (Tcons); and (ii). administering a population of regulatory T cells (Tregs). In this method, the heterogenous cell component and/or the population of Tregs comprise less than about 5 EU/ml endotoxins.

A further aspect provides a method of treating a human subject comprising a step (a) of administering a plurality of populations of cells, in which the plurality of populations of cells comprises: (i). a population of hematopoietic stem and progenitor cells (HSPCs); (ii) a population of cells comprising regulatory T cells (Tregs); and (iii) a population of conventional T cells (Tcons); and a step (b) of administering no more than one graft versus host disease (GVHD) prophylactic agent for less than about 120 days. In this method, the population of HSPCs comprises less than about 2% CD3⁺ cells.

An aspects is a method treating a human subject in need thereof comprising administering to the human subject at least two pharmaceutical compositions, wherein the pharmaceutical compositions are selected from (a) a pharmaceutical composition comprising a population of hematopoietic stem and progenitor cells (HSPCs); (b) a pharmaceutical composition comprising a population of regulatory T cells (Tregs); and (c) a pharmaceutical composition comprising a population of conventional T cells (Tcons). In this method, the pharmaceutical compositions (a), (b) and (c) comprise less than about 5 EU/ml endotoxins each; and less than 15 human subjects in a group of 100 human subjects administered the two or more pharmaceutical compositions develops a stage 2 or higher graft versus host disease (GVHD) response within about 30 days after being administered the pharmaceutical composition comprising the population of Tcons.

An additional aspect is a method of transplanting a conventional T cell (Tcon) population into a human subject without eliciting a stage 2 or higher graft versus host disease (GVHD) response up to approximately 100 days after transplanting. The method comprising: (i). administering a solution comprising a population of conventional T cells (Tcons); and (ii). administering a solution comprising a population of regulatory T cells (Tregs). In this method, the population of Tcons is cryopreserved for at least about 4 hours; and the solution comprising the population of Tcons and the solution comprising the population of Tregs comprise less than about 5 EU of endotoxins per ml of the solution.

A yet another aspect provides a method of treating a hematologic malignancy in a human subject in need thereof, the method comprising administering to the human subject: (a) a population of hematopoietic stem and progenitor cells (HSPCs); (b) a population of regulatory T cells (Tregs); and (c) a population of conventional T cells (Tcons). In the method, the population of HSPCs and the population of Tregs are administered prior to the population of Tcons; and peripheral blood of the human subject exhibits an elevated Treg count until about 100 days after the administering the three populations of cells as compared to a healthy human subject that was not administered the three populations of cells.

A further aspect provides a method of transplanting a conventional T cell (Tcon) population into a human subject without eliciting a stage 2 or higher graft versus host disease (GVHD) response up to approximately 100 days after transplanting. The method comprising (i). administering a population of conventional T cells (Tcons); and (ii). administering a population of regulatory T cells (Tregs). In this method, the population of Tcons is administered at least about 12 hours after the population of Tregs is administered; and the population of Tcons and the population of Tregs comprise less than about 5 EU/ml endotoxins.

### ENUMERATED EMBODIMENTS

The following enumerated embodiments describe further non-limiting aspects of the present disclosure.
Embodiment 1. A multi-component cellular therapy product comprising:
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, wherein the first population of CD45⁺ cells is formulated with an excipient at a neutral pH;
b) a second single dose transfer bag comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the product, wherein the second population of CD45⁺ cells is formulated with an excipient at a neutral pH; and
c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, wherein the third population of CD45⁺ cells is formulated with an excipient at a neutral pH, wherein the excipient comprises one more cryoprotectants; or
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, and comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the product, wherein the first population of CD45⁺ cells and the second population of CD45⁺ cells are formulated with an excipient at a neutral pH; and
b) a second single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, wherein the third population of CD45⁺ cells is formulated with an excipient at a neutral pH, wherein the excipient comprises one more cryoprotectants.
Embodiment 2. The multi-component cellular therapy product of embodiment 1, wherein the first population of isolated CD45⁺ cells comprises a dose of approximately 1.0×10⁵ or more HSPCs per kilogram of body weight of the human subject receiving the product, approximately 2.0×10⁵ or more HSPCs per kilogram of body weight of the human subject receiving the product, approximately 3.0×10⁵ or more HSPCs per kilogram of body weight of the human subject receiving the product, approximately 4.0×10⁵ or more HSPCs per kilogram of body weight of the human subject receiving the product, approximately 5.0×10⁵ or more HSPCs per kilogram of body weight of the human subject receiving the product, approximately 6.0×10⁵ or more HSPCs per kilogram of body weight of the human subject receiving the product, approximately 7.0×10⁵ or more HSPCs per kilogram of body weight of the human subject receiving the product, approximately 8.0×10⁵ or more HSPCs per kilogram of body weight of the human subject receiving the product, approximately 9.0×10⁵ or more HSPCs per kilogram of body weight of the human subject receiving the product, approximately 1.0×10⁶ or more HSPCs per kilogram of body weight of the human subject receiving the product, approximately 1.5×10⁶ or more HSPCs per kilogram of body weight of the human subject receiving the product, approximately 2.0×10⁶ or more HSPCs per kilogram of body weight of the human subject receiving the product, approximately 2.5×10⁶ or more HSPCs per kilogram of body weight of the human subject receiving the product, approximately 3.0×10⁶ or more HSPCs per kilogram of body weight of the human subject receiving the product, approximately 3.5×10⁶ or more HSPCs per kilogram of body weight of the human subject receiving the product, approximately 4.0×10⁶ or more HSPCs per kilogram of body weight of the human subject receiving the product, approximately 4.5×10⁶ or more HSPCs per kilogram of body weight of the human subject receiving the product, approximately 5.0×10⁶ or more HSPCs per kilogram of body weight of the human subject receiving the product, approximately 5.5×10⁶ or more HSPCs per kilogram of body weight of the human subject receiving the product, approximately 6.0×10⁶ or more HSPCs per kilogram of body weight of the human subject receiving the product, approximately 6.5×10⁶ or more HSPCs per kilogram of body weight of the human subject receiving the product, approximately 7.0×10⁶ or more HSPCs per kilogram of body weight of the human subject receiving the product, approximately 7.5×10⁶ or more HSPCs per kilogram of body weight of the human subject receiving the product, approximately 8.0×10⁶ or more HSPCs per kilogram of body weight of the human subject receiving the product, approximately 8.5×10⁶ or more HSPCs per kilogram of body weight of the human subject receiving the product, approximately 9.0×10⁶ or more HSPCs per kilogram of body weight of the human subject receiving the product, approximately 9.5×10⁶ or more HSPCs per kilogram of body weight of the human subject receiving the product, approximately 1.0×10⁷ or more HSPCs per kilogram of body weight of the human subject receiving the product, approximately 1.5×10⁷ or more HSPCs per kilogram of body weight of the human subject receiving the product, approximately 2.0×10⁷ or more HSPCs per kilogram of body weight of the human subject receiving the product, approximately 2.5×10⁷ or more HSPCs per kilogram of body weight of the human subject receiving the product, approximately 3.0×10⁷ or more HSPCs per kilogram of body weight of the human subject receiving the product, approximately 3.5×10⁷ or more HSPCs per kilogram of body weight of the human subject receiving the product, approximately 4.0×10⁷ or more HSPCs per kilogram of body weight of the human subject receiving the product, approximately 4.5×10⁷ or more HSPCs per kilogram of body weight of the human subject receiving the product, approximately 5.0×10⁷ or more HSPCs per kilogram of body weight of the human subject receiving the product, approximately 5.5×10⁷ or more HSPCs per kilogram of body weight of the human subject receiving the product, approximately 6.0×10⁷ or more HSPCs per kilogram of body weight of the human subject receiving the product, approximately 6.5×10⁷ or more HSPCs per kilogram of body weight of the human subject receiving the product, approximately 7.0×10⁷ or more HSPCs per kilogram of body weight of the human subject receiving the product, approximately 7.5×10⁷ or more HSPCs per kilogram of body weight of the human subject receiving the product, approximately 8.0×10⁷ or more HSPCs per kilogram of body weight of the human subject receiving the product, approximately 8.5×10⁷ or more HSPCs per kilogram of body weight of the human subject receiving the product, approximately 9.0×10⁷ or more HSPCs per kilogram of body weight of the human subject receiving the product, approximately 9.5×10⁷ or more HSPCs per kilogram of body weight of the human subject receiving the product, or approximately 1.0×10⁸ or more HSPCs per kilogram of body weight of the human subject receiving the product.
Embodiment 3. The multi-component cellular therapy product of embodiment 1 or embodiment 2, wherein the second population of isolated CD45⁺ cells comprises a dose of approximately 1.0×10⁵ or more isolated fresh Tregs per kilogram of body weight of the human subject receiving the product, approximately 2.0×10⁵ or more isolated fresh Tregs per kilogram of body weight of the human subject receiving the product, approximately 3.0×10⁵ or more isolated fresh Tregs per kilogram of body weight of the human subject receiving the product, approximately 4.0×10⁵ or more isolated fresh Tregs per kilogram of body weight of the human subject receiving the product, approximately 5.0×10⁵ or more isolated fresh Tregs per kilogram of body weight of the human subject receiving the product, approximately 6.0×10⁵ or more isolated fresh Tregs per kilogram of body weight of the human subject receiving the product, approximately 7.0×10⁵ or more isolated fresh Tregs per kilogram of body weight of the human subject receiving the product, approximately 8.0×10⁵ or more isolated fresh Tregs per kilogram of body weight of the human subject receiving the product, approximately 9.0×10⁵ or more isolated fresh Tregs per kilogram of body weight of the human subject receiving the product, approximately 1.0×10⁶ or more isolated fresh Tregs per kilogram of body weight of the human subject receiving the product, approximately 1.5×10⁶ or more isolated fresh Tregs per kilogram of body weight of the human subject receiving the product, approximately 2.0×10⁶ or more isolated fresh Tregs per kilogram of body weight of the human subject receiving the product, approximately 2.5×10⁶ or more isolated fresh Tregs per kilogram of body weight of the human subject receiving the product, approximately 3.0×10⁶ or more isolated fresh Tregs per kilogram of body weight of the human subject receiving the product, approximately 3.5×10⁶ or more isolated fresh Tregs per kilogram of body weight of the human subject receiving the product, approximately 4.0×10⁶ or more isolated fresh Tregs per kilogram of body weight of the human subject receiving the product, approximately 4.5×10⁶ or more isolated fresh Tregs per kilogram of body weight of the human subject receiving the product, approximately 5.0×10⁶ or more isolated fresh Tregs per kilogram of body weight of the human subject receiving the product, approximately 5.5×10⁶ or more isolated fresh Tregs per kilogram of body weight of the human subject receiving the product, approximately 6.0×10⁶ or more isolated fresh Tregs per kilogram of body weight of the human subject receiving the product, approximately 6.5×10⁶ or more isolated fresh Tregs per kilogram of body weight of the human subject receiving the product, approximately 7.0×10⁶ or more isolated fresh Tregs per kilogram of body weight of the human subject receiving the product, approximately 7.5×10⁶ or more isolated fresh Tregs per kilogram of body weight of the human subject receiving the product, approximately 8.0×10⁶ or more isolated fresh Tregs per kilogram of body weight of the human subject receiving the product, approximately 8.5×10⁶ or more isolated fresh Tregs per kilogram of body weight of the human subject receiving the product, approximately 9.0×10⁶ or more isolated fresh Tregs per kilogram of body weight of the human subject receiving the product, approximately 9.5×10⁶ or more isolated fresh Tregs per kilogram of body weight of the human subject receiving the product, approximately 1.0×10⁷ or more isolated fresh Tregs per kilogram of body weight of the human subject receiving the product, approximately 1.5×10⁷ or more isolated fresh Tregs per kilogram of body weight of the human subject receiving the product, or approximately 2.0×10⁷ or more isolated fresh Tregs per kilogram of body weight of the human subject receiving the product.
Embodiment 4. The multi-component cellular therapy product of any one of embodiments 1-3, wherein the third population of isolated CD45⁺ cells comprises a dose of approximately 1.0×10⁵ or more Tcons per kilogram of body weight of the human subject receiving the product, approximately 2.0×10⁵ or more Tcons per kilogram of body weight of the human subject receiving the product, approximately 3.0×10⁵ or more Tcons per kilogram of body weight of the human subject receiving the product, approximately 4.0×10⁵ or more Tcons per kilogram of body weight of the human subject receiving the product, approximately 5.0×10⁵ or more Tcons per kilogram of body weight of the human subject receiving the product, approximately 6.0×10⁵ or more Tcons per kilogram of body weight of the human subject receiving the product, approximately 7.0×10⁵ or more Tcons per kilogram of body weight of the human subject receiving the product, approximately 8.0×10⁵ or more Tcons per kilogram of body weight of the human subject receiving the product, approximately 9.0×10⁵ or more Tcons per kilogram of body weight of the human subject receiving the product, approximately 1.0×10⁶ or more Tcons per kilogram of body weight of the human subject receiving the product, approximately 1.5×10⁶ or more Tcons per kilogram of body weight of the human subject receiving the product, approximately 2.0×10⁶ or more Tcons per kilogram of body weight of the human subject receiving the product, approximately 2.5×10⁶ or more Tcons per kilogram of body weight of the human subject receiving the product, approximately 3.0×10⁶ or more Tcons per kilogram of body weight of the human subject receiving the product, approximately 3.5×10⁶ or more Tcons per kilogram of body weight of the human subject receiving the product, approximately 4.0×10⁶ or more Tcons per kilogram of body weight of the human subject receiving the product, approximately 4.5×10⁶ or more Tcons per kilogram of body weight of the human subject receiving the product, approximately 5.0×10⁶ or more Tcons per kilogram of body weight of the human subject receiving the product, approximately 5.5×10⁶ or more Tcons per kilogram of body weight of the human subject receiving the product, approximately 6.0×10⁶ or more Tcons per kilogram of body weight of the human subject receiving the product, approximately 6.5×10⁶ or more Tcons per kilogram of body weight of the human subject receiving the product, approximately 7.0×10⁶ or more Tcons per kilogram of body weight of the human subject receiving the product, approximately 7.5×10⁶ or more Tcons per kilogram of body weight of the human subject receiving the product, approximately 8.0×10⁶ or more Tcons per kilogram of body weight of the human subject receiving the product, approximately 8.5×10⁶ or more Tcons per kilogram of body weight of the human subject receiving the product, approximately 9.0×10⁶ or Tcons per kilogram of body weight of the human subject receiving the product, approximately 9.5×10⁶ or more Tcons per kilogram of body weight of the human subject receiving the product, approximately 1.0×10⁷ or more Tcons per kilogram of body weight of the human subject receiving the product, approximately 1.5×10⁷ or more Tcons per kilogram of body weight of the human subject receiving the product, approximately 2.0×10⁷ or more Tcons per kilogram of body weight of the human subject receiving the product, approximately 2.5×10⁷ or more Tcons per kilogram of body weight of the human subject receiving the product, approximately 3.0×10⁷ or more Tcons per kilogram of body weight of the human subject receiving the product, approximately 3.5×10⁷ or more Tcons per kilogram of body weight of the human subject receiving the product, or approximately 4.0×10⁷ or more Tcons per kilogram of body weight of the human subject receiving the product.
Embodiment 5. A multi-component cellular therapy product comprising:
a) a first single dose transfer bag comprising a first population of isolated CD45⁺ cells comprising a dose of approximately 1.5×10⁷ to approximately 1.5×10¹⁰ CD34⁺ hematopoietic stem and progenitor cells (HSPCs), wherein the first population of CD45⁺ cells is formulated with an excipient at a neutral pH;
b) a second single dose transfer bag comprising a second population of isolated CD45⁺ cells comprising a dose of approximately 1.5×10⁷ to approximately 3.0×10⁹ isolated fresh CD4⁺CD25⁺CD127^{dim} regulatory T cells (Tregs), wherein the second population of isolated CD45⁺ cells is formulated with an excipient at a neutral pH; and
c) a third single dose transfer bag comprising a third population of isolated CD45⁺ cells comprising a dose of approximately 1.5×10⁷ to approximately 6.0×10⁹ conventional CD3⁺ T cells (Tcons), wherein the third population of isolated CD45⁺ cells is formulated with an excipient at a neutral pH, wherein the excipient comprises one more cryoprotectants; or
a) a first single dose transfer bag comprising a first population of isolated CD45⁺ cells comprising a dose of approximately 1.5×10⁷ to approximately 1.5×10¹⁰ CD34⁺ hematopoietic stem and progenitor cells (HSPCs), and comprising a second population of isolated CD45⁺ cells comprising a dose of approximately 1.5×10⁷ to approximately 3.0×10⁹ isolated fresh CD4⁺ CD25⁺ CD127^{dim} regulatory T cells (Tregs), wherein the first population of CD45⁺ cells and the second population of isolated CD45⁺ cells are formulated with an excipient at a neutral pH; and
b) a second single dose transfer bag comprising a third population of isolated CD45⁺ cells comprising a dose of approximately 1.5×10⁷ to approximately 6.0×10⁹ conventional CD3⁺ T cells (Tcons), wherein the third population of isolated CD45⁺ cells is formulated with an excipient at a neutral pH, wherein the excipient comprises one more cryoprotectants.
Embodiment 6. A multi-component cellular therapy product comprising:
a) a first single dose transfer bag comprising a first population of isolated CD45⁺ cells comprising a dose of approximately 5.0×10⁵ to approximately 5.0×10⁸ CD34⁺ hematopoietic stem and progenitor cells (HSPCs), wherein the first population of CD45⁺ cells is formulated with an excipient at a neutral pH;
b) a second single dose transfer bag comprising a second population of isolated CD45⁺ cells comprising a dose of approximately 5.0×10⁵ to approximately 1.0×10⁸ isolated fresh CD4⁺CD25⁺CD127^{dim} regulatory T cells (Tregs), wherein the second population of isolated CD45⁺ cells is formulated with an excipient at a neutral pH; and
c) a third single dose transfer bag comprising a third population of isolated CD45⁺ cells comprising a dose of approximately 5.0×10⁵ to approximately 2.0×10⁸ conventional CD3⁺ T cells (Tcons), wherein the third population of isolated CD45⁺ cells is formulated with an excipient at a neutral pH, wherein the excipient comprises one more cryoprotectants; or
a) a first single dose transfer bag comprising a first population of isolated CD45⁺ cells comprising a dose of approximately 5.0×10⁵ to approximately 5.0×10⁸ CD34⁺ hematopoietic stem and progenitor cells (HSPCs), and comprising a second population of isolated CD45⁺ cells comprising a dose of approximately 5.0×10⁵ to approximately 1.0×10⁸ isolated fresh CD4⁺ CD25⁺ CD127^{dim} regulatory T cells (Tregs), wherein the first population of isolated CD45⁺ cells and the second population of isolated CD45⁺ cells are formulated with an excipient at a neutral pH; and
b) a second single dose transfer bag comprising a third population of isolated CD45⁺ cells comprising a dose of approximately 5.0×10⁵ to approximately 2.0×10⁸ conventional CD3⁺ T cells (Tcons), wherein the third population of isolated CD45⁺ cells is formulated with an excipient at a neutral pH, wherein the excipient comprises one more cryoprotectants.
Embodiment 7. The multi-component cellular therapy product of embodiment 5 or embodiment 6, wherein the first population of isolated CD45⁺ cells comprises a dose of approximately 5.0×10⁵ or more HSPCs, approximately 6.0×10⁵ or more HSPCs, approximately 7.0×10⁵ or more HSPCs, approximately 8.0×10⁵ or more HSPCs, approximately 9.0×10⁵ or more HSPCs, approximately 1.0×10⁶ or more HSPCs, approximately 1.5×10⁶ or more HSPCs, approximately 2.0×10⁶ or more HSPCs, approximately 2.5×10⁶ or more HSPCs, approximately 3.0×10⁶ or more HSPCs, approximately 3.5×10⁶ or more HSPCs, approximately 4.0×10⁶ or more HSPCs, approximately 4.5×10⁶ or more HSPCs, approximately 5.0×10⁶ or more HSPCs, approximately 5.5×10⁶ or more HSPCs, approximately 6.0×10⁶ or more HSPCs, approximately 6.5×10⁶ or more HSPCs, approximately 7.0×10⁶ or more HSPCs, approximately 7.5×10⁶ or more HSPCs, approximately 8.0×10⁶ or more HSPCs, approximately 8.5×10⁶ or more HSPCs, approximately 9.0×10⁶ or more HSPCs, approximately 9.5×10⁶ or more HSPCs, approximately 1.0×10⁷ or more HSPCs, approximately 1.5×10⁷ or more HSPCs, approximately 2.0×10⁷ or more HSPCs, approximately 2.5×10⁷ or more HSPCs, approximately 3.0×10⁷ or more HSPCs, approximately 3.5×10⁷ or more HSPCs, approximately 4.0×10⁷ or more HSPCs, approximately 4.5×10⁷ or more HSPCs, approximately 5.0×10⁷ or more HSPCs, approximately 5.5×10⁷ or more HSPCs, approximately 6.0×10⁷ or more HSPCs, approximately 6.5×10⁷ or more HSPCs, approximately 7.0×10⁷ or more HSPCs, approximately 7.5×10⁷ or more HSPCs, approximately 8.0×10⁷ or more HSPCs, approximately 8.5×10⁷ or more HSPCs, approximately 9.0×10⁷ or more HSPCs, approximately 9.5×10⁷ or more HSPCs, approximately 1.0×10⁸ or more HSPCs, approximately 1.5×10⁸ or more HSPCs, approximately 2.0×10⁸ or more HSPCs, approximately 2.5×10⁸ or more HSPCs, approximately 3.0×10⁸ or more HSPCs, approximately 3.5×10⁸ or more HSPCs, approximately 4.0×10⁸ or more HSPCs, approximately 4.5×10⁸ or more HSPCs, approximately 5.0×10⁸ or more HSPCs, approximately 5.5×10⁸ or more HSPCs, approximately 6.0×10⁸ or more HSPCs, approximately 6.5×10⁸ or more HSPCs, approximately 7.0×10⁸ or more HSPCs, approximately 7.5×10⁸ or more HSPCs, approximately 8.0×10⁸ or more HSPCs, approximately 8.5×10⁸ or more HSPCs, approximately 9.0×10⁸ or more HSPCs, approximately 9.5×10⁸ or more HSPCs, approximately 1.0×10⁹ or more HSPCs, approximately 1.5×10⁹ or more HSPCs, approximately 2.0×10⁹ or more HSPCs, approximately 2.5×10⁹ or more HSPCs, approximately 3.0×10⁹ or more HSPCs, approximately 3.5×10⁹ or more HSPCs, approximately 4.0×10⁹ or more HSPCs, approximately 4.5×10⁹ or more HSPCs, approximately 5.0×10⁹ or more HSPCs, approximately 5.5×10⁹ or more HSPCs, approximately 6.0×10⁹ or more HSPCs, approximately 6.5×10⁹ or more HSPCs, approximately 7.0×10⁹ or more HSPCs, approximately 7.5×10⁹ or more HSPCs, approximately 8.0×10⁹ or more HSPCs, approximately 8.5×10⁹ or more HSPCs, approximately 9.0×10⁹ or more HSPCs, approximately 9.5×10⁹ or more HSPCs, approximately 1.0×10¹⁰ or more HSPCs, or approximately 1.5×10¹⁰ or more HSPCs.
Embodiment 8. The multi-component cellular therapy product of any one of embodiments 5-7, wherein the second population of isolated CD45⁺ cells comprises a dose of approximately 5.0×10⁵ or more isolated fresh Tregs, approximately 6.0×10⁵ or more isolated fresh Tregs, approximately 7.0×10⁵ or more isolated fresh Tregs, approximately 8.0×10⁵ or more isolated fresh Tregs, approximately 9.0×10⁵ or more isolated fresh Tregs, approximately 1.0×10⁶ or more isolated fresh Tregs, approximately 1.5×10⁶ or more isolated fresh Tregs, approximately 2.0×10⁶ or more isolated fresh Tregs, approximately 2.5×10⁶ or more isolated fresh Tregs, approximately 3.0×10⁶ or more isolated fresh Tregs, approximately 3.5×10⁶ or more isolated fresh Tregs, approximately 4.0×10⁶ or more isolated fresh Tregs, approximately 4.5×10⁶ or more isolated fresh Tregs, approximately 5.0×10⁶ or more isolated fresh Tregs, approximately 5.5×10⁶ or more isolated fresh Tregs, approximately 6.0×10⁶ or more isolated fresh Tregs, approximately 6.5×10⁶ or more isolated fresh Tregs, approximately 7.0×10⁶ or more isolated fresh Tregs, approximately 7.5×10⁶ or more isolated fresh Tregs, approximately 8.0×10⁶ or more isolated fresh Tregs, approximately 8.5×10⁶ or more isolated fresh Tregs, approximately 9.0×10⁶ or more isolated fresh Tregs, approximately 9.5×10⁶ or more isolated fresh Tregs, approximately 1.0×10⁷ or more isolated fresh Tregs, approximately 1.5×10⁷ or more isolated fresh Tregs, approximately 2.0×10⁷ or more isolated fresh Tregs, approximately 2.5×10⁷ or more isolated fresh Tregs, approximately 3.0×10⁷ or more isolated fresh Tregs, approximately 3.5×10⁷ or more isolated fresh Tregs, approximately 4.0×10⁷ or more isolated fresh Tregs, approximately 4.5×10⁷ or more isolated fresh Tregs, approximately 5.0×10⁷ or more isolated fresh Tregs, approximately 5.5×10⁷ or more isolated fresh Tregs, approximately 6.0×10⁷ or more isolated fresh Tregs, approximately 6.5×10⁷ or more isolated fresh Tregs, approximately 7.0×10⁷ or more isolated fresh Tregs, approximately 7.5×10⁷ or more isolated fresh Tregs, approximately 8.0×10⁷ or more isolated fresh Tregs, approximately 8.5×10⁷ or more isolated fresh Tregs, approximately 9.0×10⁷ or more isolated fresh Tregs, approximately 9.5×10⁷ or more isolated fresh Tregs, approximately 1.0×10⁸ or more isolated fresh Tregs, approximately 1.5×10⁸ or more isolated fresh Tregs, approximately 2.0×10⁸ or more isolated fresh Tregs, approximately 2.5×10⁸ or more isolated fresh Tregs, approximately 3.0×10⁸ or more isolated fresh Tregs, approximately 3.5×10⁸ or more isolated fresh Tregs, approximately 4.0×10⁸ or more isolated fresh Tregs, approximately 4.5×10⁸ or more isolated fresh Tregs, approximately 5.0×10⁸ or more isolated fresh Tregs, approximately 5.5×10⁸ or more isolated fresh Tregs, approximately 6.0×10⁸ or more isolated fresh Tregs, approximately 6.5×10⁸ or more isolated fresh Tregs, approximately 7.0×10⁸ or more isolated fresh Tregs, approximately 7.5×10⁸ or more isolated fresh Tregs, approximately 8.0×10⁸ or more isolated fresh Tregs, approximately 8.5×10⁸ or more isolated fresh Tregs, approximately 9.0×10⁸ or more isolated fresh Tregs, approximately 9.5×10⁸ or more isolated fresh Tregs, approximately 1.0×10⁹ or more isolated fresh Tregs, approximately 1.5×10⁹ or more isolated fresh Tregs, approximately 2.0×10⁹ or more isolated fresh Tregs, approximately 2.5×10⁹ or more isolated fresh Tregs, or approximately 3.0×10⁹ or more isolated fresh Tregs.
Embodiment 9. The multi-component cellular therapy product of any one of embodiments 5-8, wherein the third population of isolated CD45⁺ cells comprises a dose of approximately 5.0×10⁵ or more Tcons, approximately 6.0×10⁵ or more Tcons, approximately 7.0×10⁵ or more Tcons, approximately 8.0×10⁵ or more Tcons, approximately 9.0×10⁵ or more Tcons, approximately 1.0×10⁶ or more Tcons, approximately 1.5×10⁶ or more Tcons, approximately 2.0×10⁶ or more Tcons, approximately 2.5×10⁶ or more Tcons, approximately 3.0×10⁶ or more Tcons, approximately 3.5×10⁶ or more Tcons, approximately 4.0×10⁶ or more Tcons, approximately 4.5×10⁶ or more Tcons, approximately 5.0×10⁶ or more Tcons, approximately 5.5×10⁶ or more Tcons, approximately 6.0×10⁶ or more Tcons, approximately 6.5×10⁶ or more Tcons, approximately 7.0×10⁶ or more Tcons, approximately 7.5×10⁶ or more Tcons, approximately 8.0×10⁶ or more Tcons, approximately 8.5×10⁶ or more Tcons, approximately 9.0×10⁶ or Tcons, approximately 9.5×10⁶ or more Tcons, approximately 1.0×10⁷ or more Tcons, approximately 1.5×10⁷ or more Tcons, approximately 2.0×10⁷ or more Tcons, approximately 2.5×10⁷ or more Tcons, approximately 3.0×10⁷ or more Tcons, approximately 3.5×10⁷ or more Tcons, approximately 4.0×10⁷ or more Tcons, approximately 4.5×10⁷ or more Tcons, approximately 5.0×10⁷ or more Tcons, approximately 5.5×10⁷ or more Tcons, approximately 6.0×10⁷ or more Tcons, approximately 6.5×10⁷ or more Tcons, approximately 7.0×10⁷ or more Tcons, approximately 7.5×10⁷ or more Tcons, approximately 8.0×10⁷ or more Tcons, approximately 8.5×10⁷ or more Tcons, approximately 9.0×10⁷ or more Tcons, approximately 9.5×10⁷ or more Tcons, approximately 1.0×10⁸ or more Tcons, approximately 1.5×10⁸ or more Tcons, approximately 2.0×10⁸ or more Tcons, approximately 2.5×10⁸ or more Tcons, approximately 3.0×10⁸ or more Tcons, approximately 3.5×10⁸ or more Tcons, approximately 4.0×10⁸ or more Tcons, approximately 4.5×10⁸ or more Tcons, approximately 5.0×10⁸ or more Tcons, approximately 5.5×10⁸ or more Tcons, approximately 6.0×10⁸ or more Tcons, approximately 6.5×10⁸ or more Tcons, approximately 7.0×10⁸ or more Tcons, approximately 7.5×10⁸ or more Tcons, approximately 8.0×10⁸ or more Tcons, approximately 8.5×10⁸ or more Tcons, approximately 9.0×10⁸ or more Tcons, approximately 9.5×10⁸ or more Tcons, approximately 1.0×10⁹ or more Tcons, approximately 1.5×10⁹ or more Tcons, approximately 2.0×10⁹ or more Tcons, approximately 2.5×10⁹ or more Tcons, approximately 3.0×10⁹ or more Tcons, approximately 3.5×10⁹ or more Tcons, approximately 4.0×10⁹ or more Tcons, approximately 4.5×10⁹ or more Tcons, approximately 5.0×10⁹ or more Tcons, approximately 5.5×10⁹ or more Tcons, or approximately 6.0×10⁹ or more Tcons.
Embodiment 10. The multi-component cellular therapy product of any one of embodiments 1-9, wherein the product further comprises a pharmaceutical composition comprising a dose of graft vs host disease (GVHD) prophylactic agent tacrolimus sufficient to maintain a trough blood level of approximately 5 ng/ml to approximately 10 ng/mL in the human subject receiving the product.
Embodiment 11. The multi-component cellular therapy product of embodiment 10, wherein the pharmaceutical composition comprises tacrolimus at a dose that ranges from approximately 0.01 mg per kilogram of body weight of the human subject receiving the product to approximately 0.50 mg per kilogram of body weight of the human subject receiving the product twice per day.
Embodiment 12. The multi-component cellular therapy product of embodiment 10 or embodiment 11, wherein the tacrolimus is dosed in an amount to maintain or that maintains a target blood level of approximately 1 ng/ml to approximately 10 ng/ml in the human subject receiving the product for approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the third population of CD45⁺ cells.
Embodiment 13. The multi-component cellular therapy product of any one of embodiments 1-12, wherein the first population of isolated CD45⁺ cells is formulated at a volume that ranges from approximately 5 mL to approximately 1 L.
Embodiment 14. The multi-component cellular therapy product of embodiment 13, wherein the first population of isolated CD45⁺ cells is formulated at a volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 125 mL, approximately 150 mL, approximately 175 mL, approximately 200 mL, approximately 225 mL, approximately 250 mL, approximately 275 mL, approximately 300 mL, approximately 325 mL, approximately 350 mL, approximately 375 mL, approximately 400 mL, approximately 425 mL, approximately 450 mL, approximately 475 mL, approximately 500 mL, approximately 525 mL, approximately 550 mL, approximately 575 mL, approximately 600 mL, approximately 625 mL, approximately 650 mL, approximately 675 mL, approximately 700 mL, approximately 725 mL, approximately 750 mL, approximately 775 mL, approximately 800 mL, approximately 825 mL, approximately 850 mL, approximately 875 mL, approximately 900 mL, approximately 925 mL, approximately 950 mL, approximately 975 mL, or approximately 1 L.
Embodiment 15. The multi-component cellular therapy product of any of one embodiments 1-14, wherein the second population of isolated CD45⁺ cells is formulated at a volume that ranges from approximately 5 mL to approximately 1L.
Embodiment 16. The multi-component cellular therapy product of embodiment 15, wherein the second population of isolated CD45⁺ cells is formulated at a volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 125 mL, approximately 150 mL, approximately 175 mL, approximately 200 mL, approximately 225 mL, approximately 250 mL, approximately 275 mL, approximately 300 mL, approximately 325 mL, approximately 350 mL, approximately 375 mL, approximately 400 mL, approximately 425 mL, approximately 450 mL, approximately 475 mL, approximately 500 mL, approximately 525 mL, approximately 550 mL, approximately 575 mL, approximately 600 mL, approximately 625 mL, approximately 650 mL, approximately 675 mL, approximately 700 mL, approximately 725 mL, approximately 750 mL, approximately 775 mL, approximately 800 mL, approximately 825 mL, approximately 850 mL, approximately 875 mL, approximately 900 mL, approximately 925 mL, approximately 950 mL, approximately 975 mL, or approximately 1 L.
Embodiment 17. The multi-component cellular therapy product of any one of embodiments 1-16, wherein the third population of isolated CD45⁺ cells is formulated at a volume that ranges from approximately 5 mL to approximately 1L.
Embodiment 18. The multi-component cellular therapy product of embodiment 17, wherein the third population of isolated CD45⁺ cells is formulated at a volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 125 mL, approximately 150 mL, approximately 175 mL, approximately 200 mL, approximately 225 mL, approximately 250 mL, approximately 275 mL, approximately 300 mL, approximately 325 mL, approximately 350 mL, approximately 375 mL, approximately 400 mL, approximately 425 mL, approximately 450 mL, approximately 475 mL, approximately 500 mL, approximately 525 mL, approximately 550 mL, approximately 575 mL, approximately 600 mL, approximately 625 mL, approximately 650 mL, approximately 675 mL, approximately 700 mL, approximately 725 mL, approximately 750 mL, approximately 775 mL, approximately 800 mL, approximately 825 mL, approximately 850 mL, approximately 875 mL, approximately 900 mL, approximately 925 mL, approximately 950 mL, approximately 975 mL, or approximately 1 L.
Embodiment 19. The multi-component cellular therapy product of any one of embodiments 1-18, wherein the neutral pH ranges from approximately 6.8 to approximately 7.6.
Embodiment 20. The multi-component cellular therapy product of any one of embodiments 1-19, wherein the excipient comprises a transport buffer.
Embodiment 21. The multi-component cellular therapy product of embodiment 20, wherein the transport buffer comprises approximately 120 to approximately 160 mEq sodium.
Embodiment 22. The multi-component cellular therapy product of embodiment 20 or embodiment 21, wherein the transport buffer comprises approximately 270 to approximately 320 mOsmol/L total.
Embodiment 23. The multi-component cellular therapy product of any one of embodiments 20-22, wherein the transport buffer is selected from the group consisting of: phosphate-buffered saline (PBS), human serum, PlasmaLyte, and any combination thereof.
Embodiment 24 The multi-component cellular therapy product of any one of embodiments 20-23, wherein the transport buffer further comprises approximately 0.1% weight by volume to approximately 10% weight by volume of a human carrier protein.
Embodiment 25. The multi-component cellular therapy product of embodiment 24, wherein the human carrier protein is selected from the group consisting of: human serum albumin (HSA), intravenous immune globulin (IVIG), AB serum, and any combination thereof.
Embodiment 26. The multi-component cellular therapy product of any one of embodiments 1-25, wherein any of the first single dose bag, the second single dose bad, and/or the third single dose bag is a polyvinyl chloride (PVC) transfer bag or an ethylene vinyl acetate (EVA) transfer bag.
Embodiment 27. The multi-component cellular therapy product of any one of embodiments 1-26, wherein the one or more cryoprotectants are selected from the group consisting of: sorbitol, dimethyl sulfoxide (DMSO), propylene glycol, glycerol, polyvinylpyrrolidone (PVP), and polyethylene glycol (PEG), serum, HSA, hetastarch, CRYOSTOR CS2, CRYOSTOR CS5, and CRYOSTOR CS10.
Embodiment 28. The multi-component cellular therapy product of any one of embodiments 1-27, wherein the first population of isolated CD45⁺ cells comprises from approximately 0.5% to approximately 5% granulocytes, from approximately 0.5% to approximately 3% monocytes, and from approximately 0.5% to approximately 2% lymphocytes.
Embodiment 29. The multi-component cellular therapy product of any one of embodiments 1-28, wherein the first population of isolated CD45⁺ cells comprises approximately 90% or more Tregs.
Embodiment 30. The multi-component cellular therapy product of any one of embodiments 1-29, wherein the third population of isolated CD45⁺ cells comprises from approximately 10% to approximately 35% monocytes, from approximately 20% to approximately 35% granulocytes, from approximately 0.5% to approximately 2% natural killer (NK) cells, from approximately 0.1% to approximately 20% CD34⁺ cells, and/or from approximately 0.1% to approximately 10% Tregs.
Embodiment 31. The multi-component cellular therapy product of any one of embodiments 1-30, wherein the first population of CD45⁺ cells comprises from less than approximately 5 EU to less than approximately 0.5 EU of endotoxins per mL of formulation.
Embodiment 32. The multi-component cellular therapy product of any one of embodiments 1-31, wherein the second population of CD45⁺ cells comprises from less than approximately 5 EU to less than approximately 0.5 EU of endotoxins per mL of formulation.
Embodiment 33. The multi-component cellular therapy product of any one of embodiments 1-32, wherein the third population of CD45⁺ cells comprises from less than approximately 5 EU to less than approximately 0.5 EU of endotoxins per mL of formulation.
Embodiment 34. The multi-component cellular therapy product of any one of embodiments 1-33, wherein the first population of isolated CD45⁺ cells, the second population of isolated CD45⁺ cells, and the third population of isolated CD45⁺ cells are from an allogeneic donor having at least one HLA mismatch relative to the human subject receiving the product.
Embodiment 35. The multi-component cellular therapy product of embodiment 34, wherein the HLA-mismatched donor is unrelated to the human subject receiving the product.
Embodiment 36. The multi-component cellular therapy product of embodiment 34, wherein the HLA-mismatched donor is related to the human subject receiving the product.
Embodiment 37. The multi-component cellular therapy product of any one of embodiments 34-36, wherein the at least one HLA mismatch is at an allele selected from the group consisting of: HLA-A, HLA-B, HLA-C, HLA-DRB1, and any combination thereof.
Embodiment 38. The multi-component cellular therapy product of any one of embodiments 34-37, wherein the cells having at least one HLA mismatch are from a donor that is 6/8 HLA-mismatched relative to the human subject receiving the product or is 7/8 HLA-mismatched relative to the human subject receiving the product.
Embodiment 39. The multi-component cellular therapy product of embodiment 38, wherein the cells having at least one HLA mismatch are from a donor that is 7/8 HLA-mismatched relative to the human subject receiving the product.
Embodiment 40. The multi-component cellular therapy product of embodiment 39, wherein the donor that is 7/8 HLA-mismatched relative to the human subject receiving the product has a mismatch in HLA-A.
Embodiment 41. The multi-component cellular therapy product of embodiment 39, wherein the donor that is 7/8 HLA-mismatched relative to the human subject receiving the product has a mismatch in HLA-B.
Embodiment 42. The multi-component cellular therapy product of embodiment 39, wherein the donor that is 7/8 HLA-mismatched relative to the human subject receiving the product has a mismatch in HLA-C.
Embodiment 43. The multi-component cellular therapy product of embodiment 39, wherein the donor that is 7/8 HLA-mismatched relative to the human subject receiving the product has a mismatch in HLA-DRB1.
Embodiment 44. The multi-component cellular therapy product of any one of embodiments 34-43, wherein the donor that has the at least one HLA mismatch relative to the human subject receiving the product has a mismatched HLA allele as a result of the donor being homozygous for the HLA allele while the human subject receiving the product is heterogeneous for the HLA allele.
Embodiment 45. The multi-component cellular therapy product of any one of embodiments 34-43, wherein the donor that has at least one HLA mismatch relative to the human subject receiving the product has a mismatched HLA allele as a result of the donor being heterozygous for the HLA allele while the human subject receiving the product is homozygous for the HLA allele.
Embodiment 46. The multi-component cellular therapy product of any one of embodiments 34-43, wherein the donor that has at least one HLA mismatch relative to the human subject receiving the product has a mismatched HLA allele as a result of both the donor and the human subject receiving the product being heterozygous for the HLA allele.
Embodiment 47. The multi-component cellular therapy product of any one of embodiments 1-46, wherein upon administration the first population of isolated CD45⁺ cells, the second population of isolated CD45⁺ cells, and the third population of isolated CD45⁺ cells decrease incidence of non-relapse mortality in the human subject receiving the product and/or increase overall survival in the human subject receiving the product compared to incidence of non-relapse mortality and/or over survival in a corresponding human subject that has been administered a standard myeloablative allogeneic hematopoietic stem cell transplant (alloHSCT) that is 7/8 HLA-mismatched relative to the corresponding human subject.
Embodiment 48. The multi-component cellular therapy product of any one of embodiments 1-47, wherein the Tregs are FOXP3+
Embodiment 49. The multi-component cellular therapy product of any one of embodiments 1-48, wherein the product further comprises approximately 1000 mg of mycophenolate mofetil (MMF).
Embodiment 50. The multi-component cellular therapy product of any one of embodiments 1-49, wherein the body weight of the human subject receiving the product is actual body weight.
Embodiment 51. The multi-component cellular therapy product of any one of embodiments 1-49, wherein the body weight of the human subject receiving the product is ideal body weight.
Embodiment 52. A method of treating a human subject having or suspected of having a hematologic malignancy, the method comprising administering to the human subject a multi-component pharmaceutical treatment comprising:
a) a solution comprising a first population of isolated CD45⁺ cells comprising hematopoietic stem and progenitor cells (HSPCs);
b) a solution comprising a second population of isolated CD45⁺ cells comprising regulatory T cells (Tregs);
c) a solution comprising a third population of isolated CD45⁺ cells wherein the third population of isolated CD45⁺ cells comprise CD3⁺ conventional T cells (Tcons); and
d) a solution comprising one or more doses of a graft vs host disease (GVHD) prophylactic agent, wherein the first population of isolated CD45⁺ cells, the second population of isolated CD45⁺ cells, and the third population of isolated CD45⁺ cells are obtained from a single allogeneic donor that has at least one HLA mismatch relative to the human subject, and wherein incidence of non-relapse mortality of the human subject is decreased after administration and/or overall survival of the human subject is increased after administration, relative to a corresponding human subject that has been administered a standard myeloablative allogeneic hematopoietic stem cell transplant (alloHSCT) from a donor that has at least one HLA mismatch relative to the corresponding human subject.
Embodiment 53. The method of embodiment 52, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN).
Embodiment 54. The method of embodiment 52 or embodiment 53, wherein the administering comprises infusing into the human subject the first population of isolated CD45⁺ cells, the second population of isolated CD45⁺ cells, and the third population of isolated CD45⁺ cells.
Embodiment 55. The method of any one of embodiments 52-54, wherein the third population of isolated CD45⁺ cells is administered at least approximately 12 hours after the first population of isolated CD45⁺ cells.
Embodiment 56. The method of any one of embodiments 52-55, wherein the third population of isolated CD45⁺ cells is administered from approximately 24 to approximately 120 hours after the first population of isolated CD45⁺ cells.
Embodiment 57. The method of any one of embodiments 52-56, wherein the third population of isolated CD45⁺ cells is administered from approximately 36 to approximately 72 hours after the first population of isolated CD45⁺ cells.
Embodiment 58. The method of any one of embodiments 52-57, wherein the third population of isolated CD45⁺ cells is administered at least approximately 12 hours after the second population of isolated CD45⁺ cells.
Embodiment 59. The method of any one of embodiments 52-58, wherein the third population of isolated CD45⁺ cells is administered from approximately 24 to approximately 120 hours after the second population of isolated CD45⁺ cells.
Embodiment 60. The method of any one of embodiments 52-59, wherein the third population of isolated CD45⁺ cells is administered from approximately 36 to approximately 72 hours after the second population of isolated CD45⁺ cells.
Embodiment 61. The method of any one of embodiments 52-60, wherein the HSPCs are CD34⁺.
Embodiment 62. The method of any one of embodiments 52-61, wherein the first population of isolated CD45⁺ cells comprises from approximately 5×10⁵ to approximately 2×10⁷ HSPCs per kilogram of actual or ideal body weight of the human subject.
Embodiment 63. The method of any one of embodiments 52-62, wherein the Tregs are CD4⁺ CD25⁺ CD127^{dim}.
Embodiment 64 The method of any one of embodiments 52-63, wherein in the second population of isolated CD45⁺ cells comprising Tregs more than approximately 90% of the CD45⁺ cells are Tregs.
Embodiment 65. The method of any one of embodiments 52-64, wherein the Tregs are CD4⁺CD25⁺CD127^{dim} or CD4⁺FOXP3⁺.
Embodiment 66. The method of any one of embodiments 52-65, wherein the second population of isolated CD45⁺ cells comprises from approximately 1×10⁵ to approximately 1×10⁷ Tregs per kilogram of actual or ideal body weight of the human subject.
Embodiment 67. The method of any one of embodiments 52-66, wherein the third population of isolated CD45⁺ cells comprises from approximately 1×10⁵ to approximately 1×10⁷ Tcons per kilogram of actual or ideal body weight of said human subject.
Embodiment 68. The method of any one of embodiments 52-67, wherein the human subject does not develop higher than stage 2 GVHD within approximately 100 days of the administering of the third population of isolated CD45⁺ cells.
Embodiment 69. The method of any one of embodiments 52-68, wherein the human subject does not develop higher than stage 2 GVHD within approximately 180 days or within approximately 200 days of the administering of the third population of isolated CD45⁺ cells.
Embodiment 70. The method of any one of embodiments 52-69, wherein the human subject does not develop higher than stage 2 GVHD within approximately 1 year of the administering of the third population of isolated CD45⁺ cells.
Embodiment 71. The method of any one of embodiments 52-70, wherein the human subject has previously been or is concurrently being treated for the hematologic malignancy.
Embodiment 72. The method of any one of embodiments 52-71, wherein the GVHD prophylactic agent is tacrolimus and is initially administered to the human subject at approximately 0.03 mg/kg human subject's actual or ideal body weight/day.
Embodiment 73. The method of any one of embodiments 52-72, wherein the GVHD prophylactic agent is initially administered from approximately 12 hours to approximately 24 hours after the administering of the third population of isolated CD45⁺ cells.
Embodiment 74. The method of embodiment 72 or embodiment 73, wherein the tacrolimus is administered in an amount to maintain a target blood level of at least approximately 3 ng/ml for at least approximately 20 days after administering the third population of isolated CD45⁺ cells.
Embodiment 75. The method of embodiment 74, wherein the tacrolimus is administered in an amount that maintains a target blood level of approximately 4 ng/ml or more for at least approximately 40 days after administering the third population of isolated CD45⁺ cells.
Embodiment 76. The method of any one of embodiments 72-75, wherein the tacrolimus is administered for at least approximately 60 days, at least approximately 90 days, at least approximately 120 days, or at most approximately 160 days after administering the third population of isolated CD45⁺ cells.
Embodiment 77. The method of any one of embodiments 52-76, wherein administration of the GVHD prophylactic agent is tapered starting at approximately 90 days after initial administration of the GVHD prophylactic agent.
Embodiment 78. The method of any one of embodiments 52-77, wherein the method further comprises administering approximately 1000 mg of mycophenolate mofetil (MMF).
Embodiment 79. The method of embodiment 78, wherein the MMF is initially administered from approximately 12 hours to approximately 24 hours after the administering of the third population of isolated CD45⁺ cells.
Embodiment 80. The method of embodiment 78 or embodiment 79, wherein administration of the MMF is tapered starting at approximately 30 days, at approximately 35 days, at approximately 40 days, at approximately 41 days, at approximately 42 days, at approximately 43 days, at approximately 44 days, at approximately 45 days, at approximately 46 days, at approximately 47 days, at approximately 48 days, at approximately 49 days, or at approximately 50 days after initial administration of the MMF.
Embodiment 81. The method of any one of embodiments 52-80, wherein the single allogeneic donor that has at least one HLA mismatch is unrelated to the human subject.
Embodiment 82. The method of any one of embodiments 52-81, wherein the single allogeneic donor that has at least one HLA mismatch is related to the human subject.
Embodiment 83. The method of any one of embodiments 52-82, wherein the method further comprises collecting one or more, or two or more mobilized peripheral blood donations from the donor.
Embodiment 84 The method of any one of embodiments 52-83, wherein the method further comprises collecting at most two mobilized peripheral blood donations from the donor.
Embodiment 85. The method of embodiment 83 or embodiment 84, wherein the peripheral blood donations are mobilized by granulocyte colony-stimulating factor (G-CSF), granulocyte macrophage colony-stimulating factor (GM-CSF), plerixafor, or any combination thereof.
Embodiment 86. The method of any one of embodiments 83-85, wherein at least one of the mobilized peripheral blood donations is processed and sorted using one or more immune-separation particles (ISPs) to enrich CD34⁺ cells and Tregs.
Embodiment 87. The method of embodiment 86, wherein the one or more ISPs comprise affinity reagents, optionally wherein the affinity reagents are immuno-magnetic separation particles, optionally wherein the immuno-magnetic separation particles are antibodies each conjugated to an iron-containing particle.
Embodiment 88. The method of embodiment 86 or embodiment 87, wherein an average number of ISP's per HSPC in the HSPC cell population is equal to or less than approximately 20,000.
Embodiment 89. The method of embodiment 86 or embodiment 87, wherein an average number of ISP's per HSPC in the HSPC cell population is from approximately 1000 to approximately 20,000.
Embodiment 90. The method of any one of embodiments 87-89, wherein the affinity reagents comprise a plurality of CD25-reagents that binds to one or more CD25 receptors on a Treg.
Embodiment 91. The method of embodiment 90, wherein an average number of ISPs per Treg in the second population of isolated CD45⁺ cells is equal or less than approximately 4000.
Embodiment 92. The method of embodiment 90, wherein an average number of ISPs per Treg in the second population of isolated CD45⁺ cells is from approximately 1500 to approximately 2500.
Embodiment 93. The method of any one of embodiments 52-92, wherein the at least one HLA mismatch is at an allele selected from the group consisting of: HLA-A, HLA-B, HLA-C, HLA-DRB1, and any combination thereof.
Embodiment 94. The method of any one of embodiments 52-93, wherein the allogeneic donor that has at least one HLA mismatch is 6/8 HLA-mismatched relative to the human subject or is 7/8 HLA-mismatched relative to the human subject.
Embodiment 95. The method of embodiment 94, wherein the allogeneic donor is 7/8 HLA-mismatched relative to the human subject.
Embodiment 96. The method of embodiment 95, wherein the allogeneic donor that is 7/8 HLA-mismatched relative to the human subject has a mismatch in HLA-A.
Embodiment 97. The method of embodiment 95, wherein the allogeneic donor that is 7/8 HLA-mismatched relative to the human subject has a mismatch in HLA-B.
Embodiment 98. The method of embodiment 95, wherein the allogeneic donor that is 7/8 HLA-mismatched relative to the human subject has a mismatch in HLA-C.
Embodiment 99. The method of embodiment 95, wherein the allogeneic donor that is 7/8 HLA-mismatched relative to the human subject has a mismatch in HLA-DRB1.
Embodiment 100. The method of any one of embodiments 52-99, wherein the allogeneic donor that has at least one HLA mismatch relative to the human subject has a mismatched HLA allele as a result of the allogeneic donor being homozygous for the HLA allele while the human subject is heterogeneous for the HLA allele.
Embodiment 101. The method of any one of embodiments 52-100, wherein the allogeneic donor that has at least one HLA mismatch relative to the human subject has a mismatched HLA allele as a result of the allogeneic donor being heterozygous for the HLA allele while the human subject is homozygous for the HLA allele.
Embodiment 102. The method of any one of embodiments 52-101, wherein the allogeneic donor that has at least one HLA mismatch relative to the human subject has a mismatched HLA allele as a result of both the allogeneic donor and the human subject being heterozygous for the HLA allele.
Embodiment 103. The method of any one of embodiments 52-102, wherein the method further comprises a conditioning regimen, wherein the conditioning regimen is administered before any of (a) to (d).
Embodiment 104. The method of embodiment 103, wherein the conditioning regimen is administered from approximately two days to approximately ten days before any of (a) to (d).
Embodiment 105. The method of embodiment 103 or embodiment 104, wherein the conditioning regimen is a myeloablative conditioning regimen.
Embodiment 106. The method of embodiment 105, wherein the myeloablative conditioning regimen comprises at least three conditioning reagents, wherein at least one conditioning reagent comprises thiotepa.
Embodiment 107. The method of embodiment 105, wherein the myeloablative conditioning regimen comprises one or more doses of busulfan, fludarabine and thiotepa.
Embodiment 108. The method of embodiment 107, wherein the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to
Embodiment 109. A multi-component pharmaceutical treatment to be administered to a human subject in need thereof, the multi-component treatment comprising:
a) a solution comprising a first population of isolated CD45⁺ cells comprising hematopoietic stem and progenitor cells (HSPCs) and granulocytes wherein at most approximately 10% of the first population of isolated CD45⁺ cells comprise granulocytes;
b) a solution comprising a second population of isolated CD45⁺ cells comprising regulatory T cells (Tregs);
c) a solution comprising a third population of isolated CD45⁺ cells wherein the third population of isolated CD45⁺ cells comprise at least approximately 20% CD3⁺ conventional T cells (Tcons), at least approximately 10% monocytes, and at least approximately 10% granulocytes; and
d) a solution comprising one or more doses of a graft vs host disease (GVHD) prophylactic agent, wherein the first population of isolated CD45⁺ cells, the second population of isolated CD45⁺ cells, and the third population of isolated CD45⁺ cells have at least one HLA mismatch relative to the human subject, and
wherein upon administration, the first population of isolated CD45⁺ cells, the second population of isolated CD45⁺ cells, and the third population of isolated CD45⁺ cells decrease incidence of non-relapse mortality in the human subject and/or increase overall survival in human subject compared to incidence of non-relapse mortality and/or over survival in a corresponding human subject that has been administered a standard myeloablative allogeneic hematopoietic stem cell transplant (alloHSCT) that has at least one HLA mismatch relative to the corresponding human subject.
Embodiment 110. The multi-component pharmaceutical treatment of embodiment 109, wherein the GVHD prophylactic agent is tacrolimus.
Embodiment 111. The multi-component pharmaceutical treatment of embodiment 109 or embodiment 110, wherein the one or more doses of the GVHD prophylactic agent comprises tacrolimus in an amount that maintains a target blood level of at least approximately 3 ng/ml for at least approximately 20 days after administration of the third population of isolated CD45⁺ cells.
Embodiment 112. The multi-component pharmaceutical treatment of any one of embodiments 109-111, wherein the treatment further comprises a conditioning regimen.
Embodiment 113. The multi-component pharmaceutical treatment of embodiment 112, wherein the conditioning regimen is a myeloablative conditioning regimen.
Embodiment 114. The multi-component pharmaceutical treatment of embodiment 113, wherein the myeloablative conditioning regimen comprises at least three conditioning reagents, wherein at least one conditioning reagent comprises thiotepa.
Embodiment 115. The multi-component pharmaceutical treatment of embodiment 113, wherein the myeloablative conditioning regimen comprises one or more doses of busulfan, fludarabine and thiotepa comprising from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.
Embodiment 116. The multi-component pharmaceutical treatment of any one of embodiments 109-115, wherein the HSPCs are CD34⁺.
Embodiment 117. The multi-component pharmaceutical treatment of any one of embodiments 109-116, wherein the Tregs are CD4⁺CD25⁺CD127^{dim}.
Embodiment 118. The multi-component pharmaceutical treatment of any one of embodiments 109-117, wherein in the second population of isolated CD45⁺ cells more than approximately 90% of the CD45⁺ cells are Tregs.
Embodiment 119. The multi-component pharmaceutical treatment of any one of embodiments 109-118, wherein the Tregs are CD4⁺CD25⁺CD127^{dim} or CD4⁺ FOXP3⁺.
Embodiment 120. The multi-component pharmaceutical treatment of any one of embodiments 109-119, wherein the first population of isolated CD45⁺ cells comprising HSPCs comprises from approximately 5×10⁵ to approximately 2×10⁷ HSPCs per kilogram of actual or ideal body weight of said human subject.
Embodiment 121. The multi-component pharmaceutical treatment of any one of embodiments 109-120, wherein the second population of CD45⁺ cells comprises from approximately 1×10⁵ to approximately 1×10⁷ Tregs per kilogram of actual or ideal body weight of said human subject.
Embodiment 122. The multi-component pharmaceutical treatment of any one of embodiments 109-121, wherein the third population of isolated CD45⁺ cells comprises from approximately 1×10⁵ to approximately 1×10⁷ Tcons per kilogram of actual or ideal body weight of said human subject.
Embodiment 123. The multi-component pharmaceutical treatment of any one of embodiments 109-122, wherein the first population of isolated CD45⁺ cells and the second population of CD45⁺ cells are formulated for administration prior to administration of the third population of isolated CD45⁺ cells.
Embodiment 124. The multi-component pharmaceutical treatment of any one of embodiments 109-123, wherein a first dose of the one or more doses of the GVHD prophylactic agent is formulated for administration subsequent to administration of the third population of isolated CD45⁺ cells.
Embodiment 125. The multi-component pharmaceutical treatment of any one of embodiments 109-124, wherein the treatment further comprises approximately 1000 mg of mycophenolate mofetil (MMF).
Embodiment 126. The multi-component pharmaceutical treatment of embodiment 125, wherein the MMF is formulated for administration from approximately 12 hours to approximately 24 hours subsequent to administering the third population of isolated CD45⁺ cells.
Embodiment 127. The multi-component pharmaceutical treatment of any one of embodiments 109-126, wherein the single allogeneic donor that has at least one HLA mismatch is unrelated to the human subject.
Embodiment 128. The multi-component pharmaceutical treatment of any one of embodiments 109-126, wherein the single allogeneic donor that has at least one HLA mismatch is related to the human subject.
Embodiment 129. The multi-component pharmaceutical treatment of any one of embodiments 109-128, wherein the at least one HLA mismatch is at an allele selected from the group consisting of: HLA-A, HLA-B, HLA-C, DRB-1, and any combination thereof.
Embodiment 130. The multi-component pharmaceutical treatment of any one of embodiments 109-129, wherein the allogeneic donor that has at least one HLA mismatch is 6/8 HLA-mismatched relative to the human subject or is 7/8 HLA-mismatched relative to the human subject.
Embodiment 131. The multi-component pharmaceutical treatment of embodiment 130, wherein the allogeneic donor is 7/8 HLA-mismatched relative to the human subject.
Embodiment 132. The multi-component pharmaceutical treatment of embodiment 131, wherein the allogeneic donor that is 7/8 HLA-mismatched relative to the human subject has a mismatch in HLA-A.
Embodiment 133. The multi-component pharmaceutical treatment of embodiment 131, wherein the allogeneic donor that is 7/8 HLA-mismatched relative to the human subject has a mismatch in HLA-B.
Embodiment 134. The multi-component pharmaceutical treatment of embodiment 131, wherein the allogeneic donor that is 7/8 HLA-mismatched relative to the human subject has a mismatch in HLA-C.
Embodiment 135. The multi-component pharmaceutical treatment of embodiment 131, wherein the allogeneic donor that is 7/8 HLA-mismatched relative to the human subject has a mismatch in HLA-DRB1.
Embodiment 136. The multi-component pharmaceutical treatment of any one of embodiments 109-135, wherein the allogeneic donor that has at least one HLA mismatch relative to the human subject has a mismatched HLA allele as a result of the allogeneic donor being homozygous for the HLA allele while the human subject is heterogeneous for the HLA allele.
Embodiment 137. The multi-component pharmaceutical treatment of any one of embodiments 109-135, wherein the allogeneic donor that has at least one HLA mismatch relative to the human subject has a mismatched HLA allele as a result of the allogeneic donor being heterozygous for the HLA allele while the human subject is homozygous for the HLA allele.
Embodiment 138. The multi-component pharmaceutical treatment of any one of embodiments 109-135, wherein the allogeneic donor that has at least one HLA mismatch relative to the human subject has a mismatched HLA allele as a result of both the allogeneic donor and the human subject being heterozygous for the HLA allele.
Embodiment 139. A method of treating a human subject having or suspected of having a hematologic malignancy, the method comprising administering to the human subject a multi-component pharmaceutical treatment comprising:
a) a myeloablative conditioning regimen comprising one or more doses of busulfan, fludarabine and thiotepa;
b) a solution comprising a first population of isolated CD45⁺ cells comprising hematopoietic stem and progenitor cells (HSPCs);
c) a solution comprising a second population of isolated CD45⁺ cells comprising regulatory T cells (Tregs);
d) a solution comprising a third population of isolated CD45⁺ cells wherein the third population of isolated CD45⁺ cells comprise CD3⁺ conventional T cells (Tcons); and
e) a solution comprising one or more doses of a graft vs host disease (GVHD) prophylactic agent, wherein the conditioning regimen is administered from approximately two days to approximately ten days before any of b) to e).
Embodiment 140. The method of embodiment 139, wherein the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.
Embodiment 141. The method of embodiment 139 or embodiment 140, wherein GVHD and relapse-free survival (GFRS) of the human subject increases by at least approximately 3.5-fold after administration, overall survival increases by at least approximately 1.25-fold after administration, and/or incidence of non-relapse mortality decreases by at least approximately 60% after administration, relative to a corresponding human subject that has been administered a standard myeloablative allogeneic hematopoietic stem cell transplant (alloHSCT).
Embodiment 142. A method of treating a human subject having or suspected of having a hematologic malignancy, the method comprising administering to the human subject a multi-component pharmaceutical treatment comprising:
a) a solution comprising a first population of isolated CD45⁺ cells comprising hematopoietic stem and progenitor cells (HSPCs);
b) a solution comprising a second population of isolated CD45⁺ cells comprising regulatory T cells (Tregs);
c) a solution comprising a third population of isolated CD45⁺ cells wherein the third population of isolated CD45⁺ cells comprise CD3⁺ conventional T cells (Tcons); and
d) a solution comprising one or more doses of a graft vs host disease (GVHD) prophylactic agent, wherein GVHD and relapse-free survival (GFRS) of the human subject increases by at least approximately 3.5-fold after administration, overall survival increased by at least approximately 1.25-fold after administration, and/or incidence of non-relapse mortality decreases by at least approximately 60% after administration, relative to a corresponding human subject that has been administered a standard myeloablative allogeneic hematopoietic stem cell transplant (alloHSCT).
Embodiment 143. The method of embodiment 142, wherein the multi-component pharmaceutical treatment further comprises a myeloablative conditioning regimen comprising one or more doses of busulfan, fludarabine and thiotepa.
Embodiment 144. The method of embodiment 143, wherein the conditioning regimen is administered from approximately two days to approximately ten days before any of (a) to (d).
Embodiment 145. The method of embodiment 143 or embodiment 144, wherein the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.
Embodiment 146. The method of any one of embodiments 139-145, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN).
Embodiment 147. The method of any one of embodiments 139-146, wherein the administering comprises infusing into the human subject the first population of isolated CD45⁺ cells, the second population of isolated CD45⁺ cells, and the third population of isolated CD45⁺ cells.
Embodiment 148. The method of any one of embodiments 139-147, wherein the third population of isolated CD45⁺ cells is administered at least approximately 12 hours after the first population of isolated CD45⁺ cells.
Embodiment 149. The method of any one of embodiments 139-148, wherein the third population of isolated CD45⁺ cells is administered from approximately 24 to approximately 120 hours after the first population of isolated CD45⁺ cells.
Embodiment 150. The method of any one of embodiments 139-149, wherein the third population of isolated CD45⁺ cells is administered from approximately 36 to approximately 72 hours after the first population of isolated CD45⁺ cells.
Embodiment 151. The method of any one of embodiments 139-150, wherein the third population of isolated CD45⁺ cells is administered at least approximately 12 hours after the second population of isolated CD45⁺ cells.
Embodiment 152. The method of any one of embodiments 139-151, wherein the third population of isolated CD45⁺ cells is administered from approximately 24 to approximately 120 hours after the second population of isolated CD45⁺ cells.
Embodiment 153. The method of any one of embodiments 139-152, wherein the third population of isolated CD45⁺ cells is administered from approximately 36 to approximately 72 hours after the second population of isolated CD45⁺ cells.
Embodiment 154. The method of any one of embodiments 139-153, wherein the HSPCs are CD34⁺.
Embodiment 155. The method of any one of embodiments 139-154, wherein the first population of isolated CD45⁺ cells comprises from approximately 5×10⁵ to approximately 2×107 HSPCs per kilogram of actual or ideal body weight of the human subject.
Embodiment 156. The method of any one of embodiments 139-155, wherein the Tregs are CD4⁺CD25⁺CD127^{dim}.
Embodiment 157. The method of any one of embodiments 139-156, wherein in the second population of isolated CD45⁺ cells more than approximately 90% of the CD45⁺ cells are Tregs.
Embodiment 158. The method of any one of embodiments 139-157, wherein the Tregs are CD4⁺CD25⁺CD127^{dim} or CD4⁺FOXP3⁺.
Embodiment 159. The method of any one of embodiments 139-158, wherein the second population of isolated CD45⁺ cells comprises from approximately 1×10⁵ to approximately 1×107 Tregs per kilogram of actual or ideal body weight of the human subject.
Embodiment 160. The method of any one of embodiments 139-159, wherein the third population of isolated CD45⁺ cells comprises from approximately 1×10⁵ to approximately 1×107 Tcons per kilogram of actual or ideal body weight of said human subject.
Embodiment 161. The method of any one of embodiments 139-160, wherein the human subject does not develop higher than stage 2 GVHD within approximately 100 days of the administering of the third population of isolated CD45⁺ cells.
Embodiment 162. The method of any one of embodiments 139-161, wherein the human subject does not develop higher than stage 2 GVHD within approximately 180 days or within approximately 200 days of the administering of the third population of isolated CD45⁺ cells.
Embodiment 163. The method of any one of embodiments 139-162, wherein the human subject does not develop higher than stage 2 GVHD within approximately 1 year of the administering of the third population of isolated CD45⁺ cells.
Embodiment 164. The method of any one of embodiments 139-163, wherein the human subject has previously been or is concurrently being treated for the hematologic malignancy.
Embodiment 165. The method of any one of embodiments 139-164, wherein the GVHD prophylactic agent is tacrolimus and is initially administered to the human subject at approximately 0.03 mg/kg human subject's actual or ideal body weight/day.
Embodiment 166. The method of any one of embodiments 139-165, wherein the GVHD prophylactic agent is initially administered from approximately 12 hours to approximately 24 hours after the administering of the third population of isolated CD45⁺ cells.
Embodiment 167. The method of embodiment 165 or embodiment 166, wherein the tacrolimus is administered in an amount to maintain a target blood level of at least approximately 3 ng/ml for at least approximately 20 days after administering the third population of isolated CD45⁺ cells.
Embodiment 168. The method of embodiment 167, wherein the tacrolimus is administered in an amount that maintains a target blood level of approximately 4 ng/ml or more for at least approximately 40 days after administering the third population of isolated CD45⁺ cells.
Embodiment 169. The method of any one of embodiments 165-168, wherein the tacrolimus is administered for at least approximately 60 days, at least approximately 90 days, at least approximately 120 days, or at most approximately 160 days after administering the third population of isolated CD45⁺ cells.
Embodiment 170. The method of any one of embodiments 139-169, wherein administration of the GVHD prophylactic agent is tapered starting at approximately 90 days after initial administration of the GVHD prophylactic agent.
Embodiment 171. The method of any one of embodiments 139-170, wherein the method further comprises collecting one or more, or two or more mobilized peripheral blood donations from the donor.
Embodiment 172. The method of any one of embodiments 139-171, wherein the method further comprises collecting at most two mobilized peripheral blood donations from the donor.
Embodiment 173. The method of embodiment 171 or embodiment 172, wherein the peripheral blood donations is mobilized by granulocyte colony-stimulating factor (G-CSF), granulocyte macrophage colony-stimulating factor (GM-CSF), plerixafor, or any combination thereof.
Embodiment 174. The method of any one of embodiments 171-173, wherein at least one of the mobilized peripheral blood donations is processed and sorted using one or more immune-separation particles (ISPs) to enrich CD34⁺ cells and Tregs.
Embodiment 175. The method of embodiment 174, wherein the one or more ISPs comprise affinity reagents, optionally wherein the affinity reagents are immuno-magnetic separation particles, optionally wherein the immuno-magnetic separation particles are antibodies each conjugated to an iron-containing particle.
Embodiment 176. The method of embodiment 174 or embodiment 175, wherein an average number of ISP's per HSPC in the HSPC cell population is equal to or less than approximately 20,000.
Embodiment 177. The method of embodiment 174 or embodiment 175, wherein an average number of ISP's per HSPC in the HSPC cell population is from approximately 1000 to approximately 20,000.
Embodiment 178. The method of any one of embodiments 174-177 wherein the affinity reagents comprise a plurality of CD25-reagents that binds to one or more CD25 receptors on a Treg.
Embodiment 179. The method of embodiment 178, wherein an average number of ISPs per T-reg cell in the Treg population is equal or less than approximately 4000.
Embodiment 180. The method of embodiment 178, wherein an average number of ISPs per T-reg cell in the Treg population is from approximately 1500 to approximately 2500.
Embodiment 181. A multi-component pharmaceutical treatment to be administered to a human subject in need thereof, the multi-component treatment comprising:
a) a myeloablative conditioning regimen comprising one or more doses of busulfan, fludarabine and thiotepa;
b) a solution comprising a first population of isolated CD45⁺ cells comprising hematopoietic stem and progenitor cells (HSPCs);
c) a solution comprising a second population of isolated CD45⁺ cells comprising regulatory T cells (Tregs);
d) a solution comprising a third population of isolated CD45⁺ cells wherein the third population of isolated CD45⁺ cells comprise CD3⁺ conventional T cells (Tcons); and
e) a solution comprising one or more doses of a graft vs host disease (GVHD) prophylactic agent, wherein the conditioning regimen is formulated for administration from approximately two days to approximately ten days before any of b) to e).
Embodiment 182. The multi-component pharmaceutical treatment of embodiment 181, wherein the one or more doses of busulfan, fludarabine and thiotepa comprise from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.
Embodiment 183. The multi-component pharmaceutical treatment of embodiment 181 or embodiment 182, wherein the GVHD prophylactic agent is tacrolimus.
Embodiment 184. The multi-component pharmaceutical treatment of any one of embodiments 181-183, wherein the one or more doses of the GVHD prophylactic agent comprise tacrolimus in an amount that maintains a target blood level of at least approximately 3 ng/ml for at least approximately 20 days after administration of the third population of isolated CD45⁺ cells.
Embodiment 185. The multi-component pharmaceutical treatment of any one of embodiments 181-184, wherein the HSPCs are CD34⁺.
Embodiment 186. The multi-component pharmaceutical treatment of any one of embodiments 181-185, wherein the Tregs are CD4⁺CD25⁺CD127^{dim}.
Embodiment 187. The multi-component pharmaceutical treatment of any one of embodiments 181-186, wherein the second population of CD45⁺ cells comprises CD45⁺ cells, wherein more than approximately 90% of the CD45⁺ cells are Tregs.
Embodiment 188. The multi-component pharmaceutical treatment of any one of embodiments 181-187, wherein the Tregs are CD4⁺CD25⁺CD127^{dim} or CD4⁺ FOXP3+.
Embodiment 189. The multi-component pharmaceutical treatment of any one of embodiments 181-188, wherein the first population of isolated CD45⁺ cells comprising HSPCs comprises from approximately 5×10⁵ to approximately 2×10⁷ HSPCs per kilogram of actual or ideal body weight of said human subject.
Embodiment 190. The multi-component pharmaceutical treatment of any one of embodiments 181-189, wherein the second population of CD45⁺ cells comprises from approximately 1×10⁵ to approximately 1×10⁷ Tregs per kilogram of actual or ideal body weight of said human subject.
Embodiment 191. The multi-component pharmaceutical treatment of any one of embodiments 181-190, wherein the third population of isolated CD45⁺ cells comprises from approximately 1×10⁵ to approximately 1×10⁷ Tcons per kilogram of actual or ideal body weight of said human subject.
Embodiment 192. The multi-component pharmaceutical treatment of any one of embodiments 181-191, wherein the first population of isolated CD45⁺ cells and the second population of CD45⁺ cells are formulated for administration prior to administration of the third population of isolated CD45⁺ cells.
Embodiment 193. The multi-component pharmaceutical treatment of any one of embodiments 181-192, wherein a first dose of the one or more doses of the GVHD prophylactic agent is formulated for administration subsequent to administration of the third population of isolated CD45⁺ cells.
Embodiment 194. A method of preparing a multi-component cellular therapy product comprising:
a) obtaining HSPC-mobilized peripheral blood apheresis from a donor that is allogeneic with respect to a human subject receiving the product;
b) combining a first apheresis sample with one or more cryoprotectants and cryopreserving the first apheresis sample, wherein the first apheresis sample comprises from approximately 1.0×10⁵ to approximately 4.0×10⁷ conventional CD3⁺ T cells (Tcons) per kilogram of body weight of the human subject receiving the product, from approximately 5.0×10⁵ to approximately 2.0×10⁸ Tcons, or from approximately 1.5×10⁷ to approximately 6.0×10⁹ Tcons;
c) sorting a second apheresis sample with one or more immune-separation particles (ISPs) specific for CD34 to obtain a CD34-enriched cell population and a CD34-depleted cell population, wherein the CD34-enriched cell population comprises from approximately 1.0×10⁵ to approximately 1.0×10⁸ CD34⁺ hematopoietic stem and progenitor cells (HSPCs) per kilogram of body weight of the human subject receiving the product, from approximately 5.0×10⁵ to approximately 5.0×10⁸ HSPCs, or from approximately 1.5×10⁷ to approximately 1.5×10¹⁰ HSPCs;
d) sorting the CD34-depleted cell population with one or more ISPs specific for CD25 to obtain a CD25-enriched cell population and a CD25-depleted cell population; and
e) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×10⁵ to approximately 2.0×107 CD4⁺CD25⁺CD127^{dim} regulatory T cells (Tregs) per kilogram of body weight of the human subject receiving the product, of from approximately 5.0×10⁵ to approximately 1.0×10⁸ isolated fresh Tregs, or from approximately 1.5×10⁷ to approximately 3.0×10⁹ isolated fresh Tregs, wherein the multi-component cellular therapy product comprises the Tcons, the HSPCs, and the Tregs.
Embodiment 195. A method of preparing a multi-component cellular therapy product comprising:
a) obtaining HSPC-mobilized peripheral blood apheresis from a donor that is allogeneic with respect to a human subject receiving the product;
b) sorting a first apheresis sample with one or more immune-separation particles (ISPs) specific for CD3 to obtain a CD3-enriched cell population and a CD3-depleted cell population, wherein the CD3-enriched cell population comprises from approximately 1.0×10⁵ to approximately 4.0×107 conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons;
c) sorting a second apheresis sample with one or more ISPs specific for CD34 to obtain a CD34-enriched cell population and a CD34-depleted cell population, wherein the CD34-enriched cell population comprises from approximately 1.0×10⁵ to approximately 1.0×10⁸ CD34⁺ hematopoietic stem and progenitor cells (HSPCs) per kilogram of body weight of the human subject receiving the product, from approximately 1.5×10⁷ to approximately 1.5×10¹⁰ HSPCs, or from approximately 5.0×10⁵ to approximately 5.0×10⁸ HSPCs;
d) sorting the CD34-depleted cell population with one or more ISPs specific for CD25 to obtain a CD25-enriched cell population and a CD25-depleted cell population; and
e) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×10⁵ to approximately 2.0×10⁷ CD4⁺CD25⁺CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, wherein the multi-component cellular therapy product comprises the Tcons, the HSPCs, and the Tregs.
Embodiment 196. The method of embodiment 195, wherein the method further comprises combining the CD3-enriched cell population with one or more cryoprotectants and cryopreserving the CD3-enriched cell population.
Embodiment 197. A method of preparing a multi-component cellular therapy product comprising:
a) obtaining HSPC-mobilized peripheral blood apheresis from a donor that is allogeneic with respect to a human subject receiving the product;
b) cryopreserving from a first apheresis sample a cell population comprising from approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons;
c) sorting a second apheresis sample with one or more immune-separation particles (ISPs) specific for CD25 to obtain a CD25-enriched cell population and a CD25-depleted cell population, and sorting the CD25-depleted population with one or more ISPs specific for CD34 to obtain a CD34-enriched cell population and a CD34-depleted cell population, wherein the CD34-enriched cell population comprises from approximately 1.0×10⁵ to approximately 1.0×10⁸ CD34⁺ hematopoietic stem and progenitor cells (HSPCs) per kilogram of body weight of the human subject receiving the product, from approximately 1.5×10⁷ to approximately 1.5×10¹⁰ HSPCs, or from approximately 5.0×10⁵ to approximately 5.0×10⁸ HSPCs; and
d) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×10⁵ to approximately 2.0×10⁷ CD4⁺CD25⁺CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, wherein the multi-component cellular therapy product comprises the Tcons, the HSPCs, and the Tregs.
Embodiment 198. The method of embodiment 197, wherein the method further comprises combining the cell population comprising Tons with one or more cryoprotectants prior to cryopreservation.
Embodiment 199. A method of preparing a multi-component cellular therapy product comprising:
a) obtaining HSPC-mobilized peripheral blood apheresis from a donor that is allogeneic with respect to a human subject receiving the product;
b) combining a first apheresis sample with one or more cryoprotectants and cryopreserving the first apheresis sample, wherein the first apheresis sample comprises from approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons;
c) sorting a second apheresis sample with one or more immune-separation particles (ISPs) specific for CD25 to obtain a CD25-enriched cell population and a CD25-depleted cell population, sorting the CD25-depleted population with one or more ISPs specific for CD34 to approximately obtain a CD34-enriched cell population and a CD34-depleted cell population, wherein the CD34-enriched cell population comprises from approximately 1.0×10⁵ to approximately 1.0×10⁸ CD34⁺ hematopoietic stem and progenitor cells (HSPCs) per kilogram of body weight of the human subject receiving the product, from approximately 1.5×10⁷ to approximately 1.5×10¹⁰ HSPCs, or from approximately 5.0×10⁵ to approximately 5.0×10⁸ HSPCs; and
d) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×10⁵ to approximately 2.0×10⁷ CD4⁺CD25⁺CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, wherein the multi-component cellular therapy product comprises the Tcons, the HSPCs, and the Tregs.
Embodiment 200. A method of preparing a multi-component cellular therapy product comprising:
a) obtaining HSPC-mobilized peripheral blood apheresis from a donor that is allogeneic with respect to a human subject receiving the product;
b) sorting a first apheresis sample with one or more immune-separation particles (ISPs) specific for CD3 to obtain a CD3-enriched cell population and a CD3-depleted cell population, wherein the CD3-enriched cell population comprises from approximately 1.0×10⁵ to approximately 4.0×10⁷ conventional CD3⁺ T cells (Tcons) per kilogram of body weight of the human subject receiving the product, from approximately 1.5×10⁷ to approximately 6.0×109 Tcons, from approximately 5.0×10⁵ to approximately 2.0×10⁸ Tcons;
c) sorting a second apheresis sample with one or more ISPs specific for CD25 to obtain a CD25-enriched cell population and a CD25-depleted cell population, sorting the CD25-depleted population with one or more ISPs specific for CD34 to obtain a CD34-enriched cell population and a CD34-depleted cell population, wherein the CD34-enriched cell population comprises from approximately 1.0×10⁵ to approximately 1.0×10⁸ CD34⁺ hematopoietic stem and progenitor cells (HSPCs) per kilogram of body weight of the human subject receiving the product, from approximately 1.5×10⁷ to approximately 1.5×10¹⁰ HSPCs, or from approximately 5.0×10⁵ to approximately 5.0×10⁸ HSPCs; and
d) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises from approximately 1.0×10⁵ to approximately 2.0×107 CD4⁺CD25⁺CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, wherein the multi-component cellular therapy product comprises the Tcons, the HSPCs, and the Tregs.
Embodiment 201. The method of embodiment 200, wherein the method further comprises combining the CD3-enriched cell population with one or more cryoprotectants and cryopreserving the CD3-enriched cell population.
Embodiment 202. A method of preparing a multi-component cellular therapy product comprising:
a) obtaining HSPC-mobilized peripheral blood apheresis from a donor that is allogeneic with respect to a human subject receiving the product;
b) cryopreserving a first apheresis sample, wherein the first apheresis sample comprises from approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons;
c) selecting from a second apheresis sample a CD34-enriched cell population comprising from approximately 1.0×10⁵ to approximately 1.0×10⁸ CD34⁺ hematopoietic stem and progenitor cells (HSPCs) per kilogram of body weight of the human subject receiving the product, from approximately 1.5×10⁷ to approximately 1.5×10¹⁰ HSPCs, from approximately 5.0×10⁵ to approximately 5.0×10⁸ HSPCs; and
d) selecting from the second apheresis sample a CD 25-enriched cell population that is CD4⁺ and CD127^{dim}
wherein the CD4⁺ and CD127^{dim} cell population comprises from approximately 1.0×10⁵ to approximately 2.0×10⁷ CD4⁺CD25⁺CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, wherein the multi-component cellular therapy product comprises the Tcons, the HSPCs, and the Tregs.
Embodiment 203. The method of embodiment 202, wherein the CD34-enriched cell population is selected by sorting the second apheresis sample with one or more ISPs specific for CD34 to obtain the CD34-enriched cell population and a CD34-depleted cell population.
Embodiment 204. The method of embodiment 203, wherein the CD25-enriched cell population is selected by soring the CD34-depleted cell population with one or more ISPs specific for CD25 to obtain the CD25-enriched cell population.
Embodiment 205. The method of embodiment 204, wherein the CD4⁺ and CD127^{dim} cell population is selected by sorting the CD25-enriched cell population with one or more ISPs specific for CD4 and one or more ISPs specific for CD127, and selecting the CD4⁺ and CD127^{dim} cell population.
Embodiment 206. The method of embodiment of any one of embodiments 202-205, wherein the method further comprises combining the first apheresis sample with one or more cryoprotectants prior to cryopreservation.
Embodiment 207. A method of preparing a multi-component cellular therapy product comprising:
a) obtaining HSPC-mobilized peripheral blood apheresis sample from a donor that is allogeneic with respect to a human subject receiving the product; and
b) selecting from the apheresis sample a first sample comprising from approximately 1.0×10⁵ to approximately 1.0×10⁸ CD34* hematopoietic stem and progenitor cells (HSPCs) per kilogram of body weight of the human subject receiving the product, from approximately 1.5×10⁷ to approximately 1.5×10¹⁰ HSPCs, from approximately 5.0×10⁵ to approximately 5.0×10⁸ HSPCs, a second sample comprising from approximately 1.0×10⁵ to approximately 2.0×10⁷ CD4⁺CD25⁺CD127^{dim} regulatory T cells (Tregs) per kilogram of body weight of the human subject receiving the product, from approximately 1.5×10⁷ to approximately 3.0×10⁹ Tregs, or from approximately 5.0×10⁵ to approximately 1.0×10⁸ Tregs, and a third sample comprising from approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons,
wherein the multi-component cellular therapy product comprises the Tcons, the HSPCs, and the Tregs.
Embodiment 208. The method of embodiment 207, wherein the HSPCs are selected by sorting the first sample with one or more ISPs specific for CD34 and selecting a CD34-enriched population.
Embodiment 209. The method of embodiment 207 or embodiment 208, wherein the Tregs are selected by soring the second sample with one or more ISPs specific for CD25 one or more ISPs specific for CD4 and one or more ISPs specific for CD127, and selecting the CD4⁺ and CD127^{dim} cell population.
Embodiment 210. The method of embodiment of any one of embodiments 207-209, wherein the third sample is cryopreserved.
Embodiment 211. The method of embodiment 210, wherein the third sample is combined with one or more cryoprotectants prior to cryopreservation.
Embodiment 212. The method of any one of embodiments 194-211, wherein the method further comprises formulating the Tcons, the Tregs, the HSPCs, or any combination thereof with one or more excipients at a neutral pH.
Embodiment 213. The method of any one of embodiments 194-211, wherein the method further comprises formulating the Tcons, the Tregs, and the HSPCs with one or more excipients at a neutral pH.
Embodiment 214. The method of any one of embodiments 194-211, wherein the method further comprises formulating the Tcons and the Tregs with one or more excipients at a neutral pH.
Embodiment 215. The method of any one of embodiments 194-211, wherein the method further comprises formulating the Tcons and the HSPCs with one or more excipients at a neutral pH.
Embodiment 216. The method of any one of embodiments 194-211, wherein the method further comprises formulating the Tregs and the HSPCs with one or more excipients at a neutral pH.
Embodiment 217. The method of any one of embodiments 194-216, wherein the method further comprises formulating the Tcons with one or more excipients at a neutral pH.
Embodiment 218. The method of any one of embodiments 194-217, wherein the method further comprises formulating the HSPCs with one or more excipients at a neutral pH.
Embodiment 219. The method of any one of embodiments 194-218, wherein the method further comprises formulating the Tregs with one or more excipients at a neutral pH.
Embodiment 220. The method of any one of embodiments 212-219, wherein the neutral pH ranges from approximately 6.8 to approximately 7.6.
Embodiment 221. The method of any one of embodiments 212-220, wherein the one or more excipients comprise one or more transport buffers.
Embodiment 222. The method of embodiment 221, wherein the one or more transport buffers comprise from approximately 120 to approximately 160 mEq sodium.
Embodiment 223. The method of embodiment 221 or embodiment 222, wherein the one or more transport buffers comprise from approximately 270 to approximately 320 mOsmol/L total.
Embodiment 224. The method of any one of embodiments 221-223, wherein the one or more transport buffers are independently selected from the group consisting of: phosphate-buffered saline (PBS), human serum, PlasmaLyte, and any combination thereof.
Embodiment 225. The method of any one of embodiments 221-224, wherein the one or more transport buffers further comprise from approximately 0.1% weight by volume to approximately 10% weight by volume of a human protein.
Embodiment 226. The method of embodiment 225, wherein the human protein is selected from the group consisting of: human serum albumin (HSA), intravenous immune globulin (IVIG), AB serum, and any combination thereof.
Embodiment 227. The method of any one of embodiments 194-226, wherein the one or more cryoprotectants are selected from the group consisting of: sorbitol, dimethyl sulfoxide (DMSO), propylene glycol, glycerol, polyvinylpyrrolidone (PVP), and polyethylene glycol (PEG), serum, HSA, hetastarch, CRYOSTOR CS2, CRYOSTOR CS5, and CRYOSTOR CS10.
Embodiment 228. The method of any one of embodiments 194-227, wherein the one or more ISPs comprise affinity reagents.
Embodiment 229. The method of embodiment 228, wherein the affinity reagents comprise immuno-magnetic separation particles, immuno-fluorescent separation particles, or any combination thereof.
Embodiment 230. The method of embodiment 229, wherein the immuno-magnetic separation particles are antibodies each conjugated to an iron-containing particle.
Embodiment 231. The method of embodiment 229 or embodiment 230, wherein the immuno-fluorescent separation particles are antibodies each conjugated to a fluorophore.
Embodiment 232. The method of any one of embodiments 228-231, wherein the one or more ISPs specific for CD34 are immuno-magnetic separation particles.
Embodiment 233. The method of embodiment 232, wherein an average number of ISP's per HSPC in the CD34-enriched cell population is from approximately 1,000 to approximately 20,000.
Embodiment 234. The method of any one of embodiments 228-232, wherein the one or more ISPs specific for CD25 are immuno-magnetic separation particles.
Embodiment 235. The method of embodiment 234, wherein an average number of ISP's per Treg in the CD25-enriched population is from approximately 1,500 to approximately 4,000.
Embodiment 236. The method of any one of embodiments 228-234, wherein the one or more ISPs specific for CD4 are immuno-fluorescent separation particles.
Embodiment 237. The method of any one of embodiments 228-236 wherein the one or more ISPs specific for CD127 immuno-fluorescent separation particles.
Embodiment 238. The method of any one of embodiments 194-237, wherein the HSPC-mobilized peripheral blood apheresis is mobilized by granulocyte colony-stimulating factor (G-CSF), granulocyte macrophage colony-stimulating factor (GM-CSF), plerixafor, or any combination thereof.
Embodiment 239. The method of any one of embodiments 194-238, wherein the Tcons sample comprises approximately 1.0×10⁵ or more Tcons per kilogram of body weight of the human subject receiving the product, approximately 2.0×10⁵ or more Tcons per kilogram of body weight of the human subject receiving the product, approximately 3.0×10⁵ or more Tcons per kilogram of body weight of the human subject receiving the product, approximately 4.0×10⁵ or more Tcons per kilogram of body weight of the human subject receiving the product, approximately 5.0×10⁵ or more Tcons per kilogram of body weight of the human subject receiving the product, approximately 6.0×10⁵ or more Tcons per kilogram of body weight of the human subject receiving the product, approximately 7.0×10⁵ or more Tcons per kilogram of body weight of the human subject receiving the product, approximately 8.0×10⁵ or more Tcons per kilogram of body weight of the human subject receiving the product, approximately 9.0×10⁵ or more Tcons per kilogram of body weight of the human subject receiving the product, approximately 1.0×10⁶ or more Tcons per kilogram of body weight of the human subject receiving the product, approximately 1.5×10⁶ or more Tcons per kilogram of body weight of the human subject receiving the product, approximately 2.0×10⁶ or more Tcons per kilogram of body weight of the human subject receiving the product, approximately 2.5×10⁶ or more Tcons per kilogram of body weight of the human subject receiving the product, approximately 3.0×10⁶ or more Tcons per kilogram of body weight of the human subject receiving the product, approximately 3.5×10⁶ or more Tcons per kilogram of body weight of the human subject receiving the product, approximately 4.0×10⁶ or more Tcons per kilogram of body weight of the human subject receiving the product, approximately 4.5×10⁶ or more Tcons per kilogram of body weight of the human subject receiving the product, approximately 5.0×10⁶ or more Tcons per kilogram of body weight of the human subject receiving the product, approximately 5.5×10⁶ or more Tcons per kilogram of body weight of the human subject receiving the product, approximately 6.0×10⁶ or more Tcons per kilogram of body weight of the human subject receiving the product, approximately 6.5×10⁶ or more Tcons per kilogram of body weight of the human subject receiving the product, approximately 7.0×10⁶ or more Tcons per kilogram of body weight of the human subject receiving the product, approximately 7.5×10⁶ or more Tcons per kilogram of body weight of the human subject receiving the product, approximately 8.0×10⁶ or more Tcons per kilogram of body weight of the human subject receiving the product, approximately 8.5×10⁶ or more Tcons per kilogram of body weight of the human subject receiving the product, approximately 9.0×10⁶ or Tcons per kilogram of body weight of the human subject receiving the product, approximately 9.5×10⁶ or more Tcons per kilogram of body weight of the human subject receiving the product, approximately 1.0×10⁷ or more Tcons per kilogram of body weight of the human subject receiving the product, approximately 1.5×10⁷ or more Tcons per kilogram of body weight of the human subject receiving the product, approximately 2.0×10⁷ or more Tcons per kilogram of body weight of the human subject receiving the product, approximately 2.5×10⁷ or more Tcons per kilogram of body weight of the human subject receiving the product, approximately 3.0×10⁷ or more Tcons per kilogram of body weight of the human subject receiving the product, approximately 3.5×10⁷ or more Tcons per kilogram of body weight of the human subject receiving the product, or approximately 4.0×10⁷ or more Tcons per kilogram of body weight of the human subject receiving the product.
Embodiment 240. The method of any one of embodiments 194-239, wherein the Tcons sample comprises approximately 5.0×10⁵ or more Tcons, approximately 6.0×10⁵ or more Tcons, approximately 7.0×10⁵ or more Tcons, approximately 8.0×10⁵ or more Tcons, approximately 9.0×10⁵ or more Tcons, approximately 1.0×10⁶ or more Tcons, approximately 1.5×10⁶ or more Tcons, approximately 2.0×10⁶ or more Tcons, approximately 2.5×10⁶ or more Tcons, approximately 3.0×10⁶ or more Tcons, approximately 3.5×10⁶ or more Tcons, approximately 4.0×10⁶ or more Tcons, approximately 4.5×10⁶ or more Tcons, approximately 5.0×10⁶ or more Tcons, approximately 5.5×10⁶ or more Tcons, approximately 6.0×10⁶ or more Tcons, approximately 6.5×10⁶ or more Tcons, approximately 7.0×10⁶ or more Tcons, approximately 7.5×10⁶ or more Tcons, approximately 8.0×10⁶ or more Tcons, approximately 8.5×10⁶ or more Tcons, approximately 9.0×10⁶ or Tcons, approximately 9.5×10⁶ or more Tcons, approximately 1.0×10⁷ or more Tcons, approximately 1.5×10⁷ or more Tcons, approximately 2.0×10⁷ or more Tcons, approximately 2.5×10⁷ or more Tcons, approximately 3.0×10⁷ or more Tcons, approximately 3.5×10⁷ or more Tcons, approximately 4.0×10⁷ or more Tcons, approximately 4.5×10⁷ or more Tcons, approximately 5.0×10⁷ or more Tcons, approximately 5.5×10⁷ or more Tcons, approximately 6.0×10⁷ or more Tcons, approximately 6.5×10⁷ or more Tcons, approximately 7.0×10⁷ or more Tcons, approximately 7.5×10⁷ or more Tcons, approximately 8.0×10⁷ or more Tcons, approximately 8.5×10⁷ or more Tcons, approximately 9.0×10⁷ or more Tcons, approximately 9.5×10⁷ or more Tcons, approximately 1.0×10⁸ or more Tcons, approximately 1.5×10⁸ or more Tcons, approximately 2.0×10⁸ or more Tcons, approximately 2.5×10⁸ or more Tcons, approximately 3.0×10⁸ or more Tcons, approximately 3.5×10⁸ or more Tcons, approximately 4.0×10⁸ or more Tcons, approximately 4.5×10⁸ or more Tcons, approximately 5.0×10⁸ or more Tcons, approximately 5.5×10⁸ or more Tcons, approximately 6.0×10⁸ or more Tcons, approximately 6.5×10⁸ or more Tcons, approximately 7.0×10⁸ or more Tcons, approximately 7.5×10⁸ or more Tcons, approximately 8.0×10⁸ or more Tcons, approximately 8.5×10⁸ or more Tcons, approximately 9.0×10⁸ or more Tcons, approximately 9.5×10⁸ or more Tcons, approximately 1.0×10⁹ or more Tcons, approximately 1.5×10⁹ or more Tcons, approximately 2.0×10⁹ or more Tcons, approximately 2.5×10⁹ or more Tcons, approximately 3.0×10⁹ or more Tcons, approximately 3.5×10⁹ or more Tcons, approximately 4.0×10⁹ or more Tcons, approximately 4.5×10⁹ or more Tcons, approximately 5.0×10⁹ or more Tcons, approximately 5.5×10⁹ or more Tcons, or approximately 6.0×10⁹ or more Tcons.
Embodiment 241. The method of any one of embodiments 194-240, wherein the HSPC sample comprises approximately 1.0×10⁵ or more HSPCs per kilogram of body weight of the human subject receiving the product, approximately 2.0×10⁵ or more HSPCs per kilogram of body weight of the human subject receiving the product, approximately 3.0×10⁵ or more HSPCs per kilogram of body weight of the human subject receiving the product, approximately 4.0×10⁵ or more HSPCs per kilogram of body weight of the human subject receiving the product, approximately 5.0×10⁵ or more HSPCs per kilogram of body weight of the human subject receiving the product, approximately 6.0×10⁵ or more HSPCs per kilogram of body weight of the human subject receiving the product, approximately 7.0×10⁵ or more HSPCs per kilogram of body weight of the human subject receiving the product, approximately 8.0×10⁵ or more HSPCs per kilogram of body weight of the human subject receiving the product, approximately 9.0×10⁵ or more HSPCs per kilogram of body weight of the human subject receiving the product, approximately 1.0×10⁶ or more HSPCs per kilogram of body weight of the human subject receiving the product, approximately 1.5×10⁶ or more HSPCs per kilogram of body weight of the human subject receiving the product, approximately 2.0×10⁶ or more HSPCs per kilogram of body weight of the human subject receiving the product, approximately 2.5×10⁶ or more HSPCs per kilogram of body weight of the human subject receiving the product, approximately 3.0×10⁶ or more HSPCs per kilogram of body weight of the human subject receiving the product, approximately 3.5×10⁶ or more HSPCs per kilogram of body weight of the human subject receiving the product, approximately 4.0×10⁶ or more HSPCs per kilogram of body weight of the human subject receiving the product, approximately 4.5×10⁶ or more HSPCs per kilogram of body weight of the human subject receiving the product, approximately 5.0×10⁶ or more HSPCs per kilogram of body weight of the human subject receiving the product, approximately 5.5×10⁶ or more HSPCs per kilogram of body weight of the human subject receiving the product, approximately 6.0×10⁶ or more HSPCs per kilogram of body weight of the human subject receiving the product, approximately 6.5×10⁶ or more HSPCs per kilogram of body weight of the human subject receiving the product, approximately 7.0×10⁶ or more HSPCs per kilogram of body weight of the human subject receiving the product, approximately 7.5×10⁶ or more HSPCs per kilogram of body weight of the human subject receiving the product, approximately 8.0×10⁶ or more HSPCs per kilogram of body weight of the human subject receiving the product, approximately 8.5×10⁶ or more HSPCs per kilogram of body weight of the human subject receiving the product, approximately 9.0×10⁶ or more HSPCs per kilogram of body weight of the human subject receiving the product, approximately 9.5×10⁶ or more HSPCs per kilogram of body weight of the human subject receiving the product, approximately 1.0×10⁷ or more HSPCs per kilogram of body weight of the human subject receiving the product, approximately 1.5×10⁷ or more HSPCs per kilogram of body weight of the human subject receiving the product, approximately 2.0×10⁷ or more HSPCs per kilogram of body weight of the human subject receiving the product, approximately 2.5×10⁷ or more HSPCs per kilogram of body weight of the human subject receiving the product, approximately 3.0×10⁷ or more HSPCs per kilogram of body weight of the human subject receiving the product, approximately 3.5×10⁷ or more HSPCs per kilogram of body weight of the human subject receiving the product, approximately 4.0×10⁷ or more HSPCs per kilogram of body weight of the human subject receiving the product, approximately 4.5×10⁷ or more HSPCs per kilogram of body weight of the human subject receiving the product, approximately 5.0×10⁷ or more HSPCs per kilogram of body weight of the human subject receiving the product, approximately 5.5×10⁷ or more HSPCs per kilogram of body weight of the human subject receiving the product, approximately 6.0×10⁷ or more HSPCs per kilogram of body weight of the human subject receiving the product, approximately 6.5×10⁷ or more HSPCs per kilogram of body weight of the human subject receiving the product, approximately 7.0×10⁷ or more HSPCs per kilogram of body weight of the human subject receiving the product, approximately 7.5×10⁷ or more HSPCs per kilogram of body weight of the human subject receiving the product, approximately 8.0×10⁷ or more HSPCs per kilogram of body weight of the human subject receiving the product, approximately 8.5×10⁷ or more HSPCs per kilogram of body weight of the human subject receiving the product, approximately 9.0×10⁷ or more HSPCs per kilogram of body weight of the human subject receiving the product, approximately 9.5×10⁷ or more HSPCs per kilogram of body weight of the human subject receiving the product, or approximately 1.0×10⁸ or more HSPCs per kilogram of body weight of the human subject receiving the product.
Embodiment 242. The method of any one of embodiments 194-241, wherein the HSPC sample comprises approximately 5.0×10⁵ or more HSPCs, approximately 6.0×10⁵ or more HSPCs, approximately 7.0×10⁵ or more HSPCs, approximately 8.0×10⁵ or more HSPCs, approximately 9.0×10⁵ or more HSPCs, approximately 1.0×10⁶ or more HSPCs, approximately 1.5×10⁶ or more HSPCs, approximately 2.0×10⁶ or more HSPCs, approximately 2.5×10⁶ or more HSPCs, approximately 3.0×10⁶ or more HSPCs, approximately 3.5×10⁶ or more HSPCs, approximately 4.0×10⁶ or more HSPCs, approximately 4.5×10⁶ or more HSPCs, approximately 5.0×10⁶ or more HSPCs, approximately 5.5×10⁶ or more HSPCs, approximately 6.0×10⁶ or more HSPCs, approximately 6.5×10⁶ or more HSPCs, approximately 7.0×10⁶ or more HSPCs, approximately 7.5×10⁶ or more HSPCs, approximately 8.0×10⁶ or more HSPCs, approximately 8.5×10⁶ or more HSPCs, approximately 9.0×10⁶ or more HSPCs, approximately 9.5×10⁶ or more HSPCs, approximately 1.0×10⁷ or more HSPCs, approximately 1.5×10⁷ or more HSPCs, approximately 2.0×10⁷ or more HSPCs, approximately 2.5×10⁷ or more HSPCs, approximately 3.0×10⁷ or more HSPCs, approximately 3.5×10⁷ or more HSPCs, approximately 4.0×10⁷ or more HSPCs, approximately 4.5×10⁷ or more HSPCs, approximately 5.0×10⁷ or more HSPCs, approximately 5.5×10⁷ or more HSPCs, approximately 6.0×10⁷ or more HSPCs, approximately 6.5×10⁷ or more HSPCs, approximately 7.0×10⁷ or more HSPCs, approximately 7.5×10⁷ or more HSPCs, approximately 8.0×10⁷ or more HSPCs, approximately 8.5×10⁷ or more HSPCs, approximately 9.0×10⁷ or more HSPCs, approximately 9.5×10⁷ or more HSPCs, approximately 1.0×10⁸ or more HSPCs, approximately 1.5×10⁸ or more HSPCs, approximately 2.0×10⁸ or more HSPCs, approximately 2.5×10⁸ or more HSPCs, approximately 3.0×10⁸ or more HSPCs, approximately 3.5×10⁸ or more HSPCs, approximately 4.0×10⁸ or more HSPCs, approximately 4.5×10⁸ or more HSPCs, approximately 5.0×10⁸ or more HSPCs, approximately 5.5×10⁸ or more HSPCs, approximately 6.0×10⁸ or more HSPCs, approximately 6.5×10⁸ or more HSPCs, approximately 7.0×10⁸ or more HSPCs, approximately 7.5×10⁸ or more HSPCs, approximately 8.0×10⁸ or more HSPCs, approximately 8.5×10⁸ or more HSPCs, approximately 9.0×10⁸ or more HSPCs, approximately 9.5×10⁸ or more HSPCs, approximately 1.0×10⁹ or more HSPCs, approximately 1.5×10⁹ or more HSPCs, approximately 2.0×10⁹ or more HSPCs, approximately 2.5×10⁹ or more HSPCs, approximately 3.0×10⁹ or more HSPCs, approximately 3.5×10⁹ or more HSPCs, approximately 4.0×10⁹ or more HSPCs, approximately 4.5×10⁹ or more HSPCs, approximately 5.0×10⁹ or more HSPCs, approximately 5.5×10⁹ or more HSPCs, approximately 6.0×10⁹ or more HSPCs, approximately 6.5×10⁹ or more HSPCs, approximately 7.0×10⁹ or more HSPCs, approximately 7.5×10⁹ or more HSPCs, approximately 8.0×10⁹ or more HSPCs, approximately 8.5×10⁹ or more HSPCs, approximately 9.0×10⁹ or more HSPCs, approximately 9.5×10⁹ or more HSPCs, approximately 1.0×10¹⁰ or more HSPCs, or approximately 1.5×10¹⁰ or more HSPCs.
Embodiment 243. The method of any one of embodiments 194-242, wherein the Tregs sample comprises approximately 1.0×10⁵ or more Tregs per kilogram of body weight of the human subject receiving the product, approximately 2.0×10⁵ or more Tregs per kilogram of body weight of the human subject receiving the product, approximately 3.0×10⁵ or more Tregs per kilogram of body weight of the human subject receiving the product, approximately 4.0×10⁵ or more Tregs per kilogram of body weight of the human subject receiving the product, approximately 5.0×10⁵ or more Tregs per kilogram of body weight of the human subject receiving the product, approximately 6.0×10⁵ or more Tregs per kilogram of body weight of the human subject receiving the product, approximately 7.0×10⁵ or more Tregs per kilogram of body weight of the human subject receiving the product, approximately 8.0×10⁵ or more Tregs per kilogram of body weight of the human subject receiving the product, approximately 9.0×10⁵ or more Tregs per kilogram of body weight of the human subject receiving the product, approximately 1.0×10⁶ or more Tregs per kilogram of body weight of the human subject receiving the product, approximately 1.5×10⁶ or more Tregs per kilogram of body weight of the human subject receiving the product, approximately 2.0×10⁶ or more Tregs per kilogram of body weight of the human subject receiving the product, approximately 2.5×10⁶ or more Tregs per kilogram of body weight of the human subject receiving the product, approximately 3.0×10⁶ or more Tregs per kilogram of body weight of the human subject receiving the product, approximately 3.5×10⁶ or more Tregs per kilogram of body weight of the human subject receiving the product, approximately 4.0×10⁶ or more Tregs per kilogram of body weight of the human subject receiving the product, approximately 4.5×10⁶ or more Tregs per kilogram of body weight of the human subject receiving the product, approximately 5.0×10⁶ or more Tregs per kilogram of body weight of the human subject receiving the product, approximately 5.5×10⁶ or more Tregs per kilogram of body weight of the human subject receiving the product, approximately 6.0×10⁶ or more Tregs per kilogram of body weight of the human subject receiving the product, approximately 6.5×10⁶ or more Tregs per kilogram of body weight of the human subject receiving the product, approximately 7.0×10⁶ or more Tregs per kilogram of body weight of the human subject receiving the product, approximately 7.5×10⁶ or more Tregs per kilogram of body weight of the human subject receiving the product, approximately 8.0×10⁶ or more Tregs per kilogram of body weight of the human subject receiving the product, approximately 8.5×10⁶ or more Tregs per kilogram of body weight of the human subject receiving the product, approximately 9.0×10⁶ or more Tregs per kilogram of body weight of the human subject receiving the product, approximately 9.5×10⁶ or more Tregs per kilogram of body weight of the human subject receiving the product, approximately 1.0×10⁷ or more Tregs per kilogram of body weight of the human subject receiving the product, approximately 1.5×10⁷ or more Tregs per kilogram of body weight of the human subject receiving the product, or approximately 2.0×10⁷ or more Tregs per kilogram of body weight of the human subject receiving the product.
Embodiment 244. The method of any one of embodiments 194-243, wherein the Tregs sample comprises approximately 5.0×10⁵ or more Tregs, approximately 6.0×10⁵ or more Tregs, approximately 7.0×10⁵ or more Tregs, approximately 8.0×10⁵ or more Tregs, approximately 9.0×10⁵ or more Tregs, approximately 1.0×10⁶ or more Tregs, approximately 1.5×10⁶ or more Tregs, approximately 2.0×10⁶ or more Tregs, approximately 2.5×10⁶ or more Tregs, approximately 3.0×10⁶ or more Tregs, approximately 3.5×10⁶ or more Tregs, approximately 4.0×10⁶ or more Tregs, approximately 4.5×10⁶ or more Tregs, approximately 5.0×10⁶ or more Tregs, approximately 5.5×10⁶ or more Tregs, approximately 6.0×10⁶ or more Tregs, approximately 6.5×10⁶ or more Tregs, approximately 7.0×10⁶ or more Tregs, approximately 7.5×10⁶ or more Tregs, approximately 8.0×10⁶ or more Tregs, approximately 8.5×10⁶ or more Tregs, approximately 9.0×10⁶ or more Tregs, approximately 9.5×10⁶ or more Tregs, approximately 1.0×10⁷ or more Tregs, approximately 1.5×10⁷ or more Tregs, approximately 2.0×10⁷ or more Tregs, approximately 2.5×10⁷ or more Tregs, approximately 3.0×10⁷ or more Tregs, approximately 3.5×10⁷ or more Tregs, approximately 4.0×10⁷ or more Tregs, approximately 4.5×10⁷ or more Tregs, approximately 5.0×10⁷ or more Tregs, approximately 5.5×10⁷ or more Tregs, approximately 6.0×10⁷ or more Tregs, approximately 6.5×10⁷ or more Tregs, approximately 7.0×10⁷ or more Tregs, approximately 7.5×10⁷ or more Tregs, approximately 8.0×10⁷ or more Tregs, approximately 8.5×10⁷ or more Tregs, approximately 9.0×10⁷ or more Tregs, approximately 9.5×10⁷ or more Tregs, approximately 1.0×10⁸ or more Tregs, approximately 1.5×10⁸ or more Tregs, approximately 2.0×10⁸ or more Tregs, approximately 2.5×10⁸ or more Tregs, approximately 3.0×10⁸ or more Tregs, approximately 3.5×10⁸ or more Tregs, approximately 4.0×10⁸ or more Tregs, approximately 4.5×10⁸ or more Tregs, approximately 5.0×10⁸ or more Tregs, approximately 5.5×10⁸ or more Tregs, approximately 6.0×10⁸ or more Tregs, approximately 6.5×10⁸ or more Tregs, approximately 7.0×10⁸ or more Tregs, approximately 7.5×10⁸ or more Tregs, approximately 8.0×10⁸ or more Tregs, approximately 8.5×10⁸ or more Tregs, approximately 9.0×10⁸ or more Tregs, approximately 9.5×10⁸ or more Tregs, approximately 1.0×10⁹ or more Tregs, approximately 1.5×10⁹ or more Tregs, approximately 2.0×10⁹ or more Tregs, approximately 2.5×10⁹ or more Tregs, or approximately 3.0×10⁹ or more Tregs.
Embodiment 245. The method of any one of embodiments 194-244, wherein the Tregs are FOXP3⁺.
Embodiment 246. The method of any one of embodiments 194-245, wherein the allogeneic donor is 7/8 HLA-mismatched relative to the human subject receiving the product.
Embodiment 247. The method of embodiment 246, wherein the 7/8 HLA-mismatched donor is unrelated to the human subject receiving the product.
Embodiment 248. The method of embodiment 246, wherein the 7/8 HLA-mismatched donor is related to the human subject receiving the product.
Embodiment 249. The method of any one of embodiments 246-248, wherein the allogeneic donor that is 7/8 HLA-mismatched relative to the human subject receiving the product has a mismatch in HLA-A, HLA-B, HLA-C, or HLA-DRB1.
Embodiment 250. The method of any one of embodiments 194-249, wherein the HSPC sample comprises from less than approximately 5 EU to less than approximately 0.5 EU of endotoxins per mL of multi-component cellular therapy product.
Embodiment 251. The method of any one of embodiments 194-250, wherein the Treg sample comprises from less than approximately 5 EU to less than approximately 0.5 EU of endotoxins per mL of multi-component cellular therapy product.
Embodiment 252. The method of any one of embodiments 194-251, wherein the Tcon sample comprises from less than approximately 5 EU to less than approximately 0.5 EU of endotoxins per mL of multi-component cellular therapy product.
Embodiment 253. A multi-component cellular therapy product produced by the method of any of embodiments 194-252.
Embodiment 254. The method of any one of embodiments 194-252, wherein the multi-component cellular therapy product is the multi-component cellular therapy product of any one of embodiments 1-51.
Embodiment 255. The method of any one of embodiments 194-252, wherein the multi-component cellular therapy product is the multi-component pharmaceutical treatment of any one of embodiments 109-138 or 181-193.
Embodiment 256. A method of treating a human subject having or suspected of having acute leukemia in complete remission, the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 257. A method of treating a human subject having or suspected of having acute leukemia in complete remission, the method comprising administering to the human subject the multi-component cellular therapy product of any one of embodiments 1-51 or the multi-component pharmaceutical treatment of any one of embodiments 109-138 or 181-193.
Embodiment 258. The method of embodiment 255 or embodiment 257, wherein the acute leukemia is in complete remission with incomplete hematologic recovery.
Embodiment 259. The method of any one of embodiments 256-258, wherein minimal residual disease is present in the human subject.
Embodiment 260. The method of any one of embodiments 256-258, wherein minimal residual disease is absent in the human subject.
Embodiment 261. The method of any one of embodiments 256-260, wherein the acute leukemia is categorized as intermediate-risk to high-risk acute leukemia.
Embodiment 262. The method of any one of embodiments 256-260, wherein the acute leukemia is categorized as very high-risk acute leukemia.
Embodiment 263. A method of treating a human subject having or suspected of having active leukemia, the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 264. A method of treating a human subject having or suspected of having active leukemia, the method comprising administering to the human subject the multi-component cellular therapy product of any one of embodiments 1-51 or the multi-component pharmaceutical treatment of any one of embodiments 109-138 or 181-193.
Embodiment 265. A method of treating a human subject having or suspected of having primary refractory acute leukemia or acute leukemia with minimal residual disease, the method comprising administering to the human subject a multi-component cellular therapy comprising:
(a) a solution comprising a first population of isolated CD45⁺ cells comprising CD34⁺ hematopoietic stem and progenitor cells (HSPCs);
(b) a solution comprising a second population of isolated CD45⁺ cells comprising fresh CD4⁺CD25⁺CD127^{dim} regulatory T cells (Tregs); and
(c) a solution comprising a third population of isolated CD45⁺ cells comprising conventional CD3⁺ T cells (Tcons).
Embodiment 266. A method of treating a human subject having or suspected of having primary refractory acute leukemia or acute leukemia with minimal residual disease, the method comprising administering to the human subject the multi-component cellular therapy product of any one of embodiments 1-51 or the multi-component pharmaceutical treatment of any one of embodiments 109-138 or 181-193.
Embodiment 267. The method of any one of embodiments 256-266, wherein the acute leukemia is acute myeloid leukemia (AML), acute lymphoid leukemia (ALL), and/or mixed phenotype acute leukemia (MPAL).
Embodiment 268. A method of treating a human subject having or suspected of having low-risk acute myeloid leukemia (AML), the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 269. A method of treating a human subject having or suspected of having low-risk acute myeloid leukemia (AML), the method comprising administering to the human subject the multi-component cellular therapy product of any one of embodiments 1-51 or the multi-component pharmaceutical treatment of any one of embodiments 109-138 or 181-193.
Embodiment 270. A method of treating a human subject having or suspected of having high-risk acute myeloid leukemia (AML) in complete remission, the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 271. A method of treating a human subject having or suspected of having high-risk acute myeloid leukemia (AML) in complete remission, the method comprising administering to the human subject the multi-component cellular therapy product of any one of embodiments 1-51 or the multi-component pharmaceutical treatment of any one of embodiments 109-138 or 181-193.
Embodiment 272. The method of embodiment 270 or embodiment 271, wherein the high risk AML comprises a complex karyotype with 3 or more clonal chromosomal abnormalities.
Embodiment 273. The method of embodiment 272, wherein the 3 or more clonal chromosomal abnormalities are each selected from the group consisting of: monosomal karyotype-5, 5q-, -7 or 7q-, t(11q23, t(9;11), inv(3), t(3,3)t(6;9)t(9;22), normal karyotype with a fms-like tyrosine kinase 3 (FLT3)-ITD mutation, and any combination thereof.
Embodiment 274. A method of treating a human subject having or suspected of having chronic myelogenous leukemia (CML), the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 275. A method of treating a human subject having or suspected of having chronic myelogenous leukemia (CML), the method comprising administering to the human subject the multi-component cellular therapy product of any one of embodiments 1-51 or the multi-component pharmaceutical treatment of any one of embodiments 109-138 or 181-193.
Embodiment 276. The method of embodiment 274 or embodiment 275, wherein the CML is in blast phase.
Embodiment 277. The method of any one of embodiments 274-276, wherein the CML is in second chronic phase.
Embodiment 278. The method of embodiment 274 or embodiment 275, wherein the CML is in chronic phase.
Embodiment 279. The method of any one of embodiments 274-278, wherein the CML is accelerated.
Embodiment 280. The method of any one of embodiments 274-279, wherein the CML has a history of blast crisis.
Embodiment 281. The method of any one of embodiments 274-280, wherein the CML is resistant to or intolerant of one or more first-generation or second-generation tyrosine kinase inhibitors (TKIs).
Embodiment 282. A method of treating a human subject having or suspected of having high-risk myelodysplastic syndrome, the method comprising administering to the human subject a multi-component cellular therapy comprising:
(a) a solution comprising a first population of isolated CD45⁺ cells comprising CD34⁺ hematopoietic stem and progenitor cells (HSPCs);
(b) a solution comprising a second population of isolated CD45⁺ cells comprising fresh CD4⁺CD25⁺CD127^{dim} regulatory T cells (Tregs); and
(c) a solution comprising a third population of isolated CD45⁺ cells comprising conventional CD3⁺ T cells (Tcons).
Embodiment 283 A method of treating a human subject having or suspected of having high-risk myelodysplastic syndrome, the method comprising administering to the human subject the multi-component cellular therapy product of any one of embodiments 1-51 or the multi-component pharmaceutical treatment of any one of embodiments 109-138 or 181-193.
Embodiment 284. A method of treating a human subject having or suspected of having therapy-related myelodysplastic syndrome and/or secondary myelodysplastic syndrome, the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 285. A method of treating a human subject having or suspected of having therapy-related myelodysplastic syndrome and/or secondary myelodysplastic syndrome, the method comprising administering to the human subject the multi-component cellular therapy product of any one of embodiments 1-51 or the multi-component pharmaceutical treatment of any one of embodiments 109-138 or 181-193.
Embodiment 286. The method of any one of embodiments 282-285, wherein the human subject has active disease at time of treatment with the multi-component cellular therapy.
Embodiment 287. The method of any one of embodiments 282-286, wherein the human subject has approximately 10% or less blast burden in their bone marrow.
Embodiment 288. The method of embodiment 284 or embodiment 285, wherein the therapy-related myelodysplastic syndrome and/or secondary myelodysplastic syndrome is in complete remission and is categorized as intermediate-risk to high-risk.
Embodiment 289. A method of treating a human subject having or suspected of having a myeloproliferative syndrome, the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 290. A method of treating a human subject having or suspected of having a myeloproliferative syndrome, the method comprising administering to the human subject the multi-component cellular therapy product of any one of embodiments 1-51 or the multi-component pharmaceutical treatment of any one of embodiments 109-138 or 181-193.
Embodiment 291. A method of treating a human subject having or suspected of having non-Hodgkin lymphoma with poor risk features not suitable for autologous hematopoietic cell transplant (HCT), the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 292. A method of treating a human subject having or suspected of having non-Hodgkin lymphoma with poor risk features not suitable for autologous hematopoietic cell transplant (HCT), the method comprising administering to the human subject the multi-component cellular therapy product of any one of embodiments 1-51 or the multi-component pharmaceutical treatment of any one of embodiments 109-138 or 181-193.
Embodiment 293. A method of treating a human subject having or suspected of having multiple sclerosis, the method comprising administering to the human subject a multi-component cellular therapy comprising:
a) a solution comprising a first population of isolated CD45⁺ cells comprising CD34⁺ hematopoietic stem and progenitor cells (HSPCs);
b) a solution comprising a second population of isolated CD45⁺ cells comprising fresh CD4⁺CD25⁺CD127^{dim} regulatory T cells (Tregs); and
c) a solution comprising a third population of isolated CD45⁺ cells comprising conventional CD3⁺ T cells (Tcons).
Embodiment 294. A method of treating a human subject having or suspected of having multiple sclerosis, the method comprising administering to the human subject the multi-component cellular therapy product of any one of embodiments 1-51 or the multi-component pharmaceutical treatment of any one of embodiments 109-138 or 181-193.
Embodiment 295. The method of embodiment 293 or embodiment 294, wherein the multiple sclerosis is primary progressive multiple sclerosis.
Embodiment 296. The method of embodiment 293 or embodiment 294, wherein the multiple sclerosis is chronic progressive multiple sclerosis.
Embodiment 297. The method of embodiment 293 or embodiment 294, wherein the multiple sclerosis is secondary progressive multiple sclerosis.
Embodiment 298. The method of embodiment 293 or embodiment 294, wherein the multiple sclerosis is relapsing-remitting multiple sclerosis.
Embodiment 299. The method of embodiment 293 or embodiment 294, wherein the multiple sclerosis is acute relapsing multiple sclerosis.
Embodiment 300. The method of any one of embodiments 256-299, wherein the third population of isolated CD45⁺ cells is administered approximately 12 hours or more after the first population of isolated CD45⁺ cells.
Embodiment 301. The method of any one of embodiments 256-300, wherein the third population of isolated CD45⁺ cells is administered from approximately 24 to approximately 120 hours after the first population of isolated CD45⁺ cells.
Embodiment 302. The method of any one of embodiments 256-301, wherein the third population of isolated CD45⁺ cells is administered from approximately 36 to approximately 72 hours after the first population of isolated CD45⁺ cells.
Embodiment 303. The method of any one of embodiments 256-302, wherein third population of isolated CD45⁺ cells is administered approximately 12 hours, approximately 15 hours, approximately 18 hours, approximately 21 hours, approximately, 24 hours, approximately 27 hours, approximately 30 hours, approximately 33 hours, approximately 36 hours, approximately 39 hours, approximately 42 hours, approximately 45 hours, approximately 48 hours, approximately 51 hours, approximately 54 hours, approximately 57 hours, approximately 60 hours, approximately 63 hours, approximately 66 hours, approximately 69 hours, approximately 72 hours, approximately 75 hours, approximately 78 hours, approximately 81 hours, approximately 84 hours, approximately 87 hours, approximately 90 hours, approximately 93 hours, approximately 96 hours, approximately 99 hours, approximately 102 hours, approximately 105 hours, approximately 108 hours, approximately 111 hours, approximately 114 hours, approximately 117 hours, approximately 120 hours after administration of the first population of isolated CD45⁺ cells.
Embodiment 304. The method of any one of embodiments 256-303, wherein the third population of isolated CD45⁺ cells is administered approximately 12 hours or more after the second population of isolated CD45⁺ cells.
Embodiment 305. The method of any one of embodiments 256-304, wherein the third population of isolated CD45⁺ cells is administered from approximately 24 to approximately 120 hours after the second population of isolated CD45⁺ cells.
Embodiment 306. The method of any one of embodiments 256-305, wherein the third population of isolated CD45⁺ cells is administered from approximately 36 to approximately 72 hours after the second population of isolated CD45⁺ cells.
Embodiment 307. The method of any one of embodiments 256-306, wherein third population of isolated CD45⁺ cells is administered approximately 12 hours, approximately 15 hours, approximately 18 hours, approximately 21 hours, approximately, 24 hours, approximately 27 hours, approximately 30 hours, approximately 33 hours, approximately 36 hours, approximately 39 hours, approximately 42 hours, approximately 45 hours, approximately 48 hours, approximately 51 hours, approximately 54 hours, approximately 57 hours, approximately 60 hours, approximately 63 hours, approximately 66 hours, approximately 69 hours, approximately 72 hours, approximately 75 hours, approximately 78 hours, approximately 81 hours, approximately 84 hours, approximately 87 hours, approximately 90 hours, approximately 93 hours, approximately 96 hours, approximately 99 hours, approximately 102 hours, approximately 105 hours, approximately 108 hours, approximately 111 hours, approximately 114 hours, approximately 117 hours, approximately 120 hours after administration of the second population of isolated CD45⁺ cells.
Embodiment 308. A method of treating a human subject having or suspected of having a hematologic malignancy with a multi-component cellular therapy, the method comprising:
a) administering a first population of isolated CD45⁺ cells comprising CD34⁺ hematopoietic stem and progenitor cells (HSPCs);
b) following administration of the first population of isolated CD45⁺ cells, administering a second population of isolated CD45⁺ cells comprising isolated fresh CD4⁺CD25⁺CD127^{dim} regulatory T cells (Tregs); and
c) administering a third population of isolated CD45⁺ cells comprising conventional CD3⁺ T cells (Tcons) from approximately 12 hours to approximately 120 hours after administration of the second population of isolated CD45⁺ cells,
wherein the multi-component cellular therapy comprises the first population of isolated CD45⁺ cells, the second population of isolated CD45⁺ cells, and the third population of isolated CD45⁺ cells.
Embodiment 309. The method of embodiment 308, wherein the multi-component cellular therapy comprises the multi-component cellular therapy product of any one of embodiments 1-51.
Embodiment 310. The method of embodiment 308, wherein the multi-component cellular therapy comprises the multi-component pharmaceutical treatment of any one of embodiments 109-138 or 181-193.
Embodiment 311. The method of any one of embodiments 308-310, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN).
Embodiment 312. The method of any one of embodiments 308-311, wherein the second population of CD45⁺ cells is administered from approximately 5 minutes to approximately 5 hours after administration of the first population of CD45⁺ cells.
Embodiment 313. The method of any one of embodiments 308-312, wherein the second population of CD45⁺ cells is administered approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 1 hour, approximately 1.10 hours, approximately 1.20 hours, approximately 1.30 hours, approximately 1.40 hours, approximately 1.50 hours, approximately 1.60 hours, approximately 1.70 hours, approximately 1.80 hours, approximately 1.90 hours, approximately 2 hours, approximately 2.10 hours, approximately 2.20 hours, approximately 2.30 hours, approximately 2.40 hours, approximately 2.50 hours, approximately 2.60 hours, approximately 2.70 hours, approximately 2.80 hours, approximately 2.90 hours, approximately 3 hours, approximately 3.10 hours, approximately 3.30 hours, approximately 3.30 hours, approximately 3.40 hours, approximately 3.50 hours, approximately 3.60 hours, approximately 3.70 hours, approximately 3.80 hours, approximately 3.90 hours, approximately 4 hours, approximately 4.10 hours, approximately 4.40 hours, approximately 4.40 hours, approximately 4.40 hours, approximately 4.50 hours, approximately 4.60 hours, approximately 4.70 hours, approximately 4.80 hours, approximately 4.90 hours, or approximately 5 hours after administration of the first population of CD45⁺ cells.
Embodiment 314. The method of any one of embodiments 308-313, wherein third population of isolated CD45⁺ cells is administered from approximately 12 hours to approximately 120 hours after administration of the first population of isolated CD45⁺ cells.
Embodiment 315. The method of any one of embodiments 308-314, wherein third population of isolated CD45⁺ cells is administered approximately 12 hours, approximately 15 hours, approximately 18 hours, approximately 21 hours, approximately, 24 hours, approximately 27 hours, approximately 30 hours, approximately 33 hours, approximately 36 hours, approximately 39 hours, approximately 42 hours, approximately 45 hours, approximately 48 hours, approximately 51 hours, approximately 54 hours, approximately 57 hours, approximately 60 hours, approximately 63 hours, approximately 66 hours, approximately 69 hours, approximately 72 hours, approximately 75 hours, approximately 78 hours, approximately 81 hours, approximately 84 hours, approximately 87 hours, approximately 90 hours, approximately 93 hours, approximately 96 hours, approximately 99 hours, approximately 102 hours, approximately 105 hours, approximately 108 hours, approximately 111 hours, approximately 114 hours, approximately 117 hours, approximately 120 hours after administration of the first population of isolated CD45⁺ cells.
Embodiment 316. The method of any one of embodiments 308-315, wherein third population of isolated CD45⁺ cells is administered approximately 12 hours, approximately 15 hours, approximately 18 hours, approximately 21 hours, approximately, 24 hours, approximately 27 hours, approximately 30 hours, approximately 33 hours, approximately 36 hours, approximately 39 hours, approximately 42 hours, approximately 45 hours, approximately 48 hours, approximately 51 hours, approximately 54 hours, approximately 57 hours, approximately 60 hours, approximately 63 hours, approximately 66 hours, approximately 69 hours, approximately 72 hours, approximately 75 hours, approximately 78 hours, approximately 81 hours, approximately 84 hours, approximately 87 hours, approximately 90 hours, approximately 93 hours, approximately 96 hours, approximately 99 hours, approximately 102 hours, approximately 105 hours, approximately 108 hours, approximately 111 hours, approximately 114 hours, approximately 117 hours, approximately 120 hours after administration of the second population of isolated CD45⁺ cells.
Embodiment 317. The method of any one of embodiments 256-316, wherein the multi-component cellular therapy further comprises a solution comprising one or more doses of a graft vs host disease (GVHD) prophylactic agent.
Embodiment 318. The method of embodiment 317, wherein the GVHS prophylactic agent is tacrolimus.
Embodiment 319. The method of embodiment 318, wherein the tacrolimus is provided in an amount sufficient to maintain a trough blood level of approximately 5 ng/mL to approximately 10 ng/mL in the human subject.
Embodiment 320. The method of embodiment 318 or embodiment 319, wherein the tacrolimus is provided at a dose that ranges from approximately 0.01 mg per kilogram of body weight of the human subject to 0.50 mg per kilogram of body weight of the human subject twice per day.
Embodiment 321. The method of any one of embodiments 318-320, wherein the tacrolimus is dosed in an amount to maintain or that maintains a target blood level of approximately 1 ng/ml to approximately 10 ng/mL in the human subject for approximately 20 days or more, approximately 25 days or more, approximately 30 days or more, approximately 35 days or more, approximately 40 days or more, approximately 55 days or more, approximately 50 days or more, approximately 55 days or more, approximately 60 days or more, approximately 65 days or more, approximately 770 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the third population of CD45⁺ cells.
Embodiment 322. The method of any one of embodiments 318-321, wherein the tacrolimus is initially administered from approximately 12 hours to approximately 24 hours after the third population of isolated CD45⁺ cells.
Embodiment 323. The method of any one of embodiments 318-322, wherein administration of the tacrolimus is tapered starting at approximately 90 days after initial administration of the tacrolimus.
Embodiment 324. The method of any one of embodiments 256-323, wherein the administering comprises infusing into the human subject the first population of isolated CD45⁺ cells, the second population of isolated CD45⁺ cells, and the third population of isolated CD45⁺ cells.
Embodiment 325. The method of any one of embodiments 256-324, wherein the first population of isolated CD45⁺ cells comprises from approximately 1.0×10⁵ to approximately 1.0×10⁸ HSPCs per kilogram of body weight of the human subject, from approximately 5.0×10⁵ to approximately 5.0×10⁸ HSPCs, or from approximately 1.5×10⁷ to approximately 1.5×10¹⁰ HSPCs.
Embodiment 326. The method of any one of embodiments 256-325, wherein the first population of isolated CD45⁺ cells comprises approximately 1.0×10⁵ or more HSPCs per kilogram of body weight of the human subject, approximately 2.0×10⁵ or more HSPCs per kilogram of body weight of the human subject, approximately 3.0×10⁵ or more HSPCs per kilogram of body weight of the human subject, approximately 4.0×10⁵ or more HSPCs per kilogram of body weight of the human subject, approximately 5.0×10⁵ or more HSPCs per kilogram of body weight of the human subject, approximately 6.0×10⁵ or more HSPCs per kilogram of body weight of the human subject, approximately 7.0×10⁵ or more HSPCs per kilogram of body weight of the human subject, approximately 8.0×10⁵ or more HSPCs per kilogram of body weight of the human subject, approximately 9.0×10⁵ or more HSPCs per kilogram of body weight of the human subject, approximately 1.0×10⁶ or more HSPCs per kilogram of body weight of the human subject, approximately 1.5×10⁶ or more HSPCs per kilogram of body weight of the human subject, approximately 2.0×10⁶ or more HSPCs per kilogram of body weight of the human subject, approximately 2.5×10⁶ or more HSPCs per kilogram of body weight of the human subject, approximately 3.0×10⁶ or more HSPCs per kilogram of body weight of the human subject, approximately 3.5×10⁶ or more HSPCs per kilogram of body weight of the human subject, approximately 4.0×10⁶ or more HSPCs per kilogram of body weight of the human subject, approximately 4.5×10⁶ or more HSPCs per kilogram of body weight of the human subject, approximately 5.0×10⁶ or more HSPCs per kilogram of body weight of the human subject, approximately 5.5×10⁶ or more HSPCs per kilogram of body weight of the human subject, approximately 6.0×10⁶ or more HSPCs per kilogram of body weight of the human subject, approximately 6.5×10⁶ or more HSPCs per kilogram of body weight of the human subject, approximately 7.0×10⁶ or more HSPCs per kilogram of body weight of the human subject, approximately 7.5×10⁶ or more HSPCs per kilogram of body weight of the human subject, approximately 8.0×10⁶ or more HSPCs per kilogram of body weight of the human subject, approximately 8.5×10⁶ or more HSPCs per kilogram of body weight of the human subject, approximately 9.0×10⁶ or more HSPCs per kilogram of body weight of the human subject, approximately 9.5×10⁶ or more HSPCs per kilogram of body weight of the human subject, approximately 1.0×10⁷ or more HSPCs per kilogram of body weight of the human subject, approximately 1.5×10⁷ or more HSPCs per kilogram of body weight of the human subject, approximately 2.0×10⁷ or more HSPCs per kilogram of body weight of the human subject, approximately 2.5×10⁷ or more HSPCs per kilogram of body weight of the human subject, approximately 3.0×10⁷ or more HSPCs per kilogram of body weight of the human subject, approximately 3.5×10⁷ or more HSPCs per kilogram of body weight of the human subject, approximately 4.0×10⁷ or more HSPCs per kilogram of body weight of the human subject, approximately 4.5×10⁷ or more HSPCs per kilogram of body weight of the human subject, approximately 5.0×10⁷ or more HSPCs per kilogram of body weight of the human subject, approximately 5.5×10⁷ or more HSPCs per kilogram of body weight of the human subject, approximately 6.0×10⁷ or more HSPCs per kilogram of body weight of the human subject, approximately 6.5×10⁷ or more HSPCs per kilogram of body weight of the human subject, approximately 7.0×10⁷ or more HSPCs per kilogram of body weight of the human subject, approximately 7.5×10⁷ or more HSPCs per kilogram of body weight of the human subject, approximately 8.0×10⁷ or more HSPCs per kilogram of body weight of the human subject, approximately 8.5×10⁷ or more HSPCs per kilogram of body weight of the human subject, approximately 9.0×10⁷ or more HSPCs per kilogram of body weight of the human subject, approximately 9.5×10⁷ or more HSPCs per kilogram of body weight of the human subject, or approximately 1.0×10⁸ or more HSPCs per kilogram of body weight of the human subject.
Embodiment 327. The method of any one of embodiments 256-326, wherein the first population of isolated CD45⁺ cells comprises approximately 5.0×10⁵ or more HSPCs, approximately 6.0×10⁵ or more HSPCs, approximately 7.0×10⁵ or more HSPCs, approximately 8.0×10⁵ or more HSPCs, approximately 9.0×10⁵ or more HSPCs, approximately 1.0×10⁶ or more HSPCs, approximately 1.5×10⁶ or more HSPCs, approximately 2.0×10⁶ or more HSPCs, approximately 2.5×10⁶ or more HSPCs, approximately 3.0×10⁶ or more HSPCs, approximately 3.5×10⁶ or more HSPCs, approximately 4.0×10⁶ or more HSPCs, approximately 4.5×10⁶ or more HSPCs, approximately 5.0×10⁶ or more HSPCs, approximately 5.5×10⁶ or more HSPCs, approximately 6.0×10⁶ or more HSPCs, approximately 6.5×10⁶ or more HSPCs, approximately 7.0×10⁶ or more HSPCs, approximately 7.5×10⁶ or more HSPCs, approximately 8.0×10⁶ or more HSPCs, approximately 8.5×10⁶ or more HSPCs, approximately 9.0×10⁶ or more HSPCs, approximately 9.5×10⁶ or more HSPCs, approximately 1.0×10⁷ or more HSPCs, approximately 1.5×10⁷ or more HSPCs, approximately 2.0×10⁷ or more HSPCs, approximately 2.5×10⁷ or more HSPCs, approximately 3.0×10⁷ or more HSPCs, approximately 3.5×10⁷ or more HSPCs, approximately 4.0×10⁷ or more HSPCs, approximately 4.5×10⁷ or more HSPCs, approximately 5.0×10⁷ or more HSPCs, approximately 5.5×10⁷ or more HSPCs, approximately 6.0×10⁷ or more HSPCs, approximately 6.5×10⁷ or more HSPCs, approximately 7.0×10⁷ or more HSPCs, approximately 7.5×10⁷ or more HSPCs, approximately 8.0×10⁷ or more HSPCs, approximately 8.5×10⁷ or more HSPCs, approximately 9.0×10⁷ or more HSPCs, approximately 9.5×10⁷ or more HSPCs, approximately 1.0×10⁸ or more HSPCs, approximately 1.5×10⁸ or more HSPCs, approximately 2.0×10⁸ or more HSPCs, approximately 2.5×10⁸ or more HSPCs, approximately 3.0×10⁸ or more HSPCs, approximately 3.5×10⁸ or more HSPCs, approximately 4.0×10⁸ or more HSPCs, approximately 4.5×10⁸ or more HSPCs, approximately 5.0×10⁸ or more HSPCs, approximately 5.5×10⁸ or more HSPCs, approximately 6.0×10⁸ or more HSPCs, approximately 6.5×10⁸ or more HSPCs, approximately 7.0×10⁸ or more HSPCs, approximately 7.5×10⁸ or more HSPCs, approximately 8.0×10⁸ or more HSPCs, approximately 8.5×10⁸ or more HSPCs, approximately 9.0×10⁸ or more HSPCs, approximately 9.5×10⁸ or more HSPCs, approximately 1.0×10⁹ or more HSPCs, approximately 1.5×10⁹ or more HSPCs, approximately 2.0×10⁹ or more HSPCs, approximately 2.5×10⁹ or more HSPCs, approximately 3.0×10⁹ or more HSPCs, approximately 3.5×10⁹ or more HSPCs, approximately 4.0×10⁹ or more HSPCs, approximately 4.5×10⁹ or more HSPCs, approximately 5.0×10⁹ or more HSPCs, approximately 5.5×10⁹ or more HSPCs, approximately 6.0×10⁹ or more HSPCs, approximately 6.5×10⁹ or more HSPCs, approximately 7.0×10⁹ or more HSPCs, approximately 7.5×10⁹ or more HSPCs, approximately 8.0×10⁹ or more HSPCs, approximately 8.5×10⁹ or more HSPCs, approximately 9.0×10⁹ or more HSPCs, approximately 9.5×10⁹ or more HSPCs, approximately 1.0×10¹⁰ or more HSPCs, or approximately 1.5×10¹⁰ or more HSPCs.
Embodiment 328. The method of any one of embodiments 256-327, wherein the Tregs are FOXP3⁺.
Embodiment 329. The method of any one of embodiments 256-328, wherein in the second population of isolated CD45⁺ cells more than approximately 90% of the CD45⁺ cells are Tregs.
Embodiment 330. The method of any one of embodiments 256-3229, wherein the second population of isolated CD45⁺ cells comprises from approximately 1.0×10⁵ to approximately 2.0×10⁷ Tregs per kilogram of body weight of the human subject, from approximately 1.5×10⁷ to approximately 3.0×10⁹ Tregs, or from approximately 5.0×10⁵ to approximately 1.0×10⁸ Tregs.
Embodiment 331. The method of any one of embodiments 256-330, wherein the second population of isolated CD45⁺ cells comprises approximately 1.0×10⁵ or more Tregs per kilogram of body weight of the human subject, approximately 2.0×10⁵ or more Tregs per kilogram of body weight of the human subject, approximately 3.0×10⁵ or more Tregs per kilogram of body weight of the human subject, approximately 4.0×10⁵ or more Tregs per kilogram of body weight of the human subject, approximately 5.0×10⁵ or more Tregs per kilogram of body weight of the human subject, approximately 6.0×10⁵ or more Tregs per kilogram of body weight of the human subject, approximately 7.0×10⁵ or more Tregs per kilogram of body weight of the human subject, approximately 8.0×10⁵ or more Tregs per kilogram of body weight of the human subject, approximately 9.0×10⁵ or more Tregs per kilogram of body weight of the human subject, approximately 1.0×10⁶ or more Tregs per kilogram of body weight of the human subject, approximately 1.5×10⁶ or more Tregs per kilogram of body weight of the human subject, approximately 2.0×10⁶ or more Tregs per kilogram of body weight of the human subject, approximately 2.5×10⁶ or more Tregs per kilogram of body weight of the human subject, approximately 3.0×10⁶ or more Tregs per kilogram of body weight of the human subject, approximately 3.5×10⁶ or more Tregs per kilogram of body weight of the human subject, approximately 4.0×10⁶ or more Tregs per kilogram of body weight of the human subject, approximately 4.5×10⁶ or more Tregs per kilogram of body weight of the human subject, approximately 5.0×10⁶ or more Tregs per kilogram of body weight of the human subject, approximately 5.5×10⁶ or more Tregs per kilogram of body weight of the human subject, approximately 6.0×10⁶ or more Tregs per kilogram of body weight of the human subject, approximately 6.5×10⁶ or more Tregs per kilogram of body weight of the human subject, approximately 7.0×10⁶ or more Tregs per kilogram of body weight of the human subject, approximately 7.5×10⁶ or more Tregs per kilogram of body weight of the human subject, approximately 8.0×10⁶ or more Tregs per kilogram of body weight of the human subject, approximately 8.5×10⁶ or more Tregs per kilogram of body weight of the human subject, approximately 9.0×10⁶ or more Tregs per kilogram of body weight of the human subject, approximately 9.5×10⁶ or more Tregs per kilogram of body weight of the human subject, approximately 1.0×10⁷ or more Tregs per kilogram of body weight of the human subject, approximately 1.5×10⁷ or more Tregs per kilogram of body weight of the human subject, or approximately 2.0×10⁷ or more Tregs per kilogram of body weight of the human subject.
Embodiment 332. The method of any one of embodiments 256-331, wherein the second population of isolated CD45⁺ cells comprises approximately 5.0×10⁵ or more Tregs, approximately 6.0×10⁵ or more Tregs, approximately 7.0×10⁵ or more Tregs, approximately 8.0×10⁵ or more Tregs, approximately 9.0×10⁵ or more Tregs, approximately 1.0×10⁶ or more Tregs, approximately 1.5×10⁶ or more Tregs, approximately 2.0×10⁶ or more Tregs, approximately 2.5×10⁶ or more Tregs, approximately 3.0×10⁶ or more Tregs, approximately 3.5×10⁶ or more Tregs, approximately 4.0×10⁶ or more Tregs, approximately 4.5×10⁶ or more Tregs, approximately 5.0×10⁶ or more Tregs, approximately 5.5×10⁶ or more Tregs, approximately 6.0×10⁶ or more Tregs, approximately 6.5×10⁶ or more Tregs, approximately 7.0×10⁶ or more Tregs, approximately 7.5×10⁶ or more Tregs, approximately 8.0×10⁶ or more Tregs, approximately 8.5×10⁶ or more Tregs, approximately 9.0×10⁶ or more Tregs, approximately 9.5×10⁶ or more Tregs, approximately 1.0×10⁷ or more Tregs, approximately 1.5×10⁷ or more Tregs, approximately 2.0×10⁷ or more Tregs, approximately 2.5×10⁷ or more Tregs, approximately 3.0×10⁷ or more Tregs, approximately 3.5×10⁷ or more Tregs, approximately 4.0×10⁷ or more Tregs, approximately 4.5×10⁷ or more Tregs, approximately 5.0×10⁷ or more Tregs, approximately 5.5×10⁷ or more Tregs, approximately 6.0×10⁷ or more Tregs, approximately 6.5×10⁷ or more Tregs, approximately 7.0×10⁷ or more Tregs, approximately 7.5×10⁷ or more Tregs, approximately 8.0×10⁷ or more Tregs, approximately 8.5×10⁷ or more Tregs, approximately 9.0×10⁷ or more Tregs, approximately 9.5×10⁷ or more Tregs, approximately 1.0×10⁸ or more Tregs, approximately 1.5×10⁸ or more Tregs, approximately 2.0×10⁸ or more Tregs, approximately 2.5×10⁸ or more Tregs, approximately 3.0×10⁸ or more Tregs, approximately 3.5×10⁸ or more Tregs, approximately 4.0×10⁸ or more Tregs, approximately 4.5×10⁸ or more Tregs, approximately 5.0×10⁸ or more Tregs, approximately 5.5×10⁸ or more Tregs, approximately 6.0×10⁸ or more Tregs, approximately 6.5×10⁸ or more Tregs, approximately 7.0×10⁸ or more Tregs, approximately 7.5×10⁸ or more Tregs, approximately 8.0×10⁸ or more Tregs, approximately 8.5×10⁸ or more Tregs, approximately 9.0×10⁸ or more Tregs, approximately 9.5×10⁸ or more Tregs, approximately 1.0×10⁹ or more Tregs, approximately 1.5×10⁹ or more Tregs, approximately 2.0×10⁹ or more Tregs, approximately 2.5×10⁹ or more Tregs, or approximately 3.0×10⁹ or more Tregs.
Embodiment 333. The method of any one of embodiments 256-332, wherein the third population of isolated CD45⁺ cells comprises from approximately 1.0×10⁵ to approximately 4.0×107 Tcons per kilogram of body weight of the human subject, from approximately 5.0×10⁵ to approximately 2.0×10⁸ Tcons, or from approximately 1.5×10⁷ to approximately 6.0×10⁹ Tcons.
Embodiment 334. The method of any one of embodiments 256-333, wherein the third population of isolated CD45⁺ cells comprises approximately 1.0×10⁵ or more Tcons per kilogram of body weight of the human subject, approximately 2.0×10⁵ or more Tcons per kilogram of body weight of the human subject, approximately 3.0×10⁵ or more Tcons per kilogram of body weight of the human subject, approximately 4.0×10⁵ or more Tcons per kilogram of body weight of the human subject, approximately 5.0×10⁵ or more Tcons per kilogram of body weight of the human subject, approximately 6.0×10⁵ or more Tcons per kilogram of body weight of the human subject, approximately 7.0×10⁵ or more Tcons per kilogram of body weight of the human subject, approximately 8.0×10⁵ or more Tcons per kilogram of body weight of the human subject, approximately 9.0×10⁵ or more Tcons per kilogram of body weight of the human subject, approximately 1.0×10⁶ or more Tcons per kilogram of body weight of the human subject, approximately 1.5×10⁶ or more Tcons per kilogram of body weight of the human subject, approximately 2.0×10⁶ or more Tcons per kilogram of body weight of the human subject, approximately 2.5×10⁶ or more Tcons per kilogram of body weight of the human subject, approximately 3.0×10⁶ or more Tcons per kilogram of body weight of the human subject, approximately 3.5×10⁶ or more Tcons per kilogram of body weight of the human subject, approximately 4.0×10⁶ or more Tcons per kilogram of body weight of the human subject, approximately 4.5×10⁶ or more Tcons per kilogram of body weight of the human subject, approximately 5.0×10⁶ or more Tcons per kilogram of body weight of the human subject, approximately 5.5×10⁶ or more Tcons per kilogram of body weight of the human subject, approximately 6.0×10⁶ or more Tcons per kilogram of body weight of the human subject, approximately 6.5×10⁶ or more Tcons per kilogram of body weight of the human subject, approximately 7.0×10⁶ or more Tcons per kilogram of body weight of the human subject, approximately 7.5×10⁶ or more Tcons per kilogram of body weight of the human subject, approximately 8.0×10⁶ or more Tcons per kilogram of body weight of the human subject, approximately 8.5×10⁶ or more Tcons per kilogram of body weight of the human subject, approximately 9.0×10⁶ or Tcons per kilogram of body weight of the human subject, approximately 9.5×10⁶ or more Tcons per kilogram of body weight of the human subject, approximately 1.0×10⁷ or more Tcons per kilogram of body weight of the human subject, approximately 1.5×10⁷ or more Tcons per kilogram of body weight of the human subject, approximately 2.0×10⁷ or more Tcons per kilogram of body weight of the human subject, approximately 2.5×10⁷ or more Tcons per kilogram of body weight of the human subject, approximately 3.0×10⁷ or more Tcons per kilogram of body weight of the human subject, approximately 3.5×10⁷ or more Tcons per kilogram of body weight of the human subject, or approximately 4.0×10⁷ or more Tcons per kilogram of body weight of the human subject.
Embodiment 335. The method of any one of embodiments 256-334, wherein the third population of isolated CD45⁺ cells comprises approximately 5.0×10⁵ or more Tcons, approximately 6.0×10⁵ or more Tcons, approximately 7.0×10⁵ or more Tcons, approximately 8.0×10⁵ or more Tcons, approximately 9.0×10⁵ or more Tcons, approximately 1.0×10⁶ or more Tcons, approximately 1.5×10⁶ or more Tcons, approximately 2.0×10⁶ or more Tcons, approximately 2.5×10⁶ or more Tcons, approximately 3.0×10⁶ or more Tcons, approximately 3.5×10⁶ or more Tcons, approximately 4.0×10⁶ or more Tcons, approximately 4.5×10⁶ or more Tcons, approximately 5.0×10⁶ or more Tcons, approximately 5.5×10⁶ or more Tcons, approximately 6.0×10⁶ or more Tcons, approximately 6.5×10⁶ or more Tcons, approximately 7.0×10⁶ or more Tcons, approximately 7.5×10⁶ or more Tcons, approximately 8.0×10⁶ or more Tcons, approximately 8.5×10⁶ or more Tcons, approximately 9.0×10⁶ or Tcons, approximately 9.5×10⁶ or more Tcons, approximately 1.0×10⁷ or more Tcons, approximately 1.5×10⁷ or more Tcons, approximately 2.0×10⁷ or more Tcons, approximately 2.5×10⁷ or more Tcons, approximately 3.0×10⁷ or more Tcons, approximately 3.5×10⁷ or more Tcons, approximately 4.0×10⁷ or more Tcons, approximately 4.5×10⁷ or more Tcons, approximately 5.0×10⁷ or more Tcons, approximately 5.5×10⁷ or more Tcons, approximately 6.0×10⁷ or more Tcons, approximately 6.5×10⁷ or more Tcons, approximately 7.0×10⁷ or more Tcons, approximately 7.5×10⁷ or more Tcons, approximately 8.0×10⁷ or more Tcons, approximately 8.5×10⁷ or more Tcons, approximately 9.0×10⁷ or more Tcons, approximately 9.5×10⁷ or more Tcons, approximately 1.0×10⁸ or more Tcons, approximately 1.5×10⁸ or more Tcons, approximately 2.0×10⁸ or more Tcons, approximately 2.5×10⁸ or more Tcons, approximately 3.0×10⁸ or more Tcons, approximately 3.5×10⁸ or more Tcons, approximately 4.0×10⁸ or more Tcons, approximately 4.5×10⁸ or more Tcons, approximately 5.0×10⁸ or more Tcons, approximately 5.5×10⁸ or more Tcons, approximately 6.0×10⁸ or more Tcons, approximately 6.5×10⁸ or more Tcons, approximately 7.0×10⁸ or more Tcons, approximately 7.5×10⁸ or more Tcons, approximately 8.0×10⁸ or more Tcons, approximately 8.5×10⁸ or more Tcons, approximately 9.0×10⁸ or more Tcons, approximately 9.5×10⁸ or more Tcons, approximately 1.0×10⁹ or more Tcons, approximately 1.5×10⁹ or more Tcons, approximately 2.0×10⁹ or more Tcons, approximately 2.5×10⁹ or more Tcons, approximately 3.0×10⁹ or more Tcons, approximately 3.5×10⁹ or more Tcons, approximately 4.0×10⁹ or more Tcons, approximately 4.5×10⁹ or more Tcons, approximately 5.0×10⁹ or more Tcons, approximately 5.5×10⁹ or more Tcons, or approximately 6.0×10⁹ or more Tcons.
Embodiment 336. The method of any one of embodiments 256-335, wherein the human subject does not develop higher than stage 2 GVHD within approximately 100 days of the administering of the third population of isolated CD45⁺ cells.
Embodiment 337. The method of any one of embodiments 256-336, wherein the human subject does not develop higher than stage 2 GVHD within approximately 180 days or within approximately 200 days of the administering of the third population of isolated CD45⁺ cells.
Embodiment 338. The method of any one of embodiments 256-337, wherein the human subject does not develop higher than stage 2 GVHD within approximately 1 year of the administering of the third population of isolated CD45⁺ cells.
Embodiment 339. The method of any one of embodiments 256-338, wherein the method further comprises administering approximately 1000 mg of mycophenolate mofetil (MMF).
Embodiment 340. The method of embodiment 339, wherein the MMF is initially administered from approximately 12 hours to approximately 24 hours after the administering of the third population of isolated CD45⁺ cells.
Embodiment 341. The method of embodiment 339 or embodiment 340, wherein administration of the MMF is tapered starting at approximately 30 days, at approximately 35 days, at approximately 40 days, at approximately 41 days, at approximately 42 days, at approximately 43 days, at approximately 44 days, at approximately 45 days, at approximately 46 days, at approximately 47 days, at approximately 48 days, at approximately 49 days, or at approximately 50 days after initial administration of the MMF.
Embodiment 342. The method of any one of embodiments 256-341, wherein the first population of isolated CD45⁺ cells, the second population of isolated CD45⁺ cells, and the third population of isolated CD45⁺ cells are from an allogeneic donor having at least one HLA mismatch relative to the human subject.
Embodiment 343. The method of embodiment 342, wherein the HLA-mismatched donor is unrelated to the human subject.
Embodiment 344. The method of embodiment 342, wherein the HLA-mismatched donor is related to the human subject.
Embodiment 345. The method of any one of embodiments 342-344, wherein the at least one HLA mismatch is at an allele selected from the group consisting of: HLA-A, HLA-B, HLA-C, HLA-DRB1, and any combination thereof.
Embodiment 346. The method of any one of embodiments 342-345, wherein the donor that has at least one HLA mismatch is 6/8 HLA-mismatched relative to the human subject or is 7/8 HLA-mismatched relative to the human subject.
Embodiment 347. The method of embodiment 346, wherein the donor is 7/8 HLA-mismatched relative to the human subject.
Embodiment 348. The method of embodiment 347, wherein the donor that is 7/8 HLA-mismatched relative to the human subject has a mismatch in HLA-A.
Embodiment 349. The method of embodiment 347, wherein the donor that is 7/8 HLA-mismatched relative to the human subject has a mismatch in HLA-B.
Embodiment 350. The method of embodiment 347, wherein the donor that is 7/8 HLA-mismatched relative to the human subject has a mismatch in HLA-C.
Embodiment 351. The method of embodiment 347, wherein the donor that is 7/8 HLA-mismatched relative to the human subject has a mismatch in HLA-DRB1.
Embodiment 352. The method of any one of embodiments 342-351, wherein the donor that has at least one HLA mismatch relative to the human subject has a mismatched HLA allele as a result of the donor being homozygous for the HLA allele while the human subject is heterogeneous for the HLA allele.
Embodiment 353. The method of any one of embodiments 342-352, wherein the donor that has at least one HLA mismatch relative to the human subject has a mismatched HLA allele as a result of the donor being heterozygous for the HLA allele while the human subject is homozygous for the HLA allele.
Embodiment 354. The method of any one of embodiments 342-353, wherein the donor that has at least one HLA mismatch relative to the human subject has a mismatched HLA allele as a result of both the donor and the human subject being heterozygous for the HLA allele.
Embodiment 355. The method of any one of embodiments 342-354, wherein incidence of non-relapse mortality of the human subject is decreased after administration of the multi-component cellular therapy, relative to a corresponding human subject that has been administered a standard myeloablative allogeneic hematopoietic stem cell transplant (alloHSCT) from a donor that has at least one HLA mismatch relative to the corresponding human subject.
Embodiment 356. The method of any one of embodiments 342-355, wherein overall survival of the human subject is increased after administration of the multi-component cellular therapy, relative to a corresponding human subject that has been administered a standard myeloablative allogeneic hematopoietic stem cell transplant (alloHSCT) from a donor that has at least one HLA mismatch relative to the corresponding human subject.
Embodiment 357. The method of any one of embodiments 256-356, wherein the method further comprises collecting one or more, or two or more mobilized peripheral blood donations from the donor.
Embodiment 358. The method of any one of embodiments 256-357, wherein the method further comprises collecting at most two mobilized peripheral blood donations from the donor.
Embodiment 359. The method of embodiment 83 or embodiment 358, wherein the peripheral blood donations are mobilized by granulocyte colony-stimulating factor (G-CSF), granulocyte macrophage colony-stimulating factor (GM-CSF), plerixafor, or any combination thereof.
Embodiment 360. The method of any one of embodiments 357-359, wherein at least one of the mobilized peripheral blood donations is processed and sorted using one or more immune-separation particles (ISPs) to enrich CD34⁺ cells and Tregs.
Embodiment 361. The method of embodiment 360, wherein the one or more ISPs comprise affinity reagents, optionally wherein the affinity reagents are immuno-magnetic separation particles, optionally wherein the immuno-magnetic separation particles are antibodies each conjugated to an iron-containing particle.
Embodiment 362. The method of embodiment 360 or embodiment 361, wherein an average number of ISP's per HSPC in the first population of isolated CD45⁺ cells is equal to or less than approximately 20,000.
Embodiment 363. The method of embodiment 360 or embodiment 361, wherein an average number of ISP's per HSPC in the first population of isolated CD45⁺ cells is from approximately 1000 to approximately 20,000.
Embodiment 364. The method of any one of embodiments 361-363, wherein the affinity reagents comprise a plurality of CD25-reagents that binds to one or more CD25 receptors on a Treg.
Embodiment 365. The method of embodiment 364, wherein an average number of ISPs per Treg in the second population of isolated CD45⁺ cells is equal or less than approximately 4000.
Embodiment 366. The method of embodiment 364, wherein an average number of ISPs per Treg in the second population of isolated CD45⁺ cells is from approximately 1500 to approximately 2500.
Embodiment 367. The method of any one of embodiments 256-366, wherein the method further comprises a conditioning regimen, wherein the conditioning regimen is administered before administration of the multi-component cellular therapy.
Embodiment 368. The method of embodiment 367, wherein the conditioning regimen is administered from approximately two days to approximately ten days before administration of the multi-component cellular therapy.
Embodiment 369. The method of embodiment 367 or embodiment 368, wherein the conditioning regimen is a total body irradiation-based (TBI-based) regimen.
Embodiment 370. The method of embodiment 369, wherein the TBI-based regimen further comprises one or more conditioning reagents.
Embodiment 371. The method of embodiment 370, wherein the one or more conditioning reagents are selected from the group consisting of: cyclophosphamide, etoposide, thiotepa, and any combination thereof.
Embodiment 372. The method of embodiment 367 or embodiment 368, wherein the conditioning regimen is a myeloablative conditioning regimen.
Embodiment 373. The method of embodiment 372, wherein the myeloablative conditioning regimen comprises one or more conditioning reagents.
Embodiment 374. The method of embodiment 373, wherein the one or more conditioning reagents are selected from the group consisting of: thiotepa, busulfan, melphalan, fludarabine, cyclophosphamide, anti-thymocyte globulin (ATG), and any combination thereof.
Embodiment 375. The method of embodiment 373, wherein the myeloablative conditioning regimen comprises three or more conditioning reagents, wherein at least one conditioning reagent comprises thiotepa.
Embodiment 376. The method of any one of embodiments 373-375, wherein the myeloablative conditioning regimen comprises one or more doses of busulfan, fludarabine, and thiotepa.
Embodiment 377. The method of embodiment 376, wherein the one or more doses of busulfan, fludarabine, and thiotepa comprise from approximately 5 to approximately 12 mg of thiotepa per kilogram of body weight of the human subject, from approximately 7 to approximately 11 mg of busulfan per kilogram of body weight of the human subject, and from approximately 100 to
Embodiment 378. The method of any one of embodiments 367-377, wherein GVHD and relapse-free survival (GFRS) of the human subject increases by at least approximately 3.5-fold after administration of the multi-component cellular therapy, relative to a corresponding human subject that has been administered a standard myeloablative allogeneic hematopoietic stem cell transplant (alloHSCT).
Embodiment 379. The method of any one of embodiments 367-378, wherein overall survival increases by at least approximately 1.25-fold after administration of the multi-component cellular therapy, relative to a corresponding human subject that has been administered a standard myeloablative allogeneic hematopoietic stem cell transplant (alloHSCT).
Embodiment 380. The method of any one of embodiments 367-379, wherein incidence of non-relapse mortality decreases by at least approximately 60% after administration of the multi-component cellular therapy, relative to a corresponding human subject that has been administered a standard myeloablative allogeneic hematopoietic stem cell transplant (alloHSCT).
Embodiment 381. The method of any one of embodiments 256-380, wherein the human subject is approximately 3 months of age or older.
Embodiment 382. The method of any one of embodiments 256-381, wherein the human subject is from between approximately 3 months to approximately 18 years of age.
Embodiment 383. The method of any one of embodiments 256-380, wherein the human subject is approximately 18 years of age or older.
Embodiment 384. The method of embodiment 383, wherein the human subject is between approximately 18 years to approximately 65 years of age.
Embodiment 385. The method of embodiment 383, wherein the human subject is between approximately 18 years to approximately 75 years of age.
Embodiment 386. The method of embodiment 385, wherein the human subject is from approximately 66 years of age to approximately 75 years of age.
Embodiment 387. The method of any one of embodiments 256-380, wherein the human subject is from between approximately 3 months to approximately 75 years of age.
Embodiment 388. The method of any one of embodiments 256-387, wherein the human subject has received from one to five previous lines of therapy.
Embodiment 389. The method of any one of embodiments 256-388, wherein chronic GVHD-free survival increases after administration of the multi-component cellular therapy, relative to a corresponding human subject that has been administered a standard myeloablative allogeneic hematopoietic stem cell transplant (alloHSCT).
Embodiment 390. The method of embodiment 389, wherein the human subject experiences chronic GVHD-free survival for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 391. The method of any one of embodiments 256-390, wherein incidence of primary graft failure or secondary graft failure decreases after administration of the multi-component cellular therapy, relative to a corresponding human subject that has been administered a standard myeloablative allogeneic hematopoietic stem cell transplant (alloHSCT).
Embodiment 392. The method of embodiment 391, wherein the human subject is free of primary graft failure or secondary graft failure for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 393. The method of any one of embodiments 256-392, wherein incidence, severity, timing, or any combination thereof of Grade I to Grade IV acute GVHD decreases after administration of the multi-component cellular therapy, relative to a corresponding human subject that has been administered a standard myeloablative allogeneic hematopoietic stem cell transplant (alloHSCT).
Embodiment 394. The method of embodiment 393, wherein the human subject is free of Grade I to Grade IV acute GVHD for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 395. The method of any one of embodiments 256-394, wherein incidence of steroid-refractory acute GVHD decreases after administration of the multi-component cellular therapy, relative to a corresponding human subject that has been administered a standard myeloablative allogeneic hematopoietic stem cell transplant (alloHSCT).
Embodiment 396. The method of embodiment 395, wherein the human subject is free of steroid-refractory acute GVHD for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 397. The method of any one of embodiments 256-396, wherein GVHD and relapse-free survival (GFRS) increases after administration of the multi-component cellular therapy, relative to a corresponding human subject that has been administered a standard myeloablative allogeneic hematopoietic stem cell transplant (alloHSCT).
Embodiment 398. The method of embodiment 397, wherein the human subject experiences GFRS for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 399. The method of any one of embodiments 256-398, wherein relapse-free survival increases after administration of the multi-component cellular therapy, relative to a corresponding human subject that has been administered a standard myeloablative allogeneic hematopoietic stem cell transplant (alloHSCT).
Embodiment 400. The method of embodiment 399, wherein the human subject experiences relapse-free survival for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 401. The method of any one of embodiments 256-400, wherein incidence, severity, timing, or any combination thereof of moderate to severe chronic GVHD decreases after administration of the multi-component cellular therapy, relative to a corresponding human subject that has been administered a standard myeloablative allogeneic hematopoietic stem cell transplant (alloHSCT).
Embodiment 402. The method of embodiment 401, wherein the human subject is free of severe chronic GVHD for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 403. The method of any one of embodiments 256-402, wherein incidence, severity, timing, or any combination thereof of non-relapse mortality decreases after administration of the multi-component cellular therapy, relative to a corresponding human subject that has been administered a standard myeloablative allogeneic hematopoietic stem cell transplant (alloHSCT).
Embodiment 404. The method of embodiment 403, wherein the human subject does not experience non-relapse mortality for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 405. The method of any one of embodiments 256-404, Grade 3 or higher infections decreases after administration of the multi-component cellular therapy, relative to a corresponding human subject that has been administered a standard myeloablative allogeneic hematopoietic stem cell transplant (alloHSCT).
Embodiment 406. The method of embodiment 405, wherein the human subject is free of Grade 3 or higher infections for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 407. The method of any one of embodiments 256-406, wherein incidence, timing, or both incidence and timing of neutrophil engraftment increases after administration of the multi-component cellular therapy, relative to a corresponding human subject that has been administered a standard myeloablative allogeneic hematopoietic stem cell transplant (alloHSCT).
Embodiment 408. The method of embodiment 407, wherein neutrophil engraftment occurs approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 22 days or more, approximately 22 days or more, approximately 23 days or more, approximately 24 days or more, approximately 25 days or more, approximately 26 days or more, approximately 27 days or more, approximately 28 days or more, approximately 29 days or more, approximately 30 days or more, approximately 33 days or more, approximately 33 days or more, approximately 33 days or more, approximately 34 days or more, approximately 35 days or more, approximately 36 days or more, approximately 37 days or more, approximately 38 days or more, approximately 39 days or more, approximately 40 days or more, approximately 44 days or more, approximately 44 days or more, approximately 44 days or more, approximately 44 days or more, approximately 45 days or more, approximately 46 days or more, approximately 47 days or more, approximately 48 days or more, approximately 49 days or more, approximately 50 days or more, approximately 55 days or more, approximately 55 days or more, approximately 55 days or more, approximately 55 days or more, approximately 55 days or more, approximately 56 days or more, approximately 57 days or more, approximately 58 days or more, approximately 59 days or more, approximately 60 days or more, approximately 66 days or more, approximately 66 days or more, approximately 66 days or more, approximately 66 days or more, approximately 66 days or more, approximately 66 days or more, approximately 67 days or more, approximately 68 days or more, approximately 69 days or more, approximately 70 days or more, approximately 77 days or more, approximately 77 days or more, approximately 77 days or more, approximately 77 days or more, approximately 77 days or more, approximately 77 days or more, approximately 77 days or more, approximately 78 days or more, approximately 79 days or more, approximately 80 days or more, approximately 88 days or more, approximately 88 days or more, approximately 88 days or more, approximately 88 days or more, approximately 88 days or more, approximately 88 days or more, approximately 88 days or more, approximately 88 days or more, approximately 89 days or more, or approximately 90 days or more after administration of the multi-component cellular therapy.
Embodiment 409. The method of any one of embodiments 256-408, wherein incidence, timing, or both incidence and timing of platelet engraftment increases after administration of the multi-component cellular therapy, relative to a corresponding human subject that has been administered a standard myeloablative allogeneic hematopoietic stem cell transplant (alloHSCT).
Embodiment 410. The method of embodiment 409, wherein platelet engraftment occurs approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 22 days or more, approximately 22 days or more, approximately 23 days or more, approximately 24 days or more, approximately 25 days or more, approximately 26 days or more, approximately 27 days or more, approximately 28 days or more, approximately 29 days or more, approximately 30 days or more, approximately 33 days or more, approximately 33 days or more, approximately 33 days or more, approximately 34 days or more, approximately 35 days or more, approximately 36 days or more, approximately 37 days or more, approximately 38 days or more, approximately 39 days or more, approximately 40 days or more, approximately 44 days or more, approximately 44 days or more, approximately 44 days or more, approximately 44 days or more, approximately 45 days or more, approximately 46 days or more, approximately 47 days or more, approximately 48 days or more, approximately 49 days or more, approximately 50 days or more, approximately 55 days or more, approximately 55 days or more, approximately 55 days or more, approximately 55 days or more, approximately 55 days or more, approximately 56 days or more, approximately 57 days or more, approximately 58 days or more, approximately 59 days or more, approximately 60 days or more, approximately 66 days or more, approximately 66 days or more, approximately 66 days or more, approximately 66 days or more, approximately 66 days or more, approximately 66 days or more, approximately 67 days or more, approximately 68 days or more, approximately 69 days or more, approximately 70 days or more, approximately 77 days or more, approximately 77 days or more, approximately 77 days or more, approximately 77 days or more, approximately 77 days or more, approximately 77 days or more, approximately 77 days or more, approximately 78 days or more, approximately 79 days or more, approximately 80 days or more, approximately 88 days or more, approximately 88 days or more, approximately 88 days or more, approximately 88 days or more, approximately 88 days or more, approximately 88 days or more, approximately 88 days or more, approximately 88 days or more, approximately 89 days or more, or approximately 90 days or more after administration of the multi-component cellular therapy.
Embodiment 411. The method of any one of embodiments 256-410, wherein overall survival increases after administration of the multi-component cellular therapy, relative to a corresponding human subject that has been administered a standard myeloablative allogeneic hematopoietic stem cell transplant (alloHSCT).
Embodiment 412. The method of embodiment 411, wherein the human subject experiences overall survival for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 413. The method of any one of embodiments 256-412, wherein incidence of re-hospitalization decreases after administration of the multi-component cellular therapy, relative to a corresponding human subject that has been administered a standard myeloablative allogeneic hematopoietic stem cell transplant (alloHSCT).
Embodiment 414. The method of embodiment 413, wherein the human subject does not require re-hospitalization for approximately 1 month or more, approximately 2 months or more, approximately 3 months or more, approximately 6 months or more, approximately 9 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the multi-component cellular therapy.
Embodiment 415. A cellular therapy kit comprising the multi-component cellular therapy product of any one of embodiments 1-51.
Embodiment 416. A cellular therapy kit comprising the multi-component pharmaceutical treatment of any one of embodiments 109-138 or 181-193.
Embodiment 417. A cellular therapy kit comprising:
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, wherein the first population of CD45⁺ cells is formulated with an excipient at a neutral pH;
b) a second single dose transfer bag comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, wherein the second population of isolated CD45⁺ cells is formulated with an excipient at a neutral pH; and
c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons wherein the third population of isolated CD45⁺ cells is formulated with an excipient at a neutral pH, wherein the excipient comprises one more cryoprotectants; or
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, wherein the first population of isolated CD45⁺ cells and the second population of isolated CD45⁺ cells are formulated with an excipient at a neutral pH; and
b) a second single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, wherein the third population of isolated CD45⁺ cells is formulated with an excipient at a neutral pH, wherein the excipient comprises one more cryoprotectants.
Embodiment 418. A cellular therapy kit comprising:
a) a container comprising a unit dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and one or more excipients;
b) a container comprising a unit dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and ore or more excipients; and
c) a container comprising a unit dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients; or
a) a container comprising a unit dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, a unit dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and one or more excipients; and
b) a container comprising a unit dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients.
Embodiment 419. The cellular therapy kit of any one of embodiments 415-418, wherein the kit further comprises written instructions for using the cellular therapy for treating a hematologic malignancy in a human subject.
Embodiment 420. The cellular therapy kit of any one of embodiments 415-418, wherein the kit further comprises written instructions for using the cellular therapy for treating multiple sclerosis in a human subject.
Embodiment 421. A unit dose comprising the multi-component cellular therapy product of any one of embodiments 1-51.
Embodiment 422. A unit dose comprising the multi-component pharmaceutical treatment of any one of embodiments 109-138 or 181-193.
Embodiment 423. A unit dose comprising:
a) a first population of isolated CD45⁺ cells comprising approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs;
b) a second population of isolated CD45⁺ cells comprising approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs; and/or
c) a third population of isolated CD45⁺ cells comprising approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons.
Embodiment 424. The unit dose of embodiment 423, wherein each population of isolated CD45⁺ cells further comprises one or more excipients.
Embodiment 425. The unit dose of any one of embodiments 423 or embodiment 424, wherein the first population of isolated CD45⁺ cells, the second population of isolated CD45⁺ cells, and the third population of isolated CD45⁺ cells are each provided as separate unit doses.
Embodiment 426. The unit dose of embodiment 423 or embodiment 424, wherein the first population of isolated CD45⁺ cells and the second population of isolated CD45⁺ cells are provided as a single unit dose.
Embodiment 427. The unit dose of embodiment 423 or embodiment 424, wherein the first population of isolated CD45⁺ cells and the third population of isolated CD45⁺ cells are provided as a single unit dose.
Embodiment 428. The unit dose of embodiment 423 or embodiment 424, wherein the second population of isolated CD45⁺ cells and the third population of isolated CD45⁺ cells are provided as a single unit dose.
Embodiment 429. The unit dose of embodiment 423 or embodiment 424, wherein the first population of isolated CD45⁺ cells, the second population of isolated CD45⁺ cells, and the third population of isolated CD45⁺ cells are provided as a single unit dose.
Embodiment 430. A unit dose comprising:
a) approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and one or more excipients;
b) approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and ore or more excipients; and
c) approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients.
Embodiment 431. The unit dose of any one of embodiments 430, wherein the HSPCs, the fresh Tregs, and/or the Tcons are provided as separate unit doses.
Embodiment 432. The unit dose of any one of embodiments 430, wherein the HSPCs and the fresh Tregs are provided as a single unit dose.
Embodiment 433. The unit dose of any one of embodiments 430, wherein the HSPCs and the Tcons are provided as a single unit dose.
Embodiment 434. The unit dose of any one of embodiments 430, wherein the fresh Tregs and the Tcons are provided as a single unit dose.
Embodiment 435. The unit dose of any one of embodiments 430, wherein the HSPCs, the fresh Tregs, and the Tcons are provided as a single unit dose.
Embodiment 436. An article of manufacture comprising the multi-component cellular therapy product of any one of embodiments 1-51.
Embodiment 437. An article of manufacture comprising the multi-component pharmaceutical treatment of any one of embodiments 109-138 or 181-193.
Embodiment 438. An article of manufacture comprising:
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, wherein the first population of CD45⁺ cells is formulated with an excipient at a neutral pH;
b) a second single dose transfer bag comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, wherein the second population of isolated CD45⁺ cells is formulated with an excipient at a neutral pH; and
c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons wherein the third population of isolated CD45⁺ cells is formulated with an excipient at a neutral pH, wherein the excipient comprises one more cryoprotectants; or
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, wherein the first population of CD45⁺ cells and the second population of CD45⁺ cells are formulated with an excipient at a neutral pH; and
b) a second single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, wherein the third population of isolated CD45⁺ cells is formulated with an excipient at a neutral pH, wherein the excipient comprises one more cryoprotectants.
Embodiment 439. An article of manufacture comprising:
a) a container comprising a unit dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and one or more excipients;
b) a container comprising a unit dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and ore or more excipients; and
c) a container comprising a unit dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients; or
a) a container comprising a unit dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, a unit dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and one or more excipients; and
b) a container comprising a unit dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients.
Embodiment 440. The article of manufacture of any one of embodiments 436-439, further comprising instructions for administering the article of manufacture to a human subject treat a hematologic malignancy.
Embodiment 441. The article of manufacture of any one of embodiments 436-439, further comprising instructions for administering the article of manufacture to a human subject treat multiple sclerosis.
Embodiment 442. A container comprising the multi-component cellular therapy product of any one of embodiments 1-51.
Embodiment 443. A container comprising the multi-component pharmaceutical treatment of any one of embodiments 109-138 or 181-193.
Embodiment 444. A container comprising:
a) a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, wherein the first population of CD45⁺ cells is formulated with an excipient at a neutral pH;
b) a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, wherein the second population of isolated CD45⁺ cells is formulated with an excipient at a neutral pH; and
c) a third population of isolated CD45⁺ cells comprising a dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, wherein the third population of isolated CD45⁺ cells is formulated with an excipient at a neutral pH, wherein the excipient comprises one more cryoprotectants; or
a) a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, wherein the first population of CD45⁺ cells and the population of CD45⁺ cells are formulated with an excipient at a neutral pH; and
b) a third population of isolated CD45⁺ cells comprising a dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, wherein the third population of isolated CD45⁺ cells is formulated with an excipient at a neutral pH, wherein the excipient comprises one more cryoprotectants.
Embodiment 445. The container of embodiment 444, wherein the first population of isolated CD45⁺ cells, the second population of isolated CD45⁺ cells, and/or the third population of isolated CD45⁺ cells are comprised in a separate container.
Embodiment 446. The container of embodiment 444, wherein the first population of isolated CD45⁺ cells and the second population of isolated CD45⁺ cells are comprised in a single container.
Embodiment 447. The container of embodiment 444, wherein the first population of isolated CD45⁺ cells and third population of isolated CD45⁺ cells are comprised in a single container.
Embodiment 448. The container of embodiment 444, wherein the second population of isolated CD45⁺ cells and third population of isolated CD45⁺ cells are comprised in a single container.
Embodiment 449. The container of embodiment 444, wherein the first population of isolated CD45⁺ cells, the second population of isolated CD45⁺ cells, and the third population of isolated CD45⁺ cells are comprised in a single container.
Embodiment 450. A container comprising:
a) a unit dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, and one or more excipients;
b) a unit dose of approximately 1.0×10⁵ to approximately 2.0×10⁷ fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and ore or more excipients; and
c) a unit dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients; or
a) a unit dose of approximately 1.0×10⁵ to approximately 1.0×10⁸ CD34⁺ hematopoietic stem and progenitor cells (HSPCs) per kilogram of body weight of a human subject receiving the therapy, approximately 5.0×10⁵ to approximately 5.0×10⁸ HSPCs, or approximately 1.5×10⁷ to approximately 1.5×10¹⁰ HSPCs, a unit dose of approximately 1.0×10⁵ to approximately 2.0×107 fresh CD4⁺CD25⁺CD127^{dim} regulatory T cells (Tregs) per kilogram of body weight of the human subject receiving the therapy, approximately 1.5×10⁷ to approximately 3.0×10⁹ fresh Tregs, or approximately 5.0×10⁵ to approximately 1.0×10⁸ fresh Tregs, and one or more excipients; and
b) a unit dose of approximately 1.0×10⁵ to approximately 4.0×10⁷ conventional CD3⁺ T cells (Tcons) per kilogram of body weight of the human subject receiving the therapy, approximately 5.0×10⁵ to approximately 2.0×10⁸ Tcons, or approximately 1.5×10⁷ to approximately 6.0×10⁹ Tcons, and one or more excipients.
Embodiment 451. The container of embodiment 450, wherein the HSPCs, the fresh Tregs, and/or the Tcons are comprised in a separate container.
Embodiment 452. The container of embodiment 450, wherein the HSPCs and the fresh Tregs are comprised in a single container.
Embodiment 453. The container of embodiment 450, wherein the HSPCs and the Tcons are comprised in a single container.
Embodiment 454. The container of embodiment 450, wherein the fresh Tregs and the Tcons are comprised in a single container.
Embodiment 455. The container of embodiment 450, wherein the HSPCs, the fresh Tregs, and the Tcons are comprised in a single container.

### EXAMPLES

The following examples are included for illustrative purposes only and are not intended to limit the scope of the disclosure.

### Example 1: Clinical Study

### A. Study Design

A clinical study was conducted in subjects with advanced hematologic malignancies undergoing myeloablative allogeneic hematopoietic cell transplantation (alloHSCT).

The graft composition and manufacturing processes are detailed below.

Primary endpoints of the study include the incidence of primary graft failure; and the incidence, severity, and timing of Grade III-V acute GVHD.

Secondary endpoints for all groups include neutrophil engraftment, platelet engraftment, incidence of secondary graft failure, incidence and severity of treatment-emergent adverse events (TEAEs), incidence and severity of steroid-refractory acute GVHD, for example, grade 3-4 steroid-refractory acute GVHD, incidence and severity of chronic GVHD, incidence of post-transplant lymphoproliferative disorder (PTLD), non-relapse mortality (NRM), disease relapse (Arms I & III), relapse free survival, GVHD and relapse free survival (GRFS), overall survival, incidence of serious infections, and T cell immunity reconstitution parameters.

### B. Study Populations

Subjects were eligible to receive a composition of the disclosure if they met all the following criteria:
(1) Age ≥18 and ≤65 years at the time of enrollment.
(2) Diagnosed with the any of the following histopathologically-confirmed diseases: (a) Acute myeloid, lymphoid or mixed phenotype leukemia in complete remission (CR) or CR with incomplete hematologic recovery (CRi) without the presence of known minimal residual disease; or (b) Acute myeloid, lymphoid or mixed phenotype leukemia that is either: (i) not in morphologic CR with bone marrow infiltration by leukemic blasts of ≤10%, or (ii) in morphologic CR with evidence of minimal residual positivity by either multiparameter flow cytometric analysis or by a nucleic acid-based technique; (c) High or Very High-risk Myelodysplastic syndromes; (d) Myelofibrosis (MF) that is eligible for transplant per National Comprehensive Cancer Network Guidelines. Specifically, patients should be diagnosed with MF that is either: (i) intermediate-2- or high-risk according to the IPSS, DIPSS or DIPSS-plus scoring systems; or (ii) intermediate-1-risk disease associated with high-risk features such as high symptoms burden, low platelet counts, or complex cytogenetics; per NCCN guidelines and Investigator judgement (e) myeloproliferative syndromes; (f) Non-Hodgkin lymphoma with poor risk features not suitable for autologous HCT.
(3) Planning to undergo myeloablative allogeneic hematopoietic cell transplantation (MA-alloHSCT) including a suitable myeloablative conditioning regimen.
(4) Matched to a donor that is a: (a) Matched sibling donor who is an 8/8 match for HLA-A, -B, -C, and -DRB1, all typed using DNA-based high-resolution methods; or (b) Matched unrelated donor who is an 8/8 match at HLA-A, -B, -C, and -DRB1, all typed using DNA-based high resolution methods.
(5) Estimated glomerular filtration rate (eGFR)>30 mL/minute; or >50 mL/minute for patients for whom GVHD prophylaxis with tacrolimus is planned.
(6) Cardiac ejection fraction at rest ≥45% or shortening fraction of ≥27% by echocardiogram or radionuclide scan (MUGA).
(7) Diffusing capacity of the lung for carbon monoxide (DLCO) (adjusted for hemoglobin) ≥50%.
(8) Negative serum or urine beta-HCG test in females of childbearing potential within 3 weeks of registration.
(9) Total bilirubin <2 times upper limit of normal (ULN) (patients with Gilbert's syndrome may be included where hemolysis has been excluded and with approval of the medical monitor).
(10) ALT/AST <3 times upper limit of normal (ULN).

Subjects were ineligible to receive the composition of the disclosure if they met any of the following exclusion criteria:
(1) Received a prior allogeneic HCT.
(2) Candidate for autologous transplant.
(3) Currently receiving corticosteroids or other immunosuppressive therapy. Topical corticosteroids or oral systemic corticosteroid doses less than or equal to 10 mg/day are allowed.
(4) Planned donor lymphocyte infusion (DLI) recipient.
(5) Planned recipient of pharmaceutical in vivo or ex vivo T cell depletion, e.g., post-transplant cyclophosphamide (Cy), peri-transplant anti-thymocyte globulin (ATG), or alemtuzumab. For patients that have previously been exposed to a T cell-depleting agent, a 5-half-life washout of the agent must occur prior to planned Day 0 (day of infusion of the Treg and HSPC components of the graft).
(6) Positive for anti-donor HLA antibodies against a mismatched allele in the selected donor as determined by either: (a) A positive crossmatch test of any titer; or (b) The presence of anti-donor HLA antibody to any HLA locus.
(7) Karnofsky performance score <70%.
(8) Hematopoietic cell transplantation-specific Comorbidity Index (HCT-CI)>4.
(9) Uncontrolled bacterial, viral, or fungal infections (currently taking antimicrobial therapy and with progression or no clinical improvement) at time of enrollment.
(10) Seropositive for HIV-1 or -2, HTLV-1 or -2, Hepatitis B sAg, or Hepatitis C antibody,
(11) Known allergy or hypersensitivity to, or intolerance of, tacrolimus (or tacrolimus and sirolimus if eligible for single-agent prophylaxis with either)
(12) Documented allergy or hypersensitivity to iron dextran or bovine, murine, algal or Streptomyces avidinii proteins.
(13) Any uncontrolled autoimmune disease requiring active immunosuppressive treatment.
(14) Concurrent malignancies or active disease within 1 year, except non-melanoma skin cancers that have been curatively resected.
(15) Psychosocial circumstances that preclude the patient being able to go through transplant or participate responsibly in follow up care.
(16) Women who are pregnant or breastfeeding.
(17) Women of childbearing potential (WOCBP) or men who have sexual contact with WOCBP unwilling to use effective forms of birth control or abstinence for one year after transplantation.
(18) Any serious medical condition or abnormality in clinical laboratory tests that, in the investigator's or Medical Monitor's judgment, precludes the recipient's safe participation in and completion of the study, or which could affect compliance with the protocol or interpretation of results.

Subjects were part of the following groups:
Arm 1: subjects planning to undergo myeloablative allogeneic hematopoietic cell transplantation (MA-alloHSCT) for the treatment of either acute myeloid, lymphoid, or mixed phenotype leukemia in complete remission (CR) or CR with incomplete hematologic recovery (CRi) with no known minimal residual disease positivity, planning to MA-alloHSCT.
Arm 2: subjects planning to undergo MA-alloHSCT for acute myeloid, lymphoid, or mixed phenotype leukemia that is either: (i) not in morphologic CR with bone marrow infiltration by leukemic blasts of ≤10%, or (ii) in morphologic CR with evidence of minimal residual positivity by either multiparameter flow cytometric analysis or by a nucleic acid-based technique.
Arm 3: subjects planning to undergo MA-alloHSCT for high or very high-risk myelodysplastic syndrome (MDS) myelodysplastic syndromes or for myelofibrosis (primary myelofibrosis or myelofibrosis evolved from other myeloproliferative neoplasms).

Subjects with sensitivity to iron dextran, chemical products derived from cyanine dyes, and proteins products derived from murine, bovine, algal, and Streptomyces avidinii sources are excluded.

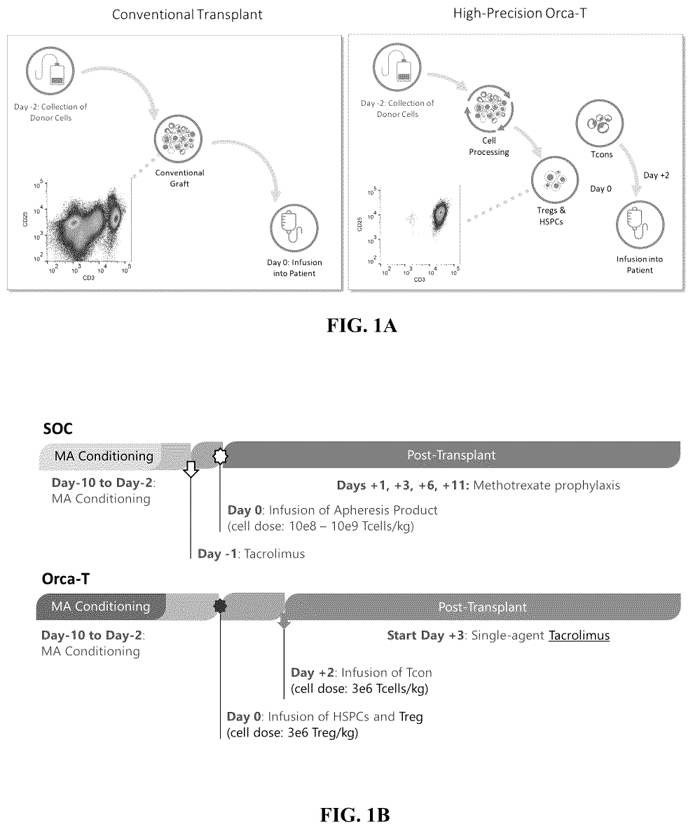

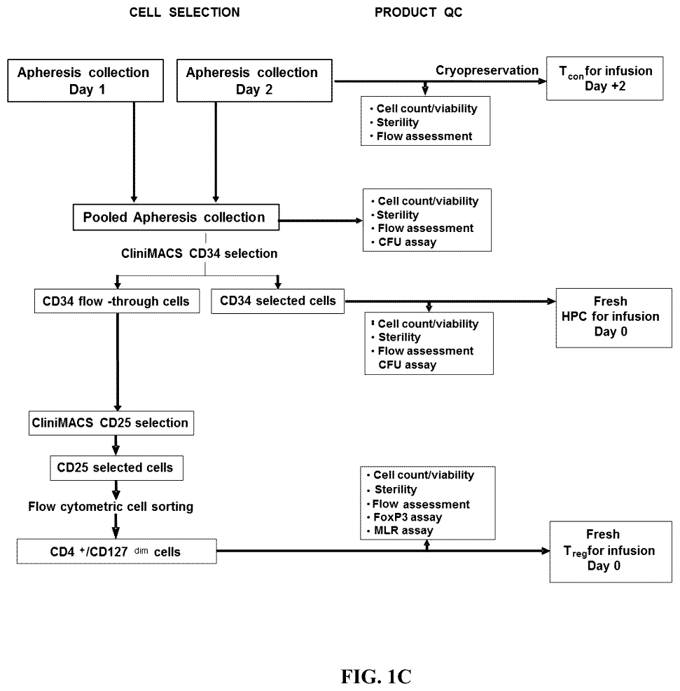

FIGS. 1A-B illustrate the schematics of the transplant according to the present study (identified as High-Precision Orca-T or OrcaT) and the differences compared to a standard of care (SOC) cohort (identified as Conventional Transplant or SOC). FIG. 1C illustrates a schematic of graft production and administration.

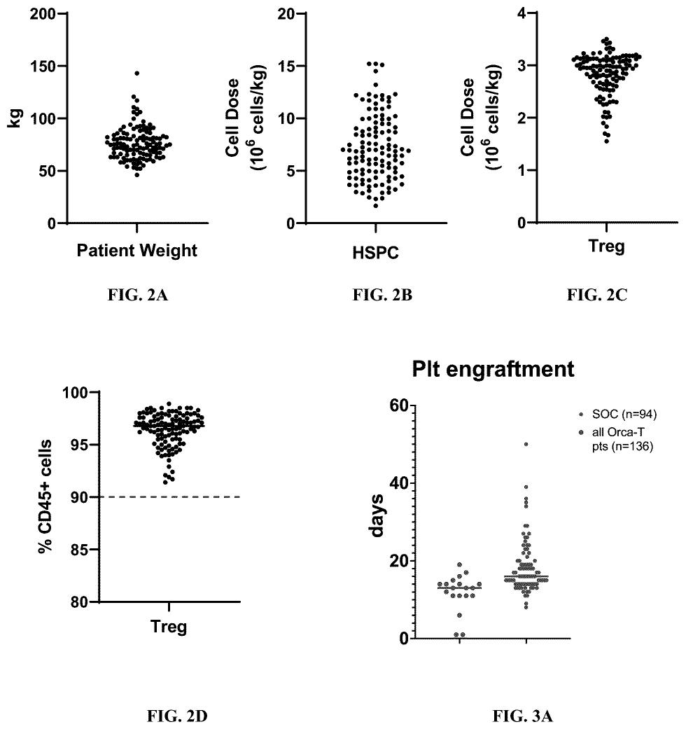

FIG. 2A illustrates the weights of patients enrolled in the study. Table 7 shows the shows the demographics, primary disease, features determining disease risk status, disease status at transplant, and transplant details for a representative subset of the subjects. Abbreviations: DLBCL, diffuse large B cell lymphoma; AML, acute myeloid leukemia; ALL, acute lymphocytic leukemia; MDS, myelodysplastic syndrome; MF, myelofibrosis; MPAL, mixed phenotype acute leukemia; CML, chronic myeloid leukemia; CR1, 1st complete remission; CR2, 2nd complete remission; active, active disease (not in CR as defined for disease entity); MRD⁺, minimal residual disease positive; MRD, matched related donor; URD, unrelated donor; MF, myelofibrosis. FTBI, fractionated total body irradiation; Cy, cyclophosphamide; VP-16, etoposide; Bu, busulfan; FLT3⁺, FMS-like tyrosine kinase 3; c-kit, ASXL1, additional sex combs like 1. Days of follow up denotes the number of days since transplant Day 0 for each patient at the time of reporting.

TABLE 7

| | | | | | | | | Days |
|---|---|---|---|---|---|---|---|---|
| | | | | Disease | | | | of |
| | | | High-risk | status at | Donor | Conditioning | GVHD | follow |
| Age | Sex | Disease | Features | transplant | type | Regimen | prophylaxis | up |
| 49 | M | BLBCL | refractory | active | MRD | BCNU/VP16/Cy | Sirolimus | 1219 |
| 42 | M | AML | FLT3⁺ | CR2 | MRD | BU/Cy | Sirolimus to | 1177 |
| | | | | | | | Tacrolimus | |
| 34 | M | ALL | refractory | active | MRD | TBI/Cy/VP16 | Sirolimus | 1107 |
| 20 | M | ALL | MRD⁺ | CR2 | MRD | BU/Cy | Sirolimus | 1079 |
| 54 | M | CML | refractory | active | MRD | BU/Cy | Sirolimus | 1058 |
| 53 | M | AML | AML CR2 | CR2 | MRD | BU/Cy | Sirolimus | 981 |
| 56 | M | MF | refractory | active | MRD | BU/Cy | Tacrolimus | 932 |
| 40 | F | AML | refractory | CR1 | MRD | BU/Cy | Tacrolimus | 260 |
| 59 | M | PTCL | refractory | active | MRD | BCNU/VP16/Cy | Sirolimus | 225 |
| 42 | F | AML | refractory | CR1 | MRD | BU/Cy | None | 211 |
| 40 | M | AML | refractory | CR1 | MRD | BU/Cy | None | 204 |
| 48 | M | MDS | Monosomy | active | MRD | BU/Cy | None | 190 |
| | | | 7 | | | | | |
| 35 | M | ALL | MRD⁺ | CR1 | MRD | FTBI/VP16/Cy | Sirolimus | 176 |
| 28 | M | ALL | CR2 | CR2 | MRD | BU/Cy | None | 127 |
| 56 | F | MF | n/a | active | URD | BU/Cy | Tacrolimus | 119 |
| 39 | M | MPAL | MPAL | CR1 | MRD | FTBI/VP16/Cy | Sirolimus | 120 |
| 41 | F | AML | TP53 and c- | CR1 | MRD | BU/Cy | Tacrolimus | 99 |
| | | | Kit mutated | | | | | |
| 39 | M | ALL | Ph-like | CR1 | MRD | FTBI/VP16/Cy | None | 43 |
| 34 | M | MPAL | Complex | CR1 | MRD | FTBI/VP16/Cy | Sirolimus | 36 |
| | | | karyotype, | | | | | |
| | | | MPAL | | | | | |
| 26 | F | AML | AML t(6:9) | CR1 | MRD | BU/Cy | Tacrolimus | 34 |
| 52 | M | AML | ASXL1 | CR1 | MRD | BU/Cy | Tacrolimus | 22 |
| | | | mutation | | | | | |

### Standard of Care Control Cohort

For comparison of clinical outcomes, a standard of care (SOC) comparator cohort was identified retrospectively from subjects who received a SOC myeloablative alloHSCT at one of the same clinical sites during the same period; a schematic of the protocol followed for SOC patients is also shown in FIGS. 1A-B. To be included in the SOC cohort, subjects had to meet all the following criteria: (a) they were diagnosed with a hematologic malignancy eligible for treatment with an alloHSCT; (b) they received an allograft of mobilized peripheral blood (i.e., not a bone marrow-derived graft); (c) their donor was a fully HLA-matched related donor (unrelated donors were not included due to the limited number in the treatment cohort to date); (d) they received a myeloablative conditioning regimen; and (e) they were not enrolled on an investigative protocol. Personnel who identified subjects for the SOC cohort were blinded as to their clinical outcomes.

All patients in the SOC cohort received myeloablative conditioning regimens and dual-agent GVHD prophylaxis with tacrolimus and methotrexate.

Clinical characteristics of subjects in the SOC cohort are provided in TABLE 8.

TABLE 8: characteristics of a representative subset of subjects in the standard of care (SOC) cohort. Abbreviations used: DLBCL, diffuse large B cell lymphoma; AML, acute myeloid leukemia; ALL, acute myeloid leukemia; MDS, myelodysplastic syndrome; MF, myelofibrosis; MPAL, mixed phenotype acute leukemia; CML, chronic myeloid leukemia; CR1, 1st complete remission; CR2, 2nd complete remission; “active”, active disease (not CR as defined for each disease entity); MRD, minimal residual disease; MRD, matched related donor; MF, myelofibrosis. FTBI, fractionated total body irradiation; Cy, cyclophosphamide; VP-16, etoposide; Bu, busulfan; FLT3⁺, FMS-like tyrosine kinase 3; Tac, tacrolimus; MTX, methotrexate.

TABLE 8

| | | | Disease | | | |
|---|---|---|---|---|---|---|
| | | | Status at | Donor | Conditioning | GVHD |
| Age | Sex | Disease | Transplant | Type | regimen | Prophylaxis |
| 46 | F | ALL | CR 1 | MRD | FTBI/VP16 | FK/MTX |
| 49 | F | ALL | CR 1 | MRD | FTBI/VP16 | FK/MTX |
| 56 | F | AML | CR 1 | MRD | BU/CY | FK/MTX |
| 58 | M | AML | CR 1 | MRD | BU/CY | FK/MTX |
| 44 | M | ALL | CR 1 | MRD | FTBI/VP16 | FK/MTX |
| 45 | M | AML | CR 2 | MRD | FTBI/VP16 | FK/MTX |
| 52 | M | ALL | CR 1 | MRD | BU/CY | FK/MTX |
| 56 | M | AML | CR 1 | MRD | BU/CY | FK/MTX |
| 32 | F | ALL | CR 1 | MRD | FTBI/VP16 | FK/MTX |
| 23 | M | AML | CR 2 | MRD | BU/CY | FK/MTX |
| 59 | F | ALL | CR 1 | MRD | BU/CY | FK/MTX |
| 50 | F | AML | CR 2 | MRD | BU/CY | FK/MTX |
| 50 | M | ALL | CR 1 | MRD | FTBI/VP16 | FK/MTX |
| 31 | F | AML | CR 1 | MRD | BU/CY | FK/MTX |
| 57 | F | AML | CR 1 | MRD | BU/CY | FK/MTX |
| 21 | M | AML | CR 1 | MRD | BU/CY | FK/MTX |
| 27 | M | OAL | CR 2 | MRD | BU/CY | FK/MTX |
| 51 | F | ALL | CR 1 | MRD | FTBI/VP16 | FK/MTX |
| 38 | M | ALL | CR 1 | MRD | FTBI/VP16 | FK/MTX |
| 44 | F | CML | CP1 | MRD | BU/CY | FK/MTX |
| 19 | M | ALL | CR 1 | MRD | FTBI/VP16 | FK/MTX |
| 58 | M | ALL | CR 1 | MRD | BU/CY | FK/MTX |
| 55 | M | ALL | CR 1 | MRD | BU/CY | FK/MTX |
| 39 | M | AML | active | MRD | BU/CY | FK/MTX |
| 25 | M | AML | CR 2 | MRD | BU/CY | FK/MTX |
| 33 | M | NHL | CR 1 | MRD | BU/CY | FK/MTX |
| 32 | M | AML | CR 2 | MRD | BU/CY | FK/MTX |
| 59 | M | AML | CR 1 | MRD | BU/CY | FK/MTX |
| 47 | M | AML | CR 1 | MRD | BU/CY | FK/MTX |
| 47 | M | ALL | CR 1 | MRD | FTBI/VP16 | FK/MTX |
| 43 | M | AML | CR 1 | MRD | BU/CY | FK/MTX |
| 59 | F | ALL | CR 2 | MRD | BU/CY | FK/MTX |
| 27 | M | ALL | CR 1 | MRD | FTBI/VP16 | FK/MTX |
| 40 | M | AML | CR 1 | MRD | BU/CY | FK/MTX |
| 24 | F | ALL | CR 1 | MRD | FTBI/VP16 | FK/MTX |
| 27 | F | NHL | active | MRD | BU/CY | FK/MTX |
| 34 | M | ALL | CR 1 | MRD | FTBI/VP16 | FK/MTX |
| 58 | M | AML | CR 1 | MRD | BU/CY | FK/MTX |
| 41 | M | CML | active | MRD | BU/CY | FK/MTX |
| 58 | M | ALL | CR 1 | MRD | BU/CY | FK/MTX |
| 52 | F | AML | active | MRD | BU/CY | FK/MTX |
| 62 | M | MDS | active | MRD | BU/CY | FK/MTX |
| 62 | M | MDS | active | MRD | BU/CY | FK/MTX |
| 40 | M | AML | CR 1 | MRD | BU/CY | FK/MTX |
| 44 | F | AML | CR 1 | MRD | FTBI/VP16 | FK/MTX |
| 54 | M | ALL | CR 1 | MRD | BU/CY | FK/MTX |
| 36 | M | AML | CR 1 | MRD | BU/CY | FK/MTX |
| 46 | F | CML | CR 2 | MRD | FTBI/VP16 | FK/MTX |
| 41 | M | MDS | active | MRD | BU/CY | FK/MTX |
| 43 | F | ALL | CR 2 | MRD | FTBI/VP16 | FK/MTX |
| 51 | F | MDS | active | MRD | BU/CY | FK/MTX |
| 54 | M | MDS | active | MRD | BU/CY | FK/MTX |
| 52 | F | CML | CR1 | MRD | BU/CY | FK/MTX |
| 56 | F | MDS | active | MRD | BU/CY | FK/MTX |
| 60 | M | AML | CR 1 | MRD | BU/CY | FK/MTX |
| 56 | M | MDS | active | MRD | BU/CY | FK/MTX |
| 57 | F | MDS | active | MRD | BU/CY | FK/MTX |
| 49 | F | ALL | CR 1 | MRD | FTBI/VP16 | FK/MTX |
| 34 | M | AML | CR 1 | MRD | BU/CY | FK/MTX |
| 59 | F | ALL | CR 1 | MRD | BU/CY | FK/MTX |
| 60 | M | MDS | active | MRD | BU/CY | FK/MTX |
| 32 | M | AML | CR 1 | MRD | FTBI/VP16 | FK/MTX |
| 57 | F | OAL | CR | MRD | BU/CY | FK/MTX |
| 43 | F | AML | CR 1 | MRD | BU/CY | FK/MTX |
| 47 | M | ALL | CR 1 | MRD | BU/CY | FK/MTX |
| 60 | M | MDS | active | MRD | BU/CY | FK/MTX |
| 63 | M | AML | CR 1 | MRD | BU/CY | FK/MTX |
| 49 | M | AML | CR 1 | MRD | BU/CY | FK/MTX |
| 51 | F | AML | CR 2 | MRD | BU/CY | FK/MTX |
| 49 | M | AML | CR 2 | MRD | BU/CY | FK/MTX |
| 40 | F | ALL | CR 1 | MRD | BU/CY | FK/MTX |
| 48 | M | NHL | CR 2 | MRD | BU/CY | FK/MTX |
| 52 | M | AML | CR 2 | MRD | BU/CY | FK/MTX |
| 36 | M | OAL | CR 1 | MRD | FTBI/VP16 | FK/MTX |
| 27 | M | NHL | active | MRD | FTBI/VP16 | FK/MTX |
| 57 | M | AML | CR 1 | MRD | BU/CY | FK/MTX |
| 56 | F | NHL | active | MRD | BU/CY | FK/MTX |
| 52 | F | AML | CR 1 | MRD | BU/CY | FK/MTX |
| 39 | M | AML | active | MRD | FTBI/VP16 | FK/MTX |
| 57 | F | MDS | active | MRD | BU/CY | FK/MTX |
| 26 | M | ALL | CR 1 | MRD | FTBI/VP16 | FK/MTX |
| 21 | F | ALL | CR3⁺ | MRD | FTBI/VP16 | FK/MTX |
| 55 | M | NHL | active | MRD | BU/CY | FK/MTX |
| 25 | M | ALL | CR 1 | MRD | FTBI/VP16 | FK/MTX |
| 34 | F | MDS | active | MRD | BU/CY | FK/MTX |
| 40 | F | AML | CR 1 | MRD | BU/CY | FK/MTX |
| 59 | M | ALL | CR 1 | MRD | BU/CY | FK/MTX |
| 42 | F | NHL | active | MRD | BU/CY | FK/MTX |
| 29 | M | ALL | CR 1 | MRD | FTBI/VP16 | FK/MTX |
| 26 | F | ALL | CR | MRD | FTBI/VP16 | FK/MTX |
| 40 | M | CML | CP1 | MRD | BU/CY | FK/MTX |
| 58 | F | MDS | CR | MRD | BU/CY | FK/MTX |
| 53 | M | ALL | CR 1 | MRD | BU/CY | FK/MTX |
| 21 | M | ALL | CR 2 | MRD | BU/CY | FK/MTX |

### C. Donors

HLA-identical related or unrelated donors were used.

Donors were used that met all of the following inclusion criteria:
(1) Age ≥16 and ≤75 years at time of enrollment
(2) Matched to the patient as follows: Either one of: (i) matched related donor who is an 8/8 match for HLA-A, -B, -C, and -DRB1, all typed using DNA-based high resolution methods; (ii) matched unrelated donor who is an 8/8 match at HLA-A, -B, -C, and -DRB1, all typed using DNA-based high resolution methods.
(3) Able to donate at a site that will employ a Spectra Optia Apheresis System for post-mobilization apheresis.
(4) Meets federal eligibility criteria for donors of viable, leukocyte-rich cells or tissues as defined by 21 CFR § 1271 2018 and all relevant FDA Guidance for Industry (Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; Donor Screening Recommendations to Reduce the Risk of Transmission of Zika Virus by Human Cells, Tissues, and Cellular and Tissue-Based Products, 2018).
(5) Meets any other criteria for donation as specified by standard NMDP guidelines (NMDP donors) or institutional standards (non-NMDP donors).
(6) Female donors of child-bearing potential must have a negative serum or urine beta HCG test within 3 weeks of mobilization.
(7) Capable of undergoing leukapheresis, have adequate venous access, and be willing to undergo insertion of a central catheter should leukapheresis via peripheral vein be inadequate.

Donors determined to be ineligible, based on the results of required testing and/or screening, were nonetheless included if either apply, as per 21 CFR § 1271.65 2018: (a) the donor is a first-degree or second-degree blood relative of the recipient; or (b) Urgent medical need, meaning no comparable human cell product was available and the recipient is likely to suffer death or serious morbidity without the human cell product, as attested by the Investigator.

Donors meeting any of the following exclusion criteria were not eligible:
(1) Evidence of active infection
(2) Seropositive for HIV-1 or -2, HTLV-1 or -2
(3) Positive for anti-hepatitis C (HCV) antibody or HCV NAT.
(4) Positive serologic or PCR test results indicating acute or chronic HBV infection. Donors whose HBV infection status cannot be determined conclusively by serologic test results must be negative for HBV by PCR to be eligible for study participation.
(5) Potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). Donors determined to be ineligible based on the results of Zika virus screening may be determined to be eligible if: (a) the donor has no signs or symptoms consistent with active Zika virus infection; and (b) The donor is a first-degree or second-degree blood relative of the recipient, or ii) in cases of urgent medical need, meaning no comparable human cell product is available and the recipient is likely to suffer death or serious morbidity without the human cell product, as attested by the Investigator.
(6) Women who are pregnant or breastfeeding.

### D. Generation of Cell Components

Donors received mobilization therapy with daily G-CSF. The recommended dose was 10 μg/kg/day SQ (rounded off to the nearest vial size of either 300 or 480 μg). The Mobilization Phase started on the first day of administration of G-CSF and continued until the final day of leukapheresis. A schematic of graft production and administration for the protocol is provided in FIG. 1C.

Large volume apheresis started on the 4th day of G-CSF administration, which was generally day −3 relative to CD34-enriched (HSPC) and Treg product infusions into the subject (defined as Day 0). Apheresis commenced with a target of ≥3×10⁶ CD34⁺ cells/kg recipient body weight post-selection. In order to have sufficient cells available for CD34 enrichment and Treg sorting procedures, donors underwent apheresis collections on 2 consecutive days (e.g., Days −3 and −2). To the degree possible, the 1st day's apheresis (Day −3) collection was scheduled for afternoon hours and the 2nd day's (Day −2) for early morning, thereby limiting the time from the end of the first collection to the infusion of the cellular products to less than 72 hours. Plerixafor (e.g., 0.24 mg/kg SC, once) was available prior to the second apheresis if recommended by the investigator and/or attending physician to achieve the HSPC dose target.

The CD34 reduced (flow-through) fractions were retained and used for isolation of donor Treg. For cell selection, clinical grade reagents were used under Good Manufacturing Practice (GMP) Conditions within the BMT Cellular Therapy Facility.

CD25⁺ cells were then selected from the CD34-depleted fraction using bead purification (Miltenyi). Tcons were obtained from the negative fraction and the positive fraction was used for Treg purification. CD4⁺CD25⁺CD127dim cells underwent further selection by FACS using a BD Influx cell sorter (BD Biosciences, San Jose CA). Enrichment of Tregs was following depletion of CD34⁺ cells by immunomagnetic selection, selection of CD25⁺ by immunomagnetic selection and purification by FACS sorting of CD4⁺ CD127lowCD25⁺ cells. High purity of Tregs were obtained. These cells were highly suppressive in a mixed lymphocyte reaction (MLR).

When a lower than intended Treg dose level was achieved, e.g., a final Treg yield of <2×10⁶/kg body weight of the recipient, the recipient received a dose of 1-2×10⁶ Treg/kg if that dose could be achieved, and a reduced Tcon dose such that the ratio of administered Treg to Tcon was 1:1.

### E. Treatments

Subjects received the cell components indicated in TABLE 9

TABLE 9

| | | Cell Population |
|---|---|---|
| | | Target Dose Range, |
| Graft Component: | Dose Regimen | per Recipient kg |
| HSPC (also described herein | IV, Once, on Day 0 | ≥3 × 10⁶ |
| as a “first composition of | | |
| CD45⁺ cells) | | |
| Treg (also described herein as | IV, Once, on Day 0 | 2-3 × 10⁶ |
| a “cell composition enriched | | |
| for Tregs) | | |
| Tcon (also described herein as | IV, Once, on Day ⁺2 | 3 × 10⁶ |
| a “second composition of | | |
| CD45⁺ cells) | | |

In the Examples, term “HSPC” as a transplant, as a graft, as a cell dose, as a product, as a drug product, as a component, or as a graft component, and the like corresponds to the “first composition of CD45⁺ cells” and the like as disclosed elsewhere in the application, including the claims; the term “Treg” as a transplant, as a graft, as a cell dose, as a product, as a drug product, as a component, or as a graft component, and the like corresponds to the “cell composition enriched for Tregs) and the like as disclosed elsewhere in the application, including the claims; and the term “Tcon” as a transplant, as a graft, as a cell dose, as a product, as a drug product, as a component, or as a graft component, and the like corresponds to the “second composition of CD45⁺ cells) and the like as disclosed elsewhere in the application, including the claims.

Patients received Treg cells with purity higher than 90%, with a median Treg purity of 93.8%+/−3.1%. FIGS. 2B-D illustrates the cell dose of HSPCs and Treg cells administered to patients. Table 10 below illustrates an analysis of the HSPC drug product (e.g., the first population of CD45⁺ cells) from a representative subset of 20 subjects enrolled in this study. Table 11 below illustrates an analysis of the Treg drug product (e.g., the population of cells enriched for Tregs) from a representative subset of 20 subjects enrolled in this study. Cell components were provided as single dose transfer bags, with an approximate fill volume of 100 mL each for both the Treg and the HSPC components. For each one of Tables 10-11, samples from 20 subjects from the multicenter clinical trial were analyzed. Means and standard deviations (SD) shown in each table. The mean (SD) patient weight was 71.8 kg (11.2 kg). Impurity cell doses were calculated by multiplying the % impurity by the reported cell dose for each patient.

The Tcon component (e.g., the third population of CD45⁺ cells) was provided frozen, after storage in a vapor phase liquid nitrogen tank, in an approximate volume of 15 mL. The pooled apheresis product was assessed for CD3⁺ Tcon cells and a volume of the apheresis frozen product comprising 3.0E⁺06 Tcons was calculated and administered to subjects. Table 12 below illustrates an analysis of the Tcon drug product from a representative subset of 20 subjects enrolled in this study. Analysis of the Tcon drug products from 20 grafts in the multicenter clinical trial (N=20). Means and standard deviations (SD) shown. Cell doses were calculated for the cell populations based on the targeted CD3⁺ T cell dose of 3M/kg. Biomarker data (BM), manufacturing data (mfg); mean (SD) patient weight was 71.8 kg (11.2 kg).

Each cellular therapy product comprises cells, Plasma-Lyte A, and human serum albumin, at a pH of approximately 7.4. The bags and/or primary bag containers had labels bearing the appropriate label text as required by governing regulatory agencies.

The amount of time from when the cell product was received from the donor to when it was administered to the recipient was under 60 hours as is shown for a subset of patients in TABLE 13.

Dosing was based on a subject's actual body weight rounded to the nearest tenth of a kilogram, as assessed during screening, or based on adjusted body weight (ABW) if the subject's actual weight is greater than 120% of the ideal body weight (IBW), calculated as ABW [in kg]=[(actual weight−IBW)×0.40]⁺IBW.

TABLE 14 illustrates endotoxin levels in each cell population for a subset of patients.

TABLE 10
HSPC product analysis

| | % of Live | | Cell Dose/ | |
|---|---|---|---|---|
| Cell population | CD45⁺ cells | SD | kg | SD |
| CD45⁺ live cells | 100 | 0.0 | 8.2E⁺06 | 3.6E⁺06 |
| HSPCs (purity) | 93.3 | 11.5 | 7.7E⁺06 | 3.8E⁺06 |
| Non-HSPCs (impurity) | 6.7 | 11.5 | 4.2E⁺05 | 7.3E⁺05 |
| Granulocytes | 3.2 | 6.8 | 1.9E⁺05 | 4.3E⁺05 |
| Monocytes | 1.2 | 2.2 | 7.2E⁺04 | 1.3E⁺05 |
| Lymphocytes | 1.1 | 0.8 | 7.8E⁺04 | 5.7E⁺04 |
| Non-T lymphs | 1.0 | 0.8 | 7.2E⁺04 | 5.6E⁺04 |
| T cells | 0.1 | 0.1 | 6.1E⁺03 | 9.5E⁺03 |

TABLE 11
Treg Product analysis

| | % of Live | | Cell | |
|---|---|---|---|---|
| Cell population | CD45⁺ cells | SD | Dose/kg | SD |
| CD45⁺ live cells | 100.0 | 0.0 | 2.8E⁺06 | 3.6E⁺05 |
| Tregs (purity) | 96.5 | 2.2 | 2.7E⁺06 | 3.7E⁺05 |
| Non-Tregs (impurity) | 3.5 | 2.2 | 9.3E⁺04 | 5.4E⁺04 |
| Grans and Monos | 0.1 | 0.1 | 1.9E⁺03 | 1.4E⁺03 |
| Lymphocytes | 3.2 | 2.1 | 8.6E⁺04 | 5.2E⁺04 |
| T cells | 2.4 | 1.9 | 6.3E⁺04 | 4.6E⁺04 |
| CD4⁺ T cells | 2.3 | 1.9 | 6.2E⁺04 | 4.6E⁺04 |
| Non CD4⁺ T cells | 0.0 | 0.1 | 1.4E⁺03 | 1.6E⁺03 |
| Non-T cell lymphs | 0.8 | 1.2 | 2.2E⁺04 | 3.2E⁺04 |

TABLE 12
Tcon product analysis

| | % of Live | | Cell | |
|---|---|---|---|---|
| Cell population | CD45⁺ cells | SD | Dose/kg | SD |
| CD45⁺ live cells (mfg) | 100.0 | 0.0 | 1.0E⁺07 | 2.4E⁺06 |
| CD45⁺ cell (BM) | 100.0 | 0.0 | 1.9E⁺07 | 7.1E⁺06 |
| Grans⁺Monos (mfg) | 52.2 | 8.1 | 6.2E⁺06 | 2.3E⁺06 |
| Grans⁺Monos (BM) | 56.7 | 8.8 | 1.1E⁺07 | 5.8E⁺06 |
| Granulocytes (BM) | 27.6 | 9.9 | 5.6E⁺06 | 3.9E⁺06 |
| Monocytes (BM) | 25.7 | 6.6 | 4.9E⁺06 | 2.1E⁺06 |
| Lymphocytes (mfg) | 41.1 | 7.8 | 4.6E⁺06 | 5.7E⁺05 |
| Lymphocytes (BM) | 29.8 | 9.4 | 5.1E⁺06 | 6.8E⁺05 |
| CD3⁺ (T) cells reported | 30.1 | 6.4 | 3.0E⁺06 | 0.0E⁺00 |
| CD3⁺ (T) cells (BM) | 17.7 | 5.7 | 3.0E⁺06 | 0.0E⁺00 |
| CD4⁺T cells (BM) | 8.8 | 3.5 | 1.5E⁺06 | 3.2E⁺05 |
| CD8⁺ T cells (BM) | 7.4 | 3.2 | 1.2E⁺06 | 3.0E⁺05 |
| B cells (BM) | 5.2 | 2.5 | 9.1E⁺05 | 3.7E⁺05 |
| NK cells (BM) | 3.6 | 1.9 | 6.4E⁺05 | 3.3E⁺05 |
| HSPCs (mfg) | 0.8 | 0.4 | 9.0E⁺04 | 5.8E⁺04 |
| Tregs (BM) | 0.5 | 0.2 | 8.2E⁺04 | 3.8E⁺04 |

TABLE 13
Time duration between collection and administration

| Category | Activity | N | Time (hrs) |
|---|---|---|---|
| Transportation | Local collection site | N = 17 | 2.8 ⁺/− 1.3 |
| | to sorting facility | | |
| Transportation | Out of State | N = 7 | 11.2 ⁺/− 2.6 |
| | collection facility to | | |
| | sorting facility | | |
| Manufacturing | Product dwell time at | N = 24 | 25.6 ⁺/− 5.0 |
| | sorting facility | | |
| Total | Vein-to-vein | N = 24 | 60.5 ⁺/− 10.9 |

TABLE 14
Endotoxin levels

| | Endotoxin | Endotoxin | Endotoxin |
|---|---|---|---|
| | (EU/ml) in HSPC | (EU/ml) in Treg | (EU/ml) in Tcon |
| Patients | drug product | drug product | drug product |
| 1 | <0.025 | <0.05 | <0.5 |
| 2 | <0.025 | <0.05 | <0.5 |
| 3 | 0.043 | <0.061 | <0.5 |
| 4 | <0.025 | <0.061 | <0.5 |
| 5 | <0.026 | <0.05 | <0.5 |
| 6 | <0.025 | <0.05 | <0.5 |
| 7 | <0.025 | <0.05 | <0.5 |
| 8 | <0.025 | <0.05 | <0.5 |
| 9 | <0.025 | <0.05 | <0.5 |
| 10 | <0.025 | <0.05 | <0.717 |
| 11 | <0.025 | <0.05 | <0.5 |
| 12 | <0.025 | <0.05 | <0.5 |
| 13 | <0.025 | <0.05 | <0.5 |
| 14 | <0.025 | <0.05 | <0.5 |
| 15 | <0.025 | <0.05 | <0.5 |
| 16 | <0.025 | <0.05 | <0.5 |
| 17 | <0.025 | <0.05 | <0.5 |
| 18 | <0.025 | <0.05 | <0.5 |
| 19 | <0.025 | <0.05 | <0.5 |
| 20 | <0.025 | <0.05 | <0.5 |
| 21 | <0.025 | <0.05 | <0.5 |
| 22 | <0.025 | <0.05 | <0.5 |
| 23 | <0.025 | <0.05 | <0.5 |
| 24 | <0.025 | <0.05 | <0.5 |
| 25 | <0.025 | <0.05 | <0.5 |
| 26 | <0.025 | <0.05 | <0.5 |
| 27 | <0.025 | <0.05 | <0.5 |
| 28 | <0.025 | <0.05 | <0.5 |
| 29 | <0.5 | <0.5 | <0.5 |
| 30 | <0.025 | <0.05 | <0.5 |
| 31 | <0.025 | <0.05 | <0.5 |
| 32 | <0.025 | <0.05 | <0.5 |
| 33 | <0.025 | <0.05 | <0.5 |
| 34 | <0.025 | <0.063 | <0.5 |
| 35 | <0.025 | <0.05 | <0.5 |
| 36 | <0.025 | <0.05 | <0.5 |
| 37 | <0.025 | <0.05 | <0.5 |
| 38 | <0.025 | <0.05 | <0.5 |
| 39 | <0.025 | <0.05 | <0.5 |
| 40 | <0.025 | <0.05 | <0.5 |
| 41 | <0.025 | <0.05 | <0.5 |
| 42 | <0.025 | <0.05 | <0.5 |
| 43 | <0.025 | <0.05 | <0.5 |
| 44 | <0.025 | 0.066 | <0.636 |
| 45 | <0.025 | 0.05 | <0.5 |
| 46 | <0.025 | 0.05 | <0.5 |
| 47 | <0.025 | <0.05 | <0.5 |
| 48 | <0.025 | <0.025 | <0.5 |
| 49 | <0.025 | 0.075 | <0.5 |
| 50 | <0.025 | <0.05 | <0.5 |
| 51 | <0.025 | <0.05 | <0.5 |
| 52 | <0.025 | <0.05 | <0.5 |
| 53 | <0.025 | <0.05 | <0.5 |
| 54 | <0.0391 | <0.05 | <0.5 |
| 55 | <0.025 | <0.05 | <0.5 |
| 56 | <0.025 | <0.05 | <0.5 |
| 57 | <0.025 | <0.05 | <0.5 |
| 58 | <0.025 | <0.05 | <0.809 |
| 59 | <0.0294 | <0.05 | <0.5 |
| 60 | 0.0457 | <0.05 | <0.5 |
| 61 | 0.0427 | <0.063 | <0.5 |
| 62 | <0.025 | <0.05 | <0.5 |

Recipients had appropriate long-term central venous access placed prior to initiation of the conditioning regimen. Unless contraindicated, subjects were administered acetaminophen or paracetamol (e.g., 500-1000 mg) and diphenhydramine (e.g., 25-50 mg) prior to administration of each cell component.

The CD34⁺ HSPC cell component (e.g., the first population of CD45⁺ cells), then the Treg cell component (e.g., the population of cells enriched for Tregs), were intravenously (IV) through a central venous catheter on study Day 0.

Subjects received a myeloablative conditioning (MA) regimen prior to administration of the cell components. Examples of MA regimens are provided in TABLE 15. Busulfan could also be dosed to maintain an average daily AUC of 4,800-6,000 μM-min.

TABLE 15

| MA regimen | Drugs and doses |
|---|---|
| Total Body Irradiation/ | TBI (1320-1400 cGy) |
| Cyclophosphamide/ | Cyclophosphamide (120 mg/kg) |
| Thiotepa (TBI/Cy/Thiotepa) | Thiotepa (10 mg/kg) |
| Busulfan/Melphalan/ | Busulfan (9.6 mg/kg IV) |
| Fludarabine | Fludarabine (125 mg/m²) |
| (Bu/Mel/Flu) | Melphalan (140 mg/m²) |
| Busulfan/Fludarabine | Busulfan (16 mg/kg PO or |
| (Bu/Flu) | 12.8 mg/kg IV) |
| | Fludarabine (160 mg/m²) or |
| | 120-180 mg/m² administered |
| | over 4 days |
| Busulfan/Cyclophosphamide | Busulfan (16 mg/kg PO or |
| (Bu/Cy) | 12.8 mg/kg IV) |
| | Cyclophosphamide (100 mg/kg) |
| Cyclophosphamide/Total | Cyclophosphamide (100 mg/kg) |
| Body Irradiation | TBI (1200-1420 cGy) |
| (Cy/TBI) | |
| Total Body Irradiation/ | TBI (1200-1320 cGy) |
| Etoposide | Etoposide (60 mg/kg) |
| (TBI/Etoposide) | |
| Total Body Irradiation/ | TBI (1320 cGy) |
| Etoposide/Cyclophosphamide | Etoposide (60 mg/kg, 1 dose) |
| (TBI/Etoposide/Cy) | Cyclophosphamide (60 mg/kg, 1 dose) |
| Bu/Flu/TTP | Thiotepa 5 mg/kg × 2 days |
| | (days −7 and −6) |
| | Busulfan 3.2 mg/kg × 3 days |
| | (days −5 to −3), |
| | total dose 9.6 mg/kg |
| | Fludarabine 50/m² (meters |
| | squared − body surface |
| | area) × 3 days (days −5 to −3) |
| TBI/Flu/TTP | Total body irradiation: HFTBI |
| | administered in 11 fractions of |
| | 125 cGy (centigray) over 4 |
| | days, to a total dose of 1375 cGy. |
| | Thiotepa 5 mg/kg × 2 days |
| | (days −7 and −6) |
| | Fludarabine 25/m² × 5 days |
| TBI/Flu/TTP/Bu | Thiotepa 5 mg/kg × 2 days |
| | (days −7 and −6) |
| | Busulfan 3.2 mg/kg × 3 days |
| | (days −5 to −3), total |
| | dose 9.6 mg/kg |
| | Fludarabine 50/m² (meters |
| | squared − body |
| | surface area) × 3 days |
| | (days −5 to −3) |
| | Total body irradiation: HFTBI |
| | administered in 11 fractions of |
| | 125 cGy (centigray) over 4 days, |
| | to a total dose of 1375 cGy |

Subjects received either tacrolimus or sirolimus as a single-agent GVHD prophylaxis beginning on the day following Tcon infusion (typically Day ⁺3), e.g., of the third population of CD45⁺ cells.

Tacrolimus was initiated at 0.03 mg/kg/day IV, with a target trough blood level of 5-10 ng/mL. Per os (PO) administration was permissible if the patient was able to tolerate food.

For subjects that underwent MA with TBI/Cy/Thiotepa, Cy/TBI, TBI/Etoposide, TBI/Etoposide/Cy (i.e., regimens that do not include busulfan), sirolimus was initiated as a single loading dose of 6 mg PO, followed by 2 mg daily for a target blood level of 3-8 ng/ml.

For subjects that did not show signs of ≥grade 2 acute GVHD prior to Day ⁺60, the GVHD prophylaxis could be reduced, e.g., by approximately 20% of the dose per month. For subjects that showed signs of ≥grade 2 acute GVHD, GVHD prophylaxis could be tapered after no signs of GVHD were observed for a suitable period of time (e.g., a suitable period of time after ceasing administration of any GVHD therapeutic agents, and not observing ≥grade 2 GVHD).

### F. Clinical Outcomes

The following paragraphs provide data and explanations related to clinical outcomes from the clinical studies described herein. As an introductory matter in the explanation of the data, it is to be recognized that graft failure can be a complication of HCT, and can be associated with significant mortality.

Neutrophil engraftment through Day ⁺28: Neutrophil engraftment was defined as achieving an absolute neutrophil count (ANC)≥500/mm³ for 3 consecutive days, by Day ⁺28. The first of the three days was designated the day of engraftment. If ANC never dropped below 500/mm³, Day ⁺1 was assigned as the day of engraftment. Study group patients showed earlier neutrophil (median of 11 days vs. 14 days, p<0.0001 by Mann-Whitney U).

Platelet engraftment through Day ⁺50: Platelet engraftment was defined as achieving a platelet count >20,000/mm³ for 3 consecutive days without platelet transfusion in the preceding 7 days, by Day ⁺50. The first of the three days was designated the day of engraftment. If platelet count never dropped below 20,000/mm³, Day ⁺1 was assigned as the day of engraftment. Study group patients showed earlier platelet engraftment (11 vs 17 days, p<0.0001).

Secondary graft failure through Day ⁺100: Secondary graft failure was defined as neutrophil engraftment followed by subsequent decline in absolute neutrophil counts <500 cells/uL, unresponsive to growth factor therapy, by Day ⁺100.

A failure to achieve an absolute neutrophil count of >500 cells/μL after Day ⁺30 can indicate primary graft failure. No subjects in the study group experienced primary graft failure.

A sustained loss of hematopoiesis after engraftment has occurred can indicate secondary graft failure. No subjects in the study group experienced secondary graft failure.

Peripheral blood samples from subjects were also analyzed for chimerism as part of the follow-up procedures. The blood samples were processed to select for various cell populations using standard markers for each cell population. Blood was drawn from subjects that received the composition of the disclosure on day ⁺28, day ⁺56, day ⁺100, day ⁺180 and day ⁺365 post-transplant, and the frequency of peripheral blood cells were quantified by flow cytometry (Markers used for analysis are described in Table 16).

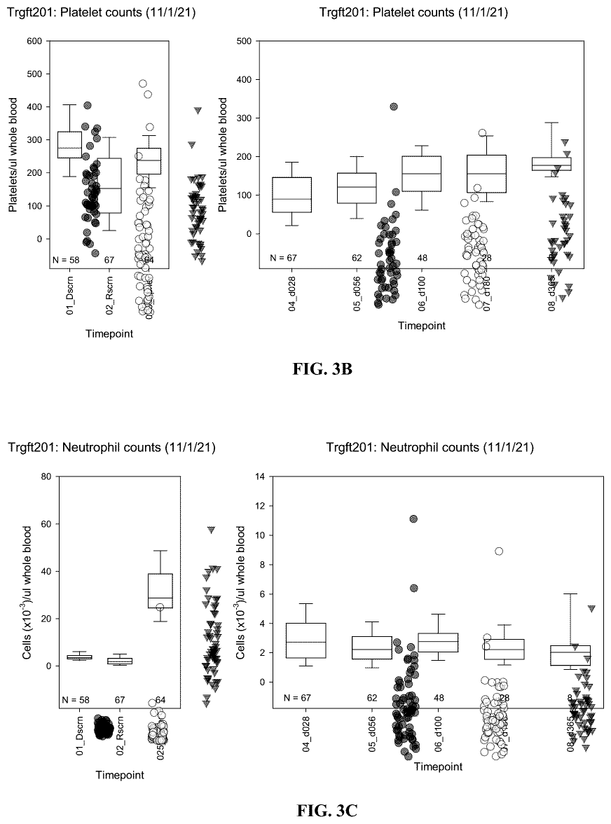

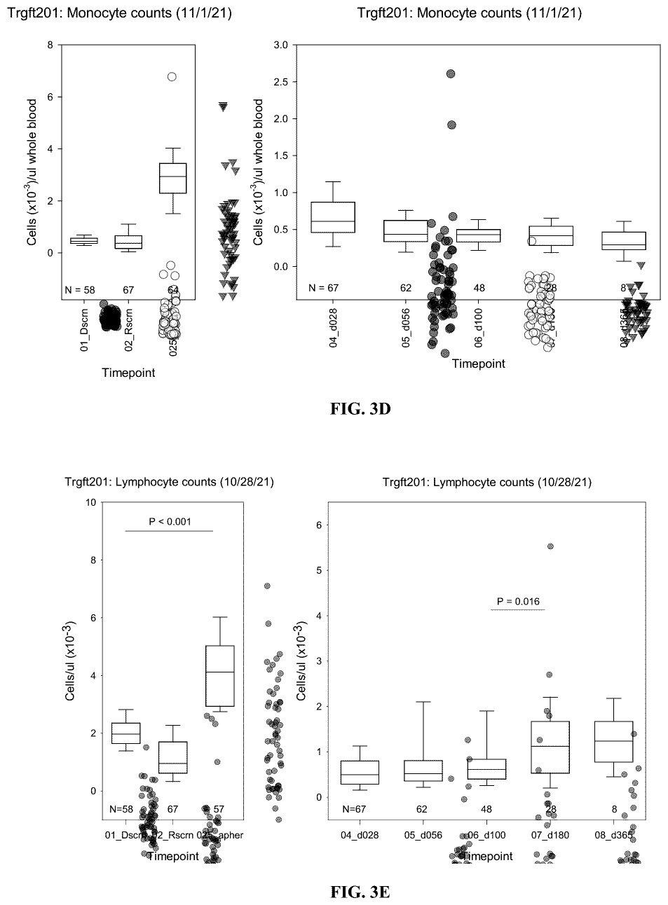

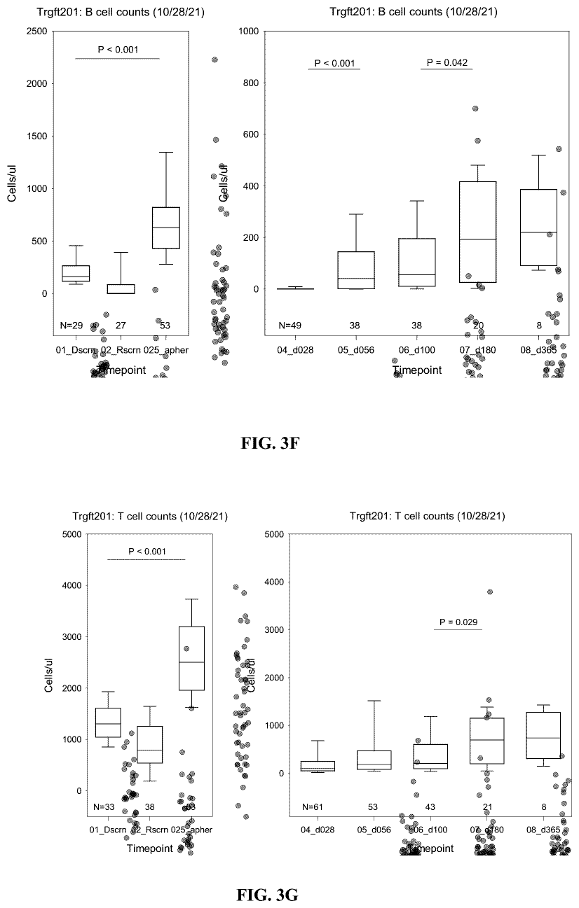

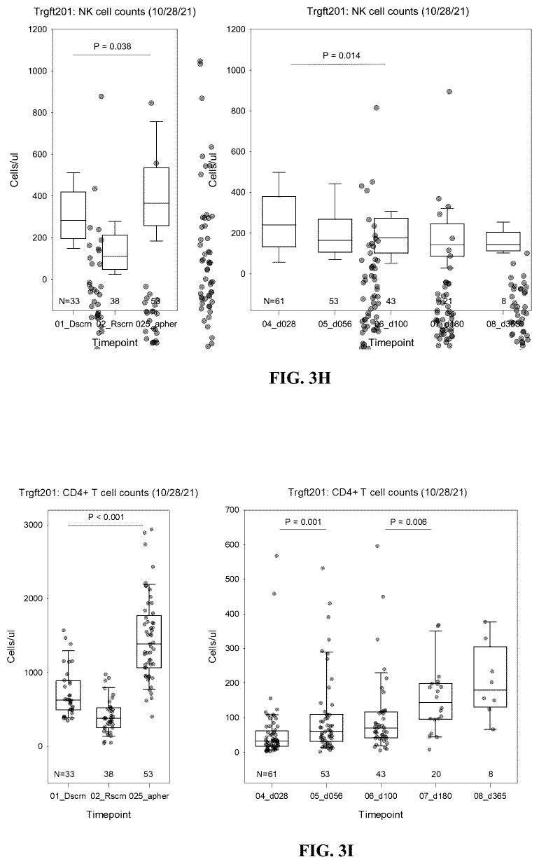

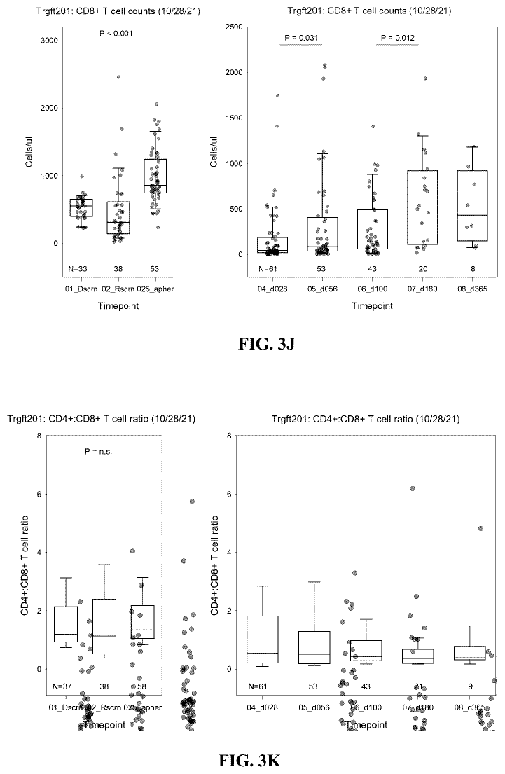

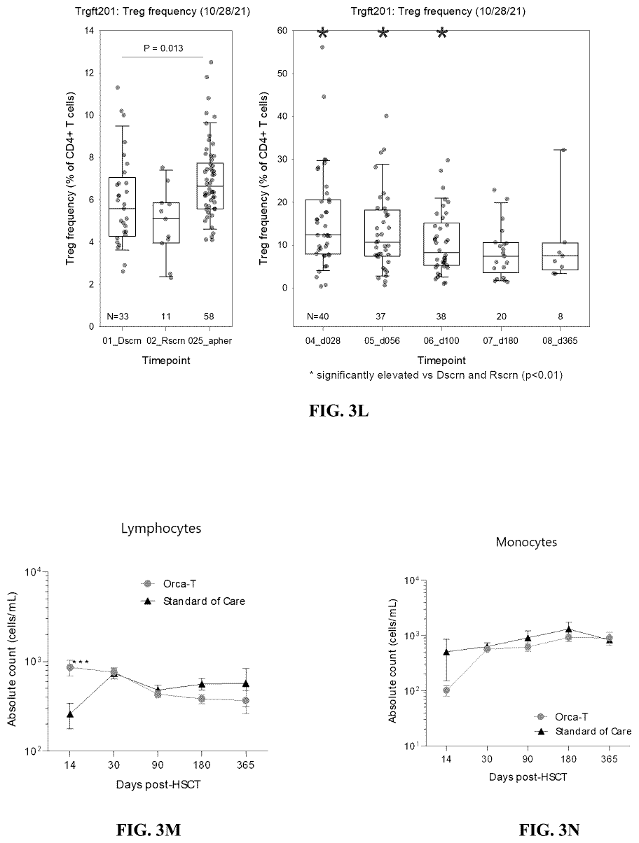

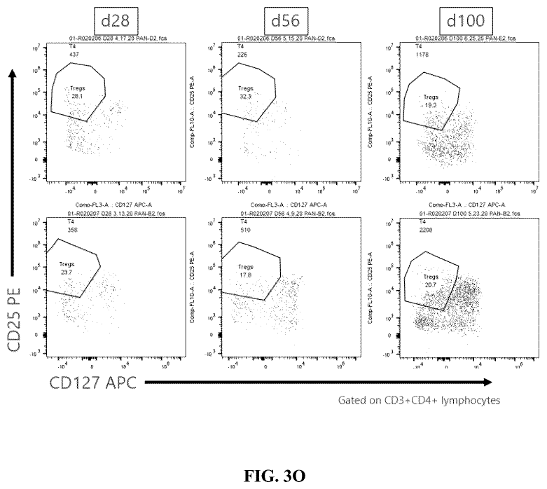

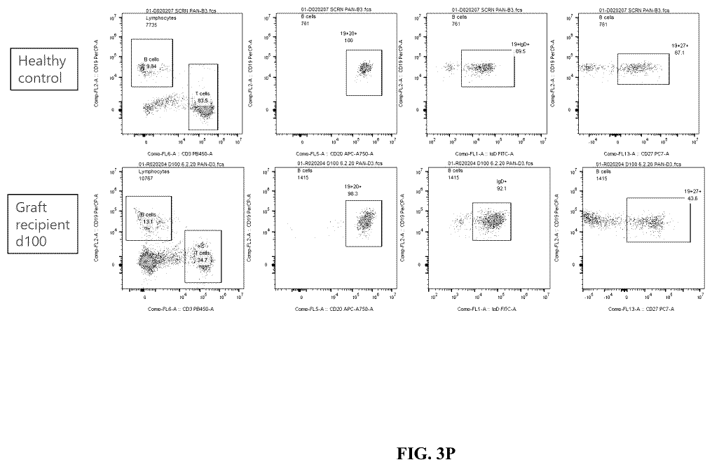

Achieving a platelet count >20,000/mm³ for 3 consecutive days without platelet transfusion can indicate platelet engraftment. As shown in FIG. 3A, subjects in the study group exhibited more rapid platelet engraftment than subjects in the SOC cohort, achieving platelet engraftment after a median of 11 days compared to 17 days for the SOC cohort (p<0.0001). FIG. 3B illustrates the platelet counts in donors before and after mobilization and in receiving patients before transplant and after transplants. Boxplots where shown: boxes show the 75^{th}, 50^{th}, and 25^{th} percentiles; whiskers show the 90^{th} and 10^{th} percentiles. X-axes nomenclature: the leading number (e.g., 01, 02, 025, . . . ) are mentioned for ordering; following the underscore, Dscrn=healthy donor pre-G-CSF mobilization, Rscrn=recipient within 1 month prior to conditioning, apher=healthy donor blood draw at the time of apheresis, d028=recipient day 28 post-transplant, d056-d365=recipient days post-transplant. N's shown indicate the sample sizes for each timepoint. Symbols indicate values for individual measurements.

Achieving a sustained neutrophil count of >500 cells/μL can indicate neutrophil engraftment. FIG. 3C illustrates the platelet counts in donors before and after mobilization and in receiving patients before transplant and after transplants. Figure legends are similar to the legend described in FIG. 3B.

FIGS. 3D-E illustrate monocyte and lymphocyte engraftment in the patients. FIGS. 3M and N illustrate a comparison of lymphocyte and monocyte counts in representative patients compared to a representative SOC cohort.

FIG. 3F illustrates that B cells engraft in recipients of the composition of the disclosure, and that mature B cells are present by day ⁺100 post-transplant. Superior engraftment and/or earlier functionality of engrafted B cells may represent a significant advantage over standard of care grafts, for example, enhancing immunity and allowing for vaccination post-transplant.

FIG. 3G illustrates CD3⁺ T cell engraftment; FIG. 3H demonstrates NK cell engraftment, FIG. 3I illustrates CD4⁺ T cell engraftment and FIG. 3J illustrates CD8⁺ T cell engraftment in recipients of the composition of the disclosure. FIG. 3K illustrates a ratio of CD4:CD8 T cells in the recipients.

FIG. 3L provides a comparison of the proportion of CD3⁺ CD4⁺ T cells that were Tregs in healthy donors, compared to graft recipients on several days post-transplant. These data show that recipients of the composition of the disclosure exhibit high frequencies of circulating CD4⁺ Tregs. Without wishing to be bound by theory, a lower Treg frequency may contribute to prevention of GVHD, reduced risk of developing GVHD, reduced incidence of GVHD, reduced severity of GVHD, or a combination thereof.

FIG. 30 shows representative data from two subjects compared to a healthy control. In the healthy control, 3.72% of circulating CD3⁺CD4⁺ T cells were Tregs (CD25⁺ CD127^{dim}). In the two graft recipients, 28.1% and 23.7% of CD3⁺CD4⁺ T cells were Tregs on day ⁺28, 32.3% and 17.8% on day ⁺56, and 19.2% and 20.7% on day ⁺100.

Blood drawn from a graft recipient on day ⁺100 post-transplant the sample was processed for flow cytometric evaluation of various B cell markers including CD19, CD20, IgD, and CD27. FIG. 3P compares scatterplots from the graft recipient to a healthy control. In all cases, the Y axis is for CD19⁺ staining. The left panels show gating of lymphocytes to identify B cells (CD19⁺) and T cells (CD3⁺). 13.4% of lymphocytes in the graft recipient were B cells, compared to 9.84% in the healthy control. The following panels show that 98.3-100% of cells gates as CD19⁺ were also CD20⁺. The panels second from the right show the fraction of B cells that are IgD⁺, which can be used to identify mature B cells. 92.1% of B cells in the graft recipient were IgD⁺, and 89.5% in the healthy control. The right-most panels show staining for CD27, which can be used to identify memory B cells, late plasmablasts, and plasma cells, for example. 43.6% of B cells in the graft recipient were CD27⁺, and 67.1% in the healthy control.

These results demonstrate that B cells engraft in recipients of the composition of the disclosure, and that mature B cells are present by day ⁺100 post-transplant. Superior engraftment and/or earlier functionality of engrafted B cells may represent a significant advantage over standard of care grafts, for example, enhancing immunity and allowing for vaccination post-transplant.

### G. GVHD Evaluation

Acute GVHD was staged and graded per MAGIC Standardization criteria.

Chronic GVHD was diagnosed, staged, and graded per the International NIH Chronic GVHD Diagnosis and Staging Consensus Working Group criteria.

Clinically significant manifestations of both acute and chronic GVHD were treated first by local, topical, and/or systemic corticosteroids (e.g., prednisone). Any patients who were refractory or resistant to, dependent upon, or intolerant of corticosteroids, per BMT-NIH-CIBMTR Task Force definition, were to be considered for second line therapy.

Treatment-emergent adverse events (TEAEs): TEAEs were categorized by System Organ Class and preferred term using MedDRA version 21.0 and were graded according to the CTCAE version 5.0.

Acute GVHD: Acute GVHD (aGVHD) is a significant driver of morbidity and mortality associated with alloHSCT, reducing the severity and incidence of aGVHD has the potential to greatly benefit graft recipients. Acute GVHD was staged and graded per Mount Sinai Acute GvHD International Consortium (MAGIC) Standardization criteria.

Subjects were considered evaluable for aGVHD if they developed aGVHD symptoms before day ⁺100 (100 days post-transplant), or were beyond day ⁺100 without exhibiting aGVHD symptoms. Using these criteria, 17 patients are evaluable for aGVHD at the time of reporting.

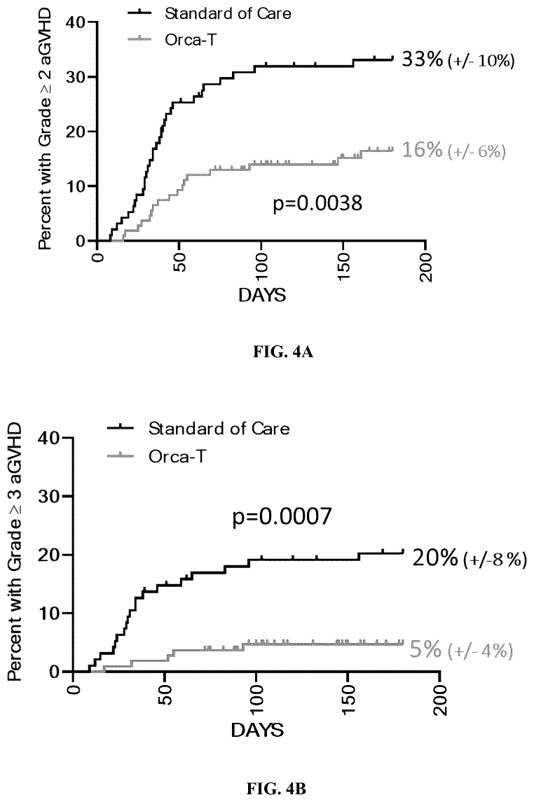

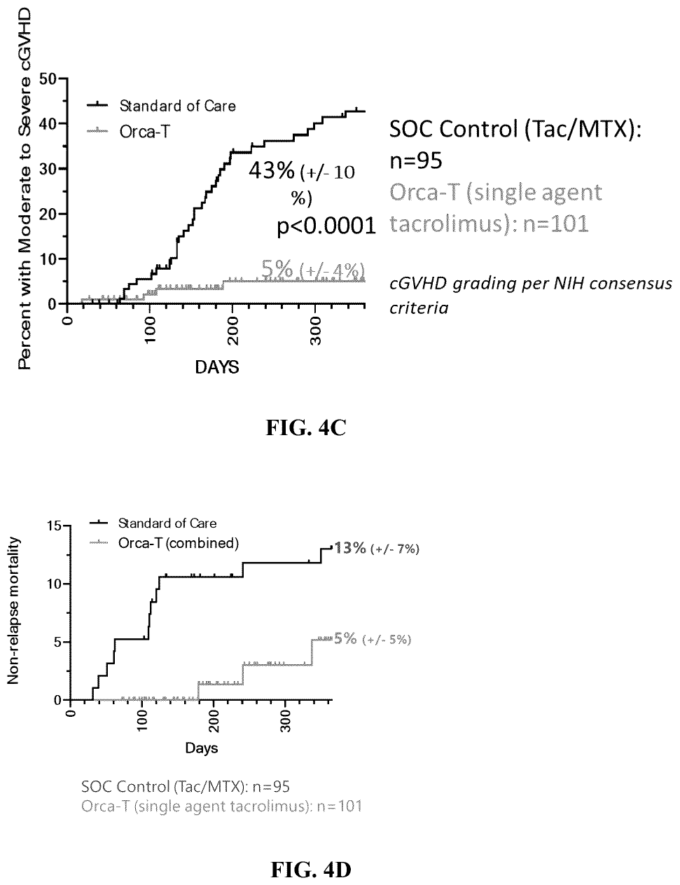

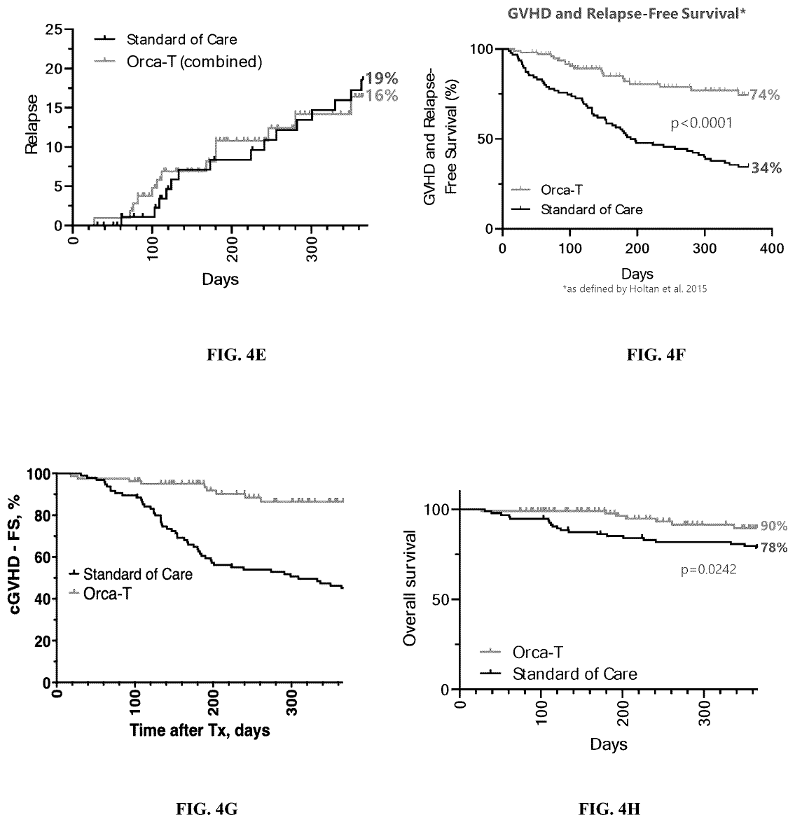

The Grade ≥2 aGVHD rate observed was 16% at the time of reporting. This compares favorably to published rates of in similar populations, and was lower than patients in the SOC cohort. The onset of grade ≥2 aGVHD compared to the SOC cohort is shown in FIG. 4A. One subject developed aGVHD of the upper gastrointestinal (GI) tract, manifesting as nausea and cachexia. This subject's symptoms resolved with a short course of corticosteroids, which were subsequently weened.

Severe (Grade 3-4) aGVHD is a major contributor to non-relapse mortality post-alloHSCT and can be observed in 10-20% of patients following an HLA-matched, related donor transplants. 5% of the patients developed grade 3-4 aGVHD in the study group, whereas 20% of patients in the SOC cohort developed Grade 3-4 aGVHD. The onset of grade ≥3 aGVHD in through Day ⁺120 compared to the SOC cohort is shown in FIG. 4B.

As noted in TABLE 7, four patients in the study group did not receive any GVHD prophylaxis. Of those patients, only one has developed aGVHD symptoms (the case of upper GI tract aGVHD noted above). Results to the time of reporting suggest that the composition of the disclosure may be safely administered to patients without GVHD prophylaxis or with minimal prophylaxis. These strategies could significantly benefit patients due to, for example, increased graft versus tumor (GVT), increased graft versus infection (GVI), and reduced adverse effects that can be associated with immunosuppressive agents (e.g., renal toxicity and hepatotoxicity).

These results suggest a composition of the disclosure can reduce the incidence and severity of aGVHD in recipients compared to standard of care, for example, even in the absence of GVHD prophylactics or with reduced GVHD prophylaxis.

Steroid-refractory acute GVHD: Steroid-refractory acute GVHD was defined as per the EBMT-NIH-CIBMTR Task Force position statement.

Chronic GVHD: As chronic GVHD (cGVHD) is associated with significant morbidity and with decreased survival, reducing the severity or incidence of cGVHD has the potential to greatly benefit graft recipients. Chronic GVHD was diagnosed per 2014 International NIH Chronic GVHD Diagnosis and Staging Consensus Working Group criteria.

At the time of reporting, no subjects in the study group had developed moderate or severe cGVHD. One subject in the study group developed transient, steroid-responsive mild cGVHD of the skin.

Analysis was conducted using a cutoff of 365 days (i.e., cGVHD events that occur after Day ⁺365 were not included). As shown in FIG. 4C, Subjects in the study group experienced significantly fewer moderate or severe cGVHD events than patients in the SOC cohort (5% vs. 43%, respectively; p<0.0001). Despite receiving less GVHD prophylaxis or no GVHD prophylaxis, data showed that a composition of the disclosure can significantly reduce the incidence of cGVHD compared to subjects in the SOC cohort. Additionally, several subjects in the study group have undergone greater than 2.5 years of follow-up with no cGVHD symptoms reported.

As cGVHD is associated with decreased overall survival and significant long-term morbidity, these results suggest that a composition of the disclosure can improve long-term survival and quality of life in recipient subjects compared to standard of care.

Post-Transplant Lymphoproliferative Disorder (PTLD): PTLD was defined as a biopsy consistent with the 2017 World Health Organization (WHO) classification of PTLD (nondestructive [plasmacytic hyperplasia, infectious mononucleosis-like, and florid follicular hyperplasia], polymorphic, monomorphic or Hodgkin lymphoma-like), along with lymphoma type-appropriate staging procedures such as computed tomography (CT) with or without 18F-fluorodeoxyglucose positron-emission tomography (FDG-PET).

Incidence of non-relapse mortality (NRM). NRM was defined as death without evidence of disease recurrence. Disease relapse/progression was considered a competing event.

Incidence of disease relapse: For acute leukemias, relapse was defined as any of the following (MRD⁺ alone was insufficient): (i) ≥5% blasts in the bone marrow or peripheral blood; or (ii) Reappearance of pre-transplant cytogenetic abnormality; or (iii) new evidence or redevelopment of extramedullary disease. For MDS, relapse was defined as any of the following: (i) satisfying criteria for evolution into acute leukemia; (ii) reappearance of pre-transplant morphologic abnormalities, detected in bone marrow specimens; or, (iii) reappearance of pre-transplant cytogenetic abnormality in at least one metaphase on each of two separate consecutive examinations at least one month apart, regardless of the number of metaphases analyzed.

Treatment related mortality includes deaths from complications or toxicities associated with therapy, such as infection, GVHD, or organ failure. At the time of reporting, the treatment-related mortality rate in the study group was 5% for one-year post-transplant, compared to 13% for the SOC cohort, as shown in FIG. 4D. No subjects in the study group had died from complications other than disease relapse.

Subjects were monitored for survival and relapse. For comparison of clinical outcomes, a standard of care (SOC) comparator cohort was identified retrospectively from subjects who received a SOC myeloablative alloHSCT of mobilized peripheral blood from a fully HLA-matched donor at one of the same clinical sites during the same period. All subjects in the SOC cohort received myeloablative conditioning regimens and dual-agent GVHD prophylaxis with tacrolimus and methotrexate.

FIGS. 4E-H illustrate the relapse, GVHD and relapse free survival rates, chronic GVHD free survival rates and overall survival rates of subjects that were recipients of standard of care grafts compared to subjects that received grafts described in this example. These data suggest that the compositions described herein improve relapse-free survival in subjects undergoing myeloablative alloHSCT.

GVHD and relapse-free survival is a composite readout that can refer to survival without relapse or Grade ≥3 acute or extensive chronic GVHD. At the time of reporting, the percent of GVHD-free and relapse-free survival to one-year post-transplant was 74% for the study group, compared to 34% for the SOC cohort, as shown in FIG. 4F. The difference was statistically significant (p=0.0001 by log-rank test).

Overall survival: OS was defined as the time from the date of transplant to the date of death from any cause or, for surviving patients, to the date of last follow-up. At the time of reporting, the percent of overall survival to one-year post-transplant was 90% for the study group, compared to 78% for the SOC cohort, as shown in FIG. 4H. The difference was statistically significant (p=0.0242 by log-rank test).

### H. Hospitalization Time

Standard of care HCT can require a lengthy inpatient stay. Compared to patients in the SOC cohort, the median time from transplant (Day 0) to hospital discharge for patients in the study group was 2.5 days shorter (from 18.5 to 16 days, p<0.01 by Mann-Whitney U test). FIG. 4I illustrates hospitalization times for a representative subset of patients.

I. Disease Status after Therapy

Subjects in the study were first evaluated for relapse on day ⁺90 post-transplant. At the time of reporting, only 16% patients relapsed compared to 19% patients in the SOC cohort.

TABLE 16: subject status for a representative subset of subjects past day ⁺90. Abbreviations used: DLBCL, diffuse large B cell lymphoma; AML, acute myeloid leukemia; ALL, acute myeloid leukemia; MDS, myelodysplastic syndrome; MF, myelofibrosis; MPAL, mixed phenotype acute leukemia; CML, chronic myeloid leukemia; CR: complete remission; CR1, 1st complete remission; CR2, 2nd complete remission; active, active disease (not in CR as defined for given disease entity).

TABLE 16

| | Disease status | |
|---|---|---|
| Disease | at transplant | Most recent follow up and status |
| DLBCL | Active | Relapsed, day ⁺113 |
| AML | CR2 | Relapsed, day ⁺367 |
| ALL | Active | Alive, in remission, day ⁺1107 |
| ALL | CR2 | Alive, in remission, day ⁺1079 |
| CML | Active | Relapsed, day ⁺76, then responded |
| | | to donor lymphocyte infusion; |
| | | Alive, in remission, day ⁺1058 |
| AML | CR2 | Alive, in remission, day ⁺981 |
| MF | Active | Alive, in remission, day ⁺932 |
| AML | CR1 | Alive, in remission, day ⁺260 |
| PTCL | Active | Alive, in remission, day ⁺225 |
| AML | CR1 | Alive, in remission, day ⁺211 |
| AML | CR1 | Alive, in remission, day ⁺204 |
| MDS | Active | Alive, in remission, day ⁺190 |
| ALL | CR1 | Relapsed, day ⁺119 |
| ALL | CR2 | Alive, in remission, day ⁺127 |
| MF | Active | Alive, in remission, day ⁺119 |
| MPAL | CR1 | Alive, in remission, day ⁺120 |
| AML | CR1 | Alive, in remission, day ⁺99 |

The risk of relapse can be associated with disease status at the time of transplant. For example, the prognosis of AML or ALL subjects can be significantly worse if the subjects are not in complete remission at time of transplant (i.e., prognosis is worse if the subjects have active leukemia or detectable minimal residual disease at the time of transplant). In another example, prognosis is worse in subjects that have detectable minimal residual disease versus patients who do not have detectable minimal residual disease. Subjects with active disease or with minimal residual disease can represent a critical unmet medical need.

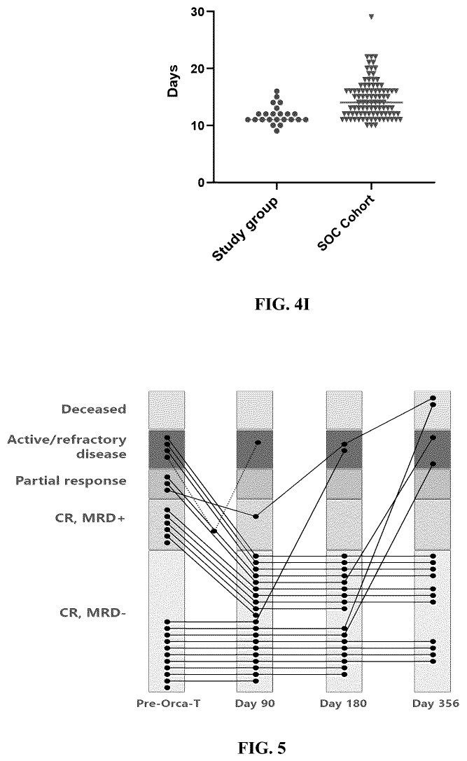

FIG. 5 summarizes the disease status of a subset of subjects in the study group before transplant and at day ⁺90, ⁺180, and ⁺356 post-transplant. Of the 10 subjects that received the composition of the disclosure, 7 achieved durable remissions, demonstrating a graft-versus tumor effect. Two of the subjects that relapsed had active disease at the time of transplant, and one had detectable minimal residual disease (MRD) at the time of transplant.

### J. Characteristics of Transplanted Cells

A schematic of graft production and administration for the sorting protocol is provided in FIG. 1A. Cell product from apheresis collection Day 2 was given an assessment and a portion of the apheresis product comprising a heterogenous cell component (e.g., a third population of CD45⁺ cells) comprising 3×10⁶ Tcon cells was administered to the subjects. A portion of the heterogenous cell component was analyzed for different cell components after staining for various cell populations as described in Table 17.

TABLE 17

| | Immunophenotype ((⁺) positive |
|---|---|
| Cell population | staining, (−) negative staining) |
| Granulocytes | CD45⁺, high side scatter, CD14− |
| Monocytes | CD45⁺, moderate side scatter, high |
| | forward scatter, CD14⁺ |
| Lymphocytes | CD45⁺, low forward and side scatter |
| PBMC | Defined as the total population of |
| | lymphocytes and monocytes |
| Lymphocyte subsets | |
| T cells | CD3⁺ lymphocytes |
| CD4⁺ T cells | CD4⁺, CD8− T cells |
| CD8⁺ T cells | CD8⁺, CD4− T cells |
| Naive T cells | CD45RA⁺CD45RO− T cells |
| | (can be subdivided based on CD4 and |
| | CD8 expression) |
| Memory T cells | CD45RA−CD45RO⁺ T cells |
| | (can be subdivided based on CD4 and |
| | CD8 expression) |
| Tregs | CD25⁺, CD127dimCD4⁺ T cells |
| NKT cells | CD56⁺ T cells |
| B cells | CD19⁺, CD3− lymphocytes |
| NK cells | CD56⁺, CD3− lymphocytes |
| CD34⁺ cells | CD45dim, CD34⁺, CD3− cells |
| (HSPCs) | that fall within the general lymphocyte |
| | scatter region |
| PBMC subsets | |
| Plasmacytoid dendritic | CD3−, CD19−, CD56−, CD14− (Lin−) |
| cells (pDCs) | cells that are CD4⁺, CD11b−, and BDCA2+ |
| Myeloid dendritic cells | CD3−, CD19−, CD56−, CD14− (Lin−) |
| (mDCs) | cells that are CD4⁺, CD11b⁺, and variably |
| | express CD16 |

Table 18 below illustrates data collected from a few patients from the study and identifies the various cell populations transplanted into a subject as part of the heterogenous cell component comprising 3×10⁶ Tcon cells.

TABLE 18
Heterogenous cell component

| | | | Standard | | | | | |
|---|---|---|---|---|---|---|---|---|
| Cell subset | Parent gate | Mean | Deviation | Median | Range | Minimum | Maximum | Count |
| Tcons | 45 Lymph | 60.05 | 8.67 | 58.6 | 33.4 | 47.6 | 81 | 22 |
| CD4⁺ T cells | T cells | 48.59 | 10.07 | 46.55 | 36.1 | 32.7 | 68.8 | 22 |
| CD8⁺ T cells | T cells | 42.45 | 10.27 | 42.1 | 32.7 | 24.7 | 57.4 | 22 |
| B-cells | Lymph | 18.62 | 6.37 | 19.25 | 25.09 | 5.71 | 30.8 | 22 |
| NK cells | 45⁺Lymph | 11.81 | 5.5 | 11.4 | 23.5 | 4.2 | 27.7 | 22 |
| NKT cells | T cells | 10.02 | 5.38 | 9.01 | 20.98 | 3.32 | 24.3 | 22 |
| Treg cells | T4 | 6.01 | 2.1 | 5.84 | 7.49 | 2.71 | 10.2 | 21 |
| Naïve T cells | T cells | 50.25 | 8.57 | 49.5 | 32.8 | 34.8 | 67.6 | 21 |
| DRp38pT4 | T4 | 3.81 | 2.64 | 2.99 | 10.81 | 1.09 | 11.9 | 22 |
| DRp38pT8 | T8 | 7.39 | 4.67 | 5.72 | 18.24 | 1.96 | 20.2 | 22 |
| CD34⁺ | 45⁺Lymph | 1.62 | 0.64 | 1.66 | 2.33 | 0.31 | 2.64 | 22 |
| HSPCs | | | | | | | | |
| Granulocytes | 45⁺ | 58.48 | 9.12 | 58.7 | 34.8 | 38.3 | 73.1 | 20 |
| and | | | | | | | | |
| Monocytes | | | | | | | | |
| Lymphocytes | 45⁺ | 28.64 | 10.02 | 28.75 | 34.4 | 10.4 | 44.8 | 20 |
| CD14⁺ | Total | 26.28 | 6.59 | 26.5 | 25 | 16.4 | 41.4 | 20 |
| pDC | Lin− | 6.66 | 5.05 | 4.43 | 17.53 | 1.97 | 19.5 | 12 |
| mDC | Lin− | 14.15 | 9.2 | 10.95 | 27.7 | 3.3 | 31 | 12 |

### Example 2: Analysis of Patients Receiving Different Conditioning Regimens

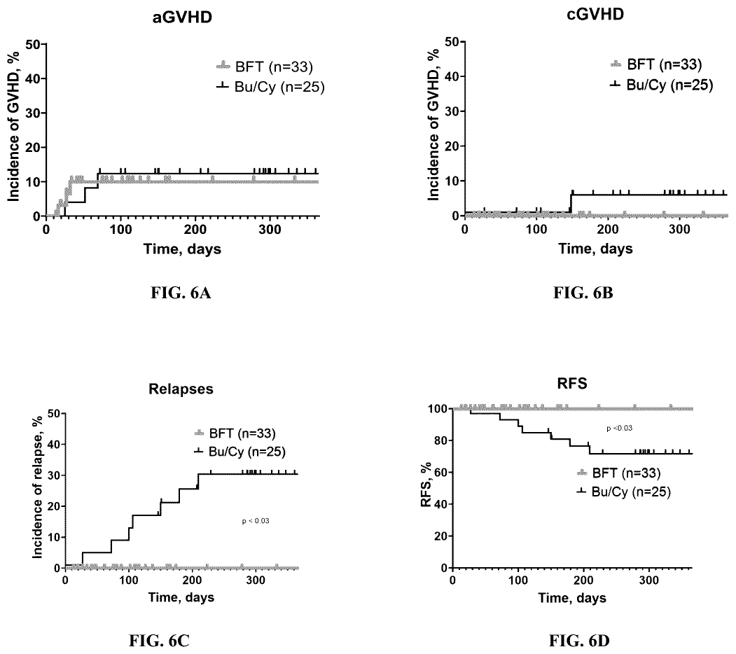

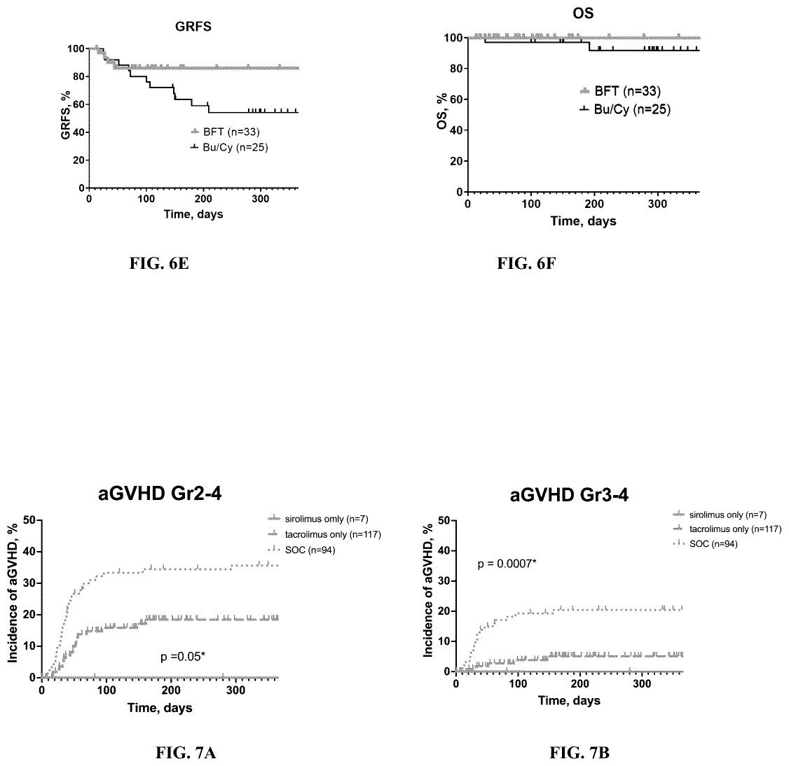

The data was further quantified for clinical outcomes in subjects that received different conditioning regimens. FIG. 6A compares acute GVHD in patients that received a Bu/Cy conditioning (see Table 14) vs a conditioning regimen comprising Thiotepa (BFT—busulfan, fludarabine and thiotepa; regimen described in Table 14). The acute GVHD rates were similar for the two regimens but were lower in patients that received thiotepa. A higher difference was seen in patients that received thiotepa in chronic GVHD (FIG. 6B). Surprisingly, the relapse rate in patients dropped significantly (p<0.03) in patients with the BFT regimen where no patients who received the BFT regimen relapsed (see FIGS. 6C and 6D). The GVHD and Relapse Free survival rates (GRFS) were also significantly lower in the BFT regimen patients (see FIG. 6E). The overall survival was also improved in the BFT-receiving patients (see FIG. 6F). These data demonstrate that conditioning regimens comprising thiotepa provide a clear and surprising advantage.

### Example 3: Analysis of Patients Receiving Different GVHD Prophylactic Agent Regimens

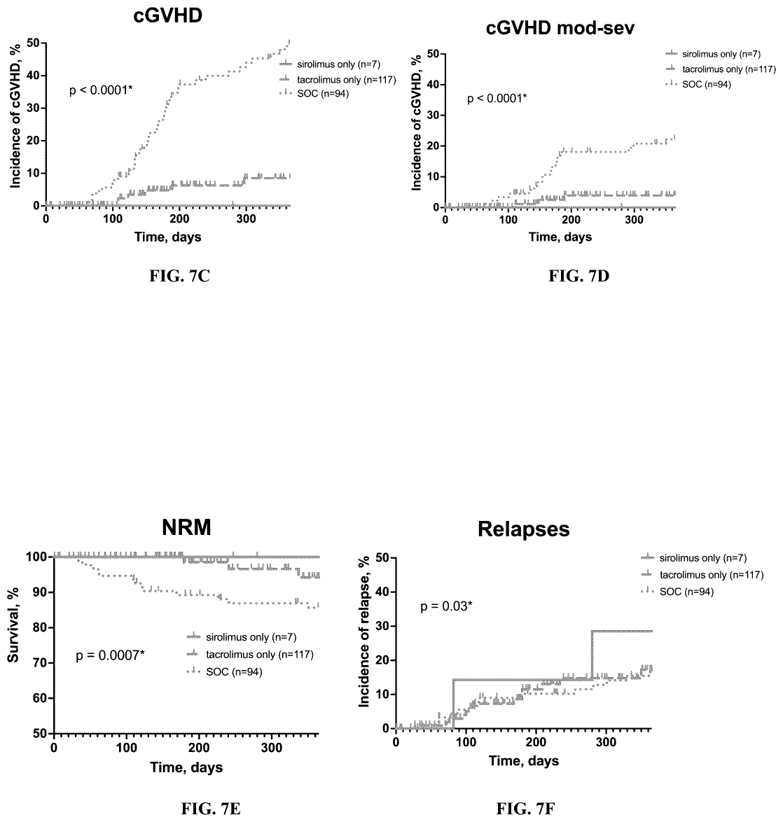

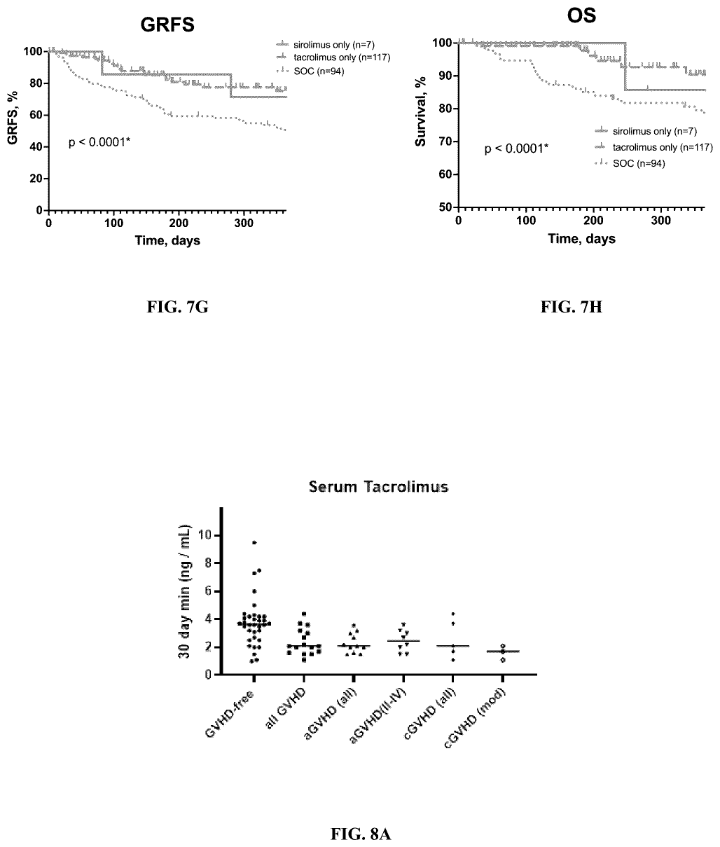

The data from the clinical trial was further quantified for clinical outcomes in subjects that received different GVHD prophylactic agents-sirolimus or tacrolimus. FIG. 7A compares acute GVHD in patients that received sirolimus only or tacrolimus only versus standard of care (SOC) patients that received a combination of methotrexate and tacrolimus (see FIG. 1B for SOC regimen). The patients that received only tacrolimus showed higher GVHD rates (FIGS. 7A-7C) than patients who received sirolimus. However, the survival (FIG. 7E), relapse rates (FIG. 7F), GRFS rates (FIG. 7G) and overall survival rates (FIG. 7H) were improved in tacrolimus-only patients relative to the sirolimus-only patients. As FIG. 7A shows, multiple drugs can be used as GHVD prophylaxis with significantly lower rates of acute GVHD versus standard of care.

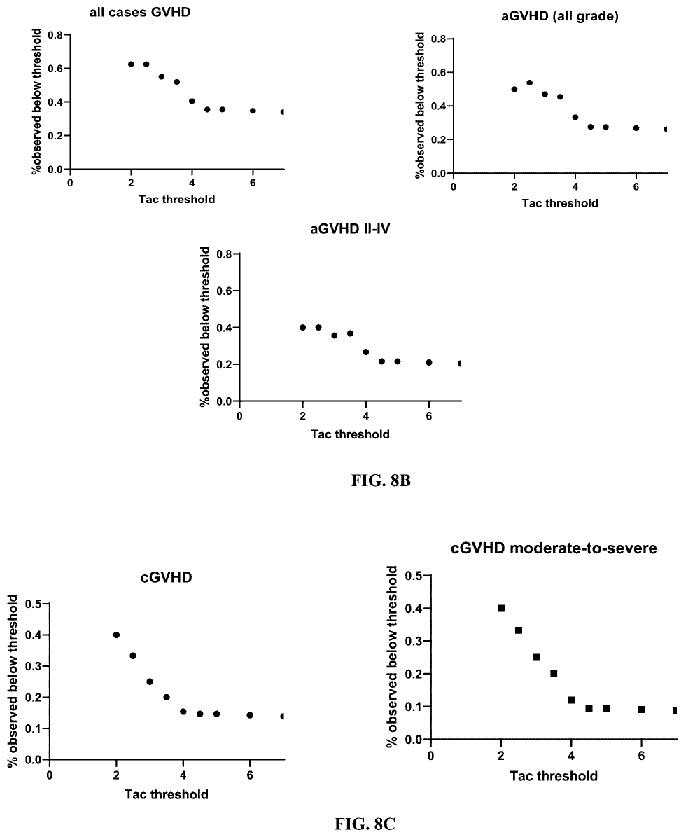

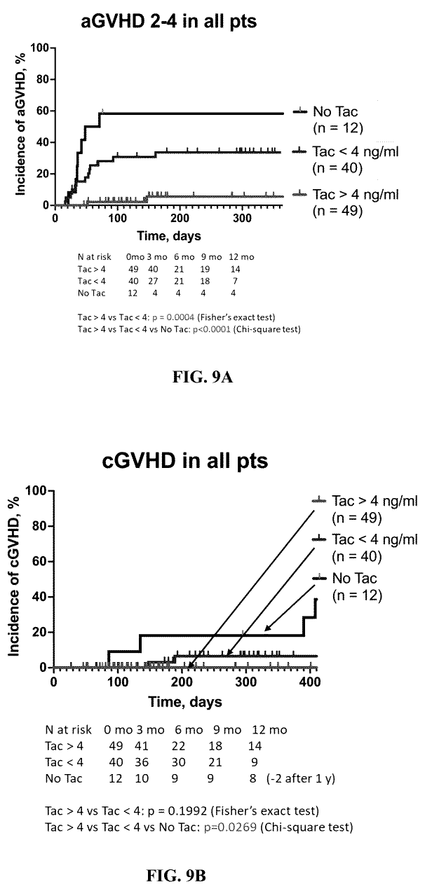

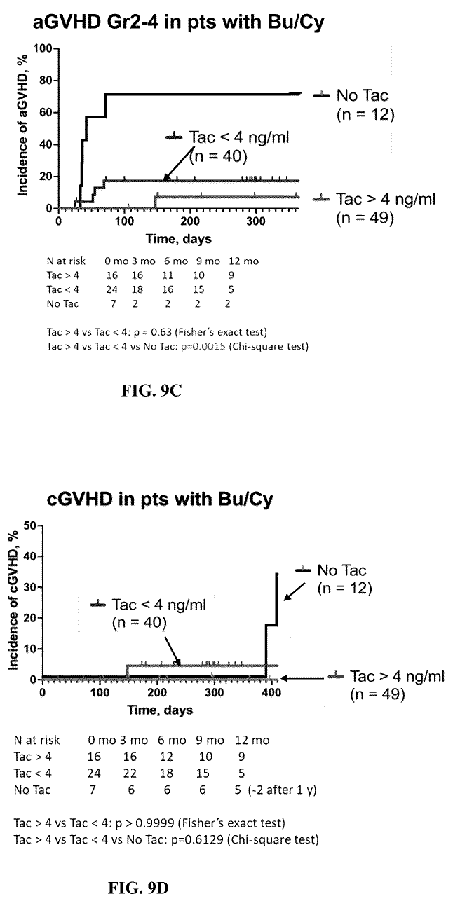

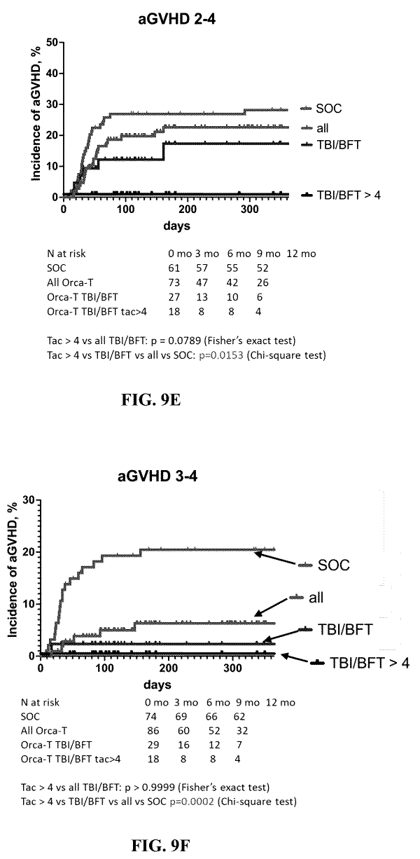

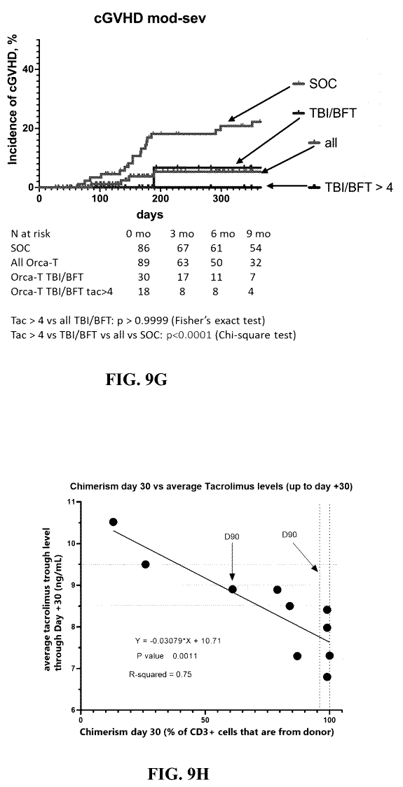

Upon further analysis, it was observed that patients had different serum trough levels. Serum tacrolimus levels had direct effects on the clinical outcome in the patient populations as is shown in FIGS. 8A-9G). As illustrated in FIG. 8A, patients who maintained an average serum tacrolimus trough level higher than 4 ng/ml for the first 30 days post-transplant had lower GVHD rates. FIGS. 8B-8C are derived from the data in FIG. 8A and further present data for acute GVHD and chronic GVHD rates. They illustrate a calculation of the probability of developing GVHD (y axis) if tacrolimus levels fall below the threshold value (x axis).

FIGS. 9A-9G illustrate direct comparison of clinical outcomes in patients that had a serum tacrolimus trough level higher or lower than 4 ng/ml. Each figure legend describes the number of patients and the time periods where their serum tacrolimus trough levels were higher or lower than 4 ng/ml. Acute GVHD rates showed significant improvement with higher tacrolimus levels (FIG. 9A) and chronic GVHD rates also showed improvements (FIG. 9B).

Patients who received the same conditioning regimen (Bu/Cy for FIGS. 9C-D; TBI/BFT for FIGS. 9E-G) and but had different serum tacrolimus trough levels (higher or lower than 4 ng/ml) were also analyzed for GVHD rates and results are illustrated in FIGS. 9C-G. In all instances, a higher tacrolimus trough level showed improvement in GVHD rates.

When T cell chimerism was compared to the average trough tacrolimus level through day ⁺30 post-transplant, a significant correlation was found; lower trough tacrolimus levels were associated with increased engraftment of donor T cells (p=0.0011). FIG. 9H shows the average trough tacrolimus level through day ⁺30 post-transplant for a small subset of patients, plotted against the proportion of CD3⁺ cells of donor origin at day ⁺30 (except that chimerism data is from day 90 where indicated by “D90”). These data suggest that maintaining lower circulating levels of tacrolimus can contribute to improved engraftment of donor T cells and improved donor T cell chimerism, which may contribute to improved relapse-free survival in allogeneic hematopoietic stem cell transplant recipients. Without wishing to be bound by a theory, it can be hypothesized that an improved chimerism due to higher tacrolimus levels may have led to the improved GVHD and survival outcomes in patients.

### Example 4: Overall Survival, GVHD Reduction, and Tolerability in Patients with Hematologic Malignancies

Graft versus host disease (GVHD) remains a leading cause of morbidity and mortality following allogeneic hematopoietic stem cell transplant (alloHSCT). Strategies to reduce GVHD have been limited in efficacy or compromised by increased risk of infection and relapse.

Orca-T is a high-precision, allogeneic investigational cell therapy product comprised of stem and immune cells that leverages highly purified, polyclonal donor regulatory T cells to control alloreactive immune responses. Details of the cell composition and methods are described in Examples 1-3. Orca-T shows a favorable safety profile and promising control of both GVHD and relapse.

Through 1 Mar. 2022, 137 patients with high-risk hematologic malignancies received Orca-T in a single-center Phase 2 study (NCT01660607, n=34) and multicenter Phase 1b study (NCT04013685, n=103) and have ≥100 days of follow-up (or died prior to Day ⁺100; range 27-1988 days). Patients underwent transplant between from July 2016 and December 2021. Median follow-up for patients is 341 days. Patients received investigator's choice of myeloablative conditioning regimens (busulfan-based, n=99; TBI-based, n=38) followed by single-agent prophylaxis with either tacrolimus (n=130) or sirolimus (n=7). For comparison purposes, an independent CIBMTR-based control arm was identified which included patients with hematologic malignancies who received myeloablative alloHSCT with a PBSC source over a similar timeframe (2016-2018) followed by tacrolimus plus methotrexate GVHD prophylaxis (n=375, 69 transplant centers across the United States). Patient demographics are shown in Table 19.

TABLE 19

| | | Orca-T | Orca-T | |
|---|---|---|---|---|
| | | (Multi- | (Single- | CIBMTR |
| | | center | Center | Control |
| | | Phase 1b) | Phase 1/2) | Cohort |
| Cohort size | | 103 | 34 | 375 |
| Median age (range) | | 51 (19-65) | 42 (19-71) | 52 (18-65) |
| % Male | | 52% | 71% | 57% |
| Race | White | 74% | 60% | 73% |
| African American | | 1% | 0% | 9% |
| Asian | | 11% | 20% | 12% |
| Unspecified/Unreported | | 14% | 20% | 6% |
| Primary | AML | 43% | 41% | 47% |

| | | | |
|---|---|---|---|
| Disease % | | | |
| ALL | 32% | 17% | 20% |
| MDS | 13% | 2% | 33% |
| myelofibrosis | 7% | 7% | 0% |
| CML | 3% | 12% | 0% |
| Non-Hodgkin Lymphoma | 0% | 10% | 0% |
| Other | 2% | 11% | 0% |
| (e g mixed phenotype acute | | | |
| leukemia) | | | |
| Myeloblative regimen: Busulfan- | 78%/22% | 76%/24% | 77%/20% |
| based/TBI-based | | | |
| Graft source: | 54%/46% | 74%/26% | 45%/55% |
| HLA-matched siblings/ | | | |
| HLA-matched unrelated donor | | | |
| Median f/u in days (range) | 313 days | 367 days | 900 days |
| | (27-859) | (104-1988) | (120-1500) |

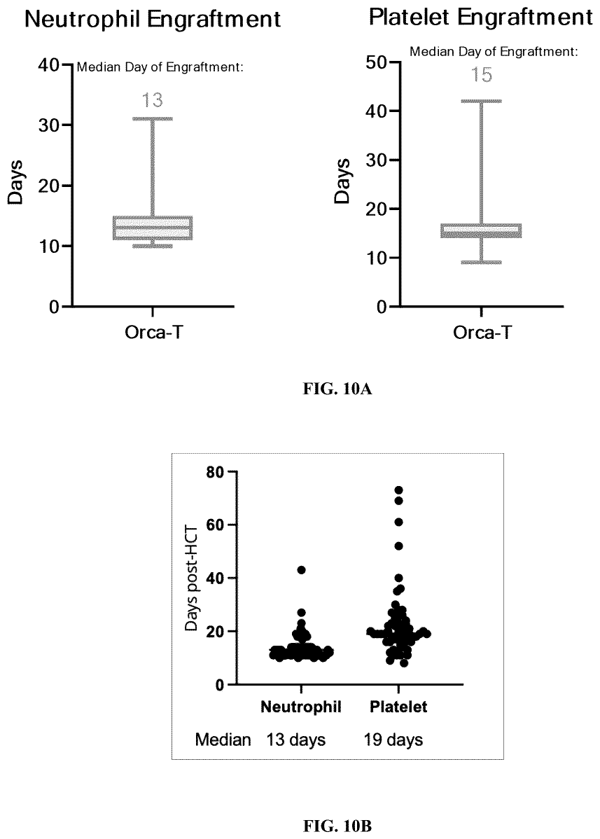

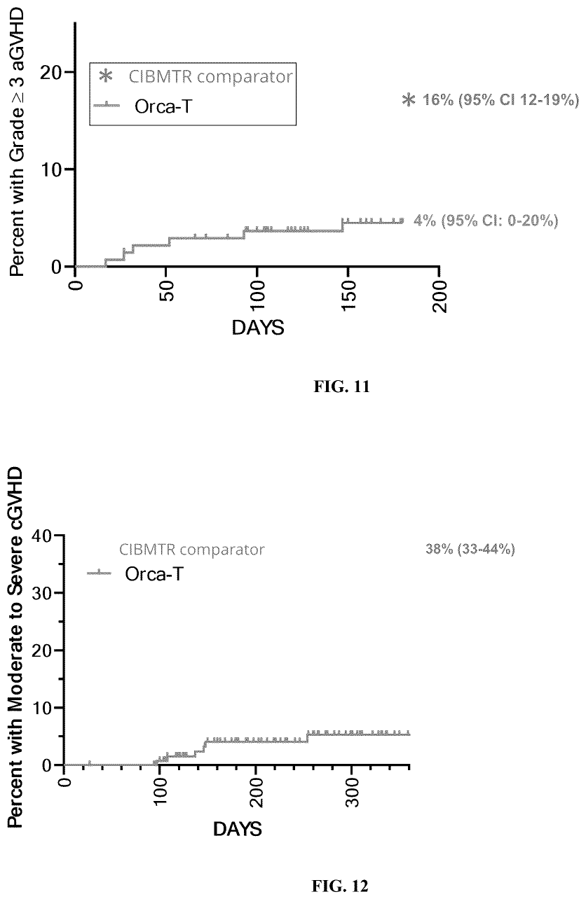

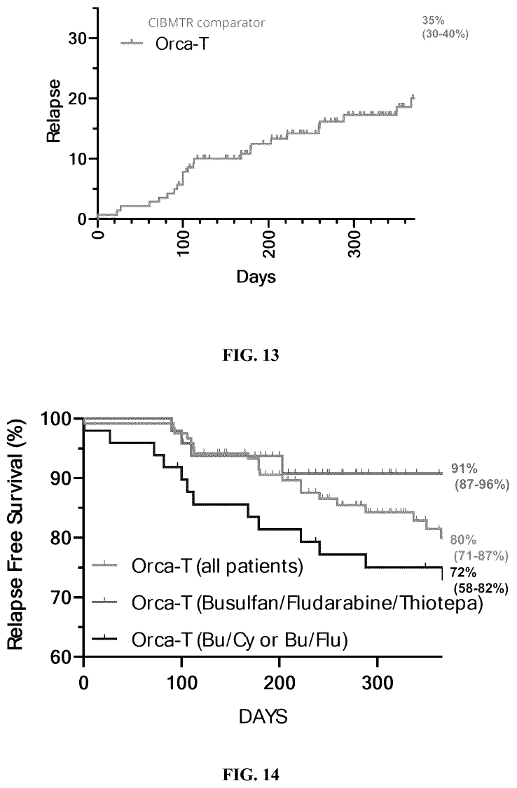

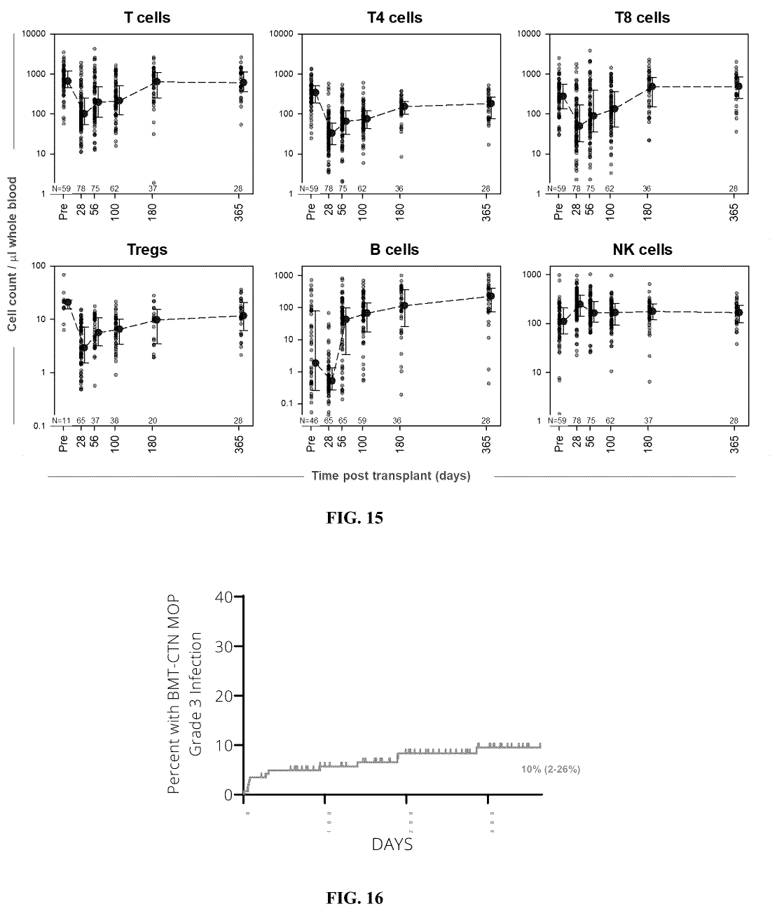

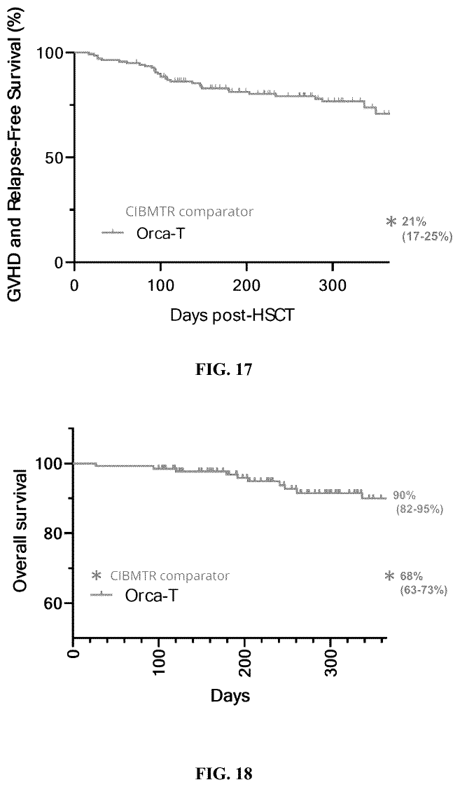

FIGS. 10-18 describe results of the clinical trial. Orca-T is a high-precision, allogeneic investigational cell therapy that replaces conventional alloHSCT. To date, an Orca-T product has been successfully produced for more than 160 patients. Compared to historic controls, Orca-T plus single-agent GVHD prophylaxis has led to reduced aGVHD and cGVHD without a concomitant increase in relapse or severe infection. Preliminary data suggests that immune reconstitution after Orca-T is robust. Experience with Orca-T in two Phase 1/2 trials suggests that this approach may yield significant improvements in GRFS and overall survival.

### Example 5: Orca-T Administration for the Treatment of Hematologic Malignancies in Patients with 7/8 HLA Mismatch Donors

Orca-T is a high-precision, allogeneic investigational cell therapy product comprised of stem and immune cells that leverages highly purified, polyclonal donor regulatory T cells to control alloreactive immune responses. Details of the cell composition and methods are described in Examples 1-3.

This is a single center Phase 2 clinical trial. The trial is described in Table 20 below.

TABLE 20

Single Center Phase 2 Trial (NCT01660607)
Acute leukemia (AML, ALL, mixed phenotype), in CR
Acute leukemia (AML, ALL, mixed phenotype),
with active disease at time of transplant
(≤10% BM blast burden)
Myelodysplastic syndrome
Myelofibrosis
BPDCN
CML in accelerated phase or blast crisis
Non-Hodgkin Lymphoma
8/8 or 7/8 matched related Qr unrelated donor (HLA-ARC DRB1)
HCT-CI ≤ 4
KPS ≥ 70
Age 18-72
Adequate organ function

To date, eight (8) patients have been treated with 7/8 HLA mismatch donors. Patient demographics are shown in Table 21.

TABLE 21

| | | | | | | | | | | Co- |
|---|---|---|---|---|---|---|---|---|---|---|
| Race/ | | | | | MRD status | R/D | ABO | Conditioning | HLA | morbidity |
| Ethnicity | Age | Sex | Disease | Disease Status | (pre) | CMV | compatibility | Regimen | MISMATCH | Score |
| White/Non- | 47 | M | MF | SD | n/a | −/+ | Minor | BU/FLU/TT | HLA-A. DPB1 | 1 |
| Hispanic | | | | | | | | | | |
| Asian/Non- | 28 | F | AML | CR | MRD+ | +/+ | Minor | BU/FLU/TT | HLA-A | 4 |
| Hispanic | | | | | | | | | | |
| Asian/Non- | 48 | M | CML | Complete | MRD− | +/− | Compatible | BU/FLU/TT | HLA-C. | 3 |
| Hispanic | | | | hematologic | | | | | DPB1 (perm) | |
| | | | | response (CHR) | | | | | | |
| | | | | preceded only by | | | | | | |
| | | | | chronic phase | | | | | | |
| Asian/Non- | 29 | F | AML | CR | MRD− | +/+ | Minor | BU/FLU/TT | HLA-A | 0 |
| Hispanic | | | | | | | | | | |
| Other/Hispanic | 29 | M | ALL | CR | MRD+ | +/+ | Minor | FTB/ | HLA-A | 0 |
| | | | | | | | | VP16CY | | |
| Other/Hispanic | 26 | F | ALL | CR | MRD+ | +/− | Compatible | FTBI/VP16/ | HLA-C. | 0 |
| | | | | | | | | CY | DPB1 (non- | |
| | | | | | | | | | perm) | |
| Other/Non- | 64 | M | MF | SD | n/a | +/+ | Minor | BU/FLU/TT | HLA-B - | 4 |
| Hispanic | | | | | | | | | permissible | |
| | | | | | | | | | DFB1 (perm) | |
| Asian/Non- | 59 | F | MDS, TP53 | NR/SD | MRD+ | +/+ | Bidirectional | BU/FLU/TT | HLA-C. | 3 |
| Hispanic | | | | | | | | | DPB1 (perm) | |

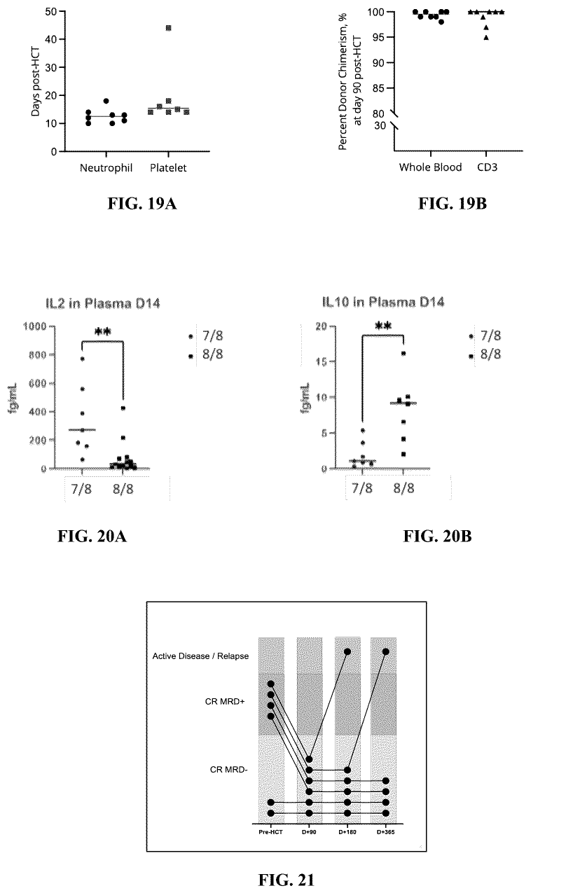

FIGS. 19-21 describe results of the clinical trial. FIGS. 19A and 19B show the time to neutrophil and platelet engraftment and the percent donor chimerism of whole blood and CD3⁺ T-cells. The median time to neutrophil engraftment was 12.5 days and the median time to platelet engraftment was 15.5 days post-engraftment (FIG. 19A). By day 90 post-transplant, there was full whole blood chimerism and over 90% CD3⁺ T-cell chimerism (FIG. 19B). FIG. 20A shows that the IL-2 serum plasma levels 14 days post-transplant were higher in patients with transplants from 7/8 HLA mismatched donors (median level was approximately 300 fg/ml) than those with transplants from 8/8 matched donors (median level was less than 100 fg·ml). FIG. 20B shows that the IL-10 serum plasma levels 14 days post-transplant were lower in patients with transplants from 7/8 HLA mismatched donors (median level was less than 3 fg/ml) than those with transplants from 8/8 matched donors (median level was approximately 10 fg/ml). FIG. 21 shows the clinical outcomes of patients that received Orca-T derived from donors that are 7/8 HLA mismatched at 90 days, 180 days, and 365 days post-transplant. The results are also summarized in Table 22.

TABLE 22

| Clinical Outcome | |
|---|---|
| (n = 8) | Events |
| Grade 1-2 Acute | 1 |
| GvHD | |
| Grade 3-4 Acute | 0 |
| GvHD | |
| Severe Chronic | 0 |
| GvHD | |
| Grade 3+ infection | 1 |
| Relapse | 2 |
| Non relapse | 0 |
| mortality | |
| Overall survival | 100% |
| Median follow-up | 426 days |
| time (range) | (300-654) |

These results show that to date there has been no non-relapse mortality (NRM) and 100% overall survival. These results are consistent with the very low NRM and high overall survival seen with Orca-T in patients with fully matched donors.

### Example 6: Updated Results on Overall Survival, GVHD Reduction, and Tolerability in Patients with Hematologic Malignancies

Orca-T is a high-precision, allogeneic investigational cell therapy product comprised of stem and immune cells that leverages highly purified, polyclonal donor regulatory T cells to control alloreactive immune responses. Details of the cell composition and methods are described in Examples 1-3.

Through 25 Oct. 2022, 151 patients with high-risk hematologic malignancies received Orca-T in a single-center Phase 2 study (NCT01660607, n=34) and multicenter Phase 1b study (NCT04013685, n=117) and have ≥100 days of follow-up. Median follow-up for patients is 15 months. Patients received investigator's choice of myeloablative conditioning regimens followed by single-agent prophylaxis with tacrolimus. For comparison purposes, an independent CIBMTR-based control arm was identified which included patients with hematologic malignancies who received myeloablative alloHSCT with a PBSC source over a similar timeframe followed by tacrolimus plus methotrexate GVHD prophylaxis (n=375, 69 transplant centers across the United States). Patient demographics are shown in Table 23.

TABLE 23

| | CIBMTR | | Phase 1b 2 | |
|---|---|---|---|---|
| | Control | | Orca-T | |
| | (n = 375) | | (n = 151) | |
| Median age (range), years | 52 | (18-65) | 48 | (19-71) |
| Median follow-up in months (range) | 30 | (4-50) | 15 | (3-43) |

| Primary Disease | AML | 47% | 44% |
|---|---|---|---|
| | ALL | 20% | 31% |
| | MDS | 3% | 15% |
| | Other | n/a | 9% |
| Male | | 57% | 57% |
| Donor (HLA | Related | 45% | 52% |
| matched) | Unrelated | 55% | 48% |
| Cond. Regimen | Busulfan-Based | 77% | 77% |
| | TBI-based | 20% | 23% |

Table 24 indicates that Orca-T population outperforms standard of care AlloHSCT. Outcomes with Orca-T appeared to be enhanced further with a busulfan, fludarabine, and thiotepa (BFT) conditioning regimen.

TABLE 24

| | |
|---|---|
| CIBMTR | Phase 1b/2 |
| Control | Orca-T |
| (n = 375) | (n = 151) |

| | 1 | 18 | 1 | 18 |
|---|---|---|---|---|
| | year | months | year | months |
| GVHD and Relapse-Free | 21% | 19% | 70% | 65% |
| Survival | | | | |
| Non-relapse mortality | 10% | 10% | 4% | 4% |
| Overall survival | 68% | 64% | 88% | 84% |

An additional 71 patients received Orca-T with a BFT conditioning regimen. Patient demographics are shown in Table 25.

TABLE 25

| | Orca -T | Orca-T+ | |
|---|---|---|---|
| | (Total | Bu/Flu/Thiotepa | |
| | n = 151) | (n = 71) | |
| Median age (range), years | 48 (19-71) | 53 | (19 71) |
| Orca-T (Total n= | | | |
| Median follow-up in months | 15 (13-43) | 14 | (4 35) |

| | | |
|---|---|---|
| (range) | | |
| Male, % | 57% | 55% |
| Donor (HLA-matched) (%) | | |
| Related | 52% | 51% |
| Unrelated | 40% | 49% |
| Conditioning regimen (%) | | |
| Busulfan-based | 77% | 100% |
| TBI based | 23% | 0% |
| DISEASE: | | |
| AML | | |
| ALL | 44% | 59% |
| Mixed phenotype acute | 31% | 11% |
| leukemia | 3% | 1% |
| MDS | 15% | 23% |
| CML | 5% | 6% |
| NHL | 1% | 0% |
| MRD STATUS (LEUKEMIA ONLY) | | |
| MRD+ | 19% | 27% |
| MRD− | 54% | 42% |
| Unknown MRD status | 27% | 31% |
| Active disease | 3% | 5% |

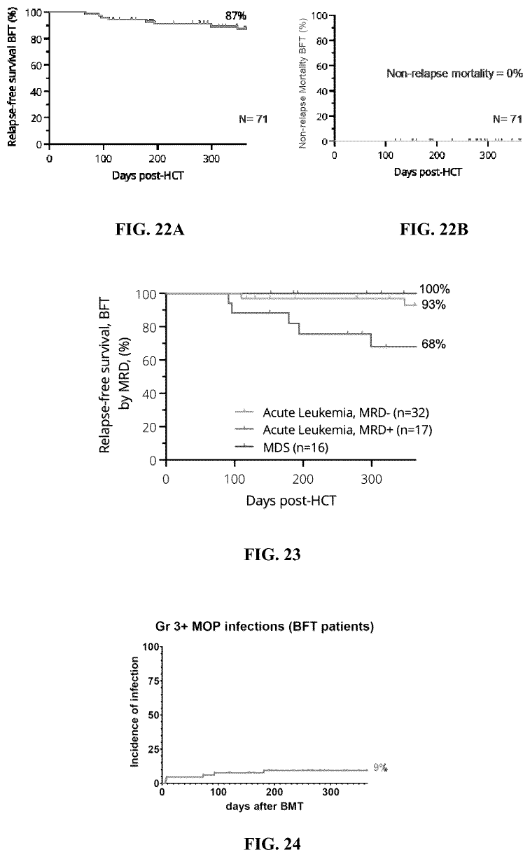

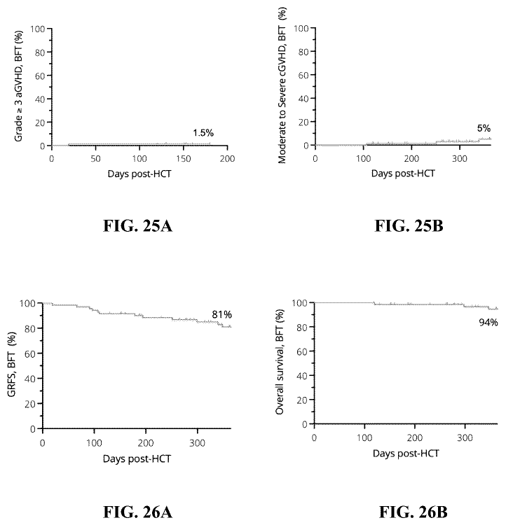

FIGS. 22-26 describe updated results from patients receiving a BFT conditioning regimen prior to treatment with Orca-T. FIG. 22A shows that 87% of patients exhibited relapse-free survival at 1 year, with a median follow-up time of 413 days. FIG. 22B shows that none of the patients experienced non-relapse mortality at 1 year post-transplant, with a median follow-up time of 413 days. FIG. 23 shows relapse-free survival at 1 year based on minimal residual disease (MRD) status, with a median follow-up time of 413 days. In particular, 16/16 patients with high-risk myelodysplastic syndrome (MDS) exhibited relapse-free survival, 83% of patients with MRD-negative acute leukemia with exhibited relapse-free survival, and 68% of patients with MRD-positive acute leukemia with exhibited relapse-free survival (FIG. 23). FIG. 24 shows that 9% of patients exhibited grade 3 or higher infections at 1 year post-transplant. FIG. 25A shows that 1.5% of patients exhibited grade 3 or higher aGVHD at over 150 days post-transplant. FIG. 25B shows that 5% of patients exhibited moderate-to-severe cGVHD at 1 year post-transplant. FIG. 26A shows that 81% of patients exhibited acute grade 3-4, chronic moderate-to-severe GVHD relapse-free survival (GFRS) at 1 year post-transplant. FIG. 26A shows that 94% of patients exhibited overall survival at 1 year post-transplant.

These results indicate that patients with acute leukemia and high risk MDS had robust graft-vs-leukemia and graft-vs-infection effects, very low incidence of GVHD, and markedly reduced treatment-related mortality (despite myeloablative conditioning) at over 1 year post-transplant with Orca-T.

### CONCLUSION

While various embodiments of the present disclosure have been shown and described herein, it will be obvious to those skilled in the art that such embodiments are provided by way of example only. Numerous variations, changes, and substitutions will be apparent to those skilled in the art without departing from the disclosure. For example, various embodiments of therapeutic compositions, methods of can be adapted for various pediatric, geriatric, and veterinary applications, the latter including one or more of felines, canines, and equines. Specific adaptions for such can include one or more of the cell types in the therapeutic composition administered to the patient and the dose of cells in the therapeutic composition. They can also be adapted for treatment of any number of hematologic and/or stem cell-based cancers including leukemia, lymphoma, myeloma, as well as a number of t-cell mediated and other autoimmune diseases including one more of multiple sclerosis, IBD, Celiac Disease, Crohn's disease, ulcerative colitis, ankylosing spondylosis, myasthenia gravis and diabetes. It should be understood that various alternatives to the embodiments of the present disclosure described herein may be employed in practicing the present disclosure.

Elements, characteristics, or acts from one embodiment can be readily recombined or substituted with one or more elements, characteristics or acts from other embodiments to form numerous additional embodiments within the scope of the present disclosure. Moreover, elements that are shown or described as being combined with other elements, characteristics, steps or acts, can, in various embodiments, exist as stand-alone elements, characteristics, steps or acts. Further, various embodiments expressly contemplate the negative recitation of any element, characteristic, step or act etc. that is/are shown or described in one or more embodiments. Hence, the scope of the present disclosure is not limited to the specifics of the described embodiments, but is instead limited solely by the appended claims.

1. A multi-component cellular therapy product comprising:
a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, wherein the first population of CD45⁺ cells is formulated with an excipient at a neutral pH;
b) a second single dose transfer bag comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the product, wherein the second population of isolated CD45⁺ cells is formulated with an excipient at a neutral pH, optionally wherein the first single dose transfer bag and the second single dose transfer bag are the same transfer bag; and
c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, wherein the third population of isolated CD45⁺ cells is formulated with an excipient at a neutral pH, wherein the excipient comprises one more cryoprotectants.

2. (canceled)

3. The multi-component cellular therapy product of claim 1, wherein the product further comprises a pharmaceutical composition comprising a dose of tacrolimus sufficient to maintain a trough blood level of approximately 5 ng/ml to approximately 10 ng/ml in the human subject receiving the product.

4. The multi-component cellular therapy product of claim 1, wherein:
the first population of isolated CD45⁺ cells, the second population of isolated CD45⁺ cells, and/or the third population of isolated CD45⁺ cells is formulated at a volume that ranges from approximately 5 mL to approximately 1 L.

5. The multi-component cellular therapy product of claim 1, wherein the first population of isolated CD45⁺ cells, the second population of isolated CD45⁺ cells, and the third population of isolated CD45+ cells are from an allogeneic donor having at least one HLA mismatch relative to the human subject receiving the product.

6. A method of treating a human subject having or suspected of having a hematologic malignancy, the method comprising administering to the human subject the multi-component cellular therapy product of claim 1.

7. A method of preparing a multi-component cellular therapy product comprising:
a) obtaining an HSPC-mobilized peripheral blood apheresis sample from a donor that is allogeneic with respect to a human subject receiving the product; and
b) selecting from the apheresis sample a first sample comprising from approximately 1.0×10⁵ to approximately 1.0×10⁸ CD34⁺ hematopoietic stem and progenitor cells (HSPCs) per kilogram of body weight of the human subject receiving the product, a second sample comprising from approximately 1.0×10⁵ to approximately 2.0×10⁷ CD4⁺CD25⁺CD127^{dim} regulatory T cells (Tregs) per kilogram of body weight of the human subject receiving the product, and a third sample comprising from approximately 1.0×10⁵ to approximately 4.0×10⁷ conventional CD3⁺ T cells (Tcons) per kilogram of body weight of the human subject receiving the product, wherein the multi-component cellular therapy product comprises the Tcons, the HSPCs, and the Tregs, wherein the HSPCs are selected by sorting the first sample with one or more ISPs specific for CD34 and selecting a CD34-enriched population, wherein the Tregs are selected by soring the second sample with one or more ISPs specific for CD25, one or more ISPs specific for CD4, and/or one or more ISPs specific for CD127, and selecting the CD4⁺ and CD127^{dim} cell population.

8. A multi-component cellular therapy product produced by the method of claim 7.

9. A method of treating a human subject having or suspected of having acute leukemia in complete remission, active leukemia, primary refractory acute leukemia, or acute leukemia with minimal residual disease, wherein the method comprises administering to the human subject the multi-component cellular therapy product of claim 1.

10. A method of treating a human subject having or suspected of having low-risk acute myeloid leukemia (AML), high-risk AML in complete remission, or chronic myelogenous leukemia (CML), wherein the method comprises administering to the human subject the multi-component cellular therapy product of claim 1.

11. A method of treating a human subject having or suspected of having high-risk myelodysplastic syndrome, therapy-related myelodysplastic syndrome, and/or secondary myelodysplastic syndrome, wherein the method comprises administering to the human subject the multi-component cellular therapy product of claim 1.

12. A method of treating a human subject having or suspected of having a myeloproliferative syndrome, the method comprising administering to the human subject the multi-component cellular therapy product of claim 1.

13. A method of treating a human subject having or suspected of having non-Hodgkin lymphoma with poor risk features not suitable for autologous hematopoietic cell transplant (HCT), the method comprising administering to the human subject the multi-component cellular therapy product of claim 1.

14. The method of claim 6, wherein the third population of isolated CD45⁺ cells is administered from approximately 24 to approximately 120 hours after administration of the first population of isolated CD45⁺ cells and the second population of isolated CD45⁺ cells.

15. A cellular therapy kit comprising the multi-component cellular therapy product of claim 1, wherein the kit further comprises written instructions for using the cellular therapy for treating a hematologic malignancy in a human subject.

16. The multi-component cellular therapy product of claim 1, wherein the neutral pH ranges from approximately 6.8 to approximately 7.6.

17. The multi-component cellular therapy product of claim 1, wherein the excipient comprises a transport buffer, wherein the transport buffer is selected from the group consisting of: phosphate-buffered saline (PBS), human serum, PlasmaLyte, and any combination thereof.

18. The multi-component cellular therapy product of claim 17, wherein the transport buffer further comprises approximately 0.1% weight by volume to approximately 10% weight by volume of a human carrier protein, wherein the human carrier protein is selected from the group consisting of: human serum albumin (HSA), intravenous immune globulin (IVIG), AB serum, and any combination thereof.

19. The multi-component cellular therapy product of claim 1, wherein any of the first single dose bag, the second single dose bad, and the third single dose bag is a polyvinyl chloride (PVC) transfer bag or an ethylene vinyl acetate (EVA) transfer bag.

20. The multi-component cellular therapy product of claim 1, wherein the one or more cryoprotectants are selected from the group consisting of: sorbitol, dimethyl sulfoxide (DMSO), propylene glycol, glycerol, polyvinylpyrrolidone (PVP), and polyethylene glycol (PEG), serum, HSA, hetastarch, CRYOSTOR CS2, CRYOSTOR CS5, and CRYOSTOR CS10.

21. The method of claim 6, wherein the administering comprises infusing into the human subject the first population of isolated CD45+ cells, the second population of isolated CD45+ cells, and the third population of isolated CD45+ cells.
